SEQUENCE LISTING (1165 sequences)

SEQ ID NO: 1 - protein, 972 aa, Artificial Sequence
  full length human KIT (GenBank accession number AAC50969) (see Fig. 1)
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 2 - protein, 11 aa, Artificial Sequence
  VL CDR1 of Ab1 and Ab61
QASQDIGNYLN

SEQ ID NO: 3 - protein, 11 aa, Artificial Sequence
  VL CDR1 of Ab2 (Table 1A)
RASQSVSNYVA

SEQ ID NO: 4 - protein, 13 aa, Artificial Sequence
  VL CDR1 of Ab3 (Table 1A)
SGSSSNIGNNYVY

SEQ ID NO: 5 - protein, 11 aa, Artificial Sequence
  VL CDR1 of Ab4, Ab38, Ab90, Ab121, Ab143, and Ab170
RASQSIDTYLN

SEQ ID NO: 6 - protein, 11 aa, Artificial Sequence
  VL CDR1 of Ab5 (Table 1A)
RASQSIDTYLN

SEQ ID NO: 7 - protein, 12 aa, Artificial Sequence
  VL CDR1 of Ab6 (Table 1A)
RASQSVGSSYLA

SEQ ID NO: 8 - protein, 11 aa, Artificial Sequence
  VL CDR1 of Ab7 (Table 1A)
RASQSIDTYLN

SEQ ID NO: 9 - protein, 11 aa, Artificial Sequence
  VL CDR1 of Ab8 (Table 1A)
RASQSVSFNLA

SEQ ID NO: 10 - protein, 11 aa, Artificial Sequence
  VL CDR1 of Ab9, Ab31, Ab41, Ab53, Ab57, Ab75, Ab80, Ab87, Ab88, Ab130, Ab134, Ab147, Ab179, Ab183 and Ab185
RASQSVSSYLA

SEQ ID NO: 11 - protein, 11 aa, Artificial Sequence
  VL CDR1 of Ab10 (Table 1A)
RASQSVRGNLA

SEQ ID NO: 12 - protein, 12 aa, Artificial Sequence
  VL CDR1 of Ab11, Ab45, Ab48, Ab60, Ab68, Ab69, Ab86, Ab94, Ab117, Ab145, Ab152, Ab171, and Ab180
RASQSVSSSYLA

SEQ ID NO: 13 - protein, 12 aa, Artificial Sequence
  VL CDR1 of Ab12 (Table 1A)
RASQSVKSNYLA

SEQ ID NO: 14 - protein, 7 aa, Artificial Sequence
  VL CDR2 of Ab1 and Ab61
DASFLKK

SEQ ID NO: 15 - protein, 7 aa, Artificial Sequence
  VL CDR2 of Ab2 (Table 1A)
DTSKRAT

SEQ ID NO: 16 - protein, 7 aa, Artificial Sequence
  VL CDR2 of Ab3 (Table 1A)
RSNQRPS

SEQ ID NO: 17 - protein, 7 aa, Artificial Sequence
  VL CDR2 of Ab4, Ab38, Ab54, Ab66, Ab90, Ab121, Ab143, Ab170 and Ab172
AASKLED

SEQ ID NO: 18 - protein, 7 aa, Artificial Sequence
  VL CDR2 of Ab5 (Table 1A)
AASKLED

SEQ ID NO: 19 - protein, 7 aa, Artificial Sequence
  VL CDR2 of Ab6 (Table 1A)
DASSRSS

SEQ ID NO: 20 - protein, 7 aa, Artificial Sequence
  VL CDR2 of Ab7 (Table 1A)
AASKLED

SEQ ID NO: 21 - protein, 7 aa, Artificial Sequence
  VL CDR2 of Ab8 (Table 1A)
DASTRAI

SEQ ID NO: 22 - protein, 7 aa, Artificial Sequence
  VL CDR2 of Ab9, Ab31, Ab41, Ab53, Ab57, Ab58, Ab75, Ab80, Ab87, Ab88, Ab119, Ab130, Ab134, Ab141, Ab147, Ab179, Ab183 and Ab185
DASNRAT

SEQ ID NO: 23 - protein, 7 aa, Artificial Sequence
  VL CDR2 of Ab10, Ab24, Ab32, Ab67, Ab77, Ab91, Ab146, and Ab182
GASTRAT

SEQ ID NO: 24 - protein, 7 aa, Artificial Sequence
  VL CDR2 of Ab11, Ab45, Ab68, Ab69, Ab83, Ab86, Ab100, Ab117, Ab120, Ab142, Ab145, Ab148, Ab152, Ab157 and Ab171
GASSRAT

SEQ ID NO: 25 - protein, 7 aa, Artificial Sequence
  VL CDR2 of Ab12 (Table 1A)
GASTRAT

SEQ ID NO: 26 - protein, 9 aa, Artificial Sequence
  VL CDR3 of Ab1 and Ab61
QHSDNLSVT

SEQ ID NO: 27 - protein, 8 aa, Artificial Sequence
  VL CDR3 of Ab2 (Table 1A)
QQRSYWFA

SEQ ID NO: 28 - protein, 11 aa, Artificial Sequence
  VL CDR3 of Ab3 (Table 1A)
AAWDDSLIGPV

SEQ ID NO: 29 - protein, 10 aa, Artificial Sequence
  VL CDR3 of Ab4, Ab38, Ab54, Ab66, Ab90, Ab121, Ab143, and Ab170
QQSYSSPGIT

SEQ ID NO: 30 - protein, 10 aa, Artificial Sequence
  VL CDR3 of Ab5 (Table 1A)
QQSYSSPGIT

SEQ ID NO: 31 - protein, 10 aa, Artificial Sequence
  VL CDR3 of Ab6 (Table 1A)
QQRSNRPPWT

SEQ ID NO: 32 - protein, 10 aa, Artificial Sequence
  VL CDR3 of Ab7 (Table 1A)
QQSYSSPGIT

SEQ ID NO: 33 - protein, 10 aa, Artificial Sequence
  VL CDR3 of Ab8 (Table 1A)
QQYNYPASYT

SEQ ID NO: 34 - protein, 9 aa, Artificial Sequence
  VL CDR3 of Ab9, Ab75, and Ab183
QQRSNGGIT

SEQ ID NO: 35 - protein, 10 aa, Artificial Sequence
  VL CDR3 of Ab10 (Table 1A)
QQYDSWPPFT

SEQ ID NO: 36 - protein, 10 aa, Artificial Sequence
  VL CDR3 of Ab11 (Table 1A)
QQYGSSRTWT

SEQ ID NO: 37 - protein, 10 aa, Artificial Sequence
  VL CDR3 of Ab12 (Table 1A)
QQYGSSLSYT

SEQ ID NO: 38 - protein, 24 aa, Artificial Sequence
  VL FR1 of Ab1 (Table 2A)
QDIQMTQSPTSLSAFVGDRVTITC

SEQ ID NO: 39 - protein, 24 aa, Artificial Sequence
  VL FR1 of Ab2 (Table 2A)
QDIQMTQSPATLSLSPGERATLYC

SEQ ID NO: 40 - protein, 22 aa, Artificial Sequence
  VL FR1 of Ab3 (Table 2A)
QSALTQPPSASGTPGQRVTISC

SEQ ID NO: 41 - protein, 24 aa, Artificial Sequence
  VL FR1 of Ab4 (Table 2A)
QDIQMTQSPSSLSASVGDRVAITC

SEQ ID NO: 42 - protein, 24 aa, Artificial Sequence
  VL FR1 of Ab5 (Table 2A)
QDIQMTQSPSSLSASVGDRVAITC

SEQ ID NO: 43 - protein, 24 aa, Artificial Sequence
  VL FR1 of Ab6 (Table 2A)
QDIQMTQSPATLSLYPGERATLSC

SEQ ID NO: 44 - protein, 24 aa, Artificial Sequence
  VL FR1 of Ab7 (Table 2A)
QDIQMTQSPSSLSASVGDRVAITC

SEQ ID NO: 45 - protein, 24 aa, Artificial Sequence
  VL FR1 of Ab8 (Table 2A)
QDIQMTQSPVTLSLSPGDRATLSC

SEQ ID NO: 46 - protein, 24 aa, Artificial Sequence
  VL FR1 of Ab9 (Table 2A)
QDIQMTQSPATLSLSPGERATLSC

SEQ ID NO: 47 - protein, 24 aa, Artificial Sequence
  VL FR1 of Ab10 (Table 2A)
QDIQMTQSPDSLSVSPGERATLSC

SEQ ID NO: 48 - protein, 24 aa, Artificial Sequence
  VL FR1 of Ab11 (Table 2A)
QDIQMTQSPGTLSLSPGERATLSC

SEQ ID NO: 49 - protein, 24 aa, Artificial Sequence
  VL FR1 of Ab12 (Table 2A)
QDIQMTQSPGTLSLSPGERATLYC

SEQ ID NO: 50 - protein, 15 aa, Artificial Sequence
  VL FR2 of Ab1 (Table 2A)
WYQQKSGEPPKLLVY

SEQ ID NO: 51 - protein, 15 aa, Artificial Sequence
  VL FR2 of Ab2 (Table 2A)
WYQQRPGQAPRLLMY

SEQ ID NO: 52 - protein, 15 aa, Artificial Sequence
  VL FR2 of Ab3 (Table 2A)
WYQQLPGTAPKLLIY

SEQ ID NO: 53 - protein, 15 aa, Artificial Sequence
  VL FR2 of Ab4 (Table 2A)
WYQQKPGKAPKLLIY

SEQ ID NO: 54 - protein, 15 aa, Artificial Sequence
  VL FR2 of Ab5 (Table 2A)
WYQQKPGKAPKLLIY

SEQ ID NO: 55 - protein, 15 aa, Artificial Sequence
  VL FR2 of Ab6 (Table 2A)
WYQQKPGQAPRLLIY

SEQ ID NO: 56 - protein, 15 aa, Artificial Sequence
  VL FR2 of Ab7 (Table 2A)
WYQQKPGKAPKLLIY

SEQ ID NO: 57 - protein, 15 aa, Artificial Sequence
  VL FR2 of Ab8 (Table 2A)
WYQHKPGQAPRLLMF

SEQ ID NO: 58 - protein, 15 aa, Artificial Sequence
  VL FR2 of Ab9 (Table 2A)
WYQQKPGQAPRLLIY

SEQ ID NO: 59 - protein, 15 aa, Artificial Sequence
  VL FR2 of Ab10 (Table 2A)
WYQQKPGQAPRLLIH

SEQ ID NO: 60 - protein, 15 aa, Artificial Sequence
  VL FR2 of Ab11 (Table 2A)
WYQQKPGQAPRLLIY

SEQ ID NO: 61 - protein, 15 aa, Artificial Sequence
  VL FR2 of Ab12 (Table 2A)
WYQQKPGQAPRLLIY

SEQ ID NO: 62 - protein, 32 aa, Artificial Sequence
  VL FR3 of Ab1 (Table 2A)
GVPSRFSGSGSGTQYFLTIYSLQPEDFATYFC

SEQ ID NO: 63 - protein, 32 aa, Artificial Sequence
  VL FR3 of Ab2 (Table 2A)
GIPARFSGSGSGTDFTLTISSLEPEDFAVYYC

SEQ ID NO: 64 - protein, 32 aa, Artificial Sequence
  VL FR3 of Ab3 (Table 2A)
GVPDRFSGSKSGASASLAISGLRSEDEADYYC

SEQ ID NO: 65 - protein, 32 aa, Artificial Sequence
  VL FR3 of Ab4 (Table 2A)
GVPSRFSGSGTGTDFTLTIRSLQPEDFASYFC

SEQ ID NO: 66 - protein, 32 aa, Artificial Sequence; identical to SEQ ID NO: 65
  VL FR3 of Ab5 (Table 2A)

SEQ ID NO: 67 - protein, 32 aa, Artificial Sequence
  VL FR3 of Ab6 (Table 2A)
GIPDRFSGSGSGTDFTLTITRLEPEDFAVYYC

SEQ ID NO: 68 - protein, 32 aa, Artificial Sequence; identical to SEQ ID NO: 65
  VL FR3 of Ab7 (Table 2A)

SEQ ID NO: 69 - protein, 32 aa, Artificial Sequence
  VL FR3 of Ab8 (Table 2A)
GTPDRFSGSGSGTEFTLTISSLQSEDSAVYYC

SEQ ID NO: 70 - protein, 32 aa, Artificial Sequence; identical to SEQ ID NO: 63
  VL FR3 of Ab9 (Table 2A)

SEQ ID NO: 71 - protein, 32 aa, Artificial Sequence
  VL FR3 of Ab10 (Table 2A)
DIPTRFSGSGSGTDFTLTISSMQSEDFAVYYC

SEQ ID NO: 72 - protein, 32 aa, Artificial Sequence
  VL FR3 of Ab11 (Table 2A)
GIPDRFSGSGSGTDFTLTISRLEPEDFAVYYC

SEQ ID NO: 73 - protein, 32 aa, Artificial Sequence
  VL FR3 of Ab12 (Table 2A)
DIPDRFSGSGSGTDFTLTISRLEPEDFAVYYC

SEQ ID NO: 74 - protein, 10 aa, Artificial Sequence
  VL FR4 of Ab1 (Table 2A)
FGGGTKVEVK

SEQ ID NO: 75 - protein, 10 aa, Artificial Sequence
  VL FR4 of Ab2 (Table 2A)
FGGGTKVEIK

SEQ ID NO: 76 - protein, 10 aa, Artificial Sequence
  VL FR4 of Ab3 (Table 2A)
FGGGTKLTVL

SEQ ID NO: 77 - protein, 10 aa, Artificial Sequence
  VL FR4 of Ab4 (Table 2A)
FGPGTKVEIK

SEQ ID NO: 78 - protein, 10 aa, Artificial Sequence
  VL FR4 of Ab5 (Table 2A)
FGPGTKVEIK

SEQ ID NO: 79 - protein, 10 aa, Artificial Sequence
  VL FR4 of Ab6 (Table 2A)
FGQGTKVEIK

SEQ ID NO: 80 - protein, 10 aa, Artificial Sequence
  VL FR4 of Ab7 (Table 2A)
FGPGTKVEIK

SEQ ID NO: 81 - protein, 10 aa, Artificial Sequence
  VL FR4 of Ab8 (Table 2A)
FGQGTKLEIS

SEQ ID NO: 82 - protein, 10 aa, Artificial Sequence
  VL FR4 of Ab9(Table 2A)
FGQGTRLEIK

SEQ ID NO: 83 - protein, 10 aa, Artificial Sequence
  VL FR4 of Ab10 (Table 2A)
FGPGTKVDIK

SEQ ID NO: 84 - protein, 10 aa, Artificial Sequence
  VL FR4 of Ab11 (Table 2A)
FGQGTKVEIK

SEQ ID NO: 85 - protein, 10 aa, Artificial Sequence
  VL FR4 of Ab12 (Table 2A)
FGQGTKLEIK

SEQ ID NO: 86 - protein, 5 aa, Artificial Sequence
  VH CDR1 of Ab1 and Ab61
NYLMS

SEQ ID NO: 87 - protein, 5 aa, Artificial Sequence
  VH CDR1 of Ab2 (Table 3A)
NYKMG

SEQ ID NO: 88 - protein, 5 aa, Artificial Sequence
  VH CDR1 of Ab3 (Table 3A)
PYNMM

SEQ ID NO: 89 - protein, 5 aa, Artificial Sequence
  VH CDR1 of Ab4 (Table 3A)
WYWMY

SEQ ID NO: 90 - protein, 5 aa, Artificial Sequence
  VH CDR1 of Ab5 (Table 3A)
SYPMA

SEQ ID NO: 91 - protein, 5 aa, Artificial Sequence
  VH CDR1 of Ab6 (Table 3A)
YYSMG

SEQ ID NO: 92 - protein, 5 aa, Artificial Sequence
  VH CDR1 of Ab7 (Table 3A)
RYPMN

SEQ ID NO: 93 - protein, 5 aa, Artificial Sequence
  VH CDR1 of Ab8 and Ab120
NYKMQ

SEQ ID NO: 94 - protein, 5 aa, Artificial Sequence
  VH CDR1 of Ab9 (Table 3A)
NYLMQ

SEQ ID NO: 95 - protein, 5 aa, Artificial Sequence
  VH CDR1 of Ab10 (Table 3A)
IYSMM

SEQ ID NO: 96 - protein, 5 aa, Artificial Sequence
  VH CDR1 of Ab11, Ab67 and Ab123
NYRMM

SEQ ID NO: 97 - protein, 5 aa, Artificial Sequence
  VH CDR1 of Ab12 (Table 3A)
FYKMH

SEQ ID NO: 98 - protein, 17 aa, Artificial Sequence
  VH CDR2 of Ab1 and Ab61
SIVPSGGFTHYADSVKG

SEQ ID NO: 99 - protein, 17 aa, Artificial Sequence
  VH CDR2 of Ab2 (Table 3A)
WIYPSGGGTFQADSVKG

SEQ ID NO: 100 - protein, 17 aa, Artificial Sequence
  VH CDR2 of Ab3 (Table 3A)
RISPSGGDTHYADSVKG

SEQ ID NO: 101 - protein, 17 aa, Artificial Sequence
  VH CDR2 of Ab4 (Table 3A)
GIYPSGGFTSYADSVKG

SEQ ID NO: 102 - protein, 17 aa, Artificial Sequence
  VH CDR2 of Ab5 (Table 3A)
VISSSGGYTPYADSVKG

SEQ ID NO: 103 - protein, 17 aa, Artificial Sequence
  VH CDR2 of Ab6 (Table 3A)
VIRPSGGHTHYADSVKG

SEQ ID NO: 104 - protein, 17 aa, Artificial Sequence
  VH CDR2 of Ab7 (Table 3A)
GIRSSGGATSYADSVKG

SEQ ID NO: 105 - protein, 17 aa, Artificial Sequence
  VH CDR2 of Ab8 (Table 3A)
YISSSGGYTSYADSVKG

SEQ ID NO: 106 - protein, 17 aa, Artificial Sequence
  VH CDR2 of Ab9 (Table 3A)
GIVPSGGSTVYADSVKG

SEQ ID NO: 107 - protein, 17 aa, Artificial Sequence
  VH CDR2 of Ab10 (Table 3A)
WIVSSGGQTYYADSVKG

SEQ ID NO: 108 - protein, 17 aa, Artificial Sequence
  VH CDR2 of Ab11, Ab67 and Ab123
GIYPSGGGTFYADSVKG

SEQ ID NO: 109 - protein, 17 aa, Artificial Sequence
  VH CDR2 of Ab12 (Table 3A)
GIWPSGGYTEYADSVKG

SEQ ID NO: 110 - protein, 13 aa, Artificial Sequence
  VH CDR3 of Ab1 (Table 3A)
LQTGSWRVHAFDI

SEQ ID NO: 111 - protein, 11 aa, Artificial Sequence
  VH CDR3 of Ab2 (Table 3A)
VRVGSTGNFDY

SEQ ID NO: 112 - protein, 11 aa, Artificial Sequence
  VH CDR3 of Ab3 (Table 3A)
GSRSSWSTPYY

SEQ ID NO: 113 - protein, 17 aa, Artificial Sequence
  VH CDR3 of Ab4 (Table 3A)
SYYYGSGSYYNSASFDY

SEQ ID NO: 114 - protein, 8 aa, Artificial Sequence
  VH CDR3 of Ab5 (Table 3A)
RYGGGMDV

SEQ ID NO: 115 - protein, 8 aa, Artificial Sequence
  VH CDR3 of Ab6 (Table 3A)
LATNAFDI

SEQ ID NO: 116 - protein, 8 aa, Artificial Sequence
  VH CDR3 of Ab7 (Table 3A)
SFGRAFDY

SEQ ID NO: 117 - protein, 18 aa, Artificial Sequence
  VH CDR3 of Ab8 (Table 3A)
VAHQYYDFWSGYYGAFDI

SEQ ID NO: 118 - protein, 4 aa, Artificial Sequence
  VH CDR3 of Ab9 (Table 3A)
RGGY

SEQ ID NO: 119 - protein, 9 aa, Artificial Sequence
  VH CDR3 of Ab10 (Table 3A)
GRGSTAFDI

SEQ ID NO: 120 - protein, 11 aa, Artificial Sequence
  VH CDR3 of Ab11, Ab67 and Ab123
KKMATFAAFDI

SEQ ID NO: 121 - protein, 8 aa, Artificial Sequence
  VH CDR3 of Ab12 (Table 3A)
SSGYHIDY

SEQ ID NO: 122 - protein, 30 aa, Artificial Sequence
  VH FR1 of Ab1 (Table 4A)
EVQLLESGGGLVQPGGSLRLSCAASGFTFS

SEQ ID NO: 123 - protein, 30 aa, Artificial Sequence; identical to SEQ ID NO: 122
  VH FR1 of Ab2 (Table 4A)

SEQ ID NO: 124 - protein, 30 aa, Artificial Sequence; identical to SEQ ID NO: 122
  VH FR1 of Ab3 (Table 4A)

SEQ ID NO: 125 - protein, 30 aa, Artificial Sequence; identical to SEQ ID NO: 122
  VH FR1 of Ab4 (Table 4A)

SEQ ID NO: 126 - protein, 30 aa, Artificial Sequence; identical to SEQ ID NO: 122
  VH FR1 of Ab5 (Table 4A)

SEQ ID NO: 127 - protein, 30 aa, Artificial Sequence; identical to SEQ ID NO: 122
  VH FR1 of Ab6 (Table 4A)

SEQ ID NO: 128 - protein, 30 aa, Artificial Sequence; identical to SEQ ID NO: 122
  VH FR1 of Ab7 (Table 4A)

SEQ ID NO: 129 - protein, 30 aa, Artificial Sequence; identical to SEQ ID NO: 122
  VH FR1 of Ab8 (Table 4A)

SEQ ID NO: 130 - protein, 30 aa, Artificial Sequence; identical to SEQ ID NO: 122
  VH FR1 of Ab9 (Table 4A)

SEQ ID NO: 131 - protein, 30 aa, Artificial Sequence; identical to SEQ ID NO: 122
  VH FR1 of Ab10 (Table 4A)

SEQ ID NO: 132 - protein, 30 aa, Artificial Sequence; identical to SEQ ID NO: 122
  VH FR1 of Ab11 (Table 4A)

SEQ ID NO: 133 - protein, 30 aa, Artificial Sequence; identical to SEQ ID NO: 122
  VH FR1 of Ab12 (Table 4A)

SEQ ID NO: 134 - protein, 14 aa, Artificial Sequence
  VH FR2 of Ab1 (Table 4A)
WVRQAPGKGLEWVS

SEQ ID NO: 135 - protein, 14 aa, Artificial Sequence
  VH FR2 of Ab2 (Table 4A)
WVRQAPGKGLEWVS

SEQ ID NO: 136 - protein, 14 aa, Artificial Sequence
  VH FR2 of Ab3 (Table 4A)
WVRQAPGKGLEWVS

SEQ ID NO: 137 - protein, 14 aa, Artificial Sequence
  VH FR2 of Ab4 (Table 4A)
WVRQAPGKGLEWVS

SEQ ID NO: 138 - protein, 14 aa, Artificial Sequence
  VH FR2 of Ab5 (Table 4A)
WVRQAPGKGLEWVS

SEQ ID NO: 139 - protein, 14 aa, Artificial Sequence
  VH FR2 of Ab6 (Table 4A)
WVRQAPGKGLEWVS

SEQ ID NO: 140 - protein, 14 aa, Artificial Sequence
  VH FR2 of Ab7 (Table 4A)
WVRQAPGKGLEWVS

SEQ ID NO: 141 - protein, 14 aa, Artificial Sequence
  VH FR2 of Ab8 (Table 4A)
WVRQAPGKGLEWVS

SEQ ID NO: 142 - protein, 14 aa, Artificial Sequence
  VH FR2 of Ab9 (Table 4A)
WVRQAPGKGLEWVS

SEQ ID NO: 143 - protein, 14 aa, Artificial Sequence
  VH FR2 of Ab10 (Table 4A)
WVRQAPGKGLEWVS

SEQ ID NO: 144 - protein, 14 aa, Artificial Sequence
  VH FR2 of Ab11 (Table 4A)
WVRQAPGKGLEWVS

SEQ ID NO: 145 - protein, 14 aa, Artificial Sequence
  VH FR2 of Ab12 (Table 4A)
WVRQAPGKGLEWVS

SEQ ID NO: 146 - protein, 32 aa, Artificial Sequence
  VH FR3 of Ab1 (Table 4A)
RFTISRDNSKNTLYLQMNSLRAEDTAVYYCAR

SEQ ID NO: 147 - protein, 32 aa, Artificial Sequence; identical to SEQ ID NO: 146
  VH FR3 of Ab2 (Table 4A)

SEQ ID NO: 148 - protein, 32 aa, Artificial Sequence; identical to SEQ ID NO: 146
  VH FR3 of Ab3 (Table 4A)

SEQ ID NO: 149 - protein, 32 aa, Artificial Sequence; identical to SEQ ID NO: 146
  VH FR3 of Ab4 (Table 4A)

SEQ ID NO: 150 - protein, 32 aa, Artificial Sequence
  VH FR3 of Ab5 (Table 4A)
RFTISRDNSKNTLYLQMNSLRAEDTAMYYCAR

SEQ ID NO: 151 - protein, 32 aa, Artificial Sequence; identical to SEQ ID NO: 146
  VH FR3 of Ab6 (Table 4A)

SEQ ID NO: 152 - protein, 32 aa, Artificial Sequence
  VH FR3 of Ab7 (Table 4A)
RFTISRDNSKNTLYLQMNSLRAEDTAVYYCTR

SEQ ID NO: 153 - protein, 32 aa, Artificial Sequence; identical to SEQ ID NO: 146
  VH FR3 of Ab8 (Table 4A)

SEQ ID NO: 154 - protein, 32 aa, Artificial Sequence
  VH FR3 of Ab9 (Table 4A)
RFTISRDNSKNTLYLQMNSLRAEDTAVYYCAT

SEQ ID NO: 155 - protein, 32 aa, Artificial Sequence; identical to SEQ ID NO: 146
  VH FR3 of Ab10 (Table 4A)

SEQ ID NO: 156 - protein, 32 aa, Artificial Sequence; identical to SEQ ID NO: 146
  VH FR3 of Ab11 (Table 4A)

SEQ ID NO: 157 - protein, 32 aa, Artificial Sequence; identical to SEQ ID NO: 146
  VH FR3 of Ab12 (Table 4A)

SEQ ID NO: 158 - protein, 11 aa, Artificial Sequence
  VH FR4 of Ab1 (Table 4A)
WGQGTMVTVSS

SEQ ID NO: 159 - protein, 11 aa, Artificial Sequence
  VH FR4 of Ab2 (Table 4A)
WGQGTLVTVSS

SEQ ID NO: 160 - protein, 11 aa, Artificial Sequence
  VH FR4 of Ab3 (Table 4A)
WGQGILVTVSS

SEQ ID NO: 161 - protein, 11 aa, Artificial Sequence
  VH FR4 of Ab4 (Table 4A)
WGQGTLVTVSS

SEQ ID NO: 162 - protein, 11 aa, Artificial Sequence
  VH FR4 of Ab5 (Table 4A)
WGQGTTVTVSS

SEQ ID NO: 163 - protein, 11 aa, Artificial Sequence
  VH FR4 of Ab6 (Table 4A)
WGQGTMVTVSS

SEQ ID NO: 164 - protein, 11 aa, Artificial Sequence
  VH FR4 of Ab7 (Table 4A)
WGQGTLVTVSS

SEQ ID NO: 165 - protein, 11 aa, Artificial Sequence
  VH FR4 of Ab8 (Table 4A)
WGQGTMVTVSS

SEQ ID NO: 166 - protein, 11 aa, Artificial Sequence
  VH FR4 of Ab9 (Table 4A)
WGQGTLVTVSS

SEQ ID NO: 167 - protein, 11 aa, Artificial Sequence
  VH FR4 of Ab10 (Table 4A)
WGQGTMVTVSS

SEQ ID NO: 168 - protein, 11 aa, Artificial Sequence
  VH FR4 of Ab11 (Table 4A)
WGQGTMVTVSS

SEQ ID NO: 169 - protein, 11 aa, Artificial Sequence
  VH FR4 of Ab12 (Table 4A)
WGQGTLVTVSS

SEQ ID NO: 170 - protein, 246 aa, Artificial Sequence
  KIT D4/D5 Antigen (Fig 2A)
MRGARGAWDFLCVLLLLLRVQTGSSQPSVSPGEVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKEQIHPHHHHHH

SEQ ID NO: 171 - protein, 208 aa, Artificial Sequence
  M1-E33 + V308-H515 (Fig 2A)
VDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKEQIHPH

SEQ ID NO: 172 - protein, 144 aa, Artificial Sequence
  KIT D4 Antigen (Fig 2B)
MRGARGAWDFLCVLLLLLRVQTGSSQPSVSPGEVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKHHHHHH

SEQ ID NO: 173 - protein, 105 aa, Artificial Sequence
  V308-K412 (Fig 2B)
VDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTK

SEQ ID NO: 174 - protein, 144 aa, Artificial Sequence
  KIT D5 Antigen (Fig 2C)
MRGARGAWDFLCVLLLLLRVQTGSSQPSVSPGENTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKEQIHPHHHHHH

SEQ ID NO: 175 - protein, 110 aa, Artificial Sequence
  N410-H515 (Fig 2C)
NTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPH

SEQ ID NO: 176 - protein, 108 aa, Artificial Sequence
  VL chain region of anti-KIT antibodies Ab1 (Fig. 3)
QDIQMTQSPTSLSAFVGDRVTITCQASQDIGNYLNWYQQKSGEPPKLLVYDASFLKKGVPSRFSGSGSGTQYFLTIYSLQPEDFATYFCQHSDNLSVTFGGGTKVEVK

SEQ ID NO: 177 - protein, 107 aa, Artificial Sequence
  VL chain region of anti-KIT antibodies Ab2 (Fig. 3)
QDIQMTQSPATLSLSPGERATLYCRASQSVSNYVAWYQQRPGQAPRLLMYDTSKRATGIPARFSGSGSGTDFTLTISSLEPEDFAVYYCQQRSYWFAFGGGTKVEIK

SEQ ID NO: 178 - protein, 110 aa, Artificial Sequence
  VL chain region of anti-KIT antibodies Ab3 (Fig. 3)
QSALTQPPSASGTPGQRVTISCSGSSSNIGNNYVYWYQQLPGTAPKLLIYRSNQRPSGVPDRFSGSKSGASASLAISGLRSEDEADYYCAAWDDSLIGPVFGGGTKLTVL

SEQ ID NO: 179 - protein, 109 aa, Artificial Sequence
  VL chain region of anti-KIT antibodies Ab4 (Fig. 3)
QDIQMTQSPSSLSASVGDRVAITCRASQSIDTYLNWYQQKPGKAPKLLIYAASKLEDGVPSRFSGSGTGTDFTLTIRSLQPEDFASYFCQQSYSSPGITFGPGTKVEIK

SEQ ID NO: 180 - protein, 109 aa, Artificial Sequence; identical to SEQ ID NO: 179
  VL chain region of anti-KIT antibodies Ab5 (Fig. 3)

SEQ ID NO: 181 - protein, 110 aa, Artificial Sequence
  VL chain region of anti-KIT antibodies Ab6 (Fig. 3)
QDIQMTQSPATLSLYPGERATLSCRASQSVGSSYLAWYQQKPGQAPRLLIYDASSRSSGIPDRFSGSGSGTDFTLTITRLEPEDFAVYYCQQRSNRPPWTFGQGTKVEIK

SEQ ID NO: 182 - protein, 109 aa, Artificial Sequence; identical to SEQ ID NO: 179
  VL chain region of anti-KIT antibodies Ab7 (Fig. 3)

SEQ ID NO: 183 - protein, 109 aa, Artificial Sequence
  VL chain region of anti-KIT antibodies Ab8 (Fig. 3)
QDIQMTQSPVTLSLSPGDRATLSCRASQSVSFNLAWYQHKPGQAPRLLMFDASTRAIGTPDRFSGSGSGTEFTLTISSLQSEDSAVYYCQQYNYPASYTFGQGTKLEIS

SEQ ID NO: 184 - protein, 108 aa, Artificial Sequence
  VL chain region of anti-KIT antibodies Ab9 (Fig. 3)
QDIQMTQSPATLSLSPGERATLSCRASQSVSSYLAWYQQKPGQAPRLLIYDASNRATGIPARFSGSGSGTDFTLTISSLEPEDFAVYYCQQRSNGGITFGQGTRLEIK

SEQ ID NO: 185 - protein, 109 aa, Artificial Sequence
  VL chain region of anti-KIT antibodies Ab10 (Fig. 3)
QDIQMTQSPDSLSVSPGERATLSCRASQSVRGNLAWYQQKPGQAPRLLIHGASTRATDIPTRFSGSGSGTDFTLTISSMQSEDFAVYYCQQYDSWPPFTFGPGTKVDIK

SEQ ID NO: 186 - protein, 110 aa, Artificial Sequence
  VL chain region of anti-KIT antibodies Ab11 (Fig. 3)
QDIQMTQSPGTLSLSPGERATLSCRASQSVSSSYLAWYQQKPGQAPRLLIYGASSRATGIPDRFSGSGSGTDFTLTISRLEPEDFAVYYCQQYGSSRTWTFGQGTKVEIK

SEQ ID NO: 187 - protein, 110 aa, Artificial Sequence
  VL chain region of anti-KIT antibodies Ab12 (Fig. 3)
QDIQMTQSPGTLSLSPGERATLYCRASQSVKSNYLAWYQQKPGQAPRLLIYGASTRATDIPDRFSGSGSGTDFTLTISRLEPEDFAVYYCQQYGSSLSYTFGQGTKLEIK

SEQ ID NO: 188 - protein, 122 aa, Artificial Sequence
  VH chain region of anti-KIT antibodies Ab1 (Fig. 4)
EVQLLESGGGLVQPGGSLRLSCAASGFTFSNYLMSWVRQAPGKGLEWVSSIVPSGGFTHYADSVKGRFTISRDNSKNTLYLQMNSLRAEDTAVYYCARLQTGSWRVHAFDIWGQGTMVTVSS

SEQ ID NO: 189 - protein, 120 aa, Artificial Sequence
  VH chain region of anti-KIT antibodies Ab2 (Fig. 4)
EVQLLESGGGLVQPGGSLRLSCAASGFTFSNYKMGWVRQAPGKGLEWVSWIYPSGGGTFQADSVKGRFTISRDNSKNTLYLQMNSLRAEDTAVYYCARVRVGSTGNFDYWGQGTLVTVSS

SEQ ID NO: 190 - protein, 120 aa, Artificial Sequence
  VH chain region of anti-KIT antibodies Ab3 (Fig. 4)
EVQLLESGGGLVQPGGSLRLSCAASGFTFSPYNMMWVRQAPGKGLEWVSRISPSGGDTHYADSVKGRFTISRDNSKNTLYLQMNSLRAEDTAVYYCARGSRSSWSTPYYWGQGILVTVSS

SEQ ID NO: 191 - protein, 126 aa, Artificial Sequence
  VH chain region of anti-KIT antibodies Ab4 (Fig. 4)
EVQLLESGGGLVQPGGSLRLSCAASGFTFSWYWMYWVRQAPGKGLEWVSGIYPSGGFTSYADSVKGRFTISRDNSKNTLYLQMNSLRAEDTAVYYCARSYYYGSGSYYNSASFDYWGQGTLVTVSS

SEQ ID NO: 192 - protein, 117 aa, Artificial Sequence
  VH chain region of anti-KIT antibodies Ab5 (Fig. 4)
EVQLLESGGGLVQPGGSLRLSCAASGFTFSSYPMAWVRQAPGKGLEWVSVISSSGGYTPYADSVKGRFTISRDNSKNTLYLQMNSLRAEDTAMYYCARRYGGGMDVWGQGTTVTVSS

SEQ ID NO: 193 - protein, 117 aa, Artificial Sequence
  VH chain region of anti-KIT antibodies Ab6 (Fig. 4)
EVQLLESGGGLVQPGGSLRLSCAASGFTFSYYSMGWVRQAPGKGLEWVSVIRPSGGHTHYADSVKGRFTISRDNSKNTLYLQMNSLRAEDTAVYYCARLATNAFDIWGQGTMVTVSS

SEQ ID NO: 194 - protein, 117 aa, Artificial Sequence
  VH chain region of anti-KIT antibodies Ab7 (Fig. 4)
EVQLLESGGGLVQPGGSLRLSCAASGFTFSRYPMNWVRQAPGKGLEWVSGIRSSGGATSYADSVKGRFTISRDNSKNTLYLQMNSLRAEDTAVYYCTRSFGRAFDYWGQGTLVTVSS

SEQ ID NO: 195 - protein, 127 aa, Artificial Sequence
  VH chain region of anti-KIT antibodies Ab8 (Fig. 4)
EVQLLESGGGLVQPGGSLRLSCAASGFTFSNYKMQWVRQAPGKGLEWVSYISSSGGYTSYADSVKGRFTISRDNSKNTLYLQMNSLRAEDTAVYYCARVAHQYYDFWSGYYGAFDIWGQGTMVTVSS

SEQ ID NO: 196 - protein, 113 aa, Artificial Sequence
  VH chain region of anti-KIT antibodies Ab9 (Fig. 4)
EVQLLESGGGLVQPGGSLRLSCAASGFTFSNYLMQWVRQAPGKGLEWVSGIVPSGGSTVYADSVKGRFTISRDNSKNTLYLQMNSLRAEDTAVYYCATRGGYWGQGTLVTVSS

SEQ ID NO: 197 - protein, 118 aa, Artificial Sequence
  VH chain region of anti-KIT antibodies Ab10 (Fig. 4)
EVQLLESGGGLVQPGGSLRLSCAASGFTFSIYSMMWVRQAPGKGLEWVSWIVSSGGQTYYADSVKGRFTISRDNSKNTLYLQMNSLRAEDTAVYYCARGRGSTAFDIWGQGTMVTVSS

SEQ ID NO: 198 - protein, 120 aa, Artificial Sequence
  VH chain region of anti-KIT antibodies Ab11 (Fig. 4)
EVQLLESGGGLVQPGGSLRLSCAASGFTFSNYRMMWVRQAPGKGLEWVSGIYPSGGGTFYADSVKGRFTISRDNSKNTLYLQMNSLRAEDTAVYYCARKKMATFAAFDIWGQGTMVTVSS

SEQ ID NO: 199 - protein, 117 aa, Artificial Sequence
  VH chain region of anti-KIT antibodies Ab12 (Fig. 4)
EVQLLESGGGLVQPGGSLRLSCAASGFTFSFYKMHWVRQAPGKGLEWVSGIWPSGGYTEYADSVKGRFTISRDNSKNTLYLQMNSLRAEDTAVYYCARSSGYHIDYWGQGTLVTVSS

SEQ ID NO: 200 - protein, 12 aa, Artificial Sequence
  VL CDR1 of Ab13 (Table 1B)
RASQSVRSNYLA

SEQ ID NO: 201 - protein, 11 aa, Artificial Sequence
  VL CDR1 of Ab14 (Table 1B)
RASQGVSTYLA

SEQ ID NO: 202 - protein, 12 aa, Artificial Sequence
  VL CDR1 of Ab15 (Table 1B)
RASQSVSSSYLA

SEQ ID NO: 203 - protein, 12 aa, Artificial Sequence
  VL CDR1 of Ab16 (Table 1B)
RASQSVSSSYLA

SEQ ID NO: 204 - protein, 11 aa, Artificial Sequence
  VL CDR1 of Ab17 (Table 1B)
RASQNIDNYLN

SEQ ID NO: 205 - protein, 13 aa, Artificial Sequence
  VL CDR1 of Ab18 and Ab189 (Table 1B)
SGSSSNIGSNYVY

SEQ ID NO: 206 - protein, 12 aa, Artificial Sequence
  VL CDR1 of Ab19 (Table 1B)
RASQSFRSSYLA

SEQ ID NO: 207 - protein, 11 aa, Artificial Sequence
  VL CDR1 of Ab20, and Ab150
RASQSVNIYLA

SEQ ID NO: 208 - protein, 7 aa, Artificial Sequence
  VL CDR2 of Ab13 (Table 1B)
GASSRAT

SEQ ID NO: 209 - protein, 7 aa, Artificial Sequence
  VL CDR2 of Ab14 (Table 1B)
GASTRAT

SEQ ID NO: 210 - protein, 7 aa, Artificial Sequence
  VL CDR2 of Ab15 (Table 1B)
GASSRAT

SEQ ID NO: 211 - protein, 7 aa, Artificial Sequence
  VL CDR2 of Ab16 (Table 1B)
GASSRAT

SEQ ID NO: 212 - protein, 7 aa, Artificial Sequence
  VL CDR2 of Ab17, Ab39, Ab44, Ab59, Ab81, Ab110, Ab116, Ab126, Ab151, Ab154, Ab155, Ab156 and Ab188
AASSLQS

SEQ ID NO: 213 - protein, 7 aa, Artificial Sequence
  VL CDR2 of Ab18 (Table 1B)
RNNKRPS

SEQ ID NO: 214 - protein, 7 aa, Artificial Sequence
  VL CDR2 of Ab19 (Table 1B)
DTSTRAT

SEQ ID NO: 215 - protein, 5 aa, Artificial Sequence
  VL CDR2 of Ab20 and Ab150
DASNR

SEQ ID NO: 216 - protein, 9 aa, Artificial Sequence
  VL CDR3 of Ab13, Ab331, and Ab119
QQRSNWPIT

SEQ ID NO: 217 - protein, 9 aa, Artificial Sequence
  VL CDR3 of Ab14 (Table 1B)
QQRRSWPPT

SEQ ID NO: 218 - protein, 10 aa, Artificial Sequence
  VL CDR3 of Ab15 and Ab68
QQYGSSPPYT

SEQ ID NO: 219 - protein, 8 aa, Artificial Sequence
  VL CDR3 of Ab16 and Ab69
QQYGSSWT

SEQ ID NO: 220 - protein, 9 aa, Artificial Sequence
  VL CDR3 of Ab17 and Ab70
QQSYSNSRT

SEQ ID NO: 221 - protein, 11 aa, Artificial Sequence
  VL CDR3 of Ab18 (Table 1B)
AAWDDSLNGVI

SEQ ID NO: 222 - protein, 11 aa, Artificial Sequence
  VL CDR3 of Ab19 (Table 1B)
QQYGSSPPMYT

SEQ ID NO: 223 - protein, 11 aa, Artificial Sequence
  VL CDR3 of Ab20 and Ab150
QQRDSWPPRLS

SEQ ID NO: 224 - protein, 24 aa, Artificial Sequence
  VL FR1 of Ab13 (Table 2B)
QDIQMTQSPGTLSLSPGERATLSC

SEQ ID NO: 225 - protein, 24 aa, Artificial Sequence
  VL FR1 of Ab14 (Table 2B)
QDIQMTQSPATLSVSPGERATLSC

SEQ ID NO: 226 - protein, 24 aa, Artificial Sequence
  VL FR1 of Ab15 (Table 2B)
QDIQMTQSPGTLSLSPGERATLSC

SEQ ID NO: 227 - protein, 24 aa, Artificial Sequence
  VL FR1 of Ab16 (Table 2B)
QDIQMTQSPGTLSLSPGERATLSC

SEQ ID NO: 228 - protein, 24 aa, Artificial Sequence
  VL FR1 of Ab17 (Table 2B)
QDIQMTQSPSSLSASVGDRVTVTC

SEQ ID NO: 229 - protein, 22 aa, Artificial Sequence
  VL FR1 of Ab18 (Table 2B)
QSALTQPPSTSGTPGQRVTISC

SEQ ID NO: 230 - protein, 24 aa, Artificial Sequence
  VL FR1 of Ab19 (Table 2B)
QDIQMTQSPGTLSLSPGERATLSC

SEQ ID NO: 231 - protein, 24 aa, Artificial Sequence
  VL FR1 of Ab20 (Table 2B)
QDIQMTQSPATLSLSPGERATLSC

SEQ ID NO: 232 - protein, 15 aa, Artificial Sequence
  VL FR2 of Ab13 (Table 2B)
WYQQKPGQAPRLLIY

SEQ ID NO: 233 - protein, 15 aa, Artificial Sequence
  VL FR2 of Ab14 (Table 2B)
WYQQKPGQAPRLLIY

SEQ ID NO: 234 - protein, 15 aa, Artificial Sequence
  VL FR2 of Ab15 (Table 2B)
WYQQKPGQAPRLLIY

SEQ ID NO: 235 - protein, 15 aa, Artificial Sequence
  VL FR2 of Ab16 (Table 2B)
WYQQKPGQAPRLLIY

SEQ ID NO: 236 - protein, 15 aa, Artificial Sequence
  VL FR2 of Ab17 (Table 2B)
WYQQRPGKAPELLIY

SEQ ID NO: 237 - protein, 15 aa, Artificial Sequence
  VL FR2 of Ab18 (Table 2B)
WYQQLPGTAPKVLIY

SEQ ID NO: 238 - protein, 15 aa, Artificial Sequence
  VL FR2 of Ab19 (Table 2B)
WYQQKPGQAPRLLIY

SEQ ID NO: 239 - protein, 15 aa, Artificial Sequence
  VL FR2 of Ab20 (Table 2B)
WYQQRPGQAPRLLIY

SEQ ID NO: 240 - protein, 32 aa, Artificial Sequence; identical to SEQ ID NO: 72
  VL FR3 of Ab13 (Table 2B)

SEQ ID NO: 241 - protein, 32 aa, Artificial Sequence
  VL FR3 of Ab14 (Table 2B)
GIPARFSGSGSGTDFTLTINSLELEDFAVYYC

SEQ ID NO: 242 - protein, 32 aa, Artificial Sequence; identical to SEQ ID NO: 72
  VL FR3 of Ab15 (Table 2B)

SEQ ID NO: 243 - protein, 32 aa, Artificial Sequence; identical to SEQ ID NO: 72
  VL FR3 of Ab16 (Table 2B)

SEQ ID NO: 244 - protein, 32 aa, Artificial Sequence
  VL FR3 of Ab17 (Table 2B)
GVPSRFSGSGSGTDFTLTISSLQPEDFATYYC

SEQ ID NO: 245 - protein, 32 aa, Artificial Sequence
  VL FR3 of Ab18 (Table 2B)
GVPDRFSGSKSGTSASLAISGLRSEDEADYYC

SEQ ID NO: 246 - protein, 32 aa, Artificial Sequence; identical to SEQ ID NO: 72
  VL FR3 of Ab19 (Table 2B)

SEQ ID NO: 247 - protein, 34 aa, Artificial Sequence
  VL FR3 of Ab20 (Table 2B)
AIGVPARFSGTGSGTDFTLTISSLEPEDSAVYYC

SEQ ID NO: 248 - protein, 10 aa, Artificial Sequence
  VL FR4 of Ab13 (Table 2B)
FGQGTRLEIK

SEQ ID NO: 249 - protein, 10 aa, Artificial Sequence
  VL FR4 of Ab14 (Table 2B)
FGQGTKVEIK

SEQ ID NO: 250 - protein, 10 aa, Artificial Sequence
  VL FR4 of Ab15 (Table 2B)
FGQGTKLEIK

SEQ ID NO: 251 - protein, 10 aa, Artificial Sequence
  VL FR4 of Ab16 (Table 2B)
FGQGTKVEIK

SEQ ID NO: 252 - protein, 10 aa, Artificial Sequence
  VL FR4 of Ab17 (Table 2B)
FGQGTKVEIK

SEQ ID NO: 253 - protein, 10 aa, Artificial Sequence
  VL FR4 of Ab18 (Table 2B)
FGGGTRLTVL

SEQ ID NO: 254 - protein, 10 aa, Artificial Sequence
  VL FR4 of Ab19 (Table 2B)
FGQGTKLEIK

SEQ ID NO: 255 - protein, 10 aa, Artificial Sequence
  VL FR4 of Ab20 (Table 2B)
FGGGTKVEIK

SEQ ID NO: 256 - protein, 5 aa, Artificial Sequence
  VH CDR1 of Ab13 and Ab78
NYTMH

SEQ ID NO: 257 - protein, 5 aa, Artificial Sequence
  VH CDR1 of Ab14 (Table 3B)
RYAMW

SEQ ID NO: 258 - protein, 5 aa, Artificial Sequence
  VH CDR1 of Ab15 and Ab68
NYTMM

SEQ ID NO: 259 - protein, 5 aa, Artificial Sequence
  VH CDR1 of Ab16 and Ab69
QYVMF

SEQ ID NO: 260 - protein, 5 aa, Artificial Sequence
  VH CDR1 of Ab17 (Table 3B)
NYTMI

SEQ ID NO: 261 - protein, 5 aa, Artificial Sequence
  VH CDR1 of Ab18 (Table 3B)
WYAMW

SEQ ID NO: 262 - protein, 5 aa, Artificial Sequence
  VH CDR1 of Ab19 (Table 3B)
NYQMQ

SEQ ID NO: 263 - protein, 5 aa, Artificial Sequence
  VH CDR1 of Ab20 and Ab150
RYGMA

SEQ ID NO: 264 - protein, 17 aa, Artificial Sequence
  VH CDR2 of Ab13 (Table 3B)
VIYPSGGMTKYADSVKG

SEQ ID NO: 265 - protein, 17 aa, Artificial Sequence
  VH CDR2 of Ab14 (Table 3B)
YIYSSGGQTGYADSVKG

SEQ ID NO: 266 - protein, 17 aa, Artificial Sequence
  VH CDR2 of Ab15 and Ab68
GIRPSGGGTMYADSVKG

SEQ ID NO: 267 - protein, 17 aa, Artificial Sequence
  VH CDR2 of Ab16 and Ab69
SIYPSGGHTLYADSVKG

SEQ ID NO: 268 - protein, 17 aa, Artificial Sequence
  VH CDR2 of Ab17 (Table 3B)
RIWPSGGGTDYADSVKG

SEQ ID NO: 269 - protein, 17 aa, Artificial Sequence
  VH CDR2 of Ab18 (Table 3B)
YISSSGGATRYADSVKG

SEQ ID NO: 270 - protein, 17 aa, Artificial Sequence
  VH CDR2 of Ab19 (Table 3B)
SIRPSGGSTQYADSVKG

SEQ ID NO: 271 - protein, 17 aa, Artificial Sequence
  VH CDR2 of Ab20 and Ab150
SIRPSGGYTAYADSVKG

SEQ ID NO: 272 - protein, 7 aa, Artificial Sequence
  VH CDR3 of Ab13 (Table 3B)
GSTTFDY

SEQ ID NO: 273 - protein, 7 aa, Artificial Sequence
  VH CDR3 of Ab14 (Table 3B)
GGVTFDL

SEQ ID NO: 274 - protein, 8 aa, Artificial Sequence
  VH CDR3 of Ab15 and Ab68
VVNHKFDN

SEQ ID NO: 275 - protein, 12 aa, Artificial Sequence
  VH CDR3 of Ab16 and Ab69
HARGYNRGYFDS

SEQ ID NO: 276 - protein, 10 aa, Artificial Sequence
  VH CDR3 of Ab17 (Table 3B)
RVGASRYFDL

SEQ ID NO: 277 - protein, 9 aa, Artificial Sequence
  VH CDR3 of Ab18 (Table 3B)
VLQYGAFDI

SEQ ID NO: 278 - protein, 8 aa, Artificial Sequence
  VH CDR3 of Ab19 (Table 3B)
GSSHAFDI

SEQ ID NO: 279 - protein, 13 aa, Artificial Sequence
  VH CDR3 of Ab20 and Ab150
LPARFDYYYGLDV

SEQ ID NO: 280 - protein, 30 aa, Artificial Sequence; identical to SEQ ID NO: 122
  VH FR1 of Ab13 (Table 4B)

SEQ ID NO: 281 - protein, 30 aa, Artificial Sequence; identical to SEQ ID NO: 122
  VH FR1 of Ab14 (Table 4B)

SEQ ID NO: 282 - protein, 30 aa, Artificial Sequence; identical to SEQ ID NO: 122
  VH FR1 of Ab15 (Table 4B)

SEQ ID NO: 283 - protein, 30 aa, Artificial Sequence; identical to SEQ ID NO: 122
  VH FR1 of Ab16 (Table 4B)

SEQ ID NO: 284 - protein, 30 aa, Artificial Sequence; identical to SEQ ID NO: 122
  VH FR1 of Ab17 (Table 4B)

SEQ ID NO: 285 - protein, 30 aa, Artificial Sequence; identical to SEQ ID NO: 122
  VH FR1 of Ab18 (Table 4B)

SEQ ID NO: 286 - protein, 30 aa, Artificial Sequence; identical to SEQ ID NO: 122
  VH FR1 of Ab19 (Table 4B)

SEQ ID NO: 287 - protein, 30 aa, Artificial Sequence; identical to SEQ ID NO: 122
  VH FR1 of Ab20 (Table 4B)

SEQ ID NO: 288 - protein, 14 aa, Artificial Sequence
  VH FR2 of Ab13 (Table 4B)
WVRQAPGKGLEWVS

SEQ ID NO: 289 - protein, 14 aa, Artificial Sequence
  VH FR2 of Ab14 (Table 4B)
WVRQAPGKGLEWVS

SEQ ID NO: 290 - protein, 14 aa, Artificial Sequence
  VH FR2 of Ab15 (Table 4B)
WVRQAPGKGLEWVS

SEQ ID NO: 291 - protein, 14 aa, Artificial Sequence
  VH FR2 of Ab16 (Table 4B)
WVRQAPGKGLEWVS

SEQ ID NO: 292 - protein, 14 aa, Artificial Sequence
  VH FR2 of Ab17 (Table 4B)
WVRQAPGKGLEWVS

SEQ ID NO: 293 - protein, 14 aa, Artificial Sequence
  VH FR2 of Ab18 (Table 4B)
WVRQAPGKGLEWVS

SEQ ID NO: 294 - protein, 14 aa, Artificial Sequence
  VH FR2 of Ab19 (Table 4B)
WVRQAPGKGLEWVS

SEQ ID NO: 295 - protein, 14 aa, Artificial Sequence
  VH FR2 of Ab20 (Table 4B)
WVRQAPGKGLEWVS

SEQ ID NO: 296 - protein, 32 aa, Artificial Sequence; identical to SEQ ID NO: 146
  VH FR3 of Ab13 (Table 4B)

SEQ ID NO: 297 - protein, 32 aa, Artificial Sequence; identical to SEQ ID NO: 146
  VH FR3 of Ab14 (Table 4B)

SEQ ID NO: 298 - protein, 32 aa, Artificial Sequence
  VH FR3 of Ab15 (Table 4B)
RFTISRDNSKNTLYLQMNSLRAEDTAVYYCAS

SEQ ID NO: 299 - protein, 32 aa, Artificial Sequence
  VH FR3 of Ab16 (Table 4B)
RFTISRDNSKNTLYLQMNSLRAEDTAVYYCAK

SEQ ID NO: 300 - protein, 32 aa, Artificial Sequence
  VH FR3 of Ab17 (Table 4B)
RFTISRDNSKNTLYLQMNSLRAEDTATYYCAR

SEQ ID NO: 301 - protein, 32 aa, Artificial Sequence; identical to SEQ ID NO: 154
  VH FR3 of Ab18 (Table 4B)

SEQ ID NO: 302 - protein, 32 aa, Artificial Sequence; identical to SEQ ID NO: 146
  VH FR3 of Ab19 (Table 4B)

SEQ ID NO: 303 - protein, 32 aa, Artificial Sequence; identical to SEQ ID NO: 146
  VH FR3 of Ab20 (Table 4B)

SEQ ID NO: 304 - protein, 11 aa, Artificial Sequence
  VH FR4 of Ab13 (Table 4B)
WGQGTLVTVSS

SEQ ID NO: 305 - protein, 11 aa, Artificial Sequence
  VH FR4 of Ab14 (Table 4B)
WGRGTLVTVSS

SEQ ID NO: 306 - protein, 11 aa, Artificial Sequence
  VH FR4 of Ab15 (Table 4B)
WGQGTLVTVSS

SEQ ID NO: 307 - protein, 11 aa, Artificial Sequence
  VH FR4 of Ab16 (Table 4B)
WGRGTLVTVSS

SEQ ID NO: 308 - protein, 11 aa, Artificial Sequence
  VH FR4 of Ab17 (Table 4B)
WGRGTLVTVSS

SEQ ID NO: 309 - protein, 11 aa, Artificial Sequence
  VH FR4 of Ab18 (Table 4B)
WGQGTMVTVSS

SEQ ID NO: 310 - protein, 11 aa, Artificial Sequence
  VH FR4 of Ab19 (Table 4B)
WGQGTMVTVSS

SEQ ID NO: 311 - protein, 11 aa, Artificial Sequence
  VH FR4 of Ab20 (Table 4B)
WGQGTTVTVSS

SEQ ID NO: 312 - protein, 109 aa, Artificial Sequence
  Anti-KIT antibody Ab13 VL domain (Fig. 5M)
QDIQMTQSPGTLSLSPGERATLSCRASQSVRSNYLAWYQQKPGQAPRLLIYGASSRATGIPDRFSGSGSGTDFTLTISRLEPEDFAVYYCQQRSNWPITFGQGTRLEIK

SEQ ID NO: 313 - protein, 108 aa, Artificial Sequence
  Anti-KIT antibody Ab14 VL domain (Fig. 5N)
QDIQMTQSPATLSVSPGERATLSCRASQGVSTYLAWYQQKPGQAPRLLIYGASTRATGIPARFSGSGSGTDFTLTINSLELEDFAVYYCQQRRSWPPTFGQGTKVEIK

SEQ ID NO: 314 - protein, 110 aa, Artificial Sequence
  Anti-KIT antibody Ab15 VL domain (Fig. 5O)
QDIQMTQSPGTLSLSPGERATLSCRASQSVSSSYLAWYQQKPGQAPRLLIYGASSRATGIPDRFSGSGSGTDFTLTISRLEPEDFAVYYCQQYGSSPPYTFGQGTKLEIK

SEQ ID NO: 315 - protein, 108 aa, Artificial Sequence
  Anti-KIT antibody Ab16 VL domain (Fig. 5P)
QDIQMTQSPGTLSLSPGERATLSCRASQSVSSSYLAWYQQKPGQAPRLLIYGASSRATGIPDRFSGSGSGTDFTLTISRLEPEDFAVYYCQQYGSSWTFGQGTKVEIK

SEQ ID NO: 316 - protein, 108 aa, Artificial Sequence
  Anti-KIT antibody Ab17 VL domain (Fig. 5Q)
QDIQMTQSPSSLSASVGDRVTVTCRASQNIDNYLNWYQQRPGKAPELLIYAASSLQSGVPSRFSGSGSGTDFTLTISSLQPEDFATYYCQQSYSNSRTFGQGTKVEIK

SEQ ID NO: 317 - protein, 110 aa, Artificial Sequence
  Anti-KIT antibody Ab18 VL domain (Fig. 5R)
QSALTQPPSTSGTPGQRVTISCSGSSSNIGSNYVYWYQQLPGTAPKVLIYRNNKRPSGVPDRFSGSKSGTSASLAISGLRSEDEADYYCAAWDDSLNGVIFGGGTRLTVL

SEQ ID NO: 318 - protein, 111 aa, Artificial Sequence
  Anti-KIT antibody Ab19 VL domain (Fig. 5S)
QDIQMTQSPGTLSLSPGERATLSCRASQSFRSSYLAWYQQKPGQAPRLLIYDTSTRATGIPDRFSGSGSGTDFTLTISRLEPEDFAVYYCQQYGSSPPMYTFGQGTKLEIK

SEQ ID NO: 319 - protein, 110 aa, Artificial Sequence
  Anti-KIT antibody Ab20 VL domain (Fig. 5T)
QDIQMTQSPATLSLSPGERATLSCRASQSVNIYLAWYQQRPGQAPRLLIYDASNRAIGVPARFSGTGSGTDFTLTISSLEPEDSAVYYCQQRDSWPPRLSFGGGTKVEIK

SEQ ID NO: 320 - protein, 116 aa, Artificial Sequence
  Anti-KIT antibody Ab13 VH domain (Fig. 5M)
EVQLLESGGGLVQPGGSLRLSCAASGFTFSNYTMHWVRQAPGKGLEWVSVIYPSGGMTKYADSVKGRFTISRDNSKNTLYLQMNSLRAEDTAVYYCARGSTTFDYWGQGTLVTVSS

SEQ ID NO: 321 - protein, 116 aa, Artificial Sequence
  Anti-KIT antibody Ab14 VH domain (Fig. 5N)
EVQLLESGGGLVQPGGSLRLSCAASGFTFSRYAMWWVRQAPGKGLEWVSYIYSSGGQTGYADSVKGRFTISRDNSKNTLYLQMNSLRAEDTAVYYCARGGVTFDLWGRGTLVTVSS

SEQ ID NO: 322 - protein, 117 aa, Artificial Sequence
  Anti-KIT antibody Ab15 VH domain (Fig. 5O)
EVQLLESGGGLVQPGGSLRLSCAASGFTFSNYTMMWVRQAPGKGLEWVSGIRPSGGGTMYADSVKGRFTISRDNSKNTLYLQMNSLRAEDTAVYYCASVVNHKFDNWGQGTLVTVSS

SEQ ID NO: 323 - protein, 121 aa, Artificial Sequence
  Anti-KIT antibody Ab16 VH domain (Fig. 5P)
EVQLLESGGGLVQPGGSLRLSCAASGFTFSQYVMFWVRQAPGKGLEWVSSIYPSGGHTLYADSVKGRFTISRDNSKNTLYLQMNSLRAEDTAVYYCAKHARGYNRGYFDSWGRGTLVTVSS

SEQ ID NO: 324 - protein, 119 aa, Artificial Sequence
  Anti-KIT antibody Ab17 VH domain (Fig. 5Q)
EVQLLESGGGLVQPGGSLRLSCAASGFTFSNYTMIWVRQAPGKGLEWVSRIWPSGGGTDYADSVKGRFTISRDNSKNTLYLQMNSLRAEDTATYYCARRVGASRYFDLWGRGTLVTVSS

SEQ ID NO: 325 - protein, 118 aa, Artificial Sequence
  Anti-KIT antibody Ab18 VH domain (Fig. 5R)
EVQLLESGGGLVQPGGSLRLSCAASGFTFSWYAMWWVRQAPGKGLEWVSYISSSGGATRYADSVKGRFTISRDNSKNTLYLQMNSLRAEDTAVYYCATVLQYGAFDIWGQGTMVTVSS

SEQ ID NO: 326 - protein, 117 aa, Artificial Sequence
  Anti-KIT antibody Ab19 VH domain (Fig. 5S)
EVQLLESGGGLVQPGGSLRLSCAASGFTFSNYQMQWVRQAPGKGLEWVSSIRPSGGSTQYADSVKGRFTISRDNSKNTLYLQMNSLRAEDTAVYYCARGSSHAFDIWGQGTMVTVSS

SEQ ID NO: 327 - protein, 122 aa, Artificial Sequence
  Anti-KIT antibody Ab20 VH domain (Fig. 5T)
EVQLLESGGGLVQPGGSLRLSCAASGFTFSRYGMAWVRQAPGKGLEWVSSIRPSGGYTAYADSVKGRFTISRDNSKNTLYLQMNSLRAEDTAVYYCARLPARFDYYYGLDVWGQGTTVTVSS

SEQ ID NO: 328 - protein, 107 aa, Artificial Sequence
  Anti-KIT antibody Ab21 VL domain (Fig. 5U)
DIQMTQSPTSLSAFVGDRVTITCQASQDIGNYLNWYQQKSGEPPKLLVYDASFLKKGVPSRFSGSGSGTQYFLTIYSLQPEDFATYFCQHSDSLSVTFGGGTKVEVK

SEQ ID NO: 329 - protein, 122 aa, Artificial Sequence; identical to SEQ ID NO: 188
  Anti-KIT antibody Ab21 VH domain (Fig. 5U)

SEQ ID NO: 330 - protein, 11 aa, Artificial Sequence
  VL CDR1 of Ab21 (Table 1B)
QASQDIGNYLN

SEQ ID NO: 331 - protein, 7 aa, Artificial Sequence
  VL CDR2 of Ab21 (Table 1B)
DASFLKK

SEQ ID NO: 332 - protein, 9 aa, Artificial Sequence
  VL CDR3 of Ab21 (Table 1B)
QHSDSLSVT

SEQ ID NO: 333 - protein, 23 aa, Artificial Sequence
  VL FR1 of Ab21 (Table 2B)
DIQMTQSPTSLSAFVGDRVTITC

SEQ ID NO: 334 - protein, 15 aa, Artificial Sequence
  VL FR2 of Ab21 (Table 2B)
WYQQKSGEPPKLLVY

SEQ ID NO: 335 - protein, 32 aa, Artificial Sequence; identical to SEQ ID NO: 62
  VL FR3 of Ab21 (Table 2B)

SEQ ID NO: 336 - protein, 10 aa, Artificial Sequence
  VL FR4 of Ab21 (Table 2B)
FGGGTKVEVK

SEQ ID NO: 337 - protein, 5 aa, Artificial Sequence
  VH CDR1 of Ab21 (Table 3B)
NYLMS

SEQ ID NO: 338 - protein, 17 aa, Artificial Sequence
  VH CDR2 of Ab21 (Table 3B)
SIVPSGGFTHYADSVKG

SEQ ID NO: 339 - protein, 13 aa, Artificial Sequence
  VH CDR3 of Ab21 (Table 3B)
LQTGSWRVHAFDI

SEQ ID NO: 340 - protein, 30 aa, Artificial Sequence; identical to SEQ ID NO: 122
  VH FR1 of Ab21 (Table 4B)

SEQ ID NO: 341 - protein, 14 aa, Artificial Sequence
  VH FR2 of Ab21 (Table 4B)
WVRQAPGKGLEWVS

SEQ ID NO: 342 - protein, 32 aa, Artificial Sequence; identical to SEQ ID NO: 146
  VH FR3 of Ab21 (Table 4B)

SEQ ID NO: 343 - protein, 11 aa, Artificial Sequence
  VH FR4 of Ab21 (Table 4B)
WGQGTMVTVSS

SEQ ID NO: 344 - protein, 107 aa, Artificial Sequence [sequence omitted]

SEQ ID NO: 345 - protein, 330 aa, Artificial Sequence [sequence omitted]

SEQ ID NO: 346 - protein, 214 aa, Artificial Sequence
  Light chain of Ab1 (Page 12, Claim 66)
DIQMTQSPTSLSAFVGDRVTITCQASQDIGNYLNWYQQKSGEPPKLLVYDASFLKKGVPSRFSGSGSGTQYFLTIYSLQPEDFATYFCQHSDNLSVTFGGGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 347 - protein, 452 aa, Artificial Sequence
  Heavy chain of Ab1 (Page 12, Claim 67)
EVQLLESGGGLVQPGGSLRLSCAASGFTFSNYLMSWVRQAPGKGLEWVSSIVPSGGFTHYADSVKGRFTISRDNSKNTLYLQMNSLRAEDTAVYYCARLQTGSWRVHAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 348 - protein, 214 aa, Artificial Sequence
  Light chain of Ab21 (Page 12-13, Claim 69)
DIQMTQSPTSLSAFVGDRVTITCQASQDIGNYLNWYQQKSGEPPKLLVYDASFLKKGVPSRFSGSGSGTQYFLTIYSLQPEDFATYFCQHSDSLSVTFGGGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 349 - protein, 452 aa, Artificial Sequence; identical to SEQ ID NO: 347
  Heavy chain of Ab21 (Page 13, Claim 70)

SEQ ID NO: 350 - protein, 107 aa, Artificial Sequence
  Ab1 VL domain (Fig. 5A)
DIQMTQSPTSLSAFVGDRVTITCQASQDIGNYLNWYQQKSGEPPKLLVYDASFLKKGVPSRFSGSGSGTQYFLTIYSLQPEDFATYFCQHSDNLSVTFGGGTKVEVK

SEQ ID NO: 351 - protein, 12 aa, Artificial Sequence
  VL CDR1 of Ab24 and Ab146
RASQSVGSNFLA

SEQ ID NO: 352 - protein, 11 aa, Artificial Sequence
  VL CDR1 of Ab25 (Table 1C)
RASQNINSWLA

SEQ ID NO: 353 - protein, 11 aa, Artificial Sequence
  VL CDR1 of Ab26 (Table 1C)
RASQGIANYLS

SEQ ID NO: 354 - protein, 12 aa, Artificial Sequence
  VL CDR1 of Ab27 (Table 1C)
RASQTVSNNFLA

SEQ ID NO: 355 - protein, 11 aa, Artificial Sequence
  VL CDR1 of Ab28 (Table 1C)
RASQSISTWVA

SEQ ID NO: 356 - protein, 11 aa, Artificial Sequence
  VL CDR1 of Ab29 (Table 1C)
RASQSIRTNLA

SEQ ID NO: 357 - protein, 11 aa, Artificial Sequence
  VL CDR1 of Ab30 (Table 1C)
RASQSISRWLA

SEQ ID NO: 358 - protein, 11 aa, Artificial Sequence
  VL CDR1 of Ab32 (Table 1C)
RASQSVSTSLA

SEQ ID NO: 359 - protein, 11 aa, Artificial Sequence
  VL CDR1 of Ab33 and Ab140
RGDRLRTYYSS

SEQ ID NO: 360 - protein, 11 aa, Artificial Sequence
  VL CDR1 of Ab34 (Table 1C)
RASQGIYTYLV

SEQ ID NO: 361 - protein, 11 aa, Artificial Sequence
  VL CDR1 of Ab35 (Table 1C)
RASRSISDWLA

SEQ ID NO: 362 - protein, 16 aa, Artificial Sequence
  VL CDR1 of Ab36 (Table 1C)
RASQSVSTNYLGTSLA

SEQ ID NO: 363 - protein, 11 aa, Artificial Sequence
  VL CDR1 of Ab39 (Table 1C)
RASQSISKYLN

SEQ ID NO: 364 - protein, 11 aa, Artificial Sequence
  VL CDR1 of Ab40 (Table 1C)
RASESIGFWVA

SEQ ID NO: 365 - protein, 11 aa, Artificial Sequence
  VL CDR1 of Ab42 (Table 1C)
RASQSVSKYLN

SEQ ID NO: 366 - protein, 11 aa, Artificial Sequence
  VL CDR1 of Ab43 (Table 1C)
RASQSVRGRLA

SEQ ID NO: 367 - protein, 11 aa, Artificial Sequence
  VL CDR1 of Ab44 (Table 1C)
RASQRISNYVN

SEQ ID NO: 368 - protein, 11 aa, Artificial Sequence
  VL CDR1 of Ab46, Ab64 and Ab167
RASQSISTYLN

SEQ ID NO: 369 - protein, 12 aa, Artificial Sequence
  VL CDR1 of Ab47 (Table 1C)
RASQRVGTNFLA

SEQ ID NO: 370 - protein, 11 aa, Artificial Sequence
  VL CDR1 of Ab49 and Ab173
RASQGISNSLA

SEQ ID NO: 371 - protein, 11 aa, Artificial Sequence
  VL CDR1 of Ab50 (Table 1C)
RSGQNINKYLN

SEQ ID NO: 372 - protein, 11 aa, Artificial Sequence
  VL CDR1 of Ab51 (Table 1C)
QGDSLRSYYAT

SEQ ID NO: 373 - protein, 11 aa, Artificial Sequence
  VL CDR1 of Ab52 (Table 1C)
RASQGMSNWLA

SEQ ID NO: 374 - protein, 11 aa, Artificial Sequence
  VL CDR1 of Ab54 (Table 1C)
RASQSIDIYLN

SEQ ID NO: 375 - protein, 11 aa, Artificial Sequence
  VL CDR1 of Ab55 (Table 1C)
RASQGISNLLA

SEQ ID NO: 376 - protein, 11 aa, Artificial Sequence
  VL CDR1 of Ab56 (Table 1C)
RASRNVYRYLN

SEQ ID NO: 377 - protein, 11 aa, Artificial Sequence
  VL CDR1 of Ab58 (Table 1C)
RASQSLGSYLA

SEQ ID NO: 378 - protein, 11 aa, Artificial Sequence
  VL CDR1 of Ab59 and Ab66
RASQSISRYLN

SEQ ID NO: 379 - protein, 11 aa, Artificial Sequence
  VL CDR1 of Ab62 (Table 1C)
RASQGISSALA

SEQ ID NO: 380 - protein, 16 aa, Artificial Sequence
  VL CDR1 of Ab63 (Table 1C)
RSSQSLVHSDGNTYLN

SEQ ID NO: 381 - protein, 14 aa, Artificial Sequence
  VL CDR1 of Ab65 (Table 1C)
TGSSSDVGRYNYVS

SEQ ID NO: 382 - protein, 11 aa, Artificial Sequence
  VL CDR1 of Ab67 and Ab91
RASQSVSSNLA

SEQ ID NO: 383 - protein, 8 aa, Artificial Sequence
  VL CDR1 of Ab70 (Table 1C)
TGATRDVS

SEQ ID NO: 384 - protein, 11 aa, Artificial Sequence
  VL CDR1 of Ab71 (Table 1C)
RASQSIGVSLA

SEQ ID NO: 385 - protein, 12 aa, Artificial Sequence
  VL CDR1 of Ab72 (Table 1C)
TLSSGHSSYIIA

SEQ ID NO: 386 - protein, 11 aa, Artificial Sequence
  VL CDR1 of Ab73 (Table 1C)
SGDRLGDKYVS

SEQ ID NO: 387 - protein, 11 aa, Artificial Sequence
  VL CDR1 of Ab74, Ab101 and Ab114
RASQGISNYLA

SEQ ID NO: 388 - protein, 11 aa, Artificial Sequence
  VL CDR1 of Ab76 (Table 1C)
RASRNIKSYLA

SEQ ID NO: 389 - protein, 11 aa, Artificial Sequence
  VL CDR1 of Ab77 (Table 1C)
RASQSIGNNLG

SEQ ID NO: 390 - protein, 11 aa, Artificial Sequence
  VL CDR1 of Ab78 (Table 1C)
QGDSLRRYYAS

SEQ ID NO: 391 - protein, 14 aa, Artificial Sequence
  VL CDR1 of Ab79 (Table 1C)
SGTSSDVGAYNYVS

SEQ ID NO: 392 - protein, 11 aa, Artificial Sequence
  VL CDR1 of Ab81, Ab151 and Ab156
RASQSISSYLN

SEQ ID NO: 393 - protein, 14 aa, Artificial Sequence
  VL CDR1 of Ab82 (Table 1C)
TGASGDVGGYNYVS

SEQ ID NO: 394 - protein, 12 aa, Artificial Sequence
  VL CDR1 of Ab83 (Table 1C)
RASQSVSSNYLA

SEQ ID NO: 395 - protein, 11 aa, Artificial Sequence
  VL CDR1 of Ab84 (Table 1C)
RASQDIRTWLA

SEQ ID NO: 396 - protein, 11 aa, Artificial Sequence
  VL CDR1 of Ab85 (Table 1C)
RASQSIAAWLA

SEQ ID NO: 397 - protein, 12 aa, Artificial Sequence
  VL CDR1 of Ab89 (Table 1C)
RASQSVSNRFLA

SEQ ID NO: 398 - protein, 11 aa, Artificial Sequence
  VL CDR1 of Ab92 (Table 1C)
RASQSVGINLA

SEQ ID NO: 399 - protein, 14 aa, Artificial Sequence
  VL CDR1 of Ab93 (Table 1C)
TGSTSDVGGYTYVS

SEQ ID NO: 400 - protein, 11 aa, Artificial Sequence
  VL CDR1 of Ab95 (Table 1C)
RASQGISRWLA

SEQ ID NO: 401 - protein, 11 aa, Artificial Sequence
  VL CDR1 of Ab96 (Table 1C)
RASQTINRYLN

SEQ ID NO: 402 - protein, 11 aa, Artificial Sequence
  VL CDR1 of Ab97, Ab98 and Ab157
RASQSISSWLA

SEQ ID NO: 403 - protein, 11 aa, Artificial Sequence
  VL CDR1 of Ab99 (Table 1C)
SGDKLGDKFVS

SEQ ID NO: 404 - protein, 12 aa, Artificial Sequence
  VL CDR1 of Ab100 (Table 1C)
RTSQSVSSNSLA

SEQ ID NO: 405 - protein, 16 aa, Artificial Sequence
  VL CDR1 of Ab102 (Table 1C)
RSSQSLLHSNGYTYLE

SEQ ID NO: 406 - protein, 11 aa, Artificial Sequence
  VL CDR1 of Ab103 (Table 1C)
RASQGIRNDLG

SEQ ID NO: 407 - protein, 16 aa, Artificial Sequence
  VL CDR1 of Ab104 (Table 1C)
RSSQSLVYSDGSTYLN

SEQ ID NO: 408 - protein, 16 aa, Artificial Sequence
  VL CDR1 of Ab105 (Table 1C)
HSSHSLLYSDGKTYVN

SEQ ID NO: 409 - protein, 11 aa, Artificial Sequence
  VL CDR1 of Ab106 (Table 1C)
RASQSIDNHLI

SEQ ID NO: 410 - protein, 12 aa, Artificial Sequence
  VL CDR1 of Ab107 (Table 1C)
RASESVSSSHLA

SEQ ID NO: 411 - protein, 14 aa, Artificial Sequence
  VL CDR1 of Ab108 (Table 1C)
TGTSSDVGRYPYVS

SEQ ID NO: 412 - protein, 12 aa, Artificial Sequence
  VL CDR1 of Ab109 (Table 1C)
RASQSLSSSYFA

SEQ ID NO: 413 - protein, 11 aa, Artificial Sequence
  VL CDR1 of Ab110, Ab155 and Ab188
RASQGISSWLA

SEQ ID NO: 414 - protein, 14 aa, Artificial Sequence
  VL CDR1 of Ab111 (Table 1C)
TGTSTDVGGYNFVS

SEQ ID NO: 415 - protein, 12 aa, Artificial Sequence
  VL CDR1 of Ab112 (Table 1C)
RASQRVPSKYLA

SEQ ID NO: 416 - protein, 11 aa, Artificial Sequence
  VL CDR1 of Ab113 (Table 1C)
SGDKLGDKYTS

SEQ ID NO: 417 - protein, 11 aa, Artificial Sequence
  VL CDR1 of Ab115 (Table 1C)
SGNTLSNKFVS

SEQ ID NO: 418 - protein, 11 aa, Artificial Sequence
  VL CDR1 of Ab116 (Table 1C)
RAGQNIGTYLN

SEQ ID NO: 419 - protein, 11 aa, Artificial Sequence
  VL CDR1 of Ab118 (Table 1C)
RGDRLRSYYSS

SEQ ID NO: 420 - protein, 11 aa, Artificial Sequence
  VL CDR1 of Ab119 (Table 1C)
RASQNVRNYLA

SEQ ID NO: 421 - protein, 12 aa, Artificial Sequence
  VL CDR1 of Ab120 (Table 1C)
RASQSLSSSYLA

SEQ ID NO: 422 - protein, 11 aa, Artificial Sequence
  VL CDR1 of Ab122 (Table 1C)
RASQNINTYLN

SEQ ID NO: 423 - protein, 11 aa, Artificial Sequence
  VL CDR1 of Ab123 (Table 1C)
QGDSLRKNYAS

SEQ ID NO: 424 - protein, 12 aa, Artificial Sequence
  VL CDR1 of Ab124 (Table 1C)
RASQSIRSNYLA

SEQ ID NO: 425 - protein, 11 aa, Artificial Sequence
  VL CDR1 of Ab125 (Table 1C)
RASQSISSNLA

SEQ ID NO: 426 - protein, 11 aa, Artificial Sequence
  VL CDR1 of Ab126 (Table 1C)
RASQDISTWLA

SEQ ID NO: 427 - protein, 14 aa, Artificial Sequence
  VL CDR1 of Ab127(Table 1C)
AGTSSDVGSYNYVS

SEQ ID NO: 428 - protein, 11 aa, Artificial Sequence
  VL CDR1 of Ab128 (Table 1C)
RASQSIATYLN

SEQ ID NO: 429 - protein, 11 aa, Artificial Sequence
  VL CDR1 of Ab129 (Table 1C)
SGETLAYKYVS

SEQ ID NO: 430 - protein, 13 aa, Artificial Sequence
  VL CDR1 of Ab131 (Table 1C)
SGSGSNIGTNYVY

SEQ ID NO: 431 - protein, 11 aa, Artificial Sequence
  VL CDR1 of Ab132 (Table 1C)
RASQNINIWLA

SEQ ID NO: 432 - protein, 14 aa, Artificial Sequence
  VL CDR1 of Ab133 (Table 1C)
TGGRRDIGNYNYVS

SEQ ID NO: 433 - protein, 12 aa, Artificial Sequence
  VL CDR1 of Ab135 (Table 1C)
TLSSRYSTYAIA

SEQ ID NO: 434 - protein, 11 aa, Artificial Sequence
  VL CDR1 of Ab136 and Ab160
SGDKLGDKYVS

SEQ ID NO: 435 - protein, 16 aa, Artificial Sequence
  VL CDR1 of Ab137, Ab139 and Ab181
RSSQSLLHSNGYNYLD

SEQ ID NO: 436 - protein, 11 aa, Artificial Sequence
  VL CDR1 of Ab138 (Table 1C)
RASQHISSWLA

SEQ ID NO: 437 - protein, 11 aa, Artificial Sequence
  VL CDR1 of Ab141 (Table 1C)
RASQSVRTYLA

SEQ ID NO: 438 - protein, 12 aa, Artificial Sequence
  VL CDR1 of Ab142 (Table 1C)
RASQSVSSTYLA

SEQ ID NO: 439 - protein, 14 aa, Artificial Sequence
  VL CDR1 of Ab144 (Table 1C)
TGTSSDIGGYNYVS

SEQ ID NO: 440 - protein, 11 aa, Artificial Sequence
  VL CDR1 of Ab148 (Table 1C)
RASQSVSNYLA

SEQ ID NO: 441 - protein, 11 aa, Artificial Sequence
  VL CDR1 of Ab149 (Table 1C)
RASQAISNYLA

SEQ ID NO: 442 - protein, 12 aa, Artificial Sequence
  VL CDR1 of Ab153 (Table 1C)
RASQSVTRNYLA

SEQ ID NO: 443 - protein, 11 aa, Artificial Sequence
  VL CDR1 of Ab154 (Table 1C)
RASQGVSRWLA

SEQ ID NO: 444 - protein, 14 aa, Artificial Sequence
  VL CDR1 of Ab158 (Table 1C)
TGTSSDIGGYNFVS

SEQ ID NO: 445 - protein, 16 aa, Artificial Sequence
  VL CDR1 of Ab159 (Table 1C)
RSSQRLLHSNGYNYLD

SEQ ID NO: 446 - protein, 11 aa, Artificial Sequence
  VL CDR1 of Ab161 (Table 1C)
SGDKLGDKYAS

SEQ ID NO: 447 - protein, 11 aa, Artificial Sequence
  VL CDR1 of Ab162 (Table 1C)
RASQGIRNDLA

SEQ ID NO: 448 - protein, 11 aa, Artificial Sequence
  VL CDR1 of Ab163 (Table 1C)
SGDELGEKYAS

SEQ ID NO: 449 - protein, 17 aa, Artificial Sequence
  VL CDR1 of Ab164 (Table 1C)
KSSQSVLYSSSNKNYLA

SEQ ID NO: 450 - protein, 13 aa, Artificial Sequence
  VL CDR1 of Ab165 (Table 1C)
SGGSSNIGSNFVY

SEQ ID NO: 451 - protein, 11 aa, Artificial Sequence
  VL CDR1 of Ab166 (Table 1C)
RASQSVSSHLA

SEQ ID NO: 452 - protein, 13 aa, Artificial Sequence
  VL CDR1 of Ab168 (Table 1C)
SGSSSNIGGYYVY

SEQ ID NO: 453 - protein, 11 aa, Artificial Sequence
  VL CDR1 of Ab169 (Table 1C)
RSSQNINNFVN

SEQ ID NO: 454 - protein, 11 aa, Artificial Sequence
  VL CDR1 of Ab172 (Table 1C)
RASQSISTSLN

SEQ ID NO: 455 - protein, 11 aa, Artificial Sequence
  VL CDR1 of Ab174 (Table 1C)
RASQGISSSLA

SEQ ID NO: 456 - protein, 12 aa, Artificial Sequence
  VL CDR1 of Ab175 (Table 1C)
RASQSVNSVYLA

SEQ ID NO: 457 - protein, 11 aa, Artificial Sequence
  VL CDR1 of Ab176 (Table 1C)
RASQSVSNYLN

SEQ ID NO: 458 - protein, 13 aa, Artificial Sequence
  VL CDR1 of Ab177 (Table 1C)
SGGNSNIGSHYVY

SEQ ID NO: 459 - protein, 11 aa, Artificial Sequence
  VL CDR1 of Ab178 (Table 1C)
RASQSVSYWLA

SEQ ID NO: 460 - protein, 12 aa, Artificial Sequence
  VL CDR1 of Ab182 (Table 1C)
RASQSVRSSYLA

SEQ ID NO: 461 - protein, 11 aa, Artificial Sequence
  VL CDR1 of Ab184 (Table 1C)
RASQTIGDNLA

SEQ ID NO: 462 - protein, 11 aa, Artificial Sequence
  VL CDR1 of Ab186 (Table 1C)
QASQDISNYLN

SEQ ID NO: 463 - protein, 11 aa, Artificial Sequence
  VL CDR1 of Ab187 (Table 1C)
SGDRLQTKFIS

SEQ ID NO: 464 - protein, 11 aa, Artificial Sequence
  VL CDR1 of Ab190 (Table 1C)
RASQSVGSYLA

SEQ ID NO: 465 - protein, 11 aa, Artificial Sequence
  VL CDR1 of Ab191 (Table 1C)
QGDSLRSYYAS

SEQ ID NO: 466 - protein, 11 aa, Artificial Sequence
  VL CDR1 of Ab192 (Table 1C)
RASQTINRWLA

SEQ ID NO: 467 - protein, 5 aa, Artificial Sequence
  VH CDR1 of Ab24 and Ab146
SYMMV

SEQ ID NO: 468 - protein, 5 aa, Artificial Sequence
  VH CDR1 of Ab25 (Table 3C)
AYQMH

SEQ ID NO: 469 - protein, 5 aa, Artificial Sequence
  VH CDR1 of Ab26 (Table 3C)
YYGMG

SEQ ID NO: 470 - protein, 5 aa, Artificial Sequence
  VH CDR1 of Ab27, Ab101, and Ab110
NYSMH

SEQ ID NO: 471 - protein, 5 aa, Artificial Sequence
  VH CDR1 of Ab28 (Table 3C)
NYRMR

SEQ ID NO: 472 - protein, 5 aa, Artificial Sequence
  VH CDR1 of Ab29 (Table 3C)
NYRMG

SEQ ID NO: 473 - protein, 5 aa, Artificial Sequence
  VH CDR1 of Ab30 and Ab98
RYSMR

SEQ ID NO: 474 - protein, 5 aa, Artificial Sequence
  VH CDR1 of Ab31 (Table 3C)
RYWMG

SEQ ID NO: 475 - protein, 5 aa, Artificial Sequence
  VH CDR1 of Ab32 (Table 3C)
NYRMN

SEQ ID NO: 476 - protein, 5 aa, Artificial Sequence
  VH CDR1 of Ab33 (Table 3C)
WYPMI

SEQ ID NO: 477 - protein, 5 aa, Artificial Sequence
  VH CDR1 of Ab34 (Table 3C)
DYFMF

SEQ ID NO: 478 - protein, 5 aa, Artificial Sequence
  VH CDR1 of Ab35 (Table 3C)
VYPML

SEQ ID NO: 479 - protein, 5 aa, Artificial Sequence
  VH CDR1 of Ab36 (Table 3C)
NYKMI

SEQ ID NO: 480 - protein, 5 aa, Artificial Sequence
  VH CDR1 of Ab38 (Table 3C)
KYPMA

SEQ ID NO: 481 - protein, 5 aa, Artificial Sequence
  VH CDR1 of Ab39 (Table 3C)
FYAMA

SEQ ID NO: 482 - protein, 5 aa, Artificial Sequence
  VH CDR1 of Ab40 (Table 3C)
HYSMG

SEQ ID NO: 483 - protein, 5 aa, Artificial Sequence
  VH CDR1 of Ab41 (Table 3C)
LYPMV

SEQ ID NO: 484 - protein, 5 aa, Artificial Sequence
  VH CDR1 of Ab42 (Table 3C)
NYQMT

SEQ ID NO: 485 - protein, 5 aa, Artificial Sequence
  VH CDR1 of Ab43 and Ab87
NYTMN

SEQ ID NO: 486 - protein, 5 aa, Artificial Sequence
  VH CDR1 of Ab44 and Ab108
NYHMM

SEQ ID NO: 487 - protein, 5 aa, Artificial Sequence
  VH CDR1 of Ab45 (Table 3C)
LYTMT

SEQ ID NO: 488 - protein, 5 aa, Artificial Sequence
  VH CDR1 of Ab46 (Table 3C)
PYSMV

SEQ ID NO: 489 - protein, 5 aa, Artificial Sequence
  VH CDR1 of Ab47 (Table 3C)
RYGMK

SEQ ID NO: 490 - protein, 5 aa, Artificial Sequence
  VH CDR1 of Ab48 (Table 3C)
QYKMY

SEQ ID NO: 491 - protein, 5 aa, Artificial Sequence
  VH CDR1 of Ab49 (Table 3C)
RYSMT

SEQ ID NO: 492 - protein, 5 aa, Artificial Sequence
  VH CDR1 of Ab50 (Table 3C)
NYSMK

SEQ ID NO: 493 - protein, 5 aa, Artificial Sequence
  VH CDR1 of Ab51 (Table 3C)
HYEMM

SEQ ID NO: 494 - protein, 5 aa, Artificial Sequence
  VH CDR1 of Ab52 (Table 3C)
KYMMF

SEQ ID NO: 495 - protein, 5 aa, Artificial Sequence
  VH CDR1 of Ab53 (Table 3C)
NYAMA

SEQ ID NO: 496 - protein, 5 aa, Artificial Sequence
  VH CDR1 of Ab54 and Ab187
KYGMN

SEQ ID NO: 497 - protein, 5 aa, Artificial Sequence
  VH CDR1 of Ab55 (Table 3C)
YYTMF

SEQ ID NO: 498 - protein, 5 aa, Artificial Sequence
  VH CDR1 of Ab56 (Table 3C)
HYHMS

SEQ ID NO: 499 - protein, 5 aa, Artificial Sequence
  VH CDR1 of Ab57 (Table 3C)
KYEMQ

SEQ ID NO: 500 - protein, 5 aa, Artificial Sequence
  VH CDR1 of Ab58 (Table 3C)
NYSMW

SEQ ID NO: 501 - protein, 5 aa, Artificial Sequence
  VH CDR1 of Ab59 (Table 3C)
AYVMS

SEQ ID NO: 502 - protein, 5 aa, Artificial Sequence
  VH CDR1 of Ab60 and Ab180
NYSMN

SEQ ID NO: 503 - protein, 5 aa, Artificial Sequence
  VH CDR1 of Ab62 (Table 3C)
TYWMM

SEQ ID NO: 504 - protein, 5 aa, Artificial Sequence
  VH CDR1 of Ab63 (Table 3C)
RYMMF

SEQ ID NO: 505 - protein, 3 aa, Artificial Sequence
  VH CDR1 of Ab64 (Table 3C)
GMN

SEQ ID NO: 506 - protein, 5 aa, Artificial Sequence
  VH CDR1 of Ab65 (Table 3C)
KYSMT

SEQ ID NO: 507 - protein, 5 aa, Artificial Sequence
  VH CDR1 of Ab66 (Table 3C)
KYHMG

SEQ ID NO: 508 - protein, 5 aa, Artificial Sequence
  VH CDR1 of Ab70 (Table 3C)
WYKMG

SEQ ID NO: 509 - protein, 5 aa, Artificial Sequence
  VH CDR1 of Ab71 (Table 3C)
KYKMV

SEQ ID NO: 510 - protein, 5 aa, Artificial Sequence
  VH CDR1 of Ab72 and Ab106
VYAMG

SEQ ID NO: 511 - protein, 5 aa, Artificial Sequence
  VH CDR1 of Ab73 (Table 3C)
PYPMF

SEQ ID NO: 512 - protein, 5 aa, Artificial Sequence
  VH CDR1 of Ab74 (Table 3C)
SYTMQ

SEQ ID NO: 513 - protein, 5 aa, Artificial Sequence
  VH CDR1 of Ab75 (Table 3C)
NYSMS

SEQ ID NO: 514 - protein, 5 aa, Artificial Sequence
  VH CDR1 of Ab76 (Table 3C)
KYDMY

SEQ ID NO: 515 - protein, 5 aa, Artificial Sequence
  VH CDR1 of Ab77 (Table 3C)
KYVMH

SEQ ID NO: 516 - protein, 5 aa, Artificial Sequence
  VH CDR1 of Ab79 (Table 3C)
YYKMA

SEQ ID NO: 517 - protein, 5 aa, Artificial Sequence
  VH CDR1 of Ab80 (Table 3C)
LYSMN

SEQ ID NO: 518 - protein, 5 aa, Artificial Sequence
  VH CDR1 of Ab81 (Table 3C)
DYFMQ

SEQ ID NO: 519 - protein, 5 aa, Artificial Sequence
  VH CDR1 of Ab82 (Table 3C)
PYMML

SEQ ID NO: 520 - protein, 5 aa, Artificial Sequence
  VH CDR1 of Ab83 (Table 3C)
HYHMV

SEQ ID NO: 521 - protein, 5 aa, Artificial Sequence
  VH CDR1 of Ab84 (Table 3C)
YYQMG

SEQ ID NO: 522 - protein, 5 aa, Artificial Sequence
  VH CDR1 of Ab85 (Table 3C)
YYIMA

SEQ ID NO: 523 - protein, 5 aa, Artificial Sequence
  VH CDR1 of Ab86 (Table 3C)
YYKMF

SEQ ID NO: 524 - protein, 5 aa, Artificial Sequence
  VH CDR1 of Ab88 and Ab185
KYVMY

SEQ ID NO: 525 - protein, 5 aa, Artificial Sequence
  VH CDR1 of Ab89 (Table 3C)
WYTMT

SEQ ID NO: 526 - protein, 5 aa, Artificial Sequence
  VH CDR1 of Ab90 (Table 3C)
KYLMM

SEQ ID NO: 527 - protein, 5 aa, Artificial Sequence
  VH CDR1 of Ab91 (Table 3C)
YYRMW

SEQ ID NO: 528 - protein, 5 aa, Artificial Sequence
  VH CDR1 of Ab92 (Table 3C)
SYQMS

SEQ ID NO: 529 - protein, 5 aa, Artificial Sequence
  VH CDR1 of Ab93 (Table 3C)
PYPMQ

SEQ ID NO: 530 - protein, 5 aa, Artificial Sequence
  VH CDR1 of Ab94 (Table 3C)
KYKMI

SEQ ID NO: 531 - protein, 5 aa, Artificial Sequence
  VH CDR1 of Ab95 (Table 3C)
LYTMM

SEQ ID NO: 532 - protein, 5 aa, Artificial Sequence
  VH CDR1 of Ab96 (Table 3C)
QYNMS

SEQ ID NO: 533 - protein, 5 aa, Artificial Sequence
  VH CDR1 of Ab97 (Table 3C)
PYPMK

SEQ ID NO: 534 - protein, 5 aa, Artificial Sequence
  VH CDR1 of Ab99 (Table 3C)
HYGMG

SEQ ID NO: 535 - protein, 5 aa, Artificial Sequence
  VH CDR1 of Ab100 (Table 3C)
KYSMQ

SEQ ID NO: 536 - protein, 5 aa, Artificial Sequence
  VH CDR1 of Ab102 (Table 3C)
GYMMH

SEQ ID NO: 537 - protein, 5 aa, Artificial Sequence
  VH CDR1 of Ab103 (Table 3C)
AYFMH

SEQ ID NO: 538 - protein, 5 aa, Artificial Sequence
  VH CDR1 of Ab104 (Table 3C)
KYTMG

SEQ ID NO: 539 - protein, 5 aa, Artificial Sequence
  VH CDR1 of Ab105 (Table 3C)
AYPMH

SEQ ID NO: 540 - protein, 5 aa, Artificial Sequence
  VH CDR1 of Ab107 (Table 3C)
HYKMS

SEQ ID NO: 541 - protein, 5 aa, Artificial Sequence
  VH CDR1 of Ab109 (Table 3C)
TYGMQ

SEQ ID NO: 542 - protein, 5 aa, Artificial Sequence
  VH CDR1 of Ab111 (Table 3C)
PYWMI

SEQ ID NO: 543 - protein, 5 aa, Artificial Sequence
  VH CDR1 of Ab112 (Table 3C)
YYNMV

SEQ ID NO: 544 - protein, 5 aa, Artificial Sequence
  VH CDR1 of Ab113 (Table 3C)
DYKMH

SEQ ID NO: 545 - protein, 5 aa, Artificial Sequence
  VH CDR1 of Ab114 (Table 3C)
TYWMH

SEQ ID NO: 546 - protein, 5 aa, Artificial Sequence
  VH CDR1 of Ab115 (Table 3C)
KYRMM

SEQ ID NO: 547 - protein, 5 aa, Artificial Sequence
  VH CDR1 of Ab116 (Table 3C)
FYNMW

SEQ ID NO: 548 - protein, 5 aa, Artificial Sequence
  VH CDR1 of Ab117 (Table 3C)
NYPMG

SEQ ID NO: 549 - protein, 5 aa, Artificial Sequence
  VH CDR1 of Ab118 (Table 3C)
NYAMH

SEQ ID NO: 550 - protein, 5 aa, Artificial Sequence
  VH CDR1 of Ab119 (Table 3C)
HYPMH

SEQ ID NO: 551 - protein, 5 aa, Artificial Sequence
  VH CDR1 of Ab121 (Table 3C)
KYPMH

SEQ ID NO: 552 - protein, 5 aa, Artificial Sequence
  VH CDR1 of Ab122 (Table 3C)
KYRMF

SEQ ID NO: 553 - protein, 5 aa, Artificial Sequence
  VH CDR1 of Ab124 (Table 3C)
WYNMQ

SEQ ID NO: 554 - protein, 5 aa, Artificial Sequence
  VH CDR1 of Ab125 (Table 3C)
NYRMQ

SEQ ID NO: 555 - protein, 5 aa, Artificial Sequence
  VH CDR1 of Ab126 (Table 3C)
WYEMW

SEQ ID NO: 556 - protein, 5 aa, Artificial Sequence
  VH CDR1 of Ab127 (Table 3C)
YYNMM

SEQ ID NO: 557 - protein, 5 aa, Artificial Sequence
  VH CDR1 of Ab128 (Table 3C)
MYRMV

SEQ ID NO: 558 - protein, 5 aa, Artificial Sequence
  VH CDR1 of Ab129 (Table 3C)
KYNMW

SEQ ID NO: 559 - protein, 5 aa, Artificial Sequence
  VH CDR1 of Ab130 (Table 3C)
MYGMA

SEQ ID NO: 560 - protein, 5 aa, Artificial Sequence
  VH CDR1 of Ab131 (Table 3C)
SYGMW

SEQ ID NO: 561 - protein, 5 aa, Artificial Sequence
  VH CDR1 of Ab132 (Table 3C)
EYDMV

SEQ ID NO: 562 - protein, 5 aa, Artificial Sequence
  VH CDR1 of Ab133 (Table 3C)
NYEMH

SEQ ID NO: 563 - protein, 5 aa, Artificial Sequence
  VH CDR1 of Ab134 (Table 3C)
YYTMA

SEQ ID NO: 564 - protein, 5 aa, Artificial Sequence
  VH CDR1 of Ab135 (Table 3C)
AYRMQ

SEQ ID NO: 565 - protein, 5 aa, Artificial Sequence
  VH CDR1 of Ab136 (Table 3C)
KYHMM

SEQ ID NO: 566 - protein, 5 aa, Artificial Sequence
  VH CDR1 of Ab137 (Table 3C)
KYSMR

SEQ ID NO: 567 - protein, 5 aa, Artificial Sequence
  VH CDR1 of Ab138 (Table 3C)
WYNMH

SEQ ID NO: 568 - protein, 5 aa, Artificial Sequence
  VH CDR1 of Ab139 (Table 3C)
WYPMM

SEQ ID NO: 569 - protein, 5 aa, Artificial Sequence
  VH CDR1 of Ab140 (Table 3C)
SYHMQ

SEQ ID NO: 570 - protein, 5 aa, Artificial Sequence
  VH CDR1 of Ab141 (Table 3C)
KYGMQ

SEQ ID NO: 571 - protein, 5 aa, Artificial Sequence
  VH CDR1 of Ab142 (Table 3C)
HYRMA

SEQ ID NO: 572 - protein, 5 aa, Artificial Sequence
  VH CDR1 of Ab143 (Table 3C)
KYSML

SEQ ID NO: 573 - protein, 5 aa, Artificial Sequence
  VH CDR1 of Ab144 (Table 3C)
KYIMT

SEQ ID NO: 574 - protein, 5 aa, Artificial Sequence
  VH CDR1 of Ab145 (Table 3C)
IYRMQ

SEQ ID NO: 575 - protein, 5 aa, Artificial Sequence
  VH CDR1 of Ab147 (Table 3C)
IYKMS

SEQ ID NO: 576 - protein, 5 aa, Artificial Sequence
  VH CDR1 of Ab148 (Table 3C)
AYVMT

SEQ ID NO: 577 - protein, 5 aa, Artificial Sequence
  VH CDR1 of Ab149 (Table 3C)
AYSMH

SEQ ID NO: 578 - protein, 5 aa, Artificial Sequence
  VH CDR1 of Ab151 (Table 3C)
VYGMG

SEQ ID NO: 579 - protein, 5 aa, Artificial Sequence
  VH CDR1 of Ab152 (Table 3C)
NYKMV

SEQ ID NO: 580 - protein, 5 aa, Artificial Sequence
  VH CDR1 of Ab153 (Table 3C)
PYHMT

SEQ ID NO: 581 - protein, 5 aa, Artificial Sequence
  VH CDR1 of Ab154 (Table 3C)
QYHMT

SEQ ID NO: 582 - protein, 5 aa, Artificial Sequence
  VH CDR1 of Ab155 (Table 3C)
SYSMW

SEQ ID NO: 583 - protein, 5 aa, Artificial Sequence
  VH CDR1 of Ab156 (Table 3C)
IYGMH

SEQ ID NO: 584 - protein, 5 aa, Artificial Sequence
  VH CDR1 of Ab157 (Table 3C)
KYPMG

SEQ ID NO: 585 - protein, 5 aa, Artificial Sequence
  VH CDR1 of Ab158 (Table 3C)
LYGMH

SEQ ID NO: 586 - protein, 5 aa, Artificial Sequence
  VH CDR1 of Ab159 (Table 3C)
KYVMI

SEQ ID NO: 587 - protein, 5 aa, Artificial Sequence
  VH CDR1 of Ab160 (Table 3C)
KYMMG

SEQ ID NO: 588 - protein, 5 aa, Artificial Sequence
  VH CDR1 of Ab161 (Table 3C)
KYYMH

SEQ ID NO: 589 - protein, 5 aa, Artificial Sequence
  VH CDR1 of Ab162 (Table 3C)
QYMMS

SEQ ID NO: 590 - protein, 5 aa, Artificial Sequence
  VH CDR1 of Ab163 (Table 3C)
NYGMI

SEQ ID NO: 591 - protein, 5 aa, Artificial Sequence
  VH CDR1 of Ab164 (Table 3C)
HYPMY

SEQ ID NO: 592 - protein, 5 aa, Artificial Sequence
  VH CDR1 of Ab165 (Table 3C)
RYKMV

SEQ ID NO: 593 - protein, 5 aa, Artificial Sequence
  VH CDR1 of Ab166 and Ab171
NYSMG

SEQ ID NO: 594 - protein, 5 aa, Artificial Sequence
  VH CDR1 of Ab167 (Table 3C)
DYSMG

SEQ ID NO: 595 - protein, 5 aa, Artificial Sequence
  VH CDR1 of Ab168 (Table 3C)
SYQMI

SEQ ID NO: 596 - protein, 5 aa, Artificial Sequence
  VH CDR1 of Ab169 (Table 3C)
VYNMR

SEQ ID NO: 597 - protein, 5 aa, Artificial Sequence
  VH CDR1 of Ab170 (Table 3C)
RYKMG

SEQ ID NO: 598 - protein, 5 aa, Artificial Sequence
  VH CDR1 of Ab172 (Table 3C)
QYWMG

SEQ ID NO: 599 - protein, 5 aa, Artificial Sequence
  VH CDR1 of Ab173 (Table 3C)
KYPMI

SEQ ID NO: 600 - protein, 5 aa, Artificial Sequence
  VH CDR1 of Ab174 (Table 3C)
WYPMG

SEQ ID NO: 601 - protein, 5 aa, Artificial Sequence
  VH CDR1 of Ab175 (Table 3C)
RYPMA

SEQ ID NO: 602 - protein, 5 aa, Artificial Sequence
  VH CDR1 of Ab176 (Table 3C)
NYGMF

SEQ ID NO: 603 - protein, 5 aa, Artificial Sequence
  VH CDR1 of Ab177 (Table 3C)
VYWMH

SEQ ID NO: 604 - protein, 5 aa, Artificial Sequence
  VH CDR1 of Ab178 (Table 3C)
KYKMA

SEQ ID NO: 605 - protein, 5 aa, Artificial Sequence
  VH CDR1 of Ab179 (Table 3C)
NYTMF

SEQ ID NO: 606 - protein, 5 aa, Artificial Sequence
  VH CDR1 of Ab181 (Table 3C)
KYWMN

SEQ ID NO: 607 - protein, 5 aa, Artificial Sequence
  VH CDR1 of Ab182 (Table 3C)
FYNMH

SEQ ID NO: 608 - protein, 5 aa, Artificial Sequence
  VH CDR1 of Ab183 (Table 3C)
YYKMV

SEQ ID NO: 609 - protein, 5 aa, Artificial Sequence
  VH CDR1 of Ab184 (Table 3C)
NYRMF

SEQ ID NO: 610 - protein, 5 aa, Artificial Sequence
  VH CDR1 of Ab186 (Table 3C)
KYPMM

SEQ ID NO: 611 - protein, 5 aa, Artificial Sequence
  VH CDR1 of Ab188 (Table 3C)
MYAMH

SEQ ID NO: 612 - protein, 5 aa, Artificial Sequence
  VH CDR1 of Ab189 (Table 3C)
VYTMY

SEQ ID NO: 613 - protein, 5 aa, Artificial Sequence
  VH CDR1 of Ab190 (Table 3C)
NYTMV

SEQ ID NO: 614 - protein, 5 aa, Artificial Sequence
  VH CDR1 of Ab191 (Table 3C)
DYAMA

SEQ ID NO: 615 - protein, 5 aa, Artificial Sequence
  VH CDR1 of Ab192 (Table 3C)
HYSMI

SEQ ID NO: 616 - protein, 7 aa, Artificial Sequence
  VL CDR2 of Ab25 (Table 1C)
KASTLES

SEQ ID NO: 617 - protein, 7 aa, Artificial Sequence
  VL CDR2 of Ab26 (Table 1C)
PASTLQS

SEQ ID NO: 618 - protein, 7 aa, Artificial Sequence
  VL CDR2 of Ab27 (Table 1C)
RASSRAT

SEQ ID NO: 619 - protein, 7 aa, Artificial Sequence
  VL CDR2 of Ab28 (Table 1C)
KASTLEN

SEQ ID NO: 620 - protein, 7 aa, Artificial Sequence
  VL CDR2 of Ab29, Ab60 and Ab180
GASNRAT

SEQ ID NO: 621 - protein, 7 aa, Artificial Sequence
  VL CDR2 of Ab30 (Table 1C)
KASNLEN

SEQ ID NO: 622 - protein, 7 aa, Artificial Sequence
  VL CDR2 of Ab33, Ab118, and 140
GRNNRPS

SEQ ID NO: 623 - protein, 5 aa, Artificial Sequence
  VL CDR2 of Ab34 (Table 1C)
DASTR

SEQ ID NO: 624 - protein, 7 aa, Artificial Sequence
  VL CDR2 of Ab35, Ab71, Ab97 and Ab98
KASSLES

SEQ ID NO: 625 - protein, 7 aa, Artificial Sequence
  VL CDR2 of Ab36 (Table 1C)
GASSRAK

SEQ ID NO: 626 - protein, 7 aa, Artificial Sequence
  VL CDR2 of Ab40 (Table 1C)
RTSTLQS

SEQ ID NO: 627 - protein, 7 aa, Artificial Sequence
  VL CDR2 of Ab42 (Table 1C)
VVSNLQS

SEQ ID NO: 628 - protein, 7 aa, Artificial Sequence
  VL CDR2 of Ab43, Ab55 and Ab125
DASTRAT

SEQ ID NO: 629 - protein, 7 aa, Artificial Sequence
  VL CDR2 of Ab46 and Ab128
AASTLQN

SEQ ID NO: 630 - protein, 7 aa, Artificial Sequence
  VL CDR2 of Ab47 (Table 1C)
GTSTRAA

SEQ ID NO: 631 - protein, 7 aa, Artificial Sequence
  VL CDR2 of Ab48 (Table 1C)
DASKRAT

SEQ ID NO: 632 - protein, 7 aa, Artificial Sequence
  VL CDR2 of Ab49 (Table 1C)
AASRLES

SEQ ID NO: 633 - protein, 7 aa, Artificial Sequence
  VL CDR2 of Ab50 (Table 1C)
AASNVQG

SEQ ID NO: 634 - protein, 7 aa, Artificial Sequence
  VL CDR2 of Ab51 (Table 1C)
GNNNRPS

SEQ ID NO: 635 - protein, 7 aa, Artificial Sequence
  VL CDR2 of Ab52 (Table 1C)
DASSLQS

SEQ ID NO: 636 - protein, 7 aa, Artificial Sequence
  VL CDR2 of Ab56 (Table 1C)
ASSTLQN

SEQ ID NO: 637 - protein, 7 aa, Artificial Sequence
  VL CDR2 of Ab62 (Table 1C)
DASSLES

SEQ ID NO: 638 - protein, 7 aa, Artificial Sequence
  VL CDR2 of Ab63 and Ab104
KVSNRDS

SEQ ID NO: 639 - protein, 7 aa, Artificial Sequence
  VL CDR2 of Ab64 (Table 1C)
GASKLQD

SEQ ID NO: 640 - protein, 7 aa, Artificial Sequence
  VL CDR2 of Ab65 (Table 1C)
DVSERPS

SEQ ID NO: 641 - protein, 7 aa, Artificial Sequence
  VL CDR2 of Ab70 (Table 1C)
EVTSRPS

SEQ ID NO: 642 - protein, 11 aa, Artificial Sequence
  VL CDR2 of Ab72 (Table 1C)
LEGSGSYNKGS

SEQ ID NO: 643 - protein, 7 aa, Artificial Sequence
  VL CDR2 of Ab73 (Table 1C)
EDSKRPS

SEQ ID NO: 644 - protein, 7 aa, Artificial Sequence
  VL CDR2 of Ab74 and Ab101
AASTLQS

SEQ ID NO: 645 - protein, 5 aa, Artificial Sequence
  VL CDR2 of Ab76 (Table 1C)
KASKL

SEQ ID NO: 646 - protein, 7 aa, Artificial Sequence
  VL CDR2 of Ab78 (Table 1C)
GQNTRPS

SEQ ID NO: 647 - protein, 7 aa, Artificial Sequence
  VL CDR2 of Ab79 (Table 1C)
DVNKRPS

SEQ ID NO: 648 - protein, 7 aa, Artificial Sequence
  VL CDR2 of Ab82 (Table 1C)
EGSKRPS

SEQ ID NO: 649 - protein, 7 aa, Artificial Sequence
  VL CDR2 of Ab84 (Table 1C)
AASRLQS

SEQ ID NO: 650 - protein, 7 aa, Artificial Sequence
  VL CDR2 of Ab85 and Ab149
SASTLQS

SEQ ID NO: 651 - protein, 7 aa, Artificial Sequence
  VL CDR2 of Ab89 (Table 1C)
GTSNRAT

SEQ ID NO: 652 - protein, 7 aa, Artificial Sequence
  VL CDR2 of Ab92 (Table 1C)
GASSLQG

SEQ ID NO: 653 - protein, 7 aa, Artificial Sequence
  VL CDR2 of Ab93 (Table 1C)
DVSKRPS

SEQ ID NO: 654 - protein, 7 aa, Artificial Sequence
  VL CDR2 of Ab94 (Table 1C)
GASSRAS

SEQ ID NO: 655 - protein, 7 aa, Artificial Sequence
  VL CDR2 of Ab95 (Table 1C)
AATVLQP

SEQ ID NO: 656 - protein, 7 aa, Artificial Sequence
  VL CDR2 of Ab96 (Table 1C)
DASTLQR

SEQ ID NO: 657 - protein, 7 aa, Artificial Sequence
  VL CDR2 of Ab99 (Table 1C)
QDKKRPA

SEQ ID NO: 658 - protein, 7 aa, Artificial Sequence
  VL CDR2 of Ab102, Ab139, Ab159 and Ab181
LGSNRAS

SEQ ID NO: 659 - protein, 7 aa, Artificial Sequence
  VL CDR2 of Ab103 (Table 1C)
AASSLQR

SEQ ID NO: 660 - protein, 7 aa, Artificial Sequence
  VL CDR2 of Ab105 (Table 1C)
MVSDRDS

SEQ ID NO: 661 - protein, 7 aa, Artificial Sequence
  VL CDR2 of Ab106 (Table 1C)
GASSMQS

SEQ ID NO: 662 - protein, 7 aa, Artificial Sequence
  VL CDR2 of Ab107 (Table 1C)
GSSNRAT

SEQ ID NO: 663 - protein, 7 aa, Artificial Sequence
  VL CDR2 of Ab108 (Table 1C)
EVSKRPS

SEQ ID NO: 664 - protein, 7 aa, Artificial Sequence
  VL CDR2 of Ab109 (Table 1C)
GASNRAA

SEQ ID NO: 665 - protein, 7 aa, Artificial Sequence
  VL CDR2 of Ab111 (Table 1C)
EVTNRPS

SEQ ID NO: 666 - protein, 7 aa, Artificial Sequence
  VL CDR2 of Ab112 (Table 1C)
DASSRAT

SEQ ID NO: 667 - protein, 7 aa, Artificial Sequence
  VL CDR2 of Ab113 (Table 1C)
QDSKRPS

SEQ ID NO: 668 - protein, 7 aa, Artificial Sequence
  VL CDR2 of Ab114 (Table 1C)
ATSTLQS

SEQ ID NO: 669 - protein, 7 aa, Artificial Sequence
  VL CDR2 of Ab115 (Table 1C)
QDTKRPS

SEQ ID NO: 670 - protein, 7 aa, Artificial Sequence
  VL CDR2 of Ab123 (Table 1C)
GQNNRPS

SEQ ID NO: 671 - protein, 7 aa, Artificial Sequence
  VL CDR2 of Ab124 (Table 1C)
DGSSRAN

SEQ ID NO: 672 - protein, 7 aa, Artificial Sequence
  VL CDR2 of Ab127 (Table 1C)
DVTNRPS

SEQ ID NO: 673 - protein, 7 aa, Artificial Sequence
  VL CDR2 of Ab129 (Table 1C)
EDFKRPS

SEQ ID NO: 674 - protein, 7 aa, Artificial Sequence
  VL CDR2 of Ab131, Ab168 and Ab189
RNNQRPS

SEQ ID NO: 675 - protein, 7 aa, Artificial Sequence
  VL CDR2 of Ab132 (Table 1C)
KASILHN

SEQ ID NO: 676 - protein, 7 aa, Artificial Sequence
  VL CDR2 of Ab133 (Table 1C)
DVRKRPS

SEQ ID NO: 677 - protein, 11 aa, Artificial Sequence
  VL CDR2 of Ab135 (Table 1C)
FNTDGSQNKGD

SEQ ID NO: 678 - protein, 7 aa, Artificial Sequence
  VL CDR2 of Ab136 (Table 1C)
QDAKRPS

SEQ ID NO: 679 - protein, 7 aa, Artificial Sequence
  VL CDR2 of Ab137 (Table 1C)
LASNRAS

SEQ ID NO: 680 - protein, 7 aa, Artificial Sequence
  VL CDR2 of Ab138 (Table 1C)
KTSNLES

SEQ ID NO: 681 - protein, 7 aa, Artificial Sequence
  VL CDR2 of Ab144 (Table 1C)
EVNKRPS

SEQ ID NO: 682 - protein, 7 aa, Artificial Sequence
  VL CDR2 of Ab153 (Table 1C)
GTSTRAT

SEQ ID NO: 683 - protein, 7 aa, Artificial Sequence
  VL CDR2 of Ab158 (Table 1C)
QVSDRPS

SEQ ID NO: 684 - protein, 7 aa, Artificial Sequence
  VL CDR2 of Ab160 and Ab187
QDYKRPS

SEQ ID NO: 685 - protein, 7 aa, Artificial Sequence
  VL CDR2 of Ab161 (Table 1C)
QDRKRPS

SEQ ID NO: 686 - protein, 7 aa, Artificial Sequence
  VL CDR2 of Ab162 (Table 1C)
AASGLQS

SEQ ID NO: 687 - protein, 7 aa, Artificial Sequence
  VL CDR2 of Ab163 (Table 1C)
QDNKRPS

SEQ ID NO: 688 - protein, 7 aa, Artificial Sequence
  VL CDR2 of Ab164 (Table 1C)
WASTRES

SEQ ID NO: 689 - protein, 7 aa, Artificial Sequence
  VL CDR2 of Ab165 (Table 1C)
RNYQRPS

SEQ ID NO: 690 - protein, 7 aa, Artificial Sequence
  VL CDR2 of Ab166 (Table 1C)
NAFNRAT

SEQ ID NO: 691 - protein, 7 aa, Artificial Sequence
  VL CDR2 of Ab167 (Table 1C)
AASSLHT

SEQ ID NO: 692 - protein, 7 aa, Artificial Sequence
  VL CDR2 of Ab169 (Table 1C)
AASRLKG

SEQ ID NO: 693 - protein, 7 aa, Artificial Sequence
  VL CDR2 of Ab174 (Table 1C)
GASRLES

SEQ ID NO: 694 - protein, 7 aa, Artificial Sequence
  VL CDR2 of Ab175 (Table 1C)
NASNRAT

SEQ ID NO: 695 - protein, 7 aa, Artificial Sequence
  VL CDR2 of Ab176 (Table 1C)
ATSNLQS

SEQ ID NO: 696 - protein, 7 aa, Artificial Sequence
  VL CDR2 of Ab177 (Table 1C)
RDNQRPS

SEQ ID NO: 697 - protein, 7 aa, Artificial Sequence
  VL CDR2 of Ab178 (Table 1C)
RASRLES

SEQ ID NO: 698 - protein, 7 aa, Artificial Sequence
  VL CDR2 of Ab184 (Table 1C)
GASMRAT

SEQ ID NO: 699 - protein, 7 aa, Artificial Sequence
  VL CDR2 of Ab186 (Table 1C)
DASNLET

SEQ ID NO: 700 - protein, 7 aa, Artificial Sequence
  VL CDR2 of Ab190 (Table 1C)
DTSNRAT

SEQ ID NO: 701 - protein, 7 aa, Artificial Sequence
  VL CDR2 of Ab191 (Table 1C)
GKNNRPS

SEQ ID NO: 702 - protein, 7 aa, Artificial Sequence
  VL CDR2 of Ab192 (Table 1C)
ATSTLEN

SEQ ID NO: 703 - protein, 17 aa, Artificial Sequence
  VH CDR2 of Ab25 (Table 3C)
SIVPSGGSTNYADSVKG

SEQ ID NO: 704 - protein, 17 aa, Artificial Sequence
  VH CDR2 of Ab26 (Table 3C)
RISSSGGGTRYADSVKG

SEQ ID NO: 705 - protein, 17 aa, Artificial Sequence
  VH CDR2 of Ab27 and Ab101
YIYPSGGGTIYADSVKG

SEQ ID NO: 706 - protein, 17 aa, Artificial Sequence
  VH CDR2 of Ab28 (Table 3C)
GIYPSGGDTQYADSVKG

SEQ ID NO: 707 - protein, 17 aa, Artificial Sequence
  VH CDR2 of Ab29 (Table 3C)
SIVPSGGSTQYADSVKG

SEQ ID NO: 708 - protein, 17 aa, Artificial Sequence
  VH CDR2 of Ab30 (Table 3C)
SISPSGGYTEYADSVKG

SEQ ID NO: 709 - protein, 17 aa, Artificial Sequence
  VH CDR2 of Ab31 (Table 3C)
SISSSGGYTNYADSVKG

SEQ ID NO: 710 - protein, 16 aa, Artificial Sequence
  VH CDR2 of Ab32 (Table 3C)
YIWPSGGGTYADSVKG

SEQ ID NO: 711 - protein, 17 aa, Artificial Sequence
  VH CDR2 of Ab33 (Table 3C)
WISPSGGFTNYADSVKG

SEQ ID NO: 712 - protein, 17 aa, Artificial Sequence
  VH CDR2 of Ab34 (Table 3C)
VISPSGGWTEYADSVKG

SEQ ID NO: 713 - protein, 17 aa, Artificial Sequence
  VH CDR2 of Ab35 (Table 3C)
SISSSGGDTAYADSVKG

SEQ ID NO: 714 - protein, 17 aa, Artificial Sequence
  VH CDR2 of Ab36 (Table 3C)
RIYPSGGNTGYADSVKG

SEQ ID NO: 715 - protein, 17 aa, Artificial Sequence
  VH CDR2 of Ab38 (Table 3C)
VIRSSGGFTTYADSVKG

SEQ ID NO: 716 - protein, 17 aa, Artificial Sequence
  VH CDR2 of Ab39 (Table 3C)
RISSSGGNTQYADSVKG

SEQ ID NO: 717 - protein, 17 aa, Artificial Sequence
  VH CDR2 of Ab40 (Table 3C)
SISPSGGATVYADSVKG

SEQ ID NO: 718 - protein, 17 aa, Artificial Sequence
  VH CDR2 of Ab41 (Table 3C)
VISSSGGHTRYADSVKG

SEQ ID NO: 719 - protein, 17 aa, Artificial Sequence
  VH CDR2 of Ab42 (Table 3C)
SIWSSGGKTEYADSVKG

SEQ ID NO: 720 - protein, 17 aa, Artificial Sequence
  VH CDR2 of Ab43 (Table 3C)
SIVPSGGNTTYADSVKG

SEQ ID NO: 721 - protein, 17 aa, Artificial Sequence
  VH CDR2 of Ab44 (Table 3C)
VISPSGGFTAYADSVKG

SEQ ID NO: 722 - protein, 17 aa, Artificial Sequence
  VH CDR2 of Ab45 (Table 3C)
SISPSGGYTFYADSVKG

SEQ ID NO: 723 - protein, 17 aa, Artificial Sequence
  VH CDR2 of Ab46 (Table 3C)
SIRPSGGNTRYADSVKG

SEQ ID NO: 724 - protein, 17 aa, Artificial Sequence
  VH CDR2 of Ab47 (Table 3C)
SISPSGGLTIYADSVKG

SEQ ID NO: 725 - protein, 17 aa, Artificial Sequence
  VH CDR2 of Ab48 (Table 3C)
VISSSGGMTEYADSVKG

SEQ ID NO: 726 - protein, 17 aa, Artificial Sequence
  VH CDR2 of Ab49 (Table 3C)
SIRSSGGITQYADSVKG

SEQ ID NO: 727 - protein, 17 aa, Artificial Sequence
  VH CDR2 of Ab50 (Table 3C)
SIYPSGGGTMYADSVKG

SEQ ID NO: 728 - protein, 17 aa, Artificial Sequence
  VH CDR2 of Ab51 (Table 3C)
RIVSSGGKTHYADSVKG

SEQ ID NO: 729 - protein, 17 aa, Artificial Sequence
  VH CDR2 of Ab52 (Table 3C)
GIYSSGGWTGYADSVKG

SEQ ID NO: 730 - protein, 17 aa, Artificial Sequence
  VH CDR2 of Ab53 (Table 3C)
RIWPSGGGTLYADSVKG

SEQ ID NO: 731 - protein, 17 aa, Artificial Sequence
  VH CDR2 of Ab54 (Table 3C)
GIYPSGGETAYADSVKG

SEQ ID NO: 732 - protein, 17 aa, Artificial Sequence
  VH CDR2 of Ab55 (Table 3C)
RIVPSGGSTNYADSVKG

SEQ ID NO: 733 - protein, 17 aa, Artificial Sequence
  VH CDR2 of Ab56 (Table 3C)
RIYSSGGSTMYADSVKG

SEQ ID NO: 734 - protein, 17 aa, Artificial Sequence
  VH CDR2 of Ab57 (Table 3C)
GISPSGGITHYADSVKG

SEQ ID NO: 735 - protein, 17 aa, Artificial Sequence
  VH CDR2 of Ab58 and Ab180
RIYPSGGNTFYADSVKG

SEQ ID NO: 736 - protein, 17 aa, Artificial Sequence
  VH CDR2 of Ab59 (Table 3C)
SIGPSGGLTIYADSVKG

SEQ ID NO: 737 - protein, 17 aa, Artificial Sequence
  VH CDR2 of Ab60 (Table 3C)
GIWPSGGNTFYADSVKG

SEQ ID NO: 738 - protein, 17 aa, Artificial Sequence
  VH CDR2 of Ab62 (Table 3C)
SIVPSGGFTSYADSVKG

SEQ ID NO: 739 - protein, 17 aa, Artificial Sequence
  VH CDR2 of Ab63 (Table 3C)
VISPSGGWTRYADSVKG

SEQ ID NO: 740 - protein, 17 aa, Artificial Sequence
  VH CDR2 of Ab64 (Table 3C)
YISSSGGATIYADSVKG

SEQ ID NO: 741 - protein, 17 aa, Artificial Sequence
  VH CDR2 of Ab65 (Table 3C)
RISSSGGYTPYADSVKG

SEQ ID NO: 742 - protein, 17 aa, Artificial Sequence
  VH CDR2 of Ab66 (Table 3C)
WIYPSGGNTTYADSVKG

SEQ ID NO: 743 - protein, 17 aa, Artificial Sequence
  VH CDR2 of Ab70 (Table 3C)
SIYSSGGSTRYADSVKG

SEQ ID NO: 744 - protein, 17 aa, Artificial Sequence
  VH CDR2 of Ab71 (Table 3C)
VISPSGGITHYADSVKG

SEQ ID NO: 745 - protein, 17 aa, Artificial Sequence
  VH CDR2 of Ab72 (Table 3C)
SISPSGGVTKYADSVKG

SEQ ID NO: 746 - protein, 17 aa, Artificial Sequence
  VH CDR2 of Ab73 (Table 3C)
SISPSGGATHYADSVKG

SEQ ID NO: 747 - protein, 17 aa, Artificial Sequence
  VH CDR2 of Ab74 (Table 3C)
GIYPSGGGTMYADSVKG

SEQ ID NO: 748 - protein, 17 aa, Artificial Sequence
  VH CDR2 of Ab75 (Table 3C)
YIYPSGGETMYADSVKG

SEQ ID NO: 749 - protein, 17 aa, Artificial Sequence
  VH CDR2 of Ab76 (Table 3C)
YISSSGGSTWYADSVKG

SEQ ID NO: 750 - protein, 17 aa, Artificial Sequence
  VH CDR2 of Ab77 (Table 3C)
RIGPSGGKTHYADSVKG

SEQ ID NO: 751 - protein, 17 aa, Artificial Sequence
  VH CDR2 of Ab78 (Table 3C)
RIYPSGGGTAYADSVKG

SEQ ID NO: 752 - protein, 17 aa, Artificial Sequence
  VH CDR2 of Ab79 (Table 3C)
VIYPSGGYTKYADSVKG

SEQ ID NO: 753 - protein, 17 aa, Artificial Sequence
  VH CDR2 of Ab80 (Table 3C)
RISSSGGETQYADSVKG

SEQ ID NO: 754 - protein, 17 aa, Artificial Sequence
  VH CDR2 of Ab81 (Table 3C)
GIGPSGGPTQYADSVKG

SEQ ID NO: 755 - protein, 17 aa, Artificial Sequence
  VH CDR2 of Ab82 (Table 3C)
WIVPSGGDTAYADSVKG

SEQ ID NO: 756 - protein, 17 aa, Artificial Sequence
  VH CDR2 of Ab83 (Table 3C)
VISPSGGKTRYADSVKG

SEQ ID NO: 757 - protein, 17 aa, Artificial Sequence
  VH CDR2 of Ab84 (Table 3C)
SISSSGGSTIYADSVKG

SEQ ID NO: 758 - protein, 17 aa, Artificial Sequence
  VH CDR2 of Ab85 (Table 3C)
SIGSSGGMTAYADSVKG

SEQ ID NO: 759 - protein, 17 aa, Artificial Sequence
  VH CDR2 of Ab86 (Table 3C)
GIYPSGGATEYADSVKG

SEQ ID NO: 760 - protein, 17 aa, Artificial Sequence
  VH CDR2 of Ab87 (Table 3C)
RIWPSGGSNFYADSVKG

SEQ ID NO: 761 - protein, 17 aa, Artificial Sequence
  VH CDR2 of Ab88 (Table 3C)
GIVPSGGLTQYADSVKG

SEQ ID NO: 762 - protein, 17 aa, Artificial Sequence
  VH CDR2 of Ab89 (Table 3C)
SIWPSGGGTDYADSVKG

SEQ ID NO: 763 - protein, 17 aa, Artificial Sequence
  VH CDR2 of Ab90 (Table 3C)
YISSSGGHTYYADSVKG

SEQ ID NO: 764 - protein, 17 aa, Artificial Sequence
  VH CDR2 of Ab91 (Table 3C)
RIGPSGGTTNYADSVKG

SEQ ID NO: 765 - protein, 17 aa, Artificial Sequence
  VH CDR2 of Ab92 (Table 3C)
YIWPSGGATSYADSVKG

SEQ ID NO: 766 - protein, 17 aa, Artificial Sequence
  VH CDR2 of Ab93 (Table 3C)
SISSSGGFTSYADSVKG

SEQ ID NO: 767 - protein, 17 aa, Artificial Sequence
  VH CDR2 of Ab94 (Table 3C)
RISSSGGRTKYADSVKG

SEQ ID NO: 768 - protein, 17 aa, Artificial Sequence
  VH CDR2 of Ab95 (Table 3C)
YIGSSGGNTNYADSVKG

SEQ ID NO: 769 - protein, 17 aa, Artificial Sequence
  VH CDR2 of Ab96 (Table 3C)
SIYPSGGVTGYADSVKG

SEQ ID NO: 770 - protein, 17 aa, Artificial Sequence
  VH CDR2 of Ab97 (Table 3C)
SIWSSGGPTQYADSVKG

SEQ ID NO: 771 - protein, 17 aa, Artificial Sequence
  VH CDR2 of Ab99 (Table 3C)
YISSSGGQTKYADSVKG

SEQ ID NO: 772 - protein, 17 aa, Artificial Sequence
  VH CDR2 of Ab100 (Table 3C)
GIGSSGGLTSYADSVKG

SEQ ID NO: 773 - protein, 17 aa, Artificial Sequence
  VH CDR2 of Ab102 (Table 3C)
GIGSSGGWTSYADSVKG

SEQ ID NO: 774 - protein, 17 aa, Artificial Sequence
  VH CDR2 of Ab103 (Table 3C)
SISSSGGSTLYADSVKG

SEQ ID NO: 775 - protein, 17 aa, Artificial Sequence
  VH CDR2 of Ab104 (Table 3C)
YIGSSGGKTTYADSVKG

SEQ ID NO: 776 - protein, 17 aa, Artificial Sequence
  VH CDR2 of Ab105 (Table 3C)
SIWPSGGFTIYADSVKG

SEQ ID NO: 777 - protein, 17 aa, Artificial Sequence
  VH CDR2 of Ab106 (Table 3C)
GIGPSGGTTHYADSVKG

SEQ ID NO: 778 - protein, 17 aa, Artificial Sequence
  VH CDR2 of Ab107 (Table 3C)
GIYPSGGPTGYADSVKG

SEQ ID NO: 779 - protein, 16 aa, Artificial Sequence
  VH CDR2 of Ab108 (Table 3C)
SISSGGKTYYADSVKG

SEQ ID NO: 780 - protein, 17 aa, Artificial Sequence
  VH CDR2 of Ab109 (Table 3C)
YISSSGGWTQYANSVKG

SEQ ID NO: 781 - protein, 17 aa, Artificial Sequence
  VH CDR2 of Ab110 (Table 3C)
RIYPSGGITPYADSVKG

SEQ ID NO: 782 - protein, 17 aa, Artificial Sequence
  VH CDR2 of Ab111 (Table 3C)
RISPSGGDTKYADSVKG

SEQ ID NO: 783 - protein, 17 aa, Artificial Sequence
  VH CDR2 of Ab112 (Table 3C)
SIYPSGGLTQYADSVKG

SEQ ID NO: 784 - protein, 17 aa, Artificial Sequence
  VH CDR2 of Ab113 (Table 3C)
GIWPSGGATSYADSVKG

SEQ ID NO: 785 - protein, 16 aa, Artificial Sequence
  VH CDR2 of Ab114 (Table 3C)
SIYSSGNTVYADSVKG

SEQ ID NO: 786 - protein, 17 aa, Artificial Sequence
  VH CDR2 of Ab115 (Table 3C)
YISSSGGVTEYADSVKG

SEQ ID NO: 787 - protein, 17 aa, Artificial Sequence
  VH CDR2 of Ab116 (Table 3C)
RISSSGGFTYYADSVKG

SEQ ID NO: 788 - protein, 17 aa, Artificial Sequence
  VH CDR2 of Ab117 (Table 3C)
YIVPSGGSTAYADPVKG

SEQ ID NO: 789 - protein, 17 aa, Artificial Sequence
  VH CDR2 of Ab118 (Table 3C)
YIVPSGGSTHYADSVKG

SEQ ID NO: 790 - protein, 17 aa, Artificial Sequence
  VH CDR2 of Ab119 (Table 3C)
SIVSSGGFTQYADSVKG

SEQ ID NO: 791 - protein, 17 aa, Artificial Sequence
  VH CDR2 of Ab120 (Table 3C)
VISPSGGYTAYADSVKG

SEQ ID NO: 792 - protein, 17 aa, Artificial Sequence
  VH CDR2 of Ab121 (Table 3C)
SIVSSGGTTKYADSVKG

SEQ ID NO: 793 - protein, 17 aa, Artificial Sequence
  VH CDR2 of Ab122 (Table 3C)
YISPSGGYTAYADSVKG

SEQ ID NO: 794 - protein, 17 aa, Artificial Sequence
  VH CDR2 of Ab124 (Table 3C)
SIWPSGGATNYADSVKG

SEQ ID NO: 795 - protein, 17 aa, Artificial Sequence
  VH CDR2 of Ab125 (Table 3C)
WIYPSGGGTWYADSVKG

SEQ ID NO: 796 - protein, 17 aa, Artificial Sequence
  VH CDR2 of Ab126 (Table 3C)
YIGSSGGDTWYADSVKG

SEQ ID NO: 797 - protein, 17 aa, Artificial Sequence
  VH CDR2 of Ab127 (Table 3C)
SISPSGGHTTYADSVKG

SEQ ID NO: 798 - protein, 17 aa, Artificial Sequence
  VH CDR2 of Ab128 (Table 3C)
SIVSSGGFTKYADSVKG

SEQ ID NO: 799 - protein, 17 aa, Artificial Sequence
  VH CDR2 of Ab129 (Table 3C)
GIVPSGGYTLYADSVKG

SEQ ID NO: 800 - protein, 17 aa, Artificial Sequence
  VH CDR2 of Ab130 (Table 3C)
YISSSGGTTIYADSVKG

SEQ ID NO: 801 - protein, 17 aa, Artificial Sequence
  VH CDR2 of Ab131 (Table 3C)
SIGPSGGYTRYADSVKG

SEQ ID NO: 802 - protein, 17 aa, Artificial Sequence
  VH CDR2 of Ab132 (Table 3C)
YISPSGGKTSYADSVKG

SEQ ID NO: 803 - protein, 17 aa, Artificial Sequence
  VH CDR2 of Ab133 (Table 3C)
GISPSGGWTQYADSVKG

SEQ ID NO: 804 - protein, 17 aa, Artificial Sequence
  VH CDR2 of Ab134 (Table 3C)
SIRPSGGSTTYADSVKG

SEQ ID NO: 805 - protein, 17 aa, Artificial Sequence
  VH CDR2 of Ab135 (Table 3C)
WIYPSGGATLYADSVKG

SEQ ID NO: 806 - protein, 17 aa, Artificial Sequence
  VH CDR2 of Ab136 (Table 3C)
GIVSSGGSTAYADSVKG

SEQ ID NO: 807 - protein, 17 aa, Artificial Sequence
  VH CDR2 of Ab137 (Table 3C)
SISSSGGTTWYADSVKG

SEQ ID NO: 808 - protein, 17 aa, Artificial Sequence
  VH CDR2 of Ab138 (Table 3C)
SIWSSGGGTQYADSVKG

SEQ ID NO: 809 - protein, 17 aa, Artificial Sequence
  VH CDR2 of Ab139 (Table 3C)
RIGSSGGRTDYADSVKG

SEQ ID NO: 810 - protein, 17 aa, Artificial Sequence
  VH CDR2 of Ab140 (Table 3C)
RISPSGGSTWYADSVKG

SEQ ID NO: 811 - protein, 17 aa, Artificial Sequence
  VH CDR2 of Ab141 (Table 3C)
YIGSSGGLTRYADSVKG

SEQ ID NO: 812 - protein, 17 aa, Artificial Sequence
  VH CDR2 of Ab142 (Table 3C)
GIYPSGGMTSYADSVKG

SEQ ID NO: 813 - protein, 17 aa, Artificial Sequence
  VH CDR2 of Ab143 (Table 3C)
SIGSSGGVTKYADSVKG

SEQ ID NO: 814 - protein, 17 aa, Artificial Sequence
  VH CDR2 of Ab144 (Table 3C)
SISPSGGYTAYADSVKG

SEQ ID NO: 815 - protein, 17 aa, Artificial Sequence
  VH CDR2 of Ab145 (Table 3C)
SIVPSGGSTGYADSVKG

SEQ ID NO: 816 - protein, 17 aa, Artificial Sequence
  VH CDR2 of Ab147 (Table 3C)
SIWPSGGNTTYADSVKG

SEQ ID NO: 817 - protein, 17 aa, Artificial Sequence
  VH CDR2 of Ab148 (Table 3C)
SIWSSGGSTGYADSVKG

SEQ ID NO: 818 - protein, 17 aa, Artificial Sequence
  VH CDR2 of Ab149 (Table 3C)
RIVPSGGLTNYADSVKG

SEQ ID NO: 819 - protein, 17 aa, Artificial Sequence
  VH CDR2 of Ab151 (Table 3C)
YIYPSGGLTRYADSVKG

SEQ ID NO: 820 - protein, 17 aa, Artificial Sequence
  VH CDR2 of Ab152 (Table 3C)
SIYPSGGGTRYADSVKG

SEQ ID NO: 821 - protein, 17 aa, Artificial Sequence
  VH CDR2 of Ab153 (Table 3C)
SIGPSGGNTLYADSVKG

SEQ ID NO: 822 - protein, 17 aa, Artificial Sequence
  VH CDR2 of Ab154 (Table 3C)
SIYSSGGLTNYADSVKG

SEQ ID NO: 823 - protein, 17 aa, Artificial Sequence
  VH CDR2 of Ab155 (Table 3C)
VISPSGGFTRYADSVKG

SEQ ID NO: 824 - protein, 17 aa, Artificial Sequence
  VH CDR2 of Ab156 (Table 3C)
SIVPSGGFTPYADSVKG

SEQ ID NO: 825 - protein, 17 aa, Artificial Sequence
  VH CDR2 of Ab157 (Table 3C)
SIVSSGGSTHYADSVKG

SEQ ID NO: 826 - protein, 17 aa, Artificial Sequence
  VH CDR2 of Ab158 (Table 3C)
SIWSSGGYTTYADSVKG

SEQ ID NO: 827 - protein, 17 aa, Artificial Sequence
  VH CDR2 of Ab159 (Table 3C)
YISSSGGNTRYADSVKG

SEQ ID NO: 828 - protein, 17 aa, Artificial Sequence
  VH CDR2 of Ab160 (Table 3C)
YIGPSGGMTGYADSVKG

SEQ ID NO: 829 - protein, 17 aa, Artificial Sequence
  VH CDR2 of Ab161 (Table 3C)
SISSSGGATFYADSVKG

SEQ ID NO: 830 - protein, 17 aa, Artificial Sequence
  VH CDR2 of Ab162 (Table 3C)
SISPSGGHTYYADSVKG

SEQ ID NO: 831 - protein, 17 aa, Artificial Sequence
  VH CDR2 of Ab163 (Table 3C)
YIYPSGGGTMYADSVKG

SEQ ID NO: 832 - protein, 17 aa, Artificial Sequence
  VH CDR2 of Ab164 (Table 3C)
VISPSGGYTLYADSVKG

SEQ ID NO: 833 - protein, 17 aa, Artificial Sequence
  VH CDR2 of Ab165 (Table 3C)
GIRPSGGLTTYADSVKG

SEQ ID NO: 834 - protein, 17 aa, Artificial Sequence
  VH CDR2 of Ab166 and Ab171
SIYPSGGSTAYADSVKG

SEQ ID NO: 835 - protein, 17 aa, Artificial Sequence
  VH CDR2 of Ab167 (Table 3C)
RISSSGGWTGYADSVKG

SEQ ID NO: 836 - protein, 17 aa, Artificial Sequence
  VH CDR2 of Ab168 (Table 3C)
YIGSSGGTTSYADSVKG

SEQ ID NO: 837 - protein, 17 aa, Artificial Sequence
  VH CDR2 of Ab169 (Table 3C)
SISPSGGRTYYADSVKG

SEQ ID NO: 838 - protein, 17 aa, Artificial Sequence
  VH CDR2 of Ab170 (Table 3C)
SIRSSGGKTLYADSVKG

SEQ ID NO: 839 - protein, 17 aa, Artificial Sequence
  VH CDR2 of Ab172 (Table 3C)
SIRSSGGYTLYADSVKG

SEQ ID NO: 840 - protein, 17 aa, Artificial Sequence
  VH CDR2 of Ab173 (Table 3C)
GIYSSGGMTHYADSVKG

SEQ ID NO: 841 - protein, 17 aa, Artificial Sequence
  VH CDR2 of Ab174 (Table 3C)
WIGSSGGVTRYADSVKG

SEQ ID NO: 842 - protein, 17 aa, Artificial Sequence
  VH CDR2 of Ab175 (Table 3C)
YIYPSGGHTDYADSVKG

SEQ ID NO: 843 - protein, 17 aa, Artificial Sequence
  VH CDR2 of Ab176 (Table 3C)
GIRSSGGQTWYADSVKG

SEQ ID NO: 844 - protein, 17 aa, Artificial Sequence
  VH CDR2 of Ab177 (Table 3C)
SIRPSGGFTFYADSVKG

SEQ ID NO: 845 - protein, 17 aa, Artificial Sequence
  VH CDR2 of Ab178 (Table 3C)
YIGSSGGYTQYADSVKG

SEQ ID NO: 846 - protein, 17 aa, Artificial Sequence
  VH CDR2 of Ab179 (Table 3C)
GIVPSGGSTIYADSVKG

SEQ ID NO: 847 - protein, 17 aa, Artificial Sequence
  VH CDR2 of Ab181 (Table 3C)
VISPSGGSTKYADSVKG

SEQ ID NO: 848 - protein, 17 aa, Artificial Sequence
  VH CDR2 of Ab182 (Table 3C)
GIVPSGGMTSYADSVKG

SEQ ID NO: 849 - protein, 17 aa, Artificial Sequence
  VH CDR2 of Ab183 (Table 3C)
GIYPSGGLTQYADSVKG

SEQ ID NO: 850 - protein, 17 aa, Artificial Sequence
  VH CDR2 of Ab184 (Table 3C)
SIVPSGGSTVYADSVKG

SEQ ID NO: 851 - protein, 17 aa, Artificial Sequence
  VH CDR2 of Ab185 (Table 3C)
GIVPSGGATMYADSVKG

SEQ ID NO: 852 - protein, 17 aa, Artificial Sequence
  VH CDR2 of Ab186 (Table 3C)
SIWSSGGQTYYADSVKG

SEQ ID NO: 853 - protein, 17 aa, Artificial Sequence
  VH CDR2 of Ab187 (Table 3C)
SISSSGGQTEYADSVKG

SEQ ID NO: 854 - protein, 17 aa, Artificial Sequence
  VH CDR2 of Ab188 (Table 3C)
SIRPSGGATNYADSVKG

SEQ ID NO: 855 - protein, 17 aa, Artificial Sequence
  VH CDR2 of Ab189 (Table 3C)
YIGSSGGTTIYADSVKG

SEQ ID NO: 856 - protein, 17 aa, Artificial Sequence
  VH CDR2 of Ab190 (Table 3C)
YIYPSGGGTSYADSVKG

SEQ ID NO: 857 - protein, 17 aa, Artificial Sequence
  VH CDR2 of Ab191 (Table 3C)
WISPSGGWTNYADSVKG

SEQ ID NO: 858 - protein, 17 aa, Artificial Sequence
  VH CDR2 of Ab192 (Table 3C)
VIGPSGGFTRYADSVKG

SEQ ID NO: 859 - protein, 9 aa, Artificial Sequence
  VL CDR3 of Ab24, Ab46 and Ab128
QQSRSTLYT

SEQ ID NO: 860 - protein, 9 aa, Artificial Sequence
  VL CDR3 of Ab25 (Table 1C)
QQSNSFPLT

SEQ ID NO: 861 - protein, 9 aa, Artificial Sequence
  VL CDR3 of Ab26 (Table 1C)
QQSYITLFT

SEQ ID NO: 862 - protein, 10 aa, Artificial Sequence
  VL CDR3 of Ab27 (Table 1C)
QNYGYSPPYT

SEQ ID NO: 863 - protein, 10 aa, Artificial Sequence
  VL CDR3 of Ab28 (Table 1C)
QQYKFYSSYT

SEQ ID NO: 864 - protein, 9 aa, Artificial Sequence
  VL CDR3 of Ab29 (Table 1C)
QQRTNWLIT

SEQ ID NO: 865 - protein, 9 aa, Artificial Sequence
  VL CDR3 of Ab30 (Table 1C)
QQYNTYSWT

SEQ ID NO: 866 - protein, 8 aa, Artificial Sequence
  VL CDR3 of Ab32 (Table 1C)
QQRSNWIT

SEQ ID NO: 867 - protein, 10 aa, Artificial Sequence
  VL CDR3 of Ab33, Ab118 and Ab140
SSRDGSGNFL

SEQ ID NO: 868 - protein, 9 aa, Artificial Sequence
  VL CDR3 of Ab34 (Table 1C)
QQRSTWPRT

SEQ ID NO: 869 - protein, 9 aa, Artificial Sequence
  VL CDR3 of Ab35 (Table 1C)
QQYNSYSQT

SEQ ID NO: 870 - protein, 10 aa, Artificial Sequence
  VL CDR3 of Ab36 (Table 1C)
QHYGRSGPWT

SEQ ID NO: 871 - protein, 9 aa, Artificial Sequence
  VL CDR3 of Ab39 (Table 1C)
LQSHSTPLT

SEQ ID NO: 872 - protein, 9 aa, Artificial Sequence
  VL CDR3 of Ab40 (Table 1C)
QQYDKYPIT

SEQ ID NO: 873 - protein, 10 aa, Artificial Sequence
  VL CDR3 of Ab41 (Table 1C)
QQRSNWPPFT

SEQ ID NO: 874 - protein, 9 aa, Artificial Sequence
  VL CDR3 of Ab42 (Table 1C)
QQSYSTPQT

SEQ ID NO: 875 - protein, 11 aa, Artificial Sequence
  VL CDR3 of Ab43 (Table 1C)
QQYNNWPPMFT

SEQ ID NO: 876 - protein, 9 aa, Artificial Sequence
  VL CDR3 of Ab44 (Table 1C)
QQSYSLPFT

SEQ ID NO: 877 - protein, 10 aa, Artificial Sequence
  VL CDR3 of Ab45 (Table 1C)
QQRSNWPSIT

SEQ ID NO: 878 - protein, 9 aa, Artificial Sequence
  VL CDR3 of Ab47 (Table 1C)
QQYEASPYT

SEQ ID NO: 879 - protein, 9 aa, Artificial Sequence
  VL CDR3 of Ab48, Ab80, Ab88, Ab117 and Ab185
QQRSNWPLT

SEQ ID NO: 880 - protein, 9 aa, Artificial Sequence
  VL CDR3 of Ab49 (Table 1C)
QQYYSTPFT

SEQ ID NO: 881 - protein, 9 aa, Artificial Sequence
  VL CDR3 of Ab50 (Table 1C)
QESFRSSRT

SEQ ID NO: 882 - protein, 11 aa, Artificial Sequence
  VL CDR3 of Ab51 (Table 1C)
KSRDSSRNHVV

SEQ ID NO: 883 - protein, 9 aa, Artificial Sequence
  VL CDR3 of Ab52 (Table 1C)
LQVHSFPLT

SEQ ID NO: 884 - protein, 10 aa, Artificial Sequence
  VL CDR3 of Ab53 (Table 1C)
QQRSNWPPIT

SEQ ID NO: 885 - protein, 8 aa, Artificial Sequence
  VL CDR3 of Ab55 (Table 1C)
HQRSYRGT

SEQ ID NO: 886 - protein, 9 aa, Artificial Sequence
  VL CDR3 of Ab56 (Table 1C)
QQSYQTPFT

SEQ ID NO: 887 - protein, 9 aa, Artificial Sequence
  VL CDR3 of Ab57 and Ab146
QQYGSSPRT

SEQ ID NO: 888 - protein, 11 aa, Artificial Sequence
  VL CDR3 of Ab58 (Table 1C)
QRRSNWPPGFT

SEQ ID NO: 889 - protein, 9 aa, Artificial Sequence
  VL CDR3 of Ab59 (Table 1C)
QQTYNTPIT

SEQ ID NO: 890 - protein, 10 aa, Artificial Sequence
  VL CDR3 of Ab60, Ab124 and Ab180
QQYGNSPPIT

SEQ ID NO: 891 - protein, 9 aa, Artificial Sequence
  VL CDR3 of Ab62 (Table 1C)
QQFNSYPLT

SEQ ID NO: 892 - protein, 10 aa, Artificial Sequence
  VL CDR3 of Ab63 (Table 1C)
MQGTHWPPLT

SEQ ID NO: 893 - protein, 10 aa, Artificial Sequence
  VL CDR3 of Ab64 (Table 1C)
QQSYSTPLNT

SEQ ID NO: 894 - protein, 8 aa, Artificial Sequence
  VL CDR3 of Ab65 (Table 1C)
CSYAGTYI

SEQ ID NO: 895 - protein, 10 aa, Artificial Sequence [sequence omitted]

SEQ ID NO: 896 - protein, 9 aa, Artificial Sequence
  VL CDR3 of Ab71 (Table 1C)
QQSYSTLWT

SEQ ID NO: 897 - protein, 9 aa, Artificial Sequence
  VL CDR3 of Ab72 (Table 1C)
ETWDSNTQV

SEQ ID NO: 898 - protein, 8 aa, Artificial Sequence
  VL CDR3 of Ab73 (Table 1C)
QAWDRDVV

SEQ ID NO: 899 - protein, 10 aa, Artificial Sequence
  VL CDR3 of Ab74 (Table 1C)
QKYNSAPPLT

SEQ ID NO: 900 - protein, 8 aa, Artificial Sequence
  VL CDR3 of Ab76 (Table 1C)
QQYDSSRT

SEQ ID NO: 901 - protein, 9 aa, Artificial Sequence
  VL CDR3 of Ab77 (Table 1C)
HQYDSRPRT

SEQ ID NO: 902 - protein, 11 aa, Artificial Sequence
  VL CDR3 of Ab78 (Table 1C)
DSRESSANRVI

SEQ ID NO: 903 - protein, 11 aa, Artificial Sequence
  VL CDR3 of Ab79 (Table 1C)
TSYAGSNNPYV

SEQ ID NO: 904 - protein, 10 aa, Artificial Sequence
  VL CDR3 of Ab81 (Table 1C)
QQSYSTPPFT

SEQ ID NO: 905 - protein, 10 aa, Artificial Sequence
  VL CDR3 of Ab82 (Table 1C)
SSYTTSSTLV

SEQ ID NO: 906 - protein, 10 aa, Artificial Sequence
  VL CDR3 of Ab83 (Table 1C)
QQYGSSRGFT

SEQ ID NO: 907 - protein, 9 aa, Artificial Sequence
  VL CDR3 of Ab84 (Table 1C)
QQAKGFPLT

SEQ ID NO: 908 - protein, 11 aa, Artificial Sequence
  VL CDR3 of Ab86 (Table 1C)
QQRSNWPLALT

SEQ ID NO: 909 - protein, 9 aa, Artificial Sequence
  VL CDR3 of Ab87 (Table 1C)
QQSGRSTRT

SEQ ID NO: 910 - protein, 9 aa, Artificial Sequence
  VL CDR3 of Ab89 (Table 1C)
QQYGRSPFT

SEQ ID NO: 911 - protein, 11 aa, Artificial Sequence
  VL CDR3 of Ab91 (Table 1C)
QQRSNWPPSFT

SEQ ID NO: 912 - protein, 9 aa, Artificial Sequence
  VL CDR3 of Ab92 (Table 1C)
QQSYTIPLT

SEQ ID NO: 913 - protein, 10 aa, Artificial Sequence
  VL CDR3 of Ab93 (Table 1C)
CSYAGSYSYV

SEQ ID NO: 914 - protein, 8 aa, Artificial Sequence
  VL CDR3 of Ab94 (Table 1C)
QQYGSSVT

SEQ ID NO: 915 - protein, 9 aa, Artificial Sequence
  VL CDR3 of Ab95 (Table 1C)
QQTHSFSLS

SEQ ID NO: 916 - protein, 5 aa, Artificial Sequence
  VL CDR3 of Ab96 (Table 1C)
QQSWT

SEQ ID NO: 917 - protein, 9 aa, Artificial Sequence
  VL CDR3 of Ab97 (Table 1C)
QQYNSYPWT

SEQ ID NO: 918 - protein, 9 aa, Artificial Sequence
  VL CDR3 of Ab98 (Table 1C)
QQYNSYSWT

SEQ ID NO: 919 - protein, 8 aa, Artificial Sequence
  VL CDR3 of Ab99 (Table 1C)
QAWDSSPV

SEQ ID NO: 920 - protein, 9 aa, Artificial Sequence
  VL CDR3 of Ab100 (Table 1C)
QQRYNWPPT

SEQ ID NO: 921 - protein, 9 aa, Artificial Sequence
  VL CDR3 of Ab101 (Table 1C)
QQYKNWPQT

SEQ ID NO: 922 - protein, 9 aa, Artificial Sequence
  VL CDR3 of Ab102 (Table 1C)
IQTLQTPYT

SEQ ID NO: 923 - protein, 9 aa, Artificial Sequence
  VL CDR3 of Ab103 (Table 1C)
LQHNSYPLT

SEQ ID NO: 924 - protein, 9 aa, Artificial Sequence
  VL CDR3 of Ab104 (Table 1C)
MQGTHWPWT

SEQ ID NO: 925 - protein, 9 aa, Artificial Sequence
  VL CDR3 of Ab105 (Table 1C)
MQGSYFPLT

SEQ ID NO: 926 - protein, 9 aa, Artificial Sequence
  VL CDR3 of Ab106 (Table 1C)
QQSNRSPLT

SEQ ID NO: 927 - protein, 9 aa, Artificial Sequence
  VL CDR3 of Ab107 (Table 1C)
QHYGSSPGA

SEQ ID NO: 928 - protein, 9 aa, Artificial Sequence
  VL CDR3 of Ab108 (Table 1C)
SSYAGSRWV

SEQ ID NO: 929 - protein, 9 aa, Artificial Sequence
  VL CDR3 of Ab109 (Table 1C)
QHYSGSPWT

SEQ ID NO: 930 - protein, 9 aa, Artificial Sequence
  VL CDR3 of Ab110 (Table 1C)
QQANNFPLT

SEQ ID NO: 931 - protein, 10 aa, Artificial Sequence
  VL CDR3 of Ab111 (Table 1C)
TSYSDTSPYV

SEQ ID NO: 932 - protein, 10 aa, Artificial Sequence
  VL CDR3 of Ab112 (Table 1C)
QLFNRPAPWT

SEQ ID NO: 933 - protein, 8 aa, Artificial Sequence
  VL CDR3 of Ab113 (Table 1C)
QAWDSSTV

SEQ ID NO: 934 - protein, 9 aa, Artificial Sequence
  VL CDR3 of Ab114 (Table 1C)
QQYYRTPLT

SEQ ID NO: 935 - protein, 9 aa, Artificial Sequence
  VL CDR3 of Ab115 (Table 1C)
QTWDTGSVV

SEQ ID NO: 936 - protein, 9 aa, Artificial Sequence
  VL CDR3 of Ab116 (Table 1C)
QQSYSTPGT

SEQ ID NO: 937 - protein, 9 aa, Artificial Sequence
  VL CDR3 of Ab120 (Table 1C)
QQYGRSAWT

SEQ ID NO: 938 - protein, 9 aa, Artificial Sequence
  VL CDR3 of Ab122 (Table 1C)
HQSYSTPFT

SEQ ID NO: 939 - protein, 11 aa, Artificial Sequence
  VL CDR3 of Ab123 (Table 1C)
NSRDISAKHWF

SEQ ID NO: 940 - protein, 9 aa, Artificial Sequence
  VL CDR3 of Ab125 (Table 1C)
QQYNSWPLT

SEQ ID NO: 941 - protein, 9 aa, Artificial Sequence
  VL CDR3 of Ab126 (Table 1C)
QQAKSFPFS

SEQ ID NO: 942 - protein, 10 aa, Artificial Sequence
  VL CDR3 of Ab127 (Table 1C)
SSRTRSGTYV

SEQ ID NO: 943 - protein, 9 aa, Artificial Sequence
  VL CDR3 of Ab129 (Table 1C)
QAWDRSTVV

SEQ ID NO: 944 - protein, 11 aa, Artificial Sequence
  VL CDR3 of Ab130 (Table 1C)
QQRSNWPRALT

SEQ ID NO: 945 - protein, 11 aa, Artificial Sequence
  VL CDR3 of Ab131 (Table 1C)
AAWDDSLSAWV

SEQ ID NO: 946 - protein, 9 aa, Artificial Sequence
  VL CDR3 of Ab132 (Table 1C)
QQYDTYPYT

SEQ ID NO: 947 - protein, 9 aa, Artificial Sequence
  VL CDR3 of Ab133 (Table 1C)
GSYTGTSNV

SEQ ID NO: 948 - protein, 10 aa, Artificial Sequence
  VL CDR3 of Ab134 (Table 1C)
QQRSNWPPWT

SEQ ID NO: 949 - protein, 9 aa, Artificial Sequence
  VL CDR3 of Ab135 (Table 1C)
QTWGRGIVV

SEQ ID NO: 950 - protein, 9 aa, Artificial Sequence
  VL CDR3 of Ab136 (Table 1C)
QAWDSSAVV

SEQ ID NO: 951 - protein, 9 aa, Artificial Sequence
  VL CDR3 of Ab137 (Table 1C)
MQALQTPLT

SEQ ID NO: 952 - protein, 9 aa, Artificial Sequence
  VL CDR3 of Ab138 (Table 1C)
QHYSSYSYT

SEQ ID NO: 953 - protein, 9 aa, Artificial Sequence
  VL CDR3 of Ab139 and Ab159
MQALQTPIT

SEQ ID NO: 954 - protein, 9 aa, Artificial Sequence
  VL CDR3 of Ab141 (Table 1C)
QQRGNWPLT

SEQ ID NO: 955 - protein, 9 aa, Artificial Sequence
  VL CDR3 of Ab142 (Table 1C)
QQYGRSPWT

SEQ ID NO: 956 - protein, 9 aa, Artificial Sequence
  VL CDR3 of Ab144 (Table 1C)
SSYTSSSWV

SEQ ID NO: 957 - protein, 9 aa, Artificial Sequence
  VL CDR3 of Ab145 (Table 1C)
QQYGSSLFT

SEQ ID NO: 958 - protein, 11 aa, Artificial Sequence
  VL CDR3 of Ab147 (Table 1C)
QQRSNWPPKLT

SEQ ID NO: 959 - protein, 9 aa, Artificial Sequence
  VL CDR3 of Ab148 (Table 1C)
QQYGSSPQT

SEQ ID NO: 960 - protein, 7 aa, Artificial Sequence
  VL CDR2 of Ab122 (Table 1C)
TASSLQS

SEQ ID NO: 961 - protein, 17 aa, Artificial Sequence
  VH CDR2 of Ab24 and Ab146
YIRSSGGYTSYADSVKG

SEQ ID NO: 962 - protein, 9 aa, Artificial Sequence
  VL CDR3 of Ab85 (Table 1C)
QQANSFPAT

SEQ ID NO: 963 - protein, 108 aa, Artificial Sequence
  Ab1 VL domain
DIQMTQSPTSLSAFVGDRVTITCQASQDIGNYLNWYQQKSGEPPKLLVYDASFLKKGVPSRFSGSGSGTQYFLTIYSLQPEDFATYFCQHSDNLSVTFGGGTKVEVKR

SEQ ID NO: 964 - protein, 108 aa, Artificial Sequence
  Ab21 VL domain
DIQMTQSPTSLSAFVGDRVTITCQASQDIGNYLNWYQQKSGEPPKLLVYDASFLKKGVPSRFSGSGSGTQYFLTIYSLQPEDFATYFCQHSDSLSVTFGGGTKVEVKR

SEQ ID NO: 965 - protein, 106 aa, Artificial Sequence
  Ab21 light chain constant domain
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 966-968 - skipped (3 entries)

SEQ ID NO: 969 - protein, 9 aa, Artificial Sequence
  VL CDR3 of Ab149 (Table 1C)
QKYNTGLFT

SEQ ID NO: 970 - protein, 9 aa, Artificial Sequence
  VL CDR3 of Ab151 (Table 1C)
QQSYSTPLT

SEQ ID NO: 971 - protein, 9 aa, Artificial Sequence
  VL CDR3 of Ab152 (Table 1C)
QQYGSSRWT

SEQ ID NO: 972 - protein, 6 aa, Artificial Sequence
  VL CDR3 of Ab153 (Table 1C)
QQHASG

SEQ ID NO: 973 - protein, 9 aa, Artificial Sequence
  VL CDR3 of Ab154 (Table 1C)
QQSYSTPPT

SEQ ID NO: 974 - protein, 9 aa, Artificial Sequence
  VL CDR3 of Ab155 (Table 1C)
QQANSFPLT

SEQ ID NO: 975 - protein, 11 aa, Artificial Sequence
  VL CDR3 of Ab156 (Table 1C)
QQSYSTRDLLT

SEQ ID NO: 976 - protein, 9 aa, Artificial Sequence
  VL CDR3 of Ab157 (Table 1C)
QQYGSSLLT

SEQ ID NO: 977 - protein, 10 aa, Artificial Sequence
  VL CDR3 of Ab158 (Table 1C)
SSYTTSNTLV

SEQ ID NO: 978 - protein, 10 aa, Artificial Sequence
  VL CDR3 of Ab160 (Table 1C)
QAWDSSTYVV

SEQ ID NO: 979 - protein, 9 aa, Artificial Sequence
  VL CDR3 of Ab161 (Table 1C)
QAWDSNTVV

SEQ ID NO: 980 - protein, 9 aa, Artificial Sequence
  VL CDR3 of Ab162 (Table 1C)
LQHSTYPLT

SEQ ID NO: 981 - protein, 10 aa, Artificial Sequence
  VL CDR3 of Ab163 (Table 1C)
QAWDTTTFYV

SEQ ID NO: 982 - protein, 9 aa, Artificial Sequence
  VL CDR3 of Ab164 (Table 1C)
QQYYSTPLT

SEQ ID NO: 983 - protein, 10 aa, Artificial Sequence
  VL CDR3 of Ab165 (Table 1C)
AAWDDNVGGV

SEQ ID NO: 984 - protein, 9 aa, Artificial Sequence
  VL CDR3 of Ab166 (Table 1C)
QHRVNGLFI

SEQ ID NO: 985 - protein, 9 aa, Artificial Sequence
  VL CDR3 of Ab167 (Table 1C)
QQSHYIPLT

SEQ ID NO: 986 - protein, 11 aa, Artificial Sequence
  VL CDR3 of Ab168 (Table 1C)
AAWDDSLSGVV

SEQ ID NO: 987 - protein, 9 aa, Artificial Sequence
  VL CDR3 of Ab169 (Table 1C)
QQSFINPQT

SEQ ID NO: 988 - protein, 9 aa, Artificial Sequence
  VL CDR3 of Ab171 (Table 1C)
QQYGRSRYT

SEQ ID NO: 989 - protein, 9 aa, Artificial Sequence
  VL CDR3 of Ab172 (Table 1C)
QQSHSASWT

SEQ ID NO: 990 - protein, 9 aa, Artificial Sequence
  VL CDR3 of Ab173 (Table 1C)
QQYYSTPRT

SEQ ID NO: 991 - protein, 9 aa, Artificial Sequence
  VL CDR3 of Ab174 (Table 1C)
QQYNSYPVT

SEQ ID NO: 992 - protein, 9 aa, Artificial Sequence
  VL CDR3 of Ab175 (Table 1C)
QQANTFPLT

SEQ ID NO: 993 - protein, 9 aa, Artificial Sequence
  VL CDR3 of Ab176 (Table 1C)
QQTYNIVFT

SEQ ID NO: 994 - protein, 11 aa, Artificial Sequence
  VL CDR3 of Ab177 (Table 1C)
AAWDDSLGGPV

SEQ ID NO: 995 - protein, 10 aa, Artificial Sequence
  VL CDR3 of Ab178 (Table 1C)
QQYNNESGGT

SEQ ID NO: 996 - protein, 11 aa, Artificial Sequence
  VL CDR3 of Ab179 (Table 1C)
QQRSNWPPIFT

SEQ ID NO: 997 - protein, 9 aa, Artificial Sequence
  VL CDR3 of Ab181 (Table 1C)
MQALQTPLS

SEQ ID NO: 998 - protein, 9 aa, Artificial Sequence
  VL CDR3 of Ab182 (Table 1C)
QQYGTSRWT

SEQ ID NO: 999 - protein, 9 aa, Artificial Sequence
  VL CDR3 of Ab184 (Table 1C)
QQRSSWPLT

SEQ ID NO: 1000 - protein, 9 aa, Artificial Sequence
  VL CDR3 of Ab186 (Table 1C)
QQANSFPRT

SEQ ID NO: 1001 - protein, 9 aa, Artificial Sequence
  VL CDR3 of Ab187 (Table 1C)
QAWDNSRFI

SEQ ID NO: 1002 - protein, 9 aa, Artificial Sequence
  VL CDR3 of Ab188 (Table 1C)
QQANRFPLT

SEQ ID NO: 1003 - protein, 11 aa, Artificial Sequence
  VL CDR3 of Ab189 (Table 1C)
AAWDDSLSGWV

SEQ ID NO: 1004 - protein, 9 aa, Artificial Sequence
  VL CDR3 of Ab190 (Table 1C)
QQRSNWRIT

SEQ ID NO: 1005 - protein, 13 aa, Artificial Sequence
  VL CDR3 of Ab191 (Table 1C)
NSRDSSGNHLGVV

SEQ ID NO: 1006 - protein, 9 aa, Artificial Sequence
  VL CDR3 of Ab192 (Table 1C)
QQYNVSPYT

SEQ ID NO: 1007 - protein, 11 aa, Artificial Sequence
  VH CDR3 of Ab24 and Ab146 (Table 3C)
APGYYYYGMDV

SEQ ID NO: 1008 - protein, 6 aa, Artificial Sequence
  VH CDR3 of Ab25 (Table 3C)
SRTLDY

SEQ ID NO: 1009 - protein, 11 aa, Artificial Sequence
  VH CDR3 of Ab26 (Table 3C)
ELGPLLGGRGY

SEQ ID NO: 1010 - protein, 11 aa, Artificial Sequence
  VH CDR3 of Ab27 and Ab101
RAAGYGGAFDI

SEQ ID NO: 1011 - protein, 6 aa, Artificial Sequence
  VH CDR3 of Ab28 (Table 3C)
SGNLDY

SEQ ID NO: 1012 - protein, 7 aa, Artificial Sequence
  VH CDR3 of Ab29 (Table 3C)
GPQYFDY

SEQ ID NO: 1013 - protein, 5 aa, Artificial Sequence
  VH CDR3 of Ab30 (Table 3C)
LPPGD

SEQ ID NO: 1014 - protein, 10 aa, Artificial Sequence
  VH CDR3 of Ab31 (Table 3C)
QGGGSFTRDY

SEQ ID NO: 1015 - protein, 8 aa, Artificial Sequence
  VH CDR3 of Ab32 (Table 3C)
GTVGAFDI

SEQ ID NO: 1016 - protein, 8 aa, Artificial Sequence
  VH CDR3 of Ab33 (Table 3C)
DRSGTHDY

SEQ ID NO: 1017 - protein, 11 aa, Artificial Sequence
  VH CDR3 of Ab34 (Table 3C)
DWRVMVNPFDY

SEQ ID NO: 1018 - protein, 10 aa, Artificial Sequence
  VH CDR3 of Ab35 (Table 3C)
PAYSSSPGDY

SEQ ID NO: 1019 - protein, 9 aa, Artificial Sequence
  VH CDR3 of Ab36 (Table 3C)
EAIGRAFDI

SEQ ID NO: 1020 - protein, 8 aa, Artificial Sequence
  VH CDR3 of Ab38 and Ab170
GYGHGLDY

SEQ ID NO: 1021 - protein, 10 aa, Artificial Sequence
  VH CDR3 of Ab39 (Table 3C)
DPMGVVGADY

SEQ ID NO: 1022 - protein, 17 aa, Artificial Sequence
  VH CDR3 of Ab40 (Table 3C)
DMTTVPKLGVYYYGMDV

SEQ ID NO: 1023 - protein, 8 aa, Artificial Sequence
  VH CDR3 of Ab41 (Table 3C)
IVGGAFDI

SEQ ID NO: 1024 - protein, 9 aa, Artificial Sequence
  VH CDR3 of Ab42 (Table 3C)
GRYSYGLDV

SEQ ID NO: 1025 - protein, 12 aa, Artificial Sequence
  VH CDR3 of Ab43 (Table 3C)
DGAGERRGAFDI

SEQ ID NO: 1026 - protein, 10 aa, Artificial Sequence
  VH CDR3 of Ab44 (Table 3C)
VTGYPYLFDY

SEQ ID NO: 1027 - protein, 7 aa, Artificial Sequence
  VH CDR3 of Ab45 (Table 3C)
VGYSKNY

SEQ ID NO: 1028 - protein, 11 aa, Artificial Sequence
  VH CDR3 of Ab46 (Table 3C)
GIAVAGWAFDI

SEQ ID NO: 1029 - protein, 3 aa, Artificial Sequence
  VH CDR3 of Ab47 (Table 3C)
PGP

SEQ ID NO: 1030 - protein, 17 aa, Artificial Sequence
  VH CDR3 of Ab48 (Table 3C)
ERGSSGGVNYYYYGMDV

SEQ ID NO: 1031 - protein, 8 aa, Artificial Sequence
  VH CDR3 of Ab49 (Table 3C)
RYSGSFDY

SEQ ID NO: 1032 - protein, 9 aa, Artificial Sequence
  VH CDR3 of Ab50 (Table 3C)
DGGAHAFDI

SEQ ID NO: 1033 - protein, 3 aa, Artificial Sequence
  VH CDR3 of Ab51 (Table 3C)
QDY

SEQ ID NO: 1034 - protein, 8 aa, Artificial Sequence
  VH CDR3 of Ab52 (Table 3C)
ASVGAFDY

SEQ ID NO: 1035 - protein, 7 aa, Artificial Sequence
  VH CDR3 of Ab53 (Table 3C)
GNVHFDY

SEQ ID NO: 1036 - protein, 13 aa, Artificial Sequence
  VH CDR3 of Ab54 (Table 3C)
RPRVGSTGSVLDS

SEQ ID NO: 1037 - protein, 8 aa, Artificial Sequence
  VH CDR3 of Ab55 (Table 3C)
HSGGAFDI

SEQ ID NO: 1038 - protein, 9 aa, Artificial Sequence
  VH CDR3 of Ab56 (Table 3C)
DRGSYGMDV

SEQ ID NO: 1039 - protein, 22 aa, Artificial Sequence
  VH CDR3 of Ab57 (Table 3C)
GLVTYYYDSSGYYRWGYYGMDV

SEQ ID NO: 1040 - protein, 8 aa, Artificial Sequence
  VH CDR3 of Ab58 (Table 3C)
LTGSYSDY

SEQ ID NO: 1041 - protein, 7 aa, Artificial Sequence
  VH CDR3 of Ab59 (Table 3C)
GIAVARY

SEQ ID NO: 1042 - protein, 11 aa, Artificial Sequence
  VH CDR3 of Ab60 and Ab180
KTIGRARAFDI

SEQ ID NO: 1043 - protein, 7 aa, Artificial Sequence
  VH CDR3 of Ab61 (Table 3C)
GLMGFDY

SEQ ID NO: 1044 - protein, 12 aa, Artificial Sequence
  VH CDR3 of Ab62 (Table 3C)
GYSGSSRGGLRY

SEQ ID NO: 1045 - protein, 6 aa, Artificial Sequence
  VH CDR3 of Ab63 (Table 3C)
FRPEDW

SEQ ID NO: 1046 - protein, 9 aa, Artificial Sequence
  VH CDR3 of Ab64 (Table 3C)
GYGRGAFDI

SEQ ID NO: 1047 - protein, 10 aa, Artificial Sequence
  VH CDR3 of Ab65 (Table 3C)
RGDGNSYLGY

SEQ ID NO: 1048 - protein, 8 aa, Artificial Sequence
  VH CDR3 of Ab66 (Table 3C)
GYSRLFDY

SEQ ID NO: 1049 - protein, 13 aa, Artificial Sequence
  VH CDR3 of Ab70 (Table 3C)
ALSSGWQNNWFDS

SEQ ID NO: 1050 - protein, 10 aa, Artificial Sequence
  VH CDR3 of Ab71 (Table 3C)
GPTVVNHFQH

SEQ ID NO: 1051 - protein, 17 aa, Artificial Sequence
  VH CDR3 of Ab72 (Table 3C)
HLIGRDGYNYYYYGMDV

SEQ ID NO: 1052 - protein, 12 aa, Artificial Sequence
  VH CDR3 of Ab73 (Table 3C)
IPSYWRDYGMDV

SEQ ID NO: 1053 - protein, 6 aa, Artificial Sequence
  VH CDR3 of Ab74 (Table 3C)
HKAFDI

SEQ ID NO: 1054 - protein, 6 aa, Artificial Sequence
  VH CDR3 of Ab75 (Table 3C)
ARGLDV

SEQ ID NO: 1055 - protein, 10 aa, Artificial Sequence
  VH CDR3 of Ab76 (Table 3C)
DKHRYYGMDV

SEQ ID NO: 1056 - protein, 10 aa, Artificial Sequence
  VH CDR3 of Ab77 (Table 3C)
QGGGGRAFDI

SEQ ID NO: 1057 - protein, 12 aa, Artificial Sequence
  VH CDR3 of Ab78 (Table 3C)
GGAAVNTSWFDP

SEQ ID NO: 1058 - protein, 10 aa, Artificial Sequence
  VH CDR3 of Ab79 (Table 3C)
GPVGWTPADY

SEQ ID NO: 1059 - protein, 7 aa, Artificial Sequence
  VH CDR3 of Ab80 (Table 3C)
GYRAFQN

SEQ ID NO: 1060 - protein, 12 aa, Artificial Sequence
  VH CDR3 of Ab81 (Table 3C)
SKWFGVSRGFDY

SEQ ID NO: 1061 - protein, 13 aa, Artificial Sequence
  VH CDR3 of Ab82 (Table 3C)
GSQQLVLVNWFDP

SEQ ID NO: 1062 - protein, 11 aa, Artificial Sequence
  VH CDR3 of Ab83 (Table 3C)
APRISSRYFDL

SEQ ID NO: 1063 - protein, 9 aa, Artificial Sequence
  VH CDR3 of Ab84 (Table 3C)
DRGLSGFGP

SEQ ID NO: 1064 - protein, 9 aa, Artificial Sequence
  VH CDR3 of Ab85 (Table 3C)
VGLGFAVDI

SEQ ID NO: 1065 - protein, 9 aa, Artificial Sequence
  VH CDR3 of Ab86 (Table 3C)
DIGYGVLDP

SEQ ID NO: 1066 - protein, 7 aa, Artificial Sequence
  VH CDR3 of Ab87 (Table 3C)
GNGGFDS

SEQ ID NO: 1067 - protein, 13 aa, Artificial Sequence
  VH CDR3 of Ab88 (Table 3C)
ARRMRSLWEAFDV

SEQ ID NO: 1068 - protein, 13 aa, Artificial Sequence
  VH CDR3 of Ab89 (Table 3C)
SPRGWGTPSFFDY

SEQ ID NO: 1069 - protein, 8 aa, Artificial Sequence
  VH CDR3 of Ab90 (Table 3C)
KYAGYFDY

SEQ ID NO: 1070 - protein, 8 aa, Artificial Sequence
  VH CDR3 of Ab91 (Table 3C)
ETGGGFDY

SEQ ID NO: 1071 - protein, 12 aa, Artificial Sequence
  VH CDR3 of Ab92 (Table 3C)
VAGGVAGISIDY

SEQ ID NO: 1072 - protein, 5 aa, Artificial Sequence
  VH CDR3 of Ab93 (Table 3C)
LGPGY

SEQ ID NO: 1073 - protein, 11 aa, Artificial Sequence
  VH CDR3 of Ab94 (Table 3C)
MGRSSRGTFDI

SEQ ID NO: 1074 - protein, 9 aa, Artificial Sequence
  VH CDR3 of Ab95 (Table 3C)
TAVGRAFDI

SEQ ID NO: 1075 - protein, 7 aa, Artificial Sequence
  VH CDR3 of Ab96 (Table 3C)
KTSAYDY

SEQ ID NO: 1076 - protein, 19 aa, Artificial Sequence
  VH CDR3 of Ab97 (Table 3C)
EGLIYDILTGYVRPKGMDV

SEQ ID NO: 1077 - protein, 6 aa, Artificial Sequence
  VH CDR3 of Ab99 (Table 3C)
GNGMDV

SEQ ID NO: 1078 - protein, 15 aa, Artificial Sequence
  VH CDR3 of Ab100 (Table 3C)
DPNIVLIWGGQAFDI

SEQ ID NO: 1079 - protein, 12 aa, Artificial Sequence
  VH CDR3 of Ab102 (Table 3C)
GNRYSYGHYFDY

SEQ ID NO: 1080 - protein, 14 aa, Artificial Sequence
  VH CDR3 of Ab103 (Table 3C)
VDVRRFYGDYRQDY

SEQ ID NO: 1081 - protein, 13 aa, Artificial Sequence
  VH CDR3 of Ab104 (Table 3C)
LGFCSGGDCHPDV

SEQ ID NO: 1082 - protein, 11 aa, Artificial Sequence
  VH CDR3 of Ab105 (Table 3C)
DAAGGYGTFDI

SEQ ID NO: 1083 - protein, 15 aa, Artificial Sequence
  VH CDR3 of Ab106 (Table 3C)
GRLERRSYYYYGMDV

SEQ ID NO: 1084 - protein, 11 aa, Artificial Sequence
  VH CDR3 of Ab107 (Table 3C)
DVAGRWRGFDY

SEQ ID NO: 1085 - protein, 13 aa, Artificial Sequence
  VH CDR3 of Ab108 (Table 3C)
GAAYSSGYSFVDY

SEQ ID NO: 1086 - protein, 7 aa, Artificial Sequence
  VH CDR3 of Ab109 (Table 3C)
VKPIAAI

SEQ ID NO: 1087 - protein, 9 aa, Artificial Sequence
  VH CDR3 of Ab110 (Table 3C)
RLGGDYFDL

SEQ ID NO: 1088 - protein, 18 aa, Artificial Sequence
  VH CDR3 of Ab111 (Table 3C)
AFRYYDFWSGPTGDAFDI

SEQ ID NO: 1089 - protein, 11 aa, Artificial Sequence
  VH CDR3 of Ab112 (Table 3C)
ASMGWSHPFDY

SEQ ID NO: 1090 - protein, 11 aa, Artificial Sequence
  VH CDR3 of Ab113 (Table 3C)
DPYSGTSYFDF

SEQ ID NO: 1091 - protein, 11 aa, Artificial Sequence
  VH CDR3 of Ab114 (Table 3C)
ESGVFGSAFDL

SEQ ID NO: 1092 - protein, 4 aa, Artificial Sequence
  VH CDR3 of Ab115 (Table 3C)
DLDY

SEQ ID NO: 1093 - protein, 12 aa, Artificial Sequence
  VH CDR3 of Ab116 (Table 3C)
PSSGWYRYGMDV

SEQ ID NO: 1094 - protein, 8 aa, Artificial Sequence
  VH CDR3 of Ab117 (Table 3C)
IVGGGLDL

SEQ ID NO: 1095 - protein, 8 aa, Artificial Sequence
  VH CDR3 of Ab118 (Table 3C)
ISSSSADY

SEQ ID NO: 1096 - protein, 7 aa, Artificial Sequence
  VH CDR3 of Ab119 (Table 3C)
GGRDLDY

SEQ ID NO: 1097 - protein, 12 aa, Artificial Sequence
  VH CDR3 of Ab120 (Table 3C)
TAAAGSRKAFDI

SEQ ID NO: 1098 - protein, 8 aa, Artificial Sequence
  VH CDR3 of Ab121 (Table 3C)
GYGYSIDY

SEQ ID NO: 1099 - protein, 11 aa, Artificial Sequence
  VH CDR3 of Ab122 (Table 3C)
RSGSYGGSFDY

SEQ ID NO: 1100 - protein, 12 aa, Artificial Sequence
  VH CDR3 of Ab124 (Table 3C)
SPDYYGSGYYDY

SEQ ID NO: 1101 - protein, 8 aa, Artificial Sequence
  VH CDR3 of Ab125 (Table 3C)
MVRGVVHY

SEQ ID NO: 1102 - protein, 10 aa, Artificial Sequence
  VH CDR3 of Ab126 (Table 3C)
VAARRYYFDY

SEQ ID NO: 1103 - protein, 9 aa, Artificial Sequence
  VH CDR3 of Ab127 (Table 3C)
GGSGGVSDY

SEQ ID NO: 1104 - protein, 7 aa, Artificial Sequence
  VH CDR3 of Ab128 (Table 3C)
DRIGMDV

SEQ ID NO: 1105 - protein, 13 aa, Artificial Sequence
  VH CDR3 of Ab129 (Table 3C)
LRSSWERWGSVDY

SEQ ID NO: 1106 - protein, 8 aa, Artificial Sequence
  VH CDR3 of Ab130 (Table 3C)
RIGGAFDI

SEQ ID NO: 1107 - protein, 9 aa, Artificial Sequence
  VH CDR3 of Ab131 and Ab189 (Table 3C)
SMKGGHLDY

SEQ ID NO: 1108 - protein, 9 aa, Artificial Sequence
  VH CDR3 of Ab132 (Table 3C)
SGIFWYFDL

SEQ ID NO: 1109 - protein, 16 aa, Artificial Sequence
  VH CDR3 of Ab133 (Table 3C)
AVSSSEERYYYYGMDV

SEQ ID NO: 1110 - protein, 10 aa, Artificial Sequence
  VH CDR3 of Ab134 (Table 3C)
RQVGRRAFDI

SEQ ID NO: 1111 - protein, 8 aa, Artificial Sequence
  VH CDR3 of Ab135 (Table 3C)
VYAGAFDI

SEQ ID NO: 1112 - protein, 5 aa, Artificial Sequence
  VH CDR3 of Ab136 (Table 3C)
HGFDI

SEQ ID NO: 1113 - protein, 11 aa, Artificial Sequence
  VH CDR3 of Ab137 (Table 3C)
PAGDYYGSGSN

SEQ ID NO: 1114 - protein, 8 aa, Artificial Sequence
  VH CDR3 of Ab138 (Table 3C)
AYGGNFDY

SEQ ID NO: 1115 - protein, 11 aa, Artificial Sequence
  VH CDR3 of Ab139 (Table 3C)
VSSYPGYAFDI

SEQ ID NO: 1116 - protein, 12 aa, Artificial Sequence
  VH CDR3 of Ab140 (Table 3C)
GGGRDWPYYFDY

SEQ ID NO: 1117 - protein, 12 aa, Artificial Sequence
  VH CDR3 of Ab141 (Table 3C)
DRPSSNGDYFDY

SEQ ID NO: 1118 - protein, 18 aa, Artificial Sequence
  VH CDR3 of Ab142 (Table 3C)
SITIFGLRKNYYYYGMDV

SEQ ID NO: 1119 - protein, 8 aa, Artificial Sequence
  VH CDR3 of Ab143 (Table 3C)
LYGGNPDY

SEQ ID NO: 1120 - protein, 20 aa, Artificial Sequence
  VH CDR3 of Ab144 (Table 3C)
HGDYYDSSGYYTPSGYGMDV

SEQ ID NO: 1121 - protein, 8 aa, Artificial Sequence
  VH CDR3 of Ab145 (Table 3C)
GLGGYFQH

SEQ ID NO: 1122 - protein, 8 aa, Artificial Sequence
  VH CDR3 of Ab147 (Table 3C)
ESSGSLDY

SEQ ID NO: 1123 - protein, 10 aa, Artificial Sequence
  VH CDR3 of Ab148 (Table 3C)
YGSGKVAFDI

SEQ ID NO: 1124 - protein, 7 aa, Artificial Sequence
  VH CDR3 of Ab149 (Table 3C)
GGGSFDY

SEQ ID NO: 1125 - protein, 14 aa, Artificial Sequence
  VH CDR3 of Ab151 (Table 3C)
DFQSSGWYVYGMDV

SEQ ID NO: 1126 - protein, 8 aa, Artificial Sequence
  VH CDR3 of Ab152 (Table 3C)
GSSNMLHY

SEQ ID NO: 1127 - protein, 15 aa, Artificial Sequence
  VH CDR3 of Ab153 (Table 3C)
AHSSGSYYYYYGMDV

SEQ ID NO: 1128 - protein, 9 aa, Artificial Sequence
  VH CDR3 of Ab154 (Table 3C)
LNYYYGLDV

SEQ ID NO: 1129 - protein, 11 aa, Artificial Sequence
  VH CDR3 of Ab155 (Table 3C)
SMVRGRRGFDY

SEQ ID NO: 1130 - protein, 12 aa, Artificial Sequence
  VH CDR3 of Ab156 (Table 3C)
TPYYGSGYAFDI

SEQ ID NO: 1131 - protein, 8 aa, Artificial Sequence
  VH CDR3 of Ab157 (Table 3C)
VYARQLDF

SEQ ID NO: 1132 - protein, 9 aa, Artificial Sequence
  VH CDR3 of Ab158 (Table 3C)
SYGSGTFDI

SEQ ID NO: 1133 - protein, 6 aa, Artificial Sequence
  VH CDR3 of Ab159 (Table 3C)
GAPFNY

SEQ ID NO: 1134 - protein, 11 aa, Artificial Sequence
  VH CDR3 of Ab160 (Table 3C)
GLVAATRYFQH

SEQ ID NO: 1135 - protein, 6 aa, Artificial Sequence
  VH CDR3 of Ab161 (Table 3C)
SRGLEY

SEQ ID NO: 1136 - protein, 5 aa, Artificial Sequence
  VH CDR3 of Ab162 (Table 3C)
LPGNY

SEQ ID NO: 1137 - protein, 11 aa, Artificial Sequence
  VH CDR3 of Ab163 (Table 3C)
VTPGWERAFDI

SEQ ID NO: 1138 - protein, 7 aa, Artificial Sequence
  VH CDR3 of Ab164 (Table 3C)
GRGWFDL

SEQ ID NO: 1139 - protein, 10 aa, Artificial Sequence
  VH CDR3 of Ab165 (Table 3C)
DGGSGWYLQH

SEQ ID NO: 1140 - protein, 8 aa, Artificial Sequence
  VH CDR3 of Ab166 and Ab171 (Table 3C)
MVRGVVDY

SEQ ID NO: 1141 - protein, 8 aa, Artificial Sequence
  VH CDR3 of Ab167 (Table 3C)
GGRYGMDV

SEQ ID NO: 1142 - protein, 15 aa, Artificial Sequence
  VH CDR3 of Ab168 (Table 3C)
GKRGVSGSRDFMVYY

SEQ ID NO: 1143 - protein, 21 aa, Artificial Sequence
  VH CDR3 of Ab169 (Table 3C)
DSVRYYDSSGYYYTDYYGMDV

SEQ ID NO: 1144 - protein, 8 aa, Artificial Sequence
  VH CDR3 of Ab172 (Table 3C)
RYGYGFDY

SEQ ID NO: 1145 - protein, 8 aa, Artificial Sequence
  VH CDR3 of Ab173 (Table 3C)
VYGGAFDI

SEQ ID NO: 1146 - protein, 8 aa, Artificial Sequence
  VH CDR3 of Ab174 (Table 3C)
VYGGNFDY

SEQ ID NO: 1147 - protein, 11 aa, Artificial Sequence
  VH CDR3 of Ab175 (Table 3C)
HGLGSWYSFDY

SEQ ID NO: 1148 - protein, 10 aa, Artificial Sequence
  VH CDR3 of Ab176 (Table 3C)
WHGGKSAFDY

SEQ ID NO: 1149 - protein, 10 aa, Artificial Sequence
  VH CDR3 of Ab177 (Table 3C)
GPVAGGHFDY

SEQ ID NO: 1150 - protein, 8 aa, Artificial Sequence
  VH CDR3 of Ab178 (Table 3C)
AWGNAFDI

SEQ ID NO: 1151 - protein, 8 aa, Artificial Sequence
  VH CDR3 of Ab179 (Table 3C)
VTTNAFDI

SEQ ID NO: 1152 - protein, 6 aa, Artificial Sequence
  VH CDR3 of Ab181 (Table 3C)
GPAFDI

SEQ ID NO: 1153 - protein, 8 aa, Artificial Sequence
  VH CDR3 of Ab182 (Table 3C)
GTTGAFDI

SEQ ID NO: 1154 - protein, 8 aa, Artificial Sequence
  VH CDR3 of Ab183 (Table 3C)
GSSGSFDY

SEQ ID NO: 1155 - protein, 12 aa, Artificial Sequence
  VH CDR3 of Ab184 (Table 3C)
HYDSSGRGAFDI

SEQ ID NO: 1156 - protein, 5 aa, Artificial Sequence
  VH CDR3 of Ab186 (Table 3C)
GFGDS

SEQ ID NO: 1157 - protein, 4 aa, Artificial Sequence
  VH CDR3 of Ab187 (Table 3C)
NFDN

SEQ ID NO: 1158 - protein, 7 aa, Artificial Sequence
  VH CDR3 of Ab188 (Table 3C)
GGWELPH

SEQ ID NO: 1159 - protein, 8 aa, Artificial Sequence
  VH CDR3 of Ab190 (Table 3C)
FSLGNFDF

SEQ ID NO: 1160 - protein, 10 aa, Artificial Sequence
  VH CDR3 of Ab191 (Table 3C)
VVGGLAPFDY

SEQ ID NO: 1161 - protein, 10 aa, Artificial Sequence
  VH CDR3 of Ab192 (Table 3C)
AGRGAGLATV

SEQ ID NO: 1162 - protein, 108 aa, Artificial Sequence
  Ab167 VL domain
DIQMTQSPSSLSASVGDRVTITCRASQSISTYLNWYQQKPGKAPNLLIYAASSLHTGVPSRFSGSGSGTDFTLTISSLQPEDFATYYCQQSHYIPLTFGGGTKVDIKR

SEQ ID NO: 1163 - protein, 117 aa, Artificial Sequence
  Ab167 VH domain
EVQLLESGGGLVQPGGSLRLSCAASGFTFSDYSMGWVRQAPGKGLEWVSRISSSGGWTGYADSVKGRFTISRDNSKNTLYLQMNSLRAEDTAVYYCARGGRYGMDVWGQGTTVTVSS

SEQ ID NO: 1164 - protein, 108 aa, Artificial Sequence
  Ab63 VL domain
DIQMTQSPSSVSASVGDRVTINCRASQDIRTWLAWYQQKPGKAPKLLIYAASRLQSGVPPRISGSGSGTNFTLTISSLQPGDFATYYCQQAKGFPLTFGGGTKVEIKR

SEQ ID NO: 1165 - protein, 118 aa, Artificial Sequence
  Ab63 VH domain
EVQLLESGGGLVQPGGSLRLSCAASGFTFSYYQMGWVRQAPGKGLEWVSSISSSGGSTIYADSVKGRFTISRDNSKNTLYLQMNSLRAEDTAVYYCARDRGLSGFGPWGQGTLVTVSS

(19) United States
(12) Patent Application Publication
(10) Pub. No.: US 2013/0011406 A1
(43) Pub. Date: Jan. 10, 2013

(54) ANTI-KIT ANTIBODIES AND USES THEREOF

(75) Inventors: Yaron Hadari, Harrison, NY (US); Susanne Radke, Hamden, CT (US); Joseph Schlessinger, Woodbridge, CT (US); Yoshihisa Suzuki, Hamden, CT (US)

(73) Assignee: KOLLTAN PHARMACEUTICALS, INC., New Haven, CT (US)

(21) Appl. No.: 13/637,272

(22) PCT Filed: Mar. 25, 2011
(86) PCT No.: PCT/US11/29980

§ 371 Date: Sep. 25, 2012

Related U.S. Application Data
(60) Provisional application No. 61/426,387, filed on Dec. 22, 2010.
(60) Provisional application No. 61/318,206, filed on Mar. 26, 2010.

(51) Int. Cl. A61K 39/395; C12N 15/13; C12N 15/63; A61P 35/00; A61P 29/00; C12N 1/13; G01N 33/566; C12N 5/071; C12N 1/21; C12N 1/19; C12N 5/10; C07K 16/18; A61P 35/02

(52) U.S. Cl. 424/139.1; 530/387.9; 530/391.7; 424/178.1; 536/23.53; 435/320.1; 435/7.1; 435/375; 435/252.33; 435/252.31; 435/254.23; 435/254.21; 435/331; 435/419; 435/257.2

(57) ABSTRACT

Provided herein, in one aspect, are antibodies that immunospecifically bind to a human KIT antigen comprising the fourth and/or fifth extracellular Ig-like domains (that is, D4 and/or D5 domains), polynucleotides comprising nucleotide sequences encoding such antibodies, and expression vectors and host cells for producing such antibodies. The antibodies can inhibit KIT activity, such as ligand-induced receptor phosphorylation. Also provided herein are kits and pharmaceutical compositions comprising antibodies that specifically bind to a KIT antigen, as well as methods of treating or managing a KIT-mediated disorder or disease and methods of diagnosing a KIT-mediated disorder or disease using the antibodies described herein.

This application claims the benefit of U.S. Provisional Application No. 61/318,206 filed Mar. 26, 2010, and U.S. Provisional Application No. 61/426,387 filed Dec. 22, 2010, each of which is incorporated by reference herein in its entirety.

### 1. INTRODUCTION

Provided herein are antibodies that specifically bind to a KIT antigen, for example a human KIT antigen (e.g., the fourth and fifth extracellular Ig-like domains of human KIT), polynucleotides comprising nucleotide sequences encoding such antibodies, and expression vectors and host cells for producing such antibodies. The antibodies can inhibit KIT activity, such as ligand-induced receptor phosphorylation. Also provided herein are kits and pharmaceutical compositions comprising antibodies that immunospecifically bind to a KIT antigen, as well as methods for treating or managing a KIT-mediated disorder and methods of diagnosing a KIT-mediated disorder using antibodies described herein.

### 2. BACKGROUND

KIT (or c-Kit) is a type III receptor tyrosine kinase encoded by the c-kit gene. KIT comprises five extracellular immunoglobulin (Ig)-like domains, a single transmembrane region, an inhibitory cytoplasmic juxtamembrane domain, and a split cytoplasmic kinase domain separated by a kinase insert segment (see, e.g., Yarden et al., Nature, 1986, 323:226-232; Ullrich and Schlessinger, Cell, 1990, 61:203-212; Clifford et al., J. Biol. Chem., 2003, 278:31461-31464). The human c-kit gene encoding the KIT receptor has been cloned as described by Yarden et al., EMBO J., 1987, 6:3341-3351. KIT is also known as CD117 or stem cell factor receptor (“SCFR”), because it is the receptor for the stem cell factor (“SCF”) ligand (also known as Steel Factor or Kit Ligand). SCF ligand binding to the first three extracellular Ig-like domains of KIT induces receptor dimerization, and thereby activates intrinsic tyrosine kinase activity through the phosphorylation of specific tyrosine residues in the juxtamembrane and kinase domains (see, e.g., Weiss and Schlessinger, Cell, 1998, 94:277-280; Clifford et al., J. Biol. Chem., 2003, 278:31461-31464). Members of the Stat, Src, ERK, and AKT signaling pathways have been shown to be downstream signal transducers of KIT signaling.

The fourth (D4) and fifth (D5) extracellular Ig-like domains of KIT are believed to mediate receptor dimerization (see, e.g., International Patent Application Publication No. WO 2008/153926; Yuzawa et al., Cell, 2007, 130:323-334).

Expression of KIT has been detected in various cell types, such as mast cells, stem cells, brain cells, melanoblasts, ovary cells, and cancer cells (e.g., leukemia cells). Studies of loss-of-function KIT mutations indicate that KIT is important for the normal growth of hematopoietic progenitor cells, mast cells, melanocytes, primordial germ cells, and the interstitial cells of Cajal (see, e.g., Besmer, P., Cum Opin. Cell Biol., 1991, 3:939-946; Lyman et al., Blood, 1998, 91:1101-1134; Ashman, L K., Int. J. Biochem. Cell Biol., 1999, 31:1037-1051; Kitamura et al., Mutat. Res., 2001, 477:165-171; Mol et al., J. Biol. Chem., 2003, 278:31461-31464). Moreover, KIT plays an important role in hematopoiesis, melanogenesis, and gametogenesis (see Ueda et al., Blood, 2002, 99:3342-3349).

Abnormal KIT activity has been implicated in connection with a number of cancers. For example, gain-of-function KIT mutations resulting in SCF-independent, constitutive activation of KIT are found in certain cancer cells and are associated with certain cancers such as leukemia (e.g., chronic myelogenous leukemia) and gastrointestinal stromal tumors (see, e.g., Mol et al., J. Biol. Chem., 2003, 278:31461-31464).

While reagents for modulation of KIT activity have been explored, including, anti-KIT antibodies, small molecule inhibitors of KIT activity (e.g., tyrosine kinase inhibitors such as GLEEVEC®), and small interfering RNA (siRNA), a need continues to exist for additional specific inhibitors of KIT activity.

### 3. SUMMARY

Provided herein, in one aspect, are isolated antibodies that immunospecifically bind to a KIT polypeptide (e.g., a KIT polypeptide containing a D4/D5 region of human KIT) and inhibit a KIT activity, as well as related compositions, reagents and methods.

In one aspect, provided herein is an isolated antibody (e.g., a human antibody or an antigen binding fragment thereof), which immunospecifically binds to a D4/D5 region of human KIT (e.g., SEQ ID NO: 171), comprising:

(A) (i) a variable light (“VL”) chain region comprising a VL CDR1, VL CDR2, and VL CDR3 having the amino acid sequences of SEQ ID NO: 2 (VL CDR1 of Ab1), SEQ ID NO: 14 (VL CDR2 of Ab1), and SEQ ID NO: 26 (VL CDR3 of Ab1), respectively; and (ii) a variable heavy (“VH”) chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively; wherein said antibody comprises 0, 1, or 2 conservative amino acid substitutions in any one of VL CDR1, VL CDR2, VL CDR3, VH CDR1, VH CDR3, and VH CDR3; or

(B) (i) a VL chain region comprising a VL CDR1, VL CDR2, and VL CDR3 having the amino acid sequences of SEQ ID NO: 2 (VL CDR1 of Ab1), SEQ ID NO: 14 (VL CDR2 of Ab1), and SEQ ID NO: 26 (VL CDR3 of Ab1), respectively, wherein said VL chain region comprises 0, 1, or 2 conservative amino acid substitutions in any one of VL CDR1, VL CDR2, or VL CDR3; and (ii) a VH chain region comprising an amino acid sequence that is at least 80% identical to SEQ ID NO: 188. In a specific embodiment, the VH chain region, comprising an amino acid sequence having at least 80% sequence identity to the amino acid sequence of SEQ ID NO: 188, do not comprise amino acid substitutions or alterations in its CDRs. In a specific embodiment, the VH chain region, comprising an amino acid sequence having at least 80% sequence identity to the amino acid sequence of SEQ ID NO: 188, do not comprise amino acid substitutions or alterations in its CDRs, and wherein the VH chain region comprises CDR1, CDR2, and CDR3 having the amino acid sequence of SEQ ID NOs: 86, 98, and 110, respectively. In a specific embodiment, the VH chain region, comprising an amino acid sequence having at least 80% sequence identity to the amino acid sequence of SEQ ID NO: 188, do not comprise amino acid substitutions or alterations in its CDRs, and wherein the VH chain region comprises CDR1, CDR2, and CDR3 having the amino acid sequence of SEQ ID NOs: 337, 338, and 339, respectively. In another specific embodiment, the VH chain region, comprising an amino acid sequence having at least 80% sequence identity to the amino acid sequence of SEQ ID NO: 188, comprises amino acid substitutions or alterations in its framework region only.

In one aspect, provided herein is an isolated antibody (e.g., a human antibody or an antigen binding fragment thereof), which immunospecifically binds to a D4/D5 region of human KIT (e.g., SEQ ID NO: 171), comprising:
(A) a variable light (“VL”) chain region comprising the amino acid sequence of SEQ ID NO: 176 (VL domain of Ab1), and a variable heavy (“VH”) chain region comprising the amino acid sequence of SEQ ID NO: 188 (VH domain of Ab1);
(B) a VL chain region comprising the amino acid sequence of SEQ ID NO: 177 (VL domain of Ab2), optionally the amino acid sequence of SEQ ID NO: 177 starting at the second amino acid residue, and a VH chain region comprising the amino acid sequence of SEQ ID NO: 189 (VH domain of Ab2);
(C) a VL chain region comprising the amino acid sequence of SEQ ID NO: 178 (VL domain of Ab3) and a VH chain region comprising the amino acid sequence of SEQ ID NO: 190 (VH domain of Ab3);
(D) a VL chain region comprising the amino acid sequence of SEQ ID NO: 179 (VL domain of Ab4), optionally the amino acid sequence of SEQ ID NO: 179 starting at the second amino acid residue, and a VH chain region comprising the amino acid sequence of SEQ ID NO: 191 (VH domain of Ab4);
(E) a VL chain region comprising the amino acid sequence of SEQ ID NO: 180 (VL domain of Ab5), optionally the amino acid sequence of SEQ ID NO: 180 starting at the second amino acid residue, and a VH chain region comprising the amino acid sequence of SEQ ID NO: 192 (VH domain of Ab5);
(F) a VL chain region comprising the amino acid sequence of SEQ ID NO: 181 (VL domain of Ab6), optionally the amino acid sequence of SEQ ID NO: 181 starting at the second amino acid residue, and a VH chain region comprising the amino acid sequence of SEQ ID NO: 193 (VH domain of Ab6);
(G) a VL chain region comprising the amino acid sequence of SEQ ID NO: 182 (VL domain of Ab7), optionally the amino acid sequence of SEQ ID NO: 182 starting at the second amino acid residue, and a VH chain region comprising the amino acid sequence of SEQ ID NO: 194 (VH domain of Ab7);
(H) a VL chain region comprising the amino acid sequence of SEQ ID NO: 183 (VL domain of Ab8), optionally the amino acid sequence of SEQ ID NO: 183 starting at the second amino acid residue, and a VH chain region comprising the amino acid sequence of SEQ ID NO: 195 (VH domain of Ab8);
(I) a VL chain region comprising the amino acid sequence of SEQ ID NO: 184 (VL domain of Ab9), optionally the amino acid sequence of SEQ ID NO: 184 starting at the second amino acid residue, and a VH chain region comprising the amino acid sequence of SEQ ID NO: 196 (VH domain of Ab9);
(J) a VL chain region comprising the amino acid sequence of SEQ ID NO: 185 (VL domain of Ab10), optionally the amino acid sequence of SEQ ID NO: 185 starting at the second amino acid residue, and a VH chain region comprising the amino acid sequence of SEQ ID NO: 197 (VH domain of Ab10);
(K) a VL chain region comprising the amino acid sequence of SEQ ID NO: 186 (VL domain of Ab11), optionally the amino acid sequence of SEQ ID NO: 186 starting at the second amino acid residue, and a VH chain region comprising the amino acid sequence of SEQ ID NO: 198 (VH domain of Ab11);
(L) a VL chain region comprising the amino acid sequence of SEQ ID NO: 187 (VL domain of Ab12), optionally the amino acid sequence of SEQ ID NO: 187 starting at the second amino acid residue, and a VH chain region comprising the amino acid sequence of SEQ ID NO: 199 (VH domain of Ab12);
(M) a VL chain region comprising the amino acid sequence of SEQ ID NO: 328 (VL domain of Ab21) and a VH chain region comprising the amino acid sequence of SEQ ID NO: 329 (VH domain of Ab21);
(N) a VL chain region comprising the amino acid sequence of SEQ ID NO: 963 and a VH chain region comprising the amino acid sequence of SEQ ID NO: 188; or
(M) a VL chain region comprising the amino acid sequence of SEQ ID NO: 964 (VL domain of Ab21) and a VH chain region comprising the amino acid sequence of SEQ ID NO: 329 (VH domain of Ab21).
   In a particular embodiment, the VL chain region further comprises an R (Arg) amino acid residue at the C-terminus. In a specific embodiment, the antibody comprises a VL chain region comprising the amino acid sequence of SEQ ID NO: 176 (VL domain of Ab1), optionally starting at the second amino acid position, and a VH chain region comprising the amino acid sequence of SEQ ID NO: 188 (VH domain of Ab1). In a specific embodiment, the antibody comprises a VL chain region comprising the amino acid sequence of SEQ ID NO: 350 or 963 (VL domain of Ab1), and a VH chain region comprising the amino acid sequence of SEQ ID NO: 188 (VH domain of Ab1). In another specific embodiment, the antibody comprises a VL chain region comprising the amino acid sequence of SEQ ID NO: 328 or 964 (VL domain of Ab21) and a VH chain region comprising the amino acid sequence of SEQ ID NO: 329 (VH domain of Ab21).

In a second aspect, provided herein is an isolated antibody (e.g., a human antibody or an antigen binding fragment thereof), which immunospecifically binds to a D4/D5 region of human KIT (e.g., SEQ ID NO: 171), comprising:
(A) (i) a VL chain region comprising a VL CDR1, VL CDR2, and VL CDR3 having the amino acid sequences of SEQ ID NO: 2 (VL CDR1 of Ab1), SEQ ID NO: 14 (VL CDR2 of Ab1), and SEQ ID NO: 26 (VL CDR3 of Ab1), respectively; and (ii) a VH chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively;
(B) (i) a VL chain region comprising a VL CDR1, VL CDR2, and VL CDR3 having the amino acid sequences of SEQ ID NO: 3 (VL CDR1 of Ab2), SEQ ID NO: 15 (VL CDR2 of Ab2), and SEQ ID NO: 27 (VL CDR3 of Ab2), respectively; and (ii) a VH chain region comprising a VH CDR1, VH CDR2, and VH CDR3 having the amino acid sequences of SEQ ID NO: 87 (VH CDR1 of Ab2), SEQ ID NO: 99 (VH CDR2 of Ab2), and SEQ ID NO: 111 (VH CDR3 of Ab2), respectively;
(C) (i) a VL chain region comprising a VL CDR1, VL CDR2, and VL CDR3 having the amino acid sequences of SEQ ID NO: 4 (VL CDR1 of Ab3), SEQ ID NO: 16 (VL CDR2 of Ab3), and SEQ ID NO: 28 (VL CDR3 of Ab3), respectively; and (ii) a VH chain region comprising a VH CDR1, VH CDR2, and VH CDR3 having the amino acid sequences of SEQ ID NO: 88 (VH CDR1 of Ab3), SEQ ID NO: 100 (VH CDR2 of Ab3), and SEQ ID NO: 112 (VH CDR3 of Ab3), respectively;
(D) (i) a VL chain region comprising a VL CDR1, VL CDR2, and VL CDR3 having the amino acid sequences of SEQ ID NO: 5 (VL CDR1 of Ab4), SEQ ID NO: 17 (VL CDR2 of Ab4), and SEQ ID NO: 29 (VL CDR3 of Ab4), respectively; and (ii) a VH chain region comprising a VH CDR1, VH CDR2, and VH CDR3 having the amino acid sequences of SEQ ID NO: 89 (VH CDR1 of Ab4), SEQ ID NO: 101 (VH CDR2 of Ab4), and SEQ ID NO: 113 (VH CDR3 of Ab4), respectively;
(E) (i) a VL chain region comprising a VL CDR1, VL CDR2, and VL CDR3 having the amino acid sequences of SEQ ID NO: 6 (VL CDR1 of Ab5), SEQ ID NO: 18 (VL CDR2 of Ab5), and SEQ ID NO: 30 (VL CDR3 of Ab5), respectively; and (ii) a VH chain region comprising a VH CDR1, VH CDR2, and VH CDR3 having the amino acid sequences of SEQ ID NO: 90 (VH CDR1 of Ab5), SEQ ID NO: 102 (VH CDR2 of Ab5), and SEQ ID NO: 114 (VH CDR3 of Ab5), respectively.
(F) (i) a VL chain region comprising a VL CDR1, VL CDR2, and VL CDR3 having the amino acid sequences of SEQ ID NO: 7 (VL CDR1 of Ab6), SEQ ID NO: 19 (VL CDR2 of Ab6), and SEQ ID NO: 31 (VL CDR3 of Ab6), respectively; and (ii) a VH chain region comprising a VH CDR1, VH CDR2, and VH CDR3 having the amino acid sequences of SEQ ID NO: 91 (VH CDR1 of Ab6), SEQ ID NO: 103 (VH CDR2 of Ab6), and SEQ ID NO: 115 (VH CDR3 of Ab6), respectively;
(G) (i) a VL chain region comprising a VL CDR1, VL CDR2, and VL CDR3 having the amino acid sequences of SEQ ID NO: 8 (VL CDR1 of Ab7), SEQ ID NO: 20 (VL CDR2 of Ab7), and SEQ ID NO: 32 (VL CDR3 of Ab7), respectively; and (ii) a VH chain region comprising a VH CDR1, VH CDR2, and VH CDR3 having the amino acid sequences of SEQ ID NO: 92 (VH CDR1 of Ab7), SEQ ID NO: 104 (VH CDR2 of Ab7), and SEQ ID NO: 116 (VH CDR3 of Ab7), respectively;
(H) (i) a VL chain region comprising a VL CDR1, VL CDR2, and VL CDR3 having the amino acid sequences of SEQ ID NO: 9 (VL CDR1 of Ab8), SEQ ID NO: 21 (VL CDR2 of Ab8), and SEQ ID NO: 33 (VL CDR3 of Ab8), respectively; and (ii) a VH chain region comprising a VH CDR1, VH CDR2, and VH CDR3 having the amino acid sequences of SEQ ID NO: 93 (VH CDR1 of Ab8), SEQ ID NO: 105 (VH CDR2 of Ab8), and SEQ ID NO: 117 (VH CDR3 of Ab8), respectively;
(I) (i) a VL chain region comprising a VL CDR1, VL CDR2, and VL CDR3 having the amino acid sequences of SEQ ID NO: 10 (VL CDR1 of Ab9), SEQ ID NO: 22 (VL CDR2 of Ab9), and SEQ ID NO: 34 (VL CDR3 of Ab9), respectively; and (ii) a VH chain region comprising a VH CDR1, VH CDR2, and VH CDR3 having the amino acid sequences of SEQ ID NO: 94 (VH CDR1 of Ab9), SEQ ID NO: 106 (VH CDR2 of Ab9), and SEQ ID NO: 118 (VH CDR3 of Ab9), respectively;
(J) (i) a VL chain region comprising a VL CDR1, VL CDR2, and VL CDR3 having the amino acid sequences of SEQ ID NO: 11 (VL CDR1 of Ab10), SEQ ID NO: 23 (VL CDR2 of Ab10), and SEQ ID NO: 35 (VL CDR3 of Ab10), respectively; and (ii) a VH chain region comprising a VH CDR1, VH CDR2, and VH CDR3 having the amino acid sequences of SEQ ID NO: 95 (VH CDR1 of Ab10), SEQ ID NO: 107 (VH CDR2 of Ab10), and SEQ ID NO: 119 (VH CDR3 of Ab10), respectively;
(K) (i) a VL chain region comprising a VL CDR1, VL CDR2, and VL CDR3 having the amino acid sequences of SEQ ID NO: 12 (VL CDR1 of Ab11), SEQ ID NO: 24 (VL CDR2 of Ab11), and SEQ ID NO: 36 (VL CDR1 of Ab11), respectively; and (ii) a VH chain region comprising a VH CDR1, VH CDR2, and VH CDR3 having the amino acid sequences of SEQ ID NO: 96 (VH CDR1 of Ab11), SEQ ID NO: 108 (VH CDR2 of Ab11), and SEQ ID NO: 120 (VH CDR3 of Ab11), respectively;
(L) (i) a VL chain region comprising a VL CDR1, VL CDR2, and VL CDR3 having the amino acid sequences of SEQ ID NO: 13 (VL CDR1 of Ab12), SEQ ID NO: 25 (VL CDR2 of Ab12), and SEQ ID NO: 37 (VL CDR3 of Ab12), respectively; and (ii) a VH chain region comprising a VH CDR1, VH CDR2, and VH CDR3 having the amino acid sequences of SEQ ID NO: 97 (VH CDR1 of Ab12), SEQ ID NO: 109 (VH CDR2 of Ab12), and SEQ ID NO: 121 (VH CDR3 of Ab12), respectively; or
(M) (i) a VL chain region comprising a VL CDR1, VL CDR2, and VL CDR3 having the amino acid sequences of SEQ ID NO: 330 (VL CDR1 of Ab21), SEQ ID NO: 331 (VL CDR2 of Ab21), and SEQ ID NO: 332 (VL CDR3 of Ab21), respectively; and (ii) a VH chain region comprising a VH CDR1, VH CDR2, and VH CDR3 having the amino acid sequences of SEQ ID NO: 337 (VH CDR1 of Ab21), SEQ ID NO: 338 (VH CDR2 of Ab21), and SEQ ID NO: 339 (VH CDR3 of Ab21), respectively.
   In a specific embodiment, the antibody comprises 0, 1, or 2 conservative amino acid substitutions in any one of VL CDR1, VL CDR2, VL CDR3, VH CDR1, VH CDR3, and VH CDR3. In a particular embodiment, the antibody comprises (i) a VL chain region comprising a VL CDR1, VL CDR2, and VL CDR3 having the amino acid sequences of SEQ ID NO: 2 (VL CDR1 of Ab1), SEQ ID NO: 14 (VL CDR2 of Ab1), and SEQ ID NO: 26 (VL CDR3 of Ab1), respectively; and (ii) a VH chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively. In another particular embodiment, the antibody comprises (i) a VL chain region comprising a VL CDR1, VL CDR2, and VL CDR3 having the amino acid sequences of SEQ ID NO: 330 (VL CDR1 of Ab21), SEQ ID NO: 331 (VL CDR2 of Ab21), and SEQ ID NO: 332 (VL CDR3 of Ab21), respectively; and (ii) a VH chain region comprising a VH CDR1, VH CDR2, and VH CDR3 having the amino acid sequences of SEQ ID NO: 337 (VH CDR1 of Ab21), SEQ ID NO: 338 (VH CDR2 of Ab21), and SEQ ID NO: 339 (VH CDR3 of Ab21), respectively.

In a third aspect, provided herein is an isolated antibody (e.g., a human antibody or an antigen binding fragment thereof), which immunospecifically binds to a D4/D5 region of human KIT (e.g., SEQ ID NO: 171), comprising:
(A) a VL chain region comprising a VL CDR1, VL CDR2, and VL CDR3 having the amino acid sequences of SEQ ID NO: 2 (VL CDR1 of Ab1), SEQ ID NO: 14 (VL CDR2 of Ab1), and SEQ ID NO: 26 (VL CDR3 of Ab1), respectively; and a VH chain region comprising the amino acid sequence of SEQ ID NO: 188 (Ab1);
(B) a VL chain region comprising a VL CDR1, VL CDR2, and VL CDR3 having the amino acid sequences of SEQ ID NO: 3 (VL CDR1 of Ab2), SEQ ID NO: 15 (VL CDR2 of Ab2), and SEQ ID NO: 27 (VL CDR3 of Ab2), respectively; and a VH chain region comprising the amino acid sequence of SEQ ID NO: 189 (Ab2);
(C) a VL chain region comprising a VL CDR1, VL CDR2, and VL CDR3 having the amino acid sequences of SEQ ID NO: 4 (VL CDR1 of Ab3), SEQ ID NO: 16 (VL CDR2 of Ab3), and SEQ ID NO: 28 (VL CDR3 of Ab3), respectively; and a VH chain region comprising the amino acid sequence of SEQ ID NO: 190 (Ab3);
(D) a VL chain region comprising a VL CDR1, VL CDR2, and VL CDR3 having the amino acid sequences of SEQ ID NO: 5 (VL CDR1 of Ab4), SEQ ID NO: 17 (VL CDR2 of Ab4), and SEQ ID NO: 29 (VL CDR3 of Ab4), respectively; and a VH chain region comprising the amino acid sequence of SEQ ID NO: 191 (Ab4);
(E) a VL chain region comprising a VL CDR1, VL CDR2, and VL CDR3 having the amino acid sequences of SEQ ID NO: 6 (VL CDR1 of Ab5), SEQ ID NO: 18 (VL CDR2 of Ab5), and SEQ ID NO: 30 (VL CDR3 of Ab5), respectively; and a VH chain region comprising the amino acid sequence of SEQ ID NO: 192 (Ab5);
(F) a VL chain region comprising a VL CDR1, VL CDR2, and VL CDR3 having the amino acid sequences of SEQ ID NO: 7 (VL CDR1 of Ab6), SEQ ID NO: 19 (VL CDR2 of Ab6), and SEQ ID NO: 31 (VL CDR3 of Ab6), respectively; and a VH chain region comprising the amino acid sequence of SEQ ID NO: 193 (Ab6);
(G) a VL chain region comprising a VL CDR1, VL CDR2, and VL CDR3 having the amino acid sequences of SEQ ID NO: 8 (VL CDR1 of Ab7), SEQ ID NO: 20 (VL CDR2 of Ab7), and SEQ ID NO: 32 (VL CDR3 of Ab7), respectively; and a VH chain region comprising the amino acid sequence of SEQ ID NO: 194 (Ab7);
(H) a VL chain region comprising a VL CDR1, VL CDR2, and VL CDR3 having the amino acid sequences of SEQ ID NO: 9 (VL CDR1 of Ab8), SEQ ID NO: 21 (VL CDR2 of Ab8), and SEQ ID NO: 33 (VL CDR3 of Ab8), respectively; and a VH chain region comprising the amino acid sequence of SEQ ID NO: 195 (Ab8);
(I) a VL chain region comprising a VL CDR1, VL CDR2, and VL CDR3 having the amino acid sequences of SEQ ID NO: 10 (VL CDR1 of Ab9), SEQ ID NO: 22 (VL CDR2 of Ab9), and SEQ ID NO: 34 (VL CDR3 of Ab9), respectively; and a VH chain region comprising the amino acid sequence of SEQ ID NO: 196 (Ab9);
(J) a VL chain region comprising a VL CDR1, VL CDR2, and VL CDR3 having the amino acid sequences of SEQ ID NO: 11 (VL CDR1 of Ab10), SEQ ID NO: 23 (VL CDR2 of Ab10), and SEQ ID NO: 35 (VL CDR3 of Ab10), respectively; and a VH chain region comprising the amino acid sequence of SEQ ID NO: 197 (Ab10);
(K) a VL chain region comprising a VL CDR1, VL CDR2, and VL CDR3 having the amino acid sequences of SEQ ID NO: 12 (VL CDR1 of Ab11), SEQ ID NO: 24 (VL CDR2 of Ab11), and SEQ ID NO: 36 (VL CDR3 of Ab11), respectively; and a VH chain region comprising the amino acid sequence of SEQ ID NO: 198 (Ab11);
(L) a VL chain region comprising a VL CDR1, VL CDR2, and VL CDR3 having the amino acid sequences of SEQ ID NO: 13 (VL CDR1 of Ab12), SEQ ID NO: 25 (VL CDR2 of Ab12), and SEQ ID NO: 37 (VL CDR3 of Ab12), respectively; and a VH chain region comprising the amino acid sequence of SEQ ID NO: 199 (Ab12); or
(M) a VL chain region comprising a VL CDR1, VL CDR2, and VL CDR3 having the amino acid sequences of SEQ ID NO: 330 (VL CDR1 of Ab21), SEQ ID NO: 331 (VL CDR2 of Ab21), and SEQ ID NO: 332 (VL CDR3 of Ab21), respectively; and a VH chain region comprising the amino acid sequence of SEQ ID NO: 329 (Ab21). In a specific embodiment, the antibody comprises a VL chain region comprising a VL CDR1, VL CDR2, and VL CDR3 having the amino acid sequences of SEQ ID NO: 2 (VL CDR1 of Ab1), SEQ ID NO: 14 (VL CDR2 of Ab1), and SEQ ID NO: 26 (VL CDR3 of Ab1), respectively; and a VH chain region comprising the amino acid sequence of SEQ ID NO: 188 (Ab1). In another specific embodiment, the antibody comprises a VL chain region comprising a VL CDR1, VL CDR2, and VL CDR3 having the amino acid sequences of SEQ ID NO: 330 (VL CDR1 of Ab21), SEQ ID NO: 331 (VL CDR2 of Ab21), and SEQ ID NO: 332 (VL CDR3 of Ab21), respectively; and a VH chain region comprising the amino acid sequence of SEQ ID NO: 329 (Ab21).

In a fourth aspect, provided herein is an isolated antibody (e.g., a human antibody or an antigen binding fragment thereof), which immunospecifically binds to a D4/D5 region of human KIT (e.g., SEQ ID NO: 171), comprising:
(A) a VL chain region comprising the amino acid sequence of SEQ ID NO: 176 (Ab1); and a VH chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively;
(B) a VL chain region comprising the amino acid sequence of SEQ ID NO: 177 (Ab2); and a VH chain region comprising a VH CDR1, VH CDR2, and VH CDR3 having the amino acid sequences of SEQ ID NO: 87 (VH CDR1 of Ab2), SEQ ID NO: 99 (VH CDR2 of Ab2), and SEQ ID NO: 111 (VH CDR3 of Ab2), respectively;
(C) a VL chain region comprising the amino acid sequence of SEQ ID NO: 178 (Ab3); and a VH chain region comprising a VH CDR1, VH CDR2, and VH CDR3 having the amino acid sequences of SEQ ID NO: 88 (VH CDR1 of Ab3), SEQ ID NO: 100 (VH CDR2 of Ab3), and SEQ ID NO: 112 (VH CDR3 of Ab3), respectively;
(D) a VL chain region comprising the amino acid sequence of SEQ ID NO: 179 (Ab4); and a VH chain region comprising a VH CDR1, VH CDR2, and VH CDR3 having the amino acid sequences of SEQ ID NO: 89 (VH CDR1 of Ab4), SEQ ID NO: 101 (VH CDR2 of Ab4), and SEQ ID NO: 113 (VH CDR3 of Ab4), respectively;
(E) a VL chain region comprising the amino acid sequence of SEQ ID NO: 180 (Ab5); and a VH chain region comprising a VH CDR1, VH CDR2, and VH CDR3 having the amino acid sequences of SEQ ID NO: 90 (VH CDR1 of Ab5), SEQ ID NO: 102 (VH CDR2 of Ab5), and SEQ ID NO: 114 (VH CDR3 of Ab5), respectively;
(F) a VL chain region comprising the amino acid sequence of SEQ ID NO: 181 (Ab6); and a VH chain region comprising a VH CDR1, VH CDR2, and VH CDR3 having the amino acid sequences of SEQ ID NO: 91 (VH CDR1 of Ab6), SEQ ID NO: 103 (VH CDR2 of Ab6), and SEQ ID NO: 115 (VH CDR3 of Ab6), respectively;
(G) a VL chain region comprising the amino acid sequence of SEQ ID NO: 182 (Ab7); and a VH chain region comprising a VH CDR1, VH CDR2, and VH CDR3 having the amino acid sequences of SEQ ID NO: 92 (VH CDR1 of Ab7), SEQ ID NO: 104 (VH CDR2 of Ab7), and SEQ ID NO: 116 (VH CDR3 of Ab7), respectively;
(H) a VL chain region comprising the amino acid sequence of SEQ ID NO: 183 (Ab8); and a VH chain region comprising a VH CDR1, VH CDR2, and VH CDR3 having the amino acid sequences of SEQ ID NO: 93 (VH CDR1 of Ab8), SEQ ID NO: 105 (VH CDR2 of Ab8), and SEQ ID NO: 117 (VH CDR3 of Ab8), respectively;
(I) a VL chain region comprising the amino acid sequence of SEQ ID NO: 184 (Ab9); and a VH chain region comprising a VH CDR1, VH CDR2, and VH CDR3 having the amino acid sequences of SEQ ID NO: 94 (VH CDR1 of Ab9), SEQ ID NO: 106 (VH CDR2 of Ab9), and SEQ ID NO: 118 (VH CDR3 of Ab9), respectively;
(J) a VL chain region comprising the amino acid sequence of SEQ ID NO: 185 (Ab10); and a VH chain region comprising a VH CDR1, VH CDR2, and VH CDR3 having the amino acid sequences of SEQ ID NO: 95 (VH CDR1 of Ab10), SEQ ID NO: 107 (VH CDR2 of Ab10), and SEQ ID NO: 119 (VH CDR3 of Ab10), respectively;
(K) a VL chain region comprising the amino acid sequence of SEQ ID NO: 186 (Ab11); and a VH chain region comprising a VH CDR1, VH CDR2, and VH CDR3 having the amino acid sequences of SEQ ID NO: 96 (VH CDR1 of Ab11), SEQ ID NO: 108 (VH CDR2 of Ab11), and SEQ ID NO: 120 (VH CDR3 of Ab11), respectively;
(L) a VL chain region comprising the amino acid sequence of SEQ ID NO: 187 (Ab12); and a VH chain region comprising a VH CDR1, VH CDR2, and VH CDR3 having the amino acid sequences of SEQ ID NO: 97 (VH CDR1 of Ab12), SEQ ID NO: 109 (VH CDR2 of Ab12), and SEQ ID NO: 121 (VH CDR3 of Ab12), respectively; or
(M) a VL chain region comprising the amino acid sequence of SEQ ID NO: 328 (Ab21); and a VH chain region comprising a VH CDR1, VH CDR2, and VH CDR3 having the amino acid sequences of SEQ ID NO: 337 (VH CDR1 of Ab21), SEQ ID NO: 338 (VH CDR2 of Ab21), and SEQ ID NO: 339 (VH CDR3 of Ab21), respectively.
   In a specific embodiment, the antibody comprises a VL chain region comprising the amino acid sequence of SEQ ID NO: 176 (Ab1), optionally starting at the second amino acid position; and a VH chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively. In a specific embodiment, the antibody comprises a VL chain region comprising the amino acid sequence of SEQ ID NO: 350 (Ab1), optionally starting at the second amino acid position; and a VH chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively. In a specific embodiment, the antibody comprises a VL chain region comprising the amino acid sequence of SEQ ID NO: 9 (Ab1), optionally starting at the second amino acid position; and a VH chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively. In another specific embodiment, the antibody comprises a VL chain region comprising the amino acid sequence of SEQ ID NO: 328 (Ab21); and a VH chain region comprising a VH CDR1, VH CDR2, and VH CDR3 having the amino acid sequences of SEQ ID NO: 337 (VH CDR1 of Ab21), SEQ ID NO: 338 (VH CDR2 of Ab21), and SEQ ID NO: 339 (VH CDR3 of Ab21), respectively. In another specific embodiment, the antibody comprises a VL chain region comprising the amino acid sequence of SEQ ID NO: 964 (Ab21); and a VH chain region comprising a VH CDR1, VH CDR2, and VH CDR3 having the amino acid sequences of SEQ ID NO: 337 (VH CDR1 of Ab21), SEQ ID NO: 338 (VH CDR2 of Ab21), and SEQ ID NO: 339 (VH CDR3 of Ab21), respectively.

In certain embodiments, antibodies described herein comprise a human light chain constant region and a human heavy chain constant region. In further embodiments, the human light chain constant region is a human kappa light chain constant region. In another embodiment, the human heavy chain constant region is a human gamma heavy chain constant region. In certain embodiments, the antibody is an IgG1 isotype f antibody. In particular embodiments, the human light chain constant region comprises the amino acid sequence of SEQ ID NO: 344. In particular embodiments, the human light chain constant region comprises the amino acid sequence of SEQ ID NO: 965. In specific embodiments, the human heavy chain constant region comprises the amino acid sequence of SEQ ID NO: 345. In certain embodiments, an antibody described herein comprises (i) a human light chain constant region comprising the amino acid sequence of SEQ ID NO: 344, (ii) a variable light chain region comprising the amino acid sequence of SEQ ID NO: 328, (iii) a human heavy chain constant region comprising the amino acid sequence of SEQ ID NO: 345, and (iv) a variable heavy chain region comprising the amino acid sequence of SEQ ID NO: 329. In specific embodiments, the human heavy chain constant region comprises the amino acid sequence of SEQ ID NO: 345. In certain embodiments, an antibody described herein comprises (i) a human light chain constant region comprising the amino acid sequence of SEQ ID NO: 965, (ii) a variable light chain region comprising the amino acid sequence of SEQ ID NO: 964, (iii) a human heavy chain constant region comprising the amino acid sequence of SEQ ID NO: 345, and (iv) a variable heavy chain region comprising the amino acid sequence of SEQ ID NO: 329. In certain embodiments, an antibody described herein comprises (i) a human light chain constant region comprising the amino acid sequence of SEQ ID NO: 344, (ii) a variable light chain region comprising the amino acid sequence of SEQ ID NO: 176, (iii) a human heavy chain constant region comprising the amino acid sequence of SEQ ID NO: 345, and (iv) a variable heavy chain region comprising the amino acid sequence of SEQ ID NO: 188. In certain embodiments, an antibody described herein comprises (i) a human light chain constant region comprising the amino acid sequence of SEQ ID NO: 344, (ii) a variable light chain region comprising the amino acid sequence of SEQ ID NO: 176 starting with the second amino acid residue of SEQ ID NO: 176, (iii) a human heavy chain constant region comprising the amino acid sequence of SEQ ID NO: 345, and (iv) a variable heavy chain region comprising the amino acid sequence of SEQ ID NO: 188. In certain embodiments, an antibody described herein comprises (i) a human light chain constant region comprising the amino acid sequence of SEQ ID NO: 965, (ii) a variable light chain region comprising the amino acid sequence of SEQ ID NO: 963, (iii) a human heavy chain constant region comprising the amino acid sequence of SEQ ID NO: 345, and (iv) a variable heavy chain region comprising the amino acid sequence of SEQ ID NO: 188. In a specific embodiment, an antibody described herein comprises a light chain and a heavy chain, wherein the light chain comprises the amino acid sequence:

(SEQ ID NO: 346; of Ab1)
DIQMTQSPTSLSAFVGDRVTITCQASQDIGNYLNWYQQKSGEPPKLLVYD
ASFLKKGVPSRFSGSGSGTQYFLTIYSLQPEDFATYFCQHSDNLSVTFGG
GTKVEVKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC;
and

wherein the heavy chain comprises the amino acid sequence

(SEQ ID NO: 347; of Ab1)
EVQLLESGGGLVQPGGSLRLSCAASGFTFSNYLMSWVRQAPGKGLEWVSS
IVPSGGFTHYADSVKGRFTISRDNSKNTLYLQMNSLRAEDTAVYYCARLQ
TGSWRVHAFDIWGQGTMVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSP
GK.

In a specific embodiment, an antibody described herein comprises a light chain and a heavy chain, wherein the light chain comprises the amino acid sequence:

(SEQ ID NO: 348; of Ab21)
DIQMTQSPTSLSAFVGDRVTITCQASQDIGNYLNWYQQKSGEPPKLLVYD
ASFLKKGVPSRFSGSGSGTQYFLTIYSLQPEDFATYFCQHSDSLSVTFGG
GTKVEVKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC;
and

wherein the heavy chain comprises the amino acid sequence

(SEQ ID NO: 349; of Ab21)
EVQLLESGGGLVQPGGSLRLSCAASGFTFSNYLMSWVRQAPGKGLEWVSS
IVPSGGFTHYADSVKGRFTISRDNSKNTLYLQMNSLRAEDTAVYYCARLQ
TGSWRVHAFDIWGQGTMVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSP
GK.

In a particular embodiment, the isolated antibody described herein is a monoclonal antibody, e.g., a human monoclonal antibody. In another particular embodiment, the isolated antibody described herein is an antigen binding fragment, for example, a Fab antibody. In yet another particular embodiment, the isolated antibody described herein is a human IgG1 or IgG4 antibody. In yet another particular embodiment, the isolated antibody described herein is an inhibitor of KIT activity. In a further embodiment, the isolated antibody described herein which is an inhibitor of KIT activity, inhibits KIT receptor phosphorylation by at least 25% as determined by a solid phase ELISA assay. In one embodiment, the antibody described herein which is an inhibitor of KIT activity inhibits KIT receptor phosphorylation by 25% to 80% as determined by a solid phase ELISA assay. In another embodiment, the antibody described herein which is an inhibitor of KIT activity inhibits KIT receptor phosphorylation by at least 50% as determined by a solid phase ELISA assay. In yet another embodiment, an antibody described herein which is an inhibitor of KIT activity does not block KIT ligand binding to the KIT receptor. In a further embodiment, the isolated antibody described herein which is an inhibitor of KIT activity does not inhibit KIT receptor dimerization. In yet a further embodiment, the isolated antibody described herein which is an inhibitor of KIT activity enhances KIT receptor internalization or KIT receptor degradation. In another embodiment, isolated antibody described herein which is an inhibitor of KIT activity induces apoptosis when a cell expressing KIT is contacted with an effective amount of the isolated antibody.

In a seventh aspect, provided herein are one or more polynucleotides (or isolated polynucleotides) comprising nucleotide sequences encoding an antibody described herein (or an antigen binding fragment thereof). In certain embodiments, a polynucleotide (or isolated polynucleotide) comprises nucleotide sequences encoding a VH chain region and/or a VL chain region, wherein the VH chain region comprises an amino acid sequence of any one of SEQ ID NOs: 188-199, and wherein the VL chain region comprises an amino acid sequence of any one of SEQ ID NOs: 176-187, 350, 963, or 964. In certain embodiments, a polynucleotide (or isolated polynucleotide) comprises nucleotide sequences encoding a heavy chain and/or a light chain, wherein the heavy chain comprises a VH chain region comprising an amino acid sequence of any one of SEQ ID NOs: 188-199, and wherein the light chain comprises a VL chain region comprises an amino acid sequence of any one of SEQ ID NOs: 176-187, 350, 963, or 964.

In a eighth aspect, provided herein is a mammalian expression vector comprising one or more polynucleotides (or isolated polynucleotides) comprising nucleotide sequences encoding an antibody described herein.

In a ninth aspect, provided herein is a host cell comprising a mammalian expression vector described herein.

In an tenth aspect, provided herein is a pharmaceutical composition comprising an isolated antibody described herein (or an antigen binding fragment thereof) and a pharmaceutically acceptable carrier.

In a eleven aspect, provided herein is a kit comprising an isolated antibody described herein.

In a twelfth aspect, provided herein is a method for treating or managing a KIT-mediated disorder or disease in a subject in need thereof, comprising administering to the subject a therapeutically effective amount of an isolated antibody described herein. In a particular embodiment, the KIT-mediated disorder or disease is cancer, an inflammatory condition, or fibrosis. In another particular embodiment, the cancer is leukemia, chronic myelogenous leukemia, lung cancer, small cell lung cancer, or gastrointestinal stromal tumors. In yet another particular embodiment, the cancer is refractory to treatment by a tyrosine kinase inhibitor. In a further particular embodiment, the tyrosine kinase inhibitor is GLEEVEC® or SUTENT®.

In an thirteenth aspect, provided herein is a method for diagnosing a subject with a KIT-mediated disorder or disease comprising contacting a sample obtained from the subject with an isolated antibody described herein and detecting the expression level of KIT in the sample. In a particular embodiment, the antibody is conjugated to a detectable molecule. In another particular embodiment, the detectable molecule is an enzyme, fluorescent molecule, luminescent molecule, or radioactive molecule.

In a fourteenth aspect, provided herein is a method for inhibiting KIT activity in a cell expressing KIT comprising contacting the cell with an effective amount of an isolated antibody described herein.

In a fifteenth aspect, provided herein is a method for inducing or enhancing apoptosis in a cell expressing KIT comprising contacting the cell with an effective amount of an isolated antibody described herein.

In a sixteenth aspect, provided herein is a method for inducing cell differentiation comprising contacting a cell expressing KIT with an effective amount of an isolated antibody described herein. In one embodiment, the cell is a stem cell.

### 3.1 Terminology

Unless defined otherwise, all technical and scientific terms used herein have the same meaning as is commonly understood by one of ordinary skill in the art.

As used herein and unless otherwise specified, the terms “about” or “approximately” mean within 20%, within 10%, or within 5% (or 1% or less) of a given value or range.

As used herein and unless otherwise specified, “administer” or “administration” refers to the act of injecting or otherwise physically delivering a substance (e.g., an anti-KIT antibody provided herein) into a subject or a patient, such as by mucosal, topical, intradermal, intravenous, intramuscular delivery and/or any other method of physical delivery described herein or known in the art.

As used herein and unless otherwise specified, the terms “antibody” and “immunoglobulin” and “Ig” can be used interchangeably herein and refer to a molecule with an antigen binding site that immunospecifically binds an antigen. Antibodies include, but are not limited to, monoclonal antibodies, polyclonal antibodies, recombinantly produced antibodies, multispecific antibodies (including bi-specific antibodies), human antibodies, humanized antibodies, chimeric antibodies, synthetic antibodies, tetrameric antibodies comprising two heavy chain and two light chain molecule, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single-chain Fvs (scFv) (e.g., including monospecific, bispecific, etc.), camelized antibodies, Fab fragments, F(ab′) fragments, disulfide-linked Fvs (sdFv), anti-idiotypic (anti-Id) antibodies (including, e.g., anti-anti-Id antibodies), and epitope-binding fragments of any of the above. Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof.

As used herein and unless otherwise specified, an “antigen” is a moiety or molecule that contains an epitope, and, as such, also specifically binds to an antibody. In a specific embodiment, the antigen, to which an antibody described herein binds, is KIT (e.g., human KIT), or a fragment thereof, for example, an extracellular domain of KIT (e.g., human KIT) or a D4/D5 region of KIT (e.g., human KIT).

As used herein and unless otherwise specified, an “epitope” is a term in the art and refers to a localized region of an antigen to which an antibody can specifically bind. A region or a polypeptide contributing to an epitope can be contiguous amino acids of the polypeptide or an epitope can come together from two or more non-contiguous regions of the polypeptide.

As used herein and unless otherwise specified, the terms “antigen binding domain,” “antigen binding region,” “antigen binding fragment,” and similar terms refer to a portion of an antibody molecule which comprises the amino acid residues that interact with an antigen and confer on the antibody molecule its specificity for the antigen (e.g., the complementarity determining regions (CDR)). The antigen binding region can be derived from any animal species, such as rodents (e.g., mouse, rat or hamster) and humans. The CDRs of an antibody molecule can be determined by any method well known to one of skill in the art. In particular, the CDRs can be determined according to the Kabat numbering system (see Kabat et al. (1991) Sequences of proteins of immunological interest. (U.S. Department of Health and Human Services, Washington, D.C.) 5th ed.).

As used herein and unless otherwise specified, a “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

As used herein and unless otherwise specified, a “conformational epitope” or “non-linear epitope” or “discontinuous epitope” refers to one comprised of at least two amino acids which are not consecutive amino acids in a single protein chain. For example, a conformational epitope can be comprised of two or more amino acids which are separated by a stretch of intervening amino acids but which are close enough to be recognized by an antibody (e.g., an anti-KIT antibody) described herein as a single epitope. As a further example, amino acids which are separated by intervening amino acids on a single protein chain, or amino acids which exist on separate protein chains, can be brought into proximity due to the conformational shape of a protein structure or complex to become a conformational epitope which can be bound by an anti-KIT antibody described herein. It will be appreciated by one of skill in the art that, in general, a linear epitope bound by an anti-KIT antibody described herein may or may not be dependent on the secondary, tertiary, or quaternary structure of the KIT receptor. For example, in some embodiments, an anti-KIT antibody described herein binds to a group of amino acids regardless of whether they are folded in a natural three dimensional protein structure. In other embodiments, an anti-KIT antibody described herein does not recognize the individual amino acid residues making up the epitope, and require a particular conformation (bend, twist, turn or fold) in order to recognize and bind the epitope.

As used herein and unless otherwise specified, the term “constant region” or “constant domain” refers to an antibody portion, e.g., a carboxyl terminal portion of a light and/or heavy chain which is not directly involved in binding of an antibody to antigen but which exhibits various effector functions, such as interaction with the Fc receptor. The terms refer to a portion of an immunoglobulin molecule having a generally more conserved amino acid sequence relative to an immunoglobulin variable domain.

As used herein and unless otherwise specified, the terms “D4/D5 region” or “D4/D5 domain” refer to a region within a KIT polypeptide spanning the fourth Ig-like extracellular (“D4”) domain, the fifth Ig-like extracellular (“D5”) domain, and the hinge region in between the D4 and D5 domains (“D4-D5 hinge region”), of KIT, in the following order from the amino terminus to the carboxyl terminus: D4, D4-D5 hinge region, and D5. As used herein, amino acids V308 to H515, of FIG. 1 and the polypeptide depicted at FIG. 2A herein are considered the D4/D5 regions or domains.

As used herein and unless otherwise specified, the terms “KIT” or “KIT receptor” or “KIT polypeptide” refer to any form of full-length KIT including, but not limited to, native KIT, an isoform of KIT, an interspecies KIT homolog, or a KIT variant, e.g., naturally occurring (for example, allelic or splice variant, or mutant, e.g., somatic mutant) or artificially constructed variant (for example, a recombinant or chemically modified variant). KIT is a type III receptor tyrosine kinase encoded by the c-kit gene (see, e.g., Yarden et al., Nature, 1986, 323:226-232; Ullrich and Schlessinger, Cell, 1990, 61:203-212; Clifford et al., J. Biol. Chem., 2003, 278:31461-31464; Yarden et al., EMBO J., 1987, 6:3341-3351; Mol et al., J. Biol. Chem., 2003, 278:31461-31464). GenBank™ accession number NM_{—}000222 provides an exemplary human KIT nucleic acid sequence. GenBank™ accession numbers NP_{—}001087241, P10721, and AAC50969 provide exemplary human KIT amino acid sequences. GenBank™ accession number AAH75716 provides an exemplary murine KIT amino acid sequence. Native KIT comprises five extracellular immunoglobulin (Ig)-like domains (D1, D2, D3, D4, D5), a single transmembrane region, an inhibitory cytoplasmic juxtamembrane domain, and a split cytoplasmic kinase domain separated by a kinase insert segment (see, e.g., Yarden et al., Nature, 1986, 323:226-232; Ullrich and Schlessinger, Cell, 1990, 61:203-212; Clifford et al., J. Biol. Chem., 2003, 278:31461-31464). An exemplary amino acid sequence of the D4/D5 region of human KIT is provided in FIG. 1, at amino acid residues V308 to H515. In a specific embodiment, KIT is human KIT. In a particular embodiment, KIT can exist as a monomer, dimer, multimer, native form, or denatured form.

As used herein and unless otherwise specified, the terms “effective amount” or “therapeutically effective amount” refer to an amount of a therapy (e.g., an antibody or pharmaceutical composition provided herein) which is sufficient to reduce and/or ameliorate the severity and/or duration of a given disease and/or a symptom related thereto. These terms also encompass an amount necessary for the reduction or amelioration of the advancement or progression of a given disease, reduction or amelioration of the recurrence, development or onset of a given disease, and/or to improve or enhance the prophylactic or therapeutic effect(s) of another therapy (e.g., a therapy other than an anti-KIT antibody provided herein). In some embodiments, “effective amount” as used herein also refers to the amount of an antibody described herein to achieve a specified result (e.g., inhibition of a KIT biological activity of a cell, such as inhibition of cell proliferation or cell survival, or enhancement or induction of apoptosis or cell differentiation).

In the context of a peptide or a polypeptide, the term “fragment” as used herein refers to a peptide or polypeptide that comprises less than full length amino acid sequence. Such a fragment can arise, for example, from a truncation at the amino terminus, a truncation at the carboxy terminus, and/or an internal deletion of a residue(s) from the amino acid sequence. Fragments can, for example, result from alternative RNA splicing or from in vivo protease activity. In certain embodiments, KIT fragments or antibody fragments (e.g., antibody fragments that immunospecifically bind to a KIT polypeptide) include polypeptides comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a KIT polypeptide or an antibody (e.g., antibody that immunospecifically bind to a KIT polypeptide), respectively. In a specific embodiment, a fragment of a KIT polypeptide or an antibody (e.g., antibody that immunospecifically bind to a KIT polypeptide) retains at least 1, at least 2, or at least 3 functions of the polypeptide or antibody.

As used herein and unless otherwise specified, the term “heavy chain” when used in reference to an antibody refers to any distinct types, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG1, IgG2, IgG3 and IgG4. In a specific embodiment, the heavy chain is a human heavy chain.

As used herein and unless otherwise specified, the term “host cell” as used herein refers to a particular cell transfected with a nucleic acid molecule and the progeny or potential progeny of such a cell. Progeny of such a cell may not be identical to the parent cell transfected with the nucleic acid molecule due to mutations or environmental influences that can occur in succeeding generations or integration of the nucleic acid molecule into the host cell genome.

As used herein and unless otherwise specified, the term “human antibodies” refers to an antibody that comprises a human variable region and, generally, a human constant region.

As used herein and unless otherwise specified, the terms “immunospecifically binds,” “immunospecifically recognizes,” “specifically binds,” and “specifically recognizes” are analogous terms in the context of antibodies and refer to molecules that specifically bind to an antigen (e.g., epitope or immune complex) as understood by one skilled in the art. For example, a molecule that specifically binds to an antigen can bind to other peptides or polypeptides, generally with lower affinity as determined by, e.g., immunoassays, Biacore™, KinExA 3000 instrument (Sapidyne Instruments, Boise, Id.), or other assays known in the art. In a specific embodiment, molecules that immunospecifically bind to an antigen bind to the antigen with a Kₐ that is at least 2 logs, 2.5 logs, 3 logs, 4 logs or greater than the Kₐ when the molecules bind to another antigen. In another specific embodiment, molecules that immunospecifically bind to an antigen do not cross react with other proteins. In another specific embodiment, molecules that immunospecifically bind to an antigen do not cross react with other non-KIT proteins.

As used herein and unless otherwise specified, the term “in combination” in the context of the administration of other therapies refers to the use of more than one therapy. The use of the term “in combination” does not restrict the order in which therapies are administered. The therapies may be administered serially (or sequentially) or concurrently.

As used herein and unless otherwise specified, an “isolated” or “purified” antibody is substantially free of cellular material or other contaminating proteins from the cell or tissue source from which the antibody is derived, or substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, for example, different post-translational modified forms of an antibody or other different versions of an antibody (e.g., antibody fragments). When the antibody is recombinantly produced, it is also generally substantially free of culture medium, i.e., culture medium represents less than about 20%, 10%, 5%, 2%, 1%, 0.5%, or 0.1% of the volume of the protein preparation. When the antibody is produced by chemical synthesis, it is generally substantially free of chemical precursors or other chemicals, i.e., it is separated from chemical precursors or other chemicals which are involved in the synthesis of the protein. Accordingly such preparations of the antibody have less than about 30%, 20%, 10%, 5% (by dry weight) of chemical precursors or compounds other than the antibody of interest. In a specific embodiment, antibodies described herein are isolated or purified.

As used herein and unless otherwise specified, an “isolated” polynucleotide or nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, a nucleic acid molecule(s) encoding an antibody described herein is isolated or purified.

The terms “Kabat numbering,” and like terms are recognized in the art and refer to a system of numbering amino acid residues in the heavy and light chain variable regions of an antibody, or an antigen binding portion thereof (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35 (“CDR1”), amino acid positions 50 to 65 (“CDR2”), and amino acid positions 95 to 102 (“CDR3”). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

As used herein and unless otherwise specified, the terms “KIT-mediated disorder” or “KIT-mediated disease” are used interchangeably and refer to any disease that is completely or partially caused by or is the result of KIT (e.g., KIT expression and/or activity). In certain embodiments, KIT is aberrantly (e.g., highly) expressed on the surface of a cell. In particular embodiments, KIT expression is at least about 10%, 20%, 30%, 40%, 50%, 60%, 70%, 80%, 90%, or 100% higher than KIT expression on the surface of a control cell (e.g., a cell expressing normal levels of KIT, for example, a mast cell, stem cell, brain cell, melanoblast, or ovary cell). In some embodiments, KIT can be aberrantly upregulated on a particular cell type. In particular embodiments, KIT signaling can be aberrantly upregulated in a particular cell type. In particular embodiments, KIT signaling is at least about 10%, 20%, 30%, 40%, 50%, 60%, 70%, 80%, 90%, or 100% higher than KIT KIT signaling of a control cell (e.g., a cell cell containg normal KIT signaling, for example, a mast cell, stem cell, brain cell, melanoblast, or ovary cell). In certain certain embodiments, normal, aberrant or excessive cell signaling is caused by binding of KIT to a KIT ligand. In other embodiments, aberrant or excessive cell signaling occurs independent of binding of KIT to a KIT ligand. In certain embodiments, a KIT-mediated disease is fibrosis or an inflammatory disorder, e.g., inflammatory bowel disease (IBD), such as Crohn's disease (CD) or ulcerative colitis (UC). In other embodiments, a KIT-mediated disease is cancer, such as lung cancer, leukemia, neuroblastoma, or gastrointestinal stromal tumor (GIST).

As used herein and unless otherwise specified, the term “light chain” when used in reference to an antibody refers to any distinct types, e.g., kappa (κ) of lambda (λ) based on the amino acid sequence of the constant domains. Light chain amino acid sequences are well known in the art. In specific embodiments, the light chain is a human light chain.

As used herein and unless otherwise specified, the terms “manage,” “managing,” and “management” refer to the beneficial effects that a subject derives from a therapy (e.g., a prophylactic or therapeutic agent), which does not result in a cure of a KIT-mediated disease or disorder. In certain embodiments, a subject is administered one or more therapies (e.g., prophylactic or therapeutic agents, such as an antibody described herein) to “manage” a KIT-mediated disease (e.g., cancer, inflammatory condition, or fibrosis), one or more symptoms thereof, so as to prevent the progression or worsening of the disease.

As used herein and unless otherwise specified, the term “monoclonal antibody” refers to an antibody obtained from a population of homogenous or substantially homogeneous antibodies, and each monoclonal antibody will typically recognize a single epitope on the antigen. In specific embodiments, a “monoclonal antibody,” as used herein, is an antibody produced by a single hybridoma or other cell (e.g., host cell producing a recombinant antibody), wherein the antibody immunospecifically binds to a KIT epitope (e.g., an epitope of a D4/D5 region of human KIT) as determined, e.g., by ELISA or other antigen-binding or competitive binding assay known in the art or in the Examples provided herein. Generally, a population of monoclonal antibodies can be generated by cells, a population of cells, or a cell line. The term “monoclonal” is not limited to any particular method for making the antibody. For example, monoclonal antibodies described herein can be made by the hybridoma method as described in Kohler et al.; Nature, 256:495 (1975) or can be isolated from phage libraries using the techniques as described herein, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel et al., eds., John Wiley and Sons, New York).

As used herein and unless otherwise specified, the term “naturally occurring” or “native” when used in connection with biological materials such as nucleic acid molecules, polypeptides, host cells, and the like, refers to those which are found in nature and not manipulated by a human being. Likewise, the term “non-naturally occurring” when used in the context of biological materials such as antibodies, nucleic acid molecules, polypeptides, host cells, and the like, refers to those which are not found in nature and/or are manipulated by a human being.

As used herein and unless otherwise specified, the term “pharmaceutically acceptable” as used herein means being approved by a regulatory agency of the Federal or a state government, or listed in the U.S. Pharmacopeia, European Pharmacopeia or other generally recognized Pharmacopeia for use in animals, and more particularly in humans.

As used herein and unless otherwise specified, the term “polyclonal antibodies” refers to an antibody population generated in an immunogenic response to a protein having many epitopes and thus includes a variety of different antibodies directed to the same and to different epitopes within the protein. Methods for producing polyclonal antibodies are known in the art (See, e.g., see, for example, Chapter 11 in: Short Protocols in Molecular Biology, (2002) 5th Ed., Ausubel et al., eds., John Wiley and Sons, New York).

As used herein and unless otherwise specified, the term “polynucleotide,” “nucleotide,” nucleic acid” “nucleic acid molecule” and other similar terms are used interchangeable and include DNA, RNA, mRNA and the like.

As used herein and unless otherwise specified, the terms “impede” or “impeding” in the context of a KIT-mediated disorder or disease refer to the total or partial inhibition (e.g., less than 100%, 95%, 90%, 80%, 70%, 60%, 50%, 40%, 30%, 20%, 10%, or 5%) or blockage of the development, recurrence, onset or spread of a KIT-mediated disease and/or symptom related thereto, resulting from the administration of a therapy or combination of therapies provided herein (e.g., a combination of prophylactic or therapeutic agents, such as an antibody described herein).

As used herein and unless otherwise specified, the term “prophylactic agent” refers to any agent that can totally or partially inhibit the development, recurrence, onset or spread of a KIT-mediated disease and/or symptom related thereto in a subject. In certain embodiments, the term “prophylactic agent” refers to an antibody described herein. In certain other embodiments, the term “prophylactic agent” refers to an agent other than an antibody described herein. Generally, a prophylactic agent is an agent which is known to be useful to or has been or is currently being used to prevent a KIT-mediated disease and/or a symptom related thereto or impede the onset, development, progression and/or severity of a KIT-mediated disease and/or a symptom related thereto. In specific embodiments, the prophylactic agent is a human anti-KIT antibody, such as a fully human anti-KIT monoclonal antibody.

As used herein and unless otherwise specified, the term “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse, rabbit, goat, or cow) that is transgenic and/or transchromosomal for human immunoglobulin genes (see e.g., Taylor, L. D. et al. (1992) Nucl. Acids Res. 20:6287-6295) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, can not naturally exist within the human antibody germline repertoire in vivo.

As used herein and unless otherwise specified, the term “serum titer” refers to an average serum titer in a population of least 10, preferably at least 20, and most preferably at least 40 subjects (e.g., humans) up to about 100, 1000 or more.

As used herein and unless otherwise specified, the term “side effects” encompasses unwanted and adverse effects of a therapy (e.g., a prophylactic or therapeutic agent). Unwanted effects are not necessarily adverse. An adverse effect from a therapy (e.g., a prophylactic or therapeutic agent) can be harmful or uncomfortable or risky. Examples of side effects include, diarrhea, cough, gastroenteritis, wheezing, nausea, vomiting, anorexia, abdominal cramping, fever, pain, loss of body weight, dehydration, alopecia, dyspenea, insomnia, dizziness, mucositis, nerve and muscle effects, fatigue, dry mouth, and loss of appetite, rashes or swellings at the site of administration, flu-like symptoms such as fever, chills and fatigue, digestive tract problems and allergic reactions. Additional undesired effects experienced by patients are numerous and known in the art. Many are described in the Physician's Desk Reference (63^{rd} ed., 2009).

As used herein and unless otherwise specified, the terms “subject” and “patient” are used interchangeably. As used herein, a subject is preferably a mammal such as a non-primate (e.g., cows, pigs, horses, cats, dogs, goats, rabbits, rats, mice, etc.) or a primate (e.g., monkey and human), most preferably a human. In one embodiment, the subject is a mammal, preferably a human, having a KIT-mediated disorder or disease. In another embodiment, the subject is a mammal, preferably a human, at risk of developing a KIT-mediated disorder or disease.

As used herein and unless otherwise specified, the term “synergistic,” or “synergy” refers to the effect of the administration of an anti-KIT antibody or pharmaceutical composition thereof in combination with one or more additional therapies (e.g., agents or surgery), which combination is more effective than the additive effects of any two or more single therapies (e.g., agents or surgery). In a specific embodiment, a synergistic effect of a combination therapy permits the use of lower dosages (e.g., sub-optimal doses) of an anti-KIT antibody described herein or an additional therapy and/or less frequent administration of an anti-KIT antibody described herein or an additional therapy to a subject. In certain embodiments, the ability to utilize lower dosages of an anti-KIT antibody or of an additional therapy and/or to administer an anti-KIT antibody or said additional therapy less frequently reduces the toxicity associated with the administration of an anti-KIT antibody or of said additional therapy, respectively, to a subject without reducing the efficacy of an anti-KIT antibody or of said additional therapy, respectively, in the treatment of a KIT-mediated disorder or disease. In some embodiments, a synergistic effect results in improved efficacy of an anti-KIT antibody described herein and of each of said additional therapies in treating a KIT-mediated disorder or disease. In some embodiments, a synergistic effect of a combination of an anti-KIT antibody described herein and one or more additional therapies avoids or reduces adverse or unwanted side effects associated with the use of any single therapy.

As used herein and unless otherwise specified, the terms “therapies” and “therapy” can refer to any protocol(s), method(s), compositions, formulations, and/or agent(s) that can be used in the prevention, treatment, management, or amelioration of a condition or disorder or symptom thereof (e.g., cancer or one or more symptoms or condition associated therewith; inflammatory condition or one or more symptoms or condition associated therewith; fibrosis or one or more symptoms or condition associated therewith). In certain embodiments, the terms “therapies” and “therapy” refer to drug therapy, adjuvant therapy, radiation, surgery, biological therapy, supportive therapy, and/or other therapies useful in treatment, management, prevention, or amelioration of a condition or disorder or one or more symptoms thereof (e.g., cancer or one or more symptoms or condition associated therewith; inflammatory condition or one or more symptoms or condition associated therewith; fibrosis or one or more symptoms or condition associated therewith). In certain embodiments, the term “therapy” refers to a therapy other than an anti-KIT antibody described herein or pharmaceutical composition thereof. In specific embodiments, an “additional therapy” and “additional therapies” refer to a therapy other than a treatment using an anti-KIT antibody described herein or pharmaceutical composition. In a specific embodiment, a therapy includes the use of an anti-KIT antibody described herein as an adjuvant therapy. For example, using an anti-KIT antibody described herein in conjunction with a drug therapy, biological therapy, surgery, and/or supportive therapy.

As used herein and unless otherwise specified, the term “therapeutic agent” refers to any agent that can be used in the treatment, management or amelioration of a KIT-mediated disease and/or a symptom related thereto. In certain embodiments, the term “therapeutic agent” refers to an antibody described herein. In certain other embodiments, the term “therapeutic agent” refers to an agent other than an antibody described herein. Preferably, a therapeutic agent is an agent which is known to be useful for, or has been or is currently being used for the treatment, management or amelioration of a KIT-mediated disease or one or more symptoms related thereto. In specific embodiments, the therapeutic agent is a human anti-KIT antibody, such as a fully human anti-KIT monoclonal antibody.

As used herein and unless otherwise specified, the terms “treat,” “treatment” and “treating” refer to the reduction or amelioration of the progression, severity, and/or duration of a KIT-mediated disease (e.g., cancer, inflammatory disorder, or fibrosis) resulting from the administration of one or more therapies (including, but not limited to, the administration of one or more prophylactic or therapeutic agents, such as an antibody provided herein).

As used herein and unless otherwise specified, the terms “variable region” or “variable domain” refer to a portion of an antibody, generally, a portion of a light or heavy chain, typically about the amino-terminal 120 to 130 amino acids in the heavy chain and about 100 to 110 amino acids in the light chain, which differ extensively in sequence among antibodies and are used in the binding and specificity of a particular antibody for its particular antigen. The variability in sequence is concentrated in those regions called complimentarity determining regions (CDRs) while the more highly conserved regions in the variable domain are called framework regions (FR). The CDRs of the light and heavy chains are primarily responsible for the interaction of the antibody with antigen. Numbering of amino acid positions used herein is according to the EU Index, as in Kabat et al. (1991) Sequences of Proteins of Immunological Interest, Fifth Edition, U.S. Department of Health and Human Services, NIH Publication No. 91-3242 (“Kabat et al.”). In certain embodiments, the variable region is a human variable region.

### 4. BRIEF DESCRIPTION OF THE FIGURES

FIG. 1 depicts the amino acid sequence of full length human KIT (SEQ ID NO: 1), GenBank™ accession number AAC50969. The first through fifth extracellular Ig-like domains (i.e., D1, D2, D3, D4, and D5) are indicated; “{” depicts the amino-terminal residue of each domain and “}” depicts the carboxyl-terminal residue of each domain. The D1 domain is depicted at P34 to R112, the D2 domain is depicted at D113 to P206, the D3 domain is depicted at A207 to D309, the D4 domain is depicted at K310 to N410, the hinge region between D4 and D5 is depicted at V409 to N410, and the D5 domain is depicted at T411 to K509. Also, the D1/D2 hinge region is depicted at D113 to L117; the D2/D3 hinge region is depicted at P206 to A210; and the D3/D4 hinge region is depicted at D309 to G311. The D4/D5 region comprises K310 to K509. The transmembrane domain comprises residues F525 to Q545, and the kinase domain comprises residues K589 to S933.

FIG. 2A depicts the amino acid sequence of the recombinant KIT D4/D5 polypeptide used to generate antibodies. Human KIT amino acids V308 to H515 (SEQ ID NO: 171) are depicted in bold. The recombinant KIT D4/D5 polypeptide depicted (SEQ ID NO: 170) contains (i) the first 33 amino acids (i.e., M1 to E33) of the amino terminus of human KIT (including the signal peptide (underlined, not bold) which is cleaved during processing and, as such, was not utilized as part of the antigen used to generate antibodies), (ii) the D4/D5 region of human KIT (bold), and (iii) a 5×His tag (italics) at the carboxyl terminus.

FIG. 2B depicts the amino acid sequence of the recombinant D4 domain polypeptide used in binding assays (see section 6). In particular, the recombinant D4 domain polypeptide comprising residues V308 to K412 (SEQ ID NO: 173), depicted as bold, of human KIT—includes the D4 domain (residues K310 to N410). The recombinant D4 domain polypeptide amino acid sequence depicted (SEQ ID NO: 172) contains (i) the first 33 amino acids of the amino terminus of human KIT (including the signal peptide (underlined, not bold) which is cleaved during processing and, as such, was not utilized as part of the polypeptide used in binding assays), (ii) the D4 domain (bold, single-underlined) of human KIT, and (iii) a SxHis tag (italics) at the carboxyl terminus.

FIG. 2C depicts the amino acid sequence of the recombinant D5 domain polypeptide used in binding assays (see section 6). In particular, the recombinant D5 domain polypeptide—comprising residues N410 to H515 (SEQ ID NO: 175), depicted as bold, of human KIT—includes the D5 region (residues T411 to K509) of human KIT. The recombinant D5 domain polypeptide amino acid sequence depicted (SEQ ID NO: 174) contains (i) the first 33 amino acids of the amino terminus of human KIT (including the signal peptide (underlined, not bold) which is cleaved during processing and, as such, was not utilized as part of the polypeptide used in binding assays), (ii) the D5 domain (bold, double-underlined) of human KIT, and (iii) a SxHis tag (italics) at the carboxyl terminus.

FIGS. 3A-B depict the variable light chain region amino acid sequence of anti-KIT antibodies Ab1 (SEQ ID NO: 176), Ab2 (SEQ ID NO: 177), Ab3 (SEQ ID NO: 178), Ab4 (SEQ ID NO: 179), Ab5 (SEQ ID NO: 180), Ab6 (SEQ ID NO: 181), Ab7 (SEQ ID NO: 182), Ab8 (SEQ ID NO: 183), Ab9 (SEQ ID NO: 184), Ab10 (SEQ ID NO: 185), Ab11 (SEQ ID NO: 186), and Ab12 (SEQ ID NO: 187). The framework regions (FR1, FR2, FR3, and FR4), and CDRs (CDR1, CDR2, and CDR3) are indicated.

FIGS. 4A-B depict the variable heavy chain region amino acid sequence of anti-KIT antibodies Ab1 (SEQ ID NO: 188), Ab2 (SEQ ID NO: 189), Ab3 (SEQ ID NO: 190), Ab4 (SEQ ID NO: 191), Ab5 (SEQ ID NO: 192), Ab6 (SEQ ID NO: 193), Ab7 (SEQ ID NO: 194), Ab8 (SEQ ID NO: 195), Ab9 (SEQ ID NO: 196), Ab10 (SEQ ID NO: 197), Ab11 (SEQ ID NO: 198), and Ab12 (SEQ ID NO: 199). The framework regions (FR1, FR2, FR3, and FR4), and CDRs (CDR1, CDR2, and CDR3) are indicated.

FIG. 5A depicts the amino acid sequences of the VL domain (SEQ ID NO: 176, 350, or 963) and VH domain (SEQ ID NO: 188) of anti-KIT antibody Ab1. The CDRs (CDR1, CDR2, and CDR3) are bold and underlined.

FIG. 5B depicts the amino acid sequences of the VL domain (SEQ ID NO: 177) and VH domain (SEQ ID NO: 189) of anti-KIT antibody Ab2. The CDRs (CDR1, CDR2, and CDR3) are bold and underlined.

FIG. 5C depicts the amino acid sequences of the VL domain (SEQ ID NO: 178) and VH domain (SEQ ID NO: 190) of anti-KIT antibody Ab3. The CDRs (CDR1, CDR2, and CDR3) are bold and underlined.

FIG. 5D depicts the amino acid sequences of the VL domain (SEQ ID NO: 179) and VH domain (SEQ ID NO: 191) of anti-KIT antibody Ab4. The CDRs (CDR1, CDR2, and CDR3) are bold and underlined.

FIG. 5E depicts the amino acid sequences of the VL domain (SEQ ID NO: 180) and VH domain (SEQ ID NO: 192) of anti-KIT antibody Ab5. The CDRs (CDR1, CDR2, and CDR3) are bold and underlined.

FIG. 5F depicts the amino acid sequences of the VL domain (SEQ ID NO: 181) and VH domain (SEQ ID NO: 193) of anti-KIT antibody Ab6. The CDRs (CDR1, CDR2, and CDR3) are bold and underlined.

FIG. 5G depicts the amino acid sequences of the VL domain (SEQ ID NO: 182) and VH domain (SEQ ID NO: 194) of anti-KIT antibody Ab7. The CDRs (CDR1, CDR2, and CDR3) are bold and underlined.

FIG. 5H depicts the amino acid sequences of the VL domain (SEQ ID NO: 183) and VH domain (SEQ ID NO: 195) of anti-KIT antibody Ab8. The CDRs (CDR1, CDR2, and CDR3) are bold and underlined.

FIG. 5I depicts the amino acid sequences of the VL domain (SEQ ID NO: 184) and VH domain (SEQ ID NO: 196) of anti-KIT antibody Ab9. The CDRs (CDR1, CDR2, and CDR3) are bold and underlined.

FIG. 5J depicts the amino acid sequences of the VL domain (SEQ ID NO: 185) and VH domain (SEQ ID NO: 197) of anti-KIT antibody Ab10. The CDRs (CDR1, CDR2, and CDR3) are bold and underlined.

FIG. 5K depicts the amino acid sequences of the VL domain (SEQ ID NO: 186) and VH domain (SEQ ID NO: 198) of anti-KIT antibody Ab11. The CDRs (CDR1, CDR2, and CDR3) are bold and underlined.

FIG. 5L depicts the amino acid sequences of the VL domain (SEQ ID NO: 187) and VH domain (SEQ ID NO: 199) of anti-KIT antibody Ab12. The CDRs (CDR1, CDR2, and CDR3) are bold and underlined.

FIG. 5M depicts the amino acid sequences of the VL domain (SEQ ID NO: 312) and VH domain (SEQ ID NO: 320) of anti-KIT antibody Ab13. The CDRs (CDR1, CDR2, and CDR3) are bold and underlined.

FIG. 5N depicts the amino acid sequences of the VL domain (SEQ ID NO: 313) and VH domain (SEQ ID NO: 321) of anti-KIT antibody Ab14. The CDRs (CDR1, CDR2, and CDR3) are bold and underlined.

FIG. 5O depicts the amino acid sequences of the VL domain (SEQ ID NO: 314) and VH domain (SEQ ID NO: 322) of anti-KIT antibody Ab15. The CDRs (CDR1, CDR2, and CDR3) are bold and underlined.

FIG. 5P depicts the amino acid sequences of the VL domain (SEQ ID NO: 315) and VH domain (SEQ ID NO: 323) of anti-KIT antibody Ab16. The CDRs (CDR1, CDR2, and CDR3) are bold and underlined.

FIG. 5Q depicts the amino acid sequences of the VL domain (SEQ ID NO: 316) and VH domain (SEQ ID NO: 324) of anti-KIT antibody Ab17. The CDRs (CDR1, CDR2, and CDR3) are bold and underlined.

FIG. 5R depicts the amino acid sequences of the VL domain (SEQ ID NO: 317) and VH domain (SEQ ID NO: 325) of anti-KIT antibody Ab18. The CDRs (CDR1, CDR2, and CDR3) are bold and underlined.

FIG. 5S depicts the amino acid sequences of the VL domain (SEQ ID NO: 318) and VH domain (SEQ ID NO: 326) of anti-KIT antibody Ab19. The CDRs (CDR1, CDR2, and CDR3) are bold and underlined.

FIG. 5T depicts the amino acid sequences of the VL domain (SEQ ID NO: 319) and VH domain (SEQ ID NO: 327) of anti-KIT antibody Ab20. The CDRs (CDR1, CDR2, and CDR3) are bold and underlined.

FIG. 5U depicts the amino acid sequences of the VL domain (SEQ ID NO: 328 or 964) and VH domain (SEQ ID NO: 329) of anti-KIT antibody Ab21. The CDRs (CDR1, CDR2, and CDR3) are bold and underlined. Also depicted are the amino acid sequences of the light chain constant domain (SEQ ID NO: 344 or 965) and of the heavy chain constant domain (SEQ ID NO: 345) of antibody Ab21.

FIG. 5V depicts the amino acid sequences of the VL domain (SEQ ID NO: 1162) and VH domain (SEQ ID NO: 1163) of anti-KIT antibody Ab167. The CDRs (CDR1, CDR2, and CDR3) are bold and underlined.

FIG. 5W depicts the amino acid sequences of the VL domain (SEQ ID NO: 1164) and VH domain (SEQ ID NO: 1165) of anti-KIT antibody Ab63. The CDRs (CDR1, CDR2, and CDR3) are bold and underlined.

FIG. 6 depicts the binding activity of full-length human IgG1 antibodies Ab1 and Ab21 to the D4/D5 region of human KIT by solid phase ELISA.

FIG. 7A-B depict the inhibition of phosphorylation of the cytoplasmic domain of human KIT by antibodies Ab1 (FIG. 7A) and Ab21 (FIG. 7B) by cell based phosphorylation assays. The 50% inhibition concentration of phosphorylation for Ab1 and Ab21 are 2.2 nM and 1.3 nM, respectively.

FIG. 8 depicts a Western blot showing inhibition of KIT phosphorylation by Ab21 in the small cell lung cancer cell line H526. Detection of phosphorylated KIT (“pKIT”), KIT, beta-tubulin, phosphorylated Erk-1,2 (“pErk-1,2”), and total Erk-1,2 are labeled accordingly.

FIG. 9 depicts a Western blot showing inhibition of KIT phosphorylation by Ab21 in CHO cells expressing full-length, wild-type human KIT (CHO/KIT WT). “TL” indicates total lysate, and detection of pKIT, total KIT, beta-tubulin, pErk-1,2, and total Erk-1,2 are labeled accordingly.

FIG. 10 depicts a bar graft showing that antibody Ab21 can inhibit colony formation by CHO/KIT WT cells in soft agar assays. Cells were treated with or without antibody Ab21 and in the presence or absence of SCF stimulation.

### 5. DETAILED DESCRIPTION

Provided herein are antibodies (e.g., human antibodies), that immunospecifically bind to a KIT polypeptide (e.g., a KIT polypeptide containing the D4/D5 region of human KIT). Also provided are isolated nucleic acids encoding such antibodies (e.g., human antibodies). Further provided are vectors and host cells comprising nucleic acids encoding such antibodies. Also provided are methods of making such antibodies. Also provided herein is a method of treating or managing a KIT-mediated disorder or disease (e.g., cancer, inflammatory condition, or fibrosis) comprising administering one or more antibodies described herein. Also provided herein is a method of diagnosing a KIT-mediated disorder or disease (e.g., cancer, inflammatory condition, or fibrosis) comprising contacting a sample with one or more antibodies described herein. Further provided herein is a method for inhibiting KIT activity in a cell expressing KIT comprising contacting the cell with an effective amount of an antibody or antibodies described herein. Also further provided herein is a method for inducing or enhancing cell differentiation or apoptosis in a cell expressing KIT comprising contacting the cell with an effective amount of an antibody or antibodies described herein.

### 5.1 Antibodies

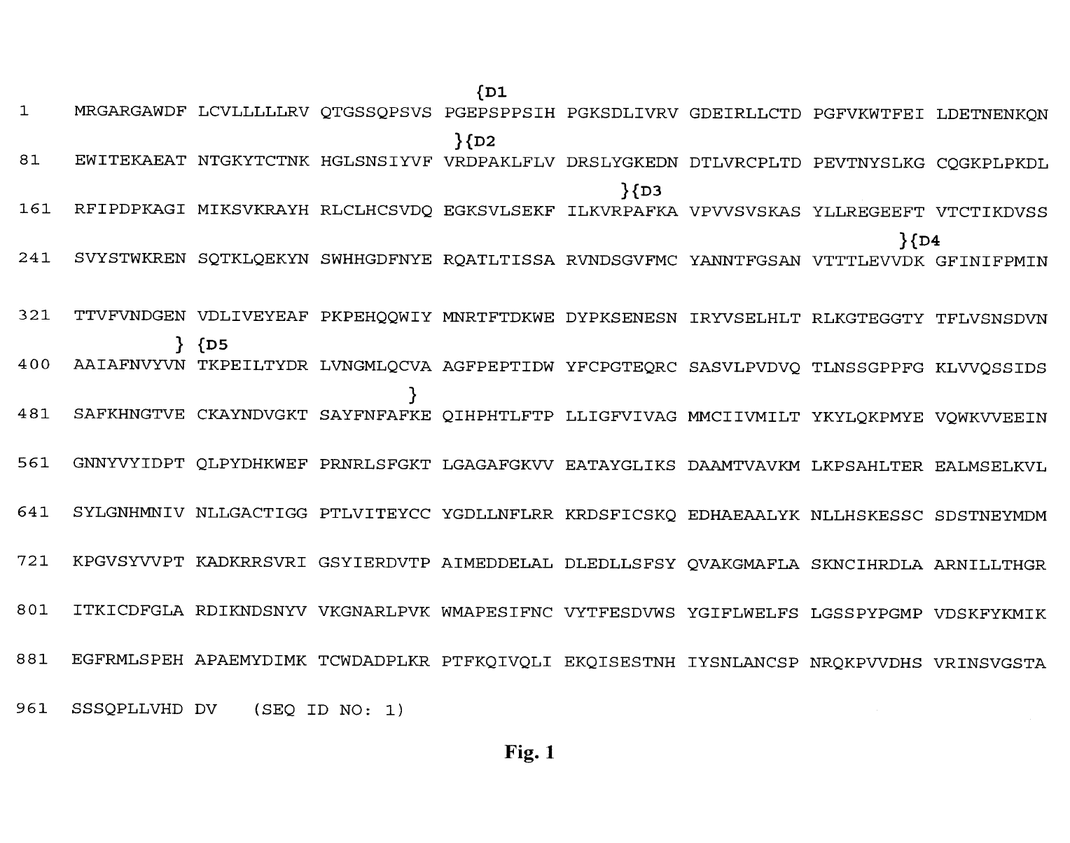

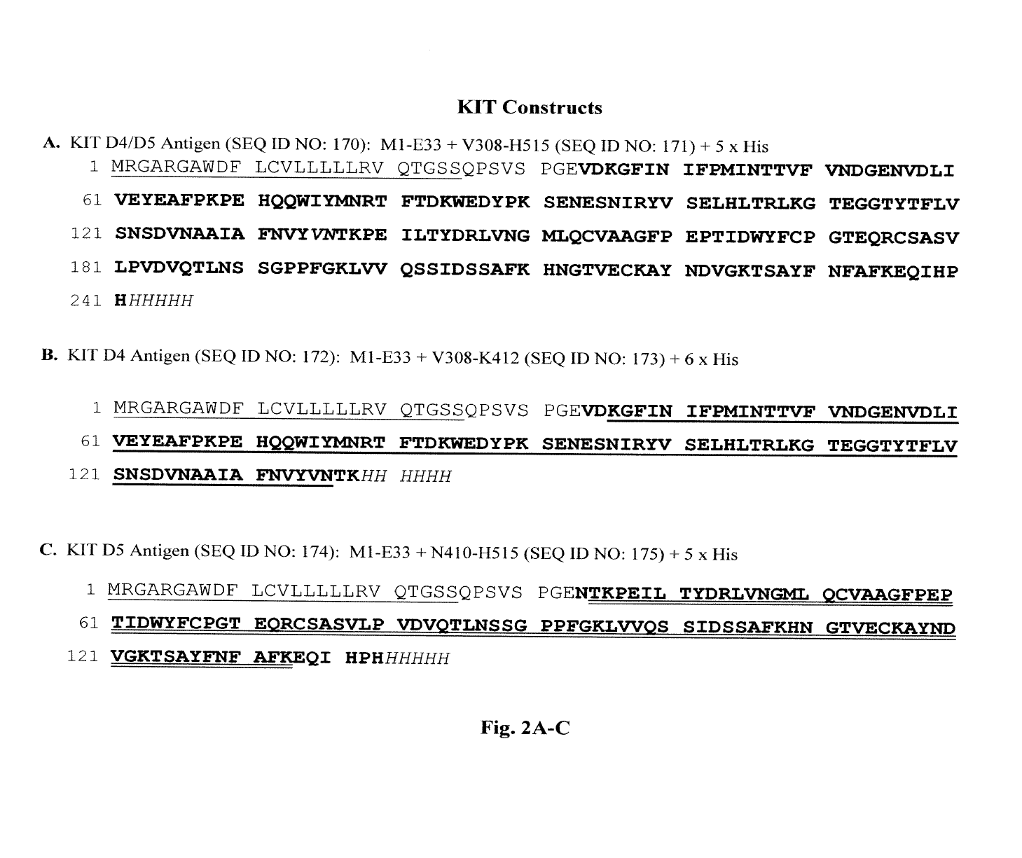

In specific aspects, provided herein are isolated antibodies that immunospecifically bind to the D4/D5 region of KIT, e.g., human KIT. Amino acid residues V308 to H515 (SEQ ID NO: 171) of FIGS. 1 and 2 represent an exemplary D4/D5 region of human KIT. In a specific embodiment, an antibody described herein immunospecifically binds to a D4 domain of KIT, e.g., human KIT, for example, amino acid residues K310 to N410 of human KIT (see FIGS. 1, 2A and 2B). In another specific embodiment, an antibody described herein immunospecifically binds to a D5 domain of KIT, e.g., human KIT, for example, amino acid residues V409 to N410 of human KIT (see FIGS. 1, 2A and 2C). In yet another specific embodiment, an antibody described herein immunospecifically binds to the hinge region between a D4 domain and a D5 domain of KIT, e.g., human KIT. In a specific embodiment, an antibody described herein immunospecifically binds to a D4/D5 region of KIT, e.g., human KIT, but does not bind to a KIT antigen consisting essentially of a D4 domain only or a KIT antigen consisting essentially of a D5 domain only.

In a particular embodiment, an antibody described herein immunospecifically binds to the amino acid sequence of SEQ ID NO: 170 or 171. In a specific embodiment, an antibody described herein immunospecifically binds to a D4 domain of KIT, e.g., human KIT. In a particular embodiment, an antibody described herein immunospecifically binds to the amino acid sequence of SEQ ID NO: 172 or 173. In another specific embodiment, an antibody described herein immunospecifically binds to a D5 domain of KIT, e.g., human KIT. In a particular embodiment, an antibody described herein immunospecifically binds to the amino acid sequence of SEQ ID NO: 174 or 175. In yet another specific embodiment, an antibody described herein immunospecifically binds to a D4-D5 hinge region of KIT, e.g., human KIT.

In particular embodiments, an antibody described herein does not bind the extracellular ligand binding site of KIT, e.g., the SCF binding site of KIT.

In particular embodiments, an antibody described herein does not inhibit ligand binding to KIT, e.g., does not inhibit KIT ligand (e.g., SCF) binding to KIT. In certain embodiments, an antibody described herein does not block or inhibit KIT dimerization. In a particular embodiment, an antibody described herein does not disrupt a KIT dimer (for example, does not induce dissociation of a KIT dimer into KIT monomers). In particular embodiments, an antibody described herein does not inhibit KIT dimerization and/or does not inhibit or block KIT ligand (e.g., SCF) binding to KIT (e.g., human KIT).

In certain embodiments, an antibody described herein blocks or inhibits KIT dimerization or disrupts a KIT dimer (for example, induces dissociation of a KIT dimer into KIT monomers). In particular embodiments, an antibody described herein inhibits KIT dimerization or induces dissociation of a KIT dimer, and does not inhibit or block KIT ligand (e.g., SCF) binding to KIT (e.g., human KIT).

In particular embodiments, an antibody provided herein does not immunospecifically bind to a denatured form of KIT, e.g., human KIT.

In specific aspects, antibodies described herein are inhibitory antibodies, that is, antibodies that inhibit KIT activity, i.e., one or more KIT activities. Non-limiting examples of KIT activities include KIT dimerization, KIT phosphorylation (e.g., tyrosine phosphorylation), signaling downstream of KIT (e.g., Stat, AKT, MAPK, or Ras signaling), induction or enhancement of gene transcription (e.g., c-Myc), induction or enhancement of cell proliferation or cell survival. In a particular embodiment, an antibody described herein inhibits KIT phosphorylation. In a specific embodiment, an antibody described herein inhibits KIT tyrosine phosphorylation in the KIT cytoplasmic domain. In another particular embodiment, an antibody described herein inhibits cell proliferation. In yet another particular embodiment, an antibody described herein inhibits cell survival. In a specific embodiment, an antibody described herein induces apoptosis. In another specific embodiment, an antibody described herein induces cell differentiation, e.g., cell differentiation in a cell expressing KIT, e.g., human KIT. In a particular embodiment, an antibody described herein inhibits KIT activity but does not inhibit KIT dimerization. In another particular embodiment, an antibody described herein inhibits KIT activity and does not inhibit ligand binding to KIT, e.g., does not inhibit KIT ligand (e.g., SCF) binding to KIT, but does inhibit KIT dimerization.

In a specific embodiment, an antibody described herein (i) immunospecifically binds to a KIT polypeptide comprising the D4/D5 region of human KIT, (ii) inhibits KIT phosphorylation (e.g., tyrosine phosphorylation), (iii) does not affect KIT ligand (e.g., SCF) binding to KIT, and (iv) does not inhibit KIT dimerization.

The antibodies provided herein generally do not immunospecifically bind to the D1, D2, or D3 domain of the extracellular domain of KIT, e.g., human KIT. That is, in some embodiments, an antibody described herein does not immunospecifically bind to a D1 domain of the extracellular domain of KIT (e.g., human KIT); in some embodiments, an antibody described herein does not immunospecifically bind to a D2 domain of the extracellular domain of KIT (e.g., human KIT); and in some embodiments, an antibody described herein does not immunospecifically bind to a D3 domain of the extracellular domain of KIT (e.g., human KIT). In some embodiments, an antibody described herein does not immunospecifically binds to a D4 domain of the extracellular domain of KIT (e.g., human KIT). In some embodiments, an anti-KIT antibody described herein does not immunospecifically binds to a D5 domain of the extracellular domain of KIT (e.g., human KIT). In some embodiments, an antibody described herein does not specifically binds to the D4-D5 hinge region of KIT (e.g., human KIT). In certain embodiments, an antibody described herein does not immunospecifically bind to domain D1, D2, or D3 of KIT (e.g., human KIT).

In other specific embodiments, an antibody described herein immunospecifically binds to a monomeric form of KIT (e.g., human KIT). In particular embodiments, an antibody described herein does not immunospecifically bind to a monomeric form of KIT (e.g., human KIT). In specific embodiments, an antibody described herein immunospecifically binds to a dimeric form of KIT (e.g., human KIT). In specific embodiments, an antibody described herein does not bind to a monomeric form of KIT and specifically binds to a dimeric form of KIT or multimeric form of KIT. In certain embodiments, an antibody has higher affinity for a KIT monomer than a KIT dimer. In certain embodiments, an antibody has higher affinity for a KIT monomer than a KIT multimer.

In specific embodiments, an anti-KIT antibody described herein specifically binds to a native isoform or native variant of KIT (that is a naturally occurring isoform or variant of KIT in an animal (e.g., monkey, mouse, goat, donkey, dog, cat, rabbit, pig, rat, human, frog, or bird) that can be isolated from an animal, preferably a human). In particular embodiments, an antibody described herein immunospecifically binds to human KIT or a fragment thereof. In specific embodiments, an anti-KIT antibody described herein specifically binds to human KIT or a fragment thereof and does not specifically bind to non-human KIT (e.g., monkey, mouse, goat, donkey, dog, cat, rabbit, pig, rat, or bird) or a fragment thereof. In certain embodiments, an antibody described herein specifically binds to v-Kit or a fragment thereof (see, e.g., Besmer et al., Nature, 1986, 320:415-21).

In some embodiments, an antibody described herein immunospecifically binds to a KIT antigen comprising one or more (e.g., 1, 2, 3, 4, 5, 6, 7, 8, 9, 10, 15 or 20) amino acid substitutions, deletions, or additions of the amino acid sequence depicted by SEQ ID NO: 170 or 171. In some embodiments, an antibody described herein immunospecifically binds to a KIT antigen comprising at most 10 amino acid substitutions, deletions, or additions; at most 8 amino acid substitutions, deletions, or additions; at most 7 amino acid substitutions, deletions, or additions; at most 6 conservative amino acid substitutions, deletions, or additions; or at most 5 amino acid substitutions, deletions, or additions of the amino acid sequence depicted by SEQ ID NO: 170 or 171.

In some embodiments, an antibody described herein immunospecifically binds to a KIT antigen comprising one or more (e.g., 1, 2, 3, 4, 5, 6, 7, 8, 9, or 10) conservative amino acid substitutions of the amino acid sequence depicted by SEQ ID NO: 170 or 171. In some embodiments, an antibody described herein immunospecifically binds to a KIT antigen comprising at most 10 conservative amino acid substitutions, at most 8 conservative amino acid substitutions, at most 7 conservative amino acid substitutions, at most 6 conservative amino acid substitutions, or at most 5 conservative amino acid substitutions of the amino acid sequence depicted by SEQ ID NO: 170 or 171.

In certain embodiments, an antibody described herein does not immunospecifically bind to a transmembrane region of KIT (e.g., human KIT). In some embodiments, an antibody described herein does not immunospecifically bind to a cytoplasmic juxtamembrane domain of KIT (e.g., human KIT). In some embodiments, an antibody described herein does not immunospecifically bind to a cytoplasmic domain of KIT (e.g., human KIT). In certain embodiments, an antibody described herein does not immunospecifically bind to a split cytoplasmic kinase domain of KIT (e.g., human KIT). In certain embodiments, an antibody described herein does not immunospecifically bind to a tyrosine phosphorylated epitope of KIT (e.g., human KIT).

In a particular embodiment, an antibody described herein is not an antibody described by International Patent Application No. WO 2008/153926, the contents of which are incorporated herein by reference in its entirety. In another particular embodiment, an antibody described herein does not immunospecifically bind to a KIT epitope described by International Patent Application No. WO 2008/153926.

In certain embodiments, an anti-KIT antibody described herein is not an antibody selected from the group consisting of: SR-1 antibody (see U.S. Patent Application Publication No. US 2007/0253951 A1; International Patent Application Publication No. WO 2007/127317); anti-KIT antibody obtained from hybridoma cell lines DSM ACC 2007, DSM ACC 2008, or DSM ACC 2009, which have been deposited at the Deutsche Sammlung von Mikroorganismen und Zellkulturen GmbH, DSM, Mascheroder Weg 1b, D-38124 Braumschweig, Germany (see U.S. Pat. No. 5,545,533; International Patent Application Publication No. WO 92/021766); antibody produced by hybridoma cell line DSM ACC 2247 (or A3C6E2; Deposit No. DSM ACC 2247, at the Deutsche Sammlung von Mikroorganismen und Zellkulturen GmbH, DSM, Mascheroder Weg 1b, D-38124 Braumschweig, Germany) (see U.S. Pat. No. 5,808,002); and anti-KIT antibodies designated K27, K44, K45, K49, K57, K69, and K94 (see, e.g., Blechman et al., Stem Cells, 1993, 11:12-21; Blechman et al., Cell, 1995, 80:103-113; Lev et al., Mol. Cell. Biol., 1993, 13:2224-2234; and European Patent Application Publication No. EP0548867 A2). In a particular embodiment, an antibody described herein does not comprise one or more CDRs (e.g., 3 VL CDRs and/or 3 VH CDRs) of an antibody selected from such group. In another embodiment, an antibody described herein is not competitively blocked (e.g., competitively blocked in a dose-dependent manner) by one of those antibodies, for example, as determined by competition binding assays (e.g., ELISAs).

In a specific embodiment, an antibody described herein is not an antibody produced by the hybridoma (BA7.3C.9) having the American Type Culture Collection (ATCC) Accession number HB10716, as described for example in U.S. Pat. No. 5,919,911 or U.S. Pat. No. 5,489,516. In another specific embodiment, an antibody described herein does not comprise the CDRs (e.g., VL CDR1, VL CDR2, VL CDR3, VH CDR1, VH CDR2, and/or VH CDR3) of the antibody produced by the hybridoma (BA7.3C.9) having the American Type Culture Collection (ATCC) Accession number HB10716, as described for example in U.S. Pat. No. 5,919,911 or U.S. Pat. No. 5,489,516. In another specific embodiment, an antibody described herein does not comprise the CDRs of the SR-1 antibody described for example in U.S. Pat. No. 5,919,911 or U.S. Pat. No. 5,489,516 or U.S. Patent Application Publication No. US 2007/0253951 A1 (see, e.g., ¶ [0032] or ¶ [0023]). In a further embodiment, an antibody described herein is not a humanized antibody of the antibody produced by the hybridoma (BA7.3C.9) having the American Type Culture Collection (ATCC) Accession number HB10716, as described for example in U.S. Pat. No. 5,919,911 or U.S. Pat. No. 5,489,516.

In a specific embodiment, an antibody described herein is not the humanized antibodies of the SR-1 antibody as described in U.S. Patent Application Publication No. US 2007/0253951 A1. In a specific embodiment, an antibody described herein does not comprise one or more amino acid sequences selected from the group consisting of: SEQ ID NO: 2, SEQ ID NO: 4, SEQ ID NO: 6, SEQ ID NO: 8, and SEQ ID NO: 10 referenced in U.S. Patent Application Publication No. US 2007/0253951 A1. In a particular embodiment, an antibody described herein does not comprise the amino acid sequences of SEQ ID NOs: 2 and 4 or of SEQ ID NOs: 2 and 6 referenced in U.S. Patent Application Publication No. US 2007/0253951 A1. In a specific embodiment, an antibody described herein does not comprise one or more amino acid sequence that is at least 90% identical to the amino acid sequence selected from the group consisting of: SEQ ID NO: 2, SEQ ID NO: 4, SEQ ID NO: 6, SEQ ID NO: 8, and SEQ ID NO: 10 referenced in U.S. Patent Application Publication No. US 2007/0253951 A1. In a particular embodiment, an antibody described herein does not comprise one or more CDRs described in U.S. Patent Application Publication No. US 2007/0253951 A1, for example, amino acids 44 to 58 of SEQ ID NO: 8 (VL CDR1 of antibody SR-1; RASESVDIYGNSFMH), amino acids 74 to 80 of SEQ ID NO: 8 (VL CDR2 of antibody SR-1; LASNLES), amino acids 111 to 121 of SEQ ID NO: 8 (VL CDR3 of antibody SR-1; QQNNEDPYT), amino acids 50 to 54 of SEQ ID NO: (VH CDR1 of antibody SR-1; SYNMH), amino acids 69 to 85 of SEQ ID NO: 10 (VH CDR2 of antibody SR-1; VIYSGNGDTSYNQKFKG), and/or amino acids 118 to 125 of SEQ ID NO: (VH CDR3 of antibody SR-1; RDTRFGN), where SEQ ID NOs: 8 and 10 are those referenced in U.S. Patent Application Publication No. US 2007/0253951 A1 (see, e.g., ¶ [0032] or ¶ [0023]). In a particular embodiment, an antibody described herein does not comprise one or more CDRs described in U.S. Patent Application Publication No. US 2007/0253951 A1, for example, amino acids 43 to 58 of SEQ ID NO: 2 (VL CDR1), amino acids 74 to 80 of SEQ ID NO: 2 (VL CDR2), amino acids 113 to 121 of SEQ ID NO: 2 (VL CDR3), amino acids 50 to 54 of SEQ ID NO: 4 (VH CDR1), amino acids 69 to 85 of SEQ ID NO: 4 (VH CDR2), and/or amino acids 118 to 125 of SEQ ID NO: 4 (VH CDR3), where SEQ ID NOs: 2 and 4 are those referenced in U.S. Patent Application Publication No. US 2007/0253951 A1. In a particular embodiment, an antibody described herein is not a humanized antibody of antibody SR-1 as described in U.S. Patent Application Publication No. US 2007/0253951 A1.

In a specific embodiment, an antibody described herein is not an antibody selected from the group consisting of: antibody Anti-S100, ACK2, and ACK4 described in U.S. Pat. No. 6,989,248 or U.S. Pat. No. 7,449,309. In a specific embodiment, an antibody described herein does not comprise one or more CDRs (e.g., 3 VL CDRs and/or 3 VH CDRs) of an antibody selected from the group consisting of: antibody Anti-S100, ACK2, and ACK4 described in U.S. Pat. No. 6,989,248 or U.S. Pat. No. 7,449,309.

In particular aspects, provided herein are antibodies (e.g., a human antibody), which immunospecifically bind to a KIT polypeptide (e.g., the D4/D5 region of KIT, for example, human KIT) and comprise an amino acid sequence as described herein, as well as antibodies which compete (e.g., compete in a dose-dependent manner) with such antibodies for binding to a KIT polypeptide.

In a particular embodiment, an antibody described herein comprises a VL chain region having the amino acid sequence of SEQ ID NO: 176 (VL domain of Ab1), and comprises a VH chain region having the amino acid sequence of SEQ ID NO: 188 (VH domain of Ab1). In a particular embodiment, an antibody described herein comprises a VL chain region having the amino acid sequence of SEQ ID NO: 350 (VL domain of Ab1), and comprises a VH chain region having the amino acid sequence of SEQ ID NO: 188 (VH domain of Ab1). In a particular embodiment, an antibody described herein comprises a VL chain region having the amino acid sequence of SEQ ID NO: 350 (VL domain of Ab1) and an additional R (Arg) amino acid at the C-terminus, and comprises a VH chain region having the amino acid sequence of SEQ ID NO: 188 (VH domain of Ab1). In a particular embodiment, an antibody described herein comprises a VL chain region having the amino acid sequence of SEQ ID NO: 963 (VL domain of Ab1), and comprises a VH chain region having the amino acid sequence of SEQ ID NO: 188 (VH domain of Ab1). In a particular embodiment, an antibody described herein comprises a VL chain region having the amino acid sequence of SEQ ID NO: 176 starting at the second amino acid residue of SEQ ID NO: 176.

In a particular embodiment, an antibody described herein comprises a VL chain region having the amino acid sequence of SEQ ID NO: 177 (VL domain of Ab2), and comprises a VH chain region having the amino acid sequence of SEQ ID NO: 189 (VH domain of Ab2). In a particular embodiment, an antibody described herein comprises a VL chain region having the amino acid sequence of SEQ ID NO: 178 (VL domain of Ab3), and comprises a VH chain region having the amino acid sequence of SEQ ID NO: 190 (VH domain of Ab3). In a particular embodiment, an antibody described herein comprises a VL chain region having the amino acid sequence of SEQ ID NO: 179 (VL domain of Ab4), and comprises a VH chain region having the amino acid sequence of SEQ ID NO: 191 (VH domain of Ab4). In a particular embodiment, an antibody described herein comprises a VL chain region having the amino acid sequence of SEQ ID NO: 180 (VL domain of Ab5), and comprises a VH chain region having the amino acid sequence of SEQ ID NO: 192 (VH domain of Ab5). In a particular embodiment, an antibody described herein comprises a VL chain region having the amino acid sequence of SEQ ID NO: 181 (VL domain of Ab6), and comprises a VH chain region having the amino acid sequence of SEQ ID NO: 193 (VH domain of Ab6). In a particular embodiment, an antibody described herein comprises a VL chain region having the amino acid sequence of SEQ ID NO: 182 (VL domain of Ab7), and comprises a VH chain region having the amino acid sequence of SEQ ID NO: 194 (VH domain of Ab7). In a particular embodiment, an antibody described herein comprises a VL chain region having the amino acid sequence of SEQ ID NO: 183 (VL domain of Ab8), and comprises a VH chain region having the amino acid sequence of SEQ ID NO: 195 (VH domain of Ab8). In a particular embodiment, an antibody described herein comprises a VL chain region having the amino acid sequence of SEQ ID NO: 184 (VL domain of Ab9), and comprises a VH chain region having the amino acid sequence of SEQ ID NO: 196 (VH domain of Ab9). In a particular embodiment, an antibody described herein comprises a VL chain region having the amino acid sequence of SEQ ID NO: 185 (VL domain of Ab10), and comprises a VH chain region having the amino acid sequence of SEQ ID NO: 197 (VH domain of Ab10). In a particular embodiment, an antibody described herein comprises a VL chain region having the amino acid sequence of SEQ ID NO: 186 (VL domain of Ab11), and comprises a VH chain region having the amino acid sequence of SEQ ID NO: 198 (VH domain of Ab11). In a particular embodiment, an antibody described herein comprises a VL chain region having the amino acid sequence of SEQ ID NO: 187 (VL domain of Ab12), and comprises a VH chain region having the amino acid sequence of SEQ ID NO: 199 (VH domain of Ab12). In certain embodiments, any of these antibodies can comprise a variable heavy chain and a variable light chain, for example, a separate variable heavy chain and a separate variable light chain. In a specific embodiment, an antibody described herein comprises a VL chain region having the amino acid sequence of any one of SEQ ID NOs: 176-187 starting at the second amino acid residue of SEQ ID NOs: 176-187, respectively. In a particular embodiment, an antibody described herein comprises a VL chain region having the amino acid sequence of SEQ ID NO: 176, except for the first amino acid residue of SEQ ID NO: 176. In a particular embodiment, an antibody described herein comprises a VL chain region having the amino acid sequence of SEQ ID NO: 350. In a specific embodiment, the position (i.e., boundary) of a VL chain region described herein relative to the constant region may change by one, two, three, or four amino acid positions so long as immunospecific binding to KIT (e.g., the D4/D5 region of human KIT) is maintained. In a specific embodiment, the position (i.e., boundary) of a VH chain region described herein relative to the constant region may change by one, two, three, or four amino acid positions so long as immunospecific binding to KIT (e.g., the D4/D5 region of human KIT) is maintained. In a certain embodiment, the first amino acid at the N-terminus of a VH chain region described herein (e.g., VH chain region of any one of antibodies Ab1-Ab20) is modified, or removed, without affecting the function to immunospecifically bind to KIT. For example, in a particular embodiment, an antibody described herein comprises a VL chain region having the amino acid sequence of any one of SEQ ID NOs: 176-187 starting at the second amino acid residue of SEQ ID NOs: 176-187, respectively, and/or the VL chain region further comprises an additional R (Arg) amino acid residue at the C-terminus.

In a particular embodiment, an antibody described herein comprises a VL chain region having the amino acid sequence of any one of SEQ ID NOs: 312-319 (VL domain of Ab13-Ab20), and comprises a VH chain region having the amino acid sequence of any one of SEQ ID NOs: 320-327 (VH domain of Ab13-Ab20). In a particular embodiment, an antibody described herein comprises a VL chain region having the amino acid sequence of any one of SEQ ID NOs: 312-319 (VL domain of Ab13-Ab20) starting at the second amino acid residue of SEQ ID NOs: 312-319, respectively, and/or the VL chain region further comprises an additional R (Arg) amino acid residue at the C-terminus.

In a particular embodiment, an antibody described herein comprises a VL chain region having the amino acid sequence of SEQ ID NO: 328 (VL domain of Ab21), and comprises a VH chain region having the amino acid sequence of SEQ ID NO: 329 (VH domain of Ab21). In a particular embodiment, an antibody described herein (i) comprises a VL chain region having the amino acid sequence of SEQ ID NO: 328 (VL domain of Ab21), optionally the VL chain region further comprises an additional R (Arg) amino acid residue at the C-terminus, and (ii) comprises a VH chain region having the amino acid sequence of SEQ ID NO: 329 (VH domain of Ab21). In a certain embodiment, an antibody described herein (i) comprises a VL chain region having the amino acid sequence of SEQ ID NO: 176 (VL domain of Ab1), the amino acid sequence of SEQ ID NO: 176 (VL domain of Ab1) starting at the second amino acid residue of SEQ ID NO: 176, or SEQ ID NO: 328 (VL domain of Ab21), optionally the VL chain region further comprises an additional R (Arg) amino acid residue at the C-terminus, and (ii) comprises a VH chain region having the amino acid sequence of SEQ ID NO: 188 (VH domain of Ab1). In a certain embodiment, an antibody described herein comprises (i) a VL chain region having the amino acid sequence of SEQ ID NO: 350 (VL domain of Ab1), optionally the VL chain region further comprises an additional R (Arg) amino acid residue at the C-terminus and (ii) a VH chain region having the amino acid sequence of SEQ ID NO: 188 (VH domain of Ab1). In a certain embodiment, an antibody described herein comprises (i) a VL chain region having the amino acid sequence of SEQ ID NO: 963 or 964, and (ii) a VH chain region having the amino acid sequence of SEQ ID NO: 188 (VH domain of Ab1).

In certain embodiments, an antibody described herein comprises a variable light (VL) chain region comprising an amino acid sequence described herein (e.g., see FIG. 2). In certain embodiments, an antibody described herein comprises a VL chain region having the amino acid sequence of SEQ ID NO: 176 or 350 (Ab1), SEQ ID NO: 177 (Ab2), SEQ ID NO: 178 (Ab3), SEQ ID NO: 179 (Ab4), SEQ ID NO: 180 (Ab5), SEQ ID NO: 181 (Ab6), SEQ ID NO: 182 (Ab7), SEQ ID NO: 183 (Ab8), SEQ ID NO: 184 (Ab9), SEQ ID NO: 185 (Ab10), SEQ ID NO: 186 (Ab11), SEQ ID NO: 187 (Ab12), SEQ ID NO: 328 (Ab21), or SEQ ID NO: 176 (VL domain of Ab1) starting at the second amino acid residue of SEQ ID NO: 176, wherein the antibody immunospecifically binds to a KIT polypeptide, e.g., a human KIT polypeptide, for example, a D4/D5 region of KIT (e.g., human KIT), for example SEQ ID NO: 171. In a particular embodiment, the VL chain region further comprises an R (Arg) amino acid residue at the C-terminus. In certain embodiments, any of these antibodies can comprise a variable variable light chain, for example, a separate variable light chain.

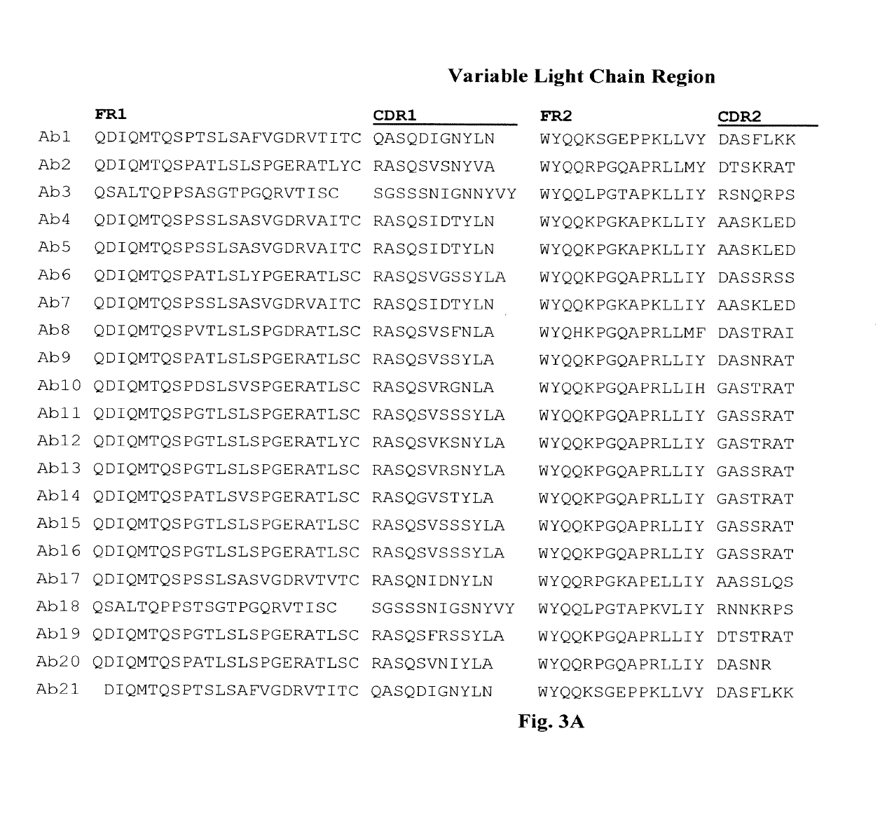

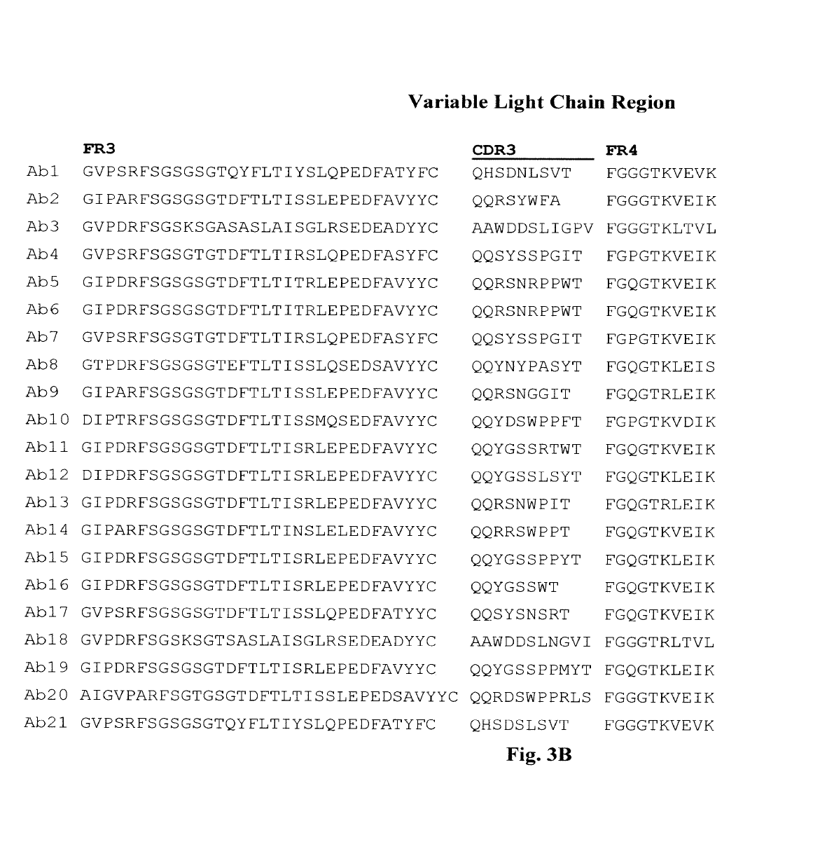

In certain embodiments, an antibody described herein comprises a variable heavy (VH) chain region comprising an amino acid sequence described herein (e.g., see FIG. 3). In certain embodiments, an antibody described herein comprises a VH chain region having the amino acid sequence of SEQ ID NO: 188 (Ab1), SEQ ID NO: 189 (Ab2), SEQ ID NO: 190 (Ab3), SEQ ID NO: 191 (Ab4), SEQ ID NO: 192 (Ab5), SEQ ID NO: 193 (Ab6), SEQ ID NO: 194 (Ab7), SEQ ID NO: 195 (Ab8), SEQ ID NO: 196 (Ab9), SEQ ID NO: 197 (Ab10), SEQ ID NO: 198 (Ab11), SEQ ID NO: 199 (Ab12), or SEQ ID NO: 329 (Ab21), wherein the antibody immunospecifically binds to a KIT polypeptide, e.g., a human KIT polypeptide, for example, a D4/D5 region of KIT (e.g., human KIT), for example SEQ ID NO: 171. In certain embodiments, any of these antibodies can comprise a variable variable heavy chain, for example, a separate variable heavy chain.

In certain embodiments, an antibody described herein comprises one or more VL CDRs having the amino acid sequence described herein (e.g., see Tables 1A, 1B and 1C), wherein the antibody immunospecifically binds to a KIT polypeptide, e.g., a human KIT polypeptide, for example, a D4/D5 region of KIT (e.g., human KIT), for example SEQ ID NO: 171. In a specific embodiment, an antibody described herein comprises VL CDR1 and VL CDR2, or VL CDR2 and VL CDR3, or VL CDR1 and VL CDR3, from any one of the antibodies described in Tables 1A-1C. In particular embodiments, the VL CDR1 has the amino acid sequence of any one of SEQ ID NOs: 2-13. In certain embodiments, the VL CDR2 has the amino acid sequence of any one of SEQ ID NOs: 14-25. In specific embodiments, the VL CDR3 has the amino acid sequence of any one of SEQ ID NOs: 26-37. In certain embodiments, an antibody described herein comprises at least two VL CDRs from the same antibody sequence depicted at Tables 1A, 1B and 1C, e.g., comprises VL CDR1 and VL CDR2 of Ab1. In certain embodiments, an antibody described herein comprises a VL CDR1, VL CDR2, and/or VL CDR3 from the same antibody sequence depicted at Tables 1A, 1B and 1C, e.g., comprises VL CDR1, VL CDR2, and VL CDR3 of Ab1.

In particular embodiments, the VL CDR1 has the amino acid sequence of any one of SEQ ID NOs: 200-207. In certain embodiments, the VL CDR2 has the amino acid sequence of any one of SEQ ID NOs: 208-215. In specific embodiments, the VL CDR3 has the amino acid sequence of any one of SEQ ID NOs: 216-223. In a specific embodiment, an antibody described herein comprises a VL chain region comprising (i) a VL CDR1 having the amino acid sequence of any one of SEQ ID NOs: 200-207; (ii) a VL CDR2 having the amino acid sequence of any one of SEQ ID NOs: 208-215; and (iii) a VL CDR3 having the amino acid sequence of any one of SEQ ID NO: 216-223, wherein the antibody immunospecifically binds to a KIT polypeptide, e.g., a human KIT polypeptide, for example, a D4/D5 region of KIT (e.g., human KIT), for example SEQ ID NO: 171.

In a specific embodiment, an antibody described herein comprises a VL chain region comprising (i) a VL CDR1 having the amino acid sequence of SEQ ID NO: 2 (VL CDR1 of Ab1) or SEQ ID NO: 3 (VL CDR1 of Ab2); (ii) a VL CDR2 having the amino acid sequence of SEQ ID NO: 14 (VL CDR2 of Ab1) or SEQ ID NO: 15 (VL CDR2 of Ab2); and (iii) a VL CDR3 having the amino acid sequence of SEQ ID NO: 26 (VL CDR3 of Ab1) or SEQ ID NO: 27 (VL CDR3 of Ab2), wherein the antibody immunospecifically binds to a KIT polypeptide, e.g., a human KIT polypeptide, for example, a D4/D5 region of KIT (e.g., human KIT), for example SEQ ID NO: 171.

In certain embodiments, an antibody described herein (e.g., human antibody) comprises a VL chain region comprising a VL CDR1 having the amino acid sequence of SEQ ID NO: 2 (VL CDR1 of Ab1), a VL CDR2 having the amino acid sequence of SEQ ID NO: 14 (VL CDR2 of Ab1), and a VL CDR3 having the amino acid sequence of SEQ ID NO: 26 (VL CDR3 of Ab1). In certain embodiments, an antibody described herein (e.g., human antibody) comprises a VL chain region comprising a VL CDR1 having the amino acid sequence of SEQ ID NO: 3 (VL CDR1 of Ab2), a VL CDR2 having the amino acid sequence of SEQ ID NO: 15 (VL CDR2 of Ab2), and a VL CDR3 having the amino acid sequence of SEQ ID NO: 27 (VL CDR3 of Ab2). In certain embodiments, an antibody described herein (e.g., human antibody) comprises a VL chain region comprising a VL CDR1 having the amino acid sequence of SEQ ID NO: 4 (VL CDR1 of Ab3), a VL CDR2 having the amino acid sequence of SEQ ID NO: 16 (VL CDR2 of Ab3), and a VL CDR3 having the amino acid sequence of SEQ ID NO: 28 (VL CDR3 of Ab3). In certain embodiments, an antibody described herein (e.g., human antibody) comprises a VL chain region comprising a VL CDR1 having the amino acid sequence of SEQ ID NO: 5 (VL CDR1 of Ab4), a VL CDR2 having the amino acid sequence of SEQ ID NO: 17 (VL CDR2 of Ab4), and a VL CDR3 having the amino acid sequence of SEQ ID NO: 29 (VL CDR3 of Ab4). In certain embodiments, an antibody described herein (e.g., human antibody) comprises a VL chain region comprising a VL CDR1 having the amino acid sequence of SEQ ID NO: 6 (VL CDR1 of Ab5), a VL CDR2 having the amino acid sequence of SEQ ID NO: 18 (VL CDR2 of Ab5), and a VL CDR3 having the amino acid sequence of SEQ ID NO: 30 (VL CDR3 of Ab5). In certain embodiments, an antibody described herein (e.g., human antibody) comprises a VL chain region comprising a VL CDR1 having the amino acid sequence of SEQ ID NO: 7 (VL CDR1 of Ab6), a VL CDR2 having the amino acid sequence of SEQ ID NO: 19 (VL CDR2 of Ab6), and a VL CDR3 having the amino acid sequence of SEQ ID NO: 31 (VL CDR3 of Ab6). In certain embodiments, an antibody described herein (e.g., human antibody) comprises a VL chain region comprising a VL CDR1 having the amino acid sequence of SEQ ID NO: 8 (VL CDR1 of Ab7), a VL CDR2 having the amino acid sequence of SEQ ID NO: 20 (VL CDR2 of Ab7), and a VL CDR3 having the amino acid sequence of SEQ ID NO: 32 (VL CDR3 of Ab7). In certain embodiments, an antibody described herein (e.g., human antibody) comprises a VL chain region comprising a VL CDR1 having the amino acid sequence of SEQ ID NO: 9 (VL CDR1 of Ab8), a VL CDR2 having the amino acid sequence of SEQ ID NO: 21 (VL CDR2 of Ab8), and a VL CDR3 having the amino acid sequence of SEQ ID NO: 33 (VL CDR3 of Ab8). In certain embodiments, an antibody described herein (e.g., human antibody) comprises a VL chain region comprising a VL CDR1 having the amino acid sequence of SEQ ID NO: 10 (VL CDR1 of Ab9), a VL CDR2 having the amino acid sequence of SEQ ID NO: 22 (VL CDR2 of Ab9), and a VL CDR3 having the amino acid sequence of SEQ ID NO: 34 (VL CDR3 of Ab9). In certain embodiments, an antibody described herein (e.g., human antibody) comprises a VL chain region comprising a VL CDR1 having the amino acid sequence of SEQ ID NO: 11 (VL CDR1 of Ab10), a VL CDR2 having the amino acid sequence of SEQ ID NO: 23 (VL CDR2 of Ab10), and a VL CDR3 having the amino acid sequence of SEQ ID NO: 35 (VL CDR3 of Ab10). In certain embodiments, an antibody described herein (e.g., human antibody) comprises a VL chain region comprising a VL CDR1 having the amino acid sequence of SEQ ID NO: 12 (VL CDR1 of Ab11), a VL CDR2 having the amino acid sequence of SEQ ID NO: 24 (VL CDR2 of Ab11), and a VL CDR3 having the amino acid sequence of SEQ ID NO: 36 (VL CDR3 of Ab11). In certain embodiments, an antibody described herein (e.g., human antibody) comprises a VL chain region comprising a VL CDR1 having the amino acid sequence of SEQ ID NO: 13 (VL CDR1 of Ab12), a VL CDR2 having the amino acid sequence of SEQ ID NO: 25 (VL CDR2 of Ab12), and a VL CDR3 having the amino acid sequence of SEQ ID NO: 37 (VL CDR3 of Ab12). In certain embodiments, an antibody described herein (e.g., human antibody) comprises a VL chain region comprising a VL CDR1 having the amino acid sequence of SEQ ID NO: 330 (VL CDR1 of Ab21), a VL CDR2 having the amino acid sequence of SEQ ID NO: 331 (VL CDR2 of Ab21), and a VL CDR3 having the amino acid sequence of SEQ ID NO: 332 (VL CDR3 of Ab21). In certain embodiments, an antibody described herein (e.g., human antibody) comprises a VL chain region comprising a VL CDR1 having the amino acid sequence of SEQ ID NO: 2 (VL CDR1 of Ab1), a VL CDR2 having the amino acid sequence of SEQ ID NO: 14 (VL CDR2 of Ab1), and a VL CDR3 having the amino acid sequence of SEQ ID NO: 26 (VL CDR3 of Ab1) or SEQ ID NO: 332 (VL CDR3 of Ab21). In specific embodiments, any of these antibodies can immunospecifically bind to a KIT polypeptide, e.g., a human KIT polypeptide, for example, a D4/D5 region of KIT (e.g., human KIT), for example SEQ ID NO: 171.

In certain embodiments, an antibody described herein comprises one or more VH CDRs having the amino acid sequence described herein (e.g., see Tables 3A, 3B and 3C), wherein the antibody immunospecifically binds to a KIT polypeptide, e.g., a human KIT polypeptide, for example, a D4/D5 region of KIT (e.g., human KIT), for example SEQ ID NO: 171. In a specific embodiment, an antibody described herein comprises VH CDR1 and VH CDR2, or VH CDR2 and VH CDR3, or VH CDR1 and VH CDR3, from any one of the antibodies described in Tables 3A-3C. In particular embodiments, the VH CDR1 has the amino acid sequence of any one of SEQ ID NOs: 86-97. In certain embodiments, the VH CDR2 has the amino acid sequence of any one of SEQ ID NOs: 98-109. In specific embodiments, the VH CDR3 has the amino acid sequence of any one of SEQ ID NOs: 110-121. In certain embodiments, an antibody described herein comprises at least two VH CDRs from the same antibody sequence depicted at Tables 3A, 3B, and 3C, e.g., comprises VH CDR1 and VH CDR2 of Ab1. In certain embodiments, an antibody described herein comprises a VH CDR1, VH CDR2, and/or VH CDR3 from the same antibody sequence depicted at Tables 3A, 3B and 3C, e.g., comprises VH CDR1, VH CDR2, and VH CDR3 of Ab1.

In particular embodiments, the VH CDR1 has the amino acid sequence of any one of SEQ ID NOs: 256-263. In certain embodiments, the VH CDR2 has the amino acid sequence of any one of SEQ ID NOs: 264-271. In specific embodiments, the VH CDR3 has the amino acid sequence of any one of SEQ ID NOs: 272-279. In a specific embodiment, an antibody described herein comprises a VH chain region comprising (i) a VH CDR1 having the amino acid sequence of any one of SEQ ID NOs: 256-263; (ii) a VH CDR2 having the amino acid sequence of any one of SEQ ID NOs: 264-271; and (iii) a VH CDR3 having the amino acid sequence of any one of SEQ ID NOs: 272-279, wherein the antibody immunospecifically binds to a KIT polypeptide, e.g., a human KIT polypeptide, for example, a D4/D5 region of KIT (e.g., human KIT), for example SEQ ID NO: 171.

In a specific embodiment, an antibody described herein comprises a VH chain region comprising (i) a VH CDR1 having the amino acid sequence of SEQ ID NO: 86 or SEQ ID NO: 87; (ii) a VH CDR2 having the amino acid sequence of SEQ ID NO: 98 or SEQ ID NO: 99; and (iii) a VH CDR3 having the amino acid sequence of SEQ ID NO: 110 or SEQ ID NO: 111, wherein the antibody immunospecifically binds to a KIT polypeptide, e.g., a human KIT polypeptide, for example, a D4/D5 region of KIT (e.g., human KIT), for example SEQ ID NO: 171.

In certain embodiments, an antibody (e.g., human antibody) comprises a VH chain region comprising a VH CDR1 having the amino acid sequence of SEQ ID NO: 86 (VH CDR1 of Ab1), a VH CDR2 having the amino acid sequence of SEQ ID NO: 98 (VH CDR2 of Ab1), and a VH CDR3 having the amino acid sequence of SEQ ID NO: 110 (VH CDR3 of Ab1). In certain embodiments, an antibody (e.g., human antibody) comprises a VH chain region comprising a VH CDR1 having the amino acid sequence of SEQ ID NO: 87 (VH CDR1 of Ab2), a VH CDR2 having the amino acid sequence of SEQ ID NO: 99 (VH CDR2 of Ab2), and a VH CDR3 having the amino acid sequence of SEQ ID NO: 111 (VH CDR3 of Ab2). In certain embodiments, an antibody (e.g., human antibody) comprises a VH chain region comprising a VH CDR1 having the amino acid sequence of SEQ ID NO: 88 (VH CDR1 of Ab3), a VH CDR2 having the amino acid sequence of SEQ ID NO: 100 (VH CDR2 of Ab3), and a VH CDR3 having the amino acid sequence of SEQ ID NO: 112 (VH CDR3 of Ab3). In certain embodiments, an antibody (e.g., human antibody) comprises a VH chain region comprising a VH CDR1 having the amino acid sequence of SEQ ID NO: 89 (VH CDR1 of Ab4), a VH CDR2 having the amino acid sequence of SEQ ID NO: 101 (VH CDR2 of Ab4), and a VH CDR3 having the amino acid sequence of SEQ ID NO: 113 (VH CDR3 of Ab4). In certain embodiments, an antibody (e.g., human antibody) comprises a VH chain region comprising a VH CDR1 having the amino acid sequence of SEQ ID NO: 90 (VH CDR1 of Ab5), a VH CDR2 having the amino acid sequence of SEQ ID NO: 102 (VH CDR2 of Ab5), and a VH CDR3 having the amino acid sequence of SEQ ID NO: 114 (VH CDR3 of Ab5). In certain embodiments, an antibody (e.g., human antibody) comprises a VH chain region comprising a VH CDR1 having the amino acid sequence of SEQ ID NO: 91 (VH CDR1 of Ab6), a VH CDR2 having the amino acid sequence of SEQ ID NO: 103 (VH CDR2 of Ab6), and a VH CDR3 having the amino acid sequence of SEQ ID NO: 115 (VH CDR3 of Ab6). In certain embodiments, an antibody (e.g., human antibody) comprises a VH chain region comprising a VH CDR1 having the amino acid sequence of SEQ ID NO: 92 (VH CDR1 of Ab7), a VH CDR2 having the amino acid sequence of SEQ ID NO: 104 (VH CDR2 of Ab7), and a VH CDR3 having the amino acid sequence of SEQ ID NO: 116 (VH CDR3 of Ab7). In certain embodiments, an antibody (e.g., human antibody) comprises a VH chain region comprising a VH CDR1 having the amino acid sequence of SEQ ID NO: 93 (VH CDR1 of Ab8), a VH CDR2 having the amino acid sequence of SEQ ID NO: 105 (VH CDR2 of Ab8), and a VH CDR3 having the amino acid sequence of SEQ ID NO: 117 (VH CDR3 of Ab8). In certain embodiments, an antibody (e.g., human antibody) comprises a VH chain region comprising a VH CDR1 having the amino acid sequence of SEQ ID NO: 94 (VH CDR1 of Ab9), a VH CDR2 having the amino acid sequence of SEQ ID NO: 106 (VH CDR2 of Ab9), and a VH CDR3 having the amino acid sequence of SEQ ID NO: 118 (VH CDR3 of Ab9). In certain embodiments, an antibody (e.g., human antibody) comprises a VH chain region comprising a VH CDR1 having the amino acid sequence of SEQ ID NO: 95 (VH CDR1 of Ab10), a VH CDR2 having the amino acid sequence of SEQ ID NO: 107 (VH CDR2 of Ab10), and a VH CDR3 having the amino acid sequence of SEQ ID NO: 119 (VH CDR3 of Ab10). In certain embodiments, an antibody (e.g., human antibody) comprises a VH chain region comprising a VH CDR1 having the amino acid sequence of SEQ ID NO: 96 (VH CDR1 of Ab11), a VH CDR2 having the amino acid sequence of SEQ ID NO: 108 (VH CDR2 of Ab11), and a VH CDR3 having the amino acid sequence of SEQ ID NO: 120 (VH CDR3 of Ab11). In certain embodiments, an antibody (e.g., human antibody) comprises a VH chain region comprising a VH CDR1 having the amino acid sequence of SEQ ID NO: 97 (VH CDR1 of Ab12), a VH CDR2 having the amino acid sequence of SEQ ID NO: 109 (VH CDR2 of Ab12), and a VH CDR3 having the amino acid sequence of SEQ ID NO: 121 (VH CDR3 of Ab12). In certain embodiments, an antibody (e.g., human antibody) comprises a VH chain region comprising a VH CDR1 having the amino acid sequence of SEQ ID NO: 337 (VH CDR1 of Ab21), a VH CDR2 having the amino acid sequence of SEQ ID NO: 338 (VH CDR2 of Ab21), and a VH CDR3 having the amino acid sequence of SEQ ID NO: 339 (VH CDR3 of Ab21). In specific embodiments, any of these antibodies can immunospecifically bind to a KIT polypeptide, e.g., a human KIT polypeptide, for example, a D4/D5 region of KIT (e.g., human KIT), for example SEQ ID NO: 171.

In another specific embodiment, an antibody (e.g., human antibody) described herein comprises (i) a VL chain region comprising a VL CDR1, VL CDR2, and VL CDR3 having the amino acid sequences of SEQ ID NO: 2 (VL CDR1 of Ab1), SEQ ID NO: 14 (VL CDR2 of Ab1), and SEQ ID NO: 26 (VL CDR3 of Ab1), respectively; and (ii) a VH chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively. In another specific embodiment, an antibody (e.g., human antibody) described herein comprises (i) a VL chain region comprising a VL CDR1, VL CDR2, and VL CDR3, wherein the VL CDR1 has the amino acid sequence of SEQ ID NO: 2 (VL CDR1 of Ab1), the VL CDR2 has the amino acid sequence of SEQ ID NO: 14 (VL CDR2 of Ab1), and the VL CDR3 has the amino acid sequence of SEQ ID NO: 26 (VL CDR3 of Ab1) or SEQ ID NO: 332 (VL CDR3 of Ab21), respectively; and (ii) a VH chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively.

In another specific embodiment, an antibody (e.g., human antibody) described herein comprises (i) a VL chain region comprising a VL CDR1, VL CDR2, and VL CDR3 having the amino acid sequences of SEQ ID NO: 3 (VL CDR1 of Ab2), SEQ ID NO: 15 (VL CDR2 of Ab2), and SEQ ID NO: 27 (VL CDR3 of Ab2), respectively; and (ii) a VH chain region comprising a VH CDR1, VH CDR2, and VH CDR3 having the amino acid sequences of SEQ ID NO: 87 (VH CDR1 of Ab2), SEQ ID NO: 99 (VH CDR2 of Ab3), and SEQ ID NO: 111 (VH CDR3 of Ab2), respectively.

In another specific embodiment, an antibody (e.g., human antibody) described herein comprises (i) a VL chain region comprising a VL CDR1, VL CDR2, and VL CDR3 having the amino acid sequences of SEQ ID NO: 4 (VL CDR1 of Ab3), SEQ ID NO: 16 (VL CDR2 of Ab3), and SEQ ID NO: 28 (VL CDR3 of Ab3), respectively; and (ii) a VH chain region comprising a VH CDR1, VH CDR2, and VH CDR3 having the amino acid sequences of SEQ ID NO: 88 (VH CDR1 of Ab3), SEQ ID NO: 100 (VH CDR2 of Ab3), and SEQ ID NO: 112 (VH CDR3 of Ab3), respectively.

In another specific embodiment, an antibody (e.g., human antibody) described herein comprises (i) a VL chain region comprising a VL CDR1, VL CDR2, and VL CDR3 having the amino acid sequences of SEQ ID NO: 5 (VL CDR1 of Ab4), SEQ ID NO: 17 (VL CDR2 of Ab4), and SEQ ID NO: 29 (VL CDR3 of Ab4), respectively; and (ii) a VH chain region comprising a VH CDR1, VH CDR2, and VH CDR3 having the amino acid sequences of SEQ ID NO: 89 (VH CDR1 of Ab4), SEQ ID NO: 101 (VH CDR2 of Ab4), and SEQ ID NO: 113 (VH CDR3 of Ab4), respectively.

In another specific embodiment, an antibody (e.g., human antibody) described herein comprises (i) a VL chain region comprising a VL CDR1, VL CDR2, and VL CDR3 having the amino acid sequences of SEQ ID NO: 6 (VL CDR1 of Ab5), SEQ ID NO: 18 (VL CDR2 of Ab5), and SEQ ID NO: 30 (VL CDR3 of Ab5), respectively; and (ii) a VH chain region comprising a VH CDR1, VH CDR2, and VH CDR3 having the amino acid sequences of SEQ ID NO: 90 (VH CDR1 of Ab5), SEQ ID NO: 102 (VH CDR2 of Ab5), and SEQ ID NO: 114 (VH CDR3 of Ab5), respectively.

In another specific embodiment, an antibody (e.g., human antibody) described herein comprises (i) a VL chain region comprising a VL CDR1, VL CDR2, and VL CDR3 having the amino acid sequences of SEQ ID NO: 7 (VL CDR1 of Ab6), SEQ ID NO: 19 (VL CDR2 of Ab6), and SEQ ID NO: 31 (VL CDR3 of Ab6), respectively; and (ii) a VH chain region comprising a VH CDR1, VH CDR2, and VH CDR3 having the amino acid sequences of SEQ ID NO: 91 (VH CDR1 of Ab6), SEQ ID NO: 103 (VH CDR2 of Ab6), and SEQ ID NO: 115 (VH CDR3 of Ab6), respectively.

In another specific embodiment, an antibody (e.g., human antibody) described herein comprises (i) a VL chain region comprising a VL CDR1, VL CDR2, and VL CDR3 having the amino acid sequences of SEQ ID NO: 8 (VL CDR1 of Ab7), SEQ ID NO: 20 (VL CDR2 of Ab7), and SEQ ID NO: 32 (VL CDR3 of Ab7), respectively; and (ii) a VH chain region comprising a VH CDR1, VH CDR2, and VH CDR3 having the amino acid sequences of SEQ ID NO: 92 (VH CDR1 of Ab7), SEQ ID NO: 104 (VH CDR2 of Ab7), and SEQ ID NO: 116 (VH CDR3 of Ab7), respectively.

In another specific embodiment, an antibody (e.g., human antibody) described herein comprises (i) a VL chain region comprising a VL CDR1, VL CDR2, and VL CDR3 having the amino acid sequences of SEQ ID NO: 9 (VL CDR1 of Ab8), SEQ ID NO: 21 (VL CDR2 of Ab8), and SEQ ID NO: 33 (VL CDR3 of Ab8), respectively; and (ii) a VH chain region comprising a VH CDR1, VH CDR2, and VH CDR3 having the amino acid sequences of SEQ ID NO: 93 (VH CDR1 of Ab8), SEQ ID NO: 105 (VH CDR2 of Ab8), and SEQ ID NO: 117 (VH CDR3 of Ab8), respectively.

In another specific embodiment, an antibody (e.g., human antibody) described herein comprises (i) a VL chain region comprising a VL CDR1, VL CDR2, and VL CDR3 having the amino acid sequences of SEQ ID NO: 10 (VL CDR1 of Ab9), SEQ ID NO: 22 (VL CDR2 of Ab9), and SEQ ID NO: 34 (VL CDR3 of Ab9), respectively; and (ii) a VH chain region comprising a VH CDR1, VH CDR2, and VH CDR3 having the amino acid sequences of SEQ ID NO: 94 (VH CDR1 of Ab9), SEQ ID NO: 106 (VH CDR2 of Ab9), and SEQ ID NO: 118 (VH CDR3 of Ab9), respectively.

In another specific embodiment, an antibody (e.g., human antibody) described herein comprises (i) a VL chain region comprising a VL CDR1, VL CDR2, and VL CDR3 having the amino acid sequences of SEQ ID NO: 11 (VL CDR1 of Ab10), SEQ ID NO: 23 (VL CDR2 of Ab10), and SEQ ID NO: 35 (VL CDR3 of Ab10), respectively; and (ii) a VH chain region comprising a VH CDR1, VH CDR2, and VH CDR3 having the amino acid sequences of SEQ ID NO: 95 (VH CDR1 of Ab10), SEQ ID NO: 107 (VH CDR2 of Ab10), and SEQ ID NO: 119 (VH CDR3 of Ab10), respectively.

In another specific embodiment, an antibody (e.g., human antibody) described herein comprises (i) a VL chain region comprising a VL CDR1, VL CDR2, and VL CDR3 having the amino acid sequences of SEQ ID NO: 12 (VL CDR1 of Ab11), SEQ ID NO: 24 (VL CDR2 of Ab11), and SEQ ID NO: 36 (VL CDR1 of Ab11), respectively; and (ii) a VH chain region comprising a VH CDR1, VH CDR2, and VH CDR3 having the amino acid sequences of SEQ ID NO: 96 (VH CDR1 of Ab11), SEQ ID NO: 108 (VH CDR2 of Ab11), and SEQ ID NO: 120 (VH CDR3 of Ab11), respectively.

In another specific embodiment, an antibody (e.g., human antibody) described herein comprises (i) a VL chain region comprising a VL CDR1, VL CDR2, and VL CDR3 having the amino acid sequences of SEQ ID NO: 13 (VL CDR1 of Ab12), SEQ ID NO: 25 (VL CDR2 of Ab12), and SEQ ID NO: 37 (VL CDR3 of Ab12), respectively; and (ii) a VH chain region comprising a VH CDR1, VH CDR2, and VH CDR3 having the amino acid sequences of SEQ ID NO: 97 (VH CDR1 of Ab12), SEQ ID NO: 109 (VH CDR2 of Ab12), and SEQ ID NO: 121 (VH CDR3 of Ab12), respectively.

In another specific embodiment, an antibody (e.g., human antibody) described herein comprises (i) a VL chain region comprising a VL CDR1, VL CDR2, and VL CDR3 having the amino acid sequences of SEQ ID NO: 330 (VL CDR1 of Ab21), SEQ ID NO: 331 (VL CDR2 of Ab21), and SEQ ID NO: 332 (VL CDR3 of Ab21), respectively; and (ii) a VH chain region comprising a VH CDR1, VH CDR2, and VH CDR3 having the amino acid sequences of SEQ ID NO: 337 (VH CDR1 of Ab21), SEQ ID NO: 338 (VH CDR2 of Ab21), and SEQ ID NO: 339 (VH CDR3 of Ab21), respectively.

In particular embodiments, an antibody described herein comprises (i) a VL chain region comprising a VL CDR1 having the amino acid sequence of any one of SEQ ID NOs: 2-13 and 330, a VL CDR2 having the amino acid sequence of any one of SEQ ID NOs: 14-25 and 331, and a VL CDR3 having the amino acid sequence of any one of SEQ ID NOs: 26-37 and 332; and (ii) a VH chain region having the amino acid sequence of any one of SEQ ID NO: 188 (Ab1), SEQ ID NO: 189 (Ab2), SEQ ID NO: 190 (Ab3), SEQ ID NO: 191 (Ab4), SEQ ID NO: 192 (Ab5), SEQ ID NO: 193 (Ab6), SEQ ID NO: 194 (Ab7), SEQ ID NO: 195 (Ab8), SEQ ID NO: 196 (Ab9), SEQ ID NO: 197 (Ab10), SEQ ID NO: 198 (Ab11), SEQ ID NO: 199 (Ab12), and SEQ ID NO: 329; wherein the antibody immunospecifically binds to a KIT polypeptide, e.g., a human KIT polypeptide, for example, a D4/D5 region of KIT (e.g., human KIT), for example SEQ ID NO: 171.

In particular embodiments, an antibody described herein comprises (i) a VL chain region comprising a VL CDR1 having the amino acid sequence of any one of SEQ ID NOs: 200-207, a VL CDR2 having the amino acid sequence of any one of SEQ ID NOs: 208-215, and a VL CDR3 having the amino acid sequence of any one of SEQ ID NOs: 216-223; and (ii) a VH chain region having the amino acid sequence of any one of SEQ ID NOs: 320-327 (Ab13-Ab20); wherein the antibody immunospecifically binds to a KIT polypeptide, e.g., a human KIT polypeptide, for example, a D4/D5 region of KIT (e.g., human KIT), for example SEQ ID NO: 171.

In a particular embodiment, an antibody described herein comprises (i) a VL chain region comprising a VL CDR1 having the amino acid sequence of any one of SEQ ID NOs: 2-13, a VL CDR2 having the amino acid sequence of any one of SEQ ID NOs: 14-25, and a VL CDR3 having the amino acid sequence of any one of SEQ ID NOs: 26-37; and (ii) a VH chain region having the amino acid sequence of any one of SEQ ID NO: 188 (Ab1), SEQ ID NO: 189 (Ab2), SEQ ID NO: 190 (Ab3), SEQ ID NO: 191 (Ab4), or SEQ ID NO: 192 (Ab5); wherein the antibody immunospecifically binds to a KIT polypeptide, e.g., a human KIT polypeptide, for example, a D4/D5 region of KIT (e.g., human KIT), for example SEQ ID NO: 171.

In another specific embodiment, an antibody (e.g., human antibody) described herein comprises (i) a VL chain region comprising a VL CDR1, VL CDR2, and VL CDR3 having the amino acid sequences of SEQ ID NO: 2 (VL CDR1 of Ab1), SEQ ID NO: 14 (VL CDR2 of Ab1), and SEQ ID NO: 26 (VL CDR3 of Ab1), respectively; and (ii) a VH chain region having the amino acid sequence of SEQ ID NO: 188 (Ab1).

In another specific embodiment, an antibody (e.g., human antibody) described herein comprises (i) a VL chain region comprising a VL CDR1, VL CDR2, and VL CDR3 having the amino acid sequences of SEQ ID NO: 3 (VL CDR1 of Ab2), SEQ ID NO: 15 (VL CDR2 of Ab2), and SEQ ID NO: 27 (VL CDR3 of Ab2), respectively; and (ii) a VH chain region having the amino acid sequence of SEQ ID NO: 189 (Ab2).

In another specific embodiment, an antibody (e.g., human antibody) described herein comprises (i) a VL chain region comprising a VL CDR1, VL CDR2, and VL CDR3 having the amino acid sequences of SEQ ID NO: 4 (VL CDR1 of Ab3), SEQ ID NO: 16 (VL CDR2 of Ab3), and SEQ ID NO: 28 (VL CDR3 of Ab3), respectively; and (ii) a VH chain region having the amino acid sequence of SEQ ID NO: 190 (Ab3).

In another specific embodiment, an antibody (e.g., human antibody) described herein comprises (i) a VL chain region comprising a VL CDR1, VL CDR2, and VL CDR3 having the amino acid sequences of SEQ ID NO: 5 (VL CDR1 of Ab4), SEQ ID NO: 17 (VL CDR2 of Ab4), and SEQ ID NO: 29 (VL CDR3 of Ab4), respectively; and (ii) a VH chain region having the amino acid sequence of SEQ ID NO: 191 (Ab4).

In another specific embodiment, an antibody (e.g., human antibody) described herein comprises (i) a VL chain region comprising a VL CDR1, VL CDR2, and VL CDR3 having the amino acid sequences of SEQ ID NO: 6 (VL CDR1 of Ab5), SEQ ID NO: 18 (VL CDR2 of Ab5), and SEQ ID NO: 30 (VL CDR3 of Ab5), respectively; and (ii) a VH chain region having the amino acid sequence of SEQ ID NO: 192 (Ab5).

In another specific embodiment, an antibody (e.g., human antibody) described herein comprises (i) a VL chain region comprising a VL CDR1, VL CDR2, and VL CDR3 having the amino acid sequences of SEQ ID NO: 7 (VL CDR1 of Ab6), SEQ ID NO: 19 (VL CDR2 of Ab6), and SEQ ID NO: 31 (VL CDR3 of Ab6), respectively; and (ii) a VH chain region having the amino acid sequence of SEQ ID NO: 193 (Ab6).

In another specific embodiment, an antibody (e.g., human antibody) described herein comprises (i) a VL chain region comprising a VL CDR1, VL CDR2, and VL CDR3 having the amino acid sequences of SEQ ID NO: 8 (VL CDR1 of Ab7), SEQ ID NO: 20 (VL CDR2 of Ab7), and SEQ ID NO: 32 (VL CDR3 of Ab7), respectively; and (ii) a VH chain region having the amino acid sequence of SEQ ID NO: 194 (Ab7).

In another specific embodiment, an antibody (e.g., human antibody) described herein comprises (i) a VL chain region comprising a VL CDR1, VL CDR2, and VL CDR3 having the amino acid sequences of SEQ ID NO: 9 (VL CDR1 of Ab8), SEQ ID NO: 21 (VL CDR2 of Ab8), and SEQ ID NO: 33 (VL CDR3 of Ab8), respectively; and (ii) a VH chain region having the amino acid sequence of SEQ ID NO: 195 (Ab8).

In another specific embodiment, an antibody (e.g., human antibody) described herein comprises (i) a VL chain region comprising a VL CDR1, VL CDR2, and VL CDR3 having the amino acid sequences of SEQ ID NO: 10 (VL CDR1 of Ab9), SEQ ID NO: 22 (VL CDR2 of Ab9), and SEQ ID NO: 34 (VL CDR3 of Ab9), respectively; and (ii) a VH chain region having the amino acid sequence of SEQ ID NO: 196 (Ab9).

In another specific embodiment, an antibody (e.g., human antibody) described herein comprises (i) a VL chain region comprising a VL CDR1, VL CDR2, and VL CDR3 having the amino acid sequences of SEQ ID NO: 11 (VL CDR1 of Ab10), SEQ ID NO: 23 (VL CDR2 of Ab10), and SEQ ID NO: 35 (VL CDR3 of Ab10), respectively; and (ii) a VH chain region having the amino acid sequence of SEQ ID NO: 197 (Ab10).

In another specific embodiment, an antibody (e.g., human antibody) described herein comprises (i) a VL chain region comprising a VL CDR1, VL CDR2, and VL CDR3 having the amino acid sequences of SEQ ID NO: 12 (VL CDR1 of Ab11), SEQ ID NO: 24 (VL CDR2 of Ab11), and SEQ ID NO: 36 (VL CDR3 of Ab11), respectively; and (ii) a VH chain region having the amino acid sequence of SEQ ID NO: 198 (Ab11).

In another specific embodiment, an antibody (e.g., human antibody) described herein comprises (i) a VL chain region comprising a VL CDR1, VL CDR2, and VL CDR3 having the amino acid sequences of SEQ ID NO: 13 (VL CDR1 of Ab12), SEQ ID NO: 25 (VL CDR2 of Ab12), and SEQ ID NO: 37 (VL CDR3 of Ab12), respectively; and (ii) a VH chain region having the amino acid sequence of SEQ ID NO: 199 (Ab12).

In another specific embodiment, an antibody (e.g., human antibody) described herein comprises (i) a VL chain region comprising a VL CDR1, VL CDR2, and VL CDR3 having the amino acid sequences of SEQ ID NO: 330 (VL CDR1 of Ab21), SEQ ID NO: 331 (VL CDR2 of Ab21), and SEQ ID NO: 332 (VL CDR3 of Ab21), respectively; and (ii) a VH chain region having the amino acid sequence of SEQ ID NO: 329 (Ab21).

In particular embodiments, an antibody described herein comprises (i) a VL chain region having the amino acid sequence of SEQ ID NO: 176 or 350 (Ab1), SEQ ID NO: 177 (Ab2), SEQ ID NO: 178 (Ab3), SEQ ID NO: 179 (Ab4), SEQ ID NO: 180(Ab5), SEQ ID NO: 181 (Ab6), SEQ ID NO: 182 (Ab7), SEQ ID NO: 183 (Ab8), SEQ ID NO: 184 (Ab9), SEQ ID NO: 185 (Ab10), SEQ ID NO: 186 (Ab11), SEQ ID NO: 187 (Ab12), or SEQ ID NO: 328 (AB21); and (ii) a VH chain region comprising a VH CDR1 having the amino acid sequence of any one of SEQ ID NOs: 86-97 and 337, a VH CDR2 having the amino acid sequence of any one of SEQ ID NOs: 98-109 and 338, and a VH CDR3 having the amino acid sequence of any one of SEQ ID NOs: 110-121 and 339. In particular embodiments, an antibody described herein comprises (i) a VL chain region having the amino acid sequence of any one of SEQ ID NOs: 312-319 (Ab13-Ab20); and (ii) a VH chain region comprising a VH CDR1 having the amino acid sequence of any one of SEQ ID NOs: 256-263, a VH CDR2 having the amino acid sequence of any one of SEQ ID NOs: 264-271, and a VH CDR3 having the amino acid sequence of any one of SEQ ID NOs: 272-279.

In another specific embodiment, an antibody (e.g., human antibody) described herein comprises (i) a VL chain region having the amino acid sequence of SEQ ID NO: 176 or 350 (Ab1); and (ii) a VH chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively. In a specific embodiment, the VL chain region has the amino acid sequence of SEQ ID NO: 176 (Ab1) starting at the second amino acid sequence of SEQ ID NO: 176. In a specific embodiment, the VL chain region has the amino acid sequence of SEQ ID NO: 176 (Ab1) starting at the second amino acid sequence of SEQ ID NO: 176 and/or the VL chain region further comprises an R (Arg) amino acid residue at the C-terminus.

In another specific embodiment, an antibody (e.g., human antibody) described herein comprises (i) a VL chain region having the amino acid sequence of SEQ ID NO: 963 (Ab1); and (ii) a VH chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively.

In another specific embodiment, an antibody (e.g., human antibody) described herein comprises (i) a VL chain region having the amino acid sequence of SEQ ID NO: 177 (Ab2); and (ii) a VH chain region comprising a VH CDR1, VH CDR2, and VH CDR3 having the amino acid sequences of SEQ ID NO: 87 (VH CDR1 of Ab2), SEQ ID NO: 99 (VH CDR2 of Ab2), and SEQ ID NO: 111 (VH CDR3 of Ab2), respectively. In a specific embodiment, the VL chain region has the amino acid sequence of SEQ ID NO: 177 (Ab2) starting at the second amino acid sequence of SEQ ID NO: 177. In a specific embodiment, the VL chain region starts at the second amino acid position and/or further comprises an R (Arg) amino acid residue at the C-terminus.

In another specific embodiment, an antibody (e.g., human antibody) described herein comprises (i) a VL chain region having the amino acid sequence of SEQ ID NO: 178 (Ab3); and (ii) a VH chain region comprising a VH CDR1, VH CDR2, and VH CDR3 having the amino acid sequences of SEQ ID NO: 88 (VH CDR1 of Ab3), SEQ ID NO: 100 (VH CDR2 of Ab3), and SEQ ID NO: 112 (VH CDR3 of Ab3), respectively. In a specific embodiment, the VL chain region has the amino acid sequence of SEQ ID NO: 178 (Ab3) starting at the second amino acid sequence of SEQ ID NO: 178. In a specific embodiment, the VL chain region starts at the second amino acid position and/or further comprises an R (Arg) amino acid residue at the C-terminus.

In another specific embodiment, an antibody (e.g., human antibody) described herein comprises (i) a VL chain region having the amino acid sequence of SEQ ID NO: 179 (Ab4); and (ii) a VH chain region comprising a VH CDR1, VH CDR2, and VH CDR3 having the amino acid sequences of SEQ ID NO: 89 (VH CDR1 of Ab4), SEQ ID NO: 101 (VH CDR2 of Ab4), and SEQ ID NO: 113 (VH CDR3 of Ab4), respectively. In a specific embodiment, the VL chain region has the amino acid sequence of SEQ ID NO: 179 (Ab4) starting at the second amino acid sequence of SEQ ID NO: 179. In a specific embodiment, the VL chain region starts at the second amino acid position and/or further comprises an R (Arg) amino acid residue at the C-terminus.

In another specific embodiment, an antibody (e.g., human antibody) described herein comprises (i) a VL chain region having the amino acid sequence of SEQ ID NO: 180 (Ab5); and (ii) a VH chain region comprising a VH CDR1, VH CDR2, and VH CDR3 having the amino acid sequences of SEQ ID NO: 90 (VH CDR1 of Ab5), SEQ ID NO: 102 (VH CDR2 of Ab5), and SEQ ID NO: 114 (VH CDR3 of Ab5), respectively. In a specific embodiment, the VL chain region has the amino acid sequence of SEQ ID NO: 180 (Ab5) starting at the second amino acid sequence of SEQ ID NO: 180. In a specific embodiment, the VL chain region starts at the second amino acid position and/or further comprises an R (Arg) amino acid residue at the C-terminus.

In another specific embodiment, an antibody (e.g., human antibody) described herein comprises (i) a VL chain region having the amino acid sequence of SEQ ID NO: 181 (Ab6); and (ii) a VH chain region comprising a VH CDR1, VH CDR2, and VH CDR3 having the amino acid sequences of SEQ ID NO: 91 (VH CDR1 of Ab6), SEQ ID NO: 103 (VH CDR2 of Ab6), and SEQ ID NO: 115 (VH CDR3 of Ab6), respectively. In a specific embodiment, the VL chain region has the amino acid sequence of SEQ ID NO: 181 (Ab6) starting at the second amino acid sequence of SEQ ID NO: 181. In a specific embodiment, the VL chain region starts at the second amino acid position and/or further comprises an R (Arg) amino acid residue at the C-terminus.

In another specific embodiment, an antibody (e.g., human antibody) described herein comprises (i) a VL chain region having the amino acid sequence of SEQ ID NO: 182 (Ab7); and (ii) a VH chain region comprising a VH CDR1, VH CDR2, and VH CDR3 having the amino acid sequences of SEQ ID NO: 92 (VH CDR1 of Ab7), SEQ ID NO: 104 (VH CDR2 of Ab7), and SEQ ID NO: 116 (VH CDR3 of Ab7), respectively. In a specific embodiment, the VL chain region has the amino acid sequence of SEQ ID NO: 182 (Ab7) starting at the second amino acid sequence of SEQ ID NO: 182. In a specific embodiment, the VL chain region starts at the second amino acid position and/or further comprises an R (Arg) amino acid residue at the C-terminus.

In another specific embodiment, an antibody (e.g., human antibody) described herein comprises (i) a VL chain region having the amino acid sequence of SEQ ID NO: 183 (Ab8); and (ii) a VH chain region comprising a VH CDR1, VH CDR2, and VH CDR3 having the amino acid sequences of SEQ ID NO: 93 (VH CDR1 of Ab8), SEQ ID NO: 105 (VH CDR2 of Ab8), and SEQ ID NO: 117 (VH CDR3 of Ab8), respectively. In a specific embodiment, the VL chain region has the amino acid sequence of SEQ ID NO: 183 (Ab8) starting at the second amino acid sequence of SEQ ID NO: 183. In a specific embodiment, the VL chain region starts at the second amino acid position and/or further comprises an R (Arg) amino acid residue at the C-terminus.

In another specific embodiment, an antibody (e.g., human antibody) described herein comprises (i) a VL chain region having the amino acid sequence of SEQ ID NO: 184 (Ab9); and (ii) a VH chain region comprising a VH CDR1, VH CDR2, and VH CDR3 having the amino acid sequences of SEQ ID NO: 94 (VH CDR1 of Ab9), SEQ ID NO: 106 (VH CDR2 of Ab9), and SEQ ID NO: 118 (VH CDR3 of Ab9), respectively. In a specific embodiment, the VL chain region has the amino acid sequence of SEQ ID NO: 184 (Ab9) starting at the second amino acid sequence of SEQ ID NO: 184. In a specific embodiment, the VL chain region starts at the second amino acid position and/or further comprises an R (Arg) amino acid residue at the C-terminus.

In another specific embodiment, an antibody (e.g., human antibody) described herein comprises (i) a VL chain region having the amino acid sequence of SEQ ID NO: 185 (Ab10); and (ii) a VH chain region comprising a VH CDR1, VH CDR2, and VH CDR3 having the amino acid sequences of SEQ ID NO: 95 (VH CDR1 of Ab10), SEQ ID NO: 107 (VH CDR2 of Ab10), and SEQ ID NO: 119 (VH CDR3 of Ab10), respectively. In a specific embodiment, the VL chain region has the amino acid sequence of SEQ ID NO: 185 (Ab10) starting at the second amino acid sequence of SEQ ID NO: 185. In a specific embodiment, the VL chain region starts at the second amino acid position and/or further comprises an R (Arg) amino acid residue at the C-terminus.

In another specific embodiment, an antibody (e.g., human antibody) described herein comprises (i) a VL chain region having the amino acid sequence of SEQ ID NO: 186 (Ab11); and (ii) a VH chain region comprising a VH CDR1, VH CDR2, and VH CDR3 having the amino acid sequences of SEQ ID NO: 96 (VH CDR1 of Ab11), SEQ ID NO: 108 (VH CDR2 of Ab11), and SEQ ID NO: 120 (VH CDR3 of Ab11), respectively. In a specific embodiment, the VL chain region has the amino acid sequence of SEQ ID NO: 186 (Ab11) starting at the second amino acid sequence of SEQ ID NO: 186. In a specific embodiment, the VL chain region starts at the second amino acid position and/or further comprises an R (Arg) amino acid residue at the C-terminus.

In another specific embodiment, an antibody (e.g., human antibody) described herein comprises (i) a VL chain region having the amino acid sequence of SEQ ID NO: 187 (Ab12); and (ii) a VH chain region comprising a VH CDR1, VH CDR2, and VH CDR3 having the amino acid sequences of SEQ ID NO: 97 (VH CDR1 of Ab12), SEQ ID NO: 109 (VH CDR2 of Ab12), and SEQ ID NO: 121 (VH CDR3 of Ab12), respectively. In a specific embodiment, the VL chain region has the amino acid sequence of SEQ ID NO: 187 (Ab12) starting at the second amino acid sequence of SEQ ID NO: 187. In a specific embodiment, the VL chain region starts at the second amino acid position and/or further comprises an R (Arg) amino acid residue at the C-terminus.

In another specific embodiment, an antibody (e.g., human antibody) described herein comprises (i) a VL chain region having the amino acid sequence of SEQ ID NO: 328 (Ab21); and (ii) a VH chain region comprising a VH CDR1, VH CDR2, and VH CDR3 having the amino acid sequences of SEQ ID NO: 337 (VH CDR1 of Ab21), SEQ ID NO: 338 (VH CDR2 of Ab21), and SEQ ID NO: 339 (VH CDR3 of Ab21), respectively. In another specific embodiment, an antibody (e.g., human antibody) described herein comprises (i) a VL chain region having the amino acid sequence of SEQ ID NO: 964 (Ab21); and (ii) a VH chain region comprising a VH CDR1, VH CDR2, and VH CDR3 having the amino acid sequences of SEQ ID NO: 337 (VH CDR1 of Ab21), SEQ ID NO: 338 (VH CDR2 of Ab21), and SEQ ID NO: 339 (VH CDR3 of Ab21), respectively.

In specific aspects, provided herein is an antibody which competitively blocks (e.g., in a dose dependent manner), antibodies comprising the amino acid sequences described herein for specific binding to a KIT polypeptide (e.g., a D4/D5 region of KIT, for example human KIT), as determined using assays known to one of skill in the art or described herein (e.g., ELISA competitive assays). In particular embodiments, such competitively blocking antibody inhibits one or more KIT activities.

In a specific embodiment, an antibody described herein is one that is competitively blocked (e.g., in a dose dependent manner) by an antibody comprising a VL chain region having the amino acid sequence of SEQ ID NO: 176, 350, or 963 (VL domain of Ab1) and a VH chain region having the amino acid sequence of SEQ ID NO: 188 (VH domain of Ab1), for specific binding to a KIT polypeptide (e.g., a D4/D5 region of KIT, for example human KIT). In a specific embodiment, the VL chain region has the amino acid sequence of SEQ ID NO: 176 (Ab1) starting at the second amino acid sequence of SEQ ID NO: 176. In a specific embodiment, such an Ab1 antibody is competitively blocked (e.g., in a dose dependent manner) by antibody Ab21 for specific binding to a KIT polypeptide (e.g., a D4/D5 region of KIT, for example human KIT. In another specific embodiment, such an Ab1 antibody is competitively blocked (e.g., in a dose dependent manner) by an antibody comprising a VL domain having the amino acid sequence of SEQ ID NO: 328 or 964 (VL domain of antibody Ab21) and/or a VH domain having the amino acid sequence of SEQ ID NO: 329 (VH domain of antibody Ab21). In yet another embodiment, such an Ab1 antibody is competitively blocked (e.g., in a dose dependent manner) by an antibody comprising the VH CDRs (e.g., SEQ ID NOs: 337, 338, and/or 339) and VL CDRs (e.g., SEQ ID NOs: 330, 331, and/or 332) of antibody Ab21. In a specific embodiment, antibody Ab21 (e.g., antibody comprising SEQ ID NOs: 328 (or 964) and 329) is competitively blocked (e.g., in a dose dependent manner) by antibody Ab1 for specific binding to a KIT polypeptide (e.g., a D4/D5 region of KIT, for example human KIT. In another specific embodiment, such an Ab21 antibody is competitively blocked (e.g., in a dose dependent manner) by an antibody comprising a VL domain having the amino acid sequence of SEQ ID NO: 176, 350, or 963 (VL domain of antibody Ab1) and/or a VH domain having the amino acid sequence of SEQ ID NO: 188 (VH domain of antibody Ab1). In yet another specific embodiment, such an Ab21 antibody is competitively blocked (e.g., in a dose dependent manner) by an antibody comprising the VH CDRs (e.g., SEQ ID NOs: 86, 98, and/or 110) and VL CDRs (e.g., SEQ ID NOs: 2, 14, and/or 26) of antibody Ab1.

In another specific embodiment, an antibody described herein is one that is competitively blocked (e.g., in a dose dependent manner) by an antibody comprising a VL chain region having the amino acid sequence of SEQ ID NO: 177 (VL domain of Ab2) and a VH chain region having the amino acid sequence of SEQ ID NO: 189 (VH domain of Ab2), for specific binding to a KIT polypeptide (e.g., a D4/D5 region of KIT, for example human KIT).

In another specific embodiment, an antibody described herein is one that is competitively blocked (e.g., in a dose dependent manner) by an antibody comprising a VL chain region having the amino acid sequence of SEQ ID NO: 178 (VL domain of Ab3), optionally starting at the second amino acid position and/or further comprising an R (Arg) at the C-terminus, and a VH chain region having the amino acid sequence of SEQ ID NO: 190 (VH domain of Ab3), for specific binding to a KIT polypeptide (e.g., a D4/D5 region of KIT, for example human KIT).

In another specific embodiment, an antibody described herein is one that is competitively blocked (e.g., in a dose dependent manner) by an antibody comprising a VL chain region having the amino acid sequence of SEQ ID NO: 179 (VL domain of Ab4), optionally starting at the second amino acid position and/or further comprising an R (Arg) at the C-terminus, and a VH chain region having the amino acid sequence of SEQ ID NO: 191 (VH domain of Ab4), for specific binding to a KIT polypeptide (e.g., a D4/D5 region of KIT, for example human KIT).

In another specific embodiment, an antibody described herein is one that is competitively blocked (e.g., in a dose dependent manner) by an antibody comprising a VL chain region having the amino acid sequence of SEQ ID NO: 180 (VL domain of Ab5), optionally starting at the second amino acid position and/or further comprising an R (Arg) at the C-terminus, and a VH chain region having the amino acid sequence of SEQ ID NO: 192 (VH domain of Ab5), for specific binding to a KIT polypeptide (e.g., a D4/D5 region of KIT, for example human KIT).

In another specific embodiment, an antibody described herein is one that is competitively blocked (e.g., in a dose dependent manner) by an antibody comprising a VL chain region having the amino acid sequence of SEQ ID NO: 181 (VL domain of Ab6), optionally starting at the second amino acid position and/or further comprising an R (Arg) at the C-terminus, and a VH chain region having the amino acid sequence of SEQ ID NO: 193 (VH domain of Ab6), for specific binding to a KIT polypeptide (e.g., a D4/D5 region of KIT, for example human KIT).

In another specific embodiment, an antibody described herein is one that is competitively blocked (e.g., in a dose dependent manner) by an antibody comprising a VL chain region having the amino acid sequence of SEQ ID NO: 182 (VL domain of Ab7), optionally starting at the second amino acid position and/or further comprising an R (Arg) at the C-terminus, and a VH chain region having the amino acid sequence of SEQ ID NO: 194 (VH domain of Ab7), for specific binding to a KIT polypeptide (e.g., a D4/D5 region of KIT, for example human KIT).

In another specific embodiment, an antibody described herein is one that is competitively blocked (e.g., in a dose dependent manner) by an antibody comprising a VL chain region having the amino acid sequence of SEQ ID NO: 183 (VL domain of Ab8), optionally starting at the second amino acid position and/or further comprising an R (Arg) at the C-terminus, and a VH chain region having the amino acid sequence of SEQ ID NO: 195 (VH domain of Ab8), for specific binding to a KIT polypeptide (e.g., a D4/D5 region of KIT, for example human KIT).

In another specific embodiment, an antibody described herein is one that is competitively blocked (e.g., in a dose dependent manner) by an antibody comprising a VL chain region having the amino acid sequence of SEQ ID NO: 184 (VL domain of Ab9), optionally starting at the second amino acid position and/or further comprising an R (Arg) at the C-terminus, and a VH chain region having the amino acid sequence of SEQ ID NO: 196 (VH domain of Ab9), for specific binding to a KIT polypeptide (e.g., a D4/D5 region of KIT, for example human KIT).

In another specific embodiment, an antibody described herein is one that is competitively blocked (e.g., in a dose dependent manner) by an antibody comprising a VL chain region having the amino acid sequence of SEQ ID NO: 185 (VL domain of Ab10), optionally starting at the second amino acid position and/or further comprising an R (Arg) at the C-terminus, and a VH chain region having the amino acid sequence of SEQ ID NO: 197 (VH domain of Ab10), for specific binding to a KIT polypeptide (e.g., a D4/D5 region of KIT, for example human KIT).

In another specific embodiment, an antibody described herein is one that is competitively blocked (e.g., in a dose dependent manner) by an antibody comprising a VL chain region having the amino acid sequence of SEQ ID NO: 186 (VL domain of Ab11), optionally starting at the second amino acid position and/or further comprising an R (Arg) at the C-terminus, and a VH chain region having the amino acid sequence of SEQ ID NO: 198 (VH domain of Ab11), for specific binding to a KIT polypeptide (e.g., a D4/D5 region of KIT, for example human KIT).

In another specific embodiment, an antibody described herein is one that is competitively blocked (e.g., in a dose dependent manner) by an antibody comprising a VL chain region having the amino acid sequence of SEQ ID NO: 187 (VL domain of Ab12), optionally starting at the second amino acid position and/or further comprising an R (Arg) at the C-terminus, and a VH chain region having the amino acid sequence of SEQ ID NO: 199 (VH domain of Ab12), for specific binding to a KIT polypeptide (e.g., a D4/D5 region of KIT, for example human KIT).

In a specific embodiment, an antibody described herein is one that is competitively blocked (e.g., in a dose dependent manner) by an antibody comprising a VL chain region having the amino acid sequence of SEQ ID NO: 328 (VL domain of Ab21), optionally comprising an R (Arg) amino acid residue at the C-terminus, and a VH chain region having the amino acid sequence of SEQ ID NO: 329 (VH domain of Ab21), for specific binding to a KIT polypeptide (e.g., a D4/D5 region of KIT, for example human KIT). In a specific embodiment, an antibody described herein is one that is competitively blocked (e.g., in a dose dependent manner) by an antibody comprising a VL chain region having the amino acid sequence of SEQ ID NO: 964 (VL domain of Ab21) and a VH chain region having the amino acid sequence of SEQ ID NO: 329 (VH domain of Ab21), for specific binding to a KIT polypeptide (e.g., a D4/D5 region of KIT, for example human KIT).

In a specific embodiment, an antibody described herein is one that is competitively blocked (e.g., in a dose dependent manner) by an antibody comprising a VL chain region having the amino acid sequence of any one of SEQ ID NOs: 312-319 (VL domain of Ab13-20), optionally starting at the second amino acid position and/or comprising an R (Arg) amino acid residue at the C-terminus, and a VH chain region having the amino acid sequence of any one of SEQ ID NO: 320-327 (VH domain of Ab13-Ab20), for specific binding to a KIT polypeptide (e.g., a D4/D5 region of KIT, for example human KIT).

Competition binding assays can be used to determine whether an antibody is competitively blocked (e.g., in a dose dependent manner) by another antibody for specific binding to an antigen (e.g., KIT, for example a D4/D5 region of KIT, e.g., human KIT). Such methods determine if antibodies are able to block each other's binding to an antigen, and work for both conformational and linear epitopes. Competition binding assays can also be used to determine whether two antibodies have similar binding specificity for an epitope. For example, an antibody binds essentially the same epitope as a reference antibody, when the two antibodies recognize identical or sterically overlapping epitopes in competition binding assays such as competition ELISA assays, which can be configured in all number of different formats, using either labeled antigen or labeled antibody. The competition binding assay can be configured in a large number of different formats using either labeled antigen or labeled antibody. In the most common version of this assay, the antigen is immobilized on a 96-well plate. The ability of unlabeled antibodies to block the binding of labeled antibodies to the antigen is then measured using radioactive or enzyme labels. For further details see, for example, Wagener et al., J. Immunol., 1983, 130:2308-2315; Wagener et al., J. Immunol. Methods, 1984, 68:269-274; Kuroki et al., Cancer Res., 1990, 50:4872-4879; Kuroki et al., Immunol. Invest., 1992, 21:523-538; Kuroki et al., Hybridoma, 1992, 11:391-407, and Using Antibodies: A Laboratory Manual, Ed Harlow and David Lane editors (Cold Springs Harbor Laboratory Press, Cold Springs Harbor, N.Y., 1999), pp. 386-389. In a particular embodiment, an antibody can be tested in competition binding assays with an antibody described herein, e.g., Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, or Ab21. In another particular embodiment, an antibody can be tested in competition binding assays with an antibody described herein, e.g., a monoclonal antibody comprising the VL chain region and VH chain region of Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, or Ab21. In another particular embodiment, an antibody can be tested in competition binding assays with an antibody described herein, e.g., a monoclonal antibody comprising the VL chain region and VH chain region of Ab1 or Ab21. In another particular embodiment, an antibody can be tested in competition binding assays with an antibody described herein, e.g., an IgG1 human monoclonal antibody comprising the VH CDRs and VL CDRs of Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, or Ab21. In another particular embodiment, an antibody can be tested in competition binding assays with an antibody described herein, e.g., an IgG1 human monoclonal antibody comprising the VH CDRs and VL CDRs of Ab13, Ab14, Ab15, Ab16, Ab17, Ab18, Ab19, Ab20, or Ab21. Ina specific embodiment, a competition binding assay is carried out as described in section 6.6 below.

In a specific embodiment, an antibody described herein immunospecifically binds to the same epitope as an epitope to which antibody Ab1 binds. In a specific embodiment, an antibody described herein immunospecifically binds to the same epitope as an epitope to which antibody Ab21 binds. In a specific embodiment, an antibody described herein immunospecifically binds to the same epitope as an epitope to which antibody Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, or Ab21 binds. In a specific embodiment, antibody Ab1 binds to the same epitope as antibody Ab21.

Table 1A, below, presents the VL CDR (in particular, VL CDR1, VL CDR2, and VL CDR3) amino acid sequences of Ab1-Ab12. Table 1B, below, presents the VL CDR (in particular, VL CDR1, VL CDR2, and VL CDR3) amino acid sequences of Ab13-Ab21. Table 1C, below, presents the VL CDR (in particular, VL CDR1, VL CDR2, and VL CDR3) amino acid sequences of Ab24-Ab36 and Ab38-Ab192. Table 2A, below, presents the VL framework (FR) amino acid sequences (in particular, VL FR1, VL FR2, VL FR3, and VL FR4 sequences) of Ab1-Ab12. Table 2B, below, presents the VL framework (FR) amino acid sequences (in particular, VL FR1, VL FR2, VL FR3, and VL FR4 sequences) of Ab13-Ab21. Table 3A, below, presents the VH CDR (in particular, VH CDR1, VH CDR2, and VH CDR3) amino acid sequences of Ab1-Ab12. Table 3B, below, presents the VH CDR (in particular, VH CDR1, VH CDR2, and VH CDR3) amino acid sequences of Ab13-Ab21. Table 3C, below, presents the VH CDR (in particular, VH CDR1, VH CDR2, and VH CDR3) amino acid sequences of Ab24-Ab36 and Ab38-Ab192. Table 4A, below, presents the VH FR amino acid sequences (in particular, VH FR1, VH FR2, VH FR3, and VH FR4 sequences) of Ab1-Ab12. Table 4B, below, presents the VH FR amino acid sequences (in particular, VH FR1, VH FR2, VH FR3, and VH FR4 sequences) of Ab13-Ab21.

In specific embodiments, an antibody described herein immunospecifically binds to a D4/D5 region of human KIT (e.g., SEQ ID NO: 171), wherein said antibody comprises one or more VL CDR sequences selected from those in Tables 1A, 1B, and 1C. In specific embodiments, an antibody described herein immunospecifically binds to a D4/D5 region of human KIT (e.g., SEQ ID NO: 171), wherein said antibody comprises one or more VL FR sequences selected from those in Tables 2A and 2B. In specific embodiments, an antibody described herein immunospecifically binds to a D4/D5 region of human KIT (e.g., SEQ ID NO: 171), wherein said antibody comprises one or more VH CDR sequences selected from those in Tables 3A, 3B, and 3C. In specific embodiments, an antibody described herein immunospecifically binds to a D4/D5 region of human KIT (e.g., SEQ ID NO: 171), wherein said antibody comprises one or more VH FR sequences selected from those in Table 4A and 4B. In particular embodiments, such antibody is capable of one or more KIT activities, e.g., inhibiting tyrosine phosphorylation of the cytoplasmic domain of KIT.

In a specific embodiment, an antibody described herein immunospecifically binds to a D4/D5 region of human KIT (e.g., SEQ ID NO: 171), wherein said antibody comprises (i) one, two, and/or three VL CDRs selected from those in Tables 1A, 1B, and 1C, and (ii) one, two, and/or three VH CDRs selected from those in Tables 3A, 3B, and 3C.

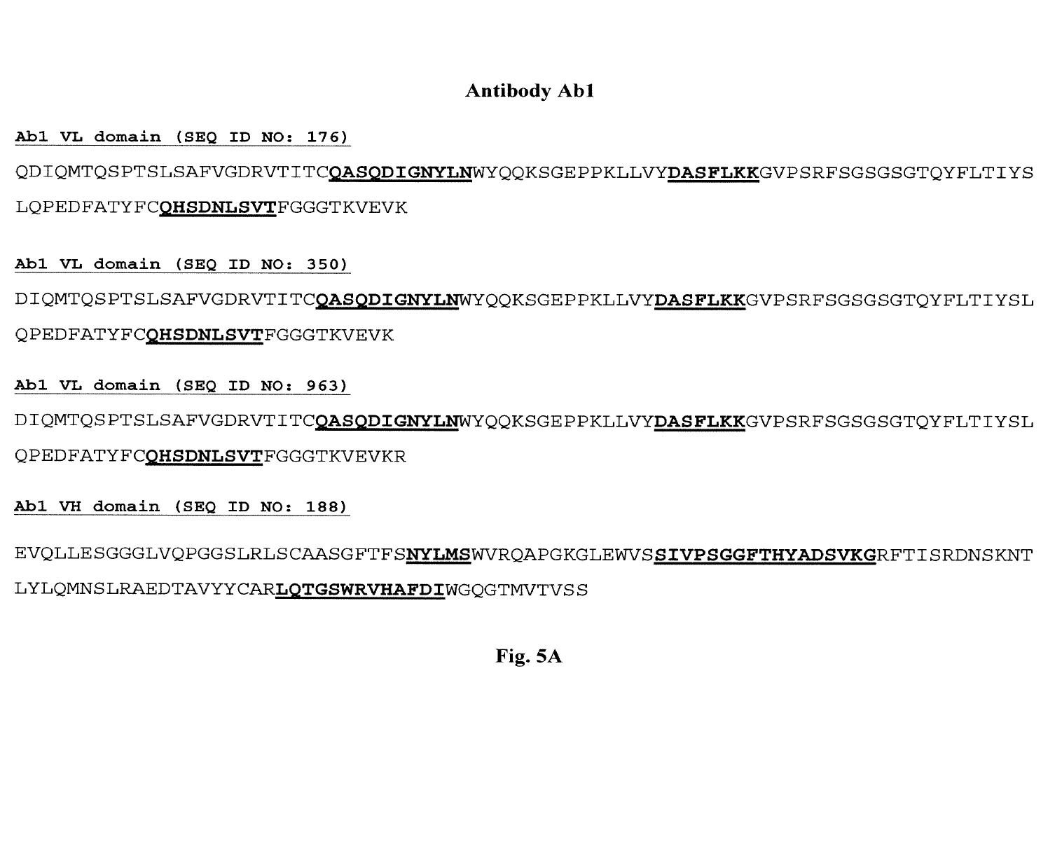

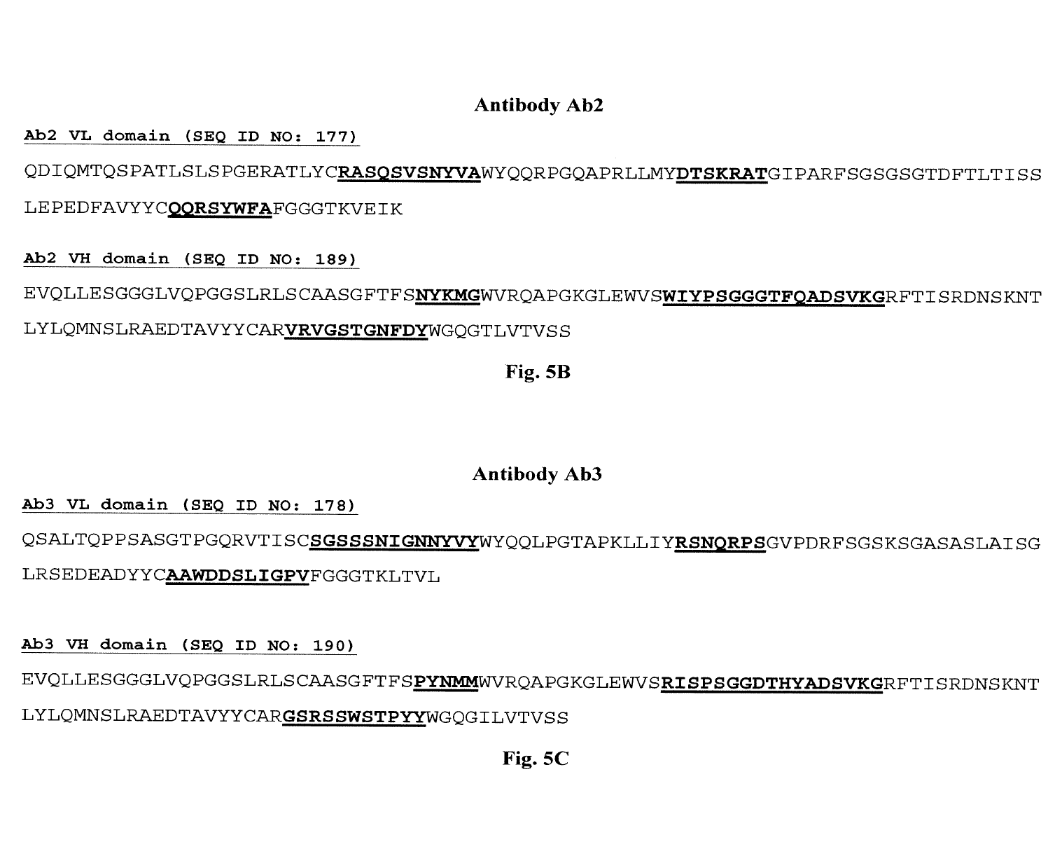

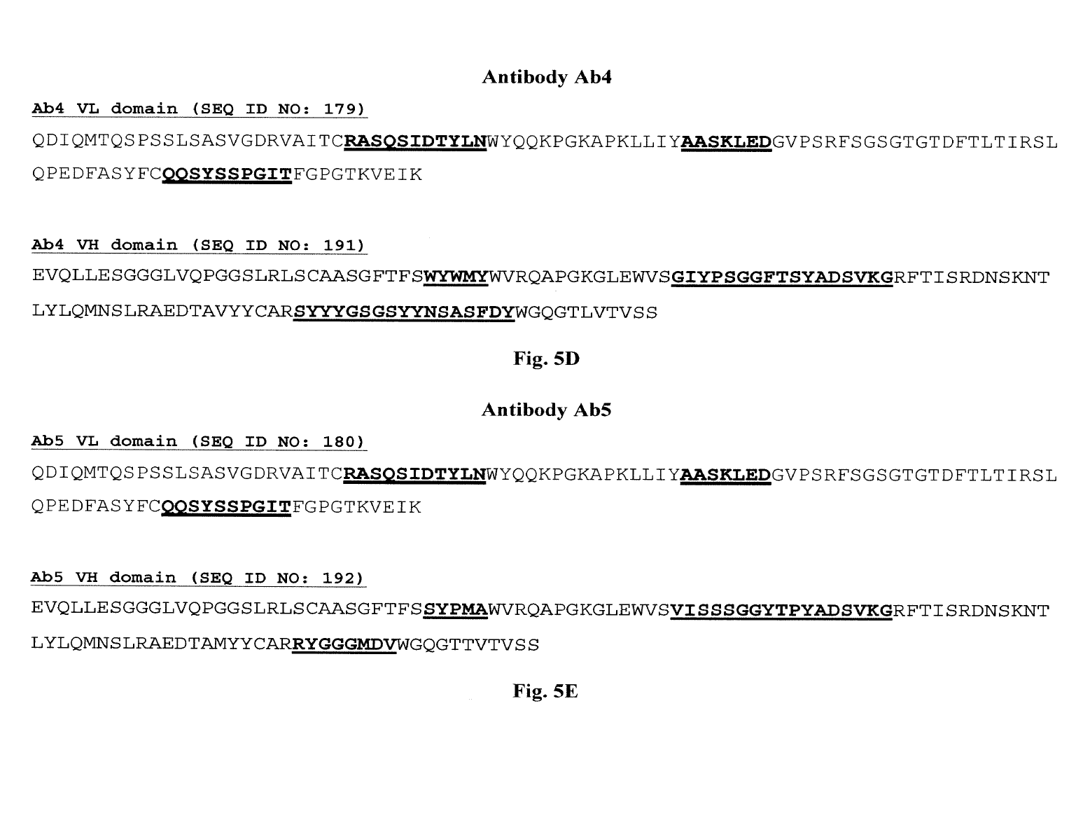

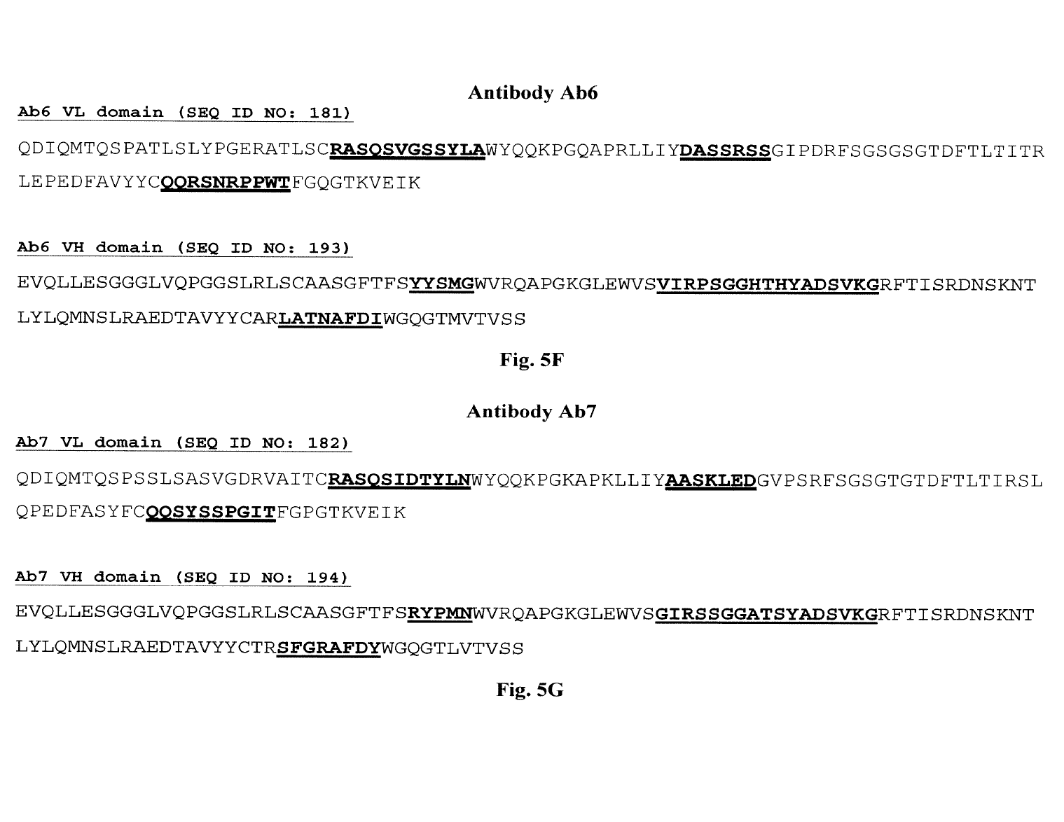

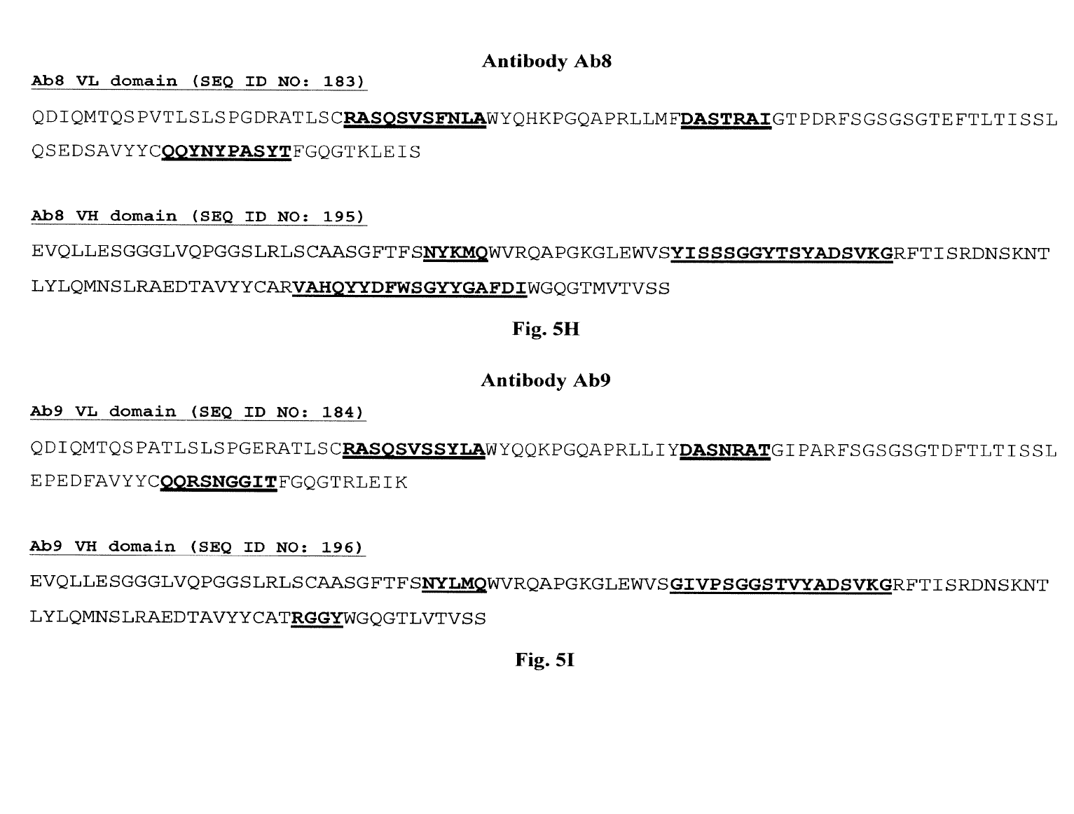

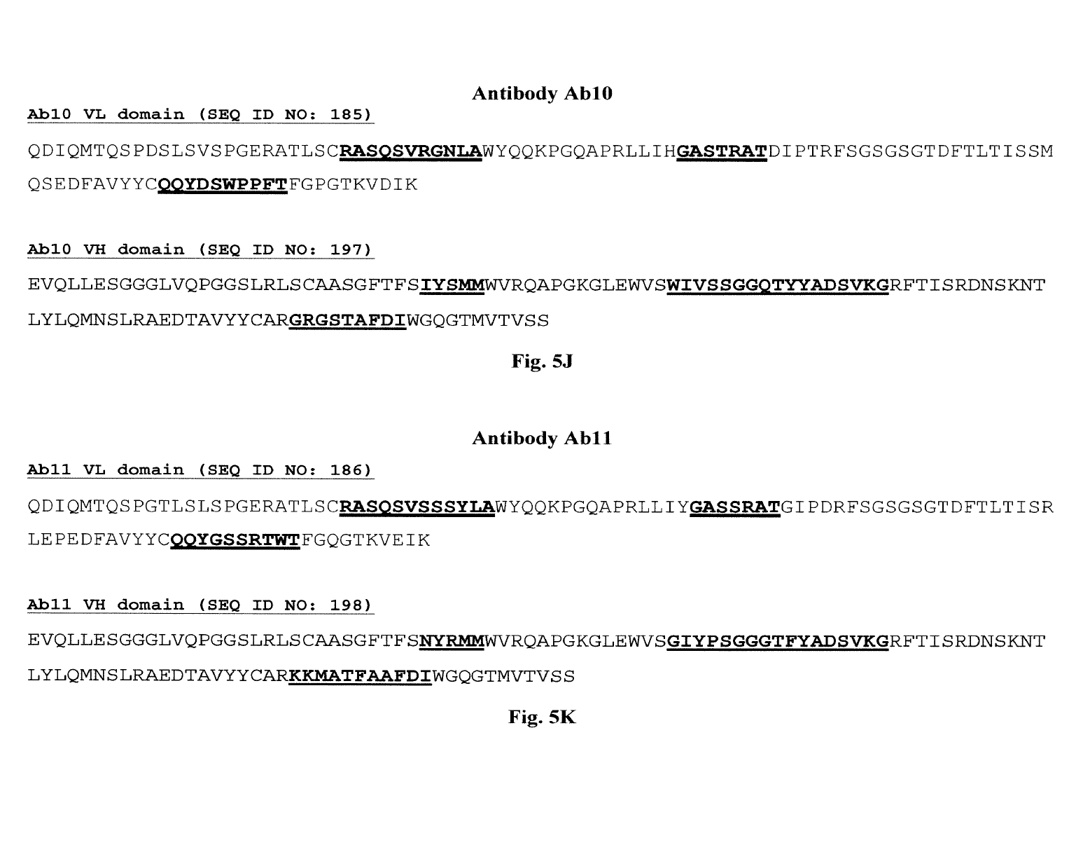

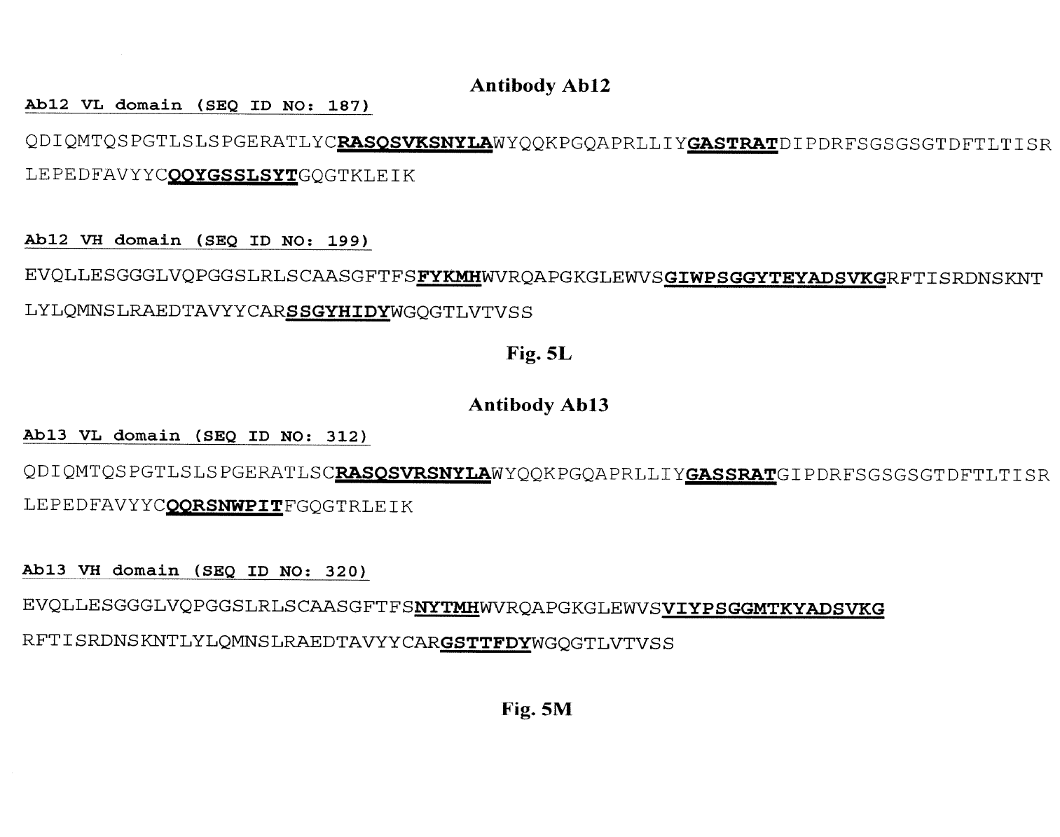

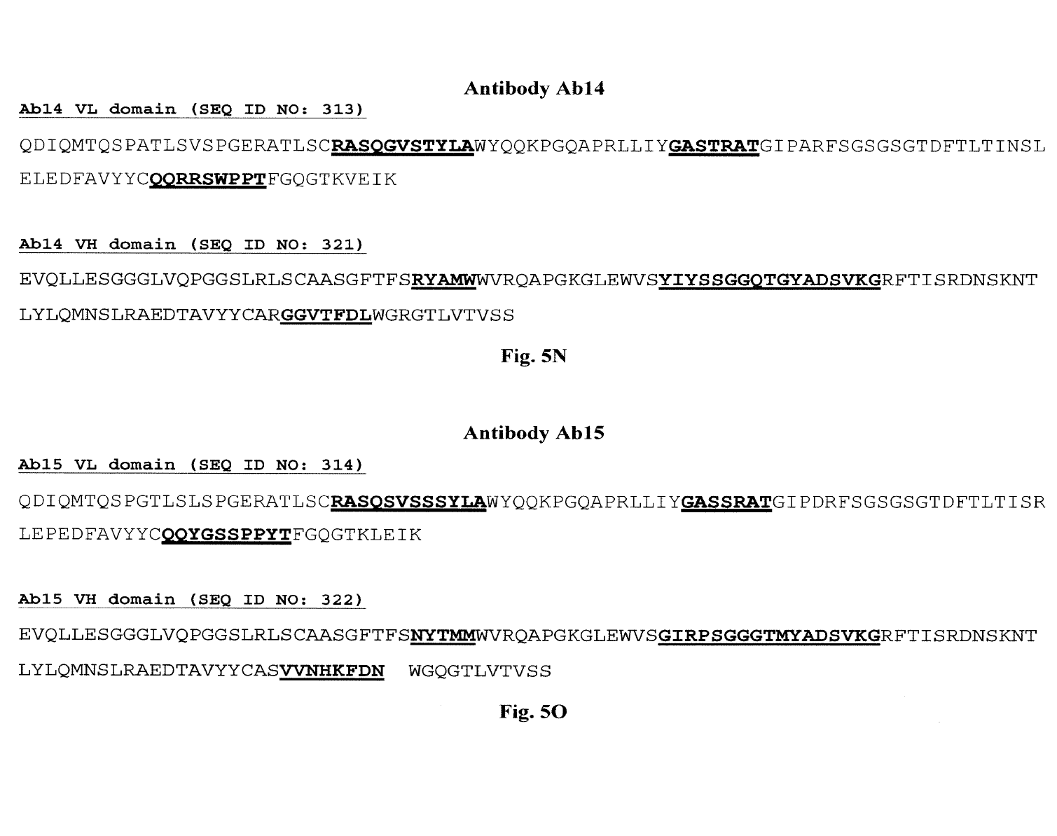

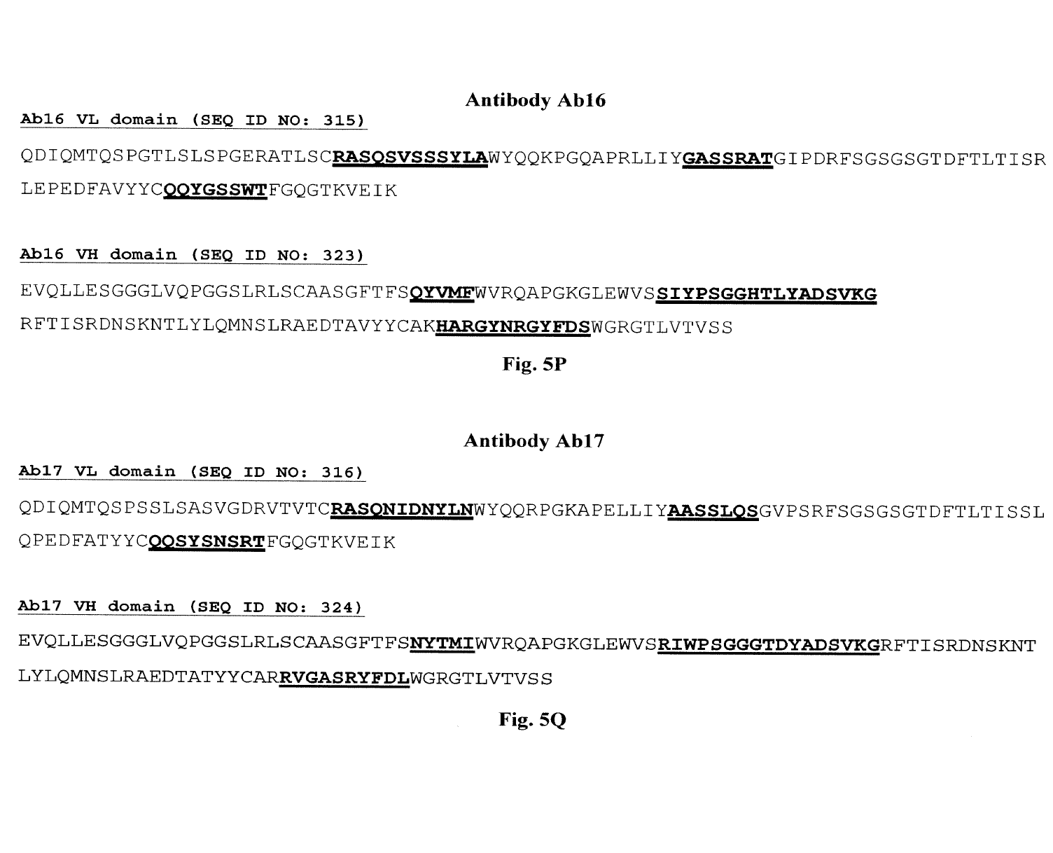

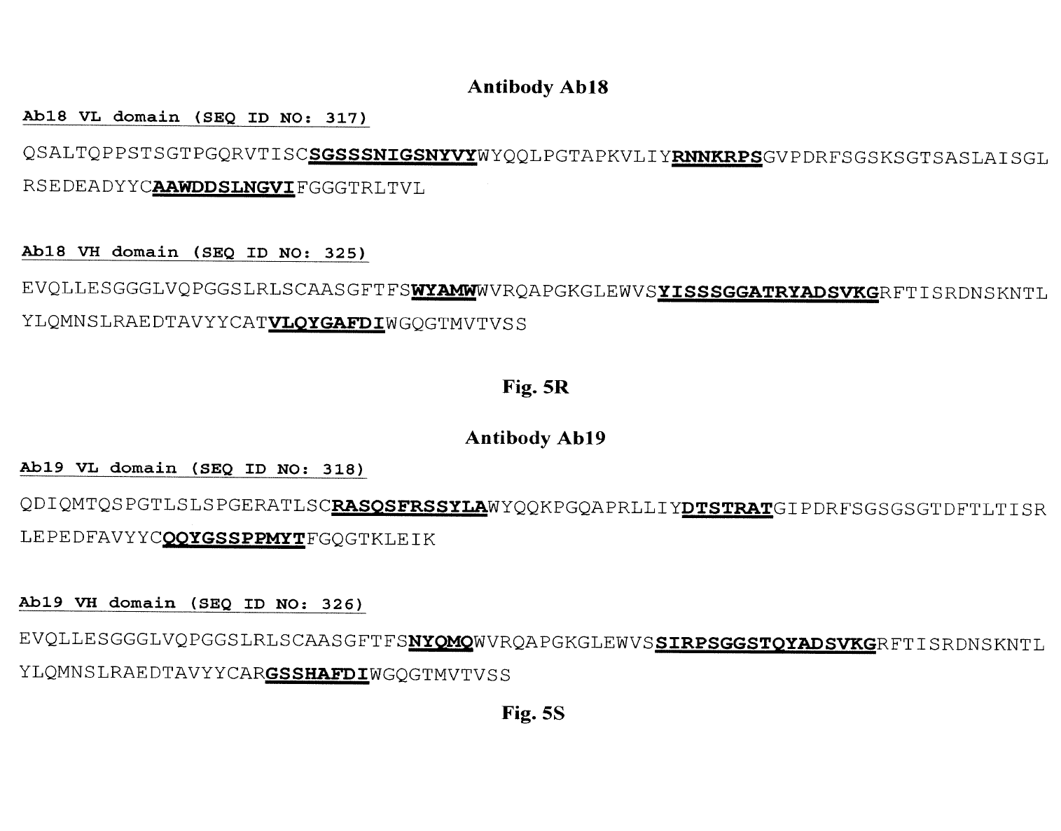

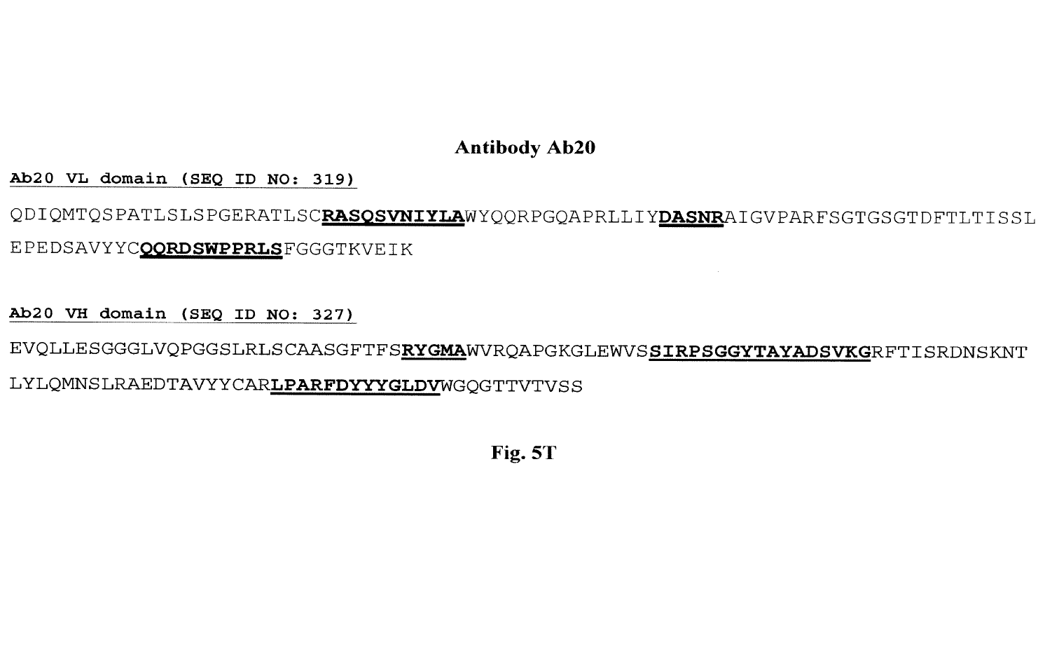

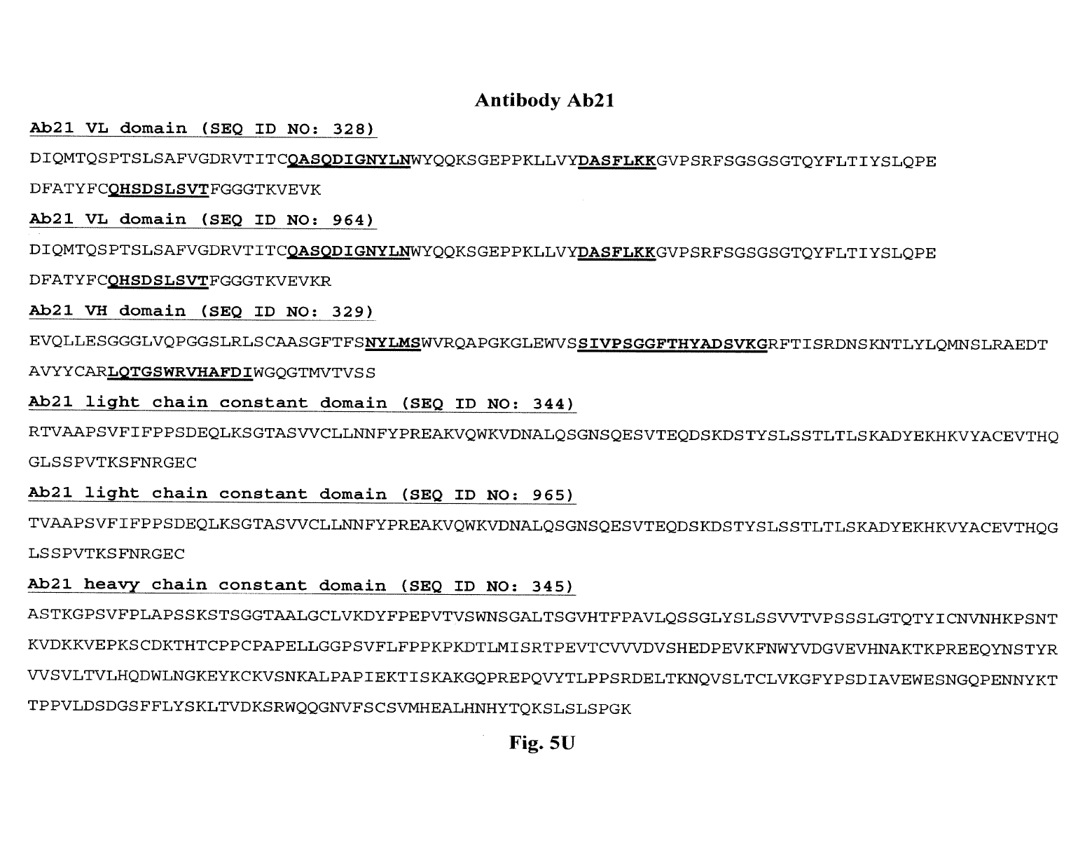

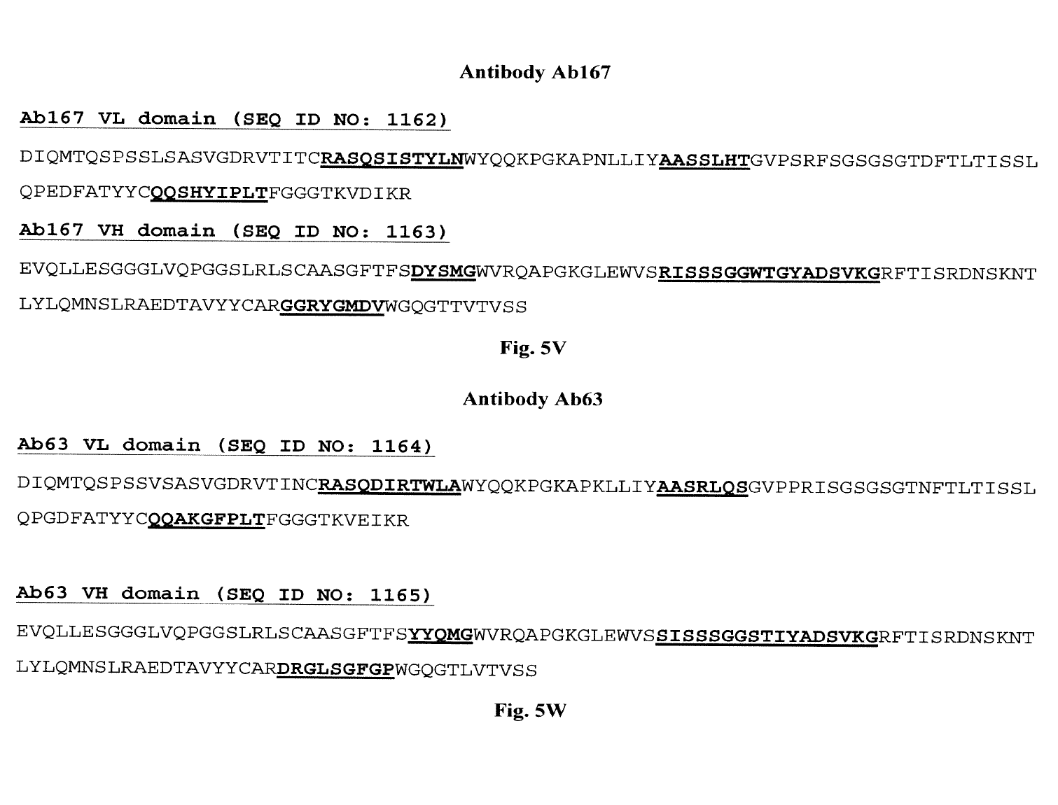

In a particular embodiment, one or two amino acid substitutions (e.g., conservative amino acid substitutions) may be present in the amino acid sequence of a CDR so long as immunospecific binding to KIT (e.g., the D4/D5 region of human KIT) is maintained. In a specific embodiment, the position of a CDR along the VH and/or VL region may vary by one to four or five amino acid position so long as immunospecific binding to KIT (e.g., the D4/D5 region of human KIT) is maintained. For example, in one embodiment, the position defining a CDR of antibody Ab1 or Ab21 can vary by shifting the N-terminal and/or C-terminal boundary of the CDR by one, two, three, four, or five amino acids, relative to the CDR position depicted in FIG. 5A for antibody Ab1 or FIG. 5U for antibody Ab21, respectively, so long as immunospecific binding to KIT (e.g., the D4/D5 region) is maintained. In another embodiment, an antibody described herein can comprise the VH CDRs (e.g., SEQ ID NOs: 86, 98, and/or 110) and VL CDRs (e.g., SEQ ID NOs: 2, 14, and/or 26) of antibody Ab1, wherein a VH CDR and/or a VL CDR contains one, two or three, amino acid substitions, such as a conservative amino acid substitution (e.g., N (asparagine) to S (serine) substitution). In one embodiment, an antibody described herein can comprise the VH CDRs (e.g., SEQ ID NOs: 86, 98, and/or 110) and VL CDRs (e.g., SEQ ID NOs: 2, 14, and/or 26) of antibody Ab1, wherein the VL CDR3 contains an amino acid substitution, such as a conservative substitution (e.g., N (asparagine) to S (serine) substitution), and wherein immunospecific binding to KIT (e.g., the D4/D5 region of human KIT) is maintained. For example, antibody Ab21 comprises the VH CDRs (e.g., SEQ ID NOs: 86, 98, and/or 110) and VL CDRs (e.g., SEQ ID NOs: 2, 14, and/or 26) of antibody Ab1, wherein the fifth amino acid of the VL CDR3 (SEQ ID NO: 110) contains the conservative substitution, N (asparagine) to S (serine) substitution, and wherein immunospecific binding to KIT (e.g., the D4/D5 region of human KIT) is maintained.

In a specific embodiment, an antibody described herein comprises one (or more) CDR(s) comprising one, two, or three amino acid additions or deletions, for example at the N-terminus and/or C-terminus, to the sequence of a CDR described herein (e.g., CDR of antibody Ab1 and/or Ab21, so long as immunospecific binding to KIT (e.g., the D4/D5 region) is maintained. In another specific embodiment, an antibody described herein comprises one (or more) CDR(s) comprising one, two, or three amino acid additions or deletions, for example at the N-terminus and/or C-terminus, to the sequence of any one of SEQ ID NOs: 2, 14, 16, 86, 98, 110, 330, 331, 332, 337, 338, and 339, so long as immunospecific binding to KIT (e.g., the D4/D5 region) is maintained.

In another specific embodiment, an antibody described herein comprises the VL CDRs (e.g., 330, 331, and/or 332) and VH CDRs (e.g., 337, 338, and/or 339) of antibody Ab21, wherein a VH CDR and/or a VL CDR contains one, two or three, amino acid substitions, such as a conservative amino acid substitution (e.g., S (serine) to N (asparagine) substitution). In another specific embodiment, an antibody described herein comprises the VL CDRs (e.g., 330, 331, and/or 332) and VH CDRs (e.g., 337, 338, and/or 339) of antibody Ab21, wherein the fifth amino acid of the VL CDR3 (SEQ ID NO: 332) contains the conservative substitution S (serine) to N (asparagine) substitution, and wherein immunospecific binding to KIT (e.g., the D4/D5 region of human KIT) is maintained.

In yet another specific embodiment, an antibody described herein comprises the VL CDRs and/or VH CDRs of an antibody comprising a VL domain (e.g., SEQ ID NO: 176, 350, or 963) and/or a VH domain (e.g., SEQ ID NO: 188) described in any one of FIGS. 5A-5W (e.g., antibody Ab1 or Ab21), wherein the position of one (or more) VL CDRs and/or VH CDRs may change by one, two, three or four amino acid positions so long as immunospecific binding to KIT (e.g., the D4/D5 region of human KIT) is maintained. In a specific embodiment, an antibody described herein comprises the VL CDRs and/or VHCDRs of antibody Ab1 comprising SEQ ID NO: 176 or 350 (VL domain) and SEQ ID NO: 188 (VH domain), wherein the position of one (or more) VL CDRs and/or VH CDRs may change by one, two, three or four amino acid positions so long as immunospecific binding to KIT (e.g., the D4/D5 region of human KIT) is maintained. In another specific embodiment, an antibody described herein comprises the VL CDRs and/or VHCDRs of antibody Ab21 comprising SEQ ID NO: 328 (VL domain) and SEQ ID NO: 329 (VH domain), wherein the position of one (or more) VL CDRs and/or VH CDRs may change by one, two, three or four amino acid positions so long as immunospecific binding to KIT (e.g., the D4/D5 region of human KIT) is maintained.

In certain embodiments, the last two, three, or four amino acids of a VH CDR2 are optional. In such an embodiment, the last one, two, three or four amino acids of such VH CDR2, may be modified by one, two, three or four amino acid substitutions (e.g., conservative substitution), deletions, and/or additions. In a specific embodiment, the last one, two, three or four amino acids of a VH CDR, such as a VH CDR described in any one of Tables 3A-3C, may be modified by one, two, three or four amino acid substitutions (e.g., conservative substitution), deletions, and/or additions. In a particular embodiment, the last one, two, three or four amino acids of the VH CDR of antibody Ab1 or Ab21, may be modified by one, two, three or four amino acid substitutions (e.g., conservative substitution), deletions, and/or additions.

In certain aspects, the CDRs of an antibody described herein is determined according to the method of Chothia and Lesk, 1987, J. Mol. Biol., 196:901-917, which will be referred to herein as the “Chothia CDRs” (see also, e.g., U.S. Pat. No. 709,226). Using the Kabat numbering system of numbering amino acid residues in the VH chain region and VL chain region, Chothia CDRs within an antibody heavy chain molecule are typically present at amino acid positions 26 to 32 (“CDR1”), amino acid positions 53 to 55 (“CDR2”), and amino acid positions 96 to 101 (“CDR3”). Using the Chothia numbering system of numbering amino acid residues in the VH chain region and VL chain region, Chothia CDRs within an antibody light chain molecule are typically present at amino acid positions 26 to 33 (CDR1), amino acid positions 50 to 52 (CDR2), and amino acid positions 91 to 96 (CDR3).

In a specific embodiment, an antibody described herein comprises CDRs as defined by the Chothia numbering system. In a particular embodiment, an antibody described herein comprises CDRs of any one of antibodies, Ab1 to Ab21, and preferably antibody Ab1 or Ab21, as defined by the Chothia numbering system. For example, in a certain embodiment, an antibody described herein comprises CDRs of antibody Ab1, wherein the antibody comprises (i) a VL CDR1 having the amino acid sequence at position 26 to 33 of SEQ ID NO:350 or 963, a VL CDR2 having the amino acid sequence at positions 50 to 52 of SEQ ID NO:350 or 963, and a VL CDR3 having the amino acid sequence at positions 91 to 96 of SEQ ID NO:350 or 963; and/or (ii) a VH CDR1 having the amino acid sequence at position 26 to 32 of SEQ ID NO:188, a VH CDR2 having the amino acid sequence at positions 53 to 55 of SEQ ID NO:188, and a VH CDR3 having the amino acid sequence at positions 96 to 101 of SEQ ID NO:188. In another certain embodiment, an antibody described herein comprises CDRs of antibody Ab21, wherein the antibody comprises (i) a VL CDR1 having the amino acid sequence at position 26 to 33 of SEQ ID NO:328 or 964, a VL CDR2 having the amino acid sequence at positions 50 to 52 of SEQ ID NO:328 or 964, and a VL CDR3 having the amino acid sequence at positions 91 to 96 of SEQ ID NO:328 or 964; and/or (ii) a VH CDR1 having the amino acid sequence at position 26 to 32 of SEQ ID NO:329, a VH CDR2 having the amino acid sequence at positions 53 to 55 of SEQ ID NO:329, and a VH CDR3 having the amino acid sequence at positions 96 to 101 of SEQ ID NO:329.

TABLE 1A
VL CDR Amino Acid Sequences

| Ab | VL CDR1 | VL CDR2 | VL CDR3 |
|---|---|---|---|
| Ab1 | QASQDIGNYLN | DASFLKK | QHSDNLSVT |
| | (SEQ ID NO: 2) | (SEQ ID NO: 14) | (SEQ ID NO: 26) |
| Ab2 | RASQSVSNYVA | DTSKRAT | QQRSYWFA |
| | (SEQ ID NO: 3) | (SEQ ID NO: 15) | (SEQ ID NO: 27) |
| Ab3 | SGSSSNIGNNYVY | RSNQRPS | AAWDDSLIGPV |
| | (SEQ ID NO: 4) | (SEQ ID NO: 16) | (SEQ ID NO: 28) |
| Ab4 | RASQSIDTYLN | AASKLED | QQSYSSPGIT |
| | (SEQ ID NO: 5) | (SEQ ID NO: 17) | (SEQ ID NO: 29) |
| Ab5 | RASQSIDTYLN | AASKLED | QQSYSSPGIT |
| | (SEQ ID NO: 6) | (SEQ ID NO: 18) | (SEQ ID NO: 30) |
| Ab6 | RASQSVGSSYLA | DASSRSS | QQRSNRPPWT |
| | (SEQ ID NO: 7) | (SEQ ID NO: 19) | (SEQ ID NO: 31) |
| Ab7 | RASQSIDTYLN | AASKLED | QQSYSSPGIT |
| | (SEQ ID NO: 8) | (SEQ ID NO: 20) | (SEQ ID NO: 32) |
| Ab8 | RASQSVSFNLA | DASTRAI | QQYNYPASYT |
| | (SEQ ID NO: 9) | (SEQ ID NO: 21) | (SEQ ID NO: 33) |
| Ab9 | RASQSVSSYLA | DASNRAT | QQRSNGGIT |
| | (SEQ ID NO: 10) | (SEQ ID NO: 22) | (SEQ ID NO: 34) |
| Ab10 | RASQSVRGNLA | GASTRAT | QQYDSWPPFT |
| | (SEQ ID NO: 11) | (SEQ ID NO: 23) | (SEQ ID NO: 35) |
| Ab11 | RASQSVSSSYLA | GASSRAT | QQYGSSRTWT |
| | (SEQ ID NO: 12) | (SEQ ID NO: 24) | (SEQ ID NO: 36) |
| Ab12 | RASQSVKSNYLA | GASTRAT | QQYGSSLSYT |
| | (SEQ ID NO: 13) | (SEQ ID NO: 25) | (SEQ ID NO: 37) |

TABLE 1B
VL CDR Amino Acid Sequences

| Ab | VL CDR1 | VL CDR2 | VL CDR3 |
|---|---|---|---|
| Ab13 | RASQSVRSNYLA | GASSRAT | QQRSNWPIT |
| | (SEQ ID NO: | (SEQ ID NO: 208) | (SEQ ID NO: 216) |
| | 200) | | |
| Ab14 | RASQGVSTYLA | GASTRAT | QQRRSWPPT |
| | (SEQ ID NO: | (SEQ ID NO: 209) | (SEQ ID NO: 217) |
| | 201) | | |
| Ab15 | RASQSVSSSYLA | GASSRAT | QQYGSSPPYT |
| | (SEQ ID NO: | (SEQ ID NO: 210) | (SEQ ID NO: 218) |
| | 202) | | |
| Ab16 | RASQSVSSSYLA | GASSRAT | QQYGSSWT |
| | (SEQ ID NO: | (SEQ ID NO: 211) | (SEQ ID NO: 219) |
| | 203) | | |
| Ab17 | RASQNIDNYLN | AASSLQS | QQSYSNSRT |
| | (SEQ ID NO: | (SEQ ID NO: 212) | (SEQ ID NO: 220) |
| | 204) | | |
| Ab18 | SGSSSNIGSNYVY | RNNKRPS | AAWDDSLNGVI |
| | (SEQ ID NO: | (SEQ ID NO: 213) | (SEQ ID NO: 221) |
| | 205) | | |
| Ab19 | RASQSFRSSYLA | DTSTRAT | QQYGSSPPMYT |
| | (SEQ ID NO: | (SEQ ID NO: 214) | (SEQ ID NO: 222) |
| | 206) | | |
| Ab20 | RASQSVNIYLA | DASNR | QQRDSWPPRLS |
| | (SEQ ID NO: | (SEQ ID NO: 215) | (SEQ ID NO: 223) |
| | 207) | | |
| Ab21 | QASQDIGNYLN | DASFLKK | QHSDSLSVT |
| | (SEQ ID NO: | (SEQ ID NO: 331) | (SEQ ID NO: 332) |
| | 330) | | |

TABLE 1C
VL CDR Amino Acid Sequences

| Ab | VL CDR1 | VL CDR2 | VL CDR3 |
|---|---|---|---|
| Ab24 | RASQSVGSNFLA | GASTRAT | QQSRSTLYT |
| | (SEQ ID NO: 351) | (SEQ ID NO: 23) | (SEQ ID NO: 859) |
| Ab25 | RASQNINSWLA | KASTLES | QQSNSFPLT |
| | (SEQ ID NO: 352) | (SEQ ID NO: 616) | (SEQ ID NO: 860) |
| Ab26 | RASQGIANYLS | PASTLQS | QQSYITLFT |
| | (SEQ ID NO: 353) | (SEQ ID NO: 617) | (SEQ ID NO: 861) |
| Ab27 | RASQTVSNNFLA | RASSRAT | QNYGYSPPYT |
| | (SEQ ID NO: 354) | (SEQ ID NO: 618) | (SEQ ID NO: 862) |
| Ab28 | RASQSISTWVA | KASTLEN | QQYKFYSSYT |
| | (SEQ ID NO: 355) | (SEQ ID NO: 619) | (SEQ ID NO: 863) |
| Ab29 | RASQSIRTNLA | GASNRAT | QQRTNWLIT |
| | (SEQ ID NO: 356) | (SEQ ID NO: 620) | (SEQ ID NO: 864) |
| Ab30 | RASQSISRWLA | KASNLEN | QQYNTYSWT |
| | (SEQ ID NO: 357) | (SEQ ID NO: 621) | (SEQ ID NO: 865) |
| Ab31 | RASQSVSSYLA | DASNRAT | QQRSNWPIT |
| | (SEQ ID NO: 10) | (SEQ ID NO: 22) | (SEQ ID NO: 216) |
| Ab32 | RASQSVSTSLA | GASTRAT | QQRSNWIT |
| | (SEQ ID NO: 358) | (SEQ ID NO: 23) | (SEQ ID NO: 866) |
| Ab33 | RGDRLRTYYSS | GRNNRPS | SSRDGSGNFL |
| | (SEQ ID NO: 359) | (SEQ ID NO: 622) | (SEQ ID NO: 867) |
| Ab34 | RASQGIYTYLV | DASTR | QQRSTWPRT |
| | (SEQ ID NO: 360) | (SEQ ID NO: 623) | (SEQ ID NO: 868) |
| Ab35 | RASRSISDWLA | KASSLES | QQYNSYSQT |
| | (SEQ ID NO: 361) | (SEQ ID NO: 624) | (SEQ ID NO: 869) |
| Ab36 | RASQSVSTNYLGTSLA | GASSRAK | QHYGRSGPWT |
| | (SEQ ID NO: 362) | (SEQ ID NO: 625) | (SEQ ID NO: 870) |
| Ab38 | RASQSIDTYLN | AASKLED | QQSYSSPGIT |
| | (SEQ ID NO: 5) | (SEQ ID NO: 17) | (SEQ ID NO: 29) |
| Ab39 | RASQSISKYLN | AASSLQS | LQSHSTPLT |
| | (SEQ ID NO: 363) | (SEQ ID NO:212) | (SEQ ID NO: 871) |
| Ab40 | RASESIGFWVA | RTSTLQS | QQYDKYPIT |
| | (SEQ ID NO: 364) | (SEQ ID NO: 626) | (SEQ ID NO: 872) |
| Ab41 | RASQSVSSYLA | DASNRAT | QQRSNWPPFT |
| | (SEQ ID NO: 10) | (SEQ ID NO:22) | (SEQ ID NO: 873) |
| Ab42 | RASQSVSKYLN | VVSNLQS | QQSYSTPQT |
| | (SEQ ID NO: 365) | (SEQ ID NO: 627) | (SEQ ID NO: 874) |
| Ab43 | RASQSVRGRLA | DASTRAT | QQYNNWPPMFT |
| | (SEQ ID NO: 366) | (SEQ ID NO: 628) | (SEQ ID NO: 875) |
| Ab44 | RASQRISNYVN | AASSLQS | QQSYSLPFT |
| | (SEQ ID NO: 367) | (SEQ ID NO:212) | (SEQ ID NO: 876) |
| Ab45 | RASQSVSSSYLA | GASSRAT | QQRSNWPSIT |
| | (SEQ ID NO: 12) | (SEQ ID NO: 24) | (SEQ ID NO: 877) |
| Ab46 | RASQSISTYLN | AASTLQN | QQSRSTLYT |
| | (SEQ ID NO: 368) | (SEQ ID NO: 629) | (SEQ ID NO: 859) |
| Ab47 | RASQRVGTNFLA | GTSTRAA | QQYEASPYT |
| | (SEQ ID NO: 369) | (SEQ ID NO: 630) | (SEQ ID NO: 878) |
| Ab48 | RASQSVSSSYLA | DASKRAT | QQRSNWPLT |
| | (SEQ ID NO: 12) | (SEQ ID NO: 631) | (SEQ ID NO: 879) |
| Ab49 | RASQGISNSLA | AASRLES | QQYYSTPFT |
| | (SEQ ID NO: 370) | (SEQ ID NO: 632) | (SEQ ID NO: 880) |
| Ab50 | RSGQNINKYLN | AASNVQG | QESFRSSRT |
| | (SEQ ID NO: 371) | (SEQ ID NO: 633) | (SEQ ID NO: 881) |
| Ab51 | QGDSLRSYYAT | GNNNRPS | KSRDSSRNHVV |
| | (SEQ ID NO: 372) | (SEQ ID NO: 634) | (SEQ ID NO: 882) |
| Ab52 | RASQGMSNWLA | DASSLQS | LQVHSFPLT |
| | (SEQ ID NO: 373) | (SEQ ID NO: 635) | (SEQ ID NO: 883) |
| Ab53 | RASQSVSSYLA | DASNRAT | QQRSNWPPIT |
| | (SEQ ID NO: 10) | (SEQ ID NO: 22) | (SEQ ID NO: 884) |
| Ab54 | RASQSIDIYLN | AASKLED | QQSYSSPGIT |
| | (SEQ ID NO: 374) | (SEQ ID NO: 17) | (SEQ ID NO: 29) |
| Ab55 | RASQGISNLLA | DASTRAT | HQRSYRGT |
| | (SEQ ID NO: 375) | (SEQ ID NO: 628) | (SEQ ID NO: 885) |
| Ab56 | RASRNVYRYLN | ASSTLQN | QQSYQTPFT |
| | (SEQ ID NO: 376) | (SEQ ID NO: 636) | (SEQ ID NO: 886) |
| Ab57 | RASQSVSSYLA | DASNRAT | QQYGSSPRT |
| | (SEQ ID NO: 10) | (SEQ ID NO: 22) | (SEQ ID NO: 887) |
| Ab58 | RASQSLGSYLA | DASNRAT | QRRSNWPPGFT |
| | (SEQ ID NO: 377) | (SEQ ID NO: 22) | (SEQ ID NO: 888) |
| Ab59 | RASQSISRYLN | AASSLQS | QQTYNTPIT |
| | (SEQ ID NO: 378) | (SEQ ID NO: 212) | (SEQ ID NO: 889) |
| Ab60 | RASQSVSSSYLA | GASNRAT | QQYGNSPPIT |
| | (SEQ ID NO: 12) | (SEQ ID NO: 620) | (SEQ ID NO: 890) |
| Ab61 | QASQDIGNYLN | DASFLKK | QHSDNLSVT |
| | (SEQ ID NO: 2) | (SEQ ID NO: 14) | (SEQ ID NO: 26) |
| Ab62 | RASQGISSALA | DASSLES | QQFNSYPLT |
| | (SEQ ID NO: 379) | (SEQ ID NO: 637) | (SEQ ID NO: 891) |
| Ab63 | RSSQSLVHSDGNTYLN | KVSNRDS | MQGTHWPPLT |
| | (SEQ ID NO: 380) | (SEQ ID NO: 638) | (SEQ ID NO: 892) |
| Ab64 | RASQSISTYLN | GASKLQD | QQSYSTPLNT |
| | (SEQ ID NO: 368) | (SEQ ID NO: 639) | (SEQ ID NO: 893) |
| Ab65 | TGSSSDVGRYNYVS | DVSERPS | CSYAGTYI |
| | (SEQ ID NO: 381) | (SEQ ID NO: 640) | (SEQ ID NO: 894) |
| Ab66 | RASQSISRYLN | AASKLED | QQSYSSPGIT |
| | (SEQ ID NO: 378) | (SEQ ID NO: 17) | (SEQ ID NO: 29) |
| Ab67 | RASQSVSSNLA | GASTRAT | QQYNNWPPWT |
| | (SEQ ID NO: 382) | (SEQ ID NO: 23) | (SEQ ID NO: 895) |
| Ab68 | RASQSVSSSYLA | GASSRAT | QQYGSSPPYT |
| | (SEQ ID NO: 12) | (SEQ ID NO: 24) | (SEQ ID NO: 218) |
| Ab69 | RASQSVSSSYLA | GASSRAT | QQYGSSWT |
| | (SEQ ID NO: 12) | (SEQ ID NO: 24) | (SEQ ID NO: 219) |
| Ab70 | TGATRDVS | EVTSRPS | SSSTSTTPRVV |
| | (SEQ ID NO: 383) | (SEQ ID NO: 641) | (SEQ ID NO: 220) |
| Ab71 | RASQSIGVSLA | KASSLES | QQSYSTLWT |
| | (SEQ ID NO: 384) | (SEQ ID NO: 624) | (SEQ ID NO: 896) |
| Ab72 | TLSSGHSSYIIA | LEGSGSYNKGS | ETWDSNTQV |
| | (SEQ ID NO: 385) | (SEQ ID NO: 642) | (SEQ ID NO: 897) |
| Ab73 | SGDRLGDKYVS | EDSKRPS | QAWDRDVV |
| | (SEQ ID NO: 386) | (SEQ ID NO: 643) | (SEQ ID NO: 898) |
| Ab74 | RASQGISNYLA | AASTLQS | QKYNSAPPLT |
| | (SEQ ID NO: 387) | (SEQ ID NO: 644) | (SEQ ID NO: 899) |
| Ab75 | RASQSVSSYLA | DASNRAT | QQRSNGGIT |
| | (SEQ ID NO: 10) | (SEQ ID NO: 22) | (SEQ ID NO: 34) |
| Ab76 | RASRNIKSYLA | KASKL | QQYDSSRT |
| | (SEQ ID NO: 388) | (SEQ ID NO: 645) | (SEQ ID NO: 900) |
| Ab77 | RASQSIGNNLG | GASTRAT | HQYDSRPRT |
| | (SEQ ID NO: 389) | (SEQ ID NO: 23) | (SEQ ID NO: 901) |
| Ab78 | QGDSLRRYYAS | GQNTRPS | DSRESSANRVI |
| | (SEQ ID NO: 390) | (SEQ ID NO: 646) | (SEQ ID NO: 902) |
| Ab79 | SGTSSDVGAYNYVS | DVNKRPS | TSYAGSNNPYV |
| | (SEQ ID NO: 391) | (SEQ ID NO: 647) | (SEQ ID NO: 903) |
| Ab80 | RASQSVSSYLA | DASNRAT | QQRSNWPLT |
| | (SEQ ID NO: 10) | (SEQ ID NO: 22) | (SEQ ID NO: 879) |
| Ab81 | RASQSISSYLN | AASSLQS | QQSYSTPPFT |
| | (SEQ ID NO: 392) | (SEQ ID NO: 212) | (SEQ ID NO: 904) |
| Ab82 | TGASGDVGGYNYVS | EGSKRPS | SSYTTSSTLV |
| | (SEQ ID NO: 393) | (SEQ ID NO: 648) | (SEQ ID NO: 905) |
| Ab83 | RASQSVSSNYLA | GASSRAT | QQYGSSRGFT |
| | (SEQ ID NO: 394) | (SEQ ID NO: 24) | (SEQ ID NO: 906) |
| Ab84 | RASQDIRTWLA | AASRLQS | QQAKGFPLT |
| | (SEQ ID NO: 395) | (SEQ ID NO: 649) | (SEQ ID NO: 907) |
| Ab85 | RASQSIAAWLA | SASTLQS | QQANSFPAT |
| | (SEQ ID NO: 396) | (SEQ ID NO: 650) | (SEQ ID NO: 962) |
| Ab86 | RASQSVSSSYLA | GASSRAT | QQRSNWPLALT |
| | (SEQ ID NO: 12) | (SEQ ID NO: 24) | (SEQ ID NO: 908) |
| Ab87 | RASQSVSSYLA | DASNRAT | QQSGRSTRT |
| | (SEQ ID NO: 10) | (SEQ ID NO:22) | (SEQ ID NO: 909) |
| Ab88 | RASQSVSSYLA | DASNRAT | QQRSNWPLT |
| | (SEQ ID NO: 10) | (SEQ ID NO:22) | (SEQ ID NO: 879) |
| Ab89 | RASQSVSNRFLA | GTSNRAT | QQYGRSPFT |
| | (SEQ ID NO: 397) | (SEQ ID NO: 651) | (SEQ ID NO: 910) |
| Ab90 | RASQSIDTYLN | AASKLED | QQSYSSPGIT |
| | (SEQ ID NO: 5) | (SEQ ID NO: 17) | (SEQ ID NO: 29) |
| Ab91 | RASQSVSSNLA | GASTRAT | QQRSNWPPSFT |
| | (SEQ ID NO: 382) | (SEQ ID NO: 23) | (SEQ ID NO: 911) |
| Ab92 | RASQSVGINLA | GASSLQG | QQSYTIPLT |
| | (SEQ ID NO: 398) | (SEQ ID NO: 652) | (SEQ ID NO: 912) |
| Ab93 | TGSTSDVGGYTYVS | DVSKRPS | CSYAGSYSYV |
| | (SEQ ID NO: 399) | (SEQ ID NO: 653) | (SEQ ID NO: 913) |
| Ab94 | RASQSVSSSYLA | GASSRAS | QQYGSSVT |
| | (SEQ ID NO: 12) | (SEQ ID NO: 654) | (SEQ ID NO: 914) |
| Ab95 | RASQGISRWLA | AATVLQP | QQTHSFSLS |
| | (SEQ ID NO: 400) | (SEQ ID NO: 655) | (SEQ ID NO: 915) |
| Ab96 | RASQTINRYLN | DASTLQR | QQSWT |
| | (SEQ ID NO: 401) | (SEQ ID NO: 656) | (SEQ ID NO: 916) |
| Ab97 | RASQSISSWLA | KASSLES | QQYNSYPWT |
| | (SEQ ID NO: 402) | (SEQ ID NO: 624) | (SEQ ID NO: 917) |
| Ab98 | RASQSISSWLA | KASSLES | QQYNSYSWT |
| | (SEQ ID NO: 402) | (SEQ ID NO: 624) | (SEQ ID NO: 918) |
| Ab99 | SGDKLGDKFVS | QDKKRPA | QAWDSSPV |
| | (SEQ ID NO: 403) | (SEQ ID NO: 657) | (SEQ ID NO: 919) |
| Ab100 | RTSQSVSSNSLA | GASSRAT | QQRYNWPPT |
| | (SEQ ID NO: 404) | (SEQ ID NO: 24) | (SEQ ID NO: 920) |
| Ab101 | RASQGISNYLA | AASTLQS | QQYKNWPQT |
| | (SEQ ID NO: 387) | (SEQ ID NO: 644) | (SEQ ID NO: 921) |
| Ab102 | RSSQSLLHSNGYTYLE | LGSNRAS | IQTLQTPYT |
| | (SEQ ID NO: 405) | (SEQ ID NO: 658) | (SEQ ID NO: 922) |
| Ab103 | RASQGIRNDLG | AASSLQR | LQHNSYPLT |
| | (SEQ ID NO: 406) | (SEQ ID NO: 659) | (SEQ ID NO: 923) |
| Ab104 | RSSQSLVYSDGSTYLN | KVSNRDS | MQGTHWPWT |
| | (SEQ ID NO: 407) | (SEQ ID NO: 638) | (SEQ ID NO: 924) |
| Ab105 | HSSHSLLYSDGKTYVN | MVSDRDS | MQGSYFPLT |
| | (SEQ ID NO: 408) | (SEQ ID NO: 660) | (SEQ ID NO: 925) |
| Ab106 | RASQSIDNHLI | GASSMQS | QQSNRSPLT |
| | (SEQ ID NO: 409) | (SEQ ID NO: 661) | (SEQ ID NO: 926) |
| Ab107 | RASESVSSSHLA | GSSNRAT | QHYGSSPGA |
| | (SEQ ID NO: 410) | (SEQ ID NO: 662) | (SEQ ID NO: 927) |
| Ab108 | TGTSSDVGRYPYVS | EVSKRPS | SSYAGSRWV |
| | (SEQ ID NO: 411) | (SEQ ID NO: 663) | (SEQ ID NO: 928) |
| Ab109 | RASQSLSSSYFA | GASNRAA | QHYSGSPWT |
| | (SEQ ID NO: 412) | (SEQ ID NO: 664) | (SEQ ID NO: 929) |
| Ab110 | RASQGISSWLA | AASSLQS | QQANNFPLT |
| | (SEQ ID NO: 413) | (SEQ ID NO:212) | (SEQ ID NO: 930) |
| Ab111 | TGTSTDVGGYNFVS | EVTNRPS | TSYSDTSPYV |
| | (SEQ ID NO: 414) | (SEQ ID NO: 665) | (SEQ ID NO: 931) |
| Ab112 | RASQRVPSKYLA | DASSRAT | QLFNRPAPWT |
| | (SEQ ID NO: 415) | (SEQ ID NO: 666) | (SEQ ID NO: 932) |
| Ab113 | SGDKLGDKYTS | QDSKRPS | QAWDSSTV |
| | (SEQ ID NO: 416) | (SEQ ID NO: 667) | (SEQ ID NO: 933) |
| Ab114 | RASQGISNYLA | ATSTLQS | QQYYRTPLT |
| | (SEQ ID NO: 387) | (SEQ ID NO: 668) | (SEQ ID NO: 934) |
| Ab115 | SGNTLSNKFVS | QDTKRPS | QTWDTGSVV |
| | (SEQ ID NO: 417) | (SEQ ID NO: 669) | (SEQ ID NO: 935) |
| Ab116 | RAGQNIGTYLN | AASSLQS | QQSYSTPGT |
| | (SEQ ID NO: 418) | (SEQ ID NO: 212) | (SEQ ID NO: 936) |
| Ab117 | RASQSVSSSYLA | GASSRAT | QQRSNWPLT |
| | (SEQ ID NO: 12) | (SEQ ID NO: 24) | (SEQ ID NO: 879) |
| Ab118 | RGDRLRSYYSS | GRNNRPS | SSRDGSGNFL |
| | (SEQ ID NO: 419) | (SEQ ID NO: 622) | (SEQ ID NO: 867 |
| Ab119 | RASQNVRNYLA | DASNRAT | QQRSNWPIT |
| | (SEQ ID NO: 420) | (SEQ ID NO: 22) | (SEQ ID NO: 216) |
| Ab120 | RASQSLSSSYLA | GASSRAT | QQYGRSAWT |
| | (SEQ ID NO: 421) | (SEQ ID NO: 24) | (SEQ ID NO: 937) |
| Ab121 | RASQSIDTYLN | AASKLED | QQSYSSPGIT |
| | (SEQ ID NO: 5) | (SEQ ID NO: 17) | (SEQ ID NO: 29) |
| Ab122 | RASQNINTYLN | TASSLQS | HQSYSTPFT |
| | (SEQ ID NO: 422) | (SEQ ID NO: 960) | (SEQ ID NO: 938) |
| Ab123 | QGDSLRKNYAS | GQNNRPS | NSRDISAKHWF |
| | (SEQ ID NO: 423) | (SEQ ID NO: 670) | (SEQ ID NO: 939) |
| Ab124 | RASQSIRSNYLA | DGSSRAN | QQYGNSPPIT |
| | (SEQ ID NO: 424) | (SEQ ID NO: 671) | (SEQ ID NO: 890) |
| Ab125 | RASQSISSNLA | DASTRAT | QQYNSWPLT |
| | (SEQ ID NO: 425) | (SEQ ID NO: 628) | (SEQ ID NO: 940) |
| Ab126 | RASQDISTWLA | AASSLQS | QQAKSFPFS |
| | (SEQ ID NO: 426) | (SEQ ID NO: 212) | (SEQ ID NO: 941) |
| Ab127 | AGTSSDVGSYNYVS | DVTNRPS | SSRTRSGTYV |
| | (SEQ ID NO: 427) | (SEQ ID NO: 672) | (SEQ ID NO: 942) |
| Ab128 | RASQSIATYLN | AASTLQN | QQSRSTLYT |
| | (SEQ ID NO: 428) | (SEQ ID NO: 629) | (SEQ ID NO: 859) |
| Ab129 | SGETLAYKYVS | EDFKRPS | QAWDRSTVV |
| | (SEQ ID NO: 429) | (SEQ ID NO: 673) | (SEQ ID NO: 943) |
| Ab130 | RASQSVSSYLA | DASNRAT | QQRSNWPRALT |
| | (SEQ ID NO: 10) | (SEQ ID NO:22) | (SEQ ID NO: 944) |
| Ab131 | SGSGSNIGTNYVY | RNNQRPS | AAWDDSLSAWV |
| | (SEQ ID NO: 430) | (SEQ ID NO: 674) | (SEQ ID NO: 945) |
| Ab132 | RASQNINIWLA | KASILHN | QQYDTYPYT |
| | (SEQ ID NO: 431) | (SEQ ID NO: 675) | (SEQ ID NO: 946) |
| Ab133 | TGGRRDIGNYNYVS | DVRKRPS | GSYTGTSNV |
| | (SEQ ID NO: 432) | (SEQ ID NO: 676) | (SEQ ID NO: 947) |
| Ab134 | RASQSVSSYLA | DASNRAT | QQRSNWPPWT |
| | (SEQ ID NO: 10) | (SEQ ID NO:22) | (SEQ ID NO: 948) |
| Ab135 | TLSSRYSTYAIA | FNTDGSQNKGD | QTWGRGIVV |
| | (SEQ ID NO: 433) | (SEQ ID NO: 677) | (SEQ ID NO: 949) |
| Ab136 | SGDKLGDKYVS | QDAKRPS | QAWDSSAVV |
| | (SEQ ID NO: 434) | (SEQ ID NO: 678) | (SEQ ID NO: 950) |
| Ab137 | RSSQSLLHSNGYNYLD | LASNRAS | MQALQTPLT |
| | (SEQ ID NO: 435) | (SEQ ID NO: 679) | (SEQ ID NO: 951) |
| Ab138 | RASQHISSWLA | KTSNLES | QHYSSYSYT |
| | (SEQ ID NO: 436) | (SEQ ID NO: 680) | (SEQ ID NO: 952) |
| Ab139 | RSSQSLLHSNGYNYLD | LGSNRAS | MQALQTPIT |
| | (SEQ ID NO: 435) | (SEQ ID NO: 658) | (SEQ ID NO: 953) |
| Ab140 | RGDRLRTYYSS | GRNNRPS | SSRDGSGNFL |
| | (SEQ ID NO: 359) | (SEQ ID NO: 622) | (SEQ ID NO: 867 |
| Ab141 | RASQSVRTYLA | DASNRAT | QQRGNWPLT |
| | (SEQ ID NO: 437) | (SEQ ID NO:22) | (SEQ ID NO: 954) |
| Ab142 | RASQSVSSTYLA | GASSRAT | QQYGRSPWT |
| | (SEQ ID NO: 438) | (SEQ ID NO: 24) | (SEQ ID NO: 955) |
| Ab143 | RASQSIDTYLN | AASKLED | QQSYSSPGIT |
| | (SEQ ID NO: 5) | (SEQ ID NO: 17) | (SEQ ID NO: 29) |
| Ab144 | TGTSSDIGGYNYVS | EVNKRPS | SSYTSSSWV |
| | (SEQ ID NO: 439) | (SEQ ID NO: 681) | (SEQ ID NO: 956) |
| Ab145 | RASQSVSSSYLA | GASSRAT | QQYGSSLFT |
| | (SEQ ID NO: 12) | (SEQ ID NO: 24) | (SEQ ID NO: 957) |
| Ab146 | RASQSVGSNFLA | GASTRAT | QQYGSSPRT |
| | (SEQ ID NO: 351) | (SEQ ID NO: 23) | (SEQ ID NO: 887) |
| Ab147 | RASQSVSSYLA | DASNRAT | QQRSNWPPKLT |
| | (SEQ ID NO: 10) | (SEQ ID NO: 22) | (SEQ ID NO: 958) |
| Ab148 | RASQSVSNYLA | GASSRAT | QQYGSSPQT |
| | (SEQ ID NO: 440) | (SEQ ID NO: 24) | (SEQ ID NO: 959) |
| Ab149 | RASQAISNYLA | SASTLQS | QKYNTGLFT |
| | (SEQ ID NO: 441) | (SEQ ID NO: 650) | (SEQ ID NO: 969) |
| Ab150 | RASQSVNIYLA | DASNR | QQRDSWPPRLS |
| | (SEQ ID NO: 207) | (SEQ ID NO: 215) | (SEQ ID NO: 223) |
| Ab151 | RASQSISSYLN | AASSLQS | QQSYSTPLT |
| | (SEQ ID NO: 392) | (SEQ ID NO: 212) | (SEQ ID NO: 970) |
| Ab152 | RASQSVSSSYLA | GASSRAT | QQYGSSRWT |
| | (SEQ ID NO: 12) | (SEQ ID NO: 24) | (SEQ ID NO: 971) |
| Ab153 | RASQSVTRNYLA | GTSTRAT | QQHASG |
| | (SEQ ID NO: 442) | (SEQ ID NO: 682) | (SEQ ID NO: 972) |
| Ab154 | RASQGVSRWLA | AASSLQS | QQSYSTPPT |
| | (SEQ ID NO: 443) | (SEQ ID NO:212) | (SEQ ID NO: 973) |
| Ab155 | RASQGISSWLA | AASSLQS | QQANSFPLT |
| | (SEQ ID NO: 413) | (SEQ ID NO:212) | (SEQ ID NO: 974) |
| Ab156 | RASQSISSYLN | AASSLQS | QQSYSTRDLLT |
| | (SEQ ID NO: 392) | (SEQ ID NO:212) | (SEQ ID NO: 975) |
| Ab157 | RASQSISSWLA | GASSRAT | QQYGSSLLT |
| | (SEQ ID NO: 402) | (SEQ ID NO: 24) | (SEQ ID NO: 976) |
| Ab158 | TGTSSDIGGYNFVS | QVSDRPS | SSYTTSNTLV |
| | (SEQ ID NO: 444) | (SEQ ID NO: 683) | (SEQ ID NO: 977) |
| Ab159 | RSSQRLLHSNGYNYLD | LGSNRAS | MQALQTPIT |
| | (SEQ ID NO: 445) | (SEQ ID NO: 658) | (SEQ ID NO: 953) |
| Ab160 | SGDKLGDKYVS | QDYKRPS | QAWDSSTYVV |
| | (SEQ ID NO: 434) | (SEQ ID NO: 684) | (SEQ ID NO: 978) |
| Ab161 | SGDKLGDKYAS | QDRKRPS | QAWDSNTVV |
| | (SEQ ID NO: 446) | (SEQ ID NO: 685) | (SEQ ID NO: 979) |
| Ab162 | RASQGIRNDLA | AASGLQS | LQHSTYPLT |
| | (SEQ ID NO: 447) | (SEQ ID NO: 686) | (SEQ ID NO: 980) |
| Ab163 | SGDELGEKYAS | QDNKRPS | QAWDTTTFYV |
| | (SEQ ID NO: 448) | (SEQ ID NO: 687) | (SEQ ID NO: 981) |
| Ab164 | KSSQSVLYSSSNKNYLA | WASTRES | QQYYSTPLT |
| | (SEQ ID NO: 449) | (SEQ ID NO: 688) | (SEQ ID NO: 982) |
| Ab165 | SGGSSNIGSNFVY | RNYQRPS | AAWDDNVGGV |
| | (SEQ ID NO: 450) | (SEQ ID NO: 689) | (SEQ ID NO: 983) |
| Ab166 | RASQSVSSHLA | NAFNRAT | QHRVNGLFI |
| | (SEQ ID NO: 451) | (SEQ ID NO: 690) | (SEQ ID NO: 984) |
| Ab167 | RASQSISTYLN | AASSLHT | QQSHYIPLT |
| | (SEQ ID NO: 368) | (SEQ ID NO: 691) | (SEQ ID NO: 985) |
| Ab168 | SGSSSNIGGYYVY | RNNQRPS | AAWDDSLSGVV |
| | (SEQ ID NO: 452) | (SEQ ID NO: 674) | (SEQ ID NO: 986) |
| Ab169 | RSSQNINNFVN | AASRLKG | QQSFINPQT |
| | (SEQ ID NO: 453) | (SEQ ID NO: 692) | (SEQ ID NO: 987) |
| Ab170 | RASQSIDTYLN | AASKLED | QQSYSSPGIT |
| | (SEQ ID NO: 5) | (SEQ ID NO: 17) | (SEQ ID NO: 29) |
| Ab171 | RASQSVSSSYLA | GASSRAT | QQYGRSRYT |
| | (SEQ ID NO: 12) | (SEQ ID NO: 24) | (SEQ ID NO: 988) |
| Ab172 | RASQSISTSLN | AASKLED | QQSHSASWT |
| | (SEQ ID NO: 454) | (SEQ ID NO: 17) | (SEQ ID NO: 989) |
| Ab173 | RASQGISNSLA | AASRLES | QQYYSTPRT |
| | (SEQ ID NO: 370) | (SEQ ID NO: 632) | (SEQ ID NO: 990) |
| Ab174 | RASQGISSSLA | GASRLES | QQYNSYPVT |
| | (SEQ ID NO: 455) | (SEQ ID NO: 693) | (SEQ ID NO: 991) |
| Ab175 | RASQSVNSVYLA | NASNRAT | QQANTFPLT |
| | (SEQ ID NO: 456) | (SEQ ID NO: 694) | (SEQ ID NO: 992) |
| Ab176 | RASQSVSNYLN | ATSNLQS | QQTYNIVFT |
| | (SEQ ID NO: 457) | (SEQ ID NO: 695) | (SEQ ID NO: 993) |
| Ab177 | SGGNSNIGSHYVY | RDNQRPS | AAWDDSLGGPV |
| | (SEQ ID NO: 458) | (SEQ ID NO: 696) | (SEQ ID NO: 994) |
| Ab178 | RASQSVSYWLA | RASRLES | QQYNNESGGT |
| | (SEQ ID NO: 459) | (SEQ ID NO: 697) | (SEQ ID NO: 995) |
| Ab179 | RASQSVSSYLA | DASNRAT | QQRSNWPPIFT |
| | (SEQ ID NO: 10) | (SEQ ID NO:22) | (SEQ ID NO: 996) |
| Ab180 | RASQSVSSSYLA | GASNRAT | QQYGNSPPIT |
| | (SEQ ID NO: 12) | (SEQ ID NO: 620) | (SEQ ID NO: 890) |
| Ab181 | RSSQSLLHSNGYNYLD | LGSNRAS | MQALQTPLS |
| | (SEQ ID NO: 435) | (SEQ ID NO: 658) | (SEQ ID NO: 997) |
| Ab182 | RASQSVRSSYLA | GASTRAT | QQYGTSRWT |
| | (SEQ ID NO: 460) | (SEQ ID NO: 23) | (SEQ ID NO: 998) |
| Ab183 | RASQSVSSYLA | DASNRAT | QQRSNGGIT |
| | (SEQ ID NO: 10) | (SEQ ID NO:22) | (SEQ ID NO: 34) |
| Ab184 | RASQTIGDNLA | GASMRAT | QQRSSWPLT |
| | (SEQ ID NO: 461) | (SEQ ID NO: 698) | (SEQ ID NO: 999) |
| Ab185 | RASQSVSSYLA | DASNRAT | QQRSNWPLT |
| | (SEQ ID NO: 10) | (SEQ ID NO:22) | (SEQ ID NO: 879) |
| Ab186 | QASQDISNYLN | DASNLET | QQANSFPRT |
| | (SEQ ID NO: 462) | (SEQ ID NO: 699) | (SEQ ID NO: 1000) |
| Ab187 | SGDRLQTKFIS | QDYKRPS | QAWDNSRFI |
| | (SEQ ID NO: 463) | (SEQ ID NO: 684) | (SEQ ID NO: 1001) |
| Ab188 | RASQGISSWLA | AASSLQS | QQANRFPLT |
| | (SEQ ID NO: 413) | (SEQ ID NO:212) | (SEQ ID NO: 1002) |
| Ab189 | SGSSSNIGSNYVY | RNNQRPS | AAWDDSLSGWV |
| | (SEQ ID NO: 205) | (SEQ ID NO: 674) | (SEQ ID NO: 1003) |
| Ab190 | RASQSVGSYLA | DTSNRAT | QQRSNWRIT |
| | (SEQ ID NO: 464) | (SEQ ID NO: 700) | (SEQ ID NO: 1004) |
| Ab191 | QGDSLRSYYAS | GKNNRPS | NSRDSSGNHLGVV |
| | (SEQ ID NO: 465) | (SEQ ID NO: 701) | (SEQ ID NO: 1005) |
| Ab192 | RASQTINRWLA | ATSTLEN | QQYNVSPYT |
| | (SEQ ID NO: 466) | (SEQ ID NO: 702) | (SEQ ID NO: 1006) |

TABLE 2A
VL FR Amino Acid Sequences

| Ab | VL FR1 | VL FR2 | VL FR3 | VL FR4 |
|---|---|---|---|---|
| Ab1 | QDIQMTQSPTSLSAFVGDRVTITC | WYQQKSGEPPKLLVY | GVPSRFSGSGSGTQYFLTIYSLQPEDFATYFC | FGGGTKVEVK |
| | (SEQ ID NO: 38) | (SEQ ID NO: 50) | (SEQ ID NO: 62) | (SEQ ID NO: 74) |
| Ab2 | QDIQMTQSPATLSLSPGERATLYC | WYQQRPGQAPRLLMY | GIPARFSGSGSGTDFTLTISSLEPEDFAVYYC | FGGGTKVEIK |
| | (SEQ ID NO: 39) | (SEQ ID NO: 51) | (SEQ ID NO: 63) | (SEQ ID NO: 75) |
| Ab3 | QSALTQPPSASGTPGQRVTISC | WYQQLPGTAPKLLIY | GVPDRFSGSKSGASASLAISGLRSEDEADYYC | FGGGTKLTVL |
| | (SEQ ID NO: 40) | (SEQ ID NO: 52) | (SEQ ID NO: 64) | (SEQ ID NO: 76) |
| Ab4 | QDIQMTQSPSSLSASVGDRVAITC | WYQQKPGKAPKLLIY | GVPSRFSGSGTGTDFTLTIRSLQPEDFASYFC | FGPGTKVEIK |
| | (SEQ ID NO: 41) | (SEQ ID NO: 53) | (SEQ ID NO: 65) | (SEQ ID NO: 77) |
| Ab5 | QDIQMTQSPSSLSASVGDRVAITC | WYQQKPGKAPKLLIY | GVPSRFSGSGTGTDFTLTIRSLQPEDFASYFC | FGPGTKVEIK |
| | (SEQ ID NO: 42) | (SEQ ID NO: 54) | (SEQ ID NO: 66) | (SEQ ID NO: 78) |
| Ab6 | QDIQMTQSPATLSLYPGERATLSC | WYQQKPGQAPRLLIY | GIPDRFSGSGSGTDFTLTITRLEPEDFAVYYC | FGQGTKVEIK |
| | (SEQ ID NO: 43) | (SEQ ID NO: 55) | (SEQ ID NO: 67) | (SEQ ID NO: 79) |
| Ab7 | QDIQMTQSPSSLSASVGDRVAITC | WYQQKPGKAPKLLIY | GVPSRFSGSGTGTDFTLTIRSLQPEDFASYFC | FGPGTKVEIK |
| | (SEQ ID NO: 44) | (SEQ ID NO: 56) | (SEQ ID NO: 68) | (SEQ ID NO: 80) |
| Ab8 | QDIQMTQSPVTLSLSPGDRATLSC | WYQHKPGQAPRLLMF | GTPDRFSGSGSGTEFTLTISSLQSEDSAVYYC | FGQGTKLEIS |
| | (SEQ ID NO: 45) | (SEQ ID NO: 57) | (SEQ ID NO: 69) | (SEQ ID NO: 81) |
| Ab9 | QDIQMTQSPATLSLSPGERATLSC | WYQQKPGQAPRLLIY | GIPARFSGSGSGTDFTLTISSLEPEDFAVYYC | FGQGTRLEIK |
| | (SEQ ID NO: 46) | (SEQ ID NO: 58) | (SEQ ID NO: 70) | (SEQ ID NO: 82) |
| Ab10 | QDIQMTQSPDSLSVSPGERATLSC | WYQQKPGQAPRLLIH | DIPTRFSGSGSGTDFTLTISSMQSEDFAVYYC | FGPGTKVDIK |
| | (SEQ ID NO: 47) | (SEQ ID NO: 59) | (SEQ ID NO: 71) | (SEQ ID NO: 83) |
| Ab11 | QDIQMTQSPGTLSLSPGERATLSC | WYQQKPGQAPRLLIY | GIPDRFSGSGSGTDFTLTISRLEPEDFAVYYC | FGQGTKVEIK |
| | (SEQ ID NO: 48) | (SEQ ID NO: 60) | (SEQ ID NO: 72) | (SEQ ID NO: 84) |
| Ab12 | QDIQMTQSPGTLSLSPGERATLYC | WYQQKPGQAPRLLIY | DIPDRFSGSGSGTDFTLTISRLEPEDFAVYYC | FGQGTKLEIK |
| | (SEQ ID NO: 49) | (SEQ ID NO: 61) | (SEQ ID NO: 73) | (SEQ ID NO: 85) |

TABLE 2B
VL FR Amino Acid Sequences

| Ab | VL FR1 | VL FR2 | VL FR3 | VL FR4 |
|---|---|---|---|---|
| Ab13 | QDIQMTQSPGTLSLSPGERATLSC | WYQQKPGQAPRLLIY | GIPDRFSGSGSGTDFTLTISRLEPEDFAVYYC | FGQGTRLEIK |
| | (SEQ ID NO: 224) | (SEQ ID NO: 232) | (SEQ ID NO: 240) | (SEQ ID NO: 248) |
| Ab14 | QDIQMTQSPATLSVSPGERATLSC | WYQQKPGQAPRLLIY | GIPARFSGSGSGTDFTLTINSLELEDFAVYYC | FGQGTKVEIK |
| | (SEQ ID NO: 225) | (SEQ ID NO: 233) | (SEQ ID NO: 241) | (SEQ ID NO: 249) |
| Ab15 | QDIQMTQSPGTLSLSPGERATLSC | WYQQKPGQAPRLLIY | GIPDRFSGSGSGTDFTLTISRLEPEDFAVYYC | FGQGTKLEIK |
| | (SEQ ID NO: 226) | (SEQ ID NO: 234) | (SEQ ID NO: 242) | (SEQ ID NO: 250) |
| Ab16 | QDIQMTQSPGTLSLSPGERATLSC | WYQQKPGQAPRLLIY | GIPDRFSGSGSGTDFTLTISRLEPEDFAVYYC | FGQGTKVEIK |
| | (SEQ ID NO: 227) | (SEQ ID NO: 235) | (SEQ ID NO: 243) | (SEQ ID NO: 251) |
| Ab17 | QDIQMTQSPSSLSASVGDRVTVTC | WYQQRPGKAPELLIY | GVPSRFSGSGSGTDFTLTISSLQPEDFATYYC | FGQGTKVEIK |
| | (SEQ ID NO: 228) | (SEQ ID NO: 236) | (SEQ ID NO: 244) | (SEQ ID NO: 252) |
| Ab18 | QSALTQPPSTSGTPGQRVTISC | WYQQLPGTAPKVLIY | GVPDRFSGSKSGTSASLAISGLRSEDEADYYC | FGGGTRLTVL |
| | (SEQ ID NO: 229) | (SEQ ID NO: 237) | (SEQ ID NO: 245) | (SEQ ID NO: 253) |
| Ab19 | QDIQMTQSPGTLSLSPGERATLSC | WYQQKPGQAPRLLIY | GIPDRFSGSGSGTDFTLTISRLEPEDFAVYYC | FGQGTKLEIK |
| | (SEQ ID NO: 230) | (SEQ ID NO: 238) | (SEQ ID NO: 246) | (SEQ ID NO: 254) |
| Ab20 | QDIQMTQSPATLSLSPGERATLSC | WYQQRPGQAPRLLIY | AIGVPARFSGTGSGTDFTLTISSLEPEDSAVYYC | FGGGTKVEIK |
| | (SEQ ID NO: 231) | (SEQ ID NO: 239) | (SEQ ID NO: 247) | (SEQ ID NO: 255) |
| Ab21 | DIQMTQSPTSLSAFVGDRVTITC | WYQQKSGEPPKLLVY | GVPSRFSGSGSGTQYFLTIYSLQPEDFATYFC | FGGGTKVEVK |
| | (SEQ ID NO: 333) | (SEQ ID NO: 334) | (SEQ ID NO: 335) | (SEQ ID NO: 336) |

TABLE 3A
VH CDR Amino Acid Sequences

| Ab | VH CDR1 | VH CDR2 | VH CDR3 |
|---|---|---|---|
| Ab1 | NYLMS | SIVPSGGFTHYADSVKG | LQTGSWRVHAFDI |
| | (SEQ ID NO: 86) | (SEQ ID NO: 98) | (SEQ ID NO: 110) |
| Ab2 | NYKMG | WIYPSGGGTFQADSVKG | VRVGSTGNFDY |
| | (SEQ ID NO: 87) | (SEQ ID NO: 99) | (SEQ ID NO: 111) |
| Ab3 | PYNMM | RISPSGGDTHYADSVKG | GSRSSWSTPYY |
| | (SEQ ID NO: 88) | (SEQ ID NO: 100) | (SEQ ID NO: 112) |
| Ab4 | WYWMY | GIYPSGGFTSYADSVKG | SYYYGSGSYYNSASFDY |
| | (SEQ ID NO: 89) | (SEQ ID NO: 101) | (SEQ ID NO: 113) |
| Ab5 | SYPMA | VISSSGGYTPYADSVKG | RYGGGMDV |
| | (SEQ ID NO: 90) | (SEQ ID NO: 102) | (SEQ ID NO: 114) |
| Ab6 | YYSMG | VIRPSGGHTHYADSVKG | LATNAFDI |
| | (SEQ ID NO: 91) | (SEQ ID NO: 103) | (SEQ ID NO: 115) |
| Ab7 | RYPMN | GIRSSGGATSYADSVKG | SFGRAFDY |
| | (SEQ ID NO: 92) | (SEQ ID NO: 104) | (SEQ ID NO: 116) |
| Ab8 | NYKMQ | YISSSGGYTSYADSVKG | VAHQYYDFWSGYYGAFDI |
| | (SEQ ID NO: 93) | (SEQ ID NO: 105) | (SEQ ID NO: 117) |
| Ab9 | NYLMQ | GIVPSGGSTVYADSVKG | RGGY |
| | (SEQ ID NO: 94) | (SEQ ID NO: 106) | (SEQ ID NO: 118) |
| Ab10 | IYSMM | WIVSSGGQTYYADSVKG | GRGSTAFDI |
| | (SEQ ID NO: 95) | (SEQ ID NO: 107) | (SEQ ID NO: 119) |
| Ab11 | NYRMM | GIYPSGGGTFYADSVKG | KKMATFAAFDI |
| | (SEQ ID NO: 96) | (SEQ ID NO: 108) | (SEQ ID NO: 120) |
| Ab12 | FYKMH | GIWPSGGYTEYADSVKG | SSGYHIDY |
| | (SEQ ID NO: 97) | (SEQ ID NO: 109) | (SEQ ID NO: 121) |

TABLE 3B
VH CDR Amino Acid Sequences

| Ab | VH CDR1 | VH CDR2 | VH CDR3 |
|---|---|---|---|
| Ab13 | NYTMH | VIYPSGGMTKYADSVKG | GSTTFDY |
| | (SEQ ID NO: 256) | (SEQ ID NO: 264) | (SEQ ID NO: 272) |
| Ab14 | RYAMW | YIYSSGGQTGYADSVKG | GGVTFDL |
| | (SEQ ID NO: 257) | (SEQ ID NO: 265) | (SEQ ID NO: 273) |
| Ab15 | NYTMM | GIRPSGGGTMYADSVKG | VVNHKFDN |
| | (SEQ ID NO: 258) | (SEQ ID NO: 266) | (SEQ ID NO: 274) |
| Ab16 | QYVMF | SIYPSGGHTLYADSVKG | HARGYNRGYFDS |
| | (SEQ ID NO: 259) | (SEQ ID NO: 267) | (SEQ ID NO: 275) |
| Ab17 | NYTMI | RIWPSGGGTDYADSVKG | RVGASRYFDL |
| | (SEQ ID NO: 260) | (SEQ ID NO: 268) | (SEQ ID NO: 276) |
| Ab18 | WYAMW | YISSSGGATRYADSVKG | VLQYGAFDI |
| | (SEQ ID NO: 261) | (SEQ ID NO: 269) | (SEQ ID NO: 277) |
| Ab19 | NYQMQ | SIRPSGGSTQYADSVKG | GSSHAFDI |
| | (SEQ ID NO: 262) | (SEQ ID NO: 270) | (SEQ ID NO: 278) |
| Ab20 | RYGMA | SIRPSGGYTAYADSVKG | LPARFDYYYGLDV |
| | (SEQ ID NO: 263) | (SEQ ID NO: 271) | (SEQ ID NO: 279) |
| Ab21 | NYLMS | SIVPSGGFTHYADSVKG | LQTGSWRVHAFDI |
| | (SEQ ID NO: 337) | (SEQ ID NO: 338) | (SEQ ID NO: 339) |

TABLE 3C
VH CDR Amino Acid Sequences

| Ab | VH CDR1 | VH CDR2 | VH CDR3 |
|---|---|---|---|
| Ab24 | SYMMV | YIRSSGGYTSYADSVKG | APGYYYYGMDV |
| | (SEQ ID NO: 467) | (SEQ ID NO: 961) | (SEQ ID NO: 1007) |
| Ab25 | AYQMH | SIVPSGGSTNYADSVKG | SRTLDY* |
| | (SEQ ID NO: 468) | (SEQ ID NO: 703) | (SEQ ID NO: 1008) |
| Ab26 | YYGMG | RISSSGGGTRYADSVKG | ELGPLLGGRGY |
| | (SEQ ID NO: 469) | (SEQ ID NO: 704) | (SEQ ID NO: 1009) |
| Ab27 | NYSMH | YIYPSGGGTIYADSVKG | RAAGYGGAFDI |
| | (SEQ ID NO: 470) | (SEQ ID NO: 705) | (SEQ ID NO: 1010) |
| Ab28 | NYRMR | GIYPSGGDTQYADSVKG | SGNLDY |
| | (SEQ ID NO: 471) | (SEQ ID NO: 706) | (SEQ ID NO: 1011) |
| Ab29 | NYRMG | SIVPSGGSTQYADSVKG | GPQYFDY |
| | (SEQ ID NO: 472) | (SEQ ID NO: 707) | (SEQ ID NO: 1012) |
| Ab30 | RYSMR | SISPSGGYTEYADSVKG | LPPGD |
| | (SEQ ID NO: 473) | (SEQ ID NO: 708) | (SEQ ID NO: 1013) |
| Ab31 | RYWMG | SISSSGGYTNYADSVKG | QGGGSFTRDY |
| | (SEQ ID NO: 474) | (SEQ ID NO: 709) | (SEQ ID NO: 1014) |
| Ab32 | NYRMN | YIWPSGGGTYADSVKG | GTVGAFDI |
| | (SEQ ID NO: 475) | (SEQ ID NO: 710) | (SEQ ID NO: 1015) |
| Ab33 | WYPMI | WISPSGGFTNYADSVKG | DRSGTHDY |
| | (SEQ ID NO: 476) | (SEQ ID NO: 711) | (SEQ ID NO: 1016) |
| Ab34 | DYFMF | VISPSGGWTEYADSVKG | DWRVMVNPFDY |
| | (SEQ ID NO: 477) | (SEQ ID NO: 712) | (SEQ ID NO: 1017) |
| Ab35 | VYPML | SISSSGGDTAYADSVKG | PAYSSSPGDY |
| | (SEQ ID NO: 478) | (SEQ ID NO: 713) | (SEQ ID NO: 1018) |
| Ab36 | NYKMI | RIYPSGGNTGYADSVKG | EAIGRAFDI |
| | (SEQ ID NO: 479) | (SEQ ID NO: 714) | (SEQ ID NO: 1019) |
| Ab38 | KYPMA | VIRSSGGFTTYADSVKG | GYGHGLDY |
| | (SEQ ID NO: 480) | (SEQ ID NO: 715) | (SEQ ID NO: 1020) |
| Ab39 | FYAMA | RISSSGGNTQYADSVKG | DPMGVVGADY |
| | (SEQ ID NO: 481) | (SEQ ID NO: 716) | (SEQ ID NO: 1021) |
| Ab40 | HYSMG | SISPSGGATVYADSVKG | DMTTVPKLGVYYYGMDV |
| | (SEQ ID NO: 482) | (SEQ ID NO: 717) | (SEQ ID NO: 1022) |
| Ab41 | LYPMV | VISSSGGHTRYADSVKG | IVGGAFDI |
| | (SEQ ID NO: 483) | (SEQ ID NO: 718) | (SEQ ID NO: 1023) |
| Ab42 | NYQMT | SIWSSGGKTEYADSVKG | GRYSYGLDV |
| | (SEQ ID NO: 484) | (SEQ ID NO: 719) | (SEQ ID NO: 1024) |
| Ab43 | NYTMN | SIVPSGGNTTYADSVKG | DGAGERRGAFDI |
| | (SEQ ID NO: 485) | (SEQ ID NO: 720) | (SEQ ID NO: 1025) |
| Ab44 | NYHMM | VISPSGGFTAYADSVKG | VTGYPYLFDY |
| | (SEQ ID NO: 486) | (SEQ ID NO: 721) | (SEQ ID NO: 1026) |
| Ab45 | LYTMT | SISPSGGYTFYADSVKG | VGYSKNY |
| | (SEQ ID NO: 487) | (SEQ ID NO: 722) | (SEQ ID NO: 1027) |
| Ab46 | PYSMV | SIRPSGGNTRYADSVKG | GIAVAGWAFDI |
| | (SEQ ID NO: 488) | (SEQ ID NO: 723) | (SEQ ID NO: 1028) |
| Ab47 | RYGMK | SISPSGGLTIYADSVKG | PGP |
| | (SEQ ID NO: 489) | (SEQ ID NO: 724) | (SEQ ID NO: 1029) |
| Ab48 | QYKMY | VISSSGGMTEYADSVKG | ERGSSGGVNYYYYGMDV |
| | (SEQ ID NO: 490) | (SEQ ID NO: 725) | (SEQ ID NO: 1030) |
| Ab49 | RYSMT | SIRSSGGITQYADSVKG | RYSGSFDY |
| | (SEQ ID NO: 491) | (SEQ ID NO: 726) | (SEQ ID NO: 1031) |
| Ab50 | NYSMK | SIYPSGGGTMYADSVKG | DGGAHAFDI |
| | (SEQ ID NO: 492) | (SEQ ID NO: 727) | (SEQ ID NO: 1032) |
| Ab51 | HYEMM | RIVSSGGKTHYADSVKG | QDY |
| | (SEQ ID NO: 493) | (SEQ ID NO: 728) | (SEQ ID NO: 1033) |
| Ab52 | KYMMF | GIYSSGGWTGYADSVKG | ASVGAFDY |
| | (SEQ ID NO: 494) | (SEQ ID NO: 729) | (SEQ ID NO: 1034) |
| Ab53 | NYAMA | RIWPSGGGTLYADSVKG | GNVHFDY |
| | (SEQ ID NO: 495) | (SEQ ID NO: 730) | (SEQ ID NO: 1035) |
| Ab54 | KYGMN | GIYPSGGETAYADSVKG | RPRVGSTGSVLDS |
| | (SEQ ID NO: 496) | (SEQ ID NO: 731) | (SEQ ID NO: 1036) |
| Ab55 | YYTMF | RIVPSGGSTNYADSVKG | HSGGAFDI |
| | (SEQ ID NO: 497) | (SEQ ID NO: 732) | (SEQ ID NO: 1037) |
| Ab56 | HYHMS | RIYSSGGSTMYADSVKG | DRGSYGMDV |
| | (SEQ ID NO: 498) | (SEQ ID NO: 733) | (SEQ ID NO: 1038) |
| Ab57 | KYEMQ | GISPSGGITHYADSVKG | GLVTYYYDSSGYYRWGYYGMDV |
| | (SEQ ID NO: 499) | (SEQ ID NO: 734) | (SEQ ID NO: 1039) |
| Ab58 | NYSMW | RIYPSGGNTFYADSVKG | LTGSYSDY |
| | (SEQ ID NO: 500) | (SEQ ID NO: 735) | (SEQ ID NO: 1040) |
| Ab59 | AYVMS | SIGPSGGLTIYADSVKG | GIAVARY |
| | (SEQ ID NO: 501) | (SEQ ID NO: 736) | (SEQ ID NO: 1041) |
| Ab60 | NYSMN | GIWPSGGNTFYADSVKG | KTIGRARAFDI |
| | (SEQ ID NO: 502) | (SEQ ID NO: 737) | (SEQ ID NO: 1042) |
| Ab61 | NYLMS | SIVPSGGFTHYADSVKG | GLMGFDY |
| | (SEQ ID NO: 86) | (SEQ ID NO: 98) | (SEQ ID NO: 1043) |
| Ab62 | TYWMM | SIVPSGGFTSYADSVKG | GYSGSSRGGLRY |
| | (SEQ ID NO: 503) | (SEQ ID NO: 738) | (SEQ ID NO: 1044) |
| Ab63 | RYMMF | VISPSGGWTRYADSVKG | FRPEDW |
| | (SEQ ID NO: 504) | (SEQ ID NO: 739) | (SEQ ID NO: 1045) |
| Ab64 | GMN | YISSSGGATIYADSVKG | GYGRGAFDI |
| | (SEQ ID NO: 505) | (SEQ ID NO: 740) | (SEQ ID NO: 1046) |
| Ab65 | KYSMT | RISSSGGYTPYADSVKG | RGDGNSYLGY |
| | (SEQ ID NO: 506) | (SEQ ID NO: 741) | (SEQ ID NO: 1047) |
| Ab66 | KYHMG | WIYPSGGNTTYADSVKG | GYSRLFDY |
| | (SEQ ID NO: 507) | (SEQ ID NO: 742) | (SEQ ID NO: 1048) |
| Ab67 | NYRMM | GIYPSGGGTFYADSVKG | KKMATFAAFDI |
| | (SEQ ID NO: 96) | (SEQ ID NO: 108) | (SEQ ID NO: 120) |
| Ab68 | NYTMM | GIRPSGGGTMYADSVKG | VVNHKFDN |
| | (SEQ ID NO: 258) | (SEQ ID NO: 266) | (SEQ ID NO: 274) |
| Ab69 | QYVMF | SIYPSGGHTLYADSVKG | HARGYNRGYFDS |
| | (SEQ ID NO: 259) | (SEQ ID NO: 267) | (SEQ ID NO: 275) |
| Ab70 | WYKMG | SIYSSGGSTRYADSVKG | ALSSGWQNNWFDS |
| | (SEQ ID NO: 508) | (SEQ ID NO: 743) | (SEQ ID NO: 1049) |
| Ab71 | KYKMV | VISPSGGITHYADSVKG | GPTVVNHFQH |
| | (SEQ ID NO: 509) | (SEQ ID NO: 744) | (SEQ ID NO: 1050) |
| Ab72 | VYAMG | SISPSGGVTKYADSVKG | HLIGRDGYNYYYYGMDV |
| | (SEQ ID NO: 510) | (SEQ ID NO: 745) | (SEQ ID NO: 1051) |
| Ab73 | PYPMF | SISPSGGATHYADSVKG | IPSYWRDYGMDV |
| | (SEQ ID NO: 511) | (SEQ ID NO: 746) | (SEQ ID NO: 1052) |
| Ab74 | SYTMQ | GIYPSGGGTMYADSVKG | HKAFDI |
| | (SEQ ID NO: 512) | (SEQ ID NO: 747) | (SEQ ID NO: 1053) |
| Ab75 | NYSMS | YIYPSGGETMYADSVKG | ARGLDV |
| | (SEQ ID NO: 513) | (SEQ ID NO: 748) | (SEQ ID NO: 1054) |
| Ab76 | KYDMY | YISSSGGSTWYADSVKG | DKHRYYGMDV |
| | (SEQ ID NO: 514) | (SEQ ID NO: 749) | (SEQ ID NO: 1055) |
| Ab77 | KYVMH | RIGPSGGKTHYADSVKG | QGGGGRAFDI |
| | (SEQ ID NO: 515) | (SEQ ID NO: 750) | (SEQ ID NO: 1056) |
| Ab78 | NYTMH | RIYPSGGGTAYADSVKG | GGAAVNTSWFDP |
| | (SEQ ID NO: 256) | (SEQ ID NO: 751) | (SEQ ID NO: 1057) |
| Ab79 | YYKMA | VIYPSGGYTKYADSVKG | GPVGWTPADY |
| | (SEQ ID NO: 516) | (SEQ ID NO: 752) | (SEQ ID NO: 1058) |
| Ab80 | LYSMN | RISSSGGETQYADSVKG | GYRAFQN |
| | (SEQ ID NO: 517) | (SEQ ID NO: 753) | (SEQ ID NO: 1059) |
| Ab81 | DYFMQ | GIGPSGGPTQYADSVKG | SKWFGVSRGFDY |
| | (SEQ ID NO: 518) | (SEQ ID NO: 754) | (SEQ ID NO: 1060) |
| Ab82 | PYMML | WIVPSGGDTAYADSVKG | GSQQLVLVNWFDP |
| | (SEQ ID NO: 519) | (SEQ ID NO: 755) | (SEQ ID NO: 1061) |
| Ab83 | HYHMV | VISPSGGKTRYADSVKG | APRISSRYFDL |
| | (SEQ ID NO: 520) | (SEQ ID NO: 756) | (SEQ ID NO: 1062) |
| Ab84 | YYQMG | SISSSGGSTIYADSVKG | DRGLSGFGP |
| | (SEQ ID NO: 521) | (SEQ ID NO: 757) | (SEQ ID NO: 1063) |
| Ab85 | YYIMA | SIGSSGGMTAYADSVKG | VGLGFAVDI |
| | (SEQ ID NO: 522) | (SEQ ID NO: 758) | (SEQ ID NO: 1064) |
| Ab86 | YYKMF | GIYPSGGATEYADSVKG | DIGYGVLDP |
| | (SEQ ID NO: 523) | (SEQ ID NO: 759) | (SEQ ID NO: 1065) |
| Ab87 | NYTMN | RIWPSGGSNFYADSVKG | GNGGFDS |
| | (SEQ ID NO: 485) | (SEQ ID NO: 760) | (SEQ ID NO: 1066) |
| Ab88 | KYVMY | GIVPSGGLTQYADSVKG | ARRMRSLWEAFDV |
| | (SEQ ID NO: 524) | (SEQ ID NO: 761) | (SEQ ID NO: 1067) |
| Ab89 | WYTMT | SIWPSGGGTDYADSVKG | SPRGWGTPSFFDY |
| | (SEQ ID NO: 525) | (SEQ ID NO: 762) | (SEQ ID NO: 1068) |
| Ab90 | KYLMM | YISSSGGHTYYADSVKG | KYAGYFDY |
| | (SEQ ID NO: 526) | (SEQ ID NO: 763) | (SEQ ID NO: 1069) |
| Ab91 | YYRMW | RIGPSGGTTNYADSVKG | ETGGGFDY |
| | (SEQ ID NO: 527) | (SEQ ID NO: 764) | (SEQ ID NO: 1070) |
| Ab92 | SYQMS | YIWPSGGATSYADSVKG | VAGGVAGISIDY |
| | (SEQ ID NO: 528) | (SEQ ID NO: 765) | (SEQ ID NO: 1071) |
| Ab93 | PYPMQ | SISSSGGFTSYADSVKG | LGPGY |
| | (SEQ ID NO: 529) | (SEQ ID NO: 766) | (SEQ ID NO: 1072) |
| Ab94 | KYKMI | RISSSGGRTKYADSVKG | MGRSSRGTFDI |
| | (SEQ ID NO: 530) | (SEQ ID NO: 767) | (SEQ ID NO: 1073) |
| Ab95 | LYTMM | YIGSSGGNTNYADSVKG | TAVGRAFDI |
| | (SEQ ID NO: 531) | (SEQ ID NO: 768) | (SEQ ID NO: 1074) |
| Ab96 | QYNMS | SIYPSGGVTGYADSVKG | KTSAYDY |
| | (SEQ ID NO: 532) | (SEQ ID NO: 769) | (SEQ ID NO: 1075) |
| Ab97 | PYPMK | SIWSSGGPTQYADSVKG | EGLIYDILTGYVRPKGMDV |
| | (SEQ ID NO: 533) | (SEQ ID NO: 770) | (SEQ ID NO: 1076) |
| Ab98 | RYSMR | SISPSGGYTEYADSVKG | LPPGD |
| | (SEQ ID NO: 473) | (SEQ ID NO: 708) | (SEQ ID NO: 1013) |
| Ab99 | HYGMG | YISSSGGQTKYADSVKG | GNGMDV |
| | (SEQ ID NO: 534) | (SEQ ID NO: 771) | (SEQ ID NO: 1077) |
| Ab100 | KYSMQ | GIGSSGGLTSYADSVKG | DPNIVLIWGGQAFDI |
| | (SEQ ID NO: 535) | (SEQ ID NO: 772) | (SEQ ID NO: 1078) |
| Ab101 | NYSMH | YIYPSGGGTIYADSVKG | RAAGYGGAFDI |
| | (SEQ ID NO: 470) | (SEQ ID NO: 705) | (SEQ ID NO: 1010) |
| Ab102 | GYMMH | GIGSSGGWTSYADSVKG | GNRYSYGHYFDY |
| | (SEQ ID NO: 536) | (SEQ ID NO: 773) | (SEQ ID NO: 1079) |
| Ab103 | AYFMH | SISSSGGSTLYADSVKG | VDVRRFYGDYRQDY |
| | (SEQ ID NO: 537) | (SEQ ID NO: 774) | (SEQ ID NO: 1080) |
| Ab104 | KYTMG | YIGSSGGKTTYADSVKG | LGFCSGGDCHPDV |
| | (SEQ ID NO: 538) | (SEQ ID NO: 775) | (SEQ ID NO: 1081) |
| Ab105 | AYPMH | SIWPSGGFTIYADSVKG | DAAGGYGTFDI |
| | (SEQ ID NO: 539) | (SEQ ID NO: 776) | (SEQ ID NO: 1082) |
| Ab106 | VYAMG | GIGPSGGTTHYADSVKG | GRLERRSYYYYGMDV |
| | (SEQ ID NO: 510) | (SEQ ID NO: 777) | (SEQ ID NO: 1083) |
| Ab107 | HYKMS | GIYPSGGPTGYADSVKG | DVAGRWRGFDY |
| | (SEQ ID NO: 540) | (SEQ ID NO: 778) | (SEQ ID NO: 1084) |
| Ab108 | NYHMM | SISSGGKTYYADSVKG | GAAYSSGYSFVDY |
| | (SEQ ID NO: 486) | (SEQ ID NO: 779) | (SEQ ID NO: 1085) |
| Ab109 | TYGMQ | YISSSGGWTQYANSVKG | VKPIAAI |
| | (SEQ ID NO: 541) | (SEQ ID NO: 780) | (SEQ ID NO: 1086) |
| Ab110 | NYSMH | RIYPSGGITPYADSVKG | RLGGDYFDL |
| | (SEQ ID NO: 470) | (SEQ ID NO: 781) | (SEQ ID NO: 1087) |
| Ab111 | PYWMI | RISPSGGDTKYADSVKG | AFRYYDFWSGPTGDAFDI |
| | (SEQ ID NO: 542) | (SEQ ID NO: 782) | (SEQ ID NO: 1088) |
| Ab112 | YYNMV | SIYPSGGLTQYADSVKG | ASMGWSHPFDY |
| | (SEQ ID NO: 543) | (SEQ ID NO: 783) | (SEQ ID NO: 1089) |
| Ab113 | DYKMH | GIWPSGGATSYADSVKG | DPYSGTSYFDF |
| | (SEQ ID NO: 544) | (SEQ ID NO: 784) | (SEQ ID NO: 1090) |
| Ab114 | TYWMH | SIYSSGNTVYADSVKG | ESGVFGSAFDL |
| | (SEQ ID NO: 545) | (SEQ ID NO: 785) | (SEQ ID NO: 1091) |
| Ab115 | KYRMM | YISSSGGVTEYADSVKG | DLDY |
| | (SEQ ID NO: 546) | (SEQ ID NO: 786) | (SEQ ID NO: 1092) |
| Ab116 | FYNMW | RISSSGGFTYYADSVKG | PSSGWYRYGMDV |
| | (SEQ ID NO: 547) | (SEQ ID NO: 787) | (SEQ ID NO: 1093) |
| Ab117 | NYPMG | YIVPSGGSTAYADPVKG | IVGGGLDL |
| | (SEQ ID NO: 548) | (SEQ ID NO: 788) | (SEQ ID NO: 1094) |
| Ab118 | NYAMH | YIVPSGGSTHYADSVKG | ISSSSADY |
| | (SEQ ID NO: 549) | (SEQ ID NO: 789) | (SEQ ID NO: 1095) |
| Ab119 | HYPMH | SIVSSGGFTQYADSVKG | GGRDLDY |
| | (SEQ ID NO: 550) | (SEQ ID NO: 790) | (SEQ ID NO: 1096) |
| Ab120 | NYKMQ | VISPSGGYTAYADSVKG | TAAAGSRKAFDI |
| | (SEQ ID NO: 93) | (SEQ ID NO: 791) | (SEQ ID NO: 1097) |
| Ab121 | KYPMH | SIVSSGGTTKYADSVKG | GYGYSIDY |
| | (SEQ ID NO: 551) | (SEQ ID NO: 792) | (SEQ ID NO: 1098) |
| Ab122 | KYRMF | YISPSGGYTAYADSVKG | RSGSYGGSFDY |
| | (SEQ ID NO: 552) | (SEQ ID NO: 793) | (SEQ ID NO: 1099) |
| Ab123 | NYRMM | GIYPSGGGTFYADSVKG | KKMATFAAFDI |
| | (SEQ ID NO: 96) | (SEQ ID NO: 108) | (SEQ ID NO: 120) |
| Ab124 | WYNMQ | SIWPSGGATNYADSVKG | SPDYYGSGYYDY |
| | (SEQ ID NO: 553) | (SEQ ID NO: 794) | (SEQ ID NO: 1100) |
| Ab125 | NYRMQ | WIYPSGGGTWYADSVKG | MVRGVVHY |
| | (SEQ ID NO: 554) | (SEQ ID NO: 795) | (SEQ ID NO: 1101) |
| Ab126 | WYEMW | YIGSSGGDTWYADSVKG | VAARRYYFDY |
| | (SEQ ID NO: 555) | (SEQ ID NO: 796) | (SEQ ID NO: 1102) |
| Ab127 | YYNMM | SISPSGGHTTYADSVKG | GGSGGVSDY |
| | (SEQ ID NO: 556) | (SEQ ID NO: 797) | (SEQ ID NO: 1103) |
| Ab128 | MYRMV | SIVSSGGFTKYADSVKG | DRIGMDV |
| | (SEQ ID NO: 557) | (SEQ ID NO: 798) | (SEQ ID NO: 1104) |
| Ab129 | KYNMW | GIVPSGGYTLYADSVKG | LRSSWERWGSVDY |
| | (SEQ ID NO: 558) | (SEQ ID NO: 799) | (SEQ ID NO: 1105) |
| Ab130 | MYGMA | YISSSGGTTIYADSVKG | RIGGAFDI |
| | (SEQ ID NO: 559) | (SEQ ID NO: 800) | (SEQ ID NO: 1106) |
| Ab131 | SYGMW | SIGPSGGYTRYADSVKG | SMKGGHLDY |
| | (SEQ ID NO: 560) | (SEQ ID NO: 801) | (SEQ ID NO: 1107) |
| Ab132 | EYDMV | YISPSGGKTSYADSVKG | SGIFWYFDL |
| | (SEQ ID NO: 561) | (SEQ ID NO: 802) | (SEQ ID NO: 1108) |
| Ab133 | NYEMH | GISPSGGWTQYADSVKG | AVSSSEERYYYYGMDV |
| | (SEQ ID NO: 562) | (SEQ ID NO: 803) | (SEQ ID NO: 1109) |
| Ab134 | YYTMA | SIRPSGGSTTYADSVKG | RQVGRRAFDI |
| | (SEQ ID NO: 563) | (SEQ ID NO: 804) | (SEQ ID NO: 1110) |
| Ab135 | AYRMQ | WIYPSGGATLYADSVKG | VYAGAFDI |
| | (SEQ ID NO: 564) | (SEQ ID NO: 805) | (SEQ ID NO: 1111) |
| Ab136 | KYHMM | GIVSSGGSTAYADSVKG | HGFDI |
| | (SEQ ID NO: 565) | (SEQ ID NO: 806) | (SEQ ID NO: 1112) |
| Ab137 | KYSMR | SISSSGGTTWYADSVKG | PAGDYYGSGSN |
| | (SEQ ID NO: 566) | (SEQ ID NO: 807) | (SEQ ID NO: 1113) |
| Ab138 | WYNMH | SIWSSGGGTQYADSVKG | AYGGNFDY |
| | (SEQ ID NO: 567) | (SEQ ID NO: 808) | (SEQ ID NO: 1114) |
| Ab139 | WYPMM | RIGSSGGRTDYADSVKG | VSSYPGYAFDI |
| | (SEQ ID NO: 568) | (SEQ ID NO: 809) | (SEQ ID NO: 1115) |
| Ab140 | SYHMQ | RISPSGGSTWYADSVKG | GGGRDWPYYFDY |
| | (SEQ ID NO: 569) | (SEQ ID NO: 810) | (SEQ ID NO: 1116) |
| Ab141 | KYGMQ | YIGSSGGLTRYADSVKG | DRPSSNGDYFDY |
| | (SEQ ID NO: 570) | (SEQ ID NO: 811) | (SEQ ID NO: 1117) |
| Ab142 | HYRMA | GIYPSGGMTSYADSVKG | SITIFGLRKNYYYYGMDV |
| | (SEQ ID NO: 571) | (SEQ ID NO: 812) | (SEQ ID NO: 1118) |
| Ab143 | KYSML | SIGSSGGVTKYADSVKG | LYGGNPDY |
| | (SEQ ID NO: 572) | (SEQ ID NO: 813) | (SEQ ID NO: 1119) |
| Ab144 | KYIMT | SISPSGGYTAYADSVKG | HGDYYDSSGYYTPSGYGMDV |
| | (SEQ ID NO: 573) | (SEQ ID NO: 814) | (SEQ ID NO: 1120) |
| Ab145 | IYRMQ | SIVPSGGSTGYADSVKG | GLGGYFQH |
| | (SEQ ID NO: 574) | (SEQ ID NO: 815) | (SEQ ID NO: 1121) |
| Ab146 | SYMMV | YIRSSGGYTSYADSVKG | APGYYYYGMDV |
| | (SEQ ID NO: 467) | (SEQ ID NO: 961) | (SEQ ID NO: 1007) |
| Ab147 | IYKMS | SIWPSGGNTTYADSVKG | ESSGSLDY |
| | (SEQ ID NO: 575) | (SEQ ID NO: 816) | (SEQ ID NO: 1122) |
| Ab148 | AYVMT | SIWSSGGSTGYADSVKG | YGSGKVAFDI |
| | (SEQ ID NO: 576) | (SEQ ID NO: 817) | (SEQ ID NO: 1123) |
| Ab149 | AYSMH | RIVPSGGLTNYADSVKG | GGGSFDY |
| | (SEQ ID NO: 577) | (SEQ ID NO: 818) | (SEQ ID NO: 1124) |
| Ab150 | RYGMA | SIRPSGGYTAYADSVKG | LPARFDYYYGLDV |
| | (SEQ ID NO: 263) | (SEQ ID NO: 271) | (SEQ ID NO: 279) |
| Ab151 | VYGMG | YIYPSGGLTRYADSVKG | DFQSSGWYVYGMDV |
| | (SEQ ID NO: 578) | (SEQ ID NO: 819) | (SEQ ID NO: 1125) |
| Ab152 | NYKMV | SIYPSGGGTRYADSVKG | GSSNMLHY |
| | (SEQ ID NO: 579) | (SEQ ID NO: 820) | (SEQ ID NO: 1126) |
| Ab153 | PYHMT | SIGPSGGNTLYADSVKG | AHSSGSYYYYYGMDV |
| | (SEQ ID NO: 580) | (SEQ ID NO: 821) | (SEQ ID NO: 1127) |
| Ab154 | QYHMT | SIYSSGGLTNYADSVKG | LNYYYGLDV |
| | (SEQ ID NO: 581) | (SEQ ID NO: 822) | (SEQ ID NO: 1128) |
| Ab155 | SYSMW | VISPSGGFTRYADSVKG | SMVRGRRGFDY |
| | (SEQ ID NO: 582) | (SEQ ID NO: 823) | (SEQ ID NO: 1129) |
| Ab156 | IYGMH | SIVPSGGFTPYADSVKG | TPYYGSGYAFDI |
| | (SEQ ID NO: 583) | (SEQ ID NO: 824) | (SEQ ID NO: 1130) |
| Ab157 | KYPMG | SIVSSGGSTHYADSVKG | VYARQLDF |
| | (SEQ ID NO: 584) | (SEQ ID NO: 825) | (SEQ ID NO: 1131) |
| Ab158 | LYGMH | SIWSSGGYTTYADSVKG | SYGSGTFDI |
| | (SEQ ID NO: 585) | (SEQ ID NO: 826) | (SEQ ID NO: 1132) |
| Ab159 | KYVMI | YISSSGGNTRYADSVKG | GAPFNY |
| | (SEQ ID NO: 586) | (SEQ ID NO: 827) | (SEQ ID NO: 1133) |
| Ab160 | KYMMG | YIGPSGGMTGYADSVKG | GLVAATRYFQH |
| | (SEQ ID NO: 587) | (SEQ ID NO: 828) | (SEQ ID NO: 1134) |
| Ab161 | KYYMH | SISSSGGATFYADSVKG | SRGLEY |
| | (SEQ ID NO: 588) | (SEQ ID NO: 829) | (SEQ ID NO: 1135) |
| Ab162 | QYMMS | SISPSGGHTYYADSVKG | LPGNY |
| | (SEQ ID NO: 589) | (SEQ ID NO: 830) | (SEQ ID NO: 1136) |
| Ab163 | NYGMI | YIYPSGGGTMYADSVKG | VTPGWERAFDI |
| | (SEQ ID NO: 590) | (SEQ ID NO: 831) | (SEQ ID NO: 1137) |
| Ab164 | HYPMY | VISPSGGYTLYADSVKG | GRGWFDL |
| | (SEQ ID NO: 591) | (SEQ ID NO: 832) | (SEQ ID NO: 1138) |
| Ab165 | RYKMV | GIRPSGGLTTYADSVKG | DGGSGWYLQH |
| | (SEQ ID NO: 592) | (SEQ ID NO: 833) | (SEQ ID NO: 1139) |
| Ab166 | NYSMG | SIYPSGGSTAYADSVKG | MVRGVVDY |
| | (SEQ ID NO: 593) | (SEQ ID NO: 834) | (SEQ ID NO: 1140) |
| Ab167 | DYSMG | RISSSGGWTGYADSVKG | GGRYGMDV |
| | (SEQ ID NO: 594) | (SEQ ID NO: 835) | (SEQ ID NO: 1141) |
| Ab168 | SYQMI | YIGSSGGTTSYADSVKG | GKRGVSGSRDFMVYY |
| | (SEQ ID NO: 595) | (SEQ ID NO: 836) | (SEQ ID NO: 1142) |
| Ab169 | VYNMR | SISPSGGRTYYADSVKG | DSVRYYDSSGYYYTDYYGMDV |
| | (SEQ ID NO: 596) | (SEQ ID NO: 837) | (SEQ ID NO: 1143) |
| Ab170 | RYKMG | SIRSSGGKTLYADSVKG | GYGHGLDY |
| | (SEQ ID NO: 597) | (SEQ ID NO: 838) | (SEQ ID NO: 1020) |
| Ab171 | NYSMG | SIYPSGGSTAYADSVKG | MVRGVVDY |
| | (SEQ ID NO: 593) | (SEQ ID NO: 834) | (SEQ ID NO: 1140) |
| Ab172 | QYWMG | SIRSSGGYTLYADSVKG | RYGYGFDY |
| | (SEQ ID NO: 598) | (SEQ ID NO: 839) | (SEQ ID NO: 1144) |
| Ab173 | KYPMI | GIYSSGGMTHYADSVKG | VYGGAFDI |
| | (SEQ ID NO: 599) | (SEQ ID NO: 840) | (SEQ ID NO: 1145) |
| Ab174 | WYPMG | WIGSSGGVTRYADSVKG | VYGGNFDY |
| | (SEQ ID NO: 600) | (SEQ ID NO: 841) | (SEQ ID NO: 1146) |
| Ab175 | RYPMA | YIYPSGGHTDYADSVKG | HGLGSWYSFDY |
| | (SEQ ID NO: 601) | (SEQ ID NO: 842) | (SEQ ID NO: 1147) |
| Ab176 | NYGMF | GIRSSGGQTWYADSVKG | WHGGKSAFDY |
| | (SEQ ID NO: 602) | (SEQ ID NO: 843) | (SEQ ID NO: 1148) |
| Ab177 | VYWMH | SIRPSGGFTFYADSVKG | GPVAGGHFDY |
| | (SEQ ID NO: 603) | (SEQ ID NO: 844) | (SEQ ID NO: 1149) |
| Ab178 | KYKMA | YIGSSGGYTQYADSVKG | AWGNAFDI |
| | (SEQ ID NO: 604) | (SEQ ID NO: 845) | (SEQ ID NO: 1150) |
| Ab179 | NYTMF | GIVPSGGSTIYADSVKG | VTTNAFDI |
| | (SEQ ID NO: 605) | (SEQ ID NO: 846) | (SEQ ID NO: 1151) |
| Ab180 | NYSMN | RIYPSGGNTFYADSVKG | KTIGRARAFDI |
| | (SEQ ID NO: 502) | (SEQ ID NO: 735) | (SEQ ID NO: 1042) |
| Ab181 | KYWMN | VISPSGGSTKYADSVKG | GPAFDI |
| | (SEQ ID NO: 606) | (SEQ ID NO: 847) | (SEQ ID NO: 1152) |
| Ab182 | FYNMH | GIVPSGGMTSYADSVKG | GTTGAFDI |
| | (SEQ ID NO: 607) | (SEQ ID NO: 848) | (SEQ ID NO: 1153) |
| Ab183 | YYKMV | GIYPSGGLTQYADSVKG | GSSGSFDY |
| | (SEQ ID NO: 608) | (SEQ ID NO: 849) | (SEQ ID NO: 1154) |
| Ab184 | NYRMF | SIVPSGGSTVYADSVKG | HYDSSGRGAFDI |
| | (SEQ ID NO: 609) | (SEQ ID NO: 850) | (SEQ ID NO: 1155) |
| Ab185 | KYVMY | GIVPSGGATMYADSVKG | ARRMRSLWEAFDV |
| | (SEQ ID NO: 524) | (SEQ ID NO: 851) | (SEQ ID NO: 1067) |
| Ab186 | KYPMM | SIWSSGGQTYYADSVKG | GFGDS |
| | (SEQ ID NO: 610) | (SEQ ID NO: 852) | (SEQ ID NO: 1156) |
| Ab187 | KYGMN | SISSSGGQTEYADSVKG | NFDN |
| | (SEQ ID NO: 496) | (SEQ ID NO: 853) | (SEQ ID NO: 1157) |
| Ab188 | MYAMH | SIRPSGGATNYADSVKG | GGWELPH |
| | (SEQ ID NO: 611) | (SEQ ID NO: 854) | (SEQ ID NO: 1158) |
| Ab189 | VYTMY | YIGSSGGTTIYADSVKG | SMKGGHLDY |
| | (SEQ ID NO: 612) | (SEQ ID NO: 855) | (SEQ ID NO: 1107) |
| Ab190 | NYTMV | YIYPSGGGTSYADSVKG | FSLGNFDF |
| | (SEQ ID NO: 613) | (SEQ ID NO: 856) | (SEQ ID NO: 1159) |
| Ab191 | DYAMA | WISPSGGWTNYADSVKG | VVGGLAPFDY |
| | (SEQ ID NO: 614) | (SEQ ID NO: 857) | (SEQ ID NO: 1160) |
| Ab192 | HYSMI | VIGPSGGFTRYADSVKG | AGRGAGLATV |
| | (SEQ ID NO: 615) | (SEQ ID NO: 858) | (SEQ ID NO: 1161) |

TABLE 4A
VH FR Amino Acid Sequences

| Ab | VH FR1 | VH FR2 | VH FR3 | VH FR4 |
|---|---|---|---|---|
| Ab1 | EVQLLESGGGLVQPGG | WVRQAPGKGLEWVS | RFTISRDNSKNTLYLQMNSLRAEDTAVYYCAR | WGQGTMVTVSS |
| | SLRLSCAASGFTFS | (SEQ ID NO: 134) | (SEQ ID NO: 146) | (SEQ ID NO: 158) |
| | (SEQ ID NO: 122) | | | |
| Ab2 | EVQLLESGGGLVQPGG | WVRQAPGKGLEWVS | RFTISRDNSKNTLYLQMNSLRAEDTAVYYCAR | WGQGTLVTVSS |
| | SLRLSCAASGFTFS | (SEQ ID NO: 135) | (SEQ ID NO: 147) | (SEQ ID NO: 159) |
| | (SEQ ID NO: 123) | | | |
| Ab3 | EVQLLESGGGLVQPGG | WVRQAPGKGLEWVS | RFTISRDNSKNTLYLQMNSLRAEDTAVYYCAR | WGQGILVTVSS |
| | SLRLSCAASGFTFS | (SEQ ID NO: 136) | (SEQ ID NO: 148) | (SEQ ID NO: 160) |
| | (SEQ ID NO: 124) | | | |
| Ab4 | EVQLLESGGGLVQPGG | WVRQAPGKGLEWVS | RFTISRDNSKNTLYLQMNSLRAEDTAVYYCAR | WGQGTLVTVSS |
| | SLRLSCAASGFTFS | (SEQ ID NO: 137) | (SEQ ID NO: 149) | (SEQ ID NO: 161) |
| | (SEQ ID NO: 125) | | | |
| Ab5 | EVQLLESGGGLVQPGG | WVRQAPGKGLEWVS | RFTISRDNSKNTLYLQMNSLRAEDTAMYYCAR | WGQGTLVTVSS |
| | SLRLSCAASGFTFS | (SEQ ID NO: 138) | (SEQ ID NO: 150) | (SEQ ID NO: 162) |
| | (SEQ ID NO: 126) | | | |
| Ab6 | EVQLLESGGGLVQPGG | WVRQAPGKGLEWVS | RFTISRDNSKNTLYLQMNSLRAEDTAVYYCAR | WGQGTMVTVSS |
| | SLRLSCAASGFTFS | (SEQ ID NO: 139) | (SEQ ID NO: 151) | (SEQ ID NO: 163) |
| | (SEQ ID NO: 127) | | | |
| Ab7 | EVQLLESGGGLVQPGG | WVRQAPGKGLEWVS | RFTISRDNSKNTLYLQMNSLRAEDTAVYYCTR | WGQGTLVTVSS |
| | SLRLSCAASGFTFS | (SEQ ID NO: 140) | (SEQ ID NO: 152) | (SEQ ID NO: 164) |
| | (SEQ ID NO: 128) | | | |
| Ab8 | EVQLLESGGGLVQPGG | WVRQAPGKGLEWVS | RFTISRDNSKNTLYLQMNSLRAEDTAVYYCAR | WGQGTMVTVSS |
| | SLRLSCAASGFTFS | (SEQ ID NO: 141) | (SEQ ID NO: 153) | (SEQ ID NO: 165) |
| | (SEQ ID NO: 129) | | | |
| Ab9 | EVQLLESGGGLVQPGG | WVRQAPGKGLEWVS | RFTISRDNSKNTLYLQMNSLRAEDTAVYYCAT | WGQGTLVTVSS |
| | SLRLSCAASGFTFS | (SEQ ID NO: 142) | (SEQ ID NO: 154) | (SEQ ID NO: 166) |
| | (SEQ ID NO: 130) | | | |
| Ab10 | EVQLLESGGGLVQPGG | WVRQAPGKGLEWVS | RFTISRDNSKNTLYLQMNSLRAEDTAVYYCAR | WGQGTMVTVSS |
| | SLRLSCAASGFTFS | (SEQ ID NO: 143) | (SEQ ID NO: 155) | (SEQ ID NO: 167) |
| | (SEQ ID NO: 131) | | | |
| Ab11 | EVQLLESGGGLVQPGG | WVRQAPGKGLEWVS | RFTISRDNSKNTLYLQMNSLRAEDTAVYYCAR | WGQGTMVTVSS |
| | SLRLSCAASGFTFS | (SEQ ID NO: 144) | (SEQ ID NO: 156) | (SEQ ID NO: 168) |
| | (SEQ ID NO: 132) | | | |
| Ab12 | EVQLLESGGGLVQPGG | WVRQAPGKGLEWVS | RFTISRDNSKNTLYLQMNSLRAEDTAVYYCAR | WGQGTLVTVSS |
| | SLRLSCAASGFTFS | (SEQ ID NO: 145) | (SEQ ID NO: 157) | (SEQ ID NO: 169) |
| | (SEQ ID NO: 133) | | | |

TABLE 4B
VH FR Amino Acid Sequences

| Ab | VH FR1 | VH FR2 | VH FR3 | VH FR4 |
|---|---|---|---|---|
| Ab13 | EVQLLESGGGLVQPGG | WVRQAPGKGLEWVS | RFTISRDNSKNTLYLQMNSLRAEDTAVYYCAR | WGQGTLVTVSS |
| | SLRLSCAASGFTFS | (SEQ ID NO: 288) | (SEQ ID NO: 296) | (SEQ ID NO: 304) |
| | (SEQ ID NO: 280) | | | |
| Ab14 | EVQLLESGGGLVQPGG | WVRQAPGKGLEWVS | RFTISRDNSKNTLYLQMNSLRAEDTAVYYCAR | WGRGTLVTVSS |
| | SLRLSCAASGFTFS | (SEQ ID NO: 289) | (SEQ ID NO: 297) | (SEQ ID NO: 305) |
| | (SEQ ID NO: 281) | | | |
| Ab15 | EVQLLESGGGLVQPGG | WVRQAPGKGLEWVS | RFTISRDNSKNTLYLQMNSLRAEDTAVYYCAS | WGQGTLVTVSS |
| | SLRLSCAASGFTFS | (SEQ ID NO: 290) | (SEQ ID NO: 298) | (SEQ ID NO: 306) |
| | (SEQ ID NO: 282) | | | |
| Ab16 | EVQLLESGGGLVQPGG | WVRQAPGKGLEWVS | RFTISRDNSKNTLYLQMNSLRAEDTAVYYCAK | WGRGTLVTVSS |
| | SLRLSCAASGFTFS | (SEQ ID NO: 291) | (SEQ ID NO: 299) | (SEQ ID NO: 307) |
| | (SEQ ID NO: 283) | | | |
| Ab17 | EVQLLESGGGLVQPGG | WVRQAPGKGLEWVS | RFTISRDNSKNTLYLQMNSLRAEDTATYYCAR | WGRGTLVTVSS |
| | SLRLSCAASGFTFS | (SEQ ID NO: 292) | (SEQ ID NO: 300) | (SEQ ID NO: 308) |
| | (SEQ ID NO: 284) | | | |
| Ab18 | EVQLLESGGGLVQPGG | WVRQAPGKGLEWVS | RFTISRDNSKNTLYLQMNSLRAEDTAVYYCAT | WGQGTMVTVSS |
| | SLRLSCAASGFTFS | (SEQ ID NO: 293) | (SEQ ID NO: 301) | (SEQ ID NO: 309) |
| | (SEQ ID NO: 285) | | | |
| Ab19 | EVQLLESGGGLVQPGG | WVRQAPGKGLEWVS | RFTISRDNSKNTLYLQMNSLRAEDTAVYYCAR | WGQGTMVTVSS |
| | SLRLSCAASGFTFS | (SEQ ID NO: 294) | (SEQ ID NO: 302) | (SEQ ID NO: 310) |
| | (SEQ ID NO: 286) | | | |
| Ab20 | EVQLLESGGGLVQPGG | WVRQAPGKGLEWVS | RFTISRDNSKNTLYLQMNSLRAEDTAVYYCAR | WGQGTLVTVSS |
| | SLRLSCAASGFTFS | (SEQ ID NO: 295) | (SEQ ID NO: 303) | (SEQ ID NO: 311) |
| | (SEQ ID NO: 287) | | | |
| Ab21 | EVQLLESGGGLVQPGG | WVRQAPGKGLEWVS | RFTISRDNSKNTLYLQMNSLRAEDTAVYYCAR | WGQGTMVTVSS |
| | SLRLSCAASGFTFS | (SEQ ID NO: 341) | (SEQ ID NO: 342) | (SEQ ID NO: 343) |
| | (SEQ ID NO: 340) | | | |

In certain aspects, an antibody described herein comprises one or more VL FRs having the amino acid sequence described herein (e.g., see Tables 2A and 2B), wherein the antibody immunospecifically binds to a KIT polypeptide, e.g., a human KIT polypeptide, for example, a D4/D5 region of KIT (e.g., human KIT), for example SEQ ID NO: 171. In specific embodiments, an antibody (e.g., human antibody) described herein comprises a VL chain region comprising FR1, CDR1, FR2, CDR2, FR3, CDR3, and FR4. In certain embodiments, the VL FR1 has the amino acid sequence of any one of SEQ ID NOs: 38-49, 224-231, and 333. In certain embodiments, the VL FR1 has the amino acid sequence of any one of SEQ ID NOs: 38-49 and 224-231 starting at the second amino acid residue, of SEQ ID NOs: 38-49 and 224-231, respectively. In some embodiments, the VL FR2 has the amino acid sequence of any one of SEQ ID NOs: 50-61, 232-239, and 334. In some embodiments, the VL FR3 has the amino acid sequence of any one of SEQ ID NOs: 62-73, 240-247, and 335. In some embodiments, the VL FR4 has the amino acid sequence of any one of SEQ ID NOs: 74-85, 258-255, and 336. In some embodiments, the VL FR4 has the amino acid sequence of any one of SEQ ID NOs: 74-85, 258-255, and 336, and further comprises an R (Arg) amino acid residue at the C-terminus. In certain embodiments, a VL FR4 has the amino acid sequence of any one of the VL FR4 sequences described in Tables 2A and 2B, and further comprises an R (Arg) amino acid residue at the C-terminus.

In certain embodiments, an anti-KIT antibody described herein comprises one or more VH FRs having the amino acid sequence described herein (e.g., see Tables 4A and 4B). In specific embodiments, an antibody (e.g., human antibody) comprises a VH chain region comprising FR1, CDR1, FR2, CDR2, FR3, CDR3, and FR4, respectively. In some embodiments, the VH FR1 has the amino acid sequence of any one of SEQ ID NOs: 122-133, 280-287, and 340. In some embodiments, the VH FR2 has the amino acid sequence of any one of SEQ ID NOs: 134-145, 288-295, and 341. In some embodiments, the VH FR3 has the amino acid sequence of any one of SEQ ID NO: 146-157, 296-303, and 342. In some embodiments, the VH FR4 has the amino acid sequence of any one of SEQ ID NO: 158-169, 304-311, and 343.

In a particular embodiment, an antibody described herein, comprises a VH FR1 having the amino acid sequence of SEQ ID NO: 122. In a particular embodiment, an antibody described herein, comprises a VH FR1 having the amino acid sequence of SEQ ID NO: 134. In a particular embodiment, an antibody described herein, comprises a VH FR1 having the amino acid sequence of SEQ ID NO: 122, a VH FR2 having the amino acid sequence of SEQ ID NO: 134, a VH FR3 having the amino acid sequence of any one of SEQ ID NOs: 146-157, and a VH FR4 having the amino acid sequence of any one of SEQ ID NOs: 158-169, optionally further comprising an R (Arg) amino acid residue at the C-terminus.

In another specific embodiment, an antibody described herein comprises (i) a VL chain region comprising VL FR1, VL FR2, VL FR3, and VL FR4 having the amino acid sequences of SEQ ID NO: 38 (Ab1), SEQ ID NO: 50 (Ab1), SEQ ID NO: 62 (Ab1), and SEQ ID NO: 74 (Ab1), respectively; and (ii) a VH chain region comprising VH FR1, VH FR2, VH FR3, and VH FR4 having the amino acid sequences of SEQ ID NO: 122 (Ab1), SEQ ID NO: 134 (Ab1), SEQ ID NO: 146 (Ab1), and SEQ ID NO: 158 (Ab1), respectively. In a certain embodiment, an antibody described herein comprises (i) a VL chain region comprising VL FR1 having the amino acid sequence of SEQ ID NO: 38 (Ab1) starting at the second amino acid residue of amino acid residue of SEQ ID NO: 38, and/or comprising a VL FR4 having the amino acid sequence of SEQ ID NO: 74 (Ab1) further comprising an R (Arg) amino acid residue at the C-terminus.

In another specific embodiment, an antibody described herein comprises (i) a VL chain region comprising VL FR1, VL FR2, VL FR3, and VL FR4 having the amino acid sequences of SEQ ID NO: 333 (Ab21) or SEQ ID NO: 38 starting at the second amino acid residue of SEQ ID NO: 38; SEQ ID NO: 50 (Ab1); SEQ ID NO: 62 (Ab1); and SEQ ID NO: 74 (Ab1), respectively; and (ii) a VH chain region comprising VH FR1, VH FR2, VH FR3, and VH FR4 having the amino acid sequences of SEQ ID NO: 122 (Ab1), SEQ ID NO: 134 (Ab1), SEQ ID NO: 146 (Ab1), and SEQ ID NO: 158 (Ab1), respectively. In a certain embodiment, the VL FR4 having the amino acid sequence of SEQ ID NO: 74 (Ab1) further comprises an R (Arg) amino acid residue at the C-terminus.

In another specific embodiment, an antibody described herein comprises (i) a VL chain region comprising VL FR1, VL FR2, VL FR3, and VL FR4 having the amino acid sequences of SEQ ID NO: 39 (Ab2), SEQ ID NO: 51 (Ab2), SEQ ID NO: 63 (Ab2), and SEQ ID NO: 75 (Ab2), respectively; and (ii) a VH chain region comprising VH FR1, VH FR2, VH FR3, and VH FR4 having the amino acid sequences of SEQ ID NO: 123 (Ab2), SEQ ID NO: 135 (Ab2), SEQ ID NO: 147 (Ab2), and SEQ ID NO: 159 (Ab2), respectively.

In another specific embodiment, an antibody described herein comprises (i) a VL chain region comprising VL FR1, VL FR2, VL FR3, and VL FR4 having the amino acid sequences of SEQ ID NO: 333 (Ab21), SEQ ID NO: 334 (Ab21), SEQ ID NO: 335 (Ab21), and SEQ ID NO: 336 (Ab21), respectively; and (ii) a VH chain region comprising VH FR1, VH FR2, VH FR3, and VH FR4 having the amino acid sequences of SEQ ID NO: 340 (Ab21), SEQ ID NO: 341 (Ab21), SEQ ID NO: 342 (Ab21), and SEQ ID NO: 343 (Ab21), respectively. In a particular embodiment, the VL FR4 having the amino acid sequence of SEQ ID NO: 336 further comprises an R (Arg) amino acid residue at the C-terminus.

In specific embodiments, an antibody described herein, which immunospecifically bind to a KIT polypeptide, e.g., a human KIT polypeptide, for example, a D4/D5 region of KIT (e.g., human KIT, for example SEQ ID NO: 171), comprises framework regions (e.g., framework regions of the VL domain and VH domain) that are human framework regions. Non-limiting examples of human framework regions are described in the art, e.g., see Kabat et al. (1991) Sequences of Proteins of Immunological Interest, Fifth Edition, U.S. Department of Health and Human Services, NIH Publication No. 91-3242).

In specific aspects, provided herein is an antibody comprising an antibody light chain and heavy chain, e.g., a separate light chain and heavy chain. With respect to the light chain, in a specific embodiment, the light chain of an antibody described herein is a kappa light chain. In another specific embodiment, the light chain of an antibody described herein is a lambda light chain. In yet another specific embodiment, the light chain of an antibody described herein is a human kappa light chain or a human lambda light chain. In a particular embodiment, an antibody described herein, which immunospecifically binds to a KIT polypeptide (e.g., a KIT polypeptide comprising a D4/D5 region of KIT, for example human KIT (e.g., SEQ ID NO: 171)) comprises a light chain wherein the amino acid sequence of the VL chain region can comprise any amino acid sequence described herein (e.g., any one of SEQ ID NOs: 176-187 and 350), and wherein the constant region of the light chain comprises the amino acid sequence of a human kappa light chain constant region. In another particular embodiment, an antibody described herein, which immunospecifically binds to a KIT polypeptide (e.g., a KIT polypeptide comprising a KIT polypeptide comprising a D4/D5 region of KIT, for example human KIT (e.g., SEQ ID NO: 171)) comprises a light chain wherein the amino acid sequence of the VL chain region can comprise any amino acid sequence described herein (e.g., any one of SEQ ID NOs: 176-187 and 350), and wherein the constant region of the light chain comprises the amino acid sequence of a human lambda light chain constant region. Non-limiting examples of human constant region sequences have been described in the art, e.g., see U.S. Pat. No. 5,693,780 and Kabat et al. (1991) Sequences of Proteins of Immunological Interest, Fifth Edition, U.S. Department of Health and Human Services, NIH Publication No. 91-3242.

In a particular embodiment, an antibody described herein, which immunospecifically binds to a KIT polypeptide (e.g., a KIT polypeptide comprising a KIT polypeptide comprising a D4/D5 region of KIT, for example human KIT (e.g., SEQ ID NO: 171)), comprises a light chain comprising a constant region having the amino acid sequence of SEQ ID NO: 344 (RTVA APSVFIFPPS DEQLKSGTAS VVCLLNNFYP REAKVQWKVD NALQSGNSQE SVTEQDSKDS TYSLSSTLTL SKADYEKHKV YACEVTHQGL SSPVTKSFNR GEC). In a particular embodiment, an antibody described herein, which immunospecifically binds to a KIT polypeptide (e.g., a KIT polypeptide comprising a KIT polypeptide comprising a D4/D5 region of KIT, for example human KIT (e.g., SEQ ID NO: 171)), comprises a light chain comprising a constant region having the amino acid sequence of SEQ ID NO: 965 (TVA APSVFIFPPS DEQLKSGTAS VVCLLNNFYP REAKVQWKVD NALQSGNSQE SVTEQDSKDS TYSLSSTLTL SKADYEKHKV YACEVTHQGL SSPVTKSFNR GEC).

With respect to the heavy chain, in a specific embodiment, the heavy chain of an antibody described herein can be an alpha (α), delta (δ), epsilon (ε), gamma (γ) or mu (μ) heavy chain. In another specific embodiment, the heavy chain of an antibody described can comprise a human alpha (α), delta (δ), epsilon (ε), gamma (γ) or mu (μ) heavy chain. In a particular embodiment, an antibody described herein, which immunospecifically binds to a KIT polypeptide (e.g., a KIT polypeptide comprising a KIT polypeptide comprising a D4/D5 region of KIT, for example human KIT (e.g., SEQ ID NO: 171)), comprises a heavy chain wherein the amino acid sequence of the VH chain region can comprise any amino acid sequence described herein (e.g., any of SEQ ID NOs: 188-199), and wherein the constant region of the heavy chain comprises the amino acid sequence of a human gamma (γ) heavy chain constant region. Non-limiting examples of human constant region sequences have been described in the art, e.g., see U.S. Pat. No. 5,693,780 and Kabat et al. (1991) Sequences of Proteins of Immunological Interest, Fifth Edition, U.S. Department of Health and Human Services, NIH Publication No. 91-3242.

In a particular embodiment, an antibody described herein, which immunospecifically binds to a KIT polypeptide (e.g., a KIT polypeptide comprising a KIT polypeptide comprising a D4/D5 region of KIT, for example human KIT (e.g., SEQ ID NO: 171)), comprises a heavy chain comprising a constant region having the amino acid sequence of SEQ ID NO: 345 (ASTKGPSVF PLAPSSKSTS GGTAALGCLV KDYFPEPVTV SWNSGALTSG VHTFPAVLQS SGLYSLSSVV TVPSSSLGTQ TYICNVNHKP SNTKVDKKVE PKSCDKTHTC PPCPAPELLG GPSVFLFPPK PKDTLMISRT PEVTCVVVDV SHEDPEVKFN WYVDGVEVHN AKTKPREEQY NSTYRVVSVL TVLHQDWLNG KEYKCKVSNK ALPAPIEKTI SKAKGQPREP QVYTLPPSRD ELTKNQVSLT CLVKGFYPSD IAVEWESNGQ PENNYKTTPP VLDSDGSFFL YSKLTVDKSR WQQGNVFSCS VMHEALHNHY TQKSLSLSPG K).

In a specific embodiment, an antibody described herein, which immunospecifically binds to a KIT polypeptide (e.g., a D4/D5 region of KIT, for example human KIT) comprises a VL chain region and a VH chain region comprising any amino acid sequences described herein, and wherein the constant regions comprise the amino acid sequences of the constant regions of an IgG, IgE, IgM, IgD, IgA or IgY immunoglobulin molecule, or a human IgG, IgE, IgM, IgD, IgA or IgY immunoglobulin molecule. In another specific embodiment, an antibody described herein, which immunospecifically binds to a KIT polypeptide (e.g., a D4/D5 region of KIT, for example human KIT) comprises a VL chain region and a VH chain region comprising any amino acid sequences described herein, and wherein the constant regions comprise the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. In a particular embodiment, the constant regions comprise the amino acid sequences of the constant regions of a human IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule.

In yet another specific embodiment, an antibody described herein, which immunospecifically binds to a KIT polypeptide (e.g., a D4/D5 region of KIT, for example human KIT), comprises a VL chain region and a VH chain region comprising any amino acid sequences described herein, and wherein the constant regions comprise the amino acid sequences of the constant regions of a human IgG1 (e.g., isotype a, z, or f) or human IgG4. In a particular embodiment, an antibody described herein, which immunospecifically binds to a KIT polypeptide (e.g., a D4/D5 region of KIT, for example human KIT) comprises a VL chain region and a VH chain region comprising any amino acid sequences described herein, and wherein the constant regions comprise the amino acid sequences of the constant region of a human IgG1 (isotype f). Non-limiting examples of human constant regions are described in the art, e.g., see Kabat et al. (1991) Sequences of Proteins of Immunological Interest, Fifth Edition, U.S. Department of Health and Human Services, NIH Publication No. 91-3242. In another specific embodiment, an antibody described herein, which immunospecifically binds to a KIT polypeptide (e.g., a D4/D5 region of KIT, for example human KIT), comprises a VL chain region and a VH chain region comprising any amino acid sequences described herein, and wherein the antibody further comprises (i) a light chain constant region comprising the amino acid sequence of SEQ ID NO: 344, and (ii) a heavy chain constant region comprising the amino acid sequence of SEQ ID NO: 345. In another specific embodiment, an antibody described herein, which immunospecifically binds to a KIT polypeptide (e.g., a D4/D5 region of KIT, for example human KIT), comprises a VL chain region and a VH chain region comprising any amino acid sequences described herein, and wherein the antibody further comprises (i) a light chain constant region comprising the amino acid sequence of SEQ ID NO: 965, and (ii) a heavy chain constant region comprising the amino acid sequence of SEQ ID NO: 345.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising a VL CDR1, VL CDR2, and VL CDR3 having the amino acid sequences of SEQ ID NO: 2 (VL CDR1 of Ab1), SEQ ID NO: 14 (VL CDR2 of Ab1), and SEQ ID NO: 26 (VL CDR3 of Ab1), respectively; (ii) the heavy chain comprises a VH chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain. In particular embodiments, the constant light chain domain comprises the amino acid sequence of SEQ ID NO: 344 or 965; and the constant heavy chain domain comprises the amino acid sequence of SEQ ID NO: 345.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising a VL CDR1, VL CDR2, and VL CDR3 having the amino acid sequences of SEQ ID NO: 3 (VL CDR1 of Ab2), SEQ ID NO: 15 (VL CDR2 of Ab2), and SEQ ID NO: 27 (VL CDR3 of Ab2), respectively; (ii) the heavy chain comprises a VH chain region comprising a VH CDR1, VH CDR2, and VH CDR3 having the amino acid sequences of SEQ ID NO: 87 (VH CDR1 of Ab2), SEQ ID NO: 99 (VH CDR2 of Ab2), and SEQ ID NO: 111 (VH CDR3 of Ab2), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain. In particular embodiments, the constant light chain domain comprises the amino acid sequence of SEQ ID NO: 344 or 965; and the constant heavy chain domain comprises the amino acid sequence of SEQ ID NO: 345.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising a VL CDR1, VL CDR2, and VL CDR3 having the amino acid sequences of SEQ ID NO: 4 (VL CDR1 of Ab3), SEQ ID NO: 16 (VL CDR2 of Ab3), and SEQ ID NO: 28 (VL CDR3 of Ab3), respectively; (ii) the heavy chain comprises a VH chain region comprising a VH CDR1, VH CDR2, and VH CDR3 having the amino acid sequences of SEQ ID NO: 88 (VH CDR1 of Ab3), SEQ ID NO: 100 (VH CDR2 of Ab3), and SEQ ID NO: 112 (VH CDR3 of Ab3), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain. In particular embodiments, the constant light chain domain comprises the amino acid sequence of SEQ ID NO: 344 or 965; and the constant heavy chain domain comprises the amino acid sequence of SEQ ID NO: 345.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising a VL CDR1, VL CDR2, and VL CDR3 having the amino acid sequences of SEQ ID NO: 5 (VL CDR1 of Ab4), SEQ ID NO: 17 (VL CDR2 of Ab4), and SEQ ID NO: 29 (VL CDR3 of Ab4), respectively; (ii) the heavy chain comprises a VH chain region comprising a VH CDR1, VH CDR2, and VH CDR3 having the amino acid sequences of SEQ ID NO: 89 (VH CDR1 of Ab4), SEQ ID NO: 101 (VH CDR2 of Ab4), and SEQ ID NO: 113 (VH CDR3 of Ab4), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising a VL CDR1, VL CDR2, and VL CDR3 having the amino acid sequences of SEQ ID NO: 6 (VL CDR1 of Ab5), SEQ ID NO: 18 (VL CDR2 of Ab5), and SEQ ID NO: 30 (VL CDR3 of Ab5), respectively; (ii) the heavy chain comprises a VH chain region comprising a VH CDR1, VH CDR2, and VH CDR3 having the amino acid sequences of SEQ ID NO: 90 (VH CDR1 of Ab5), SEQ ID NO: 102 (VH CDR2 of Ab5), and SEQ ID NO: 114 (VH CDR3 of Ab5), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising a VL CDR1, VL CDR2, and VL CDR3 having the amino acid sequences of SEQ ID NO: 7 (VL CDR1 of Ab6), SEQ ID NO: 19 (VL CDR2 of Ab6), and SEQ ID NO: 31 (VL CDR3 of Ab6), respectively; (ii) the heavy chain comprises a VH chain region comprising a VH CDR1, VH CDR2, and VH CDR3 having the amino acid sequences of SEQ ID NO: 91 (VH CDR1 of Ab6), SEQ ID NO: 103 (VH CDR2 of Ab6), and SEQ ID NO: 115 (VH CDR3 of Ab6), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising a VL CDR1, VL CDR2, and VL CDR3 having the amino acid sequences of SEQ ID NO: 8 (VL CDR1 of Ab7), SEQ ID NO: 20 (VL CDR2 of Ab7), and SEQ ID NO: 32 (VL CDR3 of Ab7), respectively; (ii) the heavy chain comprises a VH chain region comprising a VH CDR1, VH CDR2, and VH CDR3 having the amino acid sequences of SEQ ID NO: 92 (VH CDR1 of Ab7), SEQ ID NO: 104 (VH CDR2 of Ab7), and SEQ ID NO: 116 (VH CDR3 of Ab7), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising a VL CDR1, VL CDR2, and VL CDR3 having the amino acid sequences of SEQ ID NO: 9 (VL CDR1 of Ab8), SEQ ID NO: 21 (VL CDR2 of Ab8), and SEQ ID NO: 33 (VL CDR3 of Ab8), respectively; (ii) the heavy chain comprises a VH chain region comprising a VH CDR1, VH CDR2, and VH CDR3 having the amino acid sequences of SEQ ID NO: 93 (VH CDR1 of Ab8), SEQ ID NO: 105 (VH CDR2 of Ab8), and SEQ ID NO: 117 (VH CDR3 of Ab8), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising a VL CDR1, VL CDR2, and VL CDR3 having the amino acid sequences of SEQ ID NO: 10 (VL CDR1 of Ab9), SEQ ID NO: 22 (VL CDR2 of Ab9), and SEQ ID NO: 34 (VL CDR3 of Ab9), respectively; (ii) the heavy chain comprises a VH chain region comprising a VH CDR1, VH CDR2, and VH CDR3 having the amino acid sequences of SEQ ID NO: 94 (VH CDR1 of Ab9), SEQ ID NO: 106 (VH CDR2 of Ab9), and SEQ ID NO: 118 (VH CDR3 of Ab9), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising a VL CDR1, VL CDR2, and VL CDR3 having the amino acid sequences of SEQ ID NO: 11 (VL CDR1 of Ab10), SEQ ID NO: 23 (VL CDR2 of Ab10), and SEQ ID NO: 35 (VL CDR3 of Ab10), respectively; (ii) the heavy chain comprises a VH chain region comprising a VH CDR1, VH CDR2, and VH CDR3 having the amino acid sequences of SEQ ID NO: 95 (VH CDR1 of Ab10), SEQ ID NO: 107 (VH CDR2 of Ab10), and SEQ ID NO: 119 (VH CDR3 of Ab10), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising a VL CDR1, VL CDR2, and VL CDR3 having the amino acid sequences of SEQ ID NO: 12 (VL CDR1 of Ab11), SEQ ID NO: 24 (VL CDR2 of Ab11), and SEQ ID NO: 36 (VL CDR3 of Ab11), respectively; (ii) the heavy chain comprises a VH chain region comprising a VH CDR1, VH CDR2, and VH CDR3 having the amino acid sequences of SEQ ID NO: 96 (VH CDR1 of Ab11), SEQ ID NO: 108 (VH CDR2 of Ab11), and SEQ ID NO: 120 (VH CDR3 of Ab11), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising a VL CDR1, VL CDR2, and VL CDR3 having the amino acid sequences of SEQ ID NO: 13 (VL CDR1 of Ab12), SEQ ID NO: 25 (VL CDR2 of Ab12), and SEQ ID NO: 37 (VL CDR3 of Ab12), respectively; (ii) the heavy chain comprises a VH chain region comprising a VH CDR1, VH CDR2, and VH CDR3 having the amino acid sequences of SEQ ID NO: 97 (VH CDR1 of Ab12), SEQ ID NO: 109 (VH CDR2 of Ab12), and SEQ ID NO: 121 (VH CDR3 of Ab12), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising a VL CDR1, VL CDR2, and VL CDR3 having the amino acid sequences of SEQ ID NO: 330 (VL CDR1 of Ab21), SEQ ID NO: 331 (VL CDR2 of Ab21), and SEQ ID NO: 332 (VL CDR3 of Ab21), respectively; (ii) the heavy chain comprises a VH chain region comprising a VH CDR1, VH CDR2, and VH CDR3 having the amino acid sequences of SEQ ID NO: 337 (VH CDR1 of Ab21), SEQ ID NO: 338 (VH CDR2 of Ab21), and SEQ ID NO: 339 (VH CDR3 of Ab21), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain. In particular embodiments, the constant light chain domain comprises the amino acid sequence of SEQ ID NO: 344 or 965; and the constant heavy chain domain comprises the amino acid sequence of SEQ ID NO: 345.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising the amino acid sequence of SEQ ID NO: 176 (VL domain of Ab1); (ii) the heavy chain comprises a VH chain region comprising the amino acid sequence of SEQ ID NO: 188 (VH domain of Ab1); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain. In particular embodiments, the constant light chain domain comprises the amino acid sequence of SEQ ID NO: 344; and the constant heavy chain domain comprises the amino acid sequence of SEQ ID NO: 345. In certain embodiments, the light chain comprises a VL chain region comprising the amino acid sequence of SEQ ID NO: 176 (VL domain of Ab1) starting at the second amino acid residue of SEQ ID NO: 176. In certain embodiments, the light chain comprises a VL chain region comprising the amino acid sequence of SEQ ID NO: 350 (VL domain of Ab1). In certain embodiments, the light chain comprises a VL chain region comprising the amino acid sequence of SEQ ID NO: 350 (VL domain of Ab1), and the constant light chain domain comprises the amino acid sequence of SEQ ID NO: 344. In a certain embodiment, the light chain comprises a VL chain region comprising the amino acid sequence of SEQ ID NO: 176 or 350, wherein the VL chain region further comprises the amino acid R (Arg) at the C-terminus. In a certain embodiment, the light chain comprises a VL chain region comprising the amino acid sequence of SEQ ID NO: 176 or 350, wherein the VL chain region further comprises the amino acid R (Arg) at the C-terminus, and the constant light chain domain comprises the amino acid sequence of SEQ ID NO: 965. In certain embodiments, the light chain comprises a VL chain region comprising the amino acid sequence of SEQ ID NO: 963 (VL domain of Ab1), and a light chain constant region comprising the amino acid sequence of SEQ ID NO: 965.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising the amino acid sequence of SEQ ID NO: 177 (VL domain of Ab2); (ii) the heavy chain comprises a VH chain region comprising the amino acid sequence of SEQ ID NO: 189 (VH domain of Ab2); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising the amino acid sequence of SEQ ID NO: 178 (VL domain of Ab3); (ii) the heavy chain comprises a VH chain region comprising the amino acid sequence of SEQ ID NO: 190 (VH domain of Ab3); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising the amino acid sequence of SEQ ID NO: 179 (VL domain of Ab4); (ii) the heavy chain comprises a VH chain region comprising the amino acid sequence of SEQ ID NO: 191 (VH domain of Ab4); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising the amino acid sequence of SEQ ID NO: 180 (VL domain of Ab5); (ii) the heavy chain comprises a VH chain region comprising the amino acid sequence of SEQ ID NO: 192 (VH domain of Ab5); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising the amino acid sequence of SEQ ID NO: 181 (VL domain of Ab6); (ii) the heavy chain comprises a VH chain region comprising the amino acid sequence of SEQ ID NO: 193 (VH domain of Ab6); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising the amino acid sequence of SEQ ID NO: 182 (VL domain of Ab7); (ii) the heavy chain comprises a VH chain region comprising the amino acid sequence of SEQ ID NO: 194 (VH domain of Ab7); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising the amino acid sequence of SEQ ID NO: 183 (VL domain of Ab8); (ii) the heavy chain comprises a VH chain region comprising the amino acid sequence of SEQ ID NO: 195 (VH domain of Ab8); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising the amino acid sequence of SEQ ID NO: 184 (VL domain of Ab9); (ii) the heavy chain comprises a VH chain region comprising the amino acid sequence of SEQ ID NO: 196 (VH domain of Ab9); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising the amino acid sequence of SEQ ID NO: 185 (VL domain of Ab10); (ii) the heavy chain comprises a VH chain region comprising the amino acid sequence of SEQ ID NO: 197 (VH domain of Ab10); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising the amino acid sequence of SEQ ID NO: 186 (VL domain of Ab11); (ii) the heavy chain comprises a VH chain region comprising the amino acid sequence of SEQ ID NO: 198 (VH domain of Ab11); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising the amino acid sequence of SEQ ID NO: 187 (VL domain of Ab12); (ii) the heavy chain comprises a VH chain region comprising the amino acid sequence of SEQ ID NO: 199 (VH domain of Ab12); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising the amino acid sequence of SEQ ID NO: 328 (VL domain of Ab21); (ii) the heavy chain comprises a VH chain region comprising the amino acid sequence of SEQ ID NO: 329 (VH domain of Ab21); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain. In particular embodiments, the constant light chain domain comprises the amino acid sequence of SEQ ID NO: 344; and the constant heavy chain domain comprises the amino acid sequence of SEQ ID NO: 345. In particular embodiments, the constant light chain domain comprises the amino acid sequence of SEQ ID NO: 965; and the constant heavy chain domain comprises the amino acid sequence of SEQ ID NO: 345. In certain embodiments, the VL chain region comprises the amino acid sequence of SEQ ID NO: 964 and a light chain constant region comprising the amino acid sequence of SEQ ID NO: 965. In a particular embodiment, the light chain comprises a VL chain region comprising the amino acid sequence of SEQ ID NO: 328 (VL domain of Ab21), and a constant light chain domain comprising the amino acid sequence of SEQ ID NO: 344. In a particular embodiment, the light chain comprises a VL chain region comprising the amino acid sequence of SEQ ID NO: 328 (VL domain of Ab21), which further comprises an R (Arg) at the C-terminus, and a constant light chain domain comprising the amino acid sequence of SEQ ID NO: 965.

In another particular embodiment, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising the amino acid sequence of any one of SEQ ID NOs: 312-319; (ii) the heavy chain comprises a VH chain region comprising the amino acid sequence of any one of SEQ ID NOs: 320-327; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain. In a particular embodiment, the VL chain region comprises the amino acid sequence of any one of SEQ ID NOs: 312-319 starting at the second amino acid position, and/or the VL chain region further comprises an R (Arg) at the C-terminus.

In certain embodiments, with respect to any of these antibodies described herein, the VL chain region further comprises human framework regions; and the VH chain region further comprises human framework regions.

In certain embodiments, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising human framework regions; (ii) the heavy chain comprises a VH chain region comprising human framework regions; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In certain embodiments, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising VL FR1, VL FR2, VL FR3, and VL FR4 having the amino acid sequence of SEQ ID NO: 38 (Ab1), SEQ ID NO: 50 (Ab1), SEQ ID NO: 62 (Ab1), and SEQ ID NO: 74 (Ab1), respectively; (ii) the heavy chain comprises a VH chain region comprising VH FR1, VH FR2, VH FR3, and VH FR4 having the amino acid sequence of SEQ ID NO: 122 (Ab1), SEQ ID NO: 134 (Ab1), SEQ ID NO: 146 (Ab1), and SEQ ID NO: 158 (Ab1), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain. In a particular embodiment, the VL FR1 has the amino acid sequence of SEQ ID NO: 38 starting with the second amino acid residue of SEQ ID NO: 38. In a particular embodiment, the VL FR4, which has the amino acid sequence of SEQ ID NO: 74, further comprises an R (Arg) amino acid residue at the C-terminus. In particular embodiments, the constant light chain domain of these antibodies comprises the amino acid sequence of SEQ ID NO: 344; and the constant heavy chain domain of these antibodies comprises the amino acid sequence of SEQ ID NO: 345. In particular embodiments, the constant light chain domain of these antibodies comprises the amino acid sequence of SEQ ID NO: 965; and the constant heavy chain domain of these antibodies comprises the amino acid sequence of SEQ ID NO: 345.

In certain embodiments, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising VL FR1, VL FR2, VL FR3, and VL FR4 having the amino acid sequence of SEQ ID NO: 39 (Ab2), SEQ ID NO: 51 (Ab2), SEQ ID NO: 63 (Ab2), and SEQ ID NO: 75 (Ab2), respectively; (ii) the heavy chain comprises a VH chain region comprising VH FR1, VH FR2, VH FR3, and VH FR4 having the amino acid sequence of SEQ ID NO: 123 (Ab2), SEQ ID NO: 135 (Ab2), SEQ ID NO: 147 (Ab2), and SEQ ID NO: 159 (Ab2), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In certain embodiments, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising VL FR1, VL FR2, VL FR3, and VL FR4 having the amino acid sequence of SEQ ID NO: 40 (Ab3), SEQ ID NO: 52 (Ab3), SEQ ID NO: 64 (Ab3), and SEQ ID NO: 76 (Ab3), respectively; (ii) the heavy chain comprises a VH chain region comprising VH FR1, VH FR2, VH FR3, and VH FR4 having the amino acid sequence of SEQ ID NO: 124 (Ab3), SEQ ID NO: 136 (Ab3), SEQ ID NO: 148 (Ab3), and SEQ ID NO: 160 (Ab3), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In certain embodiments, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising VL FR1, VL FR2, VL FR3, and VL FR4 having the amino acid sequence of SEQ ID NO: 41 (Ab4), SEQ ID NO: 53 (Ab4), SEQ ID NO: 65 (Ab4), and SEQ ID NO: 77 (Ab4), respectively; (ii) the heavy chain comprises a VH chain region comprising VH FR1, VH FR2, VH FR3, and VH FR4 having the amino acid sequence of SEQ ID NO: 125 (Ab4), SEQ ID NO: 137 (Ab4), SEQ ID NO: 149 (Ab4), and SEQ ID NO: 161 (Ab4), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In certain embodiments, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising VL FR1, VL FR2, VL FR3, and VL FR4 having the amino acid sequence of SEQ ID NO: 42 (Ab5), SEQ ID NO: 54 (Ab5), SEQ ID NO: 66 (Ab5), and SEQ ID NO: 78 (Ab5), respectively; (ii) the heavy chain comprises a VH chain region comprising VH FR1, VH FR2, VH FR3, and VH FR4 having the amino acid sequence of SEQ ID NO: 126 (Ab5), SEQ ID NO: 138 (Ab5), SEQ ID NO: 150 (Ab5), and SEQ ID NO: 162 (Ab5), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In certain embodiments, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising VL FR1, VL FR2, VL FR3, and VL FR4 having the amino acid sequence of SEQ ID NO: 43 (Ab6), SEQ ID NO: 55 (Ab6), SEQ ID NO: 67 (Ab6), and SEQ ID NO: 79 (Ab6), respectively; (ii) the heavy chain comprises a VH chain region comprising VH FR1, VH FR2, VH FR3, and VH FR4 having the amino acid sequence of SEQ ID NO: 127 (Ab6), SEQ ID NO: 139 (Ab6), SEQ ID NO: 151 (Ab6), and SEQ ID NO: 163 (Ab6), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In certain embodiments, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising VL FR1, VL FR2, VL FR3, and VL FR4 having the amino acid sequence of SEQ ID NO: 44 (Ab7), SEQ ID NO: 56 (Ab7), SEQ ID NO: 68 (Ab7), and SEQ ID NO: 80 (Ab7), respectively; (ii) the heavy chain comprises a VH chain region comprising VH FR1, VH FR2, VH FR3, and VH FR4 having the amino acid sequence of SEQ ID NO: 128 (Ab7), SEQ ID NO: 140 (Ab7), SEQ ID NO: 152 (Ab7), and SEQ ID NO: 164 (Ab7), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In certain embodiments, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising VL FR1, VL FR2, VL FR3, and VL FR4 having the amino acid sequence of SEQ ID NO: 45 (Ab8), SEQ ID NO: 57 (Ab8), SEQ ID NO: 69 (Ab8), and SEQ ID NO: 81 (Ab8), respectively; (ii) the heavy chain comprises a VH chain region comprising VH FR1, VH FR2, VH FR3, and VH FR4 having the amino acid sequence of SEQ ID NO: 129 (Ab8), SEQ ID NO: 141 (Ab8), SEQ ID NO: 153 (Ab8), and SEQ ID NO: 165 (Ab8), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In certain embodiments, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising VL FR1, VL FR2, VL FR3, and VL FR4 having the amino acid sequence of SEQ ID NO: 46 (Ab9), SEQ ID NO: 58 (Ab9), SEQ ID NO: 70 (Ab9), and SEQ ID NO: 82 (Ab9), respectively; (ii) the heavy chain comprises a VH chain region comprising VH FR1, VH FR2, VH FR3, and VH FR4 having the amino acid sequence of SEQ ID NO: 130 (Ab9), SEQ ID NO: 142 (Ab9), SEQ ID NO: 154 (Ab9), and SEQ ID NO: 166 (Ab9), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In certain embodiments, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising VL FR1, VL FR2, VL FR3, and VL FR4 having the amino acid sequence of SEQ ID NO: 47 (Ab10), SEQ ID NO: 59 (Ab10), SEQ ID NO: 71 (Ab10), and SEQ ID NO: 83 (Ab10), respectively; (ii) the heavy chain comprises a VH chain region comprising VH FR1, VH FR2, VH FR3, and VH FR4 having the amino acid sequence of SEQ ID NO: 131 (Ab10), SEQ ID NO: 143 (Ab10), SEQ ID NO: 155 (Ab10), and SEQ ID NO: 167 (Ab10), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In certain embodiments, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising VL FR1, VL FR2, VL FR3, and VL FR4 having the amino acid sequence of SEQ ID NO: 48 (Ab11), SEQ ID NO: 60 (Ab11), SEQ ID NO: 72 (Ab11), and SEQ ID NO: 84 (Ab11), respectively; (ii) the heavy chain comprises a VH chain region comprising VH FR1, VH FR2, VH FR3, and VH FR4 having the amino acid sequence of SEQ ID NO: 132 (Ab11), SEQ ID NO: 144 (Ab11), SEQ ID NO: 156 (Ab11), and SEQ ID NO: 168 (Ab11), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In certain embodiments, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising VL FR1, VL FR2, VL FR3, and VL FR4 having the amino acid sequence of SEQ ID NO: 49 (Ab12), SEQ ID NO: 61 (Ab12), SEQ ID NO: 73 (Ab12), and SEQ ID NO: 85 (Ab12), respectively; (ii) the heavy chain comprises a VH chain region comprising VH FR1, VH FR2, VH FR3, and VH FR4 having the amino acid sequence of SEQ ID NO: 133 (Ab12), SEQ ID NO: 145 (Ab12), SEQ ID NO: 157 (Ab12), and SEQ ID NO: 169 (Ab12), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In certain embodiments, an antibody described herein, which immunospecifically binds to a KIT polypeptide (e.g., a D4/D5 region of KIT, for example human KIT), comprises a light chain and a heavy chain, wherein (i) the light chain comprises a VL chain region comprising VL FR1, VL FR2, VL FR3, and VL FR4 having the amino acid sequence of SEQ ID NO: 333 (Ab21), SEQ ID NO: 334 (Ab21), SEQ ID NO: 335 (Ab21), and SEQ ID NO: 336 (Ab21), respectively; (ii) the heavy chain comprises a VH chain region comprising VH FR1, VH FR2, VH FR3, and VH FR4 having the amino acid sequence of SEQ ID NO: 340 (Ab21), SEQ ID NO: 341 (Ab21), SEQ ID NO: 342 (Ab21), and SEQ ID NO: 343 (Ab21), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain. In particular embodiments, the constant light chain domain of these antibodies comprises the amino acid sequence of SEQ ID NO: 344; and the constant heavy chain domain of these antibodies comprises the amino acid sequence of SEQ ID NO: 345. In a particular embodiment, the VL FR4, which has the amino acid sequence of SEQ ID NO: 336, further comprises an R (Arg) amino acid residue at the C-terminus. In particular embodiments, the constant light chain domain of these antibodies comprises the amino acid sequence of SEQ ID NO: 965; and the constant heavy chain domain of these antibodies comprises the amino acid sequence of SEQ ID NO: 345.

In certain aspects, also provided herein are antibodies, which immunospecifically binds to a KIT polypeptide (e.g., a D4/D5 region of KIT, for example human KIT), comprising one or more amino acid residue substitutions, e.g., in the VL chain region or VH chain region, for example, the CDRs or FRs.

In a specific embodiment, included herein is any antibody described herein comprising no more than 1, 2, 3, 4, 5, 6, 7, 8, 9, 10, 11, 12, 13, 14, 15, 16, 17, 18, 19, 20, 25, 30, 35, 40, 45, or 50 amino acid substitutions, deletions, or additions relative to its depicted sequence, wherein the resulting antibody immunospecifically binds to a KIT polypeptide (e.g., a D4/D5 region of KIT, for example human KIT). In a specific embodiment, an antibody described herein, which immunospecifically binds to a KIT polypeptide (e.g., a D4/D5 region of KIT, for example human KIT), comprises a light chain and/or a heavy chain comprising no more than 1, 2, 3, 4, 5, 6, 7, 8, 9, 10, 11, 12, 13, 14, 15, 16, 17, 18, 19, 20, 25, 30, 35, 40, 45, or 50 amino acid substitutions, deletions, or additions. In another specific embodiment, an antibody described herein, which immunospecifically binds to a KIT polypeptide (e.g., a D4/D5 region of KIT, for example human KIT), comprises a VL chain region and/or a VH chain region comprising no more than 1, 2, 3, 4, 5, 6, 7, 8, 9, 10, 11, 12, 13, 14, 15, 16, 17, 18, 19, 20, 25, or amino acid substitutions, deletions, or additions. In yet another specific embodiment, an antibody described herein, which immunospecifically binds to a KIT polypeptide (e.g., a D4/D5 region of KIT, for example human KIT), comprises one or more VL CDRs and/or VH CDRs comprising no more than 1, 2, 3, 4, 5, 6, 7, 8, 9, or 10 amino acid substitutions, deletions, or additions. In another specific embodiment, an antibody described herein, which immunospecifically binds to a KIT polypeptide (e.g., a D4/D5 region of KIT, for example human KIT), comprises one or more VL FRs and/or VH FRs comprising no more than 1, 2, 3, 4, 5, 6, 7, 8, 9, or 10 amino acid substitutions, deletions, or additions.

In a specific embodiment, an antibody described herein, which immunospecifically binds to a KIT polypeptide (e.g., a D4/D5 region of KIT, for example human KIT), comprises no more than 1, 2, 3, 4, 5, 6, 7, 8, 9, 10, 11, 12, 13, 14, 15, 16, 17, 18, 19, 20, 25, 30, 35, 40, 45, or 50 conservative amino acid substitutions, e.g., at one or more predicted non-essential amino acid residues. In another specific embodiment, an antibody described herein, which immunospecifically binds to a KIT polypeptide (e.g., a D4/D5 region of KIT, for example human KIT), comprises a light chain or a heavy chain comprising no more than 1, 2, 3, 4, 5, 6, 7, 8, 9, 10, 11, 12, 13, 14, 15, 16, 17, 18, 19, 20, 25, or 30 conservative amino acid substitutions, e.g., at one or more predicted non-essential amino acid residues. In another specific embodiment, an antibody described herein, which immunospecifically binds to a KIT polypeptide (e.g., a D4/D5 region of KIT, for example human KIT), comprises a VL chain region and/or a VH chain region comprising no more than 1, 2, 3, 4, 5, 6, 7, 8, 9, 10, 11, 12, 13, 14, 15, 16, 17, 18, 19, 20, 25, or conservative amino acid substitutions, e.g., at one or more predicted non-essential amino acid residues. In yet another specific embodiment, an antibody described herein, which immunospecifically binds to a KIT polypeptide (e.g., a D4/D5 region of KIT, for example human KIT), comprises a VL CDR and/or VH CDR comprising no more than 1, 2, 3, 4, 5, or 6 conservative amino acid substitutions, e.g., at one or more predicted non-essential amino acid residues. In another specific embodiment, an antibody described herein, which immunospecifically binds to a KIT polypeptide (e.g., a D4/D5 region of KIT, for example human KIT), comprises a VL FR and/or VH FR comprising no more than 1, 2, 3, 4, 5, 6, 7, 8, 9, or 10 conservative amino acid substitutions, e.g., at one or more predicted non-essential amino acid residues. Predicted non-essential amino acid residues of an antibody can readily be determined using methods known to one of skill in the art. For example, two-dimensional modeling or three-dimensional modeling of the structure of the antibody or its antigen-binding regions (for example, a VL domain (including, e.g., VL CDRs and VL FRs) and/or VH domain (including, e.g., VL CDRs and VL FRs)) can identify amino acid residues that are involved in antigen binding and/or amino acid residues that are important for the structure or conformation of the antibody. In specific embodiments, a two-dimensional model or three-dimensional model shows that a predicted non-essential amino acid residue is not involved in antigen-binding (see, e.g., Lo and Chen, Methods in Molecular Biol., 2004, 248:3-9; and Andrianov et al., J. Biomol. Struct. Dyn., 2009, 27(2):179-93). Structural studies can also be carried out by other methods known to one of skill in the art, for example x-ray crystallography. Another examples include mutagenesis screening assays, where amino acid residue mutations are introduced into antibodies (e.g., into the variable domains of the antibodies), and binding assays are carried out to determine which amino acid residue mutations disrupt antigen binding. The mutations can be introduced randomly or in a targeted manner. In specific embodiments, mutating a predicted non-essential amino acid residue does not affect antigen binding affinity, or negligibly (e.g., less than 10%, less than 5%, less than 3%, less than 2%, or less than 1%) affects antigen binding affinity, as determined for example by ELISA or Biacore™ assay.

In specific embodiments, one or more residues of a VH chain region, e.g., VH CDR or VH FR, or a VL chain region, e.g., VL CDR or VL FR, have been modified by affinity maturation. Certain affinity matured antibodies can comprise 1, 2, 3, 4, 5, 6, 7, 8, 9, or 10 amino acid substitutions in a VH CDR or VH FR sequence or VL CDR or VL FR sequence so that the affinity matured anti-KIT antibody has a higher affinity for the KIT antigen relative to the parent anti-KIT antibody which does not possess those alterations, as assessed by methods described herein and/or a method known to one of skill in the art (e.g., Biacore™ assay, assay using KinExA 3000 instrument, or ELISA). An affinity matured antibody can comprise 1, 2, 3, 4, 5, 6, 7, 8, 9, or 10 amino acid substitutions in one or more VH CDR sequences or one or more VL CDR sequences so that the affinity matured anti-KIT antibody has a higher affinity for the KIT antigen relative to the parent anti-KIT antibody which does not possess those alterations, as assessed by methods described herein and/or a method known to one of skill in the art (e.g., Biacore™ assay, assay using KinExA 3000 instrument or ELISA). In specific embodiments, an affinity mature anti-KIT antibody has an affinity for a KIT antigen (for example, a D4/D5 region of KIT, e.g., human KIT, for example SEQ ID NO: 171) that is at least 10%, 20%, 30%, 40%, 50%, 60%, 70%, 80%, 90%, 95%, or 99% higher than the affinity of the parent anti-KIT antibody which does not contain affinity matured alterations, as assessed by methods described herein and/or a method known to one of skill in the art (e.g., Biacore™ assay, assay using KinExA 3000 instrument, or ELISA). In specific embodiments, an affinity mature anti-KIT antibody has an affinity for a KIT antigen (for example, a D4/D5 region of KIT, e.g., human KIT, for example SEQ ID NO: 171) that is at least 1 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, or 100 fold higher than the affinity of the parent anti-KIT antibody which does not contain affinity matured alterations, as assessed by methods described herein and/or a method known to one of skill in the art (e.g., Biacore™ assay, assay using KinExA 3000 instrument, or ELISA).

In certain embodiments, an antibody described herein comprises a VL chain region having at least 80%, at least 82%, at least 85%, at least 87%, at least 88%, at least 89%, at least 90%, at least 92%, at least 95%, or at least 98% sequence identity to the amino acid sequence of SEQ ID NO: 176, 350, or 963 (Ab1), SEQ ID NO: 177 (Ab2), SEQ ID NO: 178 (Ab3), SEQ ID NO: 179 (Ab4), SEQ ID NO: 180 (Ab5), SEQ ID NO: 181 (Ab6), SEQ ID NO: 182 (Ab7), SEQ ID NO: 183 (Ab8), SEQ ID NO: 184 (Ab9), SEQ ID NO: 185 (Ab10), SEQ ID NO: 186 (Ab11), SEQ ID NO: 187 (Ab12), or SEQ ID NO: 328 or 964 (Ab21), wherein the antibody immunospecifically binds to a KIT polypeptide, e.g., a human KIT polypeptide, for example, a D4/D5 region of KIT (e.g., human KIT, for example SEQ ID NO: 171). In specific embodiments, the framework regions of such VL chain region comprises one or more amino acid substitutions (e.g., conservative amino acid substitutions). In specific embodiments, the sequences of the CDRs of the VL chain region do not contain any amino acid substitutions (e.g., conservative amino acid substitutions). For example, a VL chain region having at least 80%, at least 82%, at least 85%, at least 87%, at least 88%, at least 89%, at least 90%, at least 92%, at least 95%, or at least 98% sequence identity to the amino acid sequence of SEQ ID NO: 176 or 350 (Ab1), SEQ ID NO: 177 (Ab2), SEQ ID NO: 178 (Ab3), SEQ ID NO: 179 (Ab4), SEQ ID NO: 180 (Ab5), SEQ ID NO: 181 (Ab6), SEQ ID NO: 182 (Ab7), SEQ ID NO: 183 (Ab8), SEQ ID NO: 184 (Ab9), SEQ ID NO: 185 (Ab10), SEQ ID NO: 186 (Ab11), SEQ ID NO: 187 (Ab12), or SEQ ID NO: 328 (Ab21), comprises CDRs that are identical to those of SEQ ID NO: 176 or 350 (Ab1), SEQ ID NO: 177 (Ab2), SEQ ID NO: 178 (Ab3), SEQ ID NO: 179 (Ab4), SEQ ID NO: 180 (Ab5), SEQ ID NO: 181 (Ab6), SEQ ID NO: 182 (Ab7), SEQ ID NO: 183 (Ab8), SEQ ID NO: 184 (Ab9), SEQ ID NO: 185 (Ab10), SEQ ID NO: 186 (Ab11), SEQ ID NO: 187 (Ab12), or SEQ ID NO: 328 (Ab21), respectively.

In certain embodiments, an antibody described herein comprises a VL chain region having at least 80%, at least 82%, at least 85%, at least 87%, at least 88%, at least 89%, at least 90%, at least 92%, at least 95%, or at least 98% sequence identity to the amino acid sequence of SEQ ID NO: 176 or 350 (Ab1), wherein the antibody immunospecifically binds to a KIT polypeptide, e.g., a human KIT polypeptide, for example, a D4/D5 region of KIT (e.g., human KIT, for example SEQ ID NO: 171). In specific embodiments, the framework regions of such VL chain region comprises one or more amino acid substitutions (e.g., conservative amino acid substitutions). In specific embodiments, the sequences of the CDRs of such VL chain region do not contain any amino acid substitutions (e.g., conservative amino acid substitutions). For example, a VL chain region having at least 80%, at least 82%, at least 85%, at least 87%, at least 88%, at least 89%, at least 90%, at least 92%, at least 95%, or at least 98% sequence identity to the amino acid sequence of SEQ ID NO: 176, 350, or 963 (Ab1), comprises CDRs that are identical to those of SEQ ID NO: 176, 350, or 963 (Ab1), respectively.

In certain embodiments, an antibody described herein comprises a VL chain region having at least 80%, at least 82%, at least 85%, at least 87%, at least 88%, at least 89%, at least 90%, at least 92%, at least 95%, or at least 98% sequence identity to the amino acid sequence of SEQ ID NO: 328 (Ab21), wherein the antibody immunospecifically binds to a KIT polypeptide, e.g., a human KIT polypeptide, for example, a D4/D5 region of KIT (e.g., human KIT, for example SEQ ID NO: 171). In specific embodiments, the framework regions of such VL chain region comprises one or more amino acid substitutions (e.g., conservative amino acid substitutions). In specific embodiments, the sequences of the CDRs do not contain any amino acid substitutions (e.g., conservative amino acid substitutions). For example, a VL chain region having at least 80%, at least 82%, at least 85%, at least 87%, at least 88%, at least 89%, at least 90%, at least 92%, at least 95%, or at least 98% sequence identity to the amino acid sequence of SEQ ID NO: 328 or 964 (Ab21), comprises CDRs that are identical to those of SEQ ID NO: 328 or 964 (Ab21).

In certain embodiments, an antibody described herein comprises a VL chain region having at least 80%, at least 82%, at least 85%, at least 87%, at least 88%, at least 89%, at least 90%, at least 92%, at least 95%, or at least 98% sequence identity to the amino acid sequence of any one of SEQ ID NOs: 312-319. In specific embodiments, the framework regions of such VL chain region comprises one or more amino acid substitutions. In specific embodiments, the sequences of the CDRs do not contain any amino acid substitutions. For example, a VL chain region having at least 80%, at least 82%, at least 85%, at least 87%, at least 88%, at least 89%, at least 90%, at least 92%, at least 95%, or at least 98% sequence identity to the amino acid sequence of any one of SEQ ID NOs: 312-319, comprises CDRs that are identical to those of any one of SEQ ID NOs: 312-319, respectively.

In a specific embodiment, a VL chain region having at least 80%, at least 82%, at least 85%, at least 87%, at least 88%, at least 89%, at least 90%, at least 92%, at least 95%, or at least 98% sequence identity to the amino acid sequence of any one of SEQ ID NOs: 312-319, comprises one or more conservative amino acid substitution. In specific embodiments, the one or more conservative amino acid substitutions are in the framework regions of the VL chain region. In specific embodiments, the sequences of the CDRs of the VL chain region do not contain the one or more conservative amino acid substitutions. For example, a VL chain region having at least 80%, at least 82%, at least 85%, at least 87%, at least 88%, at least 89%, at least 90%, at least 92%, at least 95%, or at least 98% sequence identity to the amino acid sequence of any one of SEQ ID NOs: 312-319, comprises one or more conservative amino acid substitutions, and comprises CDRs that are identical to those of any one of SEQ ID NOs: 312-319, respectively.

In certain embodiments, an antibody described herein comprises a VL chain region comprising VL framework regions having at least 80%, at least 82%, at least 85%, at least 87%, at least 88%, at least 89%, at least 90%, at least 92%, at least 95%, or at least 98% sequence identity to the amino acid sequence of the framework regions of SEQ ID NO: 176 or 350 (Ab1), SEQ ID NO: 177 (Ab2), SEQ ID NO: 178 (Ab3), SEQ ID NO: 179 (Ab4), SEQ ID NO: 180 (Ab5), SEQ ID NO: 181 (Ab6), SEQ ID NO: 182 (Ab7), SEQ ID NO: 183 (Ab8), SEQ ID NO: 184 (Ab9), SEQ ID NO: 185 (Ab10), SEQ ID NO: 186 (Ab11), SEQ ID NO: 187 (Ab12), or SEQ ID NO: 328 (Ab21), wherein the antibody immunospecifically binds to a KIT polypeptide, e.g., a human KIT polypeptide, for example, a D4/D5 region of KIT (e.g., human KIT, for example SEQ ID NO: 171). In specific embodiments, such VL framework region comprises one or more conservative amino acid substitutions. In certain embodiments, an antibody described herein comprises a VL chain region comprising VL framework regions having at least 80%, at least 82%, at least 85%, at least 87%, at least 88%, at least 89%, at least 90%, at least 92%, at least 95%, or at least 98% sequence identity to the amino acid sequence of the framework regions of SEQ ID NO: 176 or 350 or 963 (Ab1), wherein the antibody immunospecifically binds to a KIT polypeptide, e.g., a human KIT polypeptide, for example, a D4/D5 region of KIT (e.g., human KIT, for example SEQ ID NO: 171). In certain embodiments, an antibody described herein comprises a VL chain region comprising VL framework regions having at least 80%, at least 82%, at least 85%, at least 87%, at least 88%, at least 89%, at least 90%, at least 92%, at least 95%, or at least 98% sequence identity to the amino acid sequence of the framework regions of SEQ ID NO: 328 or 964 (Ab21), wherein the antibody immunospecifically binds to a KIT polypeptide, e.g., a human KIT polypeptide, for example, a D4/D5 region of KIT (e.g., human KIT, for example SEQ ID NO: 171). In specific embodiments, such VL framework region comprises one or more conservative amino acid substitutions relative to the framework regions of Ab1 or Ab21.

In certain embodiments, an antibody described herein comprises a VH chain region having at least 80%, at least 82%, at least 83%, at least 84%, at least 85%, at least 88%, at least 90%, at least 93%, at least 95%, or at least 98% sequence identity to the amino acid sequence of SEQ ID NO: 188 (Ab1), SEQ ID NO: 189 (Ab2), SEQ ID NO: 190 (Ab3), SEQ ID NO: 191 (Ab4), SEQ ID NO: 192 (Ab5), SEQ ID NO: 193 (Ab6), SEQ ID NO: 194 (Ab7), SEQ ID NO: 195 (Ab8), SEQ ID NO: 196 (Ab9), SEQ ID NO: 197 (Ab10), SEQ ID NO: 198 (Ab11), SEQ ID NO: 199 (Ab12), or SEQ ID NO: 329 (Ab21), wherein the antibody immunospecifically binds to a KIT polypeptide, e.g., a human KIT polypeptide, for example, a D4/D5 region of KIT (e.g., human KIT, for example SEQ ID NO: 171). In specific embodiments, such VH chain region comprises one or more amino acid substitutions (e.g., conservative amino acid substitutions). In specific embodiments, the framework regions of such VH chain region comprises one or more amino acid substitutions (e.g., conservative amino acid substitutions). In specific embodiments, the sequences of the CDRs of the VH chain region do not contain any amino acid substitutions (e.g., conservative amino acid substitutions). For example, a VH chain region having at least 80%, at least 82%, at least 83%, at least 84%, at least 85%, at least 88%, at least 90%, at least 93%, at least 95%, or at least 98% sequence identity to the amino acid sequence of any one of SEQ ID NO: 188 (Ab1), SEQ ID NO: 189 (Ab2), SEQ ID NO: 190 (Ab3), SEQ ID NO: 191 (Ab4), SEQ ID NO: 192 (Ab5), SEQ ID NO: 193 (Ab6), SEQ ID NO: 194 (Ab7), SEQ ID NO: 195 (Ab8), SEQ ID NO: 196 (Ab9), SEQ ID NO: 197 (Ab10), SEQ ID NO: 198 (Ab11), SEQ ID NO: 199 (Ab12), or SEQ ID NO: 329 (Ab21), comprises CDRs that are identical to those of any one of SEQ ID NO: 188 (Ab1), SEQ ID NO: 189 (Ab2), SEQ ID NO: 190 (Ab3), SEQ ID NO: 191 (Ab4), SEQ ID NO: 192 (Ab5), SEQ ID NO: 193 (Ab6), SEQ ID NO: 194 (Ab7), SEQ ID NO: 195 (Ab8), SEQ ID NO: 196 (Ab9), SEQ ID NO: 197 (Ab10), SEQ ID NO: 198 (Ab11), SEQ ID NO: 199 (Ab12), or SEQ ID NO: 329 (Ab21), respectively.

In certain embodiments, an antibody described herein comprises a VH chain region having at least 80%, at least 82%, at least 83%, at least 84%, at least 85%, at least 90%, at least 93%, at least 95%, or at least 98% sequence identity to the amino acid sequence of SEQ ID NO: 188 (Ab1), wherein the antibody immunospecifically binds to a KIT polypeptide, e.g., a human KIT polypeptide, for example, a D4/D5 region of KIT (e.g., human KIT, for example SEQ ID NO: 171). In a specific embodiment, an antibody described herein comprises a VH chain region having at least 80% or at least 90% sequence identity to the amino acid sequence of SEQ ID NO: 188. In specific embodiments, such VH chain region comprises one or more conservative amino acid substitutions relative to the amino acid sequence of SEQ ID NO: 188 (Ab1). In specific embodiments, the framework regions of such VH chain region comprises one or more amino acid substitutions (e.g., conservative amino acid substitutions). In specific embodiments, the sequences of the CDRs do not contain any amino acid substitutions (e.g., conservative amino acid substitutions). For example, a VH chain region having at least 80%, at least 82%, at least 83%, at least 84%, at least 85%, at least 90%, at least 95%, or at least 98% sequence identity to the amino acid sequence of SEQ ID NO: 188 (Ab1), comprises CDRs that are identical to those of SEQ ID NO: 188 (Ab1).

In certain embodiments, an antibody described herein comprises a VH chain region having at least 80%, at least 82%, at least 83%, at least 84%, at least 85%, at least 90%, at least 93%, at least 95%, or at least 98% sequence identity to the amino acid sequence of SEQ ID NO: 329 (Ab21), wherein the antibody immunospecifically binds to a KIT polypeptide, e.g., a human KIT polypeptide, for example, a D4/D5 region of KIT (e.g., human KIT, for example SEQ ID NO: 171). In specific embodiments, such VH chain region comprises one or more amino acid substitutions (e.g., conservative amino acid substitutions) relative to the amino acid sequence of SEQ ID NO: 329 (Ab21). In specific embodiments, the sequences of the CDRs of such VH chain region do not contain any amino acid substitutions (e.g., conservative amino acid substitutions). For example, a VH chain region having at least 80%, at least 82%, at least 83%, at least 84%, at least 85%, at least 90%, at least 93%, at least 95%, or at least 98% sequence identity to the amino acid sequence of SEQ ID NO: 329 (Ab21), comprises CDRs that are identical to those of SEQ ID NO: 329 (Ab21).

In certain embodiments, an antibody described herein comprises a VH chain region having at least 80%, at least 82%, at least 83%, at least 84%, at least 85%, at least 90%, at least 93%, at least 95%, or at least 98% sequence identity to the amino acid sequence of any one of SEQ ID NOs: 320-327, and optionally having one or more conservative amino acid substitutions, wherein the antibody immunospecifically binds to a KIT polypeptide, e.g., a human KIT polypeptide, for example, a D4/D5 region of KIT (e.g., human KIT, for example SEQ ID NO: 171). In specific embodiments, the framework regions of such VH chain region comprises one or more amino acid substitutions (e.g., conservative amino acid substitutions). In specific embodiments, the sequences of the CDRs do not contain any amino acid substitutions (e.g., conservative amino acid substitutions). For example, a VH chain region having at least 80%, at least 82%, at least 83%, at least 84%, at least 85%, at least 90%, at least 93%, at least 95%, or at least 98% sequence identity to the amino acid sequence of any one of SEQ ID NOs: 320-327, comprises CDRs that are identical to those of any one of SEQ ID NOs: 320-327, respectively.

In certain embodiments, an antibody described herein comprises a VH chain region comprising VH framework regions having at least 80%, at least 82%, at least 83%, at least 84%, at least 85%, at least 90%, at least 93%, at least 95%, or at least 98% sequence identity to the amino acid sequence of the framework regions of SEQ ID NO: 188 (Ab1), SEQ ID NO: 189 (Ab2), SEQ ID NO: 190 (Ab3), SEQ ID NO: 191 (Ab4), SEQ ID NO: 192 (Ab5), SEQ ID NO: 193 (Ab6), SEQ ID NO: 194 (Ab7), SEQ ID NO: 195 (Ab8), SEQ ID NO: 196 (Ab9), SEQ ID NO: 197 (Ab10), SEQ ID NO: 198 (Ab11), SEQ ID NO: 199 (Ab12), or SEQ ID NO: 329 (Ab21), wherein the antibody immunospecifically binds to a KIT polypeptide, e.g., a human KIT polypeptide, for example, a D4/D5 region of KIT (e.g., human KIT, for example SEQ ID NO: 171). In certain embodiments, such VH framework regions comprises one or more conservative amino acid substitutions. In certain embodiments, an antibody described herein comprises a VH chain region comprising VH framework regions having at least 80%, at least 82%, at least 83%, at least 84%, at least 85%, at least 90%, at least 93%, at least 95%, or at least 98% sequence identity to the amino acid sequence of the framework regions of SEQ ID NO: 188 (Ab1), wherein the antibody immunospecifically binds to a KIT polypeptide, e.g., a human KIT polypeptide, for example, a D4/D5 region of KIT (e.g., human KIT, for example SEQ ID NO: 171). In certain embodiments, such VH framework regions comprises one or more conservative amino acid substitutions relative to the amino acid sequence of the framework regions of SEQ ID NO: 188(Ab1). In certain embodiments, an antibody described herein comprises a VH chain region comprising VH framework regions having at least 80%, at least 85%, at least 90%, at least 93%, at least 95%, or at least 98% sequence identity to the amino acid sequence of the framework regions of SEQ ID NO: 329 (Ab21), wherein the antibody immunospecifically binds to a KIT polypeptide, e.g., a human KIT polypeptide, for example, a D4/D5 region of KIT (e.g., human KIT, for example SEQ ID NO: 171). In certain embodiments, such VH framework regions comprises one or more conservative amino acid substitutions relative to the framework regions of SEQ ID NO: 329 (Ab21).

In certain embodiments, an antibody described herein comprises (i) a VL chain region having at least 80%, at least 82%, at least 83%, at least 84%, at least 85%, at least 90%, at least 95%, or at least 98% sequence identity to the amino acid sequence of SEQ ID NO: 176 or 350 or 963 (VL domain of Ab1), and (ii) a VH chain region having at least 80%, at least 82%, at least 83%, at least 84%, at least 85%, at least 90%, at least 95%, or at least 98% sequence identity to the amino acid sequence of SEQ ID NO: 188 (VH domain of Ab1), wherein the antibody immunospecifically binds to a KIT polypeptide, e.g., a human KIT polypeptide, for example, a D4/D5 region of KIT (e.g., human KIT, for example SEQ ID NO: 171). In certain embodiments, such VL chain region and/or VH chain region comprises one or more conservative amino acid substitutions. In a further specific embodiments, such VL chain region and/or VH chain region does not comprise amino acid substitutions in its CDRs. In a further specific embodiments, such VL chain region and/or VH chain region comprises one, two, or three conservative amino acid substitutions in its CDRs while immunospecific binding to KIT (e.g., the D4/D5 region of human KIT) is maintained.

In certain embodiments, an antibody described herein comprises (i) a VL chain region having at least 80%, at least 82%, at least 83%, at least 84%, at least 85%, at least 88%, at least 90%, at least 93%, at least 95%, or at least 98% sequence identity to the amino acid sequence of SEQ ID NO: 177 (VL domain of Ab2), and (ii) a VH chain region having at least 80%, at least 85%, at least 90%, at least 95%, or at least 98% sequence identity to the amino acid sequence of SEQ ID NO: 189 (VH domain of Ab2), wherein the antibody immunospecifically binds to a KIT polypeptide, e.g., a human KIT polypeptide, for example, a D4/D5 region of KIT (e.g., human KIT, for example SEQ ID NO: 171). In certain embodiments, such VL chain region and/or VH chain region comprises one or more conservative amino acid substitutions.

In certain embodiments, an antibody described herein comprises (i) a VL chain region having at least 80%, at least 82%, at least 83%, at least 84%, at least 85%, at least 88%, at least 90%, at least 93%, at least 95%, or at least 98% sequence identity to the amino acid sequence of SEQ ID NO: 178 (VL domain of Ab3), and (ii) a VH chain region having at least 80%, at least 82%, at least 83%, at least 84%, at least 85%, at least 88%, at least 90%, at least 93%, at least 95%, or at least 98% sequence identity to the amino acid sequence of SEQ ID NO: 190 (VH domain of Ab3), wherein the antibody immunospecifically binds to a KIT polypeptide, e.g., a human KIT polypeptide, for example, a D4/D5 region of KIT (e.g., human KIT, for example SEQ ID NO: 171). In certain embodiments, such VL chain region and/or VH chain region comprises one or more conservative amino acid substitutions.

In certain embodiments, an antibody described herein comprises (i) a VL chain region having at least 80%, at least 85%, at least 90%, at least 95%, or at least 98% sequence identity to the amino acid sequence of SEQ ID NO: 179 (VL domain of Ab4), and (ii) a VH chain region having at least 80%, at least 85%, at least 90%, at least 95%, or at least 98% sequence identity to the amino acid sequence of SEQ ID NO: 191 (VH domain of Ab4), wherein the antibody immunospecifically binds to a KIT polypeptide, e.g., a human KIT polypeptide, for example, a D4/D5 region of KIT (e.g., human KIT, for example SEQ ID NO: 171). In certain embodiments, such VL chain region and/or VH chain region comprises one or more conservative amino acid substitutions.

In certain embodiments, an antibody described herein comprises (i) a VL chain region having at least 80%, at least 85%, at least 90%, at least 95%, or at least 98% sequence identity to the amino acid sequence of SEQ ID NO: 180 (VL domain of Ab5), and (ii) a VH chain region having at least 80%, at least 85%, at least 90%, at least 95%, or at least 98% sequence identity to the amino acid sequence of SEQ ID NO: 192 (VH domain of Ab5), wherein the antibody immunospecifically binds to a KIT polypeptide, e.g., a human KIT polypeptide, for example, a D4/D5 region of KIT (e.g., human KIT, for example SEQ ID NO: 171). In certain embodiments, such VL chain region and/or VH chain region comprises one or more conservative amino acid substitutions.

In certain embodiments, an antibody described herein comprises (i) a VL chain region having at least 80%, at least 85%, at least 90%, at least 95%, or at least 98% sequence identity to the amino acid sequence of SEQ ID NO: 181 (VL domain of Ab6), and (ii) a VH chain region having at least 80%, at least 85%, at least 90%, at least 95%, or at least 98% sequence identity to the amino acid sequence of SEQ ID NO: 193 (VH domain of Ab6), wherein the antibody immunospecifically binds to a KIT polypeptide, e.g., a human KIT polypeptide, for example, a D4/D5 region of KIT (e.g., human KIT, for example SEQ ID NO: 171). In certain embodiments, such VL chain region and/or VH chain region comprises one or more conservative amino acid substitutions.

In certain embodiments, an antibody described herein comprises (i) a VL chain region having at least 80%, at least 85%, at least 90%, at least 95%, or at least 98% sequence identity to the amino acid sequence of SEQ ID NO: 182 (VL domain of Ab7), and (ii) a VH chain region having at least 80%, at least 85%, at least 90%, at least 95%, or at least 98% sequence identity to the amino acid sequence of SEQ ID NO: 194 (VH domain of Ab7), wherein the antibody immunospecifically binds to a KIT polypeptide, e.g., a human KIT polypeptide, for example, a D4/D5 region of KIT (e.g., human KIT, for example SEQ ID NO: 171). In certain embodiments, such VL chain region and/or VH chain region comprises one or more conservative amino acid substitutions.

In certain embodiments, an antibody described herein comprises (i) a VL chain region having at least 80%, at least 85%, at least 90%, at least 95%, or at least 98% sequence identity to the amino acid sequence of SEQ ID NO: 183 (VL domain of Ab8), and (ii) a VH chain region having at least 80%, at least 85%, at least 90%, at least 95%, or at least 98% sequence identity to the amino acid sequence of SEQ ID NO: 195 (VH domain of Ab8), wherein the antibody immunospecifically binds to a KIT polypeptide, e.g., a human KIT polypeptide, for example, a D4/D5 region of KIT (e.g., human KIT, for example SEQ ID NO: 171). In certain embodiments, such VL chain region and/or VH chain region comprises one or more conservative amino acid substitutions.

In certain embodiments, an antibody described herein comprises (i) a VL chain region having at least 80%, at least 85%, at least 90%, at least 95%, or at least 98% sequence identity to the amino acid sequence of SEQ ID NO: 184 (VL domain of Ab9), and (ii) a VH chain region having at least 80%, at least 85%, at least 90%, at least 95%, or at least 98% sequence identity to the amino acid sequence of SEQ ID NO: 196 (VH domain of Ab9), wherein the antibody immunospecifically binds to a KIT polypeptide, e.g., a human KIT polypeptide, for example, a D4/D5 region of KIT (e.g., human KIT, for example SEQ ID NO: 171). In certain embodiments, such VL chain region and/or VH chain region comprises one or more conservative amino acid substitutions.

In certain embodiments, an antibody described herein comprises (i) a VL chain region having at least 80%, at least 85%, at least 90%, at least 95%, or at least 98% sequence identity to the amino acid sequence of SEQ ID NO: 185 (VL domain of Ab10), and (ii) a VH chain region having at least 80%, at least 85%, at least 90%, at least 95%, or at least 98% sequence identity to the amino acid sequence of SEQ ID NO: 197 (VH domain of Ab10), wherein the antibody immunospecifically binds to a KIT polypeptide, e.g., a human KIT polypeptide, for example, a D4/D5 region of KIT (e.g., human KIT, for example SEQ ID NO: 171). In certain embodiments, such VL chain region and/or VH chain region comprises one or more conservative amino acid substitutions.

In certain embodiments, an antibody described herein comprises (i) a VL chain region having at least 80%, at least 85%, at least 90%, at least 95%, or at least 98% sequence identity to the amino acid sequence of SEQ ID NO: 186 (VL domain of Ab11), and (ii) a VH chain region having at least 80%, at least 85%, at least 90%, at least 95%, or at least 98% sequence identity to the amino acid sequence of SEQ ID NO: 198 (VH domain of Ab11), wherein the antibody immunospecifically binds to a KIT polypeptide, e.g., a human KIT polypeptide, for example, a D4/D5 region of KIT (e.g., human KIT, for example SEQ ID NO: 171). In certain embodiments, such VL chain region and/or VH chain region comprises one or more conservative amino acid substitutions.

In certain embodiments, an antibody described herein comprises (i) a VL chain region having at least 80%, at least 85%, at least 90%, at least 95%, or at least 98% sequence identity to the amino acid sequence of SEQ ID NO: 187 (VL domain of Ab12), and (ii) a VH chain region having at least 80%, at least 85%, at least 90%, at least 95%, or at least 98% sequence identity to the amino acid sequence of SEQ ID NO: 199 (VH domain of Ab12), wherein the antibody immunospecifically binds to a KIT polypeptide, e.g., a human KIT polypeptide, for example, a D4/D5 region of KIT (e.g., human KIT, for example SEQ ID NO: 171). In certain embodiments, such VL chain region and/or VH chain region comprises one or more conservative amino acid substitutions.

In certain embodiments, an antibody described herein comprises (i) a VL chain region having at least 80%, at least 82%, at least 83%, at least 84%, at least 85%, at least 88%, at least 90%, at least 93%, at least 95%, or at least 98% sequence identity to the amino acid sequence of SEQ ID NO: 328 or 964 (VL domain of Ab21), and (ii) a VH chain region having at least 80%, at least 82%, at least 83%, at least 84%, at least 85%, at least 88%, at least 90%, at least 93%, at least 95%, or at least 98% sequence identity to the amino acid sequence of SEQ ID NO: 329 (VH domain of Ab21), wherein the antibody immunospecifically binds to a KIT polypeptide, e.g., a human KIT polypeptide, for example, a D4/D5 region of KIT (e.g., human KIT, for example SEQ ID NO: 171). In certain embodiments, such a VL chain region and/or a VH chain region comprises one or more conservative amino acid substitutions. In a further specific embodiment, such a VL chain region and/or a VH chain region do not comprise amino acid substitutions in its CDRs. In a further specific embodiments, such a VL chain region and/or a VH chain region comprise one, two, or three conservative amino acid substitutions in its CDRs while immunospecific binding to KIT (e.g., the D4/D5 region of human KIT) is maintained.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions X 100%). In one embodiment, the two sequences are the same length.

The determination of percent identity between two sequences (e.g., amino acid sequences or nucleic acid sequences) can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm nih.gov). Another preferred, non limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In particular embodiments, the glycosylation of antibodies described herein is modified. For example, an aglycoslated antibody can be made (i.e., the antibody lacks glycosylation) or an antibody comprising a mutation or substitution at one or more glycosylation sites to eliminate glycosylation at the one or more glycosylation sites can be be made. Glycosylation can be altered to, for example, increase the affinity of the antibody for a target antigen (e.g., human KIT, for example, a D4/D5 region of human KIT). Such carbohydrate modifications can be accomplished by, for example, altering one or more sites of glycosylation within the antibody sequence. For example, one or more amino acid substitutions can be made that result in elimination of one or more variable region (e.g., VL and/or VH CDRs or VL and/or VH FRs) glycosylation sites to thereby eliminate glycosylation at that site. Such aglycosylation can increase the affinity of the antibody for antigen (e.g., human KIT, for example, a D4/D5 region of human KIT). Such an approach is described in further detail in U.S. Pat. Nos. 5,714,350 and 6,350,861.

Glycosylation can occur via N-linked (or asparagine-linked) glycosylation or O-linked glycosylation. N-linked glycosylation involves carbohydrate modification at the side-chain NH₂ group of an asparagine amino acid in a polypeptide. O-linked glycosylation involves carbohydrate modification at the hydroxyl group on the side chain of a serine, threonine, or hydroxylysine amino acid.

In specific embodiments, an asparagine (N) residue within a VH or VL region of an antibody described herein is substituted with a serine (S) or another amino acid (e.g., alanine, glycine, glutamine, serine, threonine, tyrosine, cysteine). In other specific embodiments, an asparagine (N) residue within a VH CDR (e.g., VH CDR1, VH CDR2, and/or VH CDR3) and/or a VL CDR (e.g., VL CDR1, VL CDR2, and/or VL CDR3) of an antibody described herein is substituted with a serine (S) or another amino acid (e.g., alanine, glycine, glutamine, serine, threonine, tyrosine, cysteine). In other specific embodiments, an asparagine (N) residue within a VL CDR3 sequence (e.g., SEQ ID NO:26) of an antibody described herein is substituted with a serine (S) or another amino acid (e.g., alanine, glycine, glutamine, serine, threonine, tyrosine, cysteine). In other specific embodiments, an asparagine (N) residue within a VH CDR1 sequence of an antibody described herein is substituted with a serine (S) or another amino acid (e.g., alanine, glycine, glutamine, serine, threonine, tyrosine, cysteine). In other specific embodiments, an asparagine (N) residue within a VH FR (e.g., VH FR1, VH FR2, VH FR3 and/or VH FR4) and/or a VL FR (e.g., VL FR1, VL FR2, VL FR3, and/or VL FR4) of an antibody described herein is substituted with a serine (S) or another amino acid (e.g., alanine, glycine, glutamine, serine, threonine, tyrosine, cysteine).

In other specific embodiments, provided herein is an antibody, which immunospecifically binds to a KIT polypeptide (e.g., a human KIT polypeptide, for example, a D4/D5 region of KIT, e.g., human KIT, for example SEQ ID NO: 171), comprising (i) a VL chain region comprising a VL CDR1, VL CDR2, and VL CDR3 having the amino acid sequences of SEQ ID NO: 2 (VL CDR1 of Ab1), SEQ ID NO: 14 (VL CDR2 of Ab1), and SEQ ID NO: 26 (VL CDR3 of Ab1), respectively; and (ii) a VH chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively; wherein an an asparagine (N) residue within VL CDR3 (SEQ ID NO: 26) is substituted with a serine (S) or another amino acid (e.g., alanine, glycine, glutamine, serine, threonine, tyrosine, cysteine).

In other specific embodiments, provided herein is an antibody, which immunospecifically binds to a KIT polypeptide (e.g., a human KIT polypeptide, for example, a D4/D5 region of KIT, e.g., human KIT, for example SEQ ID NO: 171), comprising (i) a VL chain region comprising a VL CDR1, VL CDR2, and VL CDR3 having the amino acid sequences of SEQ ID NO: 200 (VL CDR1 of Ab13), SEQ ID NO: 208 (VL CDR2 of Ab13), and SEQ ID NO: 216 (VL CDR3 of Ab13), respectively; and (ii) a VH chain region comprising a VH CDR1, VH CDR2, and VH CDR3 having the amino acid sequences of SEQ ID NO: 256 (VH CDR1 of Ab13), SEQ ID NO: 264 (VH CDR2 of Ab13), and SEQ ID NO: 272 (VH CDR3 of Ab13), respectively; wherein an an asparagine (N) residue within VH CDR1 (SEQ ID NO: 256) is substituted with a serine (S) or another amino acid (e.g., alanine, glycine, glutamine, serine, threonine, tyrosine, cysteine).

In other specific embodiments, provided herein is an antibody, which immunospecifically binds to a KIT polypeptide (e.g., a human KIT polypeptide, for example, a D4/D5 region of KIT, e.g., human KIT, for example SEQ ID NO: 171), comprising (i) a VL chain region comprising a VL CDR1, VL CDR2, and VL CDR3 having the amino acid sequences of SEQ ID NO: 202 (VL CDR1 of Ab15), SEQ ID NO: 210 (VL CDR2 of Ab15), and SEQ ID NO: 218 (VL CDR3 of Ab15), respectively; and (ii) a VH chain region comprising a VH CDR1, VH CDR2, and VH CDR3 having the amino acid sequences of SEQ ID NO: 258 (VH CDR1 of Ab15), SEQ ID NO: 266 (VH CDR2 of Ab15), and SEQ ID NO: 274 (VH CDR3 of Ab15), respectively; wherein an an asparagine (N) residue within VH CDR1 (SEQ ID NO: 258) is substituted with a serine (S) or another amino acid (e.g., alanine, glycine, glutamine, serine, threonine, tyrosine, cysteine).

In other specific embodiments, provided herein is an antibody, which immunospecifically binds to a KIT polypeptide (e.g., a human KIT polypeptide, for example, a D4/D5 region of KIT, e.g., human KIT, for example SEQ ID NO: 171), comprising (i) a VL chain region comprising a VL CDR1, VL CDR2, and VL CDR3 having the amino acid sequences of SEQ ID NO: 204 (VL CDR1 of Ab17), SEQ ID NO: 212 (VL CDR2 of Ab17), and SEQ ID NO: 220 (VL CDR3 of Ab17), respectively; and (ii) a VH chain region comprising a VH CDR1, VH CDR2, and VH CDR3 having the amino acid sequences of SEQ ID NO: 260 (VH CDR1 of Ab17), SEQ ID NO: 268 (VH CDR2 of Ab17), and SEQ ID NO: 276 (VH CDR3 of Ab17), respectively; wherein an an asparagine (N) residue within VH CDR1 (SEQ ID NO: 260) is substituted with a serine (S) or another amino acid (e.g., alanine, glycine, glutamine, serine, threonine, tyrosine, cysteine).

In certain embodiments, aglycosylated antibodies can be produced in bacterial cells which lack the necessary glycosylation machinery. Cells with altered glycosylation machinery have been described in the art and can be used as host cells in which to express recombinant antibodies described herein to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 03/035835; WO 99/54342.

In certain embodiments, one or more modifications can be made to the Fc region of an antibody described here, generally, to alter one or more functional properties of the antibody, such as serum half-life, complement fixation, Fc receptor binding, and/or antigen-dependent cellular cytotoxicity. These modifications are known in the art, and are described in for example, International Patent Application Publication No. WO 2008/153926 A2.

Provided herein are antibodies that immunospecifically bind to KIT antigen and that can modulate KIT activity. In certain embodiments, an antibody provided herein immunospecifically binds to a KIT polypeptide, e.g., a human KIT polypeptide, and inhibits a KIT activity. KIT activity can relate to any activity of KIT known or described in the art, e.g., KIT receptor dimerization, KIT receptor phosphorylation (tyrosine phosphorylation), signaling downstream of the KIT receptor (e.g., AKT, MAPK/ERK, Ras, Stat1, Stat3, or Stat5 signaling), KIT ligand (e.g., SCF) induced transcriptional regulation (e.g., SCF-induced transcriptional activation of c-Myc), induction or enhancement of cell proliferation, or cell survival. KIT activity or KIT function are used interchangeably herein. In certain aspects, KIT activity is induced by KIT ligand (e.g., SCF) binding to KIT receptor. In particular aspects, KIT activity can be induced or enhanced by gain-of-function mutations which can result, for example, in dimerization and constitutive active KIT signaling (see, e.g., Mol et al., J. Biol. Chem., 2003, 278:31461-31464; Hirota et al., J. Pathology, 2001, 193:505-510). Such gain-of-function can allow for KIT receptor dimerization and KIT signaling to occur in the absence of KIT ligand (e.g., SCF) binding to KIT receptor. In certain embodiments, an increase in KIT activity or signaling can occur, in the absence of KIT ligand (e.g., SCF) binding KIT receptor, due to high (or overexpression) expression of KIT receptors. High or overexpression of KIT in a cell refers to an expression level which is at least about 10%, 20%, 30%, 40%, 50%, 60%, 70%, 80%, 90%, or 100% more than the expression level of a reference cell known to have normal KIT expression or KIT activity or more than the average expression level of KIT in a population of cells or samples known to have normal KIT expression or KIT activity. Expression levels of KIT can be assessed by methods described herein or known to one of skill in the art (e.g., Western blotting or immunohistochemistry). In particular embodiments, KIT activity that is higher than normal KIT activity can lead to cellular transformation, neoplasia, and tumorogenesis. In particular embodiments, KIT activity that is higher than normal KIT activity can lead to other KIT-mediated disorders or diseases.

In certain embodiments, an anti-KIT antibody described herein does not block or inhibit binding of KIT ligand (e.g., SCF) to KIT receptor. In certain embodiments, an anti-KIT antibody described herein only negligibly (e.g., less than about 2% or 3%) inhibits or reduces binding of KIT ligand (e.g., SCF) to KIT receptor. In certain embodiments, an anti-KIT antibody described herein does not induce or enhance dissociation of KIT ligand (e.g., SCF) from the KIT receptor. In certain embodiments, an anti-KIT antibody described herein only negligibly (e.g., less than about 2% or 3%) induces or enhances dissociation of KIT ligand (e.g., SCF) from the KIT receptor.

In specific embodiments, antibodies described herein specifically bind to an extracellular domain of KIT (e.g., D4/D5 region of KIT, for example human KIT) and block or inhibit binding of KIT ligand (e.g., SCF) to KIT by at least about 5%, 10%, 15%, 20%, 25%, 30%, 35%, 40%, 45%, 50%, 55%, 60%, 65%, 70%, 75%, 80%, 85%, 90%, 95%, or 98%, as assessed by methods described herein or known to one of skill in the art, e.g., ELISA assay, flow cytometry, or competition assay.

In certain aspects, inhibition by anti-KIT antibodies described herein (e.g., anti-KIT human monoclonal antibody) of KIT ligand (e.g., SCF) binding to KIT can be characterized by IC₅₀ values, which reflects the concentration of anti-KIT antibodies achieving 50% inhibition of binding of KIT ligand to KIT. Thus, in specific embodiments, an anti-KIT antibody described herein inhibits binding of KIT ligand to KIT with an IC₅₀ of at most about 10,000 nM, 1,000 nM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 5 nM, 1 nM, 0.75 nM, 0.5 nM, 0.1 nM, 0.05 nM, 0.01 nM, 0.005 nM, or 0.001 nM, as assessed by methods described herein and/or known to one of skill in the art, (e.g., ELISA assay or flow cytometry). In specific embodiments, an anti-KIT antibody described herein inhibits binding of KIT ligand to KIT with an IC₅₀ of at least about 10,000 nM, 1,000 nM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 5 nM, 1 nM, 0.75 nM, 0.5 nM, 0.1 nM, 0.05 nM, 0.01 nM, 0.005 nM, or 0.001 nM, as assessed by methods described herein and/or known to one of skill in the art, (e.g., ELISA assay or flow cytometry). In particular embodiments, an anti-KIT antibody described herein inhibits binding of KIT ligand to KIT with an IC₅₀ in the range of about 0.01 nM to 10,000 nM, 0.01 nM to 1,000 nM, 0.1 nM to 500 nM, 0.1 nM to 100 nM, or 0.1 nM to 50 nM, as assessed by methods described herein and/or known to one of skill in the art, (e.g., ELISA assay or flow cytometry).

In certain embodiments, an anti-KIT antibody described herein does not block or inhibit KIT receptor dimerization. In certain embodiments, an anti-KIT antibody described herein only negligibly (e.g., less than about 2% or 3%) inhibits or reduces KIT receptor dimerization. In certain embodiments, an anti-KIT antibody described herein does not induce or enhance KIT receptor dimer dissociation. In certain embodiments, an anti-KIT antibody described herein only negligibly (e.g., less than about 2% or 3%) induces or enhances KIT receptor dimer dissociation. In a particular embodiment, an anti-KIT antibody described herein can specifically bind to a KIT receptor dimer and do not block or inhibit KIT receptor dimerization. In a particular embodiment, an anti-KIT antibody described herein can specifically bind to a KIT receptor monomer and do not block or inhibit KIT receptor dimerization.

In certain aspects, as an inhibitor of KIT activity, an antibody described herein can block or inhibit dimerization of KIT. Generally, KIT receptor dimerization is induced when KIT ligand binds to KIT. Thus, in specific embodiments, antibodies described herein specifically bind to KIT and block or inhibit dimerization of KIT receptors by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art, e.g., immunoprecipitation assay, relative to dimerization of KIT receptors in the presence of KIT ligand stimulation without any antibody or with an unrelated antibody (e.g., an antibody that does not immunospecifically bind to KIT). Blocking or inhibition of dimerization of KIT receptors by antibodies described herein can be assessed in the presences of KIT ligand stimulation. For example, cells expressing KIT are contacted with KIT ligand in the presence or absence of anti-KIT antibodies described herein, and the level of KIT receptor dimerization is determined. In certain embodiments, KIT ligand induced KIT receptor dimerization in the absence of anti-KIT antibody is at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold higher than KIT receptor dimerization in the presence of anti-KIT antibody as assessed by methods described herein or known to one of skill in the art (e.g., immunoprecipitation assays). Tyrosine phosphorylation of one or more residues in the cytoplasmic domain of KIT can be an indicator of KIT receptor dimerization.

In certain embodiments, an antibody described herein can inhibit KIT activity by at least about 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein and/or known to one of skill in the art, relative to KIT activity in the presence of KIT ligand stimulation without any antibody or with an unrelated antibody (e.g., an antibody that does not immunospecifically bind to KIT). Non-limiting examples of KIT activity can include KIT receptor phosphorylation, KIT receptor signaling, KIT ligand (e.g., SCF) mediated cell proliferation, KIT ligand (e.g., SCF) mediated cell survival, and transcriptional activation of a KIT target gene (e.g., c-Myc).

As an inhibitor of KIT activity, an antibody described herein can block or inhibit phosphorylation of KIT, specifically tyrosine phosphorylation of one or more residues in the cytoplasmic domain of KIT. Generally, KIT receptor dimerization and phosphorylation is induced when KIT ligand binds to KIT. However, in certain aspects, KIT receptor dimerization and/or phosphorylation can occur independent of KIT ligand binding to KIT receptor. For example KIT receptor dimerization and/or phosphorylation can occur due to gain-of-function mutations or overexpression of KIT.

Non-limiting examples of tyrosine residues in the cytoplasmic domain of murine KIT that can be phosphorylated, e.g., upon ligand stimulation, include 544, 546, 552, 567, 569, 577, 608, 645, 671, 674, 702, 719, 728, 745, 772, 821, 844, 853, 868, 878, 898, and 934 (see Ueda et al., Blood, 2002, 99:3342-3349). The corresponding tyrosine residues in the cytoplasmic domain of human KIT can be readily determined. Non-limiting examples of tyrosine residues in the cytoplasmic domain of human KIT (e.g., GenBank Accession No. P10721) that can be phosphorylated, e.g., upon ligand stimulation, include residues 568, 570, 703, 721, 730, 747, 823, 900, and 936. In a specific embodiment, an anti-KIT antibody described herein can inhibits receptor phosphorylation at tyrosine residue 719 of murine KIT. In another specific embodiment, an anti-KIT antibody described herein can inhibits receptor phosphorylation at tyrosine residue 703 or 721 of human KIT.

Thus, in specific embodiments, antibodies described herein specifically bind to KIT and block or inhibit tyrosine phosphorylation in the cytoplasmic domain of KIT by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art, e.g., ELISA assay as described in section 6 or immunoblotting assay, relative to phosphorylation in the presence of KIT ligand stimulation without any antibody or with an unrelated antibody (e.g., an antibody that does not immunospecifically bind to KIT). In particular embodiments, antibodies described herein specifically bind to KIT and block or inhibit tyrosine phosphorylation in the cytoplasmic domain of KIT by at least about 25% as assessed by methods described herein or known to one of skill in the art, e.g., ELISA assay as described in section 6 or immunoblotting assay. In certain embodiments, antibodies described herein specifically bind to KIT and block or inhibit tyrosine phosphorylation in the cytoplasmic domain of KIT by at least about 25% to 80% as assessed by methods described herein or known to one of skill in the art, e.g., ELISA assay as described in section 6 or immunoblotting assay.

In specific embodiments, antibodies described herein specifically bind to KIT and reduce tyrosine phosphorylation in the cytoplasmic domain of KIT by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art, e.g., ELISA assay as described in section 6 or immunoblotting assay, relative to phosphorylation in the presence of KIT ligand stimulation without any antibody or with an unrelated antibody (e.g., an antibody that does not immunospecifically bind to KIT).

In specific embodiments, antibodies described herein specifically bind to KIT and block or inhibit phosphorylation of one or more tyrosine residues in the cytoplasmic domain of KIT by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art, e.g., immunoblotting assay, relative to phosphorylation in the presence of KIT ligand stimulation without any antibody or with an unrelated antibody (e.g., an antibody that does not immunospecifically bind to KIT). Blocking or inhibition of phosphorylation of one or more tyrosine residues of the cytoplasmic domain of KIT by antibodies described herein can be assessed upon KIT ligand stimulation. For example, cells expressing KIT are contacted with KIT ligand in the presence or absence of anti-KIT antibodies described herein, and the level of phosphorylation of one or more tyrosine residues in the cytoplasmic domain of KIT can be determined. In certain embodiments, KIT ligand induced phosphorylation of one or more tyrosine residues of the cytoplasmic domain of KIT the absence of anti-KIT antibody is at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold higher than KIT ligand induced phosphorylation of one or more tyrosine residues of the cytoplasmic domain of KIT in the presence of anti-KIT antibody, as assessed by methods described herein or known to one of skill in the art (e.g., immunoblotting assays), relative to phosphorylation in the presence of KIT ligand stimulation without any antibody or with an unrelated antibody (e.g., an antibody that does not immunospecifically bind to KIT).

In specific embodiments, antibodies described herein specifically bind to KIT and induce or enhance KIT receptor internalization by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art, relative to internalization in the presence of an unrelated antibody (e.g., an antibody that does not immunospecifically bind to KIT). In specific embodiments, antibodies described herein specifically bind to KIT and induce or enhance KIT receptor internalization by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art, relative to internalization in the presence of an unrelated antibody (e.g., an antibody that does not immunospecifically bind to KIT). Techniques for the quantitation or visualization of cell surface receptors are well known in the art and include a variety of fluorescent and radioactive techniques. For example, one method involves incubating the cells with a radiolabeled anti-receptor antibody. Alternatively, the natural ligand of the receptor can be conjugated to a fluorescent molecule or radioactive-label and incubated with the cells. Additional receptor internalization assays are well known in the art and are described in, for example, Jimenez et al., Biochemical Pharmacology, 1999, 57:1125-1131; Bernhagen et al., Nature Medicine, 2007, 13:587-596; and Conway et al., J. Cell Physiol., 2001, 189:341-55.
In specific embodiments, antibodies described herein specifically bind to KIT and induce or enhance KIT receptor turnover by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art (e.g., pulse-chase assay), relative to turnover in the presence of an unrelated antibody (e.g., an antibody that does not immunospecifically bind to KIT). In specific embodiments, antibodies described herein specifically bind to KIT and induce or enhance KIT receptor turnover by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art (e.g., pulse-chase assay), relative to turnover in the presence of an unrelated antibody (e.g., an antibody that does not immunospecifically bind to KIT). Methods for the determining receptor turnover are well known in the art. For example, cells expressing KIT can be pulse-labeled using ³⁵S-EXPRESS Protein Labeling mix (NEG772, NEN Life Science Products), washed and chased with unlabeled medium for a period of time before protein lysates from the labeled cells are immunoprecipitated using an anti-KIT antibody and resolved by SDS-PAGE and visualized (e.g., exposed to a Phospholmager screen (Molecular Dynamics), scanned using the Typhoon8600 scanner (Amersham), and analyzed using ImageQuant software (Molecular Dynamics)) (see, e.g., Chan et al., Development, 2004, 131:5551-5560).

In specific embodiments, antibodies described herein specifically bind to KIT and induce or enhance KIT receptor degradation by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art (e.g., pulse-chase assays), relative to degradation in the presence of an unrelated antibody (e.g., an antibody that does not immunospecifically bind to KIT). In specific embodiments, antibodies described herein specifically bind to KIT and induce or enhance KIT receptor degradation by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art (e.g., pulse-chase assays), relative to degradation in the presence of an unrelated antibody (e.g., an antibody that does not immunospecifically bind to KIT). Techniques for quantitating or monitoring ubiquitination and/or degradation (e.g., kinetics or rate of degradation) of cell surface receptors are well known in the art and involve a variety of fluorescent and radioactive techniques (see, e.g., International Patent Application Publication No. WO 2008/153926 A2). For example, pulse chase experiments or experiments using radiolabeled ligands such as ¹²⁵I-SCF can be carried out to quantitatively measure degradation of KIT.

Moreover, signaling events downstream of KIT receptor phosphorylation can serve as indicators of KIT activity. For example, KIT ligand (e.g., SCF) binding to its receptor KIT stimulates several distinct signaling pathways, including for example members of Src family kinases, phosphatidylinositol (PI) 3-kinases, and Ras mitogen-activated protein kinase (MAPK) (see Munugalavadla et al., Mol. Cell. Biol., 2005, 25:6747-6759). Phosphorylated tyrosines in the cytoplasmic domain of KIT can provide for binding sites for SH2 domain-containing proteins, which include, but are not limited to, proteins of the p21Ras-mitogen activated protein kinase (MAPK) pathway, the p85 subunit of PI 3-kinase, phospholipase C-gamma₁, the Grb2 adaptor protein, the Src family kinases (SFKs), Cb1, CRKL, p62Dok-1, SHP1, and SHP2 (see Ueda et al., Blood, 2002, 99:3342-3349).

Thus, in certain aspects, anti-KIT antibodies described herein which act as inhibitors of KIT activity can inhibit signaling of a member of the Src family kinases, PI 3-kinases, or Ras-MAPK. In particular embodiments, anti-KIT antibodies described herein which act as inhibitors of KIT activity can inhibit binding (or inhibit interaction), to the cytoplasmic domain of KIT, of one or more SH2 domain-containing proteins, such as proteins of the p21Ras-MAPK pathway, the p85 subunit of PI 3-kinase, phospholipase C-gammal, the Grb2 adaptor protein, a member of the SFK, Cb1, CRKL, p62Dok-1, SHP1, and SHP2. In certain embodiments, anti-KIT antibodies described herein which act as inhibitors of KIT activity can inhibit activation by KIT of one or more SH2 domain-containing proteins, such as proteins of the p21Ras-MAPK pathway, the p85 subunit of PI 3-kinase, phospholipase C-gammal, the Grb2 adaptor protein, a member of the SFK, Cb1, CRKL, p62Dok-1, SHP1, and SHP2.

In particular embodiments, anti-KIT antibodies described herein which act as inhibitors of KIT activity can inhibit downstream signaling such as phosphorylation of MAPK, phosphorylation of AKT, or phosphorylation of Stat1, Stat3, or Stat5. Thus, in certain embodiments, an anti-KIT antibody described herein can inhibit or reduce phosphorylation of MAPK (e.g., KIT ligand (e.g., SCF) induced phosphorylation of MAPK) by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art, e.g., Western blot or ELISA assay as described in section 6 or immunoblotting assay, relative to phosphorylation in the presence of KIT ligand stimulation without any antibody or with an unrelated antibody (e.g., an antibody that does not immunospecifically bind to KIT). In certain embodiments, an anti-KIT antibody described herein can inhibit or reduce phosphorylation of AKT (e.g., KIT ligand (e.g., SCF) induced phosphorylation of AKT) by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art, e.g., Western blot or ELISA assay as described in section 6 or immunoblotting assay, relative to phosphorylation in the presence of KIT ligand stimulation without any antibody or with an unrelated antibody (e.g., an antibody that does not immunospecifically bind to KIT). In particular embodiments, an anti-KIT antibody described herein can inhibit or reduce phosphorylation of Stat3 (e.g., KIT ligand (e.g., SCF) induced phosphorylation of Stat3) by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art, e.g., Western blot or ELISA assay as described in section 6 or immunoblotting assay, relative to phosphorylation in the presence of KIT ligand stimulation without any antibody or with an unrelated antibody (e.g., an antibody that does not immunospecifically bind to KIT). In particular embodiments, an anti-KIT antibody described herein can inhibit or reduce phosphorylation of Stat1 or Stat5 (e.g., KIT ligand (e.g., SCF) induced phosphorylation) by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art, e.g., Western blot or ELISA assay as described in section 6 or immunoblotting assay, relative to phosphorylation in the presence of KIT ligand stimulation without any antibody or with an unrelated antibody (e.g., an antibody that does not immunospecifically bind to KIT).

In certain aspects, an anti-KIT antibody described herein which can act as an inhibitor of KIT activity or activity can inhibit cellular proliferation of cells (e.g., TF-1 cells) that express KIT and that respond to KIT signaling (e.g., cells that proliferate in response to KIT ligand stimulation and KIT signaling). Cell proliferation assays are described in the art and can be readily carried out by one of skill in the art. For example, cell proliferation can be assayed by measuring Bromodeoxyuridine (BrdU) incorporation (see, e.g., Hoshino et al., 1986, Int. J. Cancer 38, 369; Campana et al., 1988, J. Immunol. Meth. 107:79) or (3H) thymidine incorporation (see, e.g., Blechman et al., Cell, 1995, 80:103-113; Chen, J., 1996, Oncogene 13:1395-403; Jeoung, J., 1995, J. Biol. Chem. 270:18367 73), by direct cell count at various time intervals (e.g., 12-hour or 24-hour intervals), or by detecting changes in transcription, translation or activity of known genes such as proto-oncogenes (e.g., fos, myc) or cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as ELISA, Western blotting or immunoprecipitation using antibodies, including commercially available antibodies. mRNA can be quantitated using methods that are well known and routine in the art, for example, using northern analysis, RNase protection, or polymerase chain reaction in connection with reverse transcription.

In specific embodiments, antibodies described herein specifically bind to KIT and inhibit cell proliferation by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art (e.g., BrdU incorporation assay). In specific embodiments, antibodies described herein specifically bind to KIT and inhibit cell proliferation by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art (e.g., BrdU incorporation assay).

In certain aspects, an anti-KIT antibody described herein which can act as an inhibitor of KIT activity can reduce or inhibit survival of cells that express KIT and that respond to KIT signaling (e.g., cells that proliferate in response to KIT ligand stimulation and KIT signaling). Cell survival assays are described in the art and can be readily carried out by one of skill in the art. For example, cell viability can be assessed by using trypan-blue staining or other cell death or viability markers known in the art. In a specific embodiment, the level of cellular ATP is measured to determined cell viability. In specific embodiments, cell viability is measured in three-day and seven-day periods using an assay standard in the art, such as the CellTiter-Glo Assay Kit (Promega) which measures levels of intracellular ATP. A reduction in cellular ATP is indicative of a cytotoxic effect. In another specific embodiment, cell viability can be measured in the neutral red uptake assay. In other embodiments, visual observation for morphological changes can include enlargement, granularity, cells with ragged edges, a filmy appearance, rounding, detachment from the surface of the well, or other changes. These changes are given a designation of T (100% toxic), PVH (partially toxic-very heavy-80%), PH (partially toxic-heavy-60%), P (partially toxic-40%), Ps (partially toxic-slight-20%), or 0 (no toxicity-0%), conforming to the degree of cytotoxicity seen. A 50% cell inhibitory (cytotoxic) concentration (IC₅₀) is determined by regression analysis of these data.

In specific embodiments, antibodies described herein specifically bind to KIT and inhibit cell survival by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art (e.g., trypan blue exclusion assay). In specific embodiments, antibodies described herein specifically bind to KIT and inhibit cell survival by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art (e.g., trypan blue assay).

In certain aspects, an anti-KIT antibody described herein, which can act as an inhibitor of KIT activity, is capable of inducing apoptosis (i.e., programmed cell death) of cells (e.g., MO7E cells) that express KIT and that respond to KIT signaling (e.g., cells that proliferate in response to KIT ligand stimulation and KIT signaling). Apoptosis are described in the art and can be readily carried out by one of skill in the art. For example, flow cytometry can be used to detect activated caspase 3, an apoptosis-mediating enzyme, in cells undergoing apoptosis, or Western blotting can be used to detect cleavage of poly(ADP-ribose) polymerase (PARP) (see, e.g., Smolich et al., Blood, 2001, 97:1413-1421). Cleavage of PARP is an indicator of apoptosis. In specific embodiments, antibodies described herein specifically bind to KIT and induce or enhance apoptosis by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art (e.g., flow cytometry to detect activated caspase 3). In specific embodiments, antibodies described herein specifically bind to KIT and induce or enhance apoptosis by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art (e.g., flow cytometry to detect activated caspase 3).

In certain aspects, an anti-KIT antibody described herein, which can act as an inhibitor of KIT activity, is capable of inhibiting or decreasing anchorage independent cell growth (e.g., colony formation) by cells (e.g., H526 cells or CHO cells expressing exogenous KIT) that express KIT and that respond to KIT signaling (e.g., cells that proliferate in response to KIT ligand stimulation and KIT signaling), as measured by methods commonly known in the art, e.g., soft agar assay. In specific embodiments, antibodies described herein specifically bind to KIT and inhibit or decrease anchorage independent cell growth by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art (e.g., soft agar assay). In specific embodiments, antibodies described herein specifically bind to KIT and inhibit or decrease anchorage independent cell growth by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art (e.g., soft agar assay).

Cells and cell lines which are appropriate for use in the assays described herein relating KIT activity are readily available (e.g., ATCC) or can be readily identified using methods known in the art. For example, cells and/or cell lines that express KIT endogenously or that possess KIT signaling or activity are known to one of skill in the art. In certain embodiments, cells or cell lines that are appropriate for use in the assays described herein can express KIT, either endogenously or recombinantly. In particular embodiments, cells or cell lines for use in cell proliferation assays can express KIT, endogenously or recombinantly, and proliferate or increase proliferation in response to KIT ligand (e.g., SCF) stimulation. Cells or cell lines for use in cell viability assays can express KIT, endogenously or recombinantly, and exert changes in cell viability in response to KIT ligand (e.g., SCF) stimulation. Cells or cell lines for use in apoptosis assays can express KIT, endogenously or recombinantly, and exert changes in apoptosis in response to KIT ligand (e.g., SCF) stimulation.

Non-limiting examples of cells that can be used in the methods and assays described herein include primary cells, transformed cells, stem cells, mast cells, primordial germ cells, oocytes, spermatocytes, embryonic stem cells, hematopoietic cells, erythroleukemia cells (e.g., F36P and TF-1 cell lines), human myeloid leukemia cell lines, such as MOTE cells; gastrointestinal stromal tumor cell lines such as ST-882 and GIST882; neuroblastoma cell lines such as SK-N-SH, SK-SY5Y, H-EP1, SK-N-BE(2), SK—N-BE(ZkM17), SK-N-BE(2)C, LA-N-1, or LA-N-1-5s; and small cell lung carcinoma cell lines such as ECC12, TMK1, MKN7, GCIY, and HGC27.

Alternatively, cells and cell lines that express KIT, e.g., human KIT, can routinely be generated recombinantly. Non-limiting examples of cells that can be engineered to express KIT recombinantly include COS cells, HEK 293 cells, CHO cells, fibroblasts (e.g., human fibroblasts), and MEFS.

In certain aspects, an anti-KIT antibody described herein, which can act as an inhibitor of KIT activity, is capable of inhibiting tumor grow or inducing tumor regression in mouse model studies. For example, tumor cell lines can be introduced into nude mice, and the mice can be administered with anti-KIT antibodies described herein one or more times, and tumor progression of the injected tumor cells can be monitored over a period of weeks and months. In some cases, administration of anti-KIT antibodies to the nude mice can occur prior to introduction of the tumor cell lines. Any appropriate tumor cell line (e.g., tumor cell line expressing KIT) can be used in the mouse xenograft models described herein. Non-limiting examples of tumor cells lines for use in these xenograft mouse models include megakaryoblastic leukemia cell lines such as MO7e; gastrointestinal stromal tumor cell lines such as ST-882 and GIST882, GIST48, GIST48B and GIST882; human erythroleukemic cell lines such as TF-1 and HEL; human promyelocytic leukemia cell line, HL60; neuroblastoma cell lines such as SK-N-SH, SK-SY5Y, H-EP1, SK-N-BE(2), SK-N-BE(ZkM17), SK-N-BE(2)C, LA-N-1, or LA-N-1-5s; and small cell lung carcinoma cell lines such as H526, DMS153, DMS79,ECC12, TMK1, MKN7, GCIY, and HGC27. In a specific embodiments, a tumor cell line for use in a xenograft mouse model is the GIST882, GIST430, GIST48, GIST48B, HEL, HL60, H526, DMS153, or DMS79 cell line. In certain embodiments, suitable cell lines for use in xenograft tumor models can be generated by recombinantly expressing KIT in cell. In specific embodiments, antibodies described herein specifically bind to KIT and inhibit tumor grow or induce tumor regression in a mouse model by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art. In specific embodiments, antibodies described herein specifically bind to KIT and inhibit tumor grow or induce tumor regression in a mouse model by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art. Determining tumor growth inhibition or tumor regression can be assessed by monitoring tumor size over a period of time, such as by physical measurement of palpable tumors, or other visual detection methods. For example, tumor cell lines can be generated to recombinantly express a visualization agent, such as green fluorescent protein (GFP) or luciferase, then in vivo visualization of GFP can be carried out by microscopy, and in vivo visualization of luciferase can be carried out by administering luciferase substrate to the xenograft mice and detecting luminescent due to the luciferase enzyme processing the luciferase substrate. The degree or level of detection of GFP or luciferase correlates to the size of the tumor in the xenograft mice.

In certain aspects, anti-KIT antibodies described herein bind specifically to KIT antigen and can increase survival of animals in tumor xenograft models. In specific embodiments, antibodies described herein specifically bind to KIT and increase survival of mice in tumor xenograft models by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art. In specific embodiments, antibodies described herein specifically bind to KIT and increase survival of mice in tumor xenograft models by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art. Survival can be determined by plotting a survival curve of number of surviving mice against time (e.g., days or weeks) after tumor cell line injection.

Provided herein are antibodies that immunospecifically bind a KIT polypeptide, e.g., a human KIT polypeptide, e.g., a D4/D5 region of KIT, for example, human KIT, with a particular affinity.

“Affinity” of an antibody described herein for an epitope (e.g., KIT epitope) is a term well understood in the art and refers to the extent, or strength, of binding of an antibody to an epitope. Affinity can be measured and/or expressed in a number of ways known in the art, including, but not limited to, equilibrium dissociation constant (KD or Kd), apparent equilibrium dissociation constant (KD′ or Kd′), and IC₅₀ (amount needed to effect 50% inhibition in a competition assay). It is understood that, for purposes described herein, an affinity is an average affinity for a given population of antibodies which bind to an epitope. Values of KD′ described herein in terms of milligram (mg) Ig per mL or mg/mL indicate mg Ig per mL of serum, although plasma can be used. When antibody affinity is used as a basis for administration of the treatment methods described herein, or selection for the treatment methods described herein, antibody affinity can be measured before and/or during treatment, and the values obtained can be used by a clinician in assessing whether a human patient is an appropriate candidate for treatment.

In specific aspects, provided herein are antibodies that have a high binding affinity (e.g., antibodies having a K_{D} of less than 100 nM, 50 nM, 10 nM, 1 nM, 100 pM, or 50 pM) for a KIT antigen, preferably a human KIT antigen, in particular the D4/D5 region of a human KIT. In a specific embodiment, an antibody described herein has an association rate constant or kₒₙ rate (antibody (Ab)+antigen (Ag)^{kon}→Ab−Ag) of at least 2×10⁵M⁻¹s⁻¹, at least 5×10⁵ M⁻¹s⁻¹, at least 10⁶ M⁻¹s⁻¹, at least 5×10⁶ M⁻¹s, at least 10⁷M⁻¹s⁻¹, at least 5×10⁷M⁻¹s⁻¹, or at least 10⁸ M⁻¹s⁻¹. In a certain embodiment, an antibody described herein has a kₒₙ of at least 2×10⁵M⁻¹s⁻¹, at least 5×10⁵M⁻¹s⁻¹, at least 10⁶ M⁻¹s⁻¹, at least 5×10⁶M⁻¹s⁻¹, at least 10⁷M⁻¹s⁻¹, at least 5 X 10⁷M⁻¹s⁻¹, or at least 10⁸M⁻¹s⁻¹.

In another embodiment, an antibody described herein has a k_{off} rate ((Ab−Ag)^{koff}→antibody (Ab)+antigen) of less than 10⁻¹s⁻¹, less than 5×10⁻¹s⁻¹, less than 10⁻² s⁻¹, less than 5×10⁻² s⁻¹, less than 10⁻³ s⁻¹, less than 5×10⁻³ s⁻¹, less than 10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷s⁻¹, less than 5×10⁻⁷s⁻¹, less than 10⁻⁸ s⁻¹, less than 5×10⁻⁸s⁻¹, less than 10⁻⁹s⁻¹, less than 5 X 10⁻⁹ s⁻¹, or less than 10⁻¹⁰ s⁻¹. In a specific embodiment, an antibody described herein has a kₒₙ of less than 5×10⁻⁴s⁻¹, less than 10⁻⁵ s⁻¹, less than 5×10⁻⁵s⁻¹, less than 10⁻⁶s⁻¹, less than 5×10⁻⁶ s⁻¹, less than 10⁻⁷ s⁻¹, less than 5×10⁻⁷s⁻¹, less than 10⁻⁸s⁻¹, less than 5×10⁻⁸s⁻¹, less than 10⁻⁹ s⁻¹, less than 5×10⁻⁹s⁻¹, or less than 10⁻¹° s⁻¹.

In another embodiment, an antibody described herein has an affinity constant or Kₐ (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M.

In a particular embodiment, an antibody described herein has a dissociation constant or K_{D} (k_{off}/kₒₙ) of less than 10⁻² M, less than 5×10⁻² M, less than 10⁻³ M, less than 5×10⁻³ M, less than 10⁻⁴ M, less than 5×10⁻⁴ M, less than 10⁻⁵ M, less than 5×10⁻⁵ M, less than 10⁻⁶ M, less than 5×10⁻⁶ M, less than 10⁻⁷ M, less than 5×10⁻⁷M, less than 10⁻⁸M, less than 5×10⁻⁸ M, less than 10⁻⁹ M, less than 5×10⁻⁹ M, less than 10⁻¹⁰ M, less than 5×10⁻¹⁰M, less than 10⁻¹¹ M, less than 5×10⁻¹¹ M, less than 10⁻¹² M, less than 5×10⁻¹²M, less than 10⁻¹³M, less than 5×10⁻¹³ M, less than 10⁻¹⁴M, less than 5×10⁻¹⁴M, less than 10⁻¹⁵ M, or less than 5×10⁻¹⁵ M.

In specific embodiments, an antibody (e.g., human antibody) immunospecifically binds to a KIT antigen (e.g., a D4/D5 region of KIT, for example human KIT), and has a dissociation constant (K_{D}) of less than 500,000 pM (500 nM), 100,000 pM (100 nM), 50,000 pM (50 nM), 10,000 pM (10 nM), 3,000 pM (3 nM), less than 2,500 pM (2.5 nM), less than 2,000 pM, less than 1,500 pM, less than 1,000 pM, less than 750 pM, less than 500 pM, less than 250 pM, less than 200 pM, less than 150 pM, less than 100 pM, less than 75 pM as assessed using an assay described herein or known to one of skill in the art (e.g., a Biacore™ assay) (Biacore™ International AB, Uppsala, Sweden). In a specific embodiment, the antibodies described herein (e.g., antibody Ab1 or Ab21, an antibody which binds to the same epitope as that of antibody Ab1 or Ab21, or an antibody comprising the CDRs of Ab1 or Ab21) immunospecifically bind to a KIT antigen (e.g., a D4/D5 region of KIT, for example human KIT), and have a K_{D} in the range of from 25 to 100,000 pM, 25 to 75,000 pM, 25 to 50,000 pM, 25 to 40,000 pM, 25 to 30,000 pM, 25 to 20,000 pM, 25 to 10,000 pM, 25 to 1,000 pM, 25 to 500 pM, 25 to 250 pM, 25 to 100 pM, or 25 to 50 pM as assessed using methods described herein or known to one of skill in the art (e.g., a Biacore™ assay, assay using KinExA 3000 instrument). In a particular embodiment, antibodies described herein (e.g., antibody Ab1 or Ab21, an antibody which binds to the same epitope as that of antibody Ab1 or Ab21, or an antibody comprising the CDRs of Ab1 or Ab21) immunospecifically bind to a KIT antigen (e.g., a D4/D5 region of KIT, for example human KIT), and have a K_{D} of about 1 nM to about 25 nM, or any value in between, as assessed using methods described herein or known to one of skill in the art (e.g., a Biacore™ assay, assay using KinExA 3000 instrument). In a particular embodiment, antibodies described herein (e.g., antibody Ab1 or Ab21, an antibody which binds to the same epitope as that of antibody Ab1 or Ab21, or an antibody comprising the CDRs of Ab1 or Ab21) immunospecifically bind to a KIT antigen (e.g., a D4/D5 region of KIT, for example human KIT), and have a K_{D} of about 1 nM to about 25 nM, or any value in between, as assessed using methods described herein or known to one of skill in the art (e.g., a Biacore™ assay, assay using KinExA 3000 instrument). In a particular embodiment, antibodies described herein (e.g., antibody Ab1 or Ab21, an antibody which binds to the same epitope as that of antibody Ab1 or Ab21, or an antibody comprising the CDRs of Ab1 or Ab21) immunospecifically bind to KIT antigen, (e.g., a D4/D5 region of KIT, for example human KIT), and have a K_{D} of about 1 nM, 1.5 nM, 2 nM, 2.5 nM, 3 nM, 3.5 nM, 4 nM, 4.5 nM, 5 nM, 5.5 nM, 6 nM, 6.5 nM, 7 nM, 8 nM, 9 nM, 10 nM, 11 nM, 12 nM, 13 nM, 14 nM, 15 nM, 16 nM, 17 nM, 18 nM, 19 nM, 20 nM, or 21 nM, as assessed using methods described herein or known to one of skill in the art (e.g., a Biacore™ assay, assay using KinExA 3000 instrument). In a particular embodiment, antibodies described herein immunospecifically bind to KIT antigen (e.g., a D4/D5 region of KIT, for example human KIT), and have a K_{D} from about 100 pM to about 10 nM, as assessed using methods described herein or known to one of skill in the art (e.g., ELISA, assay using KinExA 3000 instrument, or Biacore™ assay). In a particular embodiment, antibody Ab1 or Ab21, an antibody which binds to the same epitope as that of antibody Ab1 or Ab21, or an antibody comprising the CDRs of Ab1 or Ab21, has a K_{D} from about 100 pM to about 10 nM, as assessed using methods described herein or known to one of skill in the art (e.g., ELISA, assay using KinExA 3000 instrument, or Biacore™ assay).

In specific embodiments, an anti-KIT antibody (e.g., human antibody) immunospecifically binds to a KIT antigen (e.g., a D4/D5 region of KIT, for example human KIT), and has a concentration at 50% binding to antigen of less than 3000 pM (3 nM), less than 2500 pM (2.5 nM), less than 2000 pM, less than 1500 pM, less than 1000 pM, less than 750 pM, less than 500 pM, less than 250 pM, less than 200 pM, less than 150 pM, less than 100 pM, less than 75 pM as assessed using an assay described herein or known to one of skill in the art (e.g., solid phase ELISA as described in section 6). In a specific embodiment, an antibody described herein immunospecifically binds to a KIT antigen (e.g., a D4/D5 region of KIT, for example human KIT), and has a concentration at 50% binding to antigen in the range of from 25 to 500,000 pM (500 nM), 25 to 250,000 pM (250 nM), 25 to 100,000 pM (100 nM), 25 to 75,000 pM (75 nM), 25 to 50,000 pM (50 nM), 25 to 40,000 pM (40 nM), 25 to 30,000 pM (30 nM), 25 to 20,000 pM (20 nM), 25 to 10,000 pM (10 nM), 25 to 1,000 pM (1 nM), 25 to 500 pM, 25 to 250 pM, 25 to 100 pM, or 25 to 50 pM as assessed using methods described herein or known to one of skill in the art (e.g., solid phase ELISA as described in section 6). In a particular embodiment, an antibody described herein immunospecifically binds to a KIT antigen (e.g., a D4/D5 region of KIT, for example human KIT), and has a concentration at 50% binding to antigen of about 1 nM to about 25 nM, or any value in between, as assessed using methods described herein or known to one of skill in the art (e.g., solid phase ELISA as described in section 6). In a particular embodiment, and antibody described herein immunospecifically binds to a KIT antigen (e.g., a D4/D5 region of KIT, for example human KIT), and has a concentration at 50% binding to antigen of about 1 nM to about 25 nM, or any value in between, as assessed using methods described herein or known to one of skill in the art (e.g., solid phase ELISA as described in section 6). In a particular embodiment, an antibody described herein immunospecifically binds to KIT antigen (e.g., a D4/D5 region of KIT, for example human KIT), and has a concentration at 50% binding of about 1 nM, 1.5 nM, 2 nM, 2.5 nM, 3 nM, 3.5 nM, 4 nM, 4.5 nM, 5 nM, 5.5 nM, 6 nM, 6.5 nM, 7 nM, 8 nM, 9 nM, 10 nM, 11 nM, 12 nM, 13 nM, 14 nM, 15 nM, 16 nM, 17 nM, 18 nM, 19 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, or 500 nM as assessed using methods described herein or known to one of skill in the art (e.g., solid phase ELISA as described in section 6). In a particular embodiment, an antibody described herein (e.g., antibody Ab1 or Ab21, an antibody which binds to the same epitope as that of antibody Ab1 or Ab21, or an antibody comprising the CDRs of Ab1 or Ab21) immunospecifically binds to KIT antigen (e.g., a D4/D5 region of KIT, for example human KIT), and has a concentration at 50% binding from about 100 pM to about 10 nM, or from about 100 pM to about 500 pM, as assessed using methods described herein or known to one of skill in the art (e.g., ELISA, assay using KinExA 3000 instrument, or Biacore™ assay). In a particular embodiment, an antibody described herein (e.g., antibody Ab1 or Ab21, an antibody which binds to the same epitope as that of antibody Ab1 or Ab21, or an antibody comprising the CDRs of Ab1 or Ab21) immunospecifically binds to KIT antigen (e.g., a D4/D5 region of KIT, for example human KIT), and has a concentration at 50% binding from about 150 pM to about 200 pM, or any value in between (e.g., 155 pM, 156 pM, 170 pM, or 171 pM), as assessed using methods described herein or known to one of skill in the art (e.g., ELISA, assay using KinExA 3000 instrument, or Biacore™ assay). In a particular embodiment, an antibody described herein (e.g., antibody Ab1 or Ab21, an antibody which binds to the same epitope as that of antibody Ab1 or Ab21, or an antibody comprising the CDRs of Ab1 or Ab21) immunospecifically binds to KIT antigen (e.g., a D4/D5 region of KIT, for example human KIT), and has a concentration at 50% binding of less than about 200 pM, as assessed using methods described herein or known to one of skill in the art (e.g., ELISA, assay using KinExA 3000 instrument, or Biacore™ assay).

In specific embodiments, an anti-KIT antibody which is an antigen-binding fragment of a whole or entire antibody, e.g., Fab fragment, has comparable affinity to KIT relative to the affinity of the whole or entire anti-KIT antibody. In certain embodiments, an anti-KIT antibody which is an antigen-binding fragment of a whole or entire antibody, e.g., Fab fragment, specifically binds to KIT and has a K_{D} that is comparable to the K_{D} of the whole or entire anti-KIT antibody as assessed using methods described herein or known to one of skill in the art (e.g., a Biacore™ assay, assay using KinExA 3000 instrument). In certain embodiments, an anti-KIT antibody which is an antigen-binding fragment of a whole or entire antibody, e.g., Fab fragment, specifically binds to KIT and has a K_{D} that is less than the K_{D} of the whole or entire anti-KIT antibody as assessed using methods described herein or known to one of skill in the art (e.g., a Biacore™ assay, assay using KinExA 3000 instrument). In particular embodiments, an anti-KIT antibody which is an antigen-binding fragment of a whole or entire antibody, e.g., Fab fragment, specifically binds to KIT and has a K_{D} that is about 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65%, 70%, 75%, 80%, 85%, or 90% less than the K_{D} of the whole or entire anti-KIT antibody as assessed using methods described herein or known to one of skill in the art (e.g., a Biacore™ assay, assay using KinExA 3000 instrument). In particular embodiments, an anti-KIT antibody which is an antigen-binding fragment of a whole or entire antibody, e.g., Fab fragment, specifically binds to KIT and has a K_{D} that is at most about 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65%, 70%, 75%, 80%, 85%, or 90% less than the K_{D} of the whole or entire anti-KIT antibody as assessed using methods described herein or known to one of skill in the art (e.g., a Biacore™ assay, assay using KinExA 3000 instrument). In particular embodiments, an anti-KIT antibody which is an antigen-binding fragment of a whole or entire antibody, e.g., Fab fragment, specifically binds to KIT and has a K_{D} that is at least about 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65%, 70%, 75%, 80%, 85%, or 90% less than the K_{D} of the whole or entire anti-KIT antibody as assessed using methods described herein or known to one of skill in the art (e.g., a Biacore™ assay, assay using KinExA 3000 instrument).

In certain embodiments, an anti-KIT antibody which is an antigen-binding fragment of the native (or entire) antibody, e.g., Fab fragment, specifically binds to KIT and has a K_{D} that is more than the K_{D} of the native (or entire) anti-KIT antibody as assessed using methods described herein or known to one of skill in the art (e.g., a Biacore™ assay, assay using KinExA 3000 instrument). In particular embodiments, an anti-KIT antibody which is an antigen-binding fragment of a whole or entire antibody, e.g., Fab fragment, specifically binds to KIT and has a K_{D} that is about 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65%, 70%, 75%, 80%, 85%, 90%, or 95% higher than the K_{D} of the whole or entire anti-KIT antibody as assessed using methods described herein or known to one of skill in the art (e.g., a Biacore™ assay, assay using KinExA 3000 instrument). In particular embodiments, an anti-KIT antibody which is an antigen-binding fragment of a whole or entire antibody, e.g., Fab fragment, specifically binds to KIT and has a K_{D} that is at most about 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65%, 70%, 75%, 80%, 85%, 90%, or 95% higher than the K_{D} of the whole or entire anti-KIT antibody as assessed using methods described herein or known to one of skill in the art (e.g., a Biacore™ assay, assay using KinExA 3000 instrument). In particular embodiments, an anti-KIT antibody which is an antigen-binding fragment of a whole or entire antibody, e.g., Fab fragment, specifically binds to KIT and has a K_{D} that is at least about 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65%, 70%, 75%, 80%, 85%, 90%, or 95% higher than the K_{D} of the whole or entire anti-KIT antibody as assessed using methods described herein or known to one of skill in the art (e.g., a Biacore™ assay, assay using KinExA 3000 instrument). In particular embodiments, an anti-KIT antibody which is an antigen-binding fragment of a whole or entire antibody, e.g., Fab fragment, specifically binds to KIT and has a K_{D} that is at most about 1 fold, 1.1 fold, 1.2 fold, 1.3 fold, 1.4 fold, 1.5 fold, 1.6 fold, 1.7 fold, 1.8 fold, 1.9 fold, 2 fold, 2.5 fold, 3 fold, 3.5 fold, 4 fold, 4.5 fold or 5 fold higher than the K_{D} of the whole or entire anti-KIT antibody as assessed using methods described herein or known to one of skill in the art (e.g., a Biacore™ assay, assay using KinExA 3000 instrument).

Methods for determining affinity of an antibody to its target antigen are readily available and described in the art. For example, the affinities and binding properties of an antibody for its target antigen, can be determined by a variety of in vitro assay methods (biochemical or immunological based assays) known in the art such as equilibrium methods (e.g., enzyme-linked immunoabsorbent assay (ELISA), or radioimmunoassay (RIA)), or kinetics (e.g., Biacore™ analysis), and other methods such as indirect binding assays, competitive inhibition assays, fluorescence resonance energy transfer (FRET), immunoprecipitation, gel electrophoresis and chromatography (e.g., gel filtration). These and other methods can utilize a label on one or more of the components being examined and/or employ a variety of detection methods including but not limited to chromogenic, fluorescent, luminescent, or isotopic labels. In certain embodiments, use of labels is not necessary, e.g., Biacore™ systems utilize the natural phenomenon of surface plasmon resonance (SPR) to deliver data in real time, without the use of labels. A detailed description of binding affinities and kinetics can be found in Paul, W. E., ed., Fundamental Immunology, 4th Ed. (Lippincott-Raven, Philadelphia 1999), which focuses on antibody-immunogen interactions.

In certain aspects, the affinity of an antibody described herein for a KIT antigen, e.g., human KIT, for example a D4/D5 region of KIT (e.g., human KIT), can be characterized indirectly using cell-based assays. For example, cells expressing KIT on their cell membrane surface can be contacted with anti-KIT antibodies, and cellular activities downstream of KIT can be determined using assays known in the art. For examples, phosphorylation of the cytoplasmic domain of KIT can be determined by immunoblotting (or Western blotting) following contacting the cells with an anti-KIT antibody; cellular extracts are obtained and processed for immunoblotting (e.g., subjecting the cellular extracts to sodium dodecyl sulfate polyacrylamide gel electrophoresis (SDS-PAGE) and transferring the proteins separated on the gel to a membrane (e.g., nitrocellulose or polyvinylidene fluoride (PVDF)) with an antibody that specifically binds to a phosphorylated tyrosine in the cytoplasmic domain of KIT, but does not bind an unphosphorylated tyrosine.

In certain embodiments, an anti-KIT antibody described herein specifically binds to a KIT antigen, e.g., human KIT, for example a D4/D5 region of KIT (e.g., human KIT), and induces or enhances dimerization and phosphorylation of KIT, in the presence or absence of the KIT ligand SCF. In some embodiments, an anti-KIT antibody described herein can inhibit or decrease KIT ligand, e.g., SCF, binding to KIT (i.e., an anti-KIT antibody can compete with a KIT ligand, e.g., SCF, for binding to KIT). In such case, cells can be contacted with an anti-KIT antibody and a KIT ligand, and the degree of inhibition of KIT phosphorylation can be determined as an indication of the degree of the anti-KIT antibody competing with the KIT ligand for binding to KIT.

Antibodies provided herein (e.g., antibody Ab1 or Ab21, an antibody comprising the CDRs of antibody Ab1 or Ab21, or an antibody which binds to the same epitope as that of antibody Ab1 or Ab21) include, but are not limited to, monoclonal antibodies, recombinantly produced antibodies, multispecific antibodies (including bi-specific antibodies), human antibodies, humanized antibodies, chimeric antibodies, synthetic antibodies, intrabodies, heteroconjugate antibodies, single domain antibodies, monovalent antibodies, single-chain Fvs (scFv) (e.g., including monospecific, bispecific, etc.), camelized antibodies, affybodies, Fab fragments, F(ab′) fragments, disulfide-linked Fvs (sdFv), anti-idiotypic (anti-Id) antibodies (including, e.g., anti-anti-Id antibodies), and epitope-binding fragments/antigen binding fragment of any of the above. In a particular embodiment, an antibody provided herein (e.g., antibody Ab1 or Ab21, an antibody comprising the CDRs of antibody Ab1 or Ab21, or an antibody which binds to the same epitope as that of antibody Ab1 or Ab21) is a Fab fragment that immunospecifically binds to a KIT polypeptide, such as the D4/D5 region of KIT. In a specific embodiment, antibodies described herein (e.g., antibody Ab1 or Ab21, an antibody comprising the CDRs of antibody Ab1 or Ab21, or an antibody which binds to the same epitope as that of antibody Ab1 or Ab21) are monoclonal antibodies or isolated monoclonal antibodies. Monoclonal antibodies may include humanized antibodies, human antibodies, and chimeric antibodies. In another specific embodiment, an antibody described herein is a humanized monoclonal antibody. In yet another specific embodiment, an antibody described herein (e.g., antibody Ab1 or Ab21, an antibody comprising the CDRs of antibody Ab1 or Ab21, or an antibody which binds to the same epitope as that of antibody Ab1 or Ab21) is a human monoclonal antibody. In a particular embodiment, an antibody described herein (e.g., antibody Ab1 or Ab21, an antibody comprising the CDRs of antibody Ab1 or Ab21, or an antibody which binds to the same epitope as that of antibody Ab1 or Ab21) is a recombinant antibody, for example, a recombinant human antibody or a recombinant monoclonal antibody. In a particular embodiment, an antibody described herein is not a humanized antibody. In another particular embodiment, an antibody described herein is not a murine antibody. In certain embodiments, an antibody described herein does not contain non-human amino acid sequences (e.g., non-human CDRs or non-human framework regions). In a particular embodiment, an antibody described herein (e.g., antibody Ab1 or Ab21, an antibody comprising the CDRs of antibody Ab1 or Ab21, or an antibody which binds to the same epitope as that of antibody Ab1 or Ab21) is a polypeptide comprising one, two or three VL CDRs and/or one, two or three VH CDRs of antibody Ab1 or Ab21, or of any one of antibodies Ab2-Ab20, Ab24-Ab36, and Ab38-Ab192, and wherein said antibody immunospecifically binds to a KIT polypeptide, such as the D4/D5 region of KIT.

Antibodies provided herein (e.g., antibody Ab1 or Ab21, an antibody comprising the CDRs of antibody Ab1 or Ab21, or an antibody which binds to the same epitope as that of antibody Ab1 or Ab21) include immunoglobulin molecules of any type (e.g., IgG, IgE, IgM, IgD, IgA and IgY), class (e.g., IgG1, IgG2, IgG3, IgG4, IgA1 and IgA2) or subclass of immunoglobulin molecule. In a specific embodiment, an antibody provided herein (e.g., antibody Ab1 or Ab21, an antibody comprising the CDRs of antibody Ab1 or Ab21, or an antibody which binds to the same epitope as that of antibody Ab1 or Ab21) is an IgG antibody (e.g., human IgG antibody), or a class (e.g., human IgG1 or IgG4) or subclass thereof. In another specific embodiment, an antibody described herein is an IgG1 (e.g., human IgG1 (isotype a, z, or f)) or IgG4 antibody. In certain embodiments, an antibody described herein is a whole or entire antibody, e.g., a whole or entire human antibody.

Antibodies provided herein can include antibody fragments that retain the ability to specifically bind to an epitope, i.e., KIT epitope (e.g., a KIT epitope within a KIT polypeptide containing the D4/D5 region of human KIT (V308 to H15, SEQ ID NO: 171)). In a specific embodiment, fragments include Fab fragments (an antibody fragment that contains the antigen-binding domain and comprises a light chain and part of a heavy chain (i.e., the VH and CH1 domains of a heavy chain) bridged by a disulfide bond); Fab′ (an antibody fragment containing a single antigen-binding domain comprising an Fab and an additional portion of the heavy chain through the hinge region); F(ab′)₂ (two Fab′ molecules joined by interchain disulfide bonds in the hinge regions of the heavy chains; the Fab′ molecules can be directed toward the same or different epitopes); a bispecific Fab (a Fab molecule having two antigen binding domains, each of which can be directed to a different epitope); a single chain Fab chain comprising a variable region, also known as, a sFv (the variable, antigen-binding determinative region of a single light and heavy chain of an antibody linked together by a chain of 10-25 amino acids); a disulfide-linked Fv, or dsFv (the variable, antigen-binding determinative region of a single light and heavy chain of an antibody linked together by a disulfide bond); a camelized VH (the variable, antigen-binding determinative region of a single heavy chain of an antibody in which some amino acids at the VH interface are those found in the heavy chain of naturally occurring camel antibodies); a bispecific sFv (a sFv or a dsFv molecule having two antigen-binding domains, each of which can be directed to a different epitope); a diabody (a dimerized sFv formed when the VH domain of a first sFv assembles with the VL domain of a second sFv and the VL domain of the first sFv assembles with the VH domain of the second sFv; the two antigen-binding regions of the diabody can be directed towards the same or different epitopes); and a triabody (a trimerized sFv, formed in a manner similar to a diabody, but in which three antigen-binding domains are created in a single complex; the three antigen binding domains can be directed towards the same or different epitopes). Antibodies provided herein can also include one or more CDR sequences of an antibody. The CDR sequences can be linked together on a scaffold when two or more CDR sequences are present. In certain embodiments, an antibody comprises a single-chain Fv (“scFv”). scFvs are antibody fragments comprising the VH and VL domains of an antibody, wherein these domains are present in a single polypeptide chain. Generally, the scFv polypeptide further comprises a polypeptide linker between the VH and VL domains which enables the scFv to form the desired structure for antigen binding. For a review of scFvs, see Pluckthun in The Pharmacology of Monoclonal Antibodies, vol. 113, Rosenburg and Moore eds. Springer-Verlag, New York, pp. 269-315 (1994). Without being bound by any particular theories, Fv molecules can be able to penetrate tissues because of their small size. A whole antibody can be enzymatically cleaved by pepsin to produce a F(ab′)₂ fragment, or can be enzymatically cleaved by papain to produce two Fab fragments.

The antibodies described herein can be from any animal origin including birds (e.g., chicken or rooster) and mammals (e.g., human, murine, donkey, sheep, rabbit, goat, guinea pig, camel, dog, cat, pig, rat, monkey, cow, or horse). In certain embodiments, the antibodies described herein are human or humanized monoclonal antibodies.

In certain aspects, provided herein are antibodies that have been modified by affinity maturation. An affinity matured antibody is one with one or more alterations in one or more variable regions (e.g., framework regions or CDRs) and/or hypervariable regions thereof which result in an improvement in the affinity of the antibody for antigen, compared to a parent antibody which does not possess those alteration(s). Certain affinity matured antibodies can have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al., Proc. Nat. Acad. Sci. USA 91:3809-3813 (1994); Schier et al., Gene 169: 147-155 (1995); Yelton et al., J. Immunol. 155: 1994-2004 (1995); Jackson et al., J. Immunol. 154(7):3310-9 (1995); and Hawkins et al., J. Mol. Biol. 226:889-896 (1992). In certain embodiments, an antibody described herein is an affinity matured antibody.

Antibodies provided herein can include antibodies comprising chemical modifications, for example, antibodies which have been chemically modified, e.g., by covalent attachment of any type of molecule to the antibody. For example, but not by way of limitation, an anti-KIT antibody can be glycosylated, acetylated, pegylated, phosphorylated, or amidated, can be derivitized via protective/blocking groups, or can further comprise a cellular ligand and or other protein or peptide, etc. For example, an antibody provided herein can be chemically modified, e.g., by glycosylation, acetylation, pegylation, phosphorylation, amidation, derivatization by known protecting/blocking groups, proteolytic cleavage, linkage to a cellular ligand or other protein, etc. Further, an anti-KIT antibody described herein can contain one or more non-classical amino acids.

In a particular embodiment, provided herein is an anti-KIT antibody which has been modified in a manner suitable for large scale manufacturing. For example, the BI-HEX® technology platform (Boehringer Ingleheim, Germany) can be used to adapt the anti-KIT antibodies described herein for suitable large scale manufacturing in recombinant mammalian cell expression systems. Such adaptation can involve cloning polynucleotide sequences encoding the necessary domains of an anti-KIT antibody, such as one or more CDRs or FRs, into a suitable expression vector which also contains polynucleotide sequences encoding suitable constant regions, so that an entire antibody is produced. The polynucleotide sequences provided by the expression vectors are nucleotide sequences which can have been optimized to maximize antibody yield and stability for cell culture manufacturing conditions and purification processes.

5.1.1. Conjugates

In some embodiments, provided herein are antibodies (e.g., antibody Ab1 or Ab21, an antibody comprising the CDRs of antibody Ab1 or Ab21, or an antibody which binds to the same epitope as that of antibody Ab1 or Ab21, or an antigen binding fragment of such antibody) conjugated or recombinantly fused to a diagnostic, detectable or therapeutic agent or any other molecule. The conjugated or recombinantly fused antibodies can be useful, e.g., for monitoring or prognosing the onset, development, progression and/or severity of a KIT-mediated disorder or disease as part of a clinical testing procedure, such as determining the efficacy of a particular therapy. Antibodies described herein can also be conjugated to a molecule (e.g., polyethylene glycol) which can affect one or more biological and/or molecular properties of the antibodies, for example, stability (e.g., in serum), half-life, solubility, and antigenicity. In a specific embodiment, the conjugated antibody comprises Ab1 or Ab21, or a KIT-binding portion thereof, e.g., any such portion described herein. In a specific embodiment, the conjugated antibody, or a KIT-binding portion thereof, comprises the CDRs of Ab1 or Ab21. In a specific embodiment, the conjugated antibody comprises VL CDR1, VL CDR2, and VL CDR3 having the amino acid sequences of SEQ ID NO: 2, SEQ ID NO: 14, and SEQ ID NO: 26, respectively. In a specific embodiment, the conjugated antibody comprises VH CDR1, VH CDR2, and VH CDR3 having the amino acid sequences of SEQ ID NO: 86, SEQ ID NO: 98, and SEQ ID NO: 110, respectively. In a specific embodiment, the conjugated antibody comprises VL CDR1, VL CDR2, and VL CDR3 having the amino acid sequences of SEQ ID NO: 330, SEQ ID NO: 331, and SEQ ID NO: 332, respectively. In a specific embodiment, the conjugated antibody comprises VH CDR1, VH CDR2, and VH CDR3 having the amino acid sequences of SEQ ID NO: 337, SEQ ID NO: 338, and SEQ ID NO: 339, respectively.

Such diagnosis and detection can be accomplished, for example, by coupling the antibody to detectable molecules or substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (¹³¹I, ¹²⁵I, ¹²³I, and ¹²¹I), carbon (¹⁴C), sulfur (³⁵S), tritium (³H), indium (¹¹⁵In, ¹¹³_{In}, ¹¹²In, and ¹¹¹In), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵ST, ³²P, ¹⁵³Gd, ¹⁶⁹Yb, ⁵¹Cr, ⁵⁴Mn, ⁷⁵Se, ¹¹³Sn, and ¹¹⁷Sn; and positron emitting metals using various positron emission tomographies, and non-radioactive paramagnetic metal ions.

Provided are antibodies described herein conjugated or recombinantly fused to a therapeutic moiety (or one or more therapeutic moieties) and uses of such antibodies. The antibody can be conjugated or recombinantly fused to a therapeutic moiety, such as a toxin or cytotoxin, e.g., a cytostatic or cytocidal agent, a therapeutic agent or a radioactive metal ion, e.g., alpha-emitters. A cytotoxin or cytotoxic agent includes any agent that is detrimental to cells. Therapeutic moieties include, but are not limited to, antimetabolites (e.g., methotrexate, 6-mercaptopurine, 6-thioguanine, cytarabine, 5-fluorouracil decarbazine); alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP), and cisplatin); anthracyclines (e.g., daunorubicin (formerly daunomycin) and doxorubicin); antibiotics (e.g., d actinomycin (formerly actinomycin), bleomycin, mithramycin, and anthramycin (AMC)); Auristatin molecules (e.g., auristatin PHE, bryostatin 1, and solastatin 10; see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999), all of which are incorporated herein by reference); hormones (e.g., glucocorticoids, progestins, androgens, and estrogens), DNA-repair enzyme inhibitors (e.g., etoposide or topotecan), kinase inhibitors (e.g., compound ST1571, imatinib mesylate (Kantarjian et al., Clin Cancer Res. 8(7):2167-76 (2002)); cytotoxic agents (e.g., paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucorticoids, procaine, tetracaine, lidocaine, propranolol, and puromycin and analogs or homologs thereof and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459); farnesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305); topoisomerase inhibitors (e.g., camptothecin; irinotecan; SN-38; topotecan; 9-aminocamptothecin; GG-211 (GI 147211); DX-8951f; IST-622; rubitecan; pyrazoloacridine; XR-5000; saintopin; UCE6; UCE1022; TAN-1518A; TAN 1518B; KT6006; KT6528; ED-110; NB-506; ED-110; NB-506; and rebeccamycin); bulgarein; DNA minor groove binders such as Hoescht dye 33342 and Hoechst dye 33258; nitidine; fagaronine; epiberberine; coralyne; beta-lapachone; BC-4-1; bisphosphonates (e.g., alendronate, cimadronte, clodronate, tiludronate, etidronate, ibandronate, neridronate, olpandronate, risedronate, piridronate, pamidronate, zolendronate) HMG-CoA reductase inhibitors, (e.g., lovastatin, simvastatin, atorvastatin, pravastatin, fluvastatin, statin, cerivastatin, lescol, lupitor, rosuvastatin and atorvastatin); antisense oligonucleotides (e.g., those disclosed in the U.S. Pat. Nos. 6,277,832, 5,998,596, 5,885,834, 5,734,033, and 5,618,709); adenosine deaminase inhibitors (e.g., Fludarabine phosphate and 2-Chlorodeoxyadenosine); ibritumomab tiuxetan (Zevalin®); tositumomab (Bexxar®)) and pharmaceutically acceptable salts, solvates, clathrates, and prodrugs thereof.

In particular embodiments, a drug moiety is an antitubulin drug, such as an auristatin or a derivative thereof. Non-limiting examples of auristatins include monomethyl auristatin E (MMAE), monomethyl auristatin F (MMAF), auristatin PYE, and auristatin E (AE). In certain embodiments, a drug moiety is a microtubule-disrupting agent such as maytansine or a derivative thereof, e.g., maytansinoid DM1 or maytansinoid DM4. In certain embodiments, a drug moiety is a prodrug, e.g., a prodrug of a CC-1065 (rachelmycin) analogue. In a specific embodiment, and antibody described herein (e.g., antibody Ab1 or Ab21, an antibody comprising the CDRs of antibody Ab1 or Ab21, or an antibody which binds to the same epitope as that of antibody Ab1 or Ab21) is conjugated to an antitubulin drug, such as an auristatin or a derivative thereof (e.g., monomethyl auristatin E (MMAE), monomethyl auristatin F (MMAF), auristatin PYE, and auristatin E (AE)); to a microtubule-disrupting agent, for example, maytansine or a derivative thereof, e.g., maytansinoid DM1; or to a prodrug, e.g., a prodrug of a CC-1065 (rachelmycin) analogue.

Further, an antibody described herein can be conjugated or recombinantly fused to a therapeutic moiety or drug moiety that modifies a given biological response. Therapeutic moieties or drug moieties are not to be construed as limited to classical chemical therapeutic agents. For example, the drug moiety can be a protein, peptide, or polypeptide possessing a desired biological activity. Such proteins can include, for example, a toxin such as abrin, ricin A, pseudomonas exotoxin, cholera toxin, or diphtheria toxin; a protein such as tumor necrosis factor, γ-interferon, α-interferon, nerve growth factor, platelet derived growth factor, tissue plasminogen activator, an apoptotic agent, e.g., TNF-γ, TNF-γ, AIM I (see, International Publication No. WO 97/33899), AIM II (see, International Publication No. WO 97/34911), Fas Ligand (Takahashi et al., 1994, J. Immunol., 6:1567-1574), and VEGF (see, International Publication No. WO 99/23105), an anti-angiogenic agent, e.g., angiostatin, endostatin or a component of the coagulation pathway (e.g., tissue factor); or, a biological response modifier such as, for example, a lymphokine (e.g., interferon gamma, interleukin-1 (“IL-1”), interleukin-2 (“IL-2”), interleukin-5 (“IL-5”), interleukin-6 (“IL-6”), interleukin-7 (“IL-7”), interleukin 9 (“IL-9”), interleukin-10 (“IL-10”), interleukin-12 (“IL-12”), interleukin-15 (“IL-15”), interleukin-23 (“IL-23”), granulocyte macrophage colony stimulating factor (“GM-CSF”), and granulocyte colony stimulating factor (“G-CSF”)), or a growth factor (e.g., growth hormone (“GH”)), or a coagulation agent (e.g., calcium, vitamin K, tissue factors, such as but not limited to, Hageman factor (factor XII), high-molecular-weight kininogen (HMWK), prekallikrein (PK), coagulation proteins-factors II (prothrombin), factor V, XIIa, VIII, XIIIa, XI, XIa, IX, IXa, X, phospholipid, and fibrin monomer).

Provided herein are antibodies recombinantly fused or chemically conjugated (covalent or non-covalent conjugations) to a heterologous protein or polypeptide (or fragment thereof, preferably to a polypeptide of about 10, about 20, about 30, about 40, about 50, about 60, about 70, about 80, about 90 or about 100 amino acids) to generate fusion proteins. In particular, provided herein are fusion proteins comprising an antigen-binding fragment of an antibody described herein (e.g., a Fab fragment, Fd fragment, Fv fragment, F(ab)₂ fragment, a VH domain, a VH CDR, a VL domain or a VL CDR) and a heterologous protein, polypeptide, or peptide. In one embodiment, the heterologous protein, polypeptide, or peptide that the antibody is fused to is useful for targeting the antibody to a particular cell type, such as a cell that expresses KIT. For example, an antibody that immunospecifically binds to a cell surface receptor expressed by a particular cell type (e.g., an immune cell) can be fused or conjugated to a modified antibody described herein. In specific embodiments, the heterologous protein or polypeptide (or fragment thereof) binds to a second target (e.g., a target other than KIT) (see, e.g., PCT International Patent Application Publication No. WO 2009/088805 and U.S. Patent Application Publication No. US 2009/0148905).

Provided herein is a conjugated or fusion protein comprising any antibody described herein and a heterologous polypeptide (e.g., a polypeptide other than KIT). In one embodiment, a conjugated or fusion protein described herein comprises an anti-KIT antibody described herein, and a heterologous polypeptide. In another embodiment, a conjugated or fusion protein provided herein comprises an antigen-binding fragment of an anti-KIT antibody described herein, and a heterologous polypeptide. In another embodiment, a conjugated or fusion protein described herein comprises a VH domain having the amino acid sequence of any one of the VH domains of an anti-KIT antibody described herein, and/or a VL domain having the amino acid sequence of any one of the VL domains of an anti-KIT antibody described herein, and a heterologous polypeptide. In another embodiment, a conjugated or fusion protein described herein comprises one or more VH CDRs having the amino acid sequence of any one of the VH CDRs of an anti-KIT antibody described herein (e.g., VH CDRs in Tables 3A, 3B, and 3C), and a heterologous polypeptide. In another embodiment, a conjugated or fusion protein comprises one or more VL CDRs having the amino acid sequence of any one of the VL CDRs of an anti-KIT antibody described herein (e.g., VL CDRs in Tables 1A, 1B and 1C), and a heterologous polypeptide. In another embodiment, a conjugated or fusion protein described herein comprises at least one VH domain and at least one VL domain of an anti-KIT antibody described herein, and a heterologous polypeptide. In yet another embodiment, a conjugated or fusion protein described herein comprises at least one VH CDR and at least one VL CDR of an anti-KIT antibody described herein (e.g., VL CDRs in Tables 1A, 1B and 1C and VH CDRs in Tables 3A, 3B and 3C), and a heterologous polypeptide.

In addition, an antibody described herein can be conjugated to therapeutic moieties such as a radioactive metal ion, such as alpha-emitters such as ²¹³Bi or macrocyclic chelators useful for conjugating radiometal ions, including but not limited to, 131In, ¹³¹Lu, ¹³¹Y, ¹³¹Ho, ¹³¹Sm, to polypeptides. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties. In particular embodiments, a linker is an enzyme-cleavable linker or a disulfide linker. In a specific embodiment, the cleavable linker is cleavable via an enzyme such as an aminopeptidase, an aminoesterase, a dipeptidyl carboxy peptidase, or a protease of the blood clotting cascade. In particular embodiments, a linker comprises 1, 2, 3, 4, 5, 6, 7, 8, 9, 10, 11, 12, 13, 14, 15, or 20 amino acid residues. In certain embodiments, a linker consists of 1 to 10 amino acid residues, 1 to 15 amino acid residues, 5 to 20 amino acid residues, 10 to 25 amino acid residues, 10 to 30 amino acid residues, or 10 to 50 amino acid residues.

Moreover, antibodies described herein can be fused to marker sequences, such as a peptide to facilitate purification. In preferred embodiments, the marker amino acid sequence is a hexa-histidine peptide, such as the tag provided in a pQE vector (QIAGEN, Inc.), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “FLAG” tag.

Methods for fusing or conjugating therapeutic moieties (including polypeptides) to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), Thorpe et al., 1982, Immunol. Rev. 62:119-58; U.S. Pat. Nos. 5,336,603, 5,622,929, 5,359,046, 5,349,053, 5,447,851, 5,723,125, 5,783,181, 5,908,626, 5,844,095, and 5,112,946; EP 307,434; EP 367,166; EP 394,827; PCT publications WO 91/06570, WO 96/04388, WO 96/22024, WO 97/34631, and WO 99/04813; Ashkenazi et al., Proc. Natl. Acad. Sci. USA, 88: 10535-10539, 1991; Traunecker et al., Nature, 331:84-86, 1988; Zheng et al., J. Immunol., 154:5590-5600, 1995; Vil et al., Proc. Natl. Acad. Sci. USA, 89:11337-11341, 1992, which are incorporated herein by reference in their entireties.

Fusion proteins can be generated, for example, through the techniques of gene-shuffling, motif-shuffling, exon-shuffling, and/or codon-shuffling (collectively referred to as “DNA shuffling”). DNA shuffling can be employed to alter the activities of antibodies described herein (e.g., antibodies with higher affinities and lower dissociation rates). See, generally, U.S. Pat. Nos. 5,605,793, 5,811,238, 5,830,721, 5,834,252, and 5,837,458; Patten et al., 1997, Cum Opinion Biotechnol. 8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-313 (each of these patents and publications are hereby incorporated by reference in its entirety). Antibodies, or the encoded antibodies, can be altered by being subjected to random mutagenesis by error-prone PCR, random nucleotide insertion or other methods prior to recombination. A polynucleotide encoding an antibody described herein can be recombined with one or more components, motifs, sections, parts, domains, fragments, etc. of one or more heterologous molecules.

An antibody described herein can also be conjugated to a second antibody to form an antibody heteroconjugate as described in U.S. Pat. No. 4,676,980, which is incorporated herein by reference in its entirety.

The therapeutic moiety or drug conjugated or recombinantly fused to an antibody described herein that immunospecifically binds to a KIT antigen can be chosen to achieve the desired prophylactic or therapeutic effect(s). In certain embodiments, the antibody is a modified antibody. A clinician or other medical personnel should consider the following when deciding on which therapeutic moiety or drug to conjugate or recombinantly fuse to an antibody described herein: the nature of the disease, the severity of the disease, and the condition of the subject.

Antibodies described herein can also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### 5.2 Polynucleotides

In certain aspects, provided herein are polynucleotides comprising a nucleotide sequence encoding an antibody described herein or a fragment thereof that immunospecifically binds to a KIT antigen, and vectors comprising such polynucleotides for recombinant expression in host cells (e.g., E. coli and mammalian cells). Provided herein are polynucleotides comprising nucleotide sequences encoding any of the antibodies provided herein, as well as vectors comprising such polynucleotide sequences, e.g., expression vectors for their efficient expression in host cells, e.g., mammalian cells.

In particular aspects, provided herein are polynucleotides comprising nucleotide sequences encoding antibodies (e.g., a human antibody), which immunospecifically bind to a KIT polypeptide (e.g., the D4/D5 region of KIT, for example, human KIT) and comprises an amino acid sequence as described herein, as well as antibodies which compete with such antibodies for binding to a KIT polypeptide (e.g., in a dose-dependent manner), or which binds to the same epitope as that of such antibodies.

In certain aspects, provided herein are polynucleotides comprising a nucleotide sequence encoding the light chain or heavy chain of an antibody described herein. The polynucleotides can comprise nucleotide sequences encoding a light chain comprising the VL FRs and CDRs of antibodies described herein (see, e.g., Tables 1A, 1B, 1C, 2A and 2B). The polynucleotides can comprise nucleotide sequences encoding a heavy chain comprising the VH FRs and CDRs of antibodies described herein (see, e.g., Tables 3A, 3B, 3C, 4A and 4B). In specific embodiments, a polynucleotide described herein encodies a VL chain region having the amino acid sequence of any one of SEQ ID NOs: 176-187, 312-319, 328, and 350. In specific embodiments, a polynucleotide described herein encodies a VH chain region having the amino acid sequence of any one of SEQ ID NOs: 188-199, 320-327, and 329. In particular embodiments, a polynucleotide described herein encodies a VL chain region having the amino acid sequence of any one of SEQ ID NO: 176, 350 or 328. In particular embodiments, a polynucleotide described herein encodies a VH chain region having the amino acid sequence of any one of SEQ ID NO: 188 or 329. In a specific embodiment, a polynucleotide described herein encodies a VL chain region having the amino acid sequence of SEQ ID NO: 176 beginning at the second amino acid residue of SEQ ID NO: 176. In a specific embodiment, a polynucleotide described herein encodies a VL chain region having the amino acid sequence of SEQ ID NO: 350.

In particular embodiments, provided herein are polynucleotides comprising a nucleotide sequence encoding an anti-KIT antibody comprising a VL chain region (e.g., containing FR1-CDR1-FR2-CDR2-FR3-CDR3-FR4) having the amino acid sequences described herein (e.g., see Tables 1A, 1B, 1C, 2A, and 2B). In specific embodiments, provided herein are polynucleotides comprising a nucleotide sequence encoding an anti-KIT antibody comprising a VH chain region (e.g., containing FR1-CDR1-FR2-CDR2-FR3-CDR3-FR4) having the amino acid sequence described herein (e.g., see Tables 3A, 3B, 3C, 4A, and 4B).

In a particular embodiment, a polynucleotide described herein comprises a nucleotide sequence encoding an antibody provided herein comprising a VL chain region having the amino acid sequence of SEQ ID NO: 176 or 350 (VL domain of Ab1), and a VH chain region having the amino acid sequence of SEQ ID NO: 188 (VH domain of Ab1). In a particular embodiment, a polynucleotide described herein comprises a nucleotide sequence encoding an antibody provided herein comprising a VL chain region having the amino acid sequence of SEQ ID NO: 176 (VL domain of Ab1) starting with the second amino acid residue of SEQ ID NO: 176, and a VH chain region having the amino acid sequence of SEQ ID NO: 188 (VH domain of Ab1). In a particular embodiment, a polynucleotide described herein comprises a nucleotide sequence encoding an antibody provided herein comprising a VL chain region having the amino acid sequence of SEQ ID NO: 359 (VL domain of Ab1), and a VH chain region having the amino acid sequence of SEQ ID NO: 188 (VH domain of Ab1). In a particular embodiment, a polynucleotide described herein comprises a nucleotide sequence encoding an antibody provided herein comprising a VL chain region having the amino acid sequence of SEQ ID NO: 177 (VL domain of Ab2), and a VH chain region having the amino acid sequence of SEQ ID NO: 189 (VH domain of Ab2). In a particular embodiment, a polynucleotide described herein comprises a nucleotide sequence encoding an antibody provided herein comprising a VL chain region having the amino acid sequence of SEQ ID NO: 178 (VL domain of Ab3), and a VH chain region having the amino acid sequence of SEQ ID NO: 190 (VH domain of Ab3). In a particular embodiment, a polynucleotide described herein comprises a nucleotide sequence encoding an antibody provided herein comprising a VL chain region having the amino acid sequence of SEQ ID NO: 179 (VL domain of Ab4), and a VH chain region having the amino acid sequence of SEQ ID NO: 191 (VH domain of Ab4). In a particular embodiment, a polynucleotide described herein comprises a nucleotide sequence encoding an antibody provided herein comprising a VL chain region having the amino acid sequence of SEQ ID NO: 180 (VL domain of Ab5), and a VH chain region having the amino acid sequence of SEQ ID NO: 192 (VH domain of Ab5). In a particular embodiment, a polynucleotide described herein comprises a nucleotide sequence encoding an antibody provided herein comprising a VL chain region having the amino acid sequence of SEQ ID NO: 181 (VL domain of Ab6), and a VH chain region having the amino acid sequence of SEQ ID NO: 193 (VH domain of Ab6). In a particular embodiment, a polynucleotide described herein comprises a nucleotide sequence encoding an antibody provided herein comprising a VL chain region having the amino acid sequence of SEQ ID NO: 182 (VL domain of Ab7), and a VH chain region having the amino acid sequence of SEQ ID NO: 194 (VH domain of Ab7). In a particular embodiment, a polynucleotide described herein comprises a nucleotide sequence encoding an antibody provided herein comprising a VL chain region having the amino acid sequence of SEQ ID NO: 183 (VL domain of Ab8), and a VH chain region having the amino acid sequence of SEQ ID NO: 195 (VH domain of Ab8). In a particular embodiment, a polynucleotide described herein comprises a nucleotide sequence encoding an antibody provided herein comprising a VL chain region having the amino acid sequence of SEQ ID NO: 184 (VL domain of Ab9), and a VH chain region having the amino acid sequence of SEQ ID NO: 196 (VH domain of Ab9). In a particular embodiment, a polynucleotide described herein comprises a nucleotide sequence encoding an antibody provided herein comprising a VL chain region having the amino acid sequence of SEQ ID NO: 185 (VL domain of Ab10), and a VH chain region having the amino acid sequence of SEQ ID NO: 197 (VH domain of Ab10). In a particular embodiment, a polynucleotide described herein comprises a nucleotide sequence encoding an antibody provided herein comprising a VL chain region having the amino acid sequence of SEQ ID NO: 186 (VL domain of Ab11), and comprises a VH chain region having the amino acid sequence of SEQ ID NO: 198 (VH domain of Ab11). In a particular embodiment, a polynucleotide described herein comprises a nucleotide sequence encoding an antibody provided herein comprising a VL chain region having the amino acid sequence of SEQ ID NO: 187 (VL domain of Ab12), and a VH chain region having the amino acid sequence of SEQ ID NO: 199 (VH domain of Ab12). In a particular embodiment, a polynucleotide described herein comprises a nucleotide sequence encoding an antibody provided herein comprising a VL chain region having the amino acid sequence of SEQ ID NO: 328 (VL domain of Ab21), and a VH chain region having the amino acid sequence of SEQ ID NO: 329 (VH domain of Ab21).

In a particular embodiment, a polynucleotide described herein comprises a nucleotide sequence encoding an antibody provided herein comprising a VL chain region having the amino acid sequence of any one of SEQ ID NOs: 312-319, and a VH chain region having the amino acid sequence of any one of SEQ ID NOs: 320-327.

In certain embodiments, a polynucleotide described herein comprises a nucleotide sequence encoding an antibody provided herein comprising a variable light (VL) chain region comprising an amino acid described herein (e.g., see FIG. 2), wherein the antibody immunospecifically binds to a KIT polypeptide, e.g., a human KIT polypeptide, for example, a D4/D5 region of KIT (e.g., human KIT), for example SEQ ID NO: 171.

In certain embodiments, a polynucleotide described herein comprises a nucleotide sequence encoding an antibody provided herein comprising a variable heavy (VH) chain region comprising an amino acid sequence described herein (e.g., see FIG. 3), wherein the antibody immunospecifically binds to a KIT polypeptide, e.g., a human KIT polypeptide, for example, a D4/D5 region of KIT (e.g., human KIT), for example SEQ ID NO: 171.

In certain embodiments, a polynucleotide described herein comprises a nucleotide sequence encoding an antibody provided herein comprises one or more VL CDRs having the amino acid sequence described herein (e.g., see Tables 1A, 1B and 1C), wherein the antibody immunospecifically binds to a KIT polypeptide, e.g., a human KIT polypeptide, for example, a D4/D5 region of KIT (e.g., human KIT), for example SEQ ID NO: 171.

In certain embodiments, a polynucleotide described herein comprises a nucleotide sequence encoding an antibody provided herein comprising one or more VH CDRs having the amino acid sequence described herein (e.g., see Tables 3A, 3B and 3C), wherein the antibody immunospecifically binds to a KIT polypeptide, e.g., a human KIT polypeptide, for example, a D4/D5 region of KIT (e.g., human KIT), for example SEQ ID NO: 171.

In another specific embodiment, a polynucleotide described herein comprises a nucleotide sequence encoding an antibody provided herein (e.g., human antibody) comprising: (i) a VL chain region comprising a VL chain region comprising a VL CDR1, VL CDR2, and VL CDR3 having the amino acid sequences of SEQ ID NO: 2 (VL CDR1 of Ab1), SEQ ID NO: 14 (VL CDR2 of Ab1), and SEQ ID NO: 26 (VL CDR3 of Ab1), respectively; and (ii) a VH chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively.

In another specific embodiment, a polynucleotide described herein comprises a nucleotide sequence encoding an antibody provided herein (e.g., human antibody) comprising: (i) a VL chain region comprising a VL chain region comprising a VL CDR1, VL CDR2, and VL CDR3 having the amino acid sequences of SEQ ID NO: 3 (VL CDR1 of Ab2), SEQ ID NO: 15 (VL CDR2 of Ab2), and SEQ ID NO: 27 (VL CDR3 of Ab2), respectively; and (ii) a VH chain region comprising a VH CDR1, VH CDR2, and VH CDR3 having the amino acid sequences of SEQ ID NO: 87 (VH CDR1 of Ab2), SEQ ID NO: 99 (VH CDR2 of Ab2), and SEQ ID NO: 111 (VH CDR3 of Ab2), respectively.

In another specific embodiment, a polynucleotide described herein comprises a nucleotide sequence encoding an antibody provided herein (e.g., human antibody) comprising: (i) a VL chain region comprising a VL chain region comprising a VL CDR1, VL CDR2, and VL CDR3 having the amino acid sequences of SEQ ID NO: 4 (VL CDR1 of Ab3), SEQ ID NO: 16 (VL CDR2 of Ab3), and SEQ ID NO: 28 (VL CDR3 of Ab3), respectively; and (ii) a VH chain region comprising a VH CDR1, VH CDR2, and VH CDR3 having the amino acid sequences of SEQ ID NO: 88 (VH CDR1 of Ab3), SEQ ID NO: 100 (VH CDR2 of Ab3), and SEQ ID NO: 112 (VH CDR3 of Ab3), respectively.

In another specific embodiment, a polynucleotide described herein comprises a nucleotide sequence encoding an antibody provided herein (e.g., human antibody) comprising: (i) a VL chain region comprising a VL CDR1, VL CDR2, and VL CDR3 having the amino acid sequences of SEQ ID NO: 5 (VL CDR1 of Ab4), SEQ ID NO: 17 (VL CDR2 of Ab4), and SEQ ID NO: 29 (VL CDR3 of Ab4), respectively; and (ii) a VH chain region comprising a VH CDR1, VH CDR2, and VH CDR3 having the amino acid sequences of SEQ ID NO: 89 (VH CDR1 of Ab4), SEQ ID NO: 101 (VH CDR2 of Ab4), and SEQ ID NO: 113 (VH CDR3 of Ab4), respectively.

In another specific embodiment, a polynucleotide described herein comprises a nucleotide sequence encoding an antibody provided herein (e.g., human antibody) comprising: (i) a VL chain region comprising a VL CDR1, VL CDR2, and VL CDR3 having the amino acid sequences of SEQ ID NO: 6 (VL CDR1 of Ab5), SEQ ID NO: 18 (VL CDR2 of Ab5), and SEQ ID NO: 30 (VL CDR3 of Ab5), respectively; and (ii) a VH chain region comprising a VH CDR1, VH CDR2, and VH CDR3 having the amino acid sequences of SEQ ID NO: 90 (VH CDR1 of Ab5), SEQ ID NO: 102 (VH CDR2 of Ab5), and SEQ ID NO: 114 (VH CDR3 of Ab5), respectively.

In another specific embodiment, a polynucleotide described herein comprises a nucleotide sequence encoding an antibody provided herein (e.g., human antibody) comprising: (i) a VL chain region comprising a VL CDR1, VL CDR2, and VL CDR3 having the amino acid sequences of SEQ ID NO: 7 (VL CDR1 of Ab6), SEQ ID NO: 19 (VL CDR2 of Ab6), and SEQ ID NO: 31 (VL CDR3 of Ab6), respectively; and (ii) a VH chain region comprising a VH CDR1, VH CDR2, and VH CDR3 having the amino acid sequences of SEQ ID NO: 91 (VH CDR1 of Ab6), SEQ ID NO: 103 (VH CDR2 of Ab6), and SEQ ID NO: 115 (VH CDR3 of Ab6), respectively.

In another specific embodiment, a polynucleotide described herein comprises a nucleotide sequence encoding an antibody provided herein (e.g., human antibody) comprising: (i) a VL chain region comprising a VL CDR1, VL CDR2, and VL CDR3 having the amino acid sequences of SEQ ID NO: 8 (VL CDR1 of Ab7), SEQ ID NO: 20 (VL CDR2 of Ab7), and SEQ ID NO: 32 (VL CDR3 of Ab7), respectively; and (ii) a VH chain region comprising a VH CDR1, VH CDR2, and VH CDR3 having the amino acid sequences of SEQ ID NO: 92 (VH CDR1 of Ab7), SEQ ID NO: 104 (VH CDR2 of Ab7), and SEQ ID NO: 116 (VH CDR3 of Ab7), respectively.

In another specific embodiment, a polynucleotide described herein comprises a nucleotide sequence encoding an antibody provided herein (e.g., human antibody) comprising: (i) a VL chain region comprising a VL CDR1, VL CDR2, and VL CDR3 having the amino acid sequences of SEQ ID NO: 9 (VL CDR1 of Ab8), SEQ ID NO: 21(VL CDR2 of Ab8), and SEQ ID NO: 33 (VL CDR3 of Ab8), respectively; and (ii) a VH chain region comprising a VH CDR1, VH CDR2, and VH CDR3 having the amino acid sequences of SEQ ID NO: 93 (VH CDR1 of Ab8), SEQ ID NO: 105 (VH CDR2 of Ab8), and SEQ ID NO: 117 (VH CDR3 of Ab8), respectively.

In another specific embodiment, a polynucleotide described herein comprises a nucleotide sequence encoding an antibody provided herein (e.g., human antibody) comprising: (i) a VL chain region comprising a VL CDR1, VL CDR2, and VL CDR3 having the amino acid sequences of SEQ ID NO: 10 (VL CDR1 of Ab9), SEQ ID NO: 22 (VL CDR2 of Ab9), and SEQ ID NO: 34 (VL CDR3 of Ab9), respectively; and (ii) a VH chain region comprising a VH CDR1, VH CDR2, and VH CDR3 having the amino acid sequences of SEQ ID NO: 94 (VH CDR1 of Ab9), SEQ ID NO: 106 (VH CDR2 of Ab9), and SEQ ID NO: 118 (VH CDR3 of Ab9), respectively.

In another specific embodiment, a polynucleotide described herein comprises a nucleotide sequence encoding an antibody provided herein (e.g., human antibody) comprising: (i) a VL chain region comprising a VL CDR1, VL CDR2, and VL CDR3 having the amino acid sequences of SEQ ID NO: 11 (VL CDR1 of Ab10), SEQ ID NO: 23 (VL CDR2 of Ab10), and SEQ ID NO: 35 (VL CDR3 of Ab10), respectively; and (ii) a VH chain region comprising a VH CDR1, VH CDR2, and VH CDR3 having the amino acid sequences of SEQ ID NO: 95 (VH CDR1 of Ab10), SEQ ID NO: 107 (VH CDR2 of Ab10), and SEQ ID NO: 119 (VH CDR3 of Ab10), respectively.

In another specific embodiment, a polynucleotide described herein comprises a nucleotide sequence encoding an antibody provided herein (e.g., human antibody) comprising: (i) a VL chain region comprising a VL CDR1, VL CDR2, and VL CDR3 having the amino acid sequences of SEQ ID NO: 12 (VL CDR1 of Ab11), SEQ ID NO: 24 (VL CDR2 of Ab11), and SEQ ID NO: 36 (VL CDR3 of Ab11), respectively; and (ii) a VH chain region comprising a VH CDR1, VH CDR2, and VH CDR3 having the amino acid sequences of SEQ ID NO: 96 (VH CDR1 of Ab11), SEQ ID NO: 108 (VH CDR2 of Ab11), and SEQ ID NO: 120 (VH CDR3 of Ab11), respectively.

In another specific embodiment, a polynucleotide described herein comprises a nucleotide sequence encoding an antibody provided herein (e.g., human antibody) comprising: (i) a VL chain region comprising a VL CDR1, VL CDR2, and VL CDR3 having the amino acid sequences of SEQ ID NO: 13 (VL CDR1 of Ab12), SEQ ID NO: 25 (VL CDR2 of Ab12), and SEQ ID NO: 37 (VL CDR3 of Ab12), respectively; and (ii) a VH chain region comprising a VH CDR1, VH CDR2, and VH CDR3 having the amino acid sequences of SEQ ID NO: 97 (VH CDR1 of Ab12), SEQ ID NO: 109 (VH CDR2 of Ab12), and SEQ ID NO: 121 (VH CDR3 of Ab12), respectively.

In another specific embodiment, a polynucleotide described herein comprises a nucleotide sequence encoding an antibody provided herein (e.g., human antibody) comprising: (i) a VL chain region comprising a VL chain region comprising a VL CDR1, VL CDR2, and VL CDR3 having the amino acid sequences of SEQ ID NO: 330 (VL CDR1 of Ab21), SEQ ID NO: 331 (VL CDR2 of Ab21), and SEQ ID NO: 332 (VL CDR3 of Ab21), respectively; and (ii) a VH chain region comprising a VH CDR1, VH CDR2, and VH CDR3 having the amino acid sequences of SEQ ID NO: 337 (VH CDR1 of Ab21), SEQ ID NO: 338 (VH CDR2 of Ab21), and SEQ ID NO: 339(VH CDR3 of Ab21), respectively.

In specific aspects, provided herein is a polynucleotide described herein comprises a nucleotide sequence encoding an antibody provided herein (e.g., human antibody) which competitively blocks (e.g., in a dose dependent manner), antibodies comprising the amino acid sequences described herein from specific binding to a KIT polypeptide (e.g., a D4/D5 region of KIT, for example human KIT), as determined using assays known to one of skill in the art or described herein (e.g., ELISA competitive assays).

In certain aspects, a polynucleotide provided herein comprises a nucleotide sequence encoding an antibody described herein comprising a VL chain region comprising one or more VL FRs having the amino acid sequence described herein (e.g., see Tables 2A and 2B), wherein the antibody immunospecifically binds to a KIT polypeptide, e.g., a human KIT polypeptide, for example, a D4/D5 region of KIT (e.g., human KIT), for example SEQ ID NO: 171. In certain aspects, a polynucleotide provided herein comprises a nucleotide sequence encoding an antibody described herein comprising a VH chain region comprising one or more VH FRs having the amino acid sequence described herein (e.g., see Tables 4A and 4B), wherein the antibody immunospecifically binds to a KIT polypeptide, e.g., a human KIT polypeptide, for example, a D4/D5 region of KIT (e.g., human KIT), for example SEQ ID NO: 171.

In specific embodiments, a polynucleotide provided herein comprises a nucleotide sequence encoding an antibody described herein comprising: framework regions (e.g., framework regions of the VL domain and VH domain) that are human framework regions, wherein the immunospecifically binds to a KIT polypeptide, e.g., a human KIT polypeptide, for example, a D4/D5 region of KIT (e.g., human KIT, for example SEQ ID NO: 171).

In specific aspects, provided herein is a polynucleotide provided herein comprising a nucleotide sequence encoding an antibody comprising a light chain and a heavy chain, e.g., a separate light chain and heavy chain. With respect to the light chain, in a specific embodiment, a polynucleotide provided herein comprises a nucleotide sequence encoding a kappa light chain. In another specific embodiment, a polynucleotide provided herein comprises a nucleotide sequence encoding a lambda light chain. In yet another specific embodiment, a polynucleotide provided herein comprises a nucleotide sequence encoding an antibody described herein comprising a human kappa light chain or a human lambda light chain. In a particular embodiment, a polynucleotide provided herein comprises a nucleotide sequence encoding an antibody described herein, which immunospecifically binds to a KIT polypeptide (e.g., a KIT polypeptide comprising a D4/D5 region of KIT, for example human KIT (e.g., SEQ ID NO: 171)), wherein the antibody comprises a light chain, and wherein the amino acid sequence of the VL chain region can comprise any amino acid sequence described herein (e.g., any one of SEQ ID NOs: 176-187 and 350), and wherein the constant region of the light chain comprises the amino acid sequence of a human kappa light chain constant region. In another particular embodiment, a polynucleotide provided herein comprises a nucleotide sequence encoding an antibody described herein, which immunospecifically binds to a KIT polypeptide (e.g., a KIT polypeptide comprising a KIT polypeptide comprising a D4/D5 region of KIT, for example human KIT (e.g., SEQ ID NO: 171)), and comprises a light chain, wherein the amino acid sequence of the VL chain region can comprises any amino acid sequence described herein (e.g., any one of SEQ ID NOs: 176-187 and 350), and wherein the constant region of the light chain comprises the amino acid sequence of a human lambda light chain constant region. For example, human constant region sequences can be those described in U.S. Pat. No. 5,693,780. In certain embodiments, the constant region of the light chain comprises the amino acid sequence of SEQ ID NO: 344. In particular embodiments, the constant region of the heavy chain comprises the amino acid sequence of SEQ ID NO: 345.

In a particular embodiment, a polynucleotide provided herein comprises a nucleotide sequence encoding an antibody described herein, which immunospecifically binds to a KIT polypeptide (e.g., a KIT polypeptide comprising a KIT polypeptide comprising a D4/D5 region of KIT, for example human KIT (e.g., SEQ ID NO: 171)), wherein the antibody comprises a heavy chain, wherein the amino acid sequence of the VH chain region can comprise any amino acid sequence described herein (e.g., any of SEQ ID NOs: 188-199), and wherein the constant region of the heavy chain comprises the amino acid sequence of a human gamma (γ) heavy chain constant region. For example, human constant region sequences can be any one of those described in U.S. Pat. No. 5,693,780 or Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242.

In yet another specific embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a VL chain region and a VH chain region comprising any amino acid sequences described herein, and wherein the constant regions comprise the amino acid sequences of the constant regions of a human IgG1 (e.g., isotype a, z, or f) or human IgG4. In a particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a VL chain region and a VH chain region comprising any amino acid sequences described herein, and wherein the constant regions comprise the amino acid sequences of the constant region of a human IgG1 (isotype f).

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising a VL CDR1, VL CDR2, and VL CDR3 having the amino acid sequences of SEQ ID NO: 2 (VL CDR1 of Ab1), SEQ ID NO: 14 (VL CDR2 of Ab1), and SEQ ID NO: 26 (VL CDR3 of Ab1), respectively; (ii) the heavy chain comprises a VH chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising a VL CDR1, VL CDR2, and VL CDR3 having the amino acid sequences of SEQ ID NO: 3 (VL CDR1 of Ab2), SEQ ID NO: 15 (VL CDR2 of Ab2), and SEQ ID NO: 27 (VL CDR3 of Ab2), respectively; (ii) the heavy chain comprises a VH chain region comprising a VH CDR1, VH CDR2, and VH CDR3 having the amino acid sequences of SEQ ID NO: 87 (VH CDR1 of Ab2), SEQ ID NO: 99 (VH CDR2 of Ab2), and SEQ ID NO: 111 (VH CDR3 of Ab2), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising a VL CDR1, VL CDR2, and VL CDR3 having the amino acid sequences of SEQ ID NO: 4 (VL CDR1 of Ab3), SEQ ID NO: 16 (VL CDR2 of Ab3), and SEQ ID NO: 28 (VL CDR3 of Ab3), respectively; (ii) the heavy chain comprises a VH chain region comprising a VH CDR1, VH CDR2, and VH CDR3 having the amino acid sequences of SEQ ID NO: 88 (VH CDR1 of Ab3), SEQ ID NO: 100 (VH CDR2 of Ab3), and SEQ ID NO: 112 (VH CDR3 of Ab3), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising a VL CDR1, VL CDR2, and VL CDR3 having the amino acid sequences of SEQ ID NO: 5 (VL CDR1 of Ab4), SEQ ID NO: 17 (VL CDR2 of Ab4), and SEQ ID NO: 29 (VL CDR3 of Ab4), respectively; (ii) the heavy chain comprises a VH chain region comprising a VH CDR1, VH CDR2, and VH CDR3 having the amino acid sequences of SEQ ID NO: 89 (VH CDR1 of Ab4), SEQ ID NO: 101 (VH CDR2 of Ab4), and SEQ ID NO: 113 (VH CDR3 of Ab4), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising a VL CDR1, VL CDR2, and VL CDR3 having the amino acid sequences of SEQ ID NO: 6 (VL CDR1 of Ab5), SEQ ID NO: 18 (VL CDR2 of Ab5), and SEQ ID NO: 30 (VL CDR3 of Ab5), respectively; (ii) the heavy chain comprises a VH chain region comprising a VH CDR1, VH CDR2, and VH CDR3 having the amino acid sequences of SEQ ID NO: 90 (VH CDR1 of Ab5), SEQ ID NO: 102 (VH CDR2 of Ab5), and SEQ ID NO: 114 (VH CDR3 of Ab5), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising a VL CDR1, VL CDR2, and VL CDR3 having the amino acid sequences of SEQ ID NO: 7 (VL CDR1 of Ab6), SEQ ID NO: 19 (VL CDR2 of Ab6), and SEQ ID NO: 31 (VL CDR3 of Ab6), respectively; (ii) the heavy chain comprises a VH chain region comprising a VH CDR1, VH CDR2, and VH CDR3 having the amino acid sequences of SEQ ID NO: 91 (VH CDR1 of Ab6), SEQ ID NO: 103 (VH CDR2 of Ab6), and SEQ ID NO: 115 (VH CDR3 of Ab6), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising a VL CDR1, VL CDR2, and VL CDR3 having the amino acid sequences of SEQ ID NO: 8 (VL CDR1 of Ab7), SEQ ID NO: 20 (VL CDR2 of Ab7), and SEQ ID NO: 32 (VL CDR3 of Ab7), respectively; (ii) the heavy chain comprises a VH chain region comprising a VH CDR1, VH CDR2, and VH CDR3 having the amino acid sequences of SEQ ID NO: 92 (VH CDR1 of Ab7), SEQ ID NO: 104 (VH CDR2 of Ab7), and SEQ ID NO: 116 (VH CDR3 of Ab7), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising a VL CDR1, VL CDR2, and VL CDR3 having the amino acid sequences of SEQ ID NO: 9 (VL CDR1 of Ab8), SEQ ID NO: 21(VL CDR2 of Ab8), and SEQ ID NO: 33 (VL CDR3 of Ab8), respectively; (ii) the heavy chain comprises a VH chain region comprising a VH CDR1, VH CDR2, and VH CDR3 having the amino acid sequences of SEQ ID NO: 93 (VH CDR1 of Ab8), SEQ ID NO: 105 (VH CDR2 of Ab8), and SEQ ID NO: 117 (VH CDR3 of Ab8), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising a VL CDR1, VL CDR2, and VL CDR3 having the amino acid sequences of SEQ ID NO: 10 (VL CDR1 of Ab9), SEQ ID NO: 22 (VL CDR2 of Ab9), and SEQ ID NO: 34 (VL CDR3 of Ab9), respectively; (ii) the heavy chain comprises a VH chain region comprising a VH CDR1, VH CDR2, and VH CDR3 having the amino acid sequences of SEQ ID NO: 94 (VH CDR1 of Ab9), SEQ ID NO: 106 (VH CDR2 of Ab9), and SEQ ID NO: 118 (VH CDR3 of Ab9), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising a VL CDR1, VL CDR2, and VL CDR3 having the amino acid sequences of SEQ ID NO: 11 (VL CDR1 of Ab10), SEQ ID NO: 23 (VL CDR2 of Ab10), and SEQ ID NO: (VL CDR3 of Ab10), respectively; (ii) the heavy chain comprises a VH chain region comprising a VH CDR1, VH CDR2, and VH CDR3 having the amino acid sequences of SEQ ID NO: 95 (VH CDR1 of Ab10), SEQ ID NO: 107 (VH CDR2 of Ab10), and SEQ ID NO: 119 (VH CDR3 of Ab10), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising a VL CDR1, VL CDR2, and VL CDR3 having the amino acid sequences of SEQ ID NO: 12 (VL CDR1 of Ab11), SEQ ID NO: 24 (VL CDR2 of Ab11), and SEQ ID NO: 36 (VL CDR3 of Ab11), respectively; (ii) the heavy chain comprises a VH chain region comprising a VH CDR1, VH CDR2, and VH CDR3 having the amino acid sequences of SEQ ID NO: 96 (VH CDR1 of Ab11), SEQ ID NO: 108 (VH CDR2 of Ab11), and SEQ ID NO: 120 (VH CDR3 of Ab11), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising a VL CDR1, VL CDR2, and VL CDR3 having the amino acid sequences of SEQ ID NO: 13 (VL CDR1 of Ab12), SEQ ID NO: 25 (VL CDR2 of Ab12), and SEQ ID NO: 37 (VL CDR3 of Ab12), respectively; (ii) the heavy chain comprises a VH chain region comprising a VH CDR1, VH CDR2, and VH CDR3 having the amino acid sequences of SEQ ID NO: 97 (VH CDR1 of Ab12), SEQ ID NO: 109 (VH CDR2 of Ab12), and SEQ ID NO: 121 (VH CDR3 of Ab12), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising a VL CDR1, VL CDR2, and VL CDR3 having the amino acid sequences of SEQ ID NO: 330 (VL CDR1 of Ab21), SEQ ID NO: 331 (VL CDR2 of Ab21), and SEQ ID NO: 332 (VL CDR3 of Ab21), respectively; (ii) the heavy chain comprises a VH chain region comprising a VH CDR1, VH CDR2, and VH CDR3 having the amino acid sequences of SEQ ID NO: 337 (VH CDR1 of Ab21), SEQ ID NO: 338 (VH CDR2 of Ab21), and SEQ ID NO: 339 (VH CDR3 of Ab21), respectively; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain. In specific embodiments, the constant light chain domain comprises the amino acid sequence of SEQ ID NO: 344, and the constant heavy chain domain comprises the amino acid sequence of SEQ ID NO: 345. In specific embodiments, the constant light chain domain comprises the amino acid sequence of SEQ ID NO: 965, and the constant heavy chain domain comprises the amino acid sequence of SEQ ID NO: 345.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising the amino acid sequence of SEQ ID NO: 176 or 350 or 963(VL domain of Ab1); (ii) the heavy chain comprises a VH chain region comprising the amino acid sequence of SEQ ID NO: 188 (VH domain of Ab1); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising the amino acid sequence of SEQ ID NO: 177 (VL domain of Ab2); (ii) the heavy chain comprises a VH chain region comprising the amino acid sequence of SEQ ID NO: 189 (VH domain of Ab2); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising the amino acid sequence of SEQ ID NO: 178 (VL domain of Ab3); (ii) the heavy chain comprises a VH chain region comprising the amino acid sequence of SEQ ID NO: 190 (VH domain of Ab3); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising the amino acid sequence of SEQ ID NO: 179 (VL domain of Ab4); (ii) the heavy chain comprises a VH chain region comprising the amino acid sequence of SEQ ID NO: 191 (VH domain of Ab4); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising the amino acid sequence of SEQ ID NO: 180 (VL domain of Ab5); (ii) the heavy chain comprises a VH chain region comprising the amino acid sequence of SEQ ID NO: 192 (VH domain of Ab5); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising the amino acid sequence of SEQ ID NO: 181 (VL domain of Ab6); (ii) the heavy chain comprises a VH chain region comprising the amino acid sequence of SEQ ID NO: 193 (VH domain of Ab6); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising the amino acid sequence of SEQ ID NO: 182 (VL domain of Ab7); (ii) the heavy chain comprises a VH chain region comprising the amino acid sequence of SEQ ID NO: 194 (VH domain of Ab7); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising the amino acid sequence of SEQ ID NO: 183 (VL domain of Ab8); (ii) the heavy chain comprises a VH chain region comprising the amino acid sequence of SEQ ID NO: 195 (VH domain of Ab8); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising the amino acid sequence of SEQ ID NO: 184 (VL domain of Ab9); (ii) the heavy chain comprises a VH chain region comprising the amino acid sequence of SEQ ID NO: 196 (VH domain of Ab9); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising the amino acid sequence of SEQ ID NO: 185 (VL domain of Ab10); (ii) the heavy chain comprises a VH chain region comprising the amino acid sequence of SEQ ID NO: 197 (VH domain of Ab10); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising the amino acid sequence of SEQ ID NO: 186 (VL domain of Ab11); (ii) the heavy chain comprises a VH chain region comprising the amino acid sequence of SEQ ID NO: 198 (VH domain of Ab11); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising the amino acid sequence of SEQ ID NO: 187 (VL domain of Ab12); (ii) the heavy chain comprises a VH chain region comprising the amino acid sequence of SEQ ID NO: 199 (VH domain of Ab12); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In another particular embodiment, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising the amino acid sequence of SEQ ID NO: 328 or 964 (VL domain of Ab21); (ii) the heavy chain comprises a VH chain region comprising the amino acid sequence of SEQ ID NO: 329 (VH domain of Ab21); (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In certain embodiments, with respect to a polynucleotide provided herein comprising a nucleotide sequence encoding a VL chain region and VH chain region of any of these antibodies described herein, the polynucleotide of the VL chain region further comprises human framework regions; and the VH chain region further comprises human framework regions.

In certain embodiments, a polynucleotide provided herein comprises a nucleotide sequence encoding an antibody described herein, which immunospecifically binds to a KIT polypeptide (e.g., a D4/D5 region of KIT, for example human KIT), wherein the antibody comprises a light chain and a heavy chain, and wherein (i) the light chain comprises a VL chain region comprising human framework regions; (ii) the heavy chain comprises a VH chain region comprising human framework regions; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 (optionally IgG1 (isotype f)) heavy chain.

In a specific embodiment, provided herein are polynucleotides comprising a nucleotide sequence encoding an anti-KIT antibody, or a fragment or domain thereof, designated herein (see, e.g., Tables 1A-4B and FIGS. 5A-5W) as Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab24-Ab36, or Ab38-Ab192. In a specific embodiment, provided herein are polynucleotides comprising a nucleotide sequence encoding an anti-KIT antibody, or a fragment or domain thereof, designated herein (see, e.g., Tables 1A-4B and FIGS. 5A-5W) as Ab1. In a specific embodiment, provided herein are polynucleotides comprising a nucleotide sequence encoding an anti-KIT antibody, or a fragment or domain thereof, designated herein (see, e.g., Tables 1A-4B and FIGS. 5A-5W) as Ab21.

In certain embodiments, polynucleotides described herein comprise a nucleotide sequence encoding a light chain or a VL chain region comprising the VL CDR1, VL CDR2, and VL CDR3 amino acid sequences of any one of antibodies: Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab24-Ab36, and Ab38-Ab192 (e.g., as designated in Tables 1A, 1B, 1C). In certain embodiments, polynucleotides described herein comprise a nucleotide sequence encoding a light chain or a VL chain region comprising the VL CDR1, VL CDR2, and VL CDR3 amino acid sequences of those of the Ab1 antibody (e.g., as designated in Tables 1A, 1B, 1C). In certain embodiments, polynucleotides described herein comprise a nucleotide sequence encoding a VL chain region comprising the amino acid sequence of the Ab1 antibody as designated in Tables 1A, 1B, 1C, 2A and 2B, wherein the VL FR1 comprises the amino acid sequence beginning at the second amino acid residue of the VL FR1 sequence depicted in Table 2A and 2B. In certain embodiments, polynucleotides described herein comprise a nucleotide sequence encoding a light chain or a VL chain region comprising the VL CDR1, VL CDR2, and VL CDR3 amino acid sequences of those of the Ab21 antibody (e.g., as designated in Tables 1A, 1B, 1C, 2A and 2B). In certain embodiments, polynucleotides described herein comprise a nucleotide sequence encoding a heavy chain or a VH chain region comprising the VH CDR1, VH CDR2, and VH CDR3 amino acid sequences of any one of the antibodies: Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, Ab21, Ab24-Ab36, and Ab38-Ab192 (e.g., as designated in Tables 3A, 3B, 3C, 4A and 4B). In certain embodiments, polynucleotides described herein comprise a nucleotide sequence encoding a heavy chain or a VH chain region comprising the VH CDR1, VH CDR2, and VH CDR3 amino acid sequences of those of the Ab1 antibody (e.g., as designated in Tables 3A, 3B, 3C, 4A and 4B). In certain embodiments, polynucleotides described herein comprise a nucleotide sequence encoding a heavy chain or a VH chain region comprising the VH CDR1, VH CDR2, and VH CDR3 amino acid sequences of those of the Ab21 antibody (e.g., as designated in Tables 3A, 3B, 3C, 4A and 4B). In certain embodiments, polynucleotides described herein comprise a nucleotide sequence encoding a light chain or a VL chain region comprising the VL FR1, VL FR2, VL FR3, and VL FR4 amino acid sequences of those of the Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, or Ab21 antibody (e.g., as designated in Tables 1A, 1B, 2A, and 2B). In certain embodiments, polynucleotides described herein comprise a nucleotide sequence encoding a light chain or a VL chain region comprising the VL FR1, VL FR2, VL FR3, and VL FR4 amino acid sequences of those of the Ab1 antibody (e.g., as designated in Tables 1A, 1B, 2A, and 2B). In certain embodiments, polynucleotides described herein comprise a nucleotide sequence encoding a light chain or a VL chain region comprising the VL FR1, VL FR2, VL FR3, and VL FR4 amino acid sequences of those of the Ab1 antibody (e.g., as designated in Tables 1A, 1B, 2A, and 2B), wherein the VL FR1 comprising the amino acid sequence beginning at the second amino acid residue of the VL FR1 sequences depicted in Table 2A or 2B. In certain embodiments, polynucleotides described herein comprise a nucleotide sequence encoding a light chain or a VL chain region comprising the VL FR1, VL FR2, VL FR3, and VL FR4 amino acid sequences of those of the Ab21 antibody (e.g., as designated in Tables 1A, 1B, 2A, and 2B). In certain embodiments, polynucleotides described herein comprise a nucleotide sequence encoding a heavy chain or a VH chain region comprising the VH FR1, VH FR2, VH FR3, and VH FR4 amino acid sequences of those of the Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, Ab20, or Ab21 antibody (e.g., as designated in Tables 3A, 3B, 4A and 4B). In certain embodiments, polynucleotides described herein comprise a nucleotide sequence encoding a heavy chain or a VH chain region comprising the VH FR1, VH FR2, VH FR3, and VH FR4 amino acid sequences of those of the Ab1 antibody (e.g., as designated in Tables 3A, 3B, 4A and 4B). In certain embodiments, polynucleotides described herein comprise a nucleotide sequence encoding a heavy chain or a VH chain region comprising the VH FR1, VH FR2, VH FR3, and VH FR4 amino acid sequences of those of the Ab21 antibody (e.g., as designated in Tables 3A, 3B, 4A and 4B).

Also provided herein are polynucleotides encoding an anti-KIT antibody or a fragment thereof that are optimized, e.g., by codon/RNA optimization, replacement with heterologous signal sequences, and elimination of mRNA instability elements. Methods to generate optimized nucleic acids encoding an anti-KIT antibody or a fragment thereof (e.g., light chain, heavy chain, VH domain, or VL domain) for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In some embodiments, it can be desirable to alter one or more codons to encode a conservative mutation, e.g., a similar amino acid with similar chemical structure and properties and/or function as the original amino acid. Such methods can increase expression of an anti-KIT antibody or fragment thereof by at least 1 fold, 2 fold, 3 fold, 4 fold, 5 fold, 10 fold, 20 fold, 30 fold, 40 fold, 50 fold, 60 fold, 70 fold, 80 fold, 90 fold, or 100 fold or more relative to the expression of an anti-KIT antibody encoded by polynucleotides that have not been optimized.

An optimized polynucleotide sequence encoding an anti-KIT antibody described herein or a fragment thereof can hybridize to an unoptimized polynucleotide sequence encoding an anti-KIT antibody described herein or a fragment thereof. In specific embodiments, an optimized nucleotide sequence encoding an anti-KIT antibody described herein or a fragment hybridizes under high stringency conditions to an unoptimized polynucleotide sequence encoding an anti-KIT antibody described herein or a fragment thereof. In a specific embodiment, an optimized nucleotide sequence encoding an anti-KIT antibody described herein or a fragment thereof hybridizes under high stringency, intermediate or lower stringency hybridization conditions to an unoptimized nucleotide sequence encoding an anti-KIT antibody described herein or a fragment thereof. Information regarding hybridization conditions have been described, see, e.g., U.S. Patent Application Publication No. US 2005/0048549 (e.g., paragraphs 72-73).

The polynucleotides can be obtained, and the nucleotide sequence of the polynucleotides determined, by any method known in the art. Nucleotide sequences encoding antibodies described herein, e.g., antibodies described in Tables 2A-4B, and modified versions of these antibodies can be determined using methods well known in the art, i.e., nucleotide codons known to encode particular amino acids are assembled in such a way to generate a nucleic acid that encodes the antibody. Such a polynucleotide encoding the antibody can be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody described herein can be generated from nucleic acid from a suitable source (e.g., a hybridoma) using methods well known in the art (e.g., PCR and other molecular cloning methods). If a clone containing a nucleic acid encoding a particular antibody is not available, but the sequence of the antibody molecule is known, a nucleic acid encoding the immunoglobulin can be chemically synthesized or obtained from a suitable source (e.g., an antibody cDNA library or a cDNA library generated from, or nucleic acid, preferably poly A+ RNA, isolated from, any tissue or cells expressing the antibody, such as hybridoma cells selected to express an antibody described herein) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody Amplified nucleic acids generated by PCR can then be cloned into replicable cloning vectors using any method well known in the art.

DNA encoding anti-KIT antibodies described herein can be readily isolated and sequenced using conventional procedures (e.g., by using oligonucleotide probes that are capable of binding specifically to genes encoding the heavy and light chains of the anti-KIT antibodies). Hybridoma cells can serve as a source of such DNA. Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of anti-KIT antibodies in the recombinant host cells.

In phage display methods, functional antibody domains are displayed on the surface of phage particles which carry the polynucleotide sequences encoding them. In particular, a library of DNA sequences encoding VH and VL domains are generated (e.g., amplified from animal cDNA libraries such as human cDNA libraries or random libraries are generated by chemical synthesis). The DNA encoding the VH and VL domains are recombined together with an scFv linker by PCR and cloned into a phagemid vector. The vector is electroporated in E. coli and the E. coli is infected with helper phage. Phage expressing an antigen-binding domain that binds to a particular antigen can be selected or identified with antigen, e.g., using labeled antigen or antigen bound or captured to a solid surface or bead. After phage selection, the antibody coding regions from the phage can be isolated and used to generate whole antibodies, including human antibodies, or any other desired antigen-binding fragment, and expressed in any desired host, including mammalian cells, insect cells, plant cells, yeast, and bacteria, e.g., as described below. Techniques to recombinantly produced Fab, Fab′ and F(ab′)₂ fragments can also be employed using methods known in the art such as those disclosed in PCT Publication No. WO 92/22324; Mullinax et al., 1992, BioTechniques, 12(6):864-869; Sawai et al., 1995, AJR1, 34:26-34; and Better et al., 1988, Science, 240:1041-1043.

Antibodies can be isolated from antibody phage libraries generated using the techniques described in McCafferty et al., Nature, 348:552-554 (1990). Clackson et al., Nature, 352:624-628 (1991). Marks et al., J. Mol. Biol., 222:581-597 (1991) describe the isolation of murine and human antibodies, respectively, using phage libraries. Chain shuffling can be used in the production of high affinity (nM range) human antibodies (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a heavy chain constant region, e.g., the human gamma 4 constant region, and the PCR amplified VL domains can be cloned into vectors expressing a light chain constant region, e.g., human kappa or lambda constant regions. In certain embodiments, the vectors for expressing the VH or VL domains comprise an EF-1a promoter, a secretion signal, a cloning site for the variable domain, constant domains, and a selection marker such as neomycin. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

In a non-limiting example, the Dyax (Cambridge, Mass.) technology platform can be used to convert Fab-phage or Fabs to complete IgG antibodies, such as the Dyax pR rapid reformatting vectors (RR). Briefly, by PCR, a Fab-encoding DNA fragment is inserted into a Dyax pR-RRV between a eukaryotic leader sequence and an IgG heavy chain constant region cDNA. Antibody expression is driven by the human cytomegalovirus (hCMV). In a second cloning step, bacterial regulatory elements are replaced by the appropriate eukaryotic sequences (i.e., the IRES (internal ribosome entry site) motif). The expression vector can also include the SV40 origin of replication. The Dyax pRh1(a,z), pRh1(f), pRh4 and pR m2a are expression vectors allowing expression of reformatted FAbs as human IgG1 (isotype a,z), human IgG1 (isotype F), human IgG4, and mouse IgG2a, respectively. Expressing vectors can be introduced into a suitable host cell (e.g., HEK293T cells, CHO cells)) for expression and purification.

The DNA also can be modified, for example, by substituting the coding sequence for human heavy and light chain constant domains in place of the homologous murine sequences (U.S. Pat. No. 4,816,567; Morrison et al., Proc. Natl. Acad. Sci. USA, 81:6851 (1984)), or by covalently joining to the immunoglobulin coding sequence all or part of the coding sequence for a non-immunoglobulin polypeptide.

Also provided are polynucleotides that hybridize under high stringency, intermediate or lower stringency hybridization conditions to polynucleotides that encode an antibody described herein. In specific embodiments, polynucleotides described herein hybridize under high stringency, intermediate or lower stringency hybridization conditions to polynucleotides encoding a VH chain region (e.g., any one of SEQ ID NOs: 188-199) and/or VL chain region (e.g., any one of SEQ ID NOs: 176-187, 350, 963, and 964) provided herein. In specific embodiments, polynucleotides described herein hybridize under high stringency, intermediate or lower stringency hybridization conditions to polynucleotides which are complements to polynucleotides encoding a VH chain region (e.g., any one of SEQ ID NOs: 188-199) and/or VL chain region (e.g., any one of SEQ ID NOs: 176-187, 350, 963, and 964) provided herein.

Hybridization conditions have been described in the art and are known to one of skill in the art. For example, hybridization under stringent conditions can involve hybridization to filter-bound DNA in 6× sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions are known to those of skill in the art and have been described, see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3.

### 5.3 Recombinant Expression of Antibodies and Host Cells

In certain aspects, provided herein are host cells recombinantly expressing the antibodies described herein and related expression vectors. Provided herein are expression vectors comprising polynucleotides comprising nucleotide sequences encoding anti-KIT antibodies or a fragment for recombinant expression in host cells, preferably in mammalian cells. Also provided herein are host cells comprising such expression vectors for recombinantly expressing anti-KIT antibodies described herein. In certain aspects, provided herein are host cells engineered to contain nucleic acid(s) encoding an antibody as described herein. In further aspects, provided herein are host cells engineered to express an antibody as described herein.

Recombinant expression of an antibody described herein (e.g., a full-length antibody, heavy and/or light chain of an antibody, or a single chain antibody described herein) that immunospecifically binds to a KIT antigen can involve construction of an expression vector containing a polynucleotide that encodes the antibody. Once a polynucleotide encoding an antibody molecule, heavy and/or light chain of an antibody, or fragment thereof (preferably, but not necessarily, containing the heavy and/or light chain variable domain) described herein has been obtained, the vector for the production of the antibody molecule can be produced by recombinant DNA technology using techniques well-known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding an antibody molecule described herein, a heavy or light chain of an antibody, a heavy or light chain variable domain of an antibody or a fragment thereof, or a heavy or light chain CDR, operably linked to a promoter. Such vectors can include the nucleotide sequence encoding the constant region of the antibody molecule (see, e.g., International Publication Nos. WO 86/05807 and WO 89/01036; and U.S. Pat. No. 5,122,464) and the variable domain of the antibody can be cloned into such a vector for expression of the entire heavy, the entire light chain, or both the entire heavy and light chains.

The expression vector is transferred to a host cell by conventional techniques and the transfected cells are then cultured by conventional techniques to produce an antibody described herein. Thus, provided herein are host cells containing a polynucleotide encoding an antibody described herein or fragments thereof, or a heavy or light chain thereof, or fragment thereof, or a single chain antibody described herein, operably linked to a heterologous promoter. In certain embodiments for the expression of double-chained antibodies, vectors encoding both the heavy and light chains can be co-expressed in the host cell for expression of the entire immunoglobulin molecule, as detailed below.

A variety of host-expression vector systems can be utilized to express antibody molecules described herein (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, MDCK, HEK 293, NSO, PER.C6, VERO, CRL7O3O, HsS78Bst, HeLa, and NIH 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary (CHO) cells, in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In certain embodiments, antibodies described herein are produced by CHO cells or NSO cells. In a specific embodiment, the expression of nucleotide sequences encoding antibodies described herein which immunospecifically bind to a KIT antigen is regulated by a constitutive promoter, inducible promoter or tissue specific promoter.

In bacterial systems, a number of expression vectors can be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruther et al., 1983, EMBO 12:1791), in which the antibody coding sequence can be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the antibody coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals can also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bittner et al., 1987, Methods in Enzymol. 153:51-544).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, COS, MDCK, HEK 293, NIH 3T3, W138, BT483, Hs578T, HTB2, BT2O and T47D, NSO (a murine myeloma cell line that does not endogenously produce any immunoglobulin chains), CRL7O3O and HsS78Bst cells. In preferred embodiments, fully human, monoclonal anti-KIT antibodies described herein are produced in mammalian cells, such as CHO cells.

For long-term, high-yield production of recombinant proteins, stable expression is preferred. For example, cell lines which stably express the antibody molecule can be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express the antibody molecule. Such engineered cell lines can be particularly useful in screening and evaluation of compositions that interact directly or indirectly with the antibody molecule.

A number of selection systems can be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann Rev. Pharmacol. Toxicol. 32:573-596; Mulligan, 1993, Science 260:926-932; and Morgan and Anderson, 1993, Ann Rev. Biochem. 62:191-217; May, 1993, TIB TECH 11(5):155-2 15); and hygro, which confers resistance to hygromycin (Santerre et al., 1984, Gene 30:147). Methods commonly known in the art of recombinant DNA technology can be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds.), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3 (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell can be co-transfected with two or more expression vectors described herein, the first vector encoding a heavy chain derived polypeptide and the second vector encoding a light chain derived polypeptide. The two vectors can contain identical selectable markers which enable equal expression of heavy and light chain polypeptides. The host cells can be co-transfected with different amounts of the two or more expression vectors. For example, host cells can be transfected with any one of the following ratios of a first expression vector and a second expression vector: 1:1, 1:2, 1:3, 1:4, 1:5, 1:6, 1:7, 1:8, 1:9, 1:10, 1:12, 1:15, 1:20, 1:25, 1:30, 1:35, 1:40, 1:45, or 1:50.

Alternatively, a single vector can be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2197-2199). The coding sequences for the heavy and light chains can comprise cDNA or genomic DNA. The expression vector can be monocistronic or multicistronic. A multicistronic nucleic acid construct can encode 2, 3, 4, 5, 6, 7, 8, 9, 10 or more, or in the range of 2-5, 5-10 or 10-20 genes/nucleotide sequences. For example, a bicistronic nucleic acid construct can comprise in the following order a promoter, a first gene (e.g., heavy chain of an antibody described herein), and a second gene and (e.g., light chain of an antibody described herein). In such an expression vector, the transcription of both genes can be driven by the promoter, whereas the translation of the mRNA from the first gene can be by a cap-dependent scanning mechanism and the translation of the mRNA from the second gene can be by a cap-independent mechanism, e.g., by an IRES.

Once an antibody molecule described herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies described herein can be fused to heterologous polypeptide sequences described herein or otherwise known in the art to facilitate purification.

### 5.4 Methods of Producing Antibodies

Antibodies described herein that immunospecifically bind to a KIT antigen can be produced by any method known in the art for the synthesis of antibodies, for example, by chemical synthesis or by recombinant expression techniques. The methods described herein employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Sambrook et al. (2001) Molecular Cloning: A Laboratory Manual, Cold Spring Harbor Laboratory Press, Cold Spring Harbor, N.Y.; Ausubel et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press.

In some embodiments, human antibodies are produced. Human antibodies can be produced using any method known in the art. For example, transgenic mice which are incapable of expressing functional endogenous immunoglobulins, but which can express human immunoglobulin genes, can be used. In particular, he human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of an antigen (e.g., KIT). Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, 1995, Int. Rev. Immunol. 13:65-93. For a detailed discussion of this technology for producing human antibodies and human monoclonal antibodies and protocols for producing such antibodies, see, e.g., PCT publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598. In addition, companies such as Abgenix, Inc. (Freemont, Calif.) and Genpharm (San Jose, Calif.) can be engaged to provide human antibodies directed against a selected antigen using technology similar to that described above.

In some embodiments, human antibodies can be produced using mouse-human hybridomas. For example, human peripheral blood lymphocytes transformed with Epstein-Ban^{-virus (EBV) can be fused with mouse myeloma cells to produce mouse-human hybridomas secreting human monoclonal antibodies, and these mouse-human hybridomas can be screened to determine ones which secrete human monoclonal antibodies that immunospecifically bind to a target antigen (e.g., KIT). Such methods are known and are described in the art, see, e.g., Shinmoto et al., Cytotechnology,} 2004, 46:19-23; Naganawa et al., Human Antibodies, 2005, 14:27-31.

In some embodiments, human antibodies can be generated by inserting human CDRs (e.g., VL CDRs and/or VH CDRs) of an antibody described herein into an expression vector containing nucleotide sequences encoding human framework region sequences. In certain embodiments, such expression vectors further comprise nucleotide sequences encoding a constant region of a light and/or heavy chain. In some embodiments, human antibodies can be generated by inserting human CDRs (e.g., VL CDRs and/or VH CDRs) of an antibody obtained from a phage library into such human expression vectors.

Polyclonal antibodies that immunospecifically bind to an antigen can be produced by various procedures well-known in the art. For example, a human antigen can be administered to various host animals including, but not limited to, sheep, goats, rabbits, mice, rats, etc. to induce the production of sera containing polyclonal antibodies specific for a human antigen. Various adjuvants can be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art. Additionally, a RIMMS (repetitive immunization multiple sites) technique can be used to immunize an animal (Kilptrack et al., 1997 Hybridoma 16:381-9).

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling et al., in: Monoclonal Antibodies and T-Cell Hybridomas 563 681 (Elsevier, N.Y., 1981). The term “monoclonal antibody” as used herein is not limited to antibodies produced through hybridoma technology.

Methods for producing and screening for specific antibodies using hybridoma technology are routine and well known in the art. Briefly, mice can be immunized with an antigen (e.g., KIT, preferably the D4/D5 region of KIT) and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the American Type Culture Collection (ATCC®) (Manassas, Va.), to form hybridomas. Hybridomas are selected and cloned by limited dilution.

The hybridoma clones are then assayed by methods known in the art for cells that secrete antibodies capable of binding a polypeptide of the antigen (e.g., KIT). Ascites fluid, which generally contains high levels of antibodies, can be generated by immunizing mice with positive hybridoma clones.

Accordingly, described herein are methods of generating antibodies by culturing a hybridoma cell secreting an anti-KIT antibody wherein, the hybridoma is generated by fusing splenocytes isolated from a mouse immunized with a KIT antigen with myeloma cells and then screening the hybridomas resulting from the fusion for hybridoma clones that secrete an antibody able to bind to the KIT antigen.

Antibodies described herein include antibody fragments which recognize specific KIT antigens and can be generated by any technique known to those of skill in the art. For example, Fab and F(ab′)₂ fragments described herein can be produced by proteolytic cleavage of immunoglobulin molecules, using enzymes such as papain (to produce Fab fragments) or pepsin (to produce F(ab′)₂ fragments). A Fab fragment corresponds to one of the two identical arms of an antibody molecule and contains the complete light chain paired with the VH and CH1 domains of the heavy chain. A F(ab′)₂ fragment contains the two antigen-binding arms of an antibody molecule linked by disulfide bonds in the hinge region.

Further, the antibodies described herein can also be generated using various phage display methods known in the art. In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods are typically filamentous phage including fd and M13, and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies described herein include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Application No. PCT/GB91/O1 134; International Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce antibody fragments such as Fab, Fab′ and F(ab′)₂ fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043.

In one aspect, to generate whole antibodies, PCR primers including VH or VL nucleotide sequences, a restriction site, and a flanking sequence to protect the restriction site can be used to amplify the VH or VL sequences from a template, e.g., scFv clones. Utilizing cloning techniques known to those of skill in the art, the PCR amplified VH domains can be cloned into vectors expressing a VH constant region, and the PCR amplified VL domains can be cloned into vectors expressing a VL constant region, e.g., human kappa or lambda constant regions. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it can be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741.

A chimeric antibody is a molecule in which different portions of the antibody are derived from different immunoglobulin molecules. For example, a chimeric antibody can contain a variable region of a mouse monoclonal antibody fused to a constant region of a human antibody. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415.

A humanized antibody is capable of binding to a predetermined antigen and which comprises a framework region having substantially the amino acid sequence of a human immunoglobulin and CDRs having substantially the amino acid sequence of a non-human immunoglobulin. A humanized antibody comprises substantially all of at least one, and typically two, variable domains (Fab, Fab′, F(ab′)₂, Fabc, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also can include the CH1, hinge, CH2, CH3, and CH4 regions of the heavy chain. The humanized antibody can be selected from any class of immunoglobulins, including IgM, IgG, IgD, IgA and IgE, and any isotype, including IgG₁, IgG₂, IgG₃ and IgG₄. Usually the constant domain is a complement fixing constant domain where it is desired that the humanized antibody exhibit cytotoxic activity, and the class is typically IgG1. Where such cytotoxic activity is not desirable, the constant domain can be of the IgG₂ class. Examples of VL and VH constant domains that can be used in certain embodiments described herein include, but are not limited to, C-kappa and C-gamma-1 (nG1m) described in Johnson et al. (1997) J. Infect. Dis. 176, 1215-1224 and those described in U.S. Pat. No. 5,824,307. The humanized antibody can comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework can be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental FR and CDR sequences, more often 90%, and most preferably greater than 95%. Humanized antibodies can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. No. 6,407,213, U.S. Pat. No. 5,766,886, WO 9317105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp):5973s-5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1 (Feb. 24, 2005), which is incorporated by reference herein in its entirety. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions (see, e.g., Queen et al., U.S. Pat. No. 5,585,089; and Reichmann et al., 1988, Nature 332:323).

Single domain antibodies, for example, antibodies lacking the light chains, can be produced by methods well-known in the art. See Riechmann et al., 1999, J. Immunol. 231:25-38; Nuttall et al., 2000, Curr. Pharm. Biotechnol. 1(3):253-263; Muylderman, 2001, J. Biotechnol. 74(4):277302; U.S. Pat. No. 6,005,079; and International Publication Nos. WO 94/04678, WO 94/25591, and WO 01/44301.

Further, the antibodies that immunospecifically bind to the KIT antigen can, in turn, be utilized to generate anti-idiotype antibodies that “mimic” an antigen using techniques well known to those skilled in the art. (See, e.g., Greenspan & Bona, 1989, FASEB J. 7(5):437-444; and Nissinoff, 1991, J. Immunol. 147(8):2429-2438).

### 5.5 Pharmaceutical Compositions and Kits

Provided herein are compositions, pharmaceutical compositions, and kits comprising one or more antibodies described herein, e.g., an antibody comprising CDRs of antibody Ab1, or CDRs of antibody Ab21, or an antigen-binding fragment thereof. In particular aspects, compositions described herein can be for in vitro, in vivo, or ex vivo uses.

Therapeutic formulations containing one or more antibodies provided herein can be prepared for storage by mixing the antibody having the desired degree of purity with optional physiologically acceptable carriers, excipients or stabilizers (Remington's Pharmaceutical Sciences (1990) Mack Publishing Co., Easton, Pa.; Remington: The Science and Practice of Pharmacy, 21st ed. (2006) Lippincott Williams & Wilkins, Baltimore, Md.), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Formulations, such as those described herein, can also contain more than one active compounds (for example, molecules, e.g., antibody or antibodies described herein) as necessary for the particular indication being treated. In certain embodiments, formulations comprise an antibody provided herein and one or more active compounds with complementary activities that do not adversely affect each other. Such molecules are suitably present in combination in amounts that are effective for the purpose intended. For example, an antibody described herein can be combined with one or more other therapeutic agents. Such combined therapy can be administered to the patient serially or simultaneously or in sequence.

The formulations to be used for in vivo administration can be sterile. This is readily accomplished by filtration through, e.g., sterile filtration membranes.

The pharmaceutical compositions provided herein contain therapeutically effective amounts of one or more of the antibodies provided herein, and optionally one or more additional prophylactic of therapeutic agents, in a pharmaceutically acceptable carrier. Such pharmaceutical compositions are useful in the prevention, treatment, management or amelioration of a KIT-mediated disorder or disease, such as an inflammatory bowl disease, or one or more of the symptoms thereof.

Pharmaceutical carriers suitable for administration of the compounds provided herein include any such carriers known to those skilled in the art to be suitable for the particular mode of administration.

In addition, the antibodies described herein can be formulated as the sole pharmaceutically active ingredient in the composition or can be combined with other active ingredients (such as one or more other prophylactic or therapeutic agents).

The compositions can contain one or more antibodies provided herein. In one embodiment, the antibodies are formulated into suitable pharmaceutical preparations, such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the antibodies described above are formulated into pharmaceutical compositions using techniques and procedures well known in the art (see, e.g., Ansel (1985) Introduction to Pharmaceutical Dosage Forms, 4^{th} Ed., p. 126).

In the compositions, one or more antibodies provided herein is (are) mixed with a suitable pharmaceutical carrier. The concentrations of the antibody or antibodies in the compositions can, for example, be effective for delivery of an amount, upon administration, that treats, prevents, or ameliorates a KIT-mediated disorder or disease or symptom thereof.

In one embodiment, the compositions are formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

An antibody provided herein is included in the pharmaceutically acceptable carrier in an effective amount sufficient to exert a therapeutically useful effect in the absence of undesirable side effects on the patient treated. The therapeutically effective concentration can be determined empirically by testing the compounds in in vitro and in vivo systems using routine methods and then extrapolated therefrom for dosages for humans.

The concentration of antibody in the pharmaceutical composition will depend on, e.g., the physicochemical characteristics of the antibody, the dosage schedule, and amount administered as well as other factors known to those of skill in the art.

In one embodiment, a therapeutically effective dosage produces a serum concentration of antibody of from about 0.1 ng/ml to about 50-100 μg/ml. The pharmaceutical compositions, in another embodiment, provide a dosage of from about 0.001 mg to about 2000 mg of antibody per kilogram of body weight per day. Pharmaceutical dosage unit forms can be prepared to provide from about 0.01 mg, 0.1 mg or 1 mg to about 500 mg, 1000 mg or 2000 mg, and in one embodiment from about 10 mg to about 500 mg of the antibody and/or a combination of other optional essential ingredients per dosage unit form.

The antibody can be administered at once, or can be divided into a number of smaller doses to be administered at intervals of time. It is understood that the precise dosage and duration of treatment is a function of the disease being treated and can be determined empirically using known testing protocols or by extrapolation from in vivo or in vitro test data. It is to be noted that concentrations and dosage values can also vary with the severity of the condition to be alleviated. It is to be further understood that for any particular subject, specific dosage regimens can be adjusted over time according to the individual need and the professional judgment of the person administering or supervising the administration of the compositions, and that the concentration ranges set forth herein are exemplary only and are not intended to limit the scope or practice of the claimed compositions.

Upon mixing or addition of the antibody, the resulting mixture can be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and can be empirically determined.

The pharmaceutical compositions are provided for administration to humans and animals in unit dosage forms, such as sterile parenteral solutions or suspensions, tablets, capsules, pills, powders, granules, and oral solutions or suspensions, and oil-water emulsions containing suitable quantities of the compounds or pharmaceutically acceptable derivatives thereof. The antibody is, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the antibody sufficient to produce the desired therapeutic effect, in association with the required pharmaceutical carrier, vehicle or diluent. Examples of unit-dose forms include ampoules and syringes and individually packaged tablets or capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

In certain embodiments, one or more anti-KIT antibodies described herein are in a liquid pharmaceutical formulation. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered can also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see, e.g., Remington's Pharmaceutical Sciences (1990) Mack Publishing Co., Easton, Pa.; Remington: The Science and Practice of Pharmacy, 21st ed. (2006) Lippincott Williams & Wilkins, Baltimore, Md.

Dosage forms or compositions containing antibody in the range of 0.005% to 100% with the balance made up from non-toxic carrier can be prepared. Methods for preparation of these compositions are known to those skilled in the art.

Parenteral administration, in one embodiment, is characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The antibody diffuses through the outer polymeric membrane in a release rate controlling step. The amount of antibody contained in such parenteral compositions is highly dependent on the specific nature thereof, as well as the activity of the compound and the needs of the subject.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active molecule (e.g., an antibody or antibodies described herein) is adjusted so that an injection provides an effective amount to produce the desired pharmacological effect. The exact dose depends on the age, weight and condition of the patient or animal as is known in the art.

The unit-dose parenteral preparations can be packaged in an ampoule, a vial or a syringe with a needle. All preparations for parenteral administration can be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound to the treated tissue(s).

The antibody can be suspended in micronized or other suitable form. The form of the resulting mixture depends upon a number of factors, including the intended mode of administration and the solubility of the compound in the selected carrier or vehicle. The effective concentration is sufficient for ameliorating the symptoms of the condition and can be empirically determined.

In other embodiments, the pharmaceutical formulations are lyophilized powders, which can be reconstituted for administration as solutions, emulsions and other mixtures. They can also be reconstituted and formulated as solids or gels.

The lyophilized powder is prepared by dissolving a antibody provided herein, in a suitable solvent. In some embodiments, the lyophilized powder is sterile. The solvent can contain an excipient which improves the stability or other pharmacological component of the powder or reconstituted solution, prepared from the powder. Excipients that can be used include, but are not limited to, dextrose, sorbital, fructose, corn syrup, xylitol, glycerin, glucose, sucrose or other suitable agent. The solvent can also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

Topical mixtures are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The antibodies provided herein can be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflations, alone or in combination with an inert carrier such as lactose. In such a case, the particles of the formulation will, in one embodiment, have diameters of less than 50 microns, in one embodiment less than 10 microns.

Antibodies provided herein can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, can be formulated as 0.01%-10% isotonic solutions, pH about 5-7, with appropriate salts.

Other routes of administration, such as transdermal patches, including iontophoretic and electrophoretic devices, and rectal administration, are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases can be used. Agents to raise the melting point of suppositories include spermaceti and wax. Rectal suppositories can be prepared either by the compressed method or by molding. The weight of a rectal suppository, in one embodiment, is about 2 to 3 gm. Tablets and capsules for rectal administration can be manufactured using the same pharmaceutically acceptable substance and by the same methods as for formulations for oral administration.

The antibodies and other compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874. In some embodiments, the anti-KIT antibodies described herein are targeted (or otherwise administered) to the bone marrow, such as in a patient having or at risk of having leukemia. In some embodiments, anti-KIT antibodies described herein are targeted (or otherwise administered) to the bone marrow, such as in a patient having or at risk of having leukemia. In some embodiments, anti-KIT antibodies described herein are targeted (or otherwise administered) to the gastrointestinal tract, such as in a patient having or at risk of having gastrointestinal stromal tumors. In some embodiments, anti-KIT antibodies described herein are targeted (or otherwise administered) to the lungs, such as in a patient having or at risk of lung cancer (e.g., small cell lung cancer). In some embodiments, anti-KIT antibodies described herein are targeted (or otherwise administered) to the brain, such as in a patient having or at risk of having neuroblastoma. In specific embodiments, an anti-KIT antibody described herein is capable of crossing the blood-brain barrier.

The antibodies provided herein can also, for example, be formulated in liposomes. Liposomes containing the molecule of interest are prepared by methods known in the art, such as described in Epstein et al. (1985) Proc. Natl. Acad. Sci. USA 82:3688; Hwang et al. (1980) Proc. Natl. Acad. Sci. USA 77:4030; and U.S. Pat. Nos. 4,485,045 and 4,544,545. Liposomes with enhanced circulation time are disclosed in U.S. Pat. No. 5,013,556.

Particularly useful immunoliposomes can be generated by the reverse phase evaporation method with a lipid composition containing phosphatidylcholine, cholesterol and PEG-derivatized phosphatidylethanolamine (PEG-PE). Liposomes are extruded through filters of defined pore size to yield liposomes with the desired diameter. Fab′ fragments of an antibody provided herein can be conjugated to the liposomes as described in Martin et al. (1982) J. Biol. Chem. 257:286-288 via a disulfide interchange reaction. A chemotherapeutic agent (such as Doxorubicin) is optionally contained within the liposome; See Gabizon et al., (1989) J. National Cancer Inst. 81(19):1484.

In one embodiment, liposomal suspensions, including tissue-targeted liposomes, such as tumor-targeted liposomes, can also be suitable as pharmaceutically acceptable carriers. These can be prepared according to methods known to those skilled in the art. For example, liposome formulations can be prepared as described in U.S. Pat. No. 4,522,811. Briefly, liposomes such as multilamellar vesicles (MLV's) can be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

An antibody described herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in, e.g., Remington's Pharmaceutical Sciences (1990) Mack Publishing Co., Easton, Pa.; Remington: The Science and Practice of Pharmacy, 21st ed. (2006) Lippincott Williams & Wilkins, Baltimore, Md.

Sustained-release preparations can also be prepared. Suitable examples of sustained-release preparations include semipermeable matrices of solid hydrophobic polymers containing an antibody described herein, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they can denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

Oral pharmaceutical dosage forms are either solid, gel or liquid. The solid dosage forms are tablets, capsules, granules, and bulk powders. Types of oral tablets include compressed, chewable lozenges and tablets which can be enteric-coated, sugar-coated or film-coated. Capsules can be hard or soft gelatin capsules, while granules and powders can be provided in non-effervescent or effervescent form with the combination of other ingredients known to those skilled in the art.

In certain embodiments, the formulations are solid dosage forms. In certain embodiments, the formulations are capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The antibody can also be mixed with other active materials which do not impair the desired action, or with materials that supplement the desired action, such as antacids, H2 blockers, and diuretics. Higher concentrations, up to about 98% by weight of the active ingredient can be included.

Provided herein is a pharmaceutical pack or kit comprising one or more containers filled with one or more of the ingredients of the pharmaceutical compositions described herein, such as one or more antibodies provided herein. Optionally associated with such container(s) can be a notice in the form prescribed by a governmental agency regulating the manufacture, use or sale of pharmaceuticals or biological products, which notice reflects approval by the agency of manufacture, use or sale for human administration.

Also provided herein are kits that can be used in the above methods. In one embodiment, a kit comprises an antibody described herein, preferably a purified antibody, in one or more containers. In a specific embodiment, the kits described herein contain a substantially isolated KIT antigen as a control. In another specific embodiment, the kits described herein further comprise a control antibody which does not react with the KIT antigen. In another specific embodiment, the kits described herein contain one or more elements for detecting the binding of a modified antibody to a KIT antigen (e.g., the antibody can be conjugated to a detectable substrate such as a fluorescent compound, an enzymatic substrate, a radioactive compound or a luminescent compound, or a second antibody which recognizes the first antibody can be conjugated to a detectable substrate). In specific embodiments, the kit can include a recombinantly produced or chemically synthesized KIT antigen. The KIT antigen provided in the kit can also be attached to a solid support. In a more specific embodiment the detecting means of the above described kit includes a solid support to which KIT antigen is attached. Such a kit can also include a non-attached reporter-labeled anti-human antibody. In this embodiment, binding of the antibody to the KIT antigen can be detected by binding of the said reporter-labeled antibody.

### 5.6 Therapeutic Methods

Provided herein are methods for impeding, preventing, treating or managing a KIT-mediated disorder or disease (e.g., cancer). Such methods comprise administering to a subject in need thereof a therapeutically effective amount of an anti-KIT antibody described herein (e.g., antibodies Ab1-Ab21). In certain aspects, also provided herein are methods for preventing, treating or managing one or more symptoms of a KIT-mediated disorder or disease.

In specific embodiments, methods described herein for treating a KIT-mediated disorder or disease provide for the reduction or amelioration of the progression, severity, and/or duration of a KIT-mediated disorder or disease (e.g., cancer, inflammatory condition, or fibrosis) resulting from the administration of one or more therapies (including, but not limited to, the administration of one or more prophylactic or therapeutic agents, such as an anti-KIT antibody described herein). In further specific embodiments, methods described herein for treating a KIT-mediated disorder or disease relate to reducing one or more symptoms of a KIT-mediated disorder or disease. In specific embodiments, an antibody described herein, such as any one of antibodies Ab1-Ab21 is for use in treating or managing a KIT-mediated disorder (e.g., cancer). In a particular embodiment, provided herein is an an antibody for use in treating or managing a KIT-mediated disorder (e.g., cancer), wherein the antibody comprises (i) a VL chain region having the amino acid sequence of SEQ ID NO: 176, SEQ ID NO: 176 starting at the second amino acid residue, SEQ ID NO: 350, SEQ ID NO: 963, SEQ ID NO: 964, or SEQ ID NO: 328, and (ii) a VH chain region having the amino acid sequence of SEQ ID NO: 188 or 329. In another particular embodiment, provided herein is an antibody for use in treating or managing a KIT-mediated disorder (e.g., cancer), wherein the antibody comprises (i) a VL chain region comprising a VL CDR1, VL CDR2, and VL CDR3, wherein the VL CDR1 has the amino acid sequence of SEQ ID NO: 2 (VL CDR1 of Ab1), the VL CDR2 has the amino acid sequence of SEQ ID NO: 14 (VL CDR2 of Ab1), and the VL CDR3 has the amino acid sequence of SEQ ID NO: 26 (VL CDR3 of Ab1) or SEQ ID NO: 332 (VL CDR3 of Ab21), respectively; and (ii) a VH chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively: In a specific embodiment, the antibody used in the methods described herein is internalized by the cell to which it binds. In a particular embodiment, a conjugate is used in the methods described herein, wherein the conjugate comprises an antibody described herein (e.g., Ab1, Ab21, or an antibody comprising the VL CDRs and VH CDRs of Ab1 or Ab21), or a KIT-binding fragment thereof. In a specific embodiment, the conjugate comprises an antibody described herein (e.g., Ab1, Ab21, or an antibody comprising the VL CDRs and VH CDRs of Ab1 or Ab21), or a KIT-binding fragment thereof, linked, covalently or non-covalently, to a therapeutic agent, such as a toxin. In a certain embodiment, the conjugate used in the methods described herein is internalized into a cell to which it binds.

A KIT-mediated disorder or disease is one which is completely or partially caused by or is the result of KIT. In one aspect, a KIT-mediated disorder or disease can be known to one of skill in the art or can be ascertained by one of skill in the art. In certain aspects, a KIT-mediated disorder or disease can be characterized by gain-of-function KIT activity or overexpression of KIT. In one embodiment, a KIT-mediated disorder or disease is completely or partially caused by or is the result of gain-of-function KIT activity or overexpression of KIT. In certain embodiments, the gain-of-function KIT activity can occur independent of KIT ligand (e.g., SCF) binding KIT receptor. In particular aspects, high or overexpression of KIT in a cell refers to an expression level which is at least about 10%, 20%, 30%, 40%, 50%, 60%, 70%, 80%, 90%, or 100% more than the expression level of a reference cell known to have normal KIT expression or KIT activity or more than the average expression level of KIT in a population of cells or samples known to have normal KIT expression or KIT activity. Expression levels of KIT can be assessed by methods described herein or known to one of skill in the art (e.g., Western blotting or immunohistorychemistry). In particular embodiments, a KIT-mediated disorder or disease is characterized by KIT activity which is higher than normal KIT activity and leads to cellular transformation, neoplasia, and tumorogenesis.

Non-limiting examples of disorders or KIT-mediated disorders or diseases include cancers such as breast cancer, leukemia (e.g., chronic myelogenous leukemia, acute myeloid leukemia, mast cell leukemia), lung cancer (e.g., small cell lung cancer), neuroblastoma, gastrointestinal stromal tumors (GIST), melanoma, colorectal cancer, and germ cell tumors (e.g., seminoma). In a particular embodiment, a cancer which is treated or managed by the methods provided herein is characterized by a gain-of-function KIT mutation or overexpression of KIT.

In a specific embodiment, a method described herein is for treating cancer, wherein said method comprises administering to a subject in need thereof a therapeutically effective amount of antibody Ab1, or an antigen binding fragment thereof (e.g., KIT-binding fragment thereof), or a conjugate comprising Ab1 or a KIT-binding fragment thereof linked, covalently or non-covalently, to a therapeutic agent. In a specific embodiment, a method described herein is for treating cancer, wherein said method comprises administering to a subject in need thereof a therapeutically effective amount of an antibody comprising the VL CDRs and VH CDRs of Ab1, or a conjugate comprising an antibody comprising the VL CDRs and VH CDRs of Ab1, linked, covalently or non-covalently, to a therapeutic agent. In certain aspects, also provided herein are methods for preventing, treating or managing one or more symptoms of cancer, wherein said methods comprise administering to a subject in need thereof a therapeutically effective amount of antibody Ab1, or an antigen binding fragment thereof (e.g., KIT-binding fragment thereof), or a conjugate comprising Ab1 or a KIT-binding fragment thereof linked, covalently or non-covalently, to a therapeutic agent. In certain aspects, also provided herein are methods for preventing, treating or managing one or more symptoms of cancer, wherein said methods comprise administering to a subject in need thereof a therapeutically effective amount of an antibody comprising the VL CDRs and VH CDRs of Ab1, or a conjugate comprising such antibody comprising the VL CDRs and VH CDRs of Ab1, linked, covalently or non-covalently, to a therapeutic agent. In a specific embodiment, Ab1 comprises a VL domain comprising the amino acid sequence of SEQ ID NO: 176 or SEQ ID NO: 176 starting with the second amino acid residue of SEQ ID NO: 176. In a specific embodiment, Ab1 comprises a VL domain comprising the amino acid sequence of SEQ ID NO: 350.

In a specific embodiment, a method described herein is for treating cancer, wherein said method comprises administering to a subject in need thereof a therapeutically effective amount of antibody Ab21, or an antigen binding fragment thereof (e.g., KIT-binding fragment thereof), or a conjugate comprising Ab21 or a KIT-binding fragment thereof linked, covalently or non-covalently, to a therapeutic agent. In certain aspects, also provided herein are methods for preventing, treating or managing one or more symptoms of cancer, wherein said methods comprise administering to a subject in need thereof a therapeutically effective amount of antibody Ab21, or an antigen binding fragment thereof (e.g., KIT-binding fragment thereof), or a conjugate comprising Ab21 or a KIT-binding fragment thereof linked, covalently or non-covalently, to a therapeutic agent. In a specific embodiment, a method described herein is for treating cancer, wherein said method comprises administering to a subject in need thereof a therapeutically effective amount of an antibody comprising the VL CDRs and VH CDRs of Ab21, or a conjugate comprising such antibody comprising the VL CDRs and VH CDRs of Ab21, linked, covalently or non-covalently, to a therapeutic agent. In certain aspects, also provided herein are methods for preventing, treating or managing one or more symptoms of cancer, wherein said methods comprise administering to a subject in need thereof a therapeutically effective amount of an antibody comprising the VL CDRs and VH CDRs of Ab21, or a conjugate comprising such antibody comprising the VL CDRs and VH CDRs of Ab21, linked, covalently or non-covalently, to a therapeutic agent.

In a specific embodiment, a method described herein is for treating GIST, wherein said method comprises administering to a subject in need thereof a therapeutically effective amount of antibody Ab1, or an antigen binding fragment thereof, or a conjugate comprising Ab1 or a KIT-binding fragment thereof linked, covalently or non-covalently, to a therapeutic agent. In certain aspects, also provided herein are methods for preventing, treating or managing one or more symptoms of GIST, wherein said methods comprise administering to a subject in need thereof a therapeutically effective amount of antibody Ab1, or an antigen binding fragment thereof, or a conjugate comprising Ab1 or a KIT-binding fragment thereof linked, covalently or non-covalently, to a therapeutic agent. In a specific embodiment, Ab1 comprises a VL domain comprising the amino acid sequence of SEQ ID NO: 350, or SEQ ID NO: 176 or SEQ ID NO: 176 starting with the second amino acid residue of SEQ ID NO: 176. In a specific embodiment, a method described herein is for treating GIST, wherein said method comprises administering to a subject in need thereof a therapeutically effective amount of an antibody comprising the VL CDRs and VH CDRs of Ab1, or a conjugate comprising such antibody comprising the VL CDRs and VH CDRs of Ab1, linked, covalently or non-covalently, to a therapeutic agent. In certain aspects, also provided herein are methods for preventing, treating or managing one or more symptoms of GIST, wherein said methods comprise administering to a subject in need thereof a therapeutically effective amount of an antibody comprising the VL CDRs and VH CDRs of Ab1, or a conjugate comprising such antibody comprising the VL CDRs and VH CDRs of Ab1, linked, covalently or non-covalently, to a therapeutic agent.

In a specific embodiment, a method described herein is for treating GIST, wherein said method comprises administering to a subject in need thereof a therapeutically effective amount of antibody Ab21, or an antigen binding fragment thereof, or a conjugate comprising Ab21 or a KIT-binding fragment thereof linked, covalently or non-covalently, to a therapeutic agent. In a specific embodiment, a method described herein is for treating GIST, wherein said method comprises administering to a subject in need thereof a therapeutically effective amount of an antibody comprising the VL CDRs and VH CDRs of Ab21, or a conjugate comprising such antibody or a KIT-binding fragment thereof, linked, covalently or non-covalently, to a therapeutic agent. In certain aspects, also provided herein are methods for preventing, treating or managing one or more symptoms of GIST, wherein said methods comprise administering to a subject in need thereof a therapeutically effective amount of antibody Ab21 or an antigen binding fragment thereof, or a conjugate comprising Ab21 or a KIT-binding fragment thereof linked, covalently or non-covalently, to a therapeutic agent. In certain aspects, also provided herein are methods for preventing, treating or managing one or more symptoms of GIST, wherein said methods comprise administering to a subject in need thereof a therapeutically effective amount of an antibody comprising the VL CDRs and VH CDRS of Ab21, or a conjugate comprising the VL CDRs and VH CDRS of Ab21 linked, covalently or non-covalently, to a therapeutic agent.

In a specific embodiment, a method described herein is for treating lung cancer (e.g., small cell lung carcinoma), wherein said method comprises administering to a subject in need thereof a therapeutically effective amount of antibody Ab1, or an antigen binding fragment thereof, or a conjugate comprising Ab1 or a KIT-binding fragment thereof linked, covalently or non-covalently, to a therapeutic agent. In certain aspects, also provided herein are methods for preventing, treating or managing one or more symptoms of lung cancer (e.g., small cell lung carcinoma), wherein said methods comprise administering to a subject in need thereof a therapeutically effective amount of antibody Ab1, or an antigen binding fragment thereof, or a conjugate comprising Ab1 or a KIT-binding fragment thereof linked, covalently or non-covalently, to a therapeutic agent. In a specific embodiment, Ab1 comprises a VL domain comprising the amino acid sequence of SEQ ID NO: 350, SEQ ID NO: 963, SEQ ID NO: 176 or SEQ ID NO: 176 starting with the second amino acid residue of SEQ ID NO: 176. In a specific embodiment, a method described herein is for treating lung cancer (e.g., small cell lung carcinoma), wherein said method comprises administering to a subject in need thereof a therapeutically effective amount of an antibody comprising the VL CDRs and VH CDRs of Ab1, or a conjugate comprising such antibody comprising the VL CDRs and VH CDRs of Ab1, linked, covalently or non-covalently, to a therapeutic agent. In certain aspects, also provided herein are methods for preventing, treating or managing one or more symptoms of lung cancer (e.g., small cell lung carcinoma), wherein said methods comprise administering to a subject in need thereof a therapeutically effective amount of an antibody comprising the VL CDRs and VH CDRs of Ab1, or a conjugate comprising such antibody comprising the VL CDRs and VH CDRs of Ab1, linked, covalently or non-covalently, to a therapeutic agent.

In a specific embodiment, a method described herein is for treating lung cancer (e.g., small cell lung carcinoma), wherein said method comprises administering to a subject in need thereof a therapeutically effective amount of antibody Ab21, or an antigen binding fragment thereof, or a conjugate comprising Ab21 or a KIT-binding fragment thereof linked, covalently or non-covalently, to a therapeutic agent. In certain aspects, also provided herein are methods for preventing, treating or managing one or more symptoms of lung cancer (e.g., small cell lung carcinoma), wherein said methods comprise administering to a subject in need thereof a therapeutically effective amount of antibody Ab21, or an antigen binding fragment thereof, or a conjugate comprising Ab21 or a KIT-binding fragment thereof linked, covalently or non-covalently, to a therapeutic agent. In a specific embodiment, a method described herein is for treating lung cancer (e.g., small cell lung carcinoma), wherein said method comprises administering to a subject in need thereof a therapeutically effective amount of an antibody comprising the VL CDRs and VH CDRs of Ab21, or a conjugate comprising such antibody comprising the VL CDRs and VH CDRs of Ab21, linked, covalently or non-covalently, to a therapeutic agent. In certain aspects, also provided herein are methods for preventing, treating or managing one or more symptoms of lung cancer (e.g., small cell lung carcinoma), wherein said methods comprise administering to a subject in need thereof a therapeutically effective amount of an antibody comprising the VL CDRs and VH CDRs of Ab21, or a conjugate comprising such antibody comprising the VL CDRs and VH CDRs of Ab21, linked, covalently or non-covalently, to a therapeutic agent.

Cancer relates to the physiological condition in mammals that is typically characterized by unregulated cell growth. The cancer treated in accordance with the methods described herein is any type of cancer which comprises cancer or tumor cells expressing KIT or a mutated form thereof, which can be confirmed by any histologically or cytologically method known to one of skill in the art.

In certain embodiments, a cancer is metastatic. In certain embodiments, a cancer is an advanced cancer which has spread outside the site or organ of origin, either by local invasion or metastasis.

In particular embodiments, a cancer is a recurrent cancer which has regrown, either at the initial site or at a distant site, after a response to initial therapy (e.g., after surgery to remove the tumor and adjuvant therapy following surgery). In some embodiments, a cancer is a refractory cancer which progresses even though an anti-tumor agent, such as a chemotherapeutic agent, is being administered, or has been administered, to the cancer patient. A non-limiting example of a refractory cancer is one which is refractory to a tyrosine kinase inhibitor, such as GLEEVEC® (imatinib mesylate), SUTENT® (SU11248 or sunitinib), IRESSA™ (gefitinib), TARCEVA® (erlotinib), NEXAVAR® (sorafenib), or VOTRIENT™ (pazopanib). In some embodiments, a cancer is a refractory cancer which progresses even though radiation or chemotherapy is being administered, or has been administered, to the cancer patient.

In specific embodiments, provided herein are methods for treating a refractory cancer in a patient in need thereof comprising administering to the patient a therapeutically effective amount of an antibody described herein (e.g., antibody Ab1, antibody Ab21, an antibody comprising the VL CDRs and/or VH CDRs of antibody Ab1, an antibody comprising the VL CDRs and/or VH CDRs of antibody Ab21, or an antigen-binding fragment of any such antibody), wherein the refractory cancer is refractory or resistant to an anti-cancer agent such as a tyrosine kinase inhibitor (e.g., GLEEVEC® (imatinib mesylate) or SUTENT® (SU11248 or Sunitinib)). Other non-limiting examples of tyrosine kinse inhibitors include 706 and AMNI07 (nilotinib). RAD00I, PKC412, gefitinib (IRESSAT™), erlotinib (TARCEVA®), sorafenib (NEXAVAR®), pazopanib (VOTRIENT™), axitinib, bosutinib, cediranib (RECENTIN®), SPRYCEL® (dasatinib), lapatinib (TYKERB®), lestaurtinib, neratinib, nilotinib (TASIGNA®), semaxanib, toceranib (PALLADIA™), vandetanib (ZACTIMA™), and vatalanib. In certain embodiments, the refractory cancer was initially responsive to an anti-cancer agent, such as a tyrosine kinase inhibitor (e.g., GLEEVEC® or SU11248 (Sunitinib)), but has developed resistance the anti-cancer agent. In certain embodiments, a subject has one or more mutations in KIT that confers resistance to an anti-cancer agent such as a tyrosine kinase inhibitor. In a specific embodiment, provided herein is a method for treating a refractory cancer in a patient in need thereof comprising administering to the patient a therapeutically effective amount of an antibody comprising the VL CDRs and/or VH CDRs of antibody Ab1 or Ab21, an antigen-binding fragment of any such antibody, or a conjugate comprising such antibody or antigen-binding fragment, wherein the refractory cancer is refractory or resistant to an anti-cancer agent such as a tyrosine kinase inhibitor (e.g., GLEEVEC® (imatinib mesylate) or SUTENT® (SU11248 or Sunitinib)).

In particular embodiments, an antibody described herein (e.g., Ab1, Ab21, an antibody comprising the VL CDRs and/or VH CDRs of Ab1, an antibody comprising the VL CDRs and/or VH CDRs of Ab21, or an antigen-binding fragment of any such antibody) is administered to a patient who has previously received, or is currently receiving, one or more anti-cancer therapies, for example, a tyrosine kinase inhibitor, e.g., GLEEVEC® (imatinib mesylate), SUTENT® (SU11248 or sunitinib), IRESSA™ (gefitinib), TARCEVA® (erlotinib), NEXAVAR® (sorafenib), or VOTRIENT™ (pazopanib). In other particular embodiments, an antibody described herein is administered to a patient who is, or is suspected of being, resistant or refractory to an anti-cancer therapy, for example, a tyrosine kinase inhibitor, e.g., GLEEVEC® (imatinib mesylate), SUTENT® (SU11248 or sunitinib), IRESSA™ (gefitinib), TARCEVA® (erlotinib), NEXAVAR® (sorafenib), or VOTRIENT™ (pazopanib). In particular embodiments, an antibody comprising the VL CDRs and VH CDRs of antibody Ab1 or Ab21, or a conjugate comprising such antibody is administered to a patient who has previously received, or is currently receiving, one or more anti-cancer therapies, for example, a tyrosine kinase inhibitor, e.g., GLEEVEC® (imatinib mesylate), SUTENT® (SU11248 or sunitinib).

In particular embodiments, an antibody described herein (e.g., Ab1, Ab21, an antibody comprising the VL CDRs and VH CDRs of Ab1, or an antibody comprising the VL CDRs and VH CDRs of Ab21) is administered to a patient who has previously received, or is currently receiving, one or more anti-cancer therapies, for example, an anti-growth factor receptor antibody (e.g., anti-HER2 antibody, anti-EGFR antibody, anti-VEGFR antibody, or anti-KIT antibody) or anti-growth factor antibody (e.g., anti-EGF antibody, anti-VEGF antibody). In other particular embodiments, an antibody described herein is administered to a patient who is, or is suspected of being, resistant or refractory to an anti-cancer therapy, for example, an anti-growth factor receptor antibody (e.g., anti-HER2 antibody, anti-EGFR antibody, anti-VEGFR antibody, or anti-KIT antibody) or anti-growth factor antibody (e.g., anti-EGF antibody, anti-VEGF antibody).

In a particular embodiment, a method described herein for treating or managing cancer in a subject in need thereof, can achieve at least one, two, three, four or more of the following effects due to administration of a therapeutically effective amount of an anti-KIT antibody described herein: (i) the reduction or amelioration of the severity of cancer (e.g., leukemia, lung cancer, or gastrointestinal stromal cancer) and/or one or more symptoms associated therewith; (ii) the reduction in the duration of one or more symptoms associated with a cancer (e.g., leukemia, lung cancer, or gastrointestinal stromal cancer); (iii) the prevention in the recurrence of a tumor (e.g., lung tumor or gastrointestinal stromal tumor); (iv) the regression of a cancer (e.g., leukemia, lung cancer, or gastrointestinal stromal tumor) and/or one or more symptoms associated therewith; (v) the reduction in hospitalization of a subject; (vi) the reduction in hospitalization length; (vii) the increase in the survival of a subject; (viii) the inhibition of the progression of a cancer (e.g., leukemia, lung cancer, or gastrointestinal stromal tumor) and/or one or more symptoms associated therewith; (ix) the enhancement or improvement of the therapeutic effect of another therapy (e.g., surgery, radiation, chemotherapy, or another tyrosine kinase inhibitor); (x) a reduction or elimination in the cancer cell population (e.g., leukemia cell population, lung cancer cell population, gastrointestinal stromal tumor cell population); (xi) a reduction in the growth of a tumor or neoplasm; (xii) a decrease in tumor size (e.g., volume or diameter); (xiii) a reduction in the formation of a newly formed tumors; (xiv) eradication, removal, or control of primary, regional and/or metastatic cancer; (xv) ease in removal of a tumor by reducing tumor and/or edema-related vascularization prior to surgery; (xvi) a decrease in the number or size of metastases; (xvii) a reduction in mortality; (xviii) an increase in tumor-free survival rate of patients; (xvix) an increase in relapse-free survival; (xx) an increase in the number of patients in remission; (xxi) a decrease in hospitalization rate; (xxii) the size of the tumor is maintained and does not increase or increases by less than the increase of a tumor after administration of a standard therapy as measured by conventional methods available to one of skill in the art, such as computed tomography (CT) scan, magnetic resonance imaging (MRI), dynamic contrast-enhanced MRI (DCE-MRI), or a positron emission tomography (PET) scan; (xxiii) the prevention of the development or onset of one or more symptoms associated cancer; (xxiv) an increase in the length of remission in patients; (xxv) the reduction in the number of symptoms associated with cancer; (xxvi) an increase in symptom-free survival of cancer patients; (xxvii) a decrease in the concentration of one or more inflammatory mediators (e.g., cytokines or interleukins) in biological specimens (e.g., plasma, serum, cerebral spinal fluid, urine, or any other biofluids) of a subject with a cancer (e.g., leukemia, lung cancer, or gastrointestinal stromal cancer); (xxviii) a decrease in circulating tumor cells (CTCs) in the blood of a subject with cancer (e.g., leukemia, lung cancer, or gastrointestinal stromal cancer); (xxix) inhibition or decrease in tumor metabolism or perfusion; and (xxx) improvement in the quality of life as assessed by methods well known in the art, e.g., questionnaires.

Other non-limiting examples of KIT-mediated disorders or diseases include systemic mast cell disorders (e.g., mastocytosis), hematologic disorders, fibrosis (e.g., idiopathic pulmonary fibrosis (TPF), scleroderma, myelofibrosis) and inflammatory conditions such as asthma, rheumatoid arthritis, inflammatory bowel disease, and allergic inflammation.

In a particular embodiment, a method described herein for treating or managing a KIT-mediated disorder, e.g., fibrosis or an inflammatory condition (e.g., asthma, rheumatoid arthritis, inflammatory bowel disease, and allergic inflammation), in a subject in need thereof, can achieve at least one, two, three, four or more of the following effects due to administration of a therapeutically effective amount of an anti-KIT antibody described herein: (i) the reduction or amelioration of the severity of fibrosis or an inflammatory condition (e.g., asthma, rheumatoid arthritis, inflammatory bowel disease, and allergic inflammation) and/or one or more symptoms associated therewith; (ii) the reduction in the duration of one or more symptoms associated with fibrosis or an inflammatory condition (e.g., asthma, rheumatoid arthritis, inflammatory bowel disease, and allergic inflammation); (iii) the prevention in the recurrence of fibrosis or an inflammatory condition (e.g., asthma, rheumatoid arthritis, inflammatory bowel disease, and allergic inflammation); (iv) the reduction in hospitalization of a subject; (v) the reduction in hospitalization length; (vi) the inhibition of the progression of fibrosis or an inflammatory condition (e.g., asthma, rheumatoid arthritis, inflammatory bowel disease, and allergic inflammation) and/or one or more symptoms associated therewith; (vii) the enhancement or improvement of the therapeutic effect of another therapy (e.g., anti-inflammatory therapy such as steriods); (viii) an increase in the number of patients in remission (i.e., a time period characterized by no or minimal symptoms associated with the inflammatory condition); (ix) an increase in the length of remission in patients; (x) a decrease in hospitalization rate; (xi) the reduction in the number of symptoms associated with fibrosis or an inflammatory condition (e.g., asthma, rheumatoid arthritis, inflammatory bowel disease, and allergic inflammation); (xii) a decrease in the concentration of one or more inflammatory mediators (e.g., cytokines or interleukins) in biological specimens (e.g., plasma, serum, cerebral spinal fluid, urine, or any other biofluids) of a subject with fibrosis or an inflammatory condition (e.g., asthma, rheumatoid arthritis, inflammatory bowel disease, and allergic inflammation); and (xiii) improvement in the quality of life as assessed by methods well known in the art, e.g., questionnaires.

In a particular embodiment, provided herein is a method for treating or managing a KIT-mediated disorder, e.g., fibrosis or an inflammatory condition (e.g., asthma, rheumatoid arthritis, inflammatory bowel disease, and allergic inflammation), in a subject in need thereof, wherein said method comprises administering to said subject a therapeutically effective amount of an antibody described herein, such as Ab1, Ab21, an antibody comprising the VL CDRs and VH CDRs of Ab1, or an antibody comprising the VL CDRs and VH CDRs of Ab21.

In certain embodiments, an anti-KIT antibody described herein my be administered by any suitable method to a subject in need thereof. Non-limiting examples of administration methods include mucosal, intradermal, intravenous, intratumoral, subcutaneous, intramuscular delivery and/or any other method of physical delivery described herein or known in the art. In one embodiment, an anti-KIT antibody or a pharmaceutical composition thereof is administered systemically (e.g., parenterally) to a subject in need thereof. In another embodiment, an anti-KIT antibody or a pharmaceutical composition thereof is administered locally (e.g., intratumorally) to a subject in need thereof. Each dose may or may not be administered by an identical route of administration. In some embodiments, an anti-KIT antibody described herein can be administered via multiple routes of administration simultaneously or subsequently to other doses of the same or a different an anti-KIT antibody described herein.

When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof. In certain embodiments, an anti-KIT antibody described herein is administered prophylactically or therapeutically to a subject. An anti-KIT antibody described herein can be prophylactically or therapeutically administered to a subject so as to prevent, lessen or ameliorate a KIT-mediated disorder or disease (e.g., cancer, inflammatory condition, fibrosis) or symptom thereof.

The dosage and frequency of administration of an anti-KIT antibody described herein or a pharmaceutical composition thereof is administered to a subject in need thereof in accordance with the methods for treating a KIT-mediated disorder or disease provided herein will be efficacious while minimizing side effects. The exact dosage of an anti-KIT antibody described herein to be administered to a particular subject or a pharmaceutical composition thereof can be determined by a practitioner, in light of factors related to the subject that requires treatment. Factors which can be taken into account include the severity of the disease state, general health of the subject, age, and weight of the subject, diet, time and frequency of administration, combination(s) with other therapeutic agents or drugs, reaction sensitivities, and tolerance/response to therapy. The dosage and frequency of administration of an anti-KIT antibody described herein or a pharmaceutical composition thereof can be adjusted over time to provide sufficient levels of the anti-KIT antibody or to maintain the desired effect.

The precise dose to be employed in the formulation will also depend on the route of administration, and the seriousness of a KIT-mediated disorder or disease (e.g., cancer, inflammatory condition, fibrosis), and should be decided according to the judgment of the practitioner and each patient's circumstances.

Effective doses can be extrapolated from dose-response curves derived from in vitro or animal model test systems.

In one embodiment, for the anti-KIT antibodies described herein, the dosage administered to a patient, to manage a KIT-mediated disorder or disease (e.g., cancer, inflammatory condition, fibrosis) is typically 0.1 mg/kg to 100 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of the antibodies described herein can be reduced by enhancing uptake and tissue penetration of the antibodies by modifications such as, for example, lipidation.

In one embodiment, approximately 100 mg/kg or less, approximately 75 mg/kg or less, approximately 50 mg/kg or less, approximately 25 mg/kg or less, approximately 10 mg/kg or less, approximately 5 mg/kg or less, approximately 1 mg/kg or less, approximately 0.5 mg/kg or less, or approximately 0.1 mg/kg or less of an anti-KIT antibody described herein is administered to manage a KIT-mediated disorder or disease (e.g., cancer, inflammatory condition, fibrosis).

In some embodiments, an effective amount of an antibody provided herein is from about 0.1 mg/kg (mg of antibody per kg weight of a subject) to about 100 mg/kg. In certain embodiments, an effective amount of an antibody provided therein is about 0.1 mg/kg, about 0.5 mg/kg, about 1 mg/kg, 3 mg/kg, 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg about 90 mg/kg or about 100 mg/kg (or a range therein). In specific embodiments, an “effective amount” of an anti-KIT antibody described herein refers to an amount of an anti-KIT antibody described herein which is sufficient to achieve at least one, two, three, four or more of the following effects: (i) the reduction or amelioration of the severity of a KIT-mediated disorder or disease (e.g., cancer, inflammatory condition, fibrosis) and/or one or more symptoms associated therewith; (ii) the reduction in the duration of one or more symptoms associated with a KIT-mediated disorder or disease (e.g., cancer, inflammatory condition, fibrosis); (iii) the prevention in the recurrence of a tumor (e.g., gastrointestinal stromal tumor); (iv) the regression of a KIT-mediated disorder or disease (e.g., cancer, inflammatory condition, fibrosis) and/or one or more symptoms associated therewith; (v) the reduction in hospitalization of a subject; (vi) the reduction in hospitalization length; (vii) the increase in the survival of a subject; (viii) the inhibition of the progression of a KIT-mediated disorder or disease (e.g., cancer, inflammatory condition, fibrosis) and/or one or more symptoms associated therewith; (ix) the enhancement or improvement of the therapeutic effect of another therapy; (x) a reduction or elimination in the cancer cell population (e.g., leukemia cell population, lung cancer cell population, gastrointestinal stromal cancer cell population); (xi) a reduction in the growth of a tumor or neoplasm; (xii) a decrease in tumor size (e.g., volume or diameter); (xiii) a reduction in the formation of a newly formed tumors; (xiv) eradication, removal, or control of primary, regional and/or metastatic cancer; (xv) ease in removal of a tumor by reducing tumor and/or edema-related vascularization prior to surgery; (xvi) a decrease in the number or size of metastases; (xvii) a reduction in mortality; (xviii) an increase in tumor-free survival rate of patients; (xvix) an increase in relapse-free survival; (xx) an increase in the number of patients in remission; (xxi) a decrease in hospitalization rate; (xxii) the size of the tumor is maintained and does not increase or increases by less than the increase of a tumor after administration of a standard therapy as measured by conventional methods available to one of skill in the art, such as computed tomography (CT) scan, magnetic resonance imaging (MRI), dynamic contrast-enhanced MRI (DCE-MRI), or a positron emission tomography (PET) scan; (xxiii) the prevention of the development or onset of one or more symptoms associated cancer; (xxiv) an increase in the length of remission in patients; (xxv) the reduction in the number of symptoms associated with cancer; (xxvi) an increase in symptom-free survival of cancer patients; (xxvii) a decrease in the concentration of one or more inflammatory mediators (e.g., cytokines or interleukins) in biological specimens (e.g., plasma, serum, cerebral spinal fluid, urine, or any other biofluids) of a subject with a KIT-mediated disorder or disease (e.g., cancer, inflammatory condition, fibrosis); (xxviii) a decrease in circulating tumor cells (CTCs) in the blood of a subject with cancer; (xxix) inhibition or decrease in tumor metabolism or perfusion; and (xxx) improvement in the quality of life as assessed by methods well known in the art, e.g., questionnaires. In some embodiments, “effective amount” as used herein also refers to the amount of an antibody described herein to achieve a specified result (e.g., inhibition of one or more KIT biological activities of a cell, such as inhibition of cell proliferation).

In some embodiments, an anti-KIT antibody described herein is administered as necessary, e.g., weekly, biweekly (i.e., once every two weeks), monthly, bimonthly, trimonthly, etc., as determined by a physician.

In some embodiments, a single dose of an anti-KIT antibody described herein is administered one or more times to a patient to impede, prevent, manage, treat and/or ameliorate a KIT-mediated disorder or disease (e.g., cancer, inflammatory condition, fibrosis).

In particular embodiments, an anti-KIT antibody or pharmaceutical composition thereof is administered to a subject in accordance with the methods for treating a KIT-mediated disorder or disease (e.g., cancer, inflammatory condition, fibrosis) presented herein in cycles, wherein the anti-KIT antibody or pharmaceutical composition is administered for a period of time, followed by a period of rest (i.e., the anti-KIT antibody or pharmaceutical composition is not administered for a period of time).

Also, presented herein are combination therapies for the treatment of a KIT-mediated disorder or disease (e.g., cancer, inflammatory condition, fibrosis) which involve the administration of an anti-KIT antibody described herein in combination with one or more additional therapies to a subject in need thereof. In a specific embodiment, presented herein are combination therapies for the treatment of a KIT-mediated disorder or disease (e.g., cancer, inflammatory condition, fibrosis) which involve the administration of a therapeutically effective amount of an anti-KIT antibody described herein in combination with a therapeutically effective amount of another therapy to a subject in need thereof.

In combination therapies, one or more anti-KIT antibodies provided herein can be administered prior to, concurrently with, or subsequent to the administration of one or more additional therapies (e.g., agents, surgery, or radiation) for use in treating, managing, and/or ameoliorating a KIT-mediated disorder or disease (e.g., cancer, inflammatory condition, fibrosis). The use of the term “in combination” does not restrict the order in which one or more anti-KIT antibodies and one or more additional therapies are administered to a subject.

In another specific embodiment, presented herein are combination therapies for the treatment of a KIT-mediated disorder or disease (e.g., cancer, inflammatory condition, fibrosis) which involve the administration of an amount of an anti-KIT antibody described herein in combination with an amount of another therapy to a subject in need thereof, wherein the combination therapies result in a synergistic effect. In certain embodiments, the combination therapies result in an additive effect.

In a specific embodiment, presented herein are combination therapies for the treatment of cancer which involve the administration of an amount of an anti-KIT antibody described herein in combination with an amount of another therapy (e.g., surgery, radiation, stem cell transplantation, or chemotherapy) to a subject in need thereof, wherein the combination therapies result in a synergistic effect. In another specific embodiment, the combination therapies result in an additive effect.

In a specific embodiment, presented herein are combination therapies for the treatment of an inflammatory condition which involve the administration of an amount of an anti-KIT antibody described herein in combination with an amount of another therapy (e.g., anti-inflammatory therapy, for example, steroid therapy) to a subject in need thereof, wherein the combination therapies result in a synergistic effect. In another specific embodiment, the combination therapies result in an additive effect.

Non-limiting examples of another therapy for use in combination with antibodies described herein (e.g., Ab1, Ab21, an antibody comprising the VL CDRs and VH CDRs of Ab1, or an antibody comprising the VL CDRs and VH CDRs of Ab21) include, another anti-KIT antibody that immunospecifically bind to a different epitope of KIT, one or more other antibodies (e.g., anti-HER2 antibody, anti-EGFR antibody, anti-VEGF antibody), anti-inflammatory therapy, chemotherapy (e.g., microtubule disassembly blocker, antimetabolite, topisomerase inhibitor, and DNA crosslinker or damaging agent), radiation, surgery, and tyrosine kinase inhibitors (e.g., gefitinib (IRESSA™), erlotinib (TARCEVA®), sorafenib (NEXAVAR®), pazopanib (VOTRIENTT™), axitinib, bosutinib, cediranib (RECENTIN®), SPRYCEL® (dasatinib), lapatinib (TYKERB®), lestaurtinib, neratinib, nilotinib (TASIGNA®), semaxanib, toceranib (PALLADIA™), vandetanib (ZACTIMA™), imatinib mesylate (GLEEVEC®), sunitinib (SUTENT®), and vatalanib). In a specific embodiment, provided herein is a method of treating a KIT-mediated disorder (e.g., cancer) comprising administering to a subject in need thereof an antibody comprising the VL CDRs of antibody Ab1 or a conjugate comprising such antibody, in combination with imatinib mesylate (GLEEVEC®) or sunitinib (SUTENT®). In a specific embodiment, provided herein is a method of treating a KIT-mediated disorder (e.g., cancer) comprising administering to a subject in need thereof an antibody comprising the VL CDRs of antibody Ab21 or a conjugate comprising such antibody, in combination with imatinib mesylate (GLEEVEC®) or sunitinib (SUTENT®). In a specific embodiment, provided herein is a method of treating a KIT-mediated disorder (e.g., cancer) comprising administering to a subject in need thereof antibody Ab1 or Ab21, an antigen-binding fragment thereof, or a conjugate comprising such antibody, in combination with imatinib mesylate (GLEEVEC®) or sunitinib (SUTENT®).

### 5.7 Diagnostic Methods

Labeled or otherwise detectable antibodies, which immunospecifically bind to a KIT antigen (e.g., the D4/D5 region of KIT, for example, human KIT) can be used for diagnostic purposes to detect, diagnose, or monitor a KIT-mediated disease. In certain aspects, diagnostic methods provided herein are performed, in vitro. In other certain aspects, diagnostic methods provided herein are performed, ex vivo.

Provided herein are methods for detecting KIT expression in samples obtained from patients with a KIT-mediated disorder or disease. In a particular embodiment, a method for detecting KIT expression in a sample obtained from a patient comprises contacting the sample with an anti-KIT antibody (e.g., antibody Ab1 or Ab21, an antibody comprising the VL CDRs and/or VH CDRs of antibody Ab1 or Ab21, an antigen-binding fragment of any such antibody, or a conjugate comprising any such antibody or antigen-binding fragment) described herein and detecting the expression level of KIT in the samples. Methods for detection are known to one of skill in the art.

In certain aspects, provided herein are methods for diagnosing a patient with a KIT-mediated disorder or disease. In a certain aspect, a method for diagnosing a subject with a KIT-mediated disorder or disease comprises contacting a sample obtained from the subject with an anti-KIT antibody described herein (e.g., antibody Ab1 or Ab21, an antibody comprising the VL CDRs and/or VH CDRs of antibody Ab1 or Ab21, an antigen-binding fragment of any such antibody, or a conjugate comprising any such antibody or antigen-binding fragment) and detecting the expression level of KIT in the sample.

In certain aspects, provided herein are methods for the detection of a KIT-mediated disease comprising: (a) assaying the expression of a KIT antigen in cells or a tissue sample of a subject using one or more antibodies described herein; and (b) comparing the level of the KIT antigen with a control level, e.g., levels in normal tissue samples (e.g., from a patient not having a KIT-mediated disease, or from the same patient before disease onset), whereby an increase in the assayed level of KIT antigen compared to the control level of the KIT antigen is indicative of a KIT-mediated disease.

Methods for detection are known to one of skill in the art. For example, the anti-KIT antibody can be conjugated to a detectable molecule (e.g., as described in section 5.1.6), and the detectable molecule can be visualized using standard techniques (e.g., microscopy). Antibodies described herein can be used to assay KIT antigen levels in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as ELISA and the radioimmunoassay (RIA). Suitable antibody assay labels are known in the art and include enzyme labels, such as, glucose oxidase; (¹²¹In) radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin. In specific embodiments, diagnostic methods described herein involve using naked or unlabeled antibodies not conjugated to a detectable marker, and the naked or unlabeled antibodies are detected indirectly, e.g., by using a secondary antibody, which can be labeled.

In certain embodiments, high expression of KIT in a sample relative to a normal control sample (e.g., sample obtained from a healthy patient not suffering from a KIT-mediated disorder or disease) indicates that the patient is suffering from a KIT-mediated disorder or disease.

A method for diagnosing a patient with a KIT-mediated disorder or disease, such as cancer, in a sample obtained from a patient comprises contacting the sample with an anti-KIT antibody described herein and detecting the expression level of KIT in the sample. In certain embodiments, high expression of KIT in a sample relative to a normal control sample (e.g., sample obtained from a healthy patient not suffering from a KIT-mediated disorder or disease) indicates that the patient is suffering from a KIT-mediated disorder or disease.

In certain embodiments, a sample can be a tumor sample derived from, or comprising tumor cells from, a patient's tumor. Examples of tumor samples herein include, but are not limited to, tumor biopsies, circulating tumor cells, circulating plasma proteins, ascitic fluid, primary cell cultures or cell lines derived from tumors or exhibiting tumor-like properties, as well as preserved tumor samples, such as formalin-fixed, paraffin-embedded tumor samples or frozen tumor samples. In certain embodiments, a sample is a fixed tumor sample which has been histologically preserved using a fixative. In some embodiments, a sample is a formalin-fixed tumor sample which has been preserved using formaldehyde as the fixative. In certain embodiments, a sample is an embedded tumor sample which is surrounded by a firm and generally hard medium such as paraffin, wax, celloidin, or a resin. Embedding makes possible the cutting of thin sections for microscopic examination or for generation of tissue microarrays (TMAs). In particular embodiments, a sample is a paraffin-embedded tumor sample which is surrounded by a purified mixture of solid hydrocarbons derived from petroleum. In certain embodiments, a sample is a frozen tumor sample which is, or has been, frozen.

In certain aspects, a cancer or biological sample which displays KIT expression, amplification, or activation is one which, in a diagnostic test, expresses (including overexpresses) a KIT receptor, has amplified KIT gene, and/or otherwise demonstrates activation or phosphorylation of a KIT receptor.

Also provided herein is the detection and diagnosis of a KIT-mediated disease in a human. In one embodiment, diagnosis comprises: a) administering (for example, parenterally, subcutaneously, or intraperitoneally) to a subject an effective amount of a labeled antibody described herein (e.g., antibody Ab1 or Ab21, an antibody comprising the VL CDRs and/or VH CDRs of antibody Ab1 or Ab21, an antigen-binding fragment of any such antibody, or a conjugate comprising any such antibody or antigen-binding fragment); b) waiting for a time interval following the administering for permitting the labeled antibody to preferentially concentrate at sites in the subject where the KIT antigen is expressed (and for unbound labeled molecule to be cleared to background level); c) determining background level; and d) detecting the labeled antibody in the subject, such that detection of labeled antibody above the background level indicates that the subject has a KIT-mediated disease. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹Tc. The labeled antibody will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled antibody to preferentially concentrate at sites in the subject and for unbound labeled antibody to be cleared to background level is 6 to 48 hours or 6 to 24 hours or 6 to 12 hours. In another embodiment the time interval following administration is 5 to 20 days or 5 to 10 days.

In one embodiment, monitoring of a KIT-mediated disease is carried out by repeating the method for diagnosing the a KIT-mediated disease, for example, one month after initial diagnosis, six months after initial diagnosis, one year after initial diagnosis, etc.

Presence of the labeled molecule can be detected in the subject using methods known in the art for in vivo scanning. These methods depend upon the type of label used. Skilled artisans will be able to determine the appropriate method for detecting a particular label. Methods and devices that can be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

### 5.8 Other Methods

Provided herein are methods for inhibiting KIT activity in a cell expressing KIT comprising contacting the cell with an effective amount of an antibody described herein (e.g., antibody Ab1 or Ab21, an antibody comprising the VL CDRs and/or VH CDRs of antibody Ab1 or Ab21, an antigen-binding fragment of any such antibody, or a conjugate comprising any such antibody or antigen-binding fragment). Also provided herein are methods for inducing or enhancing apoptosis in a cell expressing KIT comprising contacting the cell with an effective amount of an antibody described herein (e.g., antibody Ab1 or Ab21, an antibody comprising the VL CDRs and/or VH CDRs of antibody Ab1 or Ab21, an antigen-binding fragment of any such antibody, or a conjugate comprising any such antibody or antigen-binding fragment). Also provided herein are methods for inducing or enhancing cell differentiation in a cell expressing KIT comprising contacting the cell with an effective amount of an antibody described herein (e.g., antibody Ab1 or Ab21, an antibody comprising the VL CDRs and/or VH CDRs of antibody Ab1 or Ab21, an antigen-binding fragment of any such antibody, or a conjugate comprising any such antibody or antigen-binding fragment). In certain aspects, the methods described herein can be performed in vitro or in vivo (e.g., by applying an antibody described herein to a specific area of the body, or by administering an antibody described herein to an individual).

KIT activity and, for example, the effect of an antibody on KIT activity can routinely be assessed using, e.g., cell-based assays such as those described herein.

Non-limiting examples of KIT activity which can be inhibited by the methods provided herein can include any activity of KIT known or described in the art, e.g., KIT receptor dimerization, KIT receptor phosphorylation (tyrosine phosphorylation), signaling downstream of the KIT receptor (e.g., Stat, AKT, MAPK, or Ras signaling), KIT ligand (e.g., SCF) induced transcriptional regulation (e.g., SCF-induced transcriptional activation of c-Myc), induction or enhancement of cell proliferation, or cell survival.

In certain embodiments, a method for inhibiting KIT activity in a cell expressing KIT comprises contacting the cell with an effective amount of an antibody described herein sufficient to inhibit or antagonize KIT activity by at least about 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein and/or known to one of skill in the art (e.g., ELISA). Non-limiting examples of KIT activity can include KIT receptor phosphorylation, KIT receptor signaling, KIT ligand (e.g., SCF) mediated cell proliferation, KIT ligand (e.g., SCF) mediated cell survival, and transcriptional activation of a KIT target gene (e.g., c-Myc).

In a particular embodiment, a method for inhibiting KIT activity in a cell expressing KIT comprises contacting the cell with an effective amount of an antibody described herein sufficient to inhibit or antagonize downstream KIT signaling, for example, signaling of a member of the Src family kinases, PI 3-kinases, or Ras-MAPK.

In another particular embodiment, a method for inhibiting KIT activity in a cell expressing KIT comprises contacting the cell with an effective amount of an antibody described herein sufficient to inhibit or antagonize downstream KIT signaling such as phosphorylation of MAPK, phosphorylation of AKT, or phosphorylation of Stat1, Stat3, or Stat5. Thus, in certain embodiments, a method for an inhibiting or antagonizing KIT activity in a cell expressing KIT comprises contacting the cell with an effective amount of an antibody described herein sufficient to inhibit or to reduce phosphorylation of MAPK (e.g., KIT ligand (e.g., SCF) induced phosphorylation of MAPK) by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art, e.g., Western blot or ELISA assay as described in section 6 or immunoblotting assay.

In certain embodiments, a method for inhibiting KIT activity in a cell expressing KIT comprises contacting the cell with an effective amount of an antibody described herein sufficient to inhibit or to reduce phosphorylation of AKT (e.g., KIT ligand (e.g., SCF) induced phosphorylation of AKT) by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art, e.g., Western blot or ELISA assay as described in section 6 or immunoblotting assay.

In particular embodiments, a method for inhibiting KIT activity in a cell expressing KIT comprises contacting the cell with an effective amount of an antibody described herein sufficient to inhibit or reduce phosphorylation of Stat3 (e.g., KIT ligand (e.g., SCF) induced phosphorylation of Stat3) by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art, e.g., Western blot or ELISA assay as described in section 6 or immunoblotting assay.

In particular embodiments, a method for inhibiting KIT activity in a cell expressing KIT comprises contacting the cell with an effective amount of an antibody described herein sufficient to inhibit or reduce phosphorylation of Stat1 or Stat5 (e.g., KIT ligand (e.g., SCF) induced phosphorylation) by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art, e.g., Western blot or ELISA assay as described in section 6 or immunoblotting assay.

In certain aspects, a method for inhibiting KIT activity in a cell expressing KIT comprises contacting the cell with an effective amount of an antibody described herein sufficient to inhibit proliferation of the cell. Cell proliferation assays are described in the art and can be readily carried out by one of skill in the art. For example, cell proliferation can be assayed by measuring Bromodeoxyuridine (BrdU) incorporation (see, e.g., Hoshino et al., 1986, Int. J. Cancer 38, 369; Campana et al., 1988, J. Immunol. Meth. 107:79) or (3H) thymidine incorporation (see, e.g., Blechman et al., Cell, 1995, 80:103-113; Chen, J., 1996, Oncogene 13:1395-403; Jeoung, J., 1995, J. Biol. Chem. 270:18367 73), by direct cell count at various time intervals (e.g., 12-hour or 24-hour intervals), or by detecting changes in transcription, translation or activity of known genes such as proto-oncogenes (e.g., fos, myc) or cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as ELISA, Western blotting or immunoprecipitation using antibodies, including commercially available antibodies. mRNA can be quantitated using methods that are well known and routine in the art, for example, using northern analysis, RNase protection, or polymerase chain reaction in connection with reverse transcription.

In specific embodiments, a method for inhibiting KIT activity in cells expressing KIT comprises contacting the cells with an effective amount of an antibody described herein sufficient to inhibit cell proliferation by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art (e.g., BrdU incorporation assay). In specific embodiments, a method for an inhibiting or antagonizing KIT activity in cells expressing KIT comprises contacting the cells with an effective amount of an antibody described herein sufficient to inhibit cell proliferation by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art (e.g., BrdU incorporation assay).

In certain aspects, a method provided herein for inhibiting KIT activity in a cell expressing KIT comprises contacting the cell with an effective amount of an antibody described herein sufficient to reduce or to inhibit survival of the cell. Cell survival assays are described in the art and can be readily carried out by one of skill in the art. For example, cell viability can be assessed by using trypan-blue staining or other cell death or viability markers known in the art. In a specific embodiment, the level of cellular ATP is measured to determined cell viability. In specific embodiments, cell viability is measured in three-day and seven-day periods using an assay standard in the art, such as the CellTiter-Glo Assay Kit (Promega) which measures levels of intracellular ATP. A reduction in cellular ATP is indicative of a cytotoxic effect. In another specific embodiment, cell viability can be measured in the neutral red uptake assay. In other embodiments, visual observation for morphological changes can include enlargement, granularity, cells with ragged edges, a filmy appearance, rounding, detachment from the surface of the well, or other changes. These changes are given a designation of T (100% toxic), PVH (partially toxic-very heavy-80%), PH (partially toxic-heavy-60%), P (partially toxic-40%), Ps (partially toxic-slight-20%), or 0 (no toxicity-0%), conforming to the degree of cytotoxicity seen. A 50% cell inhibitory (cytotoxic) concentration (IC₅₀) is determined by regression analysis of these data.

In specific embodiments, a method provided herein for inhibiting KIT activity in cells expressing KIT comprises contacting the cells with an effective amount of an antibody described herein sufficient to reduce or to inhibit survival of the cells by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art (e.g., trypan blue exclusion assay). In specific embodiments, a method provided herein for inhibiting KIT activity in cells expressing KIT comprises contacting the cells with an effective amount of an antibody described herein sufficient to reduce or to inhibit survival of the cells by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art (e.g., trypan blue assay).

In a specific embodiment, a method provided herein for inhibiting KIT activity in cells expressing KIT comprises contacting the cells with an effective amount of an antibody described herein sufficient to induce apoptosis (i.e., programmed cell death). Methods for detecting apoptosis are described in the art and can be readily carried out by one of skill in the art. For example, flow cytometry can be used to detect activated caspase 3, an apoptosis-mediating enzyme, in cells undergoing apoptosis, or Western blotting can be used to detect cleavage of poly(ADP-ribose) polymerase (PARP (see, e.g., Smolich et al., Blood, 2001, 97:1413-1421). Cleavage of PARP is an indicator of apoptosis. In specific embodiments, a method provided herein for an inhibiting or antagonizing KIT activity in cells expressing KIT comprises contacting the cells with an effective amount of an antibody described herein sufficient to induce or enhance apoptosis by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art (e.g., flow cytometry to detect activated caspases 3). In specific embodiments, antibodies a method provided herein for inhibiting KIT activity in cells expressing KIT comprises contacting the cells with an effective amount of an antibody described herein sufficient to induce or enhance apoptosis by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art (e.g., flow cytometry to detect activated caspase 3).

In a specific embodiment, a method provided herein for inhibiting KIT activity in a cell expressing KIT comprises contacting the cells with an effective amount of an antibody described herein sufficient to induce differentiation. Methods for detecting differentiation are described in the art and can be readily carried out by one of skill in the art. For example, flow cytometry can be used to detect expression of one or more differentiation markers, or the lack of expression of one or more undifferentiated markers, in a cell contacted with an antibody described herein. Similarly, Western blotting can also be used to detect differentiation markers. Suitable differentiation markers and undifferentiated markers have been described and are one of skill in the art.

In specific embodiments, a method provided herein for inhibiting KIT activity in cells expressing KIT comprises contacting the cells with an effective amount of an antibody described herein sufficient to induce differentiation by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art (e.g., flow cytometry). In specific embodiments, a method provided herein for inhibiting KIT activity in cells expressing KIT comprises contacting the cells with an effective amount of an antibody described herein sufficient to induce differentiation by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art (e.g., flow cytometry).

Non-limiting examples of cells which can be differentiated by the methods described herein include stem cells (e.g., embryonic stem cells, hematopoietic stem cells) and progenitor cells. Exemplary hematopoietic stem cell markers include CD38, CD34, CD59, CD133, Sca-1, and ABCG2. Non-limiting examples of neural stem cell markers include Nestin, PSA-NCAM, p75 Neurotrophin R, and Vimentin. Other non-limiting examples of stem cell markers include, Oct4, Sox2, Klf4, LIN28, Nanog, SSEA-3, SSEA-4, Notch, and Wnt.

### 6. EXAMPLES

The examples in this section (i.e., section 6) are offered by way of illustration, and not by way of limitation.

Fab antibodies were generated from a human antibody phage library (Dyax Corporation, Cambridge, Mass.) by biopanning against the D4/D5 region of human KIT (see FIG. 2, in particular FIG. 2A depicting the recombinant antigen (containing the D4/D5 region of human KIT) used to screen the phage library). Over 300 phage clones were isolated. The variable regions of selected phage clones were subcloned into expression vectors for recombinant expression in mammalian host cells.

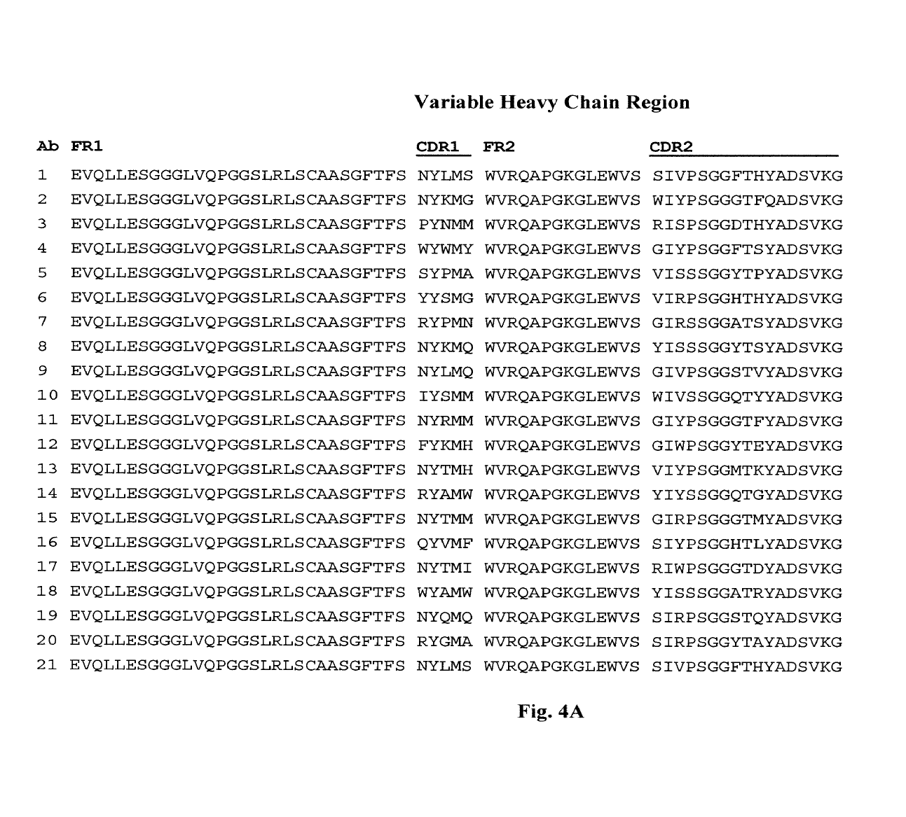

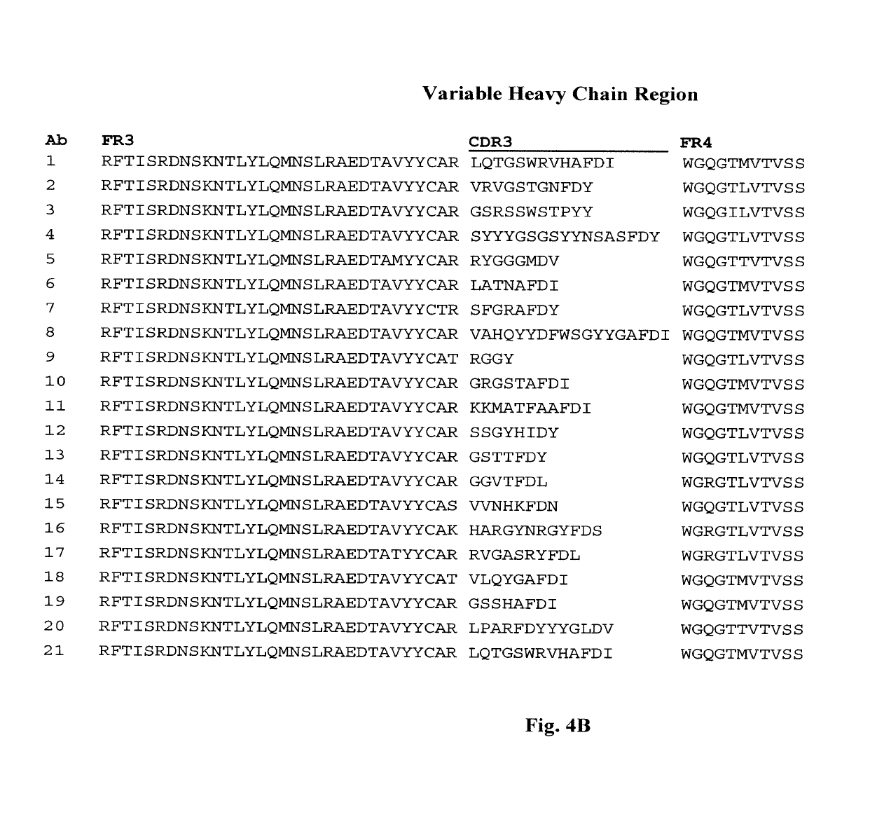

These Fabs were screened further for binding activity by solid phase ELISA directed against the D4/D5 region of KIT and for blocking activity by cell-based phospho-Kit assays. These experiments and their results are described in further details below. FIGS. 3A-B show the amino acid sequence of the VL domains of 20 selected Fab antibodies. FIGS. 4A-B show the amino acid sequence of VH domains of 20 selected Fab antibodies. FIGS. 5A-5T show the corresponding VL domain and VH domain of each of the 20 selected Fab antibodies, designated herein as Ab1-Ab20.

### 6.1 Example 1

### Screening Antibodies for Binding Activity by Solid Phase ELISA Directed Against the D4/D5 Region of KIT

The binding activity of Fab antibodies selected and generated based on a human antibody phage library (Dyax Corporation, Cambridge, Mass.) panned against the D4/D5 region of the human KIT extracellular region were characterized by solid phase ELISA. Fab antibodies designated Ab1, Ab2, Ab3, Ab4, Ab5, Ab6, Ab7, Ab8, Ab9, Ab10, Ab11, Ab12, Ab13, Ab14, Ab15, Ab16, Ab17, Ab18, Ab19, and Ab20 were tested.

Materials:
Recombinant antigen: Recombinant IG domain four and five of the extracellular region of KIT
Assay plates Nunc MaxiSorp mico titer plates 456537
plate sealers Fisher 353073
polypropylene dilution plates
TBS-T: 50 mM Tris pH 7.4, 150 mM NaCl, 0.1% Tween 20
TBS: 50 mM Tris pH 7.4, 150 mM NaCl
Blocking solution: 1% bovine serum albumin (BSA) in TBS
Dilution buffer: 1% BSA in TBS-T
Detection antibody solution: Pierce goat ant-human F(ab′)2 specific conjugated with horseradish peroxidase (Thermo scientific) 31414
Detection Substrate: TMB Substrate kit (Thermo scientific) #34021
Plate Washer: nunc immunowash 470176
ELISA plate reader: BioTek Synergy HT, Software Gen5 1.08

Coating plates with antigen: Recombinant antigen containing the D4/D5 region of the KIT extracellular Ig-like region (see FIG. 2A) were absorbed to 96-well microtiter plates. 10 μg of recombinant antigen (i.e., D4/D5 region of KIT) were diluted into 10 mL of borate buffer, and 100 μL of the antigen solution were added to each well of a 96 well plate. The 96 well plate was covered with a plate sealer, was incubated at 4° C. overnight, and then stored at −80° C. until the day of the assay.

Preparation of antibody sample with serial dilution: 100 μL of dilution buffer were added to columns 1 and 7 of a 96 well plate, and 50 μL of dilution buffer were added to all the remaining wells. 4 μL of the test antibody were added to the first well containing 100 μL dilution buffer, mixed by pipetting, and 50 μL from the first well were removed and added into the second well and mixed. This dilution step was repeated across the row until the dilution step was repeated for a total of six dilutions. This dilution process was repeated for each sample.

ELISA: The plate with the absorbed antigen was removed from the −80° C. freezer and was allowed to thaw at room temperature. The borate buffer was removed by flicking the plate into the sink and blotting the plate dry. 200 μL of the blocking buffer were added to each well and allowed to incubate at room temperature for one hour. The blocking buffer was removed, again by flicking the plate into the sink. Then, the diluted solution of test antibodies and controls (were added to the plate in a volume of 50 μL and incubated at room temperature for one hour. The antibody solutions were removed, and the plate was washed four times with 300 μL of wash buffer with five minute incubations. After the last wash, the plate was blotted dry. The diluted secondary antibody solution was added to each well in a volume of 100 μL and allowed to incubate for one hour at room temperature. The diluted secondary antibody solution was removed, and the plate was washed four times with 300 μL of wash buffer with five minute incubations. The plate was then blotted dry, and freshly mixed substrate solution was added to each well in a volume of 100 μL and was allowed to incubate at room temperature for 30 minutes. Subsequently, 100 μL of 2NH₂SO₄ were added to each well and immediately read on the plate reader. An irrelevant antibody served as the negative control, and an anti-KIT antibody against the D4 and/or D5 domain of the extracellular region of KIT served as the positive control. OD values for each sample were obtained at a wavelength of 450 nm.

Data analysis using Graph Pad Prism and Excel: Concentrations in ng/mL units were converted to μM based on the molecular weight of a Fab molecule (50 kDa) and of an IgG molecule (150 kDa). The OD values from the plate reader for the samples were exported into Graph Pad and the sample concentrations were transformed into log(X) and subjected to nonlinear regression (curve fit), then sigmoidal dose-response (variable slope) to obtain the concentration of antibodies at 50% binding to antigen.

At least a number of the 20 Fab antibodies (Ab1, Ab2, and Ab5-Ab12) tested exhibited a concentration (nM) at 50% binding to KIT antigen of approximately less than or equal to 300 nM.

### 6.2 Example 2

### Determination of Antibody Binding Specificity for the D4 Domain, D5 Domain, and/or D4/D5 Region of Human KIT by Solid Phase ELISA

To further characterize the antigen binding specificity of the Fab antibodies, solid phase ELISA was carried out using the following KIT polypeptides: the D4 domain, the D5 domain, and the D4/D5 region of human KIT (see FIGS. 2A-2C).

Solid phase ELISAs were carried out essentially as described in section 6.2, with the following modifications.

ELISA plates were coated with either (i) recombinant polypeptide containing the D4/D5 region of the KIT extracellular Ig-like region (see FIG. 2A), (ii) recombinant polypeptide containing the D4 domain of the KIT extracellular Ig-like region (see FIG. 2B), or (iii) recombinant polypeptide containing the D5 domain of the KIT extracellular Ig-like region (see FIG. 2C).

As described above in section 6.1 the following steps were carried out, antibody samples were added to the antigen-coated plates, then the secondary antibody solution was added, contacted with substrate solution prior to data collection on the plate reader. OD values for each sample were obtained at a wavelength of 450 nm.

As described above in section 6.1, data analysis was carried out using Graph Pad Prism and Excel. Antibodies Ab1, Ab2, Ab3, Ab13, Ab17, and Ab20 bound to the D4 domain of human KIT (see FIG. 2B for an illustration of the D4 domain polypeptide which was used to confirm the binding specificity); antibody Ab19 bound to the D5 domain of human KIT (see FIG. 2C for an illustration of the D5 domain polypeptide which was used to confirm the binding specificity); and antibodies Ab4, Ab14, Ab15, Ab16, and Ab18 bound to a human KIT fragment containing amino acid residues V308 to H515, including the D4/D5 region and the hinge region between the D4 and D5 domains, but does not bind to a human KIT fragment containing only the D4 domain or D5 domain individually (see FIG. 2A for an illustration of the D4/D5 polypeptide which was used to confirm the binding specificity).

### 6.3 Example 3

### Screening Antibodies Directed Against KIT for Blocking Activity by Cell-Based Phospho-KIT Assay

To further characterize the activity of Fab antibodies on KIT activity, specifically, tyrosine phosphorylation of the cytoplasmic domain of KIT, cell-based phosphor-KIT assays were carried out as follows.

Materials:
CHO stably transfected with a plasmid encoding full-length human KIT (see FIG. 1), which was cloned from a human ovary cDNA library (OriGene, Rockville, Md.)
Complete cell culture media (see Table 5)
Starving media: Cell culture media described in Table 5 without FBS
Trypsin-EDTA (Cellgro 25-050-CI)
PBS (GIBCO 10010-023)
24-well cell culture plates BD Falcon (353226)
ELISA plates (Nunc, #436110)
SCF solution: rhSCF (RD Systems 255-SC/CF); final concentration 30 ng/mL
Lysis buffer: 50 mM Tris pH 7.4, 150 mM NaCl, 1 mM EDTA, 1% Triton X-100, protease inhibitor cocktail tables EDTA free (Roche Diagnostics 04693132001), 1 mM NaVO₄
TBS-T: 50 mM Tris pH 7.4, 150 mM NaCl, 0.1% Tween 20
Blocking solution: 5% bovine serum albumin (BSA) in TBS-T
Dilution buffer: 1% BSA in TBS-T containing 1 mM NaVO₄
Detection antibody solution: anti-phospho-tyrosine antibody conjugated with horse radish peroxidase (Millipore, 4G10); dilution factor 1:500
Capture antibody: anti-CD117 Ab3 from Thermo Scientific (MS-289-PABX)
Cell incubator: Thermo Scientific, HeraCell 150i CO2 incubator
Cell Counter: Invitrogen Countess™ C10227
ELISA plate reader: BioTek Synergy HT, Software Gen5 1.08

TABLE 5
Cell Culture Media

| Cell line | CHO KIT transfected |
|---|---|
| Basic medium | HyClone Ham's F12 SH |
| | 30026.01 |
| Penicillin/Streptavidin | 50 IU/mL penicillin |
| (Cellgro 30-001-CI) | 50 ug/mL streptavidin |
| Glutamin (Gibco 35050) | 2 mM |
| Hygromycin B (Invitrogen | 500 ug/mL |
| 10687-010) | |

Passaging of CHO/KIT cells: Confluent cells were washed once with sterile PBS, incubated with 0.25% Trpsin-EDTA at room temperature until cells detach from the plastic tissue culture plates. Complete culture medium, which contains FBS, were added to the plate to end the tryptic digestion.

Counting Cells: 10 μL of cell suspension were mixed with 10 μL of 0.4% trypan blue. 10 μL of this mixture were pipetted into a cell counting chamber (Invitrogen), and the cells were counted. 200,000 cells were transferred into each well of a 24-well cell culture plate, and the cells were cultivated in complete medium (Table 5) for 24 hours under normal cell culture conditions (i.e., humidified 95% air and 5% CO2 atmosphere at 37° C.).

Cell Treatment: After the cells were plated in the 24-well plates and cultured overnight, the medium was removed, and the cell monolayer was washed once with PBS. The cells were then cultured for 24 hours in starvation medium under normal cell culture conditions. Then the cells were treated with the appropriate Fab solutions or control antibody solutions for 2 hours under normal cell culture conditions. The final concentration for the Fab solution was 100 nM (5 μg/mL) or less. Subsequently, SCF solution at a final concentration of 30 ng/mL were added to the cells pretreated with Fabs or control antibodies for 10 minutes under normal cell culture conditions.

Controls:
Negative controls: starved, untreated and non-stimulated cells; two Fab samples that were made against an unrelated target
Positive control: starved, untreated and SCF-stimulated cells
Drug controls: starved, treated with 1 uM Gleevec and SCF-stimulated cells
Antibody control: starved, treated with 100 nM blocking antibody purified anti-human KIT (mouse) binds to SCF binding site (BioLegend A3C6E2)

Preparation of cell lysates: Afer stimulation, the 24-well plate was placed on ice immediately, the cells were washed once with cold PBS, and the cells were lysed with 100 μL of cold lysate buffer.

Preparation of 96-well ELISA plate with capture antibody: 5 μL of capture antibody were diluted in 10 mL 50 mM Borate buffer, and the capture antibody solution (100 μL or 50 ng/well) was added to each well of the 96-well ELISA plate. The 96-well plate was incubated at room temperature for 5-6 hours or overnight at 4° C. The capture antibody solution was removed prior to the blocking step. Blocking was carried out by adding 100 μL of blocking solution to each well and allowed to incubate at room temperature for 1 hour. The blocking solution was removed, the wells were washed once with dilution buffer, and 50 μL of dilution buffer were added to each well.

Phospho-Kit assay: 50 μL of the cell lysates of each sample from the 24-well plate were transferred into the prepared 96-well plate containing 50 μL dilution buffer in each well, and the 96-well plate was incubated overnight at 4° C. Following the overnight incubation, the supernatant was removed, and the plate was washed 3 times (5 minute incubation each time) with TBS-T. 100 μL of detection antibody dilution were added to each well and incubated for 1 hour at room temperature in the dark, the plate was washed 3 times with TBS-T, washed once with TBS, and the TBS was removed. The “SuperSignal West Dura Extended Duration Substrate” reagents 1:1 (Thermo Scientific) were mixed, and 100 μL of the mix were added to each well.

Luminescence was detected in the ELISA plate reader using the Gen5 protocol “Luminescence Glow” and the data was analyzed using Microsoft Excel. At least a number of the Fab antibodies tested, (Ab1, Ab2, and Ab5-Ab12) inhibited KIT phosphorylation in these phosphor-KIT assays.

Moreover, Fab antibody Ab1 was converted into a full IgG antibody containing human IgG constant regions using the Dyax (Cambridge, Mass.) technology platform. In particular, the polynucleotides encoding variable light chain region and variable heavy chain region of Fab antibody Ab1 were cloned into the Dyax pR rapid reformatting vector. The IgG version of Fab antibody Ab1 immunospecifically bound to the D4/D5 region of human KIT in binding assays, for example, a concentration at 50% binding of approximately 7 pM. The IgG version of Fab antibody Ab1 also exhibited robust inhibition of KIT phosphorylation (e.g., IC₅₀ of approximately 2.5 nM) in the phospho-KIT assays.

### 6.4 Example 4

### Screening of Anti-KIT Antibodies for Blocking Activity by Cell-Based Phospho-AKT Assay

Anti-KIT antibodies are assayed for the ability to inhibit or block AKT phosphorylation, which is a downstream signaling event of KIT signaling. The assay is carried out as described in section 6.3 except for a few minor modifications, primarily, use of an anti-AKT antibody as a capture antibody which is immobilized on the ELISA plates instead of an anti-KIT antibody as a capture antibody. An anti-AKT antibody which can be used as a capture antibody which is immobilized on the ELISA plates is Product No. PA1-37043 or PA1-32158 from ThermoScientific (Rockford, Ill.).

### 6.5 Example 5

### Xenograft Mouse Model Study of Anti-KIT Antibodies in Treating Cancer

The anti-tumor effects of anti-KIT antibodies described herein are confirmed using xenograft mouse models of human tumors (see, e.g., Fernandez et al., J. Clin. Invest., 2007, 117(12): 4044-4054). Xenograft mouse models of gastrointestinal stromal tumor cancer and myelogenous leukemia are described below.

### Xenograft Mouse Model of Gastrointestional Stromal Tumor (GIST) Cancer

Mouse models of GIST have been desecribed, for example, see, Fernéndez et al, J. Clin. Invest., 2007, 117(12): 4044-4054. Briefly, Female C.B-17/IcrHsd-PrkdcSCID mice are purchased from Harlan Sprague Dawley Inc. and housed in facilities approved by and in accordance with the American Association for Assessment and Accreditation of Laboratory Animal Care, the United States Department of Agriculture, the United States Department of Health and Human Services, and the NIH. Mice are used according to institutional guidelines when they are 8-12 weeks of age. GIST882 cells, which are immortal (gastrointestional stromal tumor) GIST cells that possess a homozygous exon 13 missense mutation (i.e., K642E) in KIT (see Tuveson et al., Oncogene, 2001, 20: 5054-5058), are harvested from subconfluent cultures by a brief exposure to 0.25% trypsin-EDTA (Invitrogen). Trypsinization is stopped with medium containing 10% FBS. The cells are then washed twice in serum-free medium and resuspended in serum-free HBSS (Invitrogen). Single-cell suspensions with greater than 95% viability, as determined by Trypan blue exclusion, are used for the injections. To produce tumors, 1×10⁵ to 1×10⁷ GIST882 cells, for example 6×10⁶ GIST882 cells, per 100 μl are injected subcutaneously into the unilateral flank of each SCID mouse. Five to ten mice per group in the vehicle and anti-KIT antibody groups are used. Once tumors are palpable (11 weeks from injection), mice are started on therapy with daily, weekly, or bi-weekly intraperitoneal injections of normal saline (vehicle) or anti-KIT antibodies. Treatment is continued for 6 weeks, with weekly 2-dimensional measurements of tumor size. All mice are sacrificed when the tumor size approached 1.5 cm in the control group. Tumors are collected, are fixed in formalin, and are analyzed by H&E staining. Representative images are taken from each tumor using a light microscope at ×40 and x100 magnification.

A graft of tumor size or volume of each mice plotted against time (e.g., days or weeks) after tumor injection is generated to ascertain the effect of the anti-KIT antibodies on tumor growth in the mice relative to the vehicle negative control.

### Xenograft Mouse Model of Myelogenous Leukemia

To study the effects of anti-KIT antibodies on leukemia, a xenograft mouse model using the cell line K562 (human immortalized myelogenous leukemia line), HEL, or HL60, is established essentially as described above, except that tumor cells are injected into the mice instead of GIST882 cells. In particular, tumor cells are collected from subconfluent suspensions, as described above. To produce tumors, 1×10⁵ to 1×10⁷, for example 10×10⁶, tumor cells per 100 μl are injected into each SCID mouse. The mice are then randomized into the following groups (n=7 per group): (a) normal saline daily; and (b) anti-KIT antibodies. The mice are started on therapy (e.g., at day 0, 7, or 14 or when tumors are detectable) with daily, weekly, or bi-weekly intraperitoneal injections of normal saline (vehicle) or anti-KIT antibodies. Treatment is continued for 6 weeks, with weekly 2-dimensional measurements of tumor size. Imaging methods for detecting tumor size can also be used, e.g., MRI. Tumors are measured weekly during treatment and at necropsy.

A graph of tumor size or volume of each mice plotted against time (e.g., days or weeks) after tumor injection is generated to ascertain the effect of the anti-KIT antibodies on tumor growth in the mice relative to the vehicle negative control.

Similar experiments are carried out with the HEL cell line, which is a human erythroleukemia cell line.

### Xenograft Mouse Model of Small Cell Lung Cancer

To study the effects of anti-KIT antibodies on leukemia, a xenograft mouse model using the H526 cell line (human small cell lung carcinoma cell line) is established essentially as described above, except that H526 cells are injected into the mice instead of GIST882 cells. In particular, H526 tumor cells are collected from subconfluent suspensions, as described above. To produce tumors, 1×10⁵ to 1×10⁷, for example 10×10⁶, H526 cells per 100 μl are injected into each SCID mouse. The mice are then randomized into the following groups (n=7 per group): (a) normal saline daily; and (b) anti-KIT antibodies. The mice are started on therapy (e.g., at day 0, 7, or 14 or when tumors are detectable) with daily, weekly, or bi-weekly intraperitoneal injections of normal saline (vehicle) or anti-KIT antibodies. Treatment is continued for 6 weeks, with weekly 2-dimensional measurements of tumor size. Imaging methods for detecting tumor size can also be used, e.g., MRI. Tumors are measured weekly during treatment and at necropsy.

A graph of tumor size or volume of each mice plotted against time (e.g., days or weeks) after tumor injection is generated to ascertain the effect of the anti-KIT antibodies on tumor growth in the mice relative to the vehicle negative control.

### 6.6 Example 6

### Competitive Binding Assays

Competitive binding assays are carried out with an antibody sample and any of antibodies Ab1-Ab21, Ab167, and Ab63 (see FIGS. 5A-5W) as described below. For this example, Ab1 (FIG. 5A) is utilized. In particular, the antibody sample is incubated with biotin-conjugated Ab1 antibody in wells of 96-well plates coated with a recombinant KIT polypeptide containing the D4/D5 region depicted in FIG. 2A. The biotin-conjugated Ab1 antibody bound to the D4/D5 region of KIT in the presence and absence of the antibody sample is detected by streptavidin-conjugated horseradish peroxidase.

Coating plates with antigen: Recombinant antigen containing the D4/D5 region of the KIT extracellular Ig-like region (see FIG. 2A) is absorbed to 96-well microtiter plates. 10 μg of recombinant antigen (i.e., D4/D5 region of human KIT) are diluted into 10 mL of borate buffer, and 100 μL of the antigen solution are added to each well of a 96 well plate. The 96 well plate is covered with a plate sealer, is incubated at 4° C. overnight, and then is stored at −80° C. until the day of the assay.

Six, 10 fold dilutions of the antibody sample are prepared. The plate with the absorbed antigen, is incubated with 200 μL of the blocking buffer at room temperature for one hour, prior to incubation with the antibody sample and biotin-conjugated Ab1 antibody. The control wells are incubated with buffer, antibody sample alone, or biotin-conjugated Ab1 antibody along at the concentrations tested. The plates are washed, and streptavidin-conjugated to horseradish peroxidase (BD Biosciences, San Jose, Calif.) is added to the wells to detect biotin-conjugated Ab1 antibody. The plates are washed freshly mixed substrate solution is added to each well, and the plates are processed similarly as described above for data collection on a plate reader. Data analysis is carried out using Graph Pad Prism and Excel as described above.

A reduced amount of Ab1 binding relative to control samples indicates that the antibody in the antibody sample competitively blocks binding of Ab1 to the D4/D5 region of KIT. The results can show that antibody Ab21 competitively blocks binding of Ab1 to the D4/D5 region of KIT.

### 6.7 Example 7

### Binding Assays for Human Monoclonal Ab1 and Ab21 Antibodies

Antibody Ab21 was derived from antibody Ab1 and was generated by substituting the N residue in VL CDR3 to an S residue to eliminate a glycosylation site. Both antibodies Ab1 and Ab21 were cloned into a full-length human IgG1, and the binding activities of these antibodies, Ab1 and Ab21 full-length human IgG1 antibodies, were tested by solid phase ELISA for binding to the D4/D5 region of the extracellular domain of human KIT.

Materials and equipment used in the binding assays include:

1. Recombinant D4/D5 region of human KIT
2. Assay plates Nunc MaxiSorp microtiter plates #456537
3. Plate sealer's Fisher #353073
4. Coat buffer: BupH Borate buffer pack (Thermo #28384—50 mM borate pH 8.5—filter after making)
5. TBS-T (wash buffer): 50 mM Tris pH 7.4, 150 mM NaCl, 0.1% Tween-20

### 6. TBS: 50 mM Tris pH 7.4, 150 mM NaCl

7. Blocking solution: 1% bovine serum albumin (BSA) in TBS
8. Dilution buffer: 1% BSA in TBS 0.05% Tween-20
9. Detection antibody solution: Pierce goat anti-human F(ab′)2 or IgG (mouse or human) specific conjugated with HRP (for Fabs: Thermo scientific #31414; for IgGs: Bethyl goat anti-human IgG Fc HRP #A80-104P-71)
10. Detection Substrate: TMB Substrate kit (Thermo scientific #34021)
11. Stop solution: 2.5 N sulfuric acid (BDH922-1)

The binding assays with antibodies Ab1 and Ab21 were carried out as follows. First, the plates were coated with antigen (i.e., recombinant D4/D5 region of human KIT). Specifically, 5 μg of the antigen was diluted into 10 ml of borate buffer and 100 μl was added to each well of a 96 well plate; the plate was covered with a plate sealer (50 ng/well for IgG) and was incubated overnight at 4° C. (or 3 hr at 37° C.), then it was frozen at −80° C. until the day of the assay.

Serial antibody dilutions were prepared as follows. A starting antibody concentration of 300 nM (or lower depending on sample) was diluted down 1:3 in dilution buffer. Specifically, 200 μl of the starting concentration (top row of dilution plate) was prepared, and 75 μl was diluted into 150 μl in the row below. The dilution was mixed, and the dilution step was repeated down the plate. From the dilution plate, 100 μl was transferred to the assay plate.

The binding assays were carried out as follows. The plate coated with antigen (i.e., recombinant D4/D5 region of KIT) were removed from the −80° C. freezer and were allowed to warm to room temperature. The borate buffer was removed by flicking the plate into the sink and blotting it dry. 200 μL of the blocking buffer (1% BSA/TBS) was added and incubated at room temperature for one hour. The blocking buffer was then removed by flicking the plate into the sink, and blotting dry. Then, the serially diluted antibodies and controls were added to the plate in a volume of 100 μL, and the plate was sealed and incubated at room temperature for one hour.

Subsequently, the plate contents were removed, and the plate was washed four times (one quick and 3×5 minutes) with 300 μL of wash buffer. At the end of the last wash, the plate was blotted dry.

To start the detection steps, 100 μL of the diluted secondary antibody (anti-mouse or -human; 1:10,000) was added to each well of the 96 well plate, and the plate was sealed and incubated for one hour. Then the plate was washed as described above. Subsequently, 100 μL of freshly mixed substrate was added to each well and incubated at room temperature for up to 30 minutes. The plate was monitored for development, and the development step was stopped with acid after blue color came up. Specifically, 100 μL of 2.5 NH₂SO₄ was added to stop development and immediately read on the plate reader (450 nm).

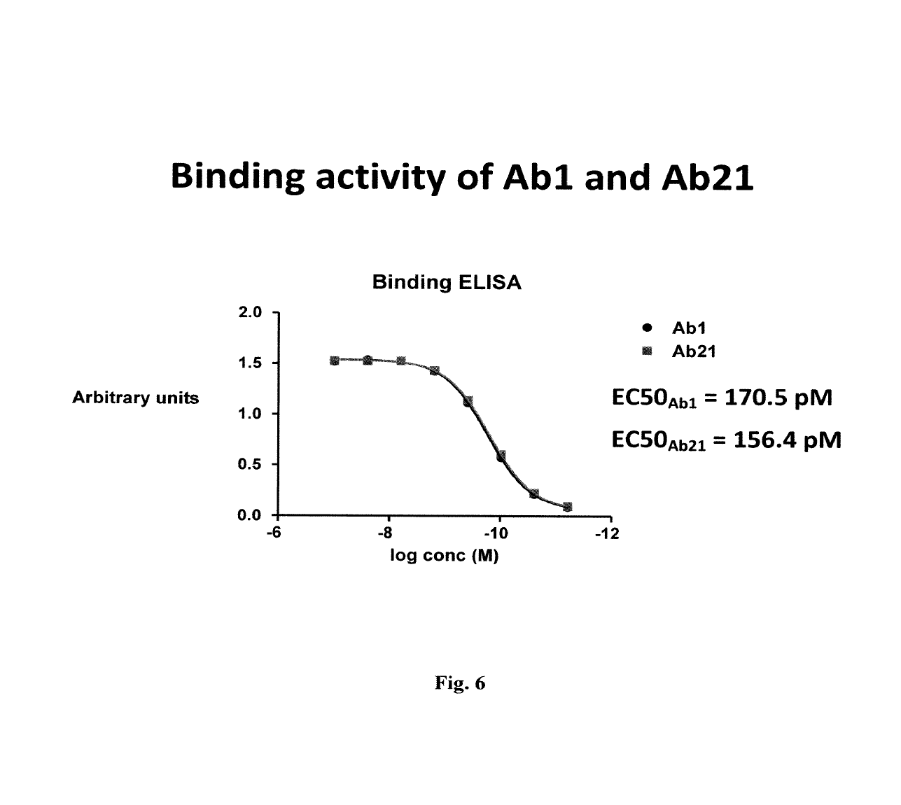

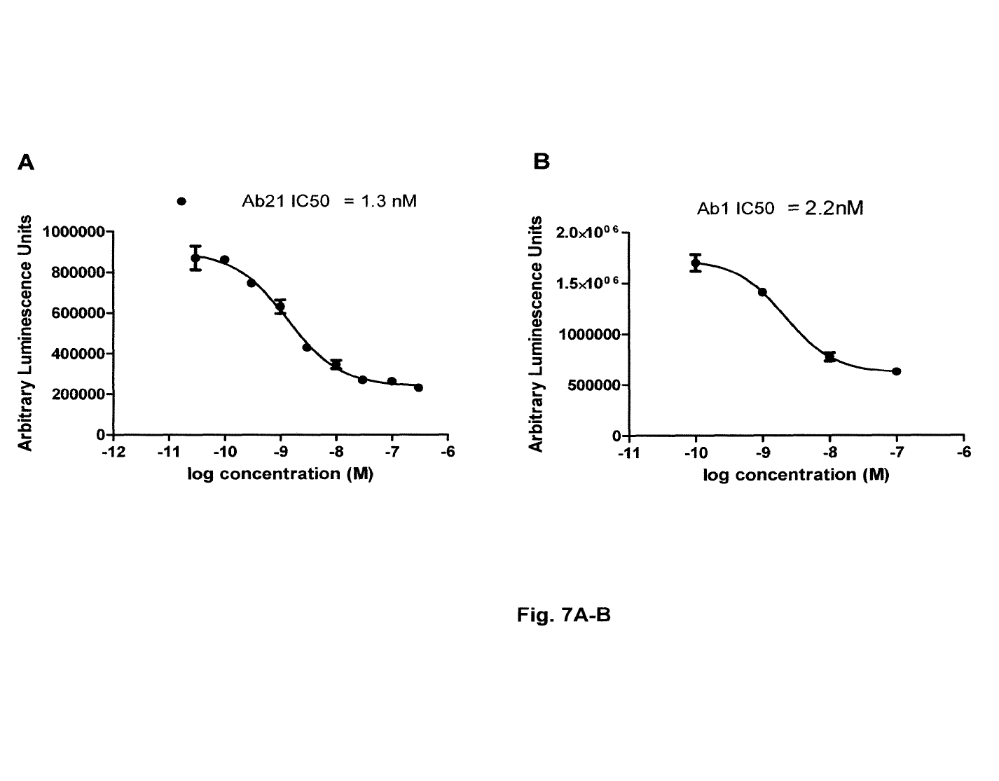

The binding data was plotted on a graph (antibody concentration versus arbitrary units) as presented in FIG. 6. The effective concentration at 50% binding (EC₅₀) for each antibody was calculated. The EC₅₀ for antibodies Ab1 and Ab21 were calculated to be 170.5 pM and 156.4 pM, respectively. Thus, the binding data indicate that both antibody Ab1 and antibody Ab21, which contains a VL CDR3 with an N to S substitution to eliminate a glycosylation site, bind with high affinity to the D4/D5 region of human KIT. Thus, these results, coupled with the amino acid sequences of antibody Ab1 and Ab21, indicate that Ab21 can compete with antibody Ab1 for binding to the D4/D5 region of human KIT, and that Ab21 binds to the same epitope as the epitope of Ab1.

### 6.8 Example 8

### Cell-Based KIT Phosphorylation Assays with Antibodies Ab1 and Ab21

Both full-length human IgG1 antibodies Ab1 and Ab21 were tested by cell-based phosphorylation assay for their inhibitory effects on KIT activity. Specifically, tyrosine phosphorylation of the cytoplasmic domain of KIT was measured by cell-based phosphor-KIT assays, which were carried out essentially as described in Section 6.3.

The results from the cell-based phosphorylation assay are presented in FIGS. 7A (antibody Ab21) and 7B (antibody Ab1). The 50% inhibition concentration (IC₅₀) of the full-length human IgG1 antibody Ab1 was calculated to be 2.2 nM (FIG. 7B), and the IC₅₀ of the full-length human IgG1 antibody Ab21 was calculated to be 1.3 nM (FIG. 7A).

### 6.9 Example 9

### Inhibition of KIT Phosphorylation by Ab21 in the Small Cell Lung Cancer Cell Line H526 and in CHO Cells Overexpressing KIT

Detection of the ability of full-length human IgG1 antibody Ab21 to inhibit tyrosine phosphorylation of the cytoplasmic domain of KIT was carried out by Western Immunoblotting assays.

The materials and reagents for the Western Immunoblotting assays are described as follows. Proteins of all samples were electrophoretically separated using the NuPAGE® system developed by Invitrogen Corp. (Carlsbad, Calif.). Other materials include:
Bradford protein quantification assay (BioRad),
Blocking solution: Tris buffered saline, 0.1% Tween-20, 5% fat-free milk powder;
Dilution buffer: Tris buffered saline, 0.1% Tween-20, 1% bovine serum albumin; and
Developping reagents: SuperSignal West Dura Luminol/Enhancer solution and SuperSignal West Dura Stable Peroxide Buffer (Thermo Scientific)Procedure.

H526 cells were treated with KIT targeting tyrosine kinase inhibitors or antibody Ab21 at various concentrations, stimulated with SCF (0.1, 1, 10, and 100 ng/mL), and lysed as described previously in the protocol for the cell-based assay (sections 6.3 and 6.8). Protein concentration was determined using the Bradford quantification assay. Equal amounts of protein were loaded onto precasted NuPAGE® Novex® Bis-Tris Mini gels (Invitrogen Corp. (Carlsbad, Calif.)).

The proteins were separated by gel electrophoresis at 175 Volt for approximately 60 minutes. Then the proteins were transferred from the gel onto nitrocellulose membranes (Invitrogen Corp. (Carlsbad, Calif.)) for 1-2 hours at 25V in a transfer buffer solution containing 20% methanol, using the Trans-Blot® SD semi-dry transfer cell system from BioRad (Hercules, Calif.). The membranes were blocked in blocking solution for at least 20 mins. Subsequently, the membranes were incubated with primary antibody diluted in dilution buffer for example anti-KIT, anti-phospho-KIT antibody (Cell signaling), and anti-phospho-Erk-1,2 antibody, over night at 4° C. Anti-beta-tubulin antibody was used an a loading control. The membranes were then incubated in secondary antibody, for example anti-rabbit IgG conjugated with horse radish peroxidase for 60 min. at room temperature. The membranes were then incubated in developing reagents. Chemiluminescence was detected by the camera system Molecular Imager® ChemoDoc XRS+ from BioRad (Hercules, Calif.).

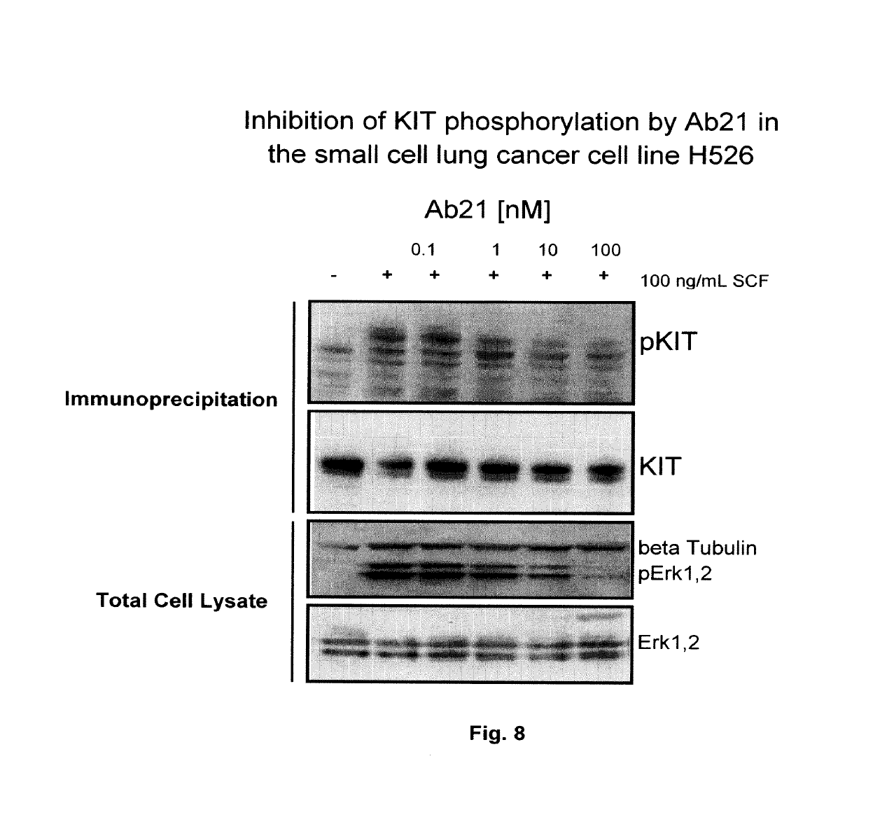

The Western blot depicted in FIG. 8 shows that SCF induces phosphorylation of KIT in H526 cells, and Ab21 inhibits such phosphorylation in a dose dependent manner Phosphorylation of Erk-1,2 is similarly affected.

The same Western Immunoblotting assay was carried out with CHO cells overexpressing full-length human KIT (CHO/KIT WT), and the results are depicted in FIG. 8. Briefly CHO cells were engineered to express exogenous full-length human KIT, and the cells were cultured in the presence and absence of SCF, and with or without Ab21. The CHO/KIT WT cells were harvested and cell lysates were obtained and processed for Western Immunoblotting analysis as described above.

Immunoprecipitation samples were processed as follows. Briefly, cells were cultured to 80% confluency in 10 cm-dishes, starved over night, and then treated for 2 hours at 37° C. with antibody Ab21 at the concentrations 0.1, 1, 10 or 100 nM. The cells were then stimulated with 100 ng/mL SCF for 10 minutes at 37° C. The dishes were then put on ice. The cells were washed with ice cold PBS, and were lysed in 400 uL/10 cm-dish with ice-cold lysis buffer (50 mM Tris pH 7.4, 150 mM NaCl, 1 mM EDTA, 1% Triton X-100, protease inhibitor cocktail tables EDTA free (Roche Diagnostics 04693132001), 1 mM NaVO₄). The lysates were transferred into tubes using needle and syringe; and passed through the cold needle three time for homogenization. The lysates were entrifuged at maximal speed for 20 minutes at 4° C., and the cleared supernatants were transferred into fresh cold tubes and thepellets were discarded. The protein concentration of the cleared lysated was determined by Bradford assay.

One mg protein of each sample at a concentration of 1-2 ug/uL were incubated with 4 ug of a commercial anti KIT antibody (Thermo Scientific MS-289-PABX) under rotation at 4° C. overnight. The next day, protein G agarose beads (Santa Cruz) were equiliberated, and 20 uL bead volume of the protein G agarose beads were added to each sample. The samples were placed on a rotating platform and were incubated with the beads at 4° C. for 2 hours. Then, the samples were centrifuged, and the superatants were aspirated. The pellets containing the protein G agarose beads and immunoprecipitated proteins were washed 3 times with ice cold lysate buffer. Twenty uL of NuPAGE® sample buffer (Invitrogen) were added to the beads and heated at 75° C. for 15 minutes. The entire sample volume was loaded onto an SDS-PAGE gel separated by electrophoresis. The gel then was processed as described in the western immune blotting protocol. Briefly, after the proteins on the gel were separated, they were transferred to a membrane, and the membrane was blocked prior to application of anti phospho-tyrosine antibody (R&D HAM1676). The membrane was then stripped with acidic buffer and reprobed with anti-KIT antibody (Cell signaling D13A2).

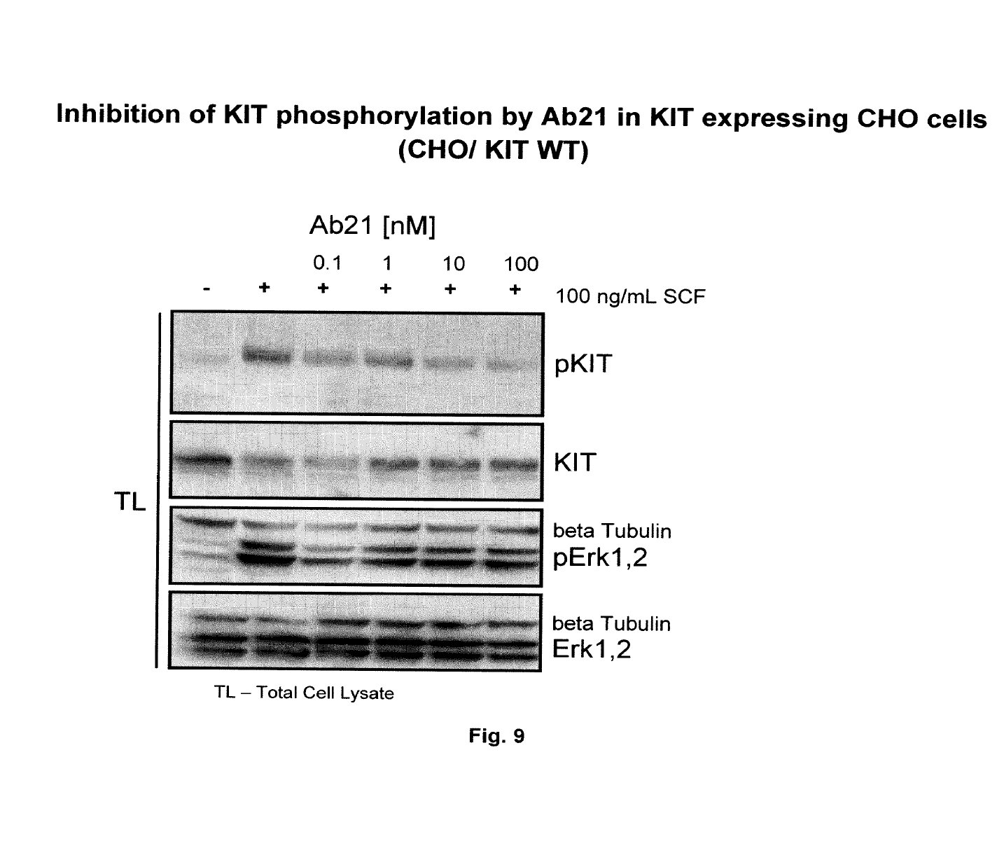

FIG. 9 shows that KIT phosphorylation in CHO/KIT WT cells in response to SCF was inhibited by antibody Ab21 in a dose-dependent manner.

### 6.10 Example 10

### Inhibition of Colony Formation by KIT Expressing Cho Cells in Soft Agar Assay

Full-length IgG1 human antibody Ab21 was also tested for its ability to inhibit anchorage independent cell growth in soft agar assays of CHO/KIT WT cells. Soft agar assay for colony formation is an anchorage independent growth assay, which is a useful assay for detecting malignant transformation of cells. In vitro transformation is associated with certain phenotypic changes such as loss of contact inhibition (cells can grow over one another) and anchorage independence (cells form colonies in soft agar). In general, nontransformed cells fail to grow when suspended in a viscous fluid or gel (e.g. agar or agarose), however when these cell are transformed, they are able to grow in a viscous fluid or gel and become anchorage-independent. The process by which these phenotypic changes occur, is assumed to be closely related to the process of in vivo carcinogenesis.

The soft agar assays were carried out as follows. Base agar layer (containing agar and cell culture medium) was added to each well of a 96 well plate. Cell agar layer (containing agar, cell culture medium and cell suspension) was added on top of the base agar layer. Anti-KIT antibody Ab21 was diluted in cell culture medium supplemented and not supplemented with 30 ng/mL of the KIT ligand SCF. In parallel as a control, medium without antibody supplemented and not supplemented with 30 ng/mL of the KIT ligand SCF was pipetted onto the base agar layer. Plates were incubated at 37° C. and 5% CO₂ for 5-8 days.

When treatment was completed, the agars were solubilized and the cells were lysed. The green fluorescent Cyquant® GR dye was mixed with the lysates. This dye exhibits fluorescence when bound to cellular nucleic acids. Fluorescence was measured at 480 nm excitation and 520 nm emission.

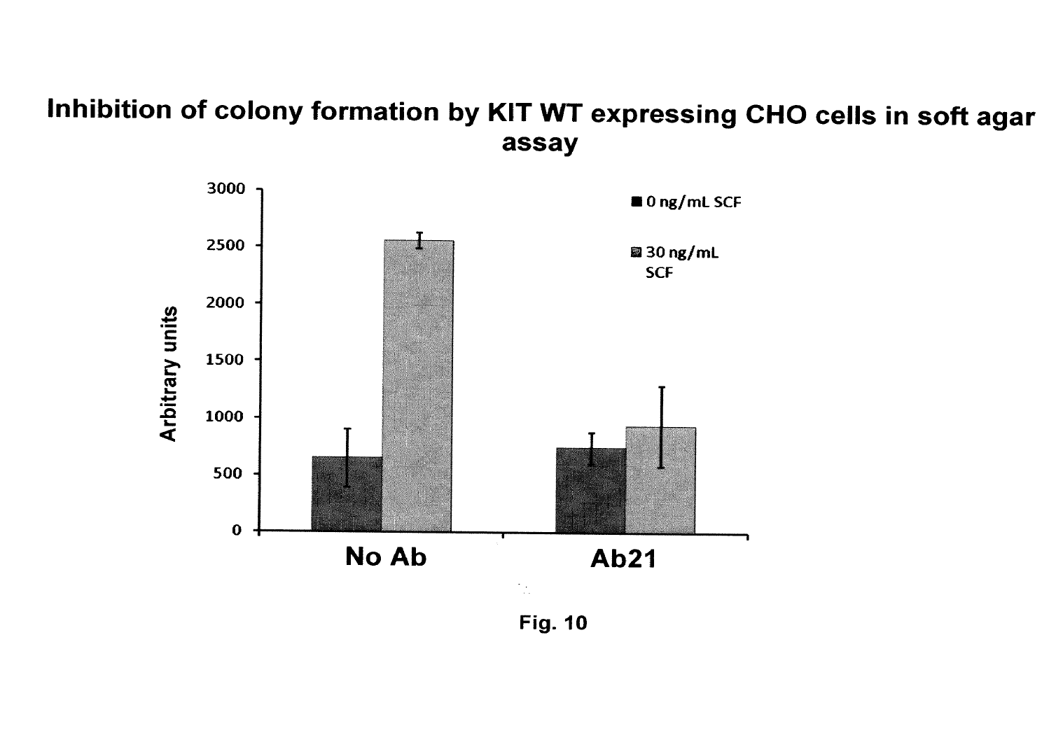

The results from the soft agar assays are depicted in FIG. 10. The results in FIG. 10 shows that antibody Ab21 was able to inhibit soft agar formation.

### 6.11 Example 11

### Antibody Ab1 is Internalized in CHO Cells and NIH3T3 Cells Expressing Exogenous Human, Wild-Type KIT

Immunofluorescence staining assays were carried to determine if antibody Ab1 was internalized by cells. The immunofluorescence staining assays were carried out essentially as described below. Materials and reagents for the immunofluorescence assays include the following:
Tissue culture slides: Poly-D-Lysine coated four or eight well chamber slide
Antibody: Antibody Ab1 conjugated with the fluorescent dye Alexa-488 (Invitrogen)
Fixative: 4% paraformaldehyde; store 40% PFA microscopy grade in fridge and dilute 1:10 with PBS just before use
Mounting media: Prolong Gold antifade+DAPI
CHO cells engineered to express exogenous human, wild-type KIT (full-length) (“CHO/KIT”)
NIH3T3 cells engineered to express exogenous human, wild-type KIT (full-length)
(“NIH3T3/KIT”)

15,000 to 30,000 CHO/KIT cells or NIH3T3/KIT cells were seeded into each well of the tissue culture slide. The cells were cultivated for at least 24 hrs before starvation in media containing no fetal bovin serum overnight. Ab1-Alexa488 was diluted in starvation media containing 1% bovine serum albumin and pipetted onto the cell layer. 5 to 60 min later, cell layers were washed once with PBS (room temperature). Cell layers were fixed for 18 min with fixative at room temperature, and were washed 3 times with PBS. The cells were mounted between slide and cover slip and kept at room temperature over night. Internalization of the antibody was analyzed by confocal microscopy.

The immunofluorescence staining assays showed that antibody Ab1 bound to the surface of CHO/KIT cells and NIH3T3/KIT cells, and was internalized CHO/KIT cells and NIH3T3/KIT cells.

The embodiments described herein are intended to be merely exemplary, and those skilled in the art will recognize, or be able to ascertain using no more than routine experimentation, numerous equivalents to the specific procedures described herein. All such equivalents are considered to be within the scope of the present invention and are covered by the following claims. Furthermore, as used in this specification and claims, the singular forms “a,” “an” and “the” include plural forms unless the content clearly dictates otherwise. Thus, for example, reference to “an antibody” includes a mixture of two or more such antibodies, and the like. Additionally, ordinarily skilled artisans will recognize that operational sequences must be set forth in some specific order for the purpose of explanation and claiming, but the present invention contemplates various changes beyond such specific order.

All references cited herein are incorporated herein by reference in their entirety and for all purposes to the same extent as if each individual publication or patent or patent application was specifically and individually indicated to be incorporated by reference in its entirety for all purposes.

1. An isolated antibody, which immunospecifically binds to a D4/D5 region of human KIT (SEQ ID NO: 171), comprising:
(A) (i) a variable light (“VL”) chain region comprising a VL CDR1, VL CDR2, and VL CDR3 having the amino acid sequences of SEQ ID NO: 2 (VL CDR1 of Ab1), SEQ ID NO: 14 (VL CDR2 of Ab1), and SEQ ID NO: 26 (VL CDR3 of Ab1), respectively; and (ii) a variable heavy (“VH”) chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively; wherein said antibody comprises 0, 1, or 2 conservative amino acid substitutions in any one of VL CDR1, VL CDR2, VL CDR3, VH CDR1, VH CDR2, and VH CDR3;
(B) (i) a VL chain region comprising a VL CDR1, VL CDR2, and VL CDR3 having the amino acid sequences of SEQ ID NO: 2 (VL CDR1 of Ab1), SEQ ID NO: 14 (VL CDR2 of Ab1), and SEQ ID NO: 26 (VL CDR3 of Ab1), respectively, wherein said VL chain region comprises 0, 1, or 2 conservative amino acid substitutions in any one of VL CDR1, VL CDR2, or VL CDR3; and (ii) a VH chain region comprising an amino acid sequence that has at least 80% amino acid seqeuence identity to SEQ ID NO: 188;
(C) (i) a VL chain region comprising a VL CDR1, VL CDR2, and VL CDR3 having the amino acid sequences of SEQ ID NO: 330 (VL CDR1 of Ab21), SEQ ID NO: 331 (VL CDR2 of Ab21), and SEQ ID NO: 332 (VL CDR3 of Ab21), respectively; and (ii) a VH chain region comprising a VH CDR1, VH CDR2, and VH CDR3 having the amino acid sequences of SEQ ID NO: 337 (VH CDR1 of Ab21), SEQ ID NO: 338 (VH CDR2 of Ab21), and SEQ ID NO: 339 (VH CDR3 of Ab21), respectively, wherein said antibody comprises 0, 1, or 2 conservative amino acid substitutions in any one of VL CDR1, VL CDR2, VL CDR3, VH CDR1, VH CDR3, and VH CDR3;
(D) (i) a VL chain region comprising a VL CDR1, VL CDR2, and VL CDR3 having the amino acid sequences of SEQ ID NO: 2 (VL CDR1 of Ab1), SEQ ID NO: 14 (VL CDR2 of Ab1), and SEQ ID NO: 26 (VL CDR3 of Ab1), respectively; and (ii) a VH chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively;
(E) (i) a VL chain region comprising a VL CDR1, VL CDR2, and VL CDR3 having the amino acid sequences of SEQ ID NO: 330 (VL CDR1 of Ab21), SEQ ID NO: 331 (VL CDR2 of Ab21), and SEQ ID NO: 332 (VL CDR3 of Ab21), respectively; and (ii) a VH chain region comprising a VH CDR1, VH CDR2, and VH CDR3 having the amino acid sequences of SEQ ID NO: 337 (VH CDR1 of Ab21), SEQ ID NO: 338 (VH CDR2 of Ab21), and SEQ ID NO: 339 (VH CDR3 of Ab21), respectively;
(F) (i) a VL chain region comprising a VL CDR1, VL CDR2, and VL CDR3 having the amino acid sequences of SEQ ID NO: 3 (VL CDR1 of Ab2), SEQ ID NO: 15 (VL CDR2 of Ab2), and SEQ ID NO: 27 (VL CDR3 of Ab2), respectively; and (ii) a VH chain region comprising a VH CDR1, VH CDR2, and VH CDR3 having the amino acid sequences of SEQ ID NO: 87 (VH CDR1 of Ab2), SEQ ID NO: 99 (VH CDR2 of Ab2), and SEQ ID NO: 111 (VH CDR3 of Ab2), respectively;
(G) (i) a VL chain region comprising a VL CDR1, VL CDR2, and VL CDR3 having the amino acid sequences of SEQ ID NO: 4 (VL CDR1 of Ab3), SEQ ID NO: 16 (VL CDR2 of Ab3), and SEQ ID NO: 28 (VL CDR3 of Ab3), respectively; and (ii) a VH chain region comprising a VH CDR1, VH CDR2, and VH CDR3 having the amino acid sequences of SEQ ID NO: 88 (VH CDR1 of Ab3), SEQ ID NO: 100 (VH CDR2 of Ab3), and SEQ ID NO: 112 (VH CDR3 of Ab3), respectively;
(H) (i) a VL chain region comprising a VL CDR1, VL CDR2, and VL CDR3 having the amino acid sequences of SEQ ID NO: 5 (VL CDR1 of Ab4), SEQ ID NO: 17 (VL CDR2 of Ab4), and SEQ ID NO: 29 (VL CDR3 of Ab4), respectively; and (ii) a VH chain region comprising a VH CDR1, VH CDR2, and VH CDR3 having the amino acid sequences of SEQ ID NO: 89 (VH CDR1 of Ab4), SEQ ID NO: 101 (VH CDR2 of Ab11), and SEQ ID NO: 113 (VH CDR3 of Ab4), respectively;
(I) (i) a VL chain region comprising a VL CDR1, VL CDR2, and VL CDR3 having the amino acid sequences of SEQ ID NO: 6 (VL CDR1 of Ab5), SEQ ID NO: 18 (VL CDR2 of Ab5), and SEQ ID NO: 30 (VL CDR3 of Ab5), respectively; and (ii) a VH chain region comprising a VH CDR1, VH CDR2, and VH CDR3 having the amino acid sequences of SEQ ID NO: 90 (VH CDR1 of Ab5), SEQ ID NO: 102 (VH CDR2 of Ab5), and SEQ ID NO: 114 (VH CDR3 of Ab5), respectively.
(J) (i) a VL chain region comprising a VL CDR1, VL CDR2, and VL CDR3 having the amino acid sequences of SEQ ID NO: 7 (VL CDR1 of Ab6), SEQ ID NO: 19 (VL CDR2 of Ab6), and SEQ ID NO: 31 (VL CDR3 of Ab6), respectively; and (ii) a VH chain region comprising a VH CDR1, VH CDR2, and VH CDR3 having the amino acid sequences of SEQ ID NO: 91 (VH CDR1 of Ab6), SEQ ID NO: 103 (VH CDR2 of Ab6), and SEQ ID NO: 115 (VH CDR3 of Ab6), respectively;
(K) (i) a VL chain region comprising a VL CDR1, VL CDR2, and VL CDR3 having the amino acid sequences of SEQ ID NO: 8 (VL CDR1 of Ab7), SEQ ID NO: 20 (VL CDR2 of Ab7), and SEQ ID NO: 32 (VL CDR3 of Ab7), respectively; and (ii) a VH chain region comprising a VH CDR1, VH CDR2, and VH CDR3 having the amino acid sequences of SEQ ID NO: 92 (VH CDR1 of Ab7), SEQ ID NO: 104 (VH CDR2 of Ab7), and SEQ ID NO: 116 (VH CDR3 of Ab7), respectively;
(L) (i) a VL chain region comprising a VL CDR1, VL CDR2, and VL CDR3 having the amino acid sequences of SEQ ID NO: 9 (VL CDR1 of Ab8), SEQ ID NO: 21 (VL CDR2 of Ab8), and SEQ ID NO: 33 (VL CDR3 of Ab8), respectively; and (ii) a VH chain region comprising a VH CDR1, VH CDR2, and VH CDR3 having the amino acid sequences of SEQ ID NO: 93 (VH CDR1 of Ab8), SEQ ID NO: 105 (VH CDR2 of Ab8), and SEQ ID NO: 117 (VH CDR3 of Ab8), respectively;
(M) (i) a VL chain region comprising a VL CDR1, VL CDR2, and VL CDR3 having the amino acid sequences of SEQ ID NO: 10 (VL CDR1 of Ab9), SEQ ID NO: 22 (VL CDR2 of Ab9), and SEQ ID NO: 34 (VL CDR3 of Ab9), respectively; and (ii) a VH chain region comprising a VH CDR1, VH CDR2, and VH CDR3 having the amino acid sequences of SEQ ID NO: 94 (VH CDR1 of Ab9), SEQ ID NO: 106 (VH CDR2 of Ab9), and SEQ ID NO: 118 (VH CDR3 of Ab9), respectively;
(N) (i) a VL chain region comprising a VL CDR1, VL CDR2, and VL CDR3 having the amino acid sequences of SEQ ID NO: 11 (VL CDR1 of Ab10), SEQ ID NO: 23 (VL CDR2 of Ab10), and SEQ ID NO: 35 (VL CDR3 of Ab10), respectively; and (ii) a VH chain region comprising a VH CDR1, VH CDR2, and VH CDR3 having the amino acid sequences of SEQ ID NO: 95 (VH CDR1 of Ab10), SEQ ID NO: 107 (VH CDR2 of Ab10), and SEQ ID NO: 119 (VH CDR3 of Ab10), respectively;
(O) (i) a VL chain region comprising a VL CDR1, VL CDR2, and VL CDR3 having the amino acid sequences of SEQ ID NO: 12 (VL CDR1 of Ab11), SEQ ID NO: 24 (VL CDR2 of Ab11), and SEQ ID NO: 36 (VL CDR2 of Ab11), respectively; and (ii) a VH chain region comprising a VH CDR1, VH CDR2, and VH CDR3 having the amino acid sequences of SEQ ID NO: 96 (VH CDR1 of Ab11), SEQ ID NO: 108 (VH CDR2 of Ab11), and SEQ ID NO: 120 (VH CDR3 of Ab11), respectively; or
(P) (i) a VL chain region comprising a VL CDR1, VL CDR2, and VL CDR3 having the amino acid sequences of SEQ ID NO: 13 (VL CDR1 of Ab12), SEQ ID NO: 25 (VL CDR2 of Ab12), and SEQ ID NO: 37 (VL CDR3 of Ab12), respectively; and (ii) a VH chain region comprising a VH CDR1, VH CDR2, and VH CDR3 having the amino acid sequences of SEQ ID NO: 97 (VH CDR1 of Ab12), SEQ ID NO: 109 (VH CDR2 of Ab12), and SEQ ID NO: 121 (VH CDR3 of Ab12), respectively.

2. The isolated antibody of claim 1, comprising:
(A) a variable light (“VL”) chain region comprising the amino acid sequence of SEQ ID NO: 176 (VL domain of Ab1), and a variable heavy (“VH”) chain region comprising the amino acid sequence of SEQ ID NO: 188 (VH domain of Ab1);
(B) a VL chain region comprising the amino acid sequence of SEQ ID NO: 176 (VL domain of Ab1) starting at the second amino acid residue of SEQ ID NO: 176, and a VH chain region comprising the amino acid sequence of SEQ ID NO: 188 (VH domain of Ab1);
(C) a VL chain region comprising the amino acid sequence of SEQ ID NO: 963 (VL domain of Ab1), and a VH chain region comprising the amino acid sequence of SEQ ID NO: 188 (VH domain of Ab1);
(D) a VL chain region comprising the amino acid sequence of SEQ ID NO: 328 (VL domain of Ab21), and a VH chain region comprising the amino acid sequence of SEQ ID NO: 329 (VH domain of Ab21);
(E) a VL chain region comprising the amino acid sequence of SEQ ID NO: 964 (VL domain of Ab21), and a VH chain region comprising the amino acid sequence of SEQ ID NO: 329 (VH domain of Ab21);
(F) a VL chain region comprising the amino acid sequence of SEQ ID NO: 177 (VL domain of Ab2) or the amino acid sequence of SEQ ID NO: 177 starting at the second amino acid residue, and a VH chain region comprising the amino acid sequence of SEQ ID NO: 189 (VH domain of Ab2);
(G) a VL chain region comprising the amino acid sequence of SEQ ID NO: 178 (VL domain of Ab3) or the amino acid sequence of SEQ ID NO: 178 starting at the second amino acid residue, and a VH chain region comprising the amino acid sequence of SEQ ID NO: 190 (VH domain of Ab3);
(H) a VL chain region comprising the amino acid sequence of SEQ ID NO: 179 (VL domain of Ab4) or the amino acid sequence of SEQ ID NO: 179 starting at the second amino acid residue, and a VH chain region comprising the amino acid sequence of SEQ ID NO: 191 (VH domain of Ab4);
(I) a VL chain region comprising the amino acid sequence of SEQ ID NO: 180 (VL domain of Ab5) or the amino acid sequence of SEQ ID NO: 180 starting at the second amino acid residue, a VH chain region comprising the amino acid sequence of SEQ ID NO: 192 (VH domain of Ab5);
(J) a VL chain region comprising the amino acid sequence of SEQ ID NO: 181 (VL domain of Ab6) or the amino acid sequence of SEQ ID NO: 181 starting at the second amino acid residue, and a VH chain region comprising the amino acid sequence of SEQ ID NO: 193 (VH domain of Ab6);
(K) a VL chain region comprising the amino acid sequence of SEQ ID NO: 182 (VL domain of Ab7) or the amino acid sequence of SEQ ID NO: 182 starting at the second amino acid residue, and a VH chain region comprising the amino acid sequence of SEQ ID NO: 194 (VH domain of Ab7);
(L) a VL chain region comprising the amino acid sequence of SEQ ID NO: 183 (VL domain of Ab8) or the amino acid sequence of SEQ ID NO: 183 starting at the second amino acid residue, and a VH chain region comprising the amino acid sequence of SEQ ID NO: 195 (VH domain of Ab8);
(M) a VL chain region comprising the amino acid sequence of SEQ ID NO: 184 (VL domain of Ab9) or the amino acid sequence of SEQ ID NO: 184 starting at the second amino acid residue, and comprises a VH chain region comprising the amino acid sequence of SEQ ID NO: 196 (VH domain of Ab9);
(N) a VL chain region comprising the amino acid sequence of SEQ ID NO: 185 (VL domain of Ab10) or the amino acid sequence of SEQ ID NO: 185 starting at the second amino acid residue, and a VH chain region comprising the amino acid sequence of SEQ ID NO: 197 (VH domain of Ab10);
(O) a VL chain region comprising the amino acid sequence of SEQ ID NO: 186 (VL domain of Ab11) or the amino acid sequence of SEQ ID NO: 186 starting at the second amino acid residue, and a VH chain region comprising the amino acid sequence of SEQ ID NO: 198 (VH domain of Ab11); or
(P) a VL chain region comprising the amino acid sequence of SEQ ID NO: 187 (VL domain of Ab12) or the amino acid sequence of SEQ ID NO: 187 starting at the second amino acid residue, and a VH chain region comprising the amino acid sequence of SEQ ID NO: 199 (VH domain of Ab12).

3. The isolated antibody of claim 1, comprising:
(A) a VL chain region comprising a VL CDR1, VL CDR2, and VL CDR3 having the amino acid sequences of SEQ ID NO: 2 (VL CDR1 of Ab1), SEQ ID NO: 14 (VL CDR2 of Ab1), and SEQ ID NO: 26 (VL CDR3 of Ab1), respectively; and a VH chain region comprising the amino acid sequence of SEQ ID NO: 188 (Ab1);
(B) a VL chain region comprising a VL CDR1, VL CDR2, and VL CDR3 having the amino acid sequences of SEQ ID NO: 330 (VL CDR1 of Ab21), SEQ ID NO: 331 (VL CDR2 of Ab21), and SEQ ID NO: 332 (VL CDR3 of Ab21), respectively; and a VH chain region comprising the amino acid sequence of SEQ ID NO: 329 (Ab21);
(C) a VL chain region comprising a VL CDR1, VL CDR2, and VL CDR3 having the amino acid sequences of SEQ ID NO: 3 (VL CDR1 of Ab2), SEQ ID NO: 15 (VL CDR2 of Ab2), and SEQ ID NO: 27 (VL CDR3 of Ab2), respectively; and a VH chain region comprising the amino acid sequence of SEQ ID NO: 189 (Ab2);
(D) a VL chain region comprising a VL CDR1, VL CDR2, and VL CDR3 having the amino acid sequences of SEQ ID NO: 4 (VL CDR1 of Ab3), SEQ ID NO: 16 (VL CDR2 of Ab6), and SEQ ID NO: 28 (VL CDR3 of Ab3), respectively; and a VH chain region comprising the amino acid sequence of SEQ ID NO: 190 (Ab3);
(E) a VL chain region comprising a VL CDR1, VL CDR2, and VL CDR3 having the amino acid sequences of SEQ ID NO: 5 (VL CDR1 of Ab4), SEQ ID NO: 17 (VL CDR2 of Ab4), and SEQ ID NO: 29 (VL CDR3 of Ab4), respectively; and a VH chain region comprising the amino acid sequence of SEQ ID NO: 191 (Ab4);
(F) a VL chain region comprising a VL CDR1, VL CDR2, and VL CDR3 having the amino acid sequences of SEQ ID NO: 6 (VL CDR1 of Ab5), SEQ ID NO: 18 (VL CDR2 of Ab5), and SEQ ID NO: 30 (VL CDR3 of Ab5), respectively; and a VH chain region comprising the amino acid sequence of SEQ ID NO: 192 (Ab5);
(G) a VL chain region comprising a VL CDR1, VL CDR2, and VL CDR3 having the amino acid sequences of SEQ ID NO: 7 (VL CDR1 of Ab6), SEQ ID NO: 19 (VL CDR2 of Ab6), and SEQ ID NO: 31 (VL CDR3 of Ab6), respectively; and a VH chain region comprising the amino acid sequence of SEQ ID NO: 193 (Ab6);
(H) a VL chain region comprising a VL CDR1, VL CDR2, and VL CDR3 having the amino acid sequences of SEQ ID NO: 8 (VL CDR1 of Ab7), SEQ ID NO: 20 (VL CDR2 of Ab7), and SEQ ID NO: 32 (VL CDR3 of Ab7), respectively; and a VH chain region comprising the amino acid sequence of SEQ ID NO: 194 (Ab7);
(I) a VL chain region comprising a VL CDR1, VL CDR2, and VL CDR3 having the amino acid sequences of SEQ ID NO: 9 (VL CDR1 of Ab8), SEQ ID NO: 21 (VL CDR2 of Ab8), and SEQ ID NO: 33 (VL CDR3 of Ab8), respectively; and a VH chain region comprising the amino acid sequence of SEQ ID NO: 195 (Ab8);
(J) a VL chain region comprising a VL CDR1, VL CDR2, and VL CDR3 having the amino acid sequences of SEQ ID NO: 10 (VL CDR1 of Ab9), SEQ ID NO: 22 (VL CDR2 of Ab9), and SEQ ID NO: 34 (VL CDR3 of Ab9), respectively; and a VH chain region comprising the amino acid sequence of SEQ ID NO: 196 (Ab9);
(K) a VL chain region comprising a VL CDR1, VL CDR2, and VL CDR3 having the amino acid sequences of SEQ ID NO: 11 (VL CDR1 of Ab10), SEQ ID NO: 23 (VL CDR2 of Ab10), and SEQ ID NO: 35 (VL CDR3 of Ab10), respectively; and a VH chain region comprising the amino acid sequence of SEQ ID NO: 197 (Ab10);
(L) a VL chain region comprising a VL CDR1, VL CDR2, and VL CDR3 having the amino acid sequences of SEQ ID NO: 12 (VL CDR1 of Ab11), SEQ ID NO: 24 (VL CDR2 of Ab11), and SEQ ID NO: 36 (VL CDR3 of Ab11), respectively; and a VH chain region comprising the amino acid sequence of SEQ ID NO: 198 (Ab11); or
(M) a VL chain region comprising a VL CDR1, VL CDR2, and VL CDR3 having the amino acid sequences of SEQ ID NO: 13 (VL CDR1 of Ab12), SEQ ID NO: 25 (VL CDR2 of Ab12), and SEQ ID NO: 37 (VL CDR3 of Ab12), respectively; and a VH chain region comprising the amino acid sequence of SEQ ID NO: 199 (Ab12).

4. The isolated antibody of claim 1, comprising:
(A) a VL chain region comprising the amino acid sequence of SEQ ID NO: 176 (Ab1); and a VH chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively;
(B) a VL chain region comprising the amino acid sequence of SEQ ID NO: 176 (Ab1) starting at the second amino acid residue of SEQ ID NO: 176; and a VH chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively;
(C) a VL chain region comprising the amino acid sequence of SEQ ID NO: 328 (Ab21); and a VH chain region comprising a VH CDR1, VH CDR2, and VH CDR3 having the amino acid sequences of SEQ ID NO: 337 (VH CDR1 of Ab21), SEQ ID NO: 338 (VH CDR2 of Ab21), and SEQ ID NO: 339 (VH CDR3 of Ab12), respectively;
(D) a VL chain region comprising the amino acid sequence of SEQ ID NO: 177 (Ab2) or the amino acid sequence of SEQ ID NO: 177 starting at the second amino acid residue; and a VH chain region comprising a VH CDR1, VH CDR2, and VH CDR3 having the amino acid sequences of SEQ ID NO: 87 (VH CDR1 of Ab2), SEQ ID NO: 99 (VH CDR2 of Ab2), and SEQ ID NO: 111 (VH CDR3 of Ab2), respectively;
(E) a VL chain region comprising the amino acid sequence of SEQ ID NO: 178 (Ab3) or the amino acid sequence of SEQ ID NO: 178 starting at the second amino acid residue; and a VH chain region comprising a VH CDR1, VH CDR2, and VH CDR3 having the amino acid sequences of SEQ ID NO: 88 (VH CDR1 of Ab3), SEQ ID NO: 100 (VH CDR2 of Ab3), and SEQ ID NO: 112 (VH CDR3 of Ab3), respectively;
(F) a VL chain region comprising the amino acid sequence of SEQ ID NO: 179 (Ab4) or the amino acid sequence of SEQ ID NO: 179 starting at the second amino acid residue; and a VH chain region comprising a VH CDR1, VH CDR2, and VH CDR3 having the amino acid sequences of SEQ ID NO: 89 (VH CDR1 of Ab4), SEQ ID NO: 101 (VH CDR2 of Ab4), and SEQ ID NO: 113 (VH CDR3 of Ab4), respectively;
(G) a VL chain region comprising the amino acid sequence of SEQ ID NO: 180 (Ab5) or the amino acid sequence of SEQ ID NO: 180 starting at the second amino acid residue; and a VH chain region comprising a VH CDR1, VH CDR2, and VH CDR3 having the amino acid sequences of SEQ ID NO: 90 (VH CDR1 of Ab5), SEQ ID NO: 102 (VH CDR2 of Ab5), and SEQ ID NO: 114 (VH CDR3 of Ab5), respectively;
(H) a VL chain region comprising the amino acid sequence of SEQ ID NO: 181 (Ab6) or the amino acid sequence of SEQ ID NO: 181 starting at the second amino acid residue; and a VH chain region comprising a VH CDR1, VH CDR2, and VH CDR3 having the amino acid sequences of SEQ ID NO: 91 (VH CDR1 of Ab6), SEQ ID NO: 103 (VH CDR2 of Ab6), and SEQ ID NO: 115 (VH CDR3 of Ab6), respectively;
(I) a VL chain region comprising the amino acid sequence of SEQ ID NO: 182 (Ab7) or the amino acid sequence of SEQ ID NO: 182 starting at the second amino acid residue; and a VH chain region comprising a VH CDR1, VH CDR2, and VH CDR3 having the amino acid sequences of SEQ ID NO: 92 (VH CDR1 of Ab7), SEQ ID NO: 104 (VH CDR2 of Ab7), and SEQ ID NO: 116 (VH CDR3 of Ab7), respectively;
(J) a VL chain region comprising the amino acid sequence of SEQ ID NO: 183 (Ab8) or the amino acid sequence of SEQ ID NO: 183 starting at the second amino acid residue; and a VH chain region comprising a VH CDR1, VH CDR2, and VH CDR3 having the amino acid sequences of SEQ ID NO: 93 (VH CDR1 of Ab8), SEQ ID NO: 105 (VH CDR2 of Ab8), and SEQ ID NO: 117 (VH CDR3 of Ab8), respectively;
(K) a VL chain region comprising the amino acid sequence of SEQ ID NO: 184 (Ab9) or the amino acid sequence of SEQ ID NO: 184 starting at the second amino acid residue; and a VH chain region comprising a VH CDR1, VH CDR2, and VH CDR3 having the amino acid sequences of SEQ ID NO: 94 (VH CDR1 of Ab9), SEQ ID NO: 106 (VH CDR2 of Ab9), and SEQ ID NO: 118 (VH CDR3 of Ab9), respectively;
(L) a VL chain region comprising the amino acid sequence of SEQ ID NO: 185 (Ab10) or the amino acid sequence of SEQ ID NO: 185 starting at the second amino acid residue; and a VH chain region comprising a VH CDR1, VH CDR2, and VH CDR3 having the amino acid sequences of SEQ ID NO: 95 (VH CDR1 of Ab10), SEQ ID NO: 107 (VH CDR2 of Ab10), and SEQ ID NO: 119 (VH CDR3 of Ab10), respectively;
(M) a VL chain region comprising the amino acid sequence of SEQ ID NO: 186 (Ab11) or the amino acid sequence of SEQ ID NO: 186 starting at the second amino acid residue; and a VH chain region comprising a VH CDR1, VH CDR2, and VH CDR3 having the amino acid sequences of SEQ ID NO: 96 (VH CDR1 of Ab11), SEQ ID NO: 108 (VH CDR2 of Ab11), and SEQ ID NO: 120 (VH CDR3 of Ab11), respectively; or
(N) a VL chain region comprising the amino acid sequence of SEQ ID NO: 187 (Ab12) or the amino acid sequence of SEQ ID NO: 187 starting at the second amino acid residue; and a VH chain region comprising a VH CDR1, VH CDR2, and VH CDR3 having the amino acid sequences of SEQ ID NO: 97 (VH CDR1 of Ab12), SEQ ID NO: 109 (VH CDR2 of Ab12), and SEQ ID NO: 121 (VH CDR3 of Ab12), respectively.

5. An isolated antibody, which immunospecifically binds to a D4/D5 region of human KIT (SEQ ID NO: 171), wherein said isolated antibody competes for binding to the D4/D5 region of human KIT with an antibody comprising:
(A) a VL chain region comprising the amino acid sequence of SEQ ID NO: 176 (VL domain of Ab1), and a VH chain region comprising the amino acid sequence of SEQ ID NO: 188 (VH domain of Ab1);
(B) a VL chain region comprising the amino acid sequence of SEQ ID NO: 176 (VL domain of Ab1) starting at the second amino acid residue of SEQ ID NO: 176, and a VH chain region comprising the amino acid sequence of SEQ ID NO: 188 (VH domain of Ab1);
(C) a VL chain region comprising the amino acid sequence of SEQ ID NO: 963 (VL domain of Ab1), and a VH chain region comprising the amino acid sequence of SEQ ID NO: 188 (VH domain of Ab1);
(D) a VL chain region comprising the amino acid sequence of SEQ ID NO: 328 (VL domain of Ab21), and a VH chain region comprising the amino acid sequence of SEQ ID NO: 329 (VH domain of Ab21);
(E) a VL chain region comprising the amino acid sequence of SEQ ID NO: 964 (VL domain of Ab21), and a VH chain region comprising the amino acid sequence of SEQ ID NO: 329 (VH domain of Ab21);
(F) a VL chain region comprising the amino acid sequence of SEQ ID NO: 177 (VL domain of Ab2) and a VH chain region comprising the amino acid sequence of SEQ ID NO: 189 (VH domain of Ab2);
(G) a VL chain region comprising the amino acid sequence of SEQ ID NO: 178 (VL domain of Ab3) and a VH chain region comprising the amino acid sequence of SEQ ID NO: 190 (VH domain of Ab3);
(H) a VL chain region comprising the amino acid sequence of SEQ ID NO: 179 (VL domain of Ab4) and a VH chain region comprising the amino acid sequence of SEQ ID NO: 191 (VH domain of Ab4);
(I) a VL chain region comprising the amino acid sequence of SEQ ID NO: 180 (VL domain of Ab5) and a VH chain region comprising the amino acid sequence of SEQ ID NO: 192 (VH domain of Ab5);
(J) a VL chain region comprising the amino acid sequence of SEQ ID NO: 181 (VL domain of Ab6) and a VH chain region comprising the amino acid sequence of SEQ ID NO: 193 (VH domain of Ab6);
(K) a VL chain region comprising the amino acid sequence of SEQ ID NO: 182 (VL domain of Ab7) and a VH chain region comprising the amino acid sequence of SEQ ID NO: 194 (VH domain of Ab7);
(L) a VL chain region comprising the amino acid sequence of SEQ ID NO: 183 (VL domain of Ab8) and a VH chain region comprising the amino acid sequence of SEQ ID NO: 195 (VH domain of Ab8);
(M) a VL chain region comprising the amino acid sequence of SEQ ID NO: 184 (VL domain of Ab9) and comprises a VH chain region comprising the amino acid sequence of SEQ ID NO: 196 (VH domain of Ab9);
(N) a VL chain region comprising the amino acid sequence of SEQ ID NO: 185 (VL domain of Ab10) and a VH chain region comprising the amino acid sequence of SEQ ID NO: 197 (VH domain of Ab10);
(O) a VL chain region comprising the amino acid sequence of SEQ ID NO: 186 (VL domain of Ab11) and a VH chain region comprising the amino acid sequence of SEQ ID NO: 198 (VH domain of Ab11); or
(P) a VL chain region comprising the amino acid sequence of SEQ ID NO: 187 (VL domain of Ab12) and a VH chain region comprising the amino acid sequence of SEQ ID NO: 199 (VH domain of Ab12).

6. The isolated antibody of claim 5, wherein said isolated antibody competes with an antibody comprising (i) a VL chain region comprising the amino acid sequence of SEQ ID NO: 328 (VL domain of Ab21), and a VH chain region comprising the amino acid sequence of SEQ ID NO: 329 (VH domain of Ab21); or (ii) a VL chain region comprising the amino acid sequence of SEQ ID NO: 176 (VL domain of Ab1) starting at the second amino acid residue of SEQ ID NO: 176, and a VH chain region comprising the amino acid sequence of SEQ ID NO: 188 (VH domain of Ab1).

7. (canceled)

8. (canceled)

9. (canceled)

10. (canceled)

11. (canceled)

12. (canceled)

13. (canceled)

14. (canceled)

15. (canceled)

16. The isolated antibody of claim 1, further comprising a human light chain constant region, wherein the human light chain constant region is a human kappa light chain constant region, or comprises the amino acid sequence of SEQ ID NO: 344 or 965.

17. The isolated antibody of claim 1, further comprising a human heavy chain constant region, wherein the human heavy chain constant region is a human gamma heavy chain constant region, or comprises the amino acid sequence of SEQ ID NO: 345.

18. (canceled)

19. The isolated antibody of claim 1, wherein the antibody is a Fab antibody or an antigen binding fragment.

20. The isolated antibody of claim 1, wherein the antibody is a human IgG1 or IgG4 antibody.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. A conjugate comprising the antibody of claim 1, or a KIT-binding fragment thereof, linked to a therapeutic agent.

33. (canceled)

34. (canceled)

35. (canceled)

36. A pharmaceutical composition comprising the conjugate of claim 32 and a pharmaceutically acceptable carrier.

37. An isolated polynucleotide comprising nucleotide sequences encoding a VH chain region, a VL chain region, or both a VL chain region and a VH chain region, of an isolated antibody of claim 1.

38. The isolated polynucleotide of claim 37, wherein the isolated polynucleotide comprises a nucleotide sequence encoding a VH chain region, a VL chain region, or both a VL chain region and a VH chain region, of an antibody comprising:
(i) a VL chain region comprising a VL CDR1, VL CDR2, and VL CDR3 having the amino acid sequences of SEQ ID NO: 2 (VL CDR1 of Ab1), SEQ ID NO: 14 (VL CDR2 of Ab1), and SEQ ID NO: 26 (VL CDR3 of Ab1) or SEQ ID NO: 332, respectively; and (ii) a VH chain region comprising a VH CDR1, VH CDR2, and VH CDR3 having the amino acid sequences of SEQ ID NO: 86 (VH CDR1 of Ab1), SEQ ID NO: 98 (VH CDR2 of Ab1), and SEQ ID NO: 110 (VH CDR3 of Ab1), respectively.

39. (canceled)

40. (canceled)

41. (canceled)

42. An expression vector comprising the isolated polynucleotide of claim 37.

43. (canceled)

44. A host cell comprising the expression vector of claim 42.

45. A pharmaceutical composition comprising the isolated antibody of claim 1 and a pharmaceutically acceptable carrier.

46. A kit comprising the isolated antibody of claim 1.

47. A kit comprising the conjugate of claim 32.

48. A method for treating or managing a KIT-mediated disorder, comprising administering to a subject in need thereof a therapeutically effective amount of the isolated antibody of claim 1.

49. (canceled)

50. (canceled)

51. A method for treating or managing a KIT-mediated disorder, comprising administering to a subject in need thereof a therapeutically effective amount of the conjugate of claim 32.

52. The method of claim 48, wherein the KIT-mediated disorder is cancer, an inflammatory condition, or fibrosis.

53. The method of claim 52, wherein the KIT-mediated disorder is cancer, and the cancer is leukemia, chronic myelogenous leukemia, lung cancer, small cell lung cancer, or gastrointestinal stromal tumors.

54. (canceled)

55. (canceled)

56. The method of claim 52, wherein the method further comprises administering a second therapeutic agent.

57. The method of claim 56, wherein the second therapeutic agent is a chemotherapeutic agent, tyrosine kinase inhibitor, an antibody, or a cytokine.

58. (canceled)

59. A method for diagnosing a subject with a KIT-mediated disorder comprising contacting cells or a sample obtained from the subject with the isolated antibody of claim 1 and detecting the expression level of KIT in the cells or the sample.

60. (canceled)

61. (canceled)

62. A method for inhibiting KIT activity in a cell expressing KIT comprising contacting the cell with an effective amount of the isolated antibody of claim 1.

63. (canceled)

64. (canceled)

65. (canceled)

66. (canceled)

67. (canceled)

68. (canceled)

69. (canceled)

70. (canceled)

71. (canceled)

72. (canceled)

73. (canceled)
